SEQUENCE LISTING (237 sequences)

SEQ ID NO: 1 - DNA, 9660 bp, Homo sapiens
  source 1..9660: mol_type = unassigned DNA
ataaattcattgttccacctcctcgcatcttcacagcgctcgcgctgctctcggcgctcgcagctgccgactggggatgacggcgggcaggaggagaccgcagccgaagggacacagacacgccgcttcaccagctcgcctcaggctgcccccctgcatttttgttttaatttttacggctttttcccctctctttcttcccttcctcctggtcccagcagagccaaggaaacccacaaaataagaaaggaagtgggccccggagcttggaacctccacagccggcttgtccagcgcagcgcgggggcgggaggctgcgcgcaccagttgccagcccggtgcgcggtacctttccttacttttcttgaaacagcgatcgtgcctgcatttggtggttttttggtttttgtttttttccttttcccgtatttgctgaatctccactatccgactttttttttttaatcttttctttccccccccccccaccccacctctttctggagcacgaatccaaacattttcccaagcaacaaagaaaagttcgcacgctggcaccgcagcccggacaggctggcgctgctgccgggcccccctccctccgacacttgactcaatcctgcaagcaagtgtgtgtgtgtccccatcccccgccccgttaacttcatagcaaataacaaatacccataaagtcccagtcgcgcagcccctccccgcgggcagcgcactatgctgctcgggtgggcgtccctgctgctgtgcgcgttccgcctgcccctggccgcggtcggccccgccgcgacacctgcccaggataaagccgggcagcctccgactgctgcagcagccgcccagccccgccggcggcagggggaggaggtgcaggagcgagccgagcctcccggccacccgcaccccctggcgcagcggcgcaggagcaaggggctggtgcagaacatcgaccaactctactccggcggcggcaaggtgggctacctcgtctacgcgggcggccggaggttcctcttggacctggagcgagatggttcggtgggcattgctggcttcgtgcccgcaggaggcgggacgagtgcgccctggcgccaccggagccactgcttctatcggggcacagtggacggtagtccccgctctctggctgtctttgacctctgtgggggtctcgacggcttcttcgcggtcaagcacgcgcgctacaccctaaagccactgctgcgcggaccctgggcggaggaagaaaaggggcgcgtgtacggggatgggtccgcacggatcctgcacgtctacacccgcgagggcttcagcttcgaggccctgccgccgcgcgccagctgcgaaacccccgcgtccacaccggaggcccacgagcatgctccggcgcacagcaacccgagcggacgcgcagcactggcctcgcagctcttggaccagtccgctctctcgcccgctgggggctcaggaccgcagacgtggtggcggcggcggcgccgctccatctcccgggcccgccaggtggagctgcttctggtggctgacgcgtccatggcgcggttgtatggccggggcctgcagcattacctgctgaccctggcctccatcgccaataggctgtacagccatgctagcatcgagaaccacatccgcctggccgtggtgaaggtggtggtgctaggcgacaaggacaagagcctggaagtgagcaagaacgctgccaccacactcaagaacttttgcaagtggcagcaccaacacaaccagctgggagatgaccatgaggagcactacgatgcagctatcctgtttactcgggaggatttatgtgggcatcattcatgtgacaccctgggaatggcagacgttgggaccatatgttctccagagcgcagctgtgctgtgattgaagacgatggcctccacgcagccttcactgtggctcacgaaatcggacatttacttggcctctcccatgacgattccaaattctgtgaagagacctttggttccacagaagataagcgcttaatgtcttccatccttaccagcattgatgcatctaagccctggtccaaatgcacttcagccaccatcacagaattcctggatgatggccatggtaactgtttgctggacctaccacgaaagcagatcctgggccccgaagaactcccaggacagacctacgatgccacccagcagtgcaacctgacattcgggcctgagtactccgtgtgtcccggcatggatgtctgtgctcgcctgtggtgtgctgtggtacgccagggccagatggtctgtctgaccaagaagctgcctgcggtggaagggacgccttgtggaaaggggagaatctgcctgcagggcaaatgtgtggacaaaaccaagaaaaaatattattcaacgtcaagccatggcaactggggatcttggggatcctggggccagtgttctcgctcatgtggaggaggagtgcagtttgcctatcgtcactgtaataaccctgctcccagaaacaacggacgctactgcacagggaagagggccatctaccgctcctgcagtctcatgccctgcccacccaatggtaaatcatttcgtcatgaacagtgtgaggccaaaaatggctatcagtctgatgcaaaaggagtcaaaacttttgtggaatgggttcccaaatatgcaggtgtcctgccagcggatgtgtgcaagctgacctgcagagccaagggcactggctactatgtggtattttctccaaaggtgaccgatggcactgaatgtaggctgtacagtaattccgtctgcgtccgggggaagtgtgtgagaactggctgtgacggcatcattggctcaaagctgcagtatgacaagtgcggagtatgtggaggagacaactccagctgtacaaagattgttggaacctttaataagaaaagtaagggttacactgacgtggtgaggattcctgaaggggcaacccacataaaagttcgacagttcaaagccaaagaccagactagattcactgcctatttagccctgaaaaagaaaaacggtgagtaccttatcaatggaaagtacatgatctccacttcagagactatcattgacatcaatggaacagtcatgaactatagcggttggagccacagggatgacttcctgcatggcatgggctactctgccacgaaggaaattctaatagtgcagattcttgcaacagaccccactaaaccattagatgtccgttatagcttttttgttcccaagaagtccactccaaaagtaaactctgtcactagtcatggcagcaataaagtgggatcacacacttcgcagccgcagtgggtcacgggcccatggctcgcctgctctaggacctgtgacacaggttggcacaccagaacggtgcagtgccaggatggaaaccggaagttagcaaaaggatgtcctctctcccaaaggccttctgcgtttaagcaatgcttgttgaagaaatgttagcctgtggttatgatcttatgcacaaagataactggaggattcagcactgatgcagtcgtggtgaacaggaggtctacctaacgcacagaaagtcatgcttcagtgacattgtcaacaggagtccaattatgggcagaatctgctctctgtgaccaaaagaggatgtgcactgcttcacgtgacagtggtgaccttgcaatatagaaaaacttgggagttattgaacatcccctgggcttacaagaaacactgatgaatgtaaaatcaggggacatttgaagatggcagaactgtctcccccttgtcacctacctctgatagaatgtctttaatggtatcataatcattttcacccataatacacagtagcttcttcttactgtttgtaaatacattctcccttggtatgtcactttatatcccctggttctattaaaatatccatatatatttctataaaaaaagtgtttgaccaaagtaggtctgcagctatttcaacttccttccgtttccagaaagagctgtggatattttactggaaattaagaacttgctgctgttttaataagatgtagtatattttctgactacaggagataaaatttcagtcaaaaaaccattttgacagcaagtatcttctgagaaattttgaaaagtaaatagatctcagtgtatctagtcacttaaatacatacacgggttcatttacttaaacctttgactgcctgtatttttttcaggtagctagccaaattaatgcataatttcagatgtagaagtagggtttgcgtgtgtgtgtgtgatcatactcaagagtctaaaaactagtttccttgtgttggaaatttaaaaggaaaaaaatcgtatttcactgtgttttcaatttatattttcacaactactttctctctccagagctttcatctgatatctcacaatgtatgatatacgtacaaaacacacagcaagttttctatcatgtccaacacattcaacactggtatacctcctaccagcaagcctttaaaatgcatttgtgtttgcttatttgttttgttcaagggttcagtaagacctacaatgttttgtatttcttgacttattttattagaaacattaaagatcacttggtagttagccacattgagaagtggttatcattgttaatgtggttaatgccaaaaagtggttaatattaataagactgtttccacaccataggcaataatttcttaatttaaaaaatctaagtatattcctattgtactaaatatttttcccaactggaaagcacttgattgtacccgtaagtgtttgagtgatgacatgtgatgattttcagaaagttgttgtttttgtttccatagcctgtttaagtaggttgtaagtttgaatagttagacatggaaattattttataagcacacacctaaagatatctttttagatgataaaatgtacacccccccatcaccaacctcacaacttagaaaatctaagttgtttgatttctttgggatttcttttgttgtgaaacactgcaaagccaatttttctttataaaaattcatagtaatcctgccaaatgtgcctattgttaaagatttgcatgtgaagatcttagggaaccactgtttgagttctacaagctcatgagagtttatttttattataagatgtttttaatataaaagaattatgtaactgatcactatattacatcatttcagtgggccaggaaaatagatgtcttgctgttttcagtattttcttaagaaattgcttttaaaacaaataattgttttacaaaaccaataattatcctttgaattttcatagactgactttgcttttgacgtagaaattttttttctcaataaattatcactttgagaaatgaggcctgtacaaggctgataacctatatgtgatggagatcacccaatgccaagggcagaaagcaaacctagttaaataggtgagaaaaaaaataataatcccagtgccatttgtctgtgcaaagagaattaggagagaggttaatgttacttttttccattttggaaataattttaatcaagtaactcaaatgtgacaaaatttatttttattttttgtggttatattcccaacaacattaaaaaatactcgaggcataaatgtagttgtctcctactctgcttctcttactatactcatacatttttaatatggtttatcaatgattcatgtttccctcaaatagtgatggtttacacctgtcatggaaacaatcctagagagctcagagcaattaaaccactattccatgcttttaagtagttttctccacctttttcttatgagtctcactagattgactgaggaatgtatgtctaaattcctggagaagatgatatggattggaaactgaaattcagagaaatggagtgttcaatagataccacgaattgtgaacaaagggaaaattctatacaactcaatctaagtcagtccactttgacttcgtactgtctttcacctttccattgttgcatcttgaattttttaaaatgtctagaattcaggatgctaggggctacttctttaaaaaaaaaaaaaaaaaagaattcgtctgaaaatgctcaggtttgtaagaatctaatctcacttacataactaagcactccataataagttttattaagtacaaagggagccagaaaaaatgacatttatttcttctagatcagaaaaatttaaattaagccctgccttgctgtttagaaatatgtgggcattgttataatttattcaataaatttatgttcctttgccttcctgtggaaacagttttatcccactaaactaggaattaggggataaatcacaaacaaaaaaaaagttgcagcactgaaaaaaagtaatttattgtttttgcaactggtatgtgaatttgtgtgataaaattatttattcttatttaacaaaaatatgttcaaatttttctatatttaaaatgttttgctgttgtcctactttttaatttatgcttcatgtttgtgtataaagtacacttttacactttgtgagtttacataatatacagcactggttgcttttgtatttttttacagaaagctttctgtgtgaagcaggtgtatatgtatatattcctcatgtattcttattctgatactatcatttttctttccaaggaaattttaatctgtcatgaccaatagtgttcattacttgtgcctatgataataggttttttacatcacattaacactattttttccaagtcacaaataagaaaaacacttattcaatgaaacaaggtgcaagttttaaatttgggtacacaaatagcctagaagcttcctacagacgctaagacacagccaataatcagatcctttcacttcatcgagaaacttggacaagtcgatattgatgtattagatgaaagttgtctacacacaacttctgagggatacaaacgataataaaaccaaatgttgtctgtttctcctttagaaacacctcctaaaattaatatcatttagtctctagtgtctgtaggattctacagatgagcacaaatagattgggtttgtataacaaatgctaatagtcataactgtttctacaaatatggggtgtccattaagagaatgtgatgttttcctactgctgttgaatcccatggggtgattataggacttgaaataggcagagtcacctctgatgacatcagcttgcctctgtgatttcacagtctgatcctggcaacaagacaaagcacccttggacacacagccaatctctggttgtgatatttccccattgattccttccttgttaacaaggtcattttaatggttcaggtgaggacagcagccagattcaaagtccagaatttgtgctgttacatagagttcacactgtcaaataacattgaatttaataatgatcaaatttttctagtagtctttggcagagtgtataatctcattggcatgattggtgaatattactaatctctttataatgaaagatgctttacaaataccttatatttgctaacatttcaaaactactaaataaatgaaatagccatgtgtacagaaatggtcatttaaagctttaatagaaccaaattcaagacaatgtatcatttagacacacagaaaaggaacttgtatgttttccctattatttttctcatttgccaacaatctatagttttaggttatcaaacagatagatcaacttaactggctagtacattgaaaaatcttcctaagaatcctttgttagcataatctatagagataatttctcaaattatatcatcatgatgcatataaactctataatgtataattgtgtttcatttatttaatgtatgagaacatattgaaatacaaaaccatgcattagccaaaaaattggaatacaggtagtgttcagatcagcaaaacattcagtctggtaaatgcctgcctggggctatgatatcattctcaatgcaggttttatggaaaaactaaaagaatatgttgttagatgatgttggttttgaaaaaaaaaagacattaacatacacattagttagcccagttaattgcattctactaatatagttgcacattagcaataattttgctgtctctggtctttattttgtggcttcaactaactggaccatgtggactgtaaaggtcaaatggaaaaaacgagcagtggcccctcatcctgtaaggtactgctacatcagagtgacctaaaagtctaacactgtgaggaaaactgtgatttgtaggaaaaaaaaaaaaaacaaataaaaaacagggcatgctttttaatttttttccactttcctttggcacacccaatgaacaattctaatttttattgaggtgctaacatctttcgtgaccgactgtcaaatgtggtatttttgagttactatttttctacatgattttacagtttgcaagaaagacctctaagctttgtgtcacggtagggcacaacttgatactcaaaatttgaaaaataagcacatccaatgattgttttgaccaacagtggtcagtgacgtaaactgcatgtgcatctgaggacatttaaggggtcattaaaatttgaggagcatcaggccggagtagcagacttttagatgagtcatatttcagcattcactaagtcctcagcattccattcaaactgtcgtgtatatttggcctgattttttttcaagctttgcaataatttatgttattggtaaacacttggtgactatatctcagccttttctttaacaactcacaatatattagaaacacgtctacctatactgagagtatatttacaatagaagaacatactgtatgtgactttgtaaagctagacttttgattaagaaatatataatctctggatgctatttttgcattatacactcaggcacaacgtaaaccttgatggctcatcttgctacaattacgagttgaaaaacactacttacgtatttgtatgacctattagtcagaggaaatcatacatatgctttgtaaatagactttgcagataactaaatagactgaagaaatatgttgcatttgatagaagcaattgcataaatatttggtttctatattagagtctgtgagtaaagtcaagtaataaacctaagtaggtataacagatttttaaaccttgaaacttgctttgatggtagagaaaatcattgaagatttacatactgtatataagatgtaaaatgtacgctgcttattaccctcaattttccagaagcaatggtatataatgcagttgaaaaaccaaaaatcttggaaaactaagacgggtcttgtttaaaatgtctctcagctttggcaaccttcaaatcttaatcaactatttaaagcattactgtgtcttgtagcctgcattccacaacagctctgttattcaggtaaaagacttgaactgagccgtttgggacctatactgtaatattttcattgaggaacaatatcctattttgtaaagcatttccctatgtgtgactttaaactgtaaaattaaacactgcttttgtgggttcagtgggcataataaatataaattgtaaactaggttaaa

SEQ ID NO: 2 - protein, 930 aa, Homo sapiens
MLLGWASLLLCAFRLPLAAVGPAATPAQDKAGQPPTAAAAAQPRRRQGEEVQERAEPPGHPHPLAQRRRSKGLVQNIDQLYSGGGKVGYLVYAGGRRFLLDLERDGSVGIAGFVPAGGGTSAPWRHRSHCFYRGTVDGSPRSLAVFDLCGGLDGFFAVKHARYTLKPLLRGPWAEEEKGRVYGDGSARILHVYTREGFSFEALPPRASCETPASTPEAHEHAPAHSNPSGRAALASQLLDQSALSPAGGSGPQTWWRRRRRSISRARQVELLLVADASMARLYGRGLQHYLLTLASIANRLYSHASIENHIRLAVVKVVVLGDKDKSLEVSKNAATTLKNFCKWQHQHNQLGDDHEEHYDAAILFTREDLCGHHSCDTLGMADVGTICSPERSCAVIEDDGLHAAFTVAHEIGHLLGLSHDDSKFCEETFGSTEDKRLMSSILTSIDASKPWSKCTSATITEFLDDGHGNCLLDLPRKQILGPEELPGQTYDATQQCNLTFGPEYSVCPGMDVCARLWCAVVRQGQMVCLTKKLPAVEGTPCGKGRICLQGKCVDKTKKKYYSTSSHGNWGSWGSWGQCSRSCGGGVQFAYRHCNNPAPRNNGRYCTGKRAIYRSCSLMPCPPNGKSFRHEQCEAKNGYQSDAKGVKTFVEWVPKYAGVLPADVCKLTCRAKGTGYYVVFSPKVTDGTECRLYSNSVCVRGKCVRTGCDGIIGSKLQYDKCGVCGGDNSSCTKIVGTFNKKSKGYTDVVRIPEGATHIKVRQFKAKDQTRFTAYLALKKKNGEYLINGKYMISTSETIIDINGTVMNYSGWSHRDDFLHGMGYSATKEILIVQILATDPTKPLDVRYSFFVPKKSTPKVNSVTSHGSNKVGSHTSQPQWVTGPWLACSRTCDTGWHTRTVQCQDGNRKLAKGCPLSQRPSAFKQCLLKKC

SEQ ID NO: 3 - RNA, 19 nt, synthetic construct
  misc_feature 1..19: note = synthetic construct
  source 1..19: mol_type = other RNA
gctcaaagctgcagtatga

SEQ ID NO: 4 - RNA, 19 nt, synthetic construct
  misc_feature 1..19: note = synthetic construct
  source 1..19: mol_type = other RNA
gaagtccactccaaaagta

SEQ ID NO: 5 - RNA, 19 nt, synthetic construct
  misc_feature 1..19: note = synthetic construct
  source 1..19: mol_type = other RNA
gcactacgatgcagctatc

SEQ ID NO: 6 - RNA, 19 nt, synthetic construct
  misc_feature 1..19: note = synthetic construct
  source 1..19: mol_type = other RNA
cgaaggaaattctaatagt

SEQ ID NO: 7 - DNA, 32 bp, synthetic construct
  misc_feature 1..32: note = synthetic construct
  source 1..32: mol_type = other DNA
ccggtctaacatttcttcaacaagcagaccgg

SEQ ID NO: 8 - DNA, 32 bp, synthetic construct
  misc_feature 1..32: note = synthetic construct
  source 1..32: mol_type = other DNA
ccggtcttatacacaaacatgaagcagaccgg

SEQ ID NO: 9 - DNA, 32 bp, synthetic construct
  misc_feature 1..32: note = synthetic construct
  source 1..32: mol_type = other DNA
ccggtctacatcttattaaaacagcagaccgg

SEQ ID NO: 10 - DNA, 4410 bp, Homo sapiens
  source 1..4410: mol_type = unassigned DNA
ggggagaacccacagggagacccacagacacatatgcacgagagagacagaggaggaaagagacagagacaaaggcacagcggaagaaggcagagacagggcaggcacagaagcggcccagacagagtcctacagagggagaggccagagaagctgcagaagacacaggcagggagagacaaagatccaggaaaggagggctcaggaggagagtttggagaagccagacccctgggcacctctcccaagcccaaggactaagttttctccatttcctttaacggtcctcagcccttctgaaaactttgcctctgaccttggcaggagtccaagcccccaggctacagagaggagctttccaaagctagggtgtggaggacttggtgccctagacggcctcagtccctcccagctgcagtaccagtgccatgtcccagacaggctcgcatcccgggaggggcttggcagggcgctggctgtggggagcccaaccctgcctcctgctccccattgtgccgctctcctggctggtgtggctgcttctgctactgctggcctctctcctgccctcagcccggctggccagccccctcccccgggaggaggagatcgtgtttccagagaagctcaacggcagcgtcctgcctggctcgggcgcccctgccaggctgttgtgccgcttgcaggcctttggggagacgctgctactagagctggagcaggactccggtgtgcaggtcgaggggctgacagtgcagtacctgggccaggcgcctgagctgctgggtggagcagagcctggcacctacctgactggcaccatcaatggagatccggagtcggtggcatctctgcactgggatgggggagccctgttaggcgtgttacaatatcggggggctgaactccacctccagcccctggagggaggcacccctaactctgctgggggacctggggctcacatcctacgccggaagagtcctgccagcggtcaaggtcccatgtgcaacgtcaaggctcctcttggaagccccagccccagaccccgaagagccaagcgctttgcttcactgagtagatttgtggagacactggtggtggcagatgacaagatggccgcattccacggtgcggggctaaagcgctacctgctaacagtgatggcagcagcagccaaggccttcaagcacccaagcatccgcaatcctgtcagcttggtggtgactcggctagtgatcctggggtcaggcgaggaggggccccaagtggggcccagtgctgcccagaccctgcgcagcttctgtgcctggcagcggggcctcaacacccctgaggactcggaccctgaccactttgacacagccattctgtttacccgtcaggacctgtgtggagtctccacttgcgacacgctgggtatggctgatgtgggcaccgtctgtgacccggctcggagctgtgccattgtggaggatgatgggctccagtcagccttcactgctgctcatgaactgggtcatgtcttcaacatgctccatgacaactccaagccatgcatcagtttgaatgggcctttgagcacctctcgccatgtcatggcccctgtgatggctcatgtggatcctgaggagccctggtccccctgcagtgcccgcttcatcactgacttcctggacaatggctatgggcactgtctcttagacaaaccagaggctccattgcatctgcctgtgactttccctggcaaggactatgatgctgaccgccagtgccagctgaccttcgggcccgactcacgccattgtccacagctgccgccgccctgtgctgccctctggtgctctggccacctcaatggccatgccatgtgccagaccaaacactcgccctgggccgatggcacaccctgcgggcccgcacaggcctgcatgggtggtcgctgcctccacatggaccagctccaggacttcaatattccacaggctggtggctggggtccttggggaccatggggtgactgctctcggacctgtgggggtggtgtccagttctcctcccgagactgcacgaggcctgtcccccggaatggtggcaagtactgtgagggccgccgtacccgcttccgctcctgcaacactgaggactgcccaactggctcagccctgaccttccgcgaggagcagtgtgctgcctacaaccaccgcaccgacctcttcaagagcttcccagggcccatggactgggttcctcgctacacaggcgtggccccccaggaccagtgcaaactcacctgccaggcccaggcactgggctactactatgtgctggagccacgggtggtagatgggaccccctgttccccggacagctcctcggtctgtgtccagggccgatgcatccatgctggctgtgatcgcatcattggctccaagaagaagtttgacaagtgcatggtgtgcggaggggacggttctggttgcagcaagcagtcaggctccttcaggaaattcaggtacggatacaacaatgtggtcactatccccgcgggggccacccacattcttgtccggcagcagggaaaccctggccaccggagcatctacttggccctgaagctgccagatggctcctatgccctcaatggtgaatacacgctgatgccctcccccacagatgtggtactgcctggggcagtcagcttgcgctacagcggggccactgcagcctcagagacactgtcaggccatgggccactggcccagcctttgacactgcaagtcctagtggctggcaacccccaggacacacgcctccgatacagcttcttcgtgccccggccgaccccttcaacgccacgccccactccccaggactggctgcaccgaagagcacagattctggagatccttcggcggcgcccctgggcgggcaggaaataacctcactatcccggctgccctttctgggcaccggggcctcggacttagctgggagaaagagagagcttctgttgctgcctcatgctaagactcagtggggaggggctgtgggcgtgagacctgcccctcctctctgccctaatgcgcaggctggccctgccctggtttcctgccctgggaggcagtgatgggttagtggatggaaggggctgacagacagccctccatctaaactgccccctctgccctgcgggtcacaggagggagggggaaggcagggagggcctgggccccagttgtatttatttagtatttattcacttttatttagcaccagggaaggggacaaggactagggtcctggggaacctgacccctgacccctcatagccctcaccctggggctaggaaatccagggtggtggtgataggtataagtggtgtgtgtatgcgtgtgtgtgtgtgtgaaaatgtgtgtgtgcttatgtatgaggtacaacctgttctgctttcctcttcctgaattttattttttgggaaaagaaaagtcaagggtagggtgggccttcagggagtgagggattatctttttttttttttctttctttctttcttttttttttttgagacagaatctcgctctgtcgcccaggctggagtgcaatggcacaatctcggctcactgcatcctccgcctcccgggttcaagtgattctcatgcctcagcctcctgagtagctgggattacaggctcctgccaccacgcccggctaatttttgttttgttttgtttggagacagagtctcgctattgtcaccagggctggaatgatttcagctcactgcaaccttcgccacctgggttccagcaattctcctgcctcagcctcccgagtagctgagattataggcacctaccaccacgcccggctaatttttgtatttttagtagagacggggtttcaccatgttggccaggctggtctcgaactcctgaccttaggtgatccactcgccttcatctcccaaagtgctgggattacaggcgtgagccaccgtgcctggccacgcccaactaatttttgtatttttagtagagacagggtttcaccatgttggccaggctgctcttgaactcctgacctcaggtaatcgacctgcctcggcctcccaaagtgctgggattacaggtgtgagccaccacgcccggtacatattttttaaattgaattctactatttatgtgatccttttggagtcagacagatgtggttgcatcctaactccatgtctctgagcattagatttctcatttgccaataataatacctcccttagaagtttgttgtgaggattaaataatgtaaataaagaactagcataacactcaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 11 - protein, 837 aa, Homo sapiens
MSQTGSHPGRGLAGRWLWGAQPCLLLPIVPLSWLVWLLLLLLASLLPSARLASPLPREEEIVFPEKLNGSVLPGSGAPARLLCRLQAFGETLLLELEQDSGVQVEGLTVQYLGQAPELLGGAEPGTYLTGTINGDPESVASLHWDGGALLGVLQYRGAELHLQPLEGGTPNSAGGPGAHILRRKSPASGQGPMCNVKAPLGSPSPRPRRAKRFASLSRFVETLVVADDKMAAFHGAGLKRYLLTVMAAAAKAFKHPSIRNPVSLVVTRLVILGSGEEGPQVGPSAAQTLRSFCAWQRGLNTPEDSDPDHFDTAILFTRQDLCGVSTCDTLGMADVGTVCDPARSCAIVEDDGLQSAFTAAHELGHVFNMLHDNSKPCISLNGPLSTSRHVMAPVMAHVDPEEPWSPCSARFITDFLDNGYGHCLLDKPEAPLHLPVTFPGKDYDADRQCQLTFGPDSRHCPQLPPPCAALWCSGHLNGHAMCQTKHSPWADGTPCGPAQACMGGRCLHMDQLQDFNIPQAGGWGPWGPWGDCSRTCGGGVQFSSRDCTRPVPRNGGKYCEGRRTRFRSCNTEDCPTGSALTFREEQCAAYNHRTDLFKSFPGPMDWVPRYTGVAPQDQCKLTCQAQALGYYYVLEPRVVDGTPCSPDSSSVCVQGRCIHAGCDRIIGSKKKFDKCMVCGGDGSGCSKQSGSFRKFRYGYNNVVTIPAGATHILVRQQGNPGHRSIYLALKLPDGSYALNGEYTLMPSPTDVVLPGAVSLRYSGATAASETLSGHGPLAQPLTLQVLVAGNPQDTRLRYSFFVPRPTPSTPRPTPQDWLHRRAQILEILRRRPWAGRK

SEQ ID NO: 12 - RNA, 19 nt, synthetic construct
  misc_feature 1..19: note = synthetic construct
  source 1..19: mol_type = other RNA
ccgcaatcctgtcagcttg

SEQ ID NO: 13 - RNA, 19 nt, synthetic construct
  misc_feature 1..19: note = synthetic construct
  source 1..19: mol_type = other RNA
gcgctttgcttcactgagt

SEQ ID NO: 14 - RNA, 19 nt, synthetic construct
  misc_feature 1..19: note = synthetic construct
  source 1..19: mol_type = other RNA
ggacacacgcctccgatac

SEQ ID NO: 15 - RNA, 19 nt, synthetic construct
  misc_feature 1..19: note = synthetic construct
  source 1..19: mol_type = other RNA
gcaccgaagagcacagatt

SEQ ID NO: 16 - DNA, 32 bp, synthetic construct
  misc_feature 1..32: note = synthetic construct
  source 1..32: mol_type = other DNA
ccggtcttttcacacacacacacacggaccgg

SEQ ID NO: 17 - DNA, 32 bp, synthetic construct
  misc_feature 1..32: note = synthetic construct
  source 1..32: mol_type = other DNA
ccggtctaaaaatacaaaaattagccgaccgg

SEQ ID NO: 18 - DNA, 32 bp, synthetic construct
  misc_feature 1..32: note = synthetic construct
  source 1..32: mol_type = other DNA
ccggtcttgtctctgtctctttcctcgaccgg

SEQ ID NO: 19 - DNA, 2735 bp, Homo sapiens
  source 1..2735: mol_type = unassigned DNA
acaacagtccccaggcatcaccattcaagatgcatccaggggtcctggctgccttcctcttcttgagctggactcattgtcgggccctgccccttcccagtggtggtgatgaagatgatttgtctgaggaagacctccagtttgcagagcgctacctgagatcatactaccatcctacaaatctcgcgggaatcctgaaggagaatgcagcaagctccatgactgagaggctccgagaaatgcagtctttcttcggcttagaggtgactggcaaacttgacgataacaccttagatgtcatgaaaaagccaagatgcggggttcctgatgtgggtgaatacaatgttttccctcgaactcttaaatggtccaaaatgaatttaacctacagaattgtgaattacacccctgatatgactcattctgaagtcgaaaaggcattcaaaaaagccttcaaagtttggtccgatgtaactcctctgaattttaccagacttcacgatggcattgctgacatcatgatctcttttggaattaaggagcatggcgacttctacccatttgatgggccctctggcctgctggctcatgcttttcctcctgggccaaattatggaggagatgcccattttgatgatgatgaaacctggacaagtagttccaaaggctacaacttgtttcttgttgctgcgcatgagttcggccactccttaggtcttgaccactccaaggaccctggagcactcatgtttcctatctacacctacaccggcaaaagccactttatgcttcctgatgacgatgtacaagggatccagtctctctatggtccaggagatgaagaccccaaccctaaacatccaaaaacgccagacaaatgtgacccttccttatcccttgatgccattaccagtctccgaggagaaacaatgatctttaaagacagattcttctggcgcctgcatcctcagcaggttgatgcggagctgtttttaacgaaatcattttggccagaacttcccaaccgtattgatgctgcatatgagcacccttctcatgacctcatcttcatcttcagaggtagaaaattttgggctcttaatggttatgacattctggaaggttatcccaaaaaaatatctgaactgggtcttccaaaagaagttaagaagataagtgcagctgttcactttgaggatacaggcaagactctcctgttctcaggaaaccaggtctggagatatgatgatactaaccatattatggataaagactatccgagactaatagaagaagacttcccaggaattggtgataaagtagatgctgtctatgagaaaaatggttatatctattttttcaacggacccatacagtttgaatacagcatctggagtaaccgtattgttcgcgtcatgccagcaaattccattttgtggtgttaagtgtctttttaaaaattgttatttaaatcctgaagagcatttggggtaatacttccagaagtgcggggtaggggaagaagagctatcaggagaaagcttggttctgtgaacaagcttcagtaagttatctttgaatatgtagtatctatatgactatgcgtggctggaaccacattgaagaatgttagagtaatgaaatggaggatctctaaagagcatctgattcttgttgctgtacaaaagcaatggttgatgatacttcccacaccacaaatgggacacatggtctgtcaatgagagcataatttaaaaatatatttataaggaaattttacaagggcataaagtaaatacatgcatataatgaataaatcattcttactaaaaagtataaaatagtatgaaaatggaaatttgggagagccatacataaaagaaataaaccaaaggaaaatgtctgtaataatagactgtaacttccaaataaataattttcattttgcactgaggatattcagatgtatgtgcccttcttcacacagacactaacgaaatatcaaagtcattaaagacaggagacaaaagagcagtggtaagaatagtagatgtggcctttgaattctgtttaattttcacttttggcaatgactcaaagtctgctctcatataagacaaatattcctttgcatattataaaggataaagaaggatgatgtctttttattaaaatatttcaggttcttcagaagtcacacattacaaagttaaaattgttatcaaaatagtctaaggccatggcatccctttttcataaattatttgattatttaagactaaaagttgcattttaaccctattttacctagctaattatttaattgtccagtttgtcttggatatataggctattttctaaagacttgtatagcatgaaataaaatatatcttataaagtggaagtatgtatattaaaaaagagacatccaaatttttttttaaagcagtctactagattgtgatcccttgagatatggaaggatgcctttttttctctgcatttaaaaaaatcccccagcacttcccacagtgcctattgatacttggggagggtgcttggcacttattgaatatatgatcggccatcaagggaagaactattgtgctcagagacactgttgataaaaactcaggcaaagaaaatgaaatgcatatttgcaaagtgtattaggaagtgtttatgttgtttataataaaaatatattttcaacagacaaaaaaaaaaaaaaaaaa

SEQ ID NO: 20 - protein, 471 aa, Homo sapiens
MHPGVLAAFLFLSWTHCRALPLPSGGDEDDLSEEDLQFAERYLRSYYHPTNLAGILKENAASSMTERLREMQSFFGLEVTGKLDDNTLDVMKKPRCGVPDVGEYNVFPRTLKWSKMNLTYRIVNYTPDMTHSEVEKAFKKAFKVWSDVTPLNFTRLHDGIADIMISFGIKEHGDFYPFDGPSGLLAHAFPPGPNYGGDAHFDDDETWTSSSKGYNLFLVAAHEFGHSLGLDHSKDPGALMFPIYTYTGKSHFMLPDDDVQGIQSLYGPGDEDPNPKHPKTPDKCDPSLSLDAITSLRGETMIFKDRFFWRLHPQQVDAELFLTKSFWPELPNRIDAAYEHPSHDLIFIFRGRKFWALNGYDILEGYPKKISELGLPKEVKKISAAVHFEDTGKTLLFSGNQVWRYDDTNHIMDKDYPRLIEEDFPGIGDKVDAVYEKNGYIYFFNGPIQFEYSIWSNRIVRVMPANSILWC

SEQ ID NO: 21 - RNA, 20 nt, synthetic construct
  misc_feature 1..20: note = synthetic construct
  source 1..20: mol_type = other RNA
tttcacacacacacacacgc

SEQ ID NO: 22 - RNA, 20 nt, synthetic construct
  misc_feature 1..20: note = synthetic construct
  source 1..20: mol_type = other RNA
ttttcacacacacacacacg

SEQ ID NO: 23 - RNA, 20 nt, synthetic construct
  misc_feature 1..20: note = synthetic construct
  source 1..20: mol_type = other RNA
taaaaatacaaaaattagcc

SEQ ID NO: 24 - RNA, 20 nt, synthetic construct
  misc_feature 1..20: note = synthetic construct
  source 1..20: mol_type = other RNA
tttgtctctgtctctttcct

SEQ ID NO: 25 - DNA, 32 bp, synthetic construct
  misc_feature 1..32: note = synthetic construct
  source 1..32: mol_type = other DNA
ccggtctacacacaccacttatacctgaccgg

SEQ ID NO: 26 - DNA, 32 bp, synthetic construct
  misc_feature 1..32: note = synthetic construct
  source 1..32: mol_type = other DNA
ccggtctataatctcagctactcggggaccgg

SEQ ID NO: 27 - DNA, 32 bp, synthetic construct
  misc_feature 1..32: note = synthetic construct
  source 1..32: mol_type = other DNA
ccggtcaaacaaaacaaaaattagccgaccgg

SEQ ID NO: 28 - DNA, 1903 bp, Homo sapiens
  source 1..1903: mol_type = unassigned DNA
agcatgagtcagacagcctctggctttctggaagggcaaggactctatatatacagagggagcttcctagctgggatattggagcagcaagaggctgggaagccatcacttaccttgcactgagaaagaagacaaaggcaagttgaaaagcggagaaatagtggcccagtggttgaaaaattgaagcaaatgcaggaattctttgggctgaaagtgactgggaaaccagatgctgaaaccctgaaggtgatgaagcagcccagatgtggagtgcctgatgtggctcagtttgtcctcactgaggggaaccctcgctgggagcaaacacatctgacctacaggattgaaaattacacgccagatttgccaagagcagatgtggaccatgccattgagaaagccttccaactctggagtaatgtcacacctctgacattcaccaaggtctctgagggtcaagcagacatcatgatatcttttgtcaggggagatcatcgggacaactctccttttgatggacctggaggaaatcttgctcatgcttttcaaccaggcccaggtattggaggggatgctcattttgatgaagatgaaaggtggaccaacaatttcagagagtacaacttacatcgtgttgcagctcatgaactcggccattctcttggactctcccattctactgatatcggggctttgatgtaccctagctacaccttcagtggtgatgttcagctagctcaggatgacattgatggcatccaagccatatatggacgttcccaaaatcctgtccagcccatcggcccacaaaccccaaaagcgtgtgacagtaagctaacctttgatgctataactacgattcggggagaagtgatgttctttaaagacagattctacatgcgcacaaatcccttctacccggaagttgagctcaatttcatttctgttttctggccacaactgccaaatgggcttgaagctgcttacgaatttgccgacagagatgaagtccggtttttcaaagggaataagtactgggctgttcagggacagaatgtgctacacggataccccaaggacatctacagctcctttggcttccctagaactgtgaagcatatcgatgctgctctttctgaggaaaacactggaaaaacctacttctttgttgctaacaaatactggaggtatgatgaatataaacgatctatggatccaggttatcccaaaatgatagcacatgactttcctggaattggccacaaagttgatgcagttttcatgaaagatggatttttctatttctttcatggaacaagacaatacaaatttgatcctaaaacgaagagaattttgactctccagaaagctaatagctggttcaactgcaggaaaaattgaacattactaatttgaatggaaaacacatggtgtgagtccaaagaaggtgttttcctgaagaactgtctattttctcagtcatttttaacctctagagtcactgatacacagaatataatcttatttatacctcagtttgcatatttttttactatttagaatgtagccctttttgtactgatataatttagttccacaaatggtgggtacaaaaagtcaagtttgtggcttatggattcatataggccagagttgcaaagatcttttccagagtatgcaactctgacgttgatcccagagagcagcttcagtgacaaacatatcctttcaagacagaaagagacaggagacatgagtctttgccggaggaaaagcagctcaagaacacatgtgcagtcactggtgtcaccctggataggcaagggataactcttctaacacaaaataagtgttttatgtttggaataaagtcaaccttgtttctactgttttatacactttc

SEQ ID NO: 29 - protein, 403 aa, Homo sapiens
MQEFFGLKVTGKPDAETLKVMKQPRCGVPDVAQFVLTEGNPRWEQTHLTYRIENYTPDLPRADVDHAIEKAFQLWSNVTPLTFTKVSEGQADIMISFVRGDHRDNSPFDGPGGNLAHAFQPGPGIGGDAHFDEDERWTNNFREYNLHRVAAHELGHSLGLSHSTDIGALMYPSYTFSGDVQLAQDDIDGIQAIYGRSQNPVQPIGPQTPKACDSKLTFDAITTIRGEVMFFKDRFYMRTNPFYPEVELNFISVFWPQLPNGLEAAYEFADRDEVRFFKGNKYWAVQGQNVLHGYPKDIYSSFGFPRTVKHIDAALSEENTGKTYFFVANKYWRYDEYKRSMDPGYPKMIAHDFPGIGHKVDAVFMKDGFFYFFHGTRQYKFDPKTKRILTLQKANSWFNCRKN

SEQ ID NO: 30 - RNA, 19 nt, synthetic construct
  misc_feature 1..19: note = synthetic construct
  source 1..19: mol_type = other RNA
ttagcttactgtcacacgc

SEQ ID NO: 31 - RNA, 19 nt, synthetic construct
  misc_feature 1..19: note = synthetic construct
  source 1..19: mol_type = other RNA
ttatattcatcatacctcc

SEQ ID NO: 32 - RNA, 19 nt, synthetic construct
  misc_feature 1..19: note = synthetic construct
  source 1..19: mol_type = other RNA
ttgtcttctttctcagtgc

SEQ ID NO: 33 - RNA, 19 nt, synthetic construct
  misc_feature 1..19: note = synthetic construct
  source 1..19: mol_type = other RNA
ttcgtaagcagcttcaagc

SEQ ID NO: 34 - DNA, 31 bp, synthetic construct
  misc_feature 1..31: note = synthetic construct
  source 1..31: mol_type = other DNA
ccggtcttcgtaagcagcttcaagcgaccgg

SEQ ID NO: 35 - DNA, 30 bp, synthetic construct
  misc_feature 1..30: note = synthetic construct
  source 1..30: mol_type = other DNA
ccggtctaaagaacatcactttccgaccgg

SEQ ID NO: 36 - DNA, 31 bp, synthetic construct
  misc_feature 1..31: note = synthetic construct
  source 1..31: mol_type = other DNA
ccggtctaaaacagtagaaacaagggaccgg

SEQ ID NO: 37 - DNA, 2387 bp, Homo sapiens
  source 1..2387: mol_type = unassigned DNA
agacacctctgccctcaccatgagcctctggcagcccctggtcctggtgctcctggtgctgggctgctgctttgctgcccccagacagcgccagtccacccttgtgctcttccctggagacctgagaaccaatctcaccgacaggcagctggcagaggaatacctgtaccgctatggttacactcgggtggcagagatgcgtggagagtcgaaatctctggggcctgcgctgctgcttctccagaagcaactgtccctgcccgagaccggtgagctggatagcgccacgctgaaggccatgcgaaccccacggtgcggggtcccagacctgggcagattccaaacctttgagggcgacctcaagtggcaccaccacaacatcacctattggatccaaaactactcggaagacttgccgcgggcggtgattgacgacgcctttgcccgcgccttcgcactgtggagcgcggtgacgccgctcaccttcactcgcgtgtacagccgggacgcagacatcgtcatccagtttggtgtcgcggagcacggagacgggtatcccttcgacgggaaggacgggctcctggcacacgcctttcctcctggccccggcattcagggagacgcccatttcgacgatgacgagttgtggtccctgggcaagggcgtcgtggttccaactcggtttggaaacgcagatggcgcggcctgccacttccccttcatcttcgagggccgctcctactctgcctgcaccaccgacggtcgctccgacggcttgccctggtgcagtaccacggccaactacgacaccgacgaccggtttggcttctgccccagcgagagactctacacccaggacggcaatgctgatgggaaaccctgccagtttccattcatcttccaaggccaatcctactccgcctgcaccacggacggtcgctccgacggctaccgctggtgcgccaccaccgccaactacgaccgggacaagctcttcggcttctgcccgacccgagctgactcgacggtgatggggggcaactcggcgggggagctgtgcgtcttccccttcactttcctgggtaaggagtactcgacctgtaccagcgagggccgcggagatgggcgcctctggtgcgctaccacctcgaactttgacagcgacaagaagtggggcttctgcccggaccaaggatacagtttgttcctcgtggcggcgcatgagttcggccacgcgctgggcttagatcattcctcagtgccggaggcgctcatgtaccctatgtaccgcttcactgaggggccccccttgcataaggacgacgtgaatggcatccggcacctctatggtcctcgccctgaacctgagccacggcctccaaccaccaccacaccgcagcccacggctcccccgacggtctgccccaccggaccccccactgtccacccctcagagcgccccacagctggccccacaggtcccccctcagctggccccacaggtccccccactgctggcccttctacggccactactgtgcctttgagtccggtggacgatgcctgcaacgtgaacatcttcgacgccatcgcggagattgggaaccagctgtatttgttcaaggatgggaagtactggcgattctctgagggcagggggagccggccgcagggccccttccttatcgccgacaagtggcccgcgctgccccgcaagctggactcggtctttgaggagcggctctccaagaagcttttcttcttctctgggcgccaggtgtgggtgtacacaggcgcgtcggtgctgggcccgaggcgtctggacaagctgggcctgggagccgacgtggcccaggtgaccggggccctccggagtggcagggggaagatgctgctgttcagcgggcggcgcctctggaggttcgacgtgaaggcgcagatggtggatccccggagcgccagcgaggtggaccggatgttccccggggtgcctttggacacgcacgacgtcttccagtaccgagagaaagcctatttctgccaggaccgcttctactggcgcgtgagttcccggagtgagttgaaccaggtggaccaagtgggctacgtgacctatgacatcctgcagtgccctgaggactagggctcccgtcctgctttggcagtgccatgtaaatccccactgggaccaaccctggggaaggagccagtttgccggatacaaactggtattctgttctggaggaaagggaggagtggaggtgggctgggccctctcttctcacctttgttttttgttggagtgtttctaataaacttggattctctaacctttaaaaaaaaaaaaaaaaaaaaaaaaaaaaaaaaaaaaaaaaaaaaaaaaaaaa

SEQ ID NO: 38 - protein, 707 aa, Homo sapiens
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTQDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEERLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 39 - RNA, 21 nt, synthetic construct
  misc_feature 1..21: note = synthetic construct
  source 1..21: mol_type = other RNA
ttgtcgctgtcaaagttcgag

SEQ ID NO: 40 - RNA, 21 nt, synthetic construct
  misc_feature 1..21: note = synthetic construct
  source 1..21: mol_type = other RNA
ttcttgtcgctgtcaaagttc

SEQ ID NO: 41 - RNA, 21 nt, synthetic construct
  misc_feature 1..21: note = synthetic construct
  source 1..21: mol_type = other RNA
ttcaactcactccgggaactc

SEQ ID NO: 42 - RNA, 21 nt, synthetic construct
  misc_feature 1..21: note = synthetic construct
  source 1..21: mol_type = other RNA
ttcacgtcgtccttatgcaag

SEQ ID NO: 43 - DNA, 31 bp, synthetic construct
  misc_feature 1..31: note = synthetic construct
  source 1..31: mol_type = other DNA
ccggtcttgtcgctgtcaaagttcggaccgg

SEQ ID NO: 44 - DNA, 31 bp, synthetic construct
  misc_feature 1..31: note = synthetic construct
  source 1..31: mol_type = other DNA
ccggtcttattagaaacactccaacgaccgg

SEQ ID NO: 45 - DNA, 31 bp, synthetic construct
  misc_feature 1..31: note = synthetic construct
  source 1..31: mol_type = other DNA
ccggtcattcacgtcgtccttatgcgaccgg

SEQ ID NO: 46 - DNA, 1828 bp, Homo sapiens
  source 1..1828: mol_type = unassigned DNA
ctacaaggaggcaggcaagacagcaaggcatagagacaacatagagctaagtaaagccagtggaaatgaagagtcttccaatcctactgttgctgtgcgtggcagtttgctcagcctatccattggatggagctgcaaggggtgaggacaccagcatgaaccttgttcagaaatatctagaaaactactacgacctcaaaaaagatgtgaaacagtttgttaggagaaaggacagtggtcctgttgttaaaaaaatccgagaaatgcagaagttccttggattggaggtgacggggaagctggactccgacactctggaggtgatgcgcaagcccaggtgtggagttcctgatgttggtcacttcagaacctttcctggcatcccgaagtggaggaaaacccaccttacatacaggattgtgaattatacaccagatttgccaaaagatgctgttgattctgctgttgagaaagctctgaaagtctgggaagaggtgactccactcacattctccaggctgtatgaaggagaggctgatataatgatctcttttgcagttagagaacatggagacttttacccttttgatggacctggaaatgttttggcccatgcctatgcccctgggccagggattaatggagatgcccactttgatgatgatgaacaatggacaaaggatacaacagggaccaatttatttctcgttgctgctcatgaaattggccactccctgggtctctttcactcagccaacactgaagctttgatgtacccactctatcactcactcacagacctgactcggttccgcctgtctcaagatgatataaatggcattcagtccctctatggacctccccctgactcccctgagacccccctggtacccacggaacctgtccctccagaacctgggacgccagccaactgtgatcctgctttgtcctttgatgctgtcagcactctgaggggagaaatcctgatctttaaagacaggcacttttggcgcaaatccctcaggaagcttgaacctgaattgcatttgatctcttcattttggccatctcttccttcaggcgtggatgccgcatatgaagttactagcaaggacctcgttttcatttttaaaggaaatcaattctgggctatcagaggaaatgaggtacgagctggatacccaagaggcatccacaccctaggtttccctccaaccgtgaggaaaatcgatgcagccatttctgataaggaaaagaacaaaacatatttctttgtagaggacaaatactggagatttgatgagaagagaaattccatggagccaggctttcccaagcaaatagctgaagactttccagggattgactcaaagattgatgctgtttttgaagaatttgggttcttttatttctttactggatcttcacagttggagtttgacccaaatgcaaagaaagtgacacacactttgaagagtaacagctggcttaattgttgaaagagatatgtagaaggcacaatatgggcactttaaatgaagctaataattcttcacctaagtctctgtgaattgaaatgttcgttttctcctgcctgtgctgtgactcgagtcacactcaagggaacttgagcgtgaatctgtatcttgccggtcatttttatgttattacagggcattcaaatgggctgctgcttagcttgcaccttgtcacatagagtgatctttcccaagagaaggggaagcactcgtgtgcaacagacaagtgactgtatctgtgtagactatttgcttatttaataaagacgatttgtcagttattttatctt

SEQ ID NO: 47 - protein, 477 aa, Homo sapiens
MKSLPILLLLCVAVCSAYPLDGAARGEDTSMNLVQKYLENYYDLKKDVKQFVRRKDSGPVVKKIREMQKFLGLEVTGKLDSDTLEVMRKPRCGVPDVGHFRTFPGIPKWRKTHLTYRIVNYTPDLPKDAVDSAVEKALKVWEEVTPLTFSRLYEGEADIMISFAVREHGDFYPFDGPGNVLAHAYAPGPGINGDAHFDDDEQWTKDTTGTNLFLVAAHEIGHSLGLFHSANTEALMYPLYHSLTDLTRFRLSQDDINGIQSLYGPPPDSPETPLVPTEPVPPEPGTPANCDPALSFDAVSTLRGEILIFKDRHFWRKSLRKLEPELHLISSFWPSLPSGVDAAYEVTSKDLVFIFKGNQFWAIRGNEVRAGYPRGIHTLGFPPTVRKIDAAISDKEKNKTYFFVEDKYWRFDEKRNSMEPGFPKQIAEDFPGIDSKIDAVFEEFGFFYFFTGSSQLEFDPNAKKVTHTLKSNSWLNC

SEQ ID NO: 48 - RNA, 20 nt, synthetic construct
  misc_feature 1..20: note = synthetic construct
  source 1..20: mol_type = other RNA
ttcatcatcatcaaagtggg

SEQ ID NO: 49 - RNA, 20 nt, synthetic construct
  misc_feature 1..20: note = synthetic construct
  source 1..20: mol_type = other RNA
taataacataaaaatgaccg

SEQ ID NO: 50 - RNA, 20 nt, synthetic construct
  misc_feature 1..20: note = synthetic construct
  source 1..20: mol_type = other RNA
tagtctacacagatacagtc

SEQ ID NO: 51 - RNA, 20 nt, synthetic construct
  misc_feature 1..20: note = synthetic construct
  source 1..20: mol_type = other RNA
tatatcatcttgagacaggc

SEQ ID NO: 52 - DNA, 32 bp, synthetic construct
  misc_feature 1..32: note = synthetic construct
  source 1..32: mol_type = other DNA
ccggtctatatcatcttgagacaggcgaccgg

SEQ ID NO: 53 - DNA, 32 bp, synthetic construct
  misc_feature 1..32: note = synthetic construct
  source 1..32: mol_type = other DNA
ccggtctttctcttctcatcaaatctgaccgg

SEQ ID NO: 54 - DNA, 32 bp, synthetic construct
  misc_feature 1..32: note = synthetic construct
  source 1..32: mol_type = other DNA
ccggtctaacaaactgtttcacatctgaccgg

SEQ ID NO: 55 - DNA, 2943 bp, Homo sapiens
  source 1..2943: mol_type = unassigned DNA
accaggcaacaccattgaaggctcatatgtaaaaatccatgccttcctttctcccaatctccattcccaaacttagccactggcttctggctgaggccttacgcatacctcccggggcttgcacacaccttcttctacagaagacacaccttgggcatatcctacagaagaccaggcttctctctggtccttggtagagggctactttactgtaacagggccagggtggagagttctctcctgaagctccatcccctctataggaaatgtgttgacaatattcagaagagtaagaggatcaagacttctttgtgctcaaataccactgttctcttctctaccctgccctaaccaggagcttgtcaccccaaactctgaggtgatttatgccttaatcaagcaaacttccctcttcagaaaagatggctcattttccctcaaaagttgccaggagctgccaagtattctgccaattcaccctggagcacaatcaacaaattcagccagaacacaactacagctactattagaactattattattaataaattcctctccaaatctagccccttgacttcggatttcacgatttctcccttcctcctagaaacttgataagtttcccgcgcttccctttttctaagactacatgtttgtcatcttataaagcaaaggggtgaataaatgaaccaaatcaataacttctggaatatctgcaaacaacaataatatcagctatgccatctttcactattttagccagtatcgagttgaatgaacatagaaaaatacaaaactgaattcttccctgtaaattccccgttttgacgacgcacttgtagccacgtagccacgcctacttaagacaattacaaaaggcgaagaagactgactcaggcttaagctgccagccagagagggagtcatttcattggcgtttgagtcagcaaagaagtcaagatggccaaagttccagacatgtttgaagacctgaagaactgttacagtgaaaatgaagaagacagttcctccattgatcatctgtctctgaatcagaaatccttctatcatgtaagctatggcccactccatgaaggctgcatggatcaatctgtgtctctgagtatctctgaaacctctaaaacatccaagcttaccttcaaggagagcatggtggtagtagcaaccaacgggaaggttctgaagaagagacggttgagtttaagccaatccatcactgatgatgacctggaggccatcgccaatgactcagaggaagaaatcatcaagcctaggtcagcaccttttagcttcctgagcaatgtgaaatacaactttatgaggatcatcaaatacgaattcatcctgaatgacgccctcaatcaaagtataattcgagccaatgatcagtacctcacggctgctgcattacataatctggatgaagcagtgaaatttgacatgggtgcttataagtcatcaaaggatgatgctaaaattaccgtgattctaagaatctcaaaaactcaattgtatgtgactgcccaagatgaagaccaaccagtgctgctgaaggagatgcctgagatacccaaaaccatcacaggtagtgagaccaacctcctcttcttctgggaaactcacggcactaagaactatttcacatcagttgcccatccaaacttgtttattgccacaaagcaagactactgggtgtgcttggcaggggggccaccctctatcactgactttcagatactggaaaaccaggcgtaggtctggagtctcacttgtctcacttgtgcagtgttgacagttcatatgtaccatgtacatgaagaagctaaatcctttactgttagtcatttgctgagcatgtactgagccttgtaattctaaatgaatgtttacactctttgtaagagtggaaccaacactaacatataatgttgttatttaaagaacaccctatattttgcatagtaccaatcattttaattattattcttcataacaattttaggaggaccagagctactgactatggctaccaaaaagactctacccatattacagatgggcaaattaaggcataagaaaactaagaaatatgcacaatagcagttgaaacaagaagccacagacctaggatttcatgatttcatttcaactgtttgccttctacttttaagttgctgatgaactcttaatcaaatagcataagtttctgggacctcagttttatcattttcaaaatggagggaataatacctaagccttcctgccgcaacagttttttatgctaatcagggaggtcattttggtaaaatacttcttgaagccgagcctcaagatgaaggcaaagcacgaaatgttattttttaattattatttatatatgtatttataaatatatttaagataattataatatactatatttatgggaaccccttcatcctctgagtgtgaccaggcatcctccacaatagcagacagtgttttctgggataagtaagtttgatttcattaatacagggcattttggtccaagttgtgcttatcccatagccaggaaactctgcattctagtacttgggagacctgtaatcatataataaatgtacattaattaccttgagccagtaattggtccgatctttgactcttttgccattaaacttacctgggcattcttgtttcaattccacctgcaatcaagtcctacaagctaaaattagatgaactcaactttgacaaccatgagaccactgttatcaaaactttcttttctggaatgtaatcaatgtttcttctaggttctaaaaattgtgatcagaccataatgttacattattatcaacaatagtgattgatagagtgttatcagtcataactaaataaagcttgcaacaaaattctctgacaaaaaaaaaaaaaaaa

SEQ ID NO: 56 - protein, 271 aa, Homo sapiens
MAKVPDMFEDLKNCYSENEEDSSSIDHLSLNQKSFYHVSYGPLHEGCMDQSVSLSISETSKTSKLTFKESMVVVATNGKVLKKRRLSLSQSITDDDLEAIANDSEEEIIKPRSAPFSFLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 57 - RNA, 20 nt, synthetic construct
  misc_feature 1..20: note = synthetic construct
  source 1..20: mol_type = other RNA
tttctatgttcattcaactc

SEQ ID NO: 58 - RNA, 20 nt, synthetic construct
  misc_feature 1..20: note = synthetic construct
  source 1..20: mol_type = other RNA
tcattcaactcgatactggc

SEQ ID NO: 59 - RNA, 20 nt, synthetic construct
  misc_feature 1..20: note = synthetic construct
  source 1..20: mol_type = other RNA
ttcattcaactcgatactgg

SEQ ID NO: 60 - RNA, 20 nt, synthetic construct
  misc_feature 1..20: note = synthetic construct
  source 1..20: mol_type = other RNA
taatagttctaatagtagct

SEQ ID NO: 61 - DNA, 32 bp, synthetic construct
  misc_feature 1..32: note = synthetic construct
  source 1..32: mol_type = other DNA
ccggtctttcttagttttcttatgccgaccgg

SEQ ID NO: 62 - DNA, 31 bp, synthetic construct
  misc_feature 1..31: note = synthetic construct
  source 1..31: mol_type = other DNA
ccggtctaatagttctaatagtagcgaccgg

SEQ ID NO: 63 - DNA, 31 bp, synthetic construct
  misc_feature 1..31: note = synthetic construct
  source 1..31: mol_type = other DNA
ccggtctatgaactgtcaacactgcgaccgg

SEQ ID NO: 64 - DNA, 1498 bp, Homo sapiens
  source 1..1498: mol_type = unassigned DNA
accaaacctcttcgaggcacaaggcacaacaggctgctctgggattctcttcagccaatcttcattgctcaagtgtctgaagcagccatggcagaagtacctgagctcgccagtgaaatgatggcttattacagtggcaatgaggatgacttgttctttgaagctgatggccctaaacagatgaagtgctccttccaggacctggacctctgccctctggatggcggcatccagctacgaatctccgaccaccactacagcaagggcttcaggcaggccgcgtcagttgttgtggccatggacaagctgaggaagatgctggttccctgcccacagaccttccaggagaatgacctgagcaccttctttcccttcatctttgaagaagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaaagagagctgtacccagagagtcctgtgctgaatgtggactcaatccctagggctggcagaaagggaacagaaaggtttttgagtacggctatagcctggactttcctgttgtctacaccaatgcccaactgcctgccttagggtagtgctaagaggatctcctgtccatcagccaggacagtcagctctctcctttcagggccaatccccagcccttttgttgagccaggcctctctcacctctcctactcacttaaagcccgcctgacagaaaccacggccacatttggttctaagaaaccctctgtcattcgctcccacattctgatgagcaaccgcttccctatttatttatttatttgtttgtttgttttattcattggtctaatttattcaaagggggcaagaagtagcagtgtctgtaaaagagcctagtttttaatagctatggaatcaattcaatttggactggtgtgctctctttaaatcaagtcctttaattaagactgaaaatatataagctcagattatttaaatgggaatatttataaatgagcaaatatcatactgttcaatggttctgaaataaacttcactgaag

SEQ ID NO: 65 - protein, 269 aa, Homo sapiens
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 66 - RNA, 21 nt, synthetic construct
  misc_feature 1..21: note = synthetic construct
  source 1..21: mol_type = other RNA
ttatcatctttcaacacgcag

SEQ ID NO: 67 - RNA, 21 nt, synthetic construct
  misc_feature 1..21: note = synthetic construct
  source 1..21: mol_type = other RNA
ttttacagacactgctacttc

SEQ ID NO: 68 - RNA, 21 nt, synthetic construct
  misc_feature 1..21: note = synthetic construct
  source 1..21: mol_type = other RNA
tttgtcattactttcttctcc

SEQ ID NO: 69 - RNA, 21 nt, synthetic construct
  misc_feature 1..21: note = synthetic construct
  source 1..21: mol_type = other RNA
tacagacactgctacttcttg

SEQ ID NO: 70 - DNA, 33 bp, synthetic construct
  misc_feature 1..33: note = synthetic construct
  source 1..33: mol_type = other DNA
ccggtcttttgtcattactttcttctcgaccgg

SEQ ID NO: 71 - DNA, 33 bp, synthetic construct
  misc_feature 1..33: note = synthetic construct
  source 1..33: mol_type = other DNA
ccggtctttcagtcttaattaaaggacgaccgg

SEQ ID NO: 72 - DNA, 32 bp, synthetic construct
  misc_feature 1..32: note = synthetic construct
  source 1..32: mol_type = other DNA
ccggtcttacataaattaactcagctgaccgg

SEQ ID NO: 73 - DNA, 1201 bp, Homo sapiens
  source 1..1201: mol_type = unassigned DNA
aatattagagtctcaacccccaataaatataggactggagatgtctgaggctcattctgccctcgagcccaccgggaacgaaagagaagctctatctcccctccaggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattcagcaaaaaaaaaaaaaaaaaaa

SEQ ID NO: 74 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 75 - RNA, 21 nt, synthetic construct
  misc_feature 1..21: note = synthetic construct
  source 1..21: mol_type = other RNA
taaaatagtgtcctaacgctc

SEQ ID NO: 76 - RNA, 21 nt, synthetic construct
  misc_feature 1..21: note = synthetic construct
  source 1..21: mol_type = other RNA
tcactactctcaaatctgttc

SEQ ID NO: 77 - RNA, 21 nt, synthetic construct
  misc_feature 1..21: note = synthetic construct
  source 1..21: mol_type = other RNA
ttactcttgttacatgtctcc

SEQ ID NO: 78 - RNA, 21 nt, synthetic construct
  misc_feature 1..21: note = synthetic construct
  source 1..21: mol_type = other RNA
taacgctcatacttttagttc

SEQ ID NO: 79 - DNA, 33 bp, synthetic construct
  misc_feature 1..33: note = synthetic construct
  source 1..33: mol_type = other DNA
ccggtcttactcttgttacatgtcyccgacctt

SEQ ID NO: 80 - DNA, 33 bp, synthetic construct
  misc_feature 1..33: note = synthetic construct
  source 1..33: mol_type = other DNA
ccggtcttactcttgttacatgtctccgacctt

SEQ ID NO: 81 - DNA, 33 bp, synthetic construct
  misc_feature 1..33: note = synthetic construct
  source 1..33: mol_type = other DNA
ccggtctacataaaatgtttcaagtgggacctt

SEQ ID NO: 82 - DNA, 1718 bp, Homo sapiens
  source 1..1718: mol_type = unassigned DNA
gagggtgcataagttctctagtagggtgatgatataaaaagccaccggagcactccataaggcacaaactttcagagacagcagagcacacaagcttctaggacaagagccaggaagaaaccaccggaaggaaccatctcactgtgtgtaaacatgacttccaagctggccgtggctctcttggcagccttcctgatttctgcagctctgtgtgaaggtgcagttttgccaaggagtgctaaagaacttagatgtcagtgcataaagacatactccaaacctttccaccccaaatttatcaaagaactgagagtgattgagagtggaccacactgcgccaacacagaaattattgtaaagctttctgatggaagagagctctgtctggaccccaaggaaaactgggtgcagagggttgtggagaagtttttgaagagggctgagaattcataaaaaaattcattctctgtggtatccaagaatcagtgaagatgccagtgaaacttcaagcaaatctacttcaacacttcatgtattgtgtgggtctgttgtagggttgccagatgcaatacaagattcctggttaaatttgaatttcagtaaacaatgaatagtttttcattgtaccatgaaatatccagaacatacttatatgtaaagtattatttatttgaatctacaaaaaacaacaaataatttttaaatataaggattttcctagatattgcacgggagaatatacaaatagcaaaattgaggccaagggccaagagaatatccgaactttaatttcaggaattgaatgggtttgctagaatgtgatatttgaagcatcacataaaaatgatgggacaataaattttgccataaagtcaaatttagctggaaatcctggatttttttctgttaaatctggcaaccctagtctgctagccaggatccacaagtccttgttccactgtgccttggtttctcctttatttctaagtggaaaaagtattagccaccatcttacctcacagtgatgttgtgaggacatgtggaagcactttaagttttttcatcataacataaattattttcaagtgtaacttattaacctatttattatttatgtatttatttaagcatcaaatatttgtgcaagaatttggaaaaatagaagatgaatcattgattgaatagttataaagatgttatagtaaatttattttattttagatattaaatgatgttttattagataaatttcaatcagggtttttagattaaacaaacaaacaattgggtacccagttaaattttcatttcagataaacaacaaataattttttagtataagtacattattgtttatctgaaattttaattgaactaacaatcctagtttgatactcccagtcttgtcattgccagctgtgttggtagtgctgtgttgaattacggaataatgagttagaactattaaaacagccaaaactccacagtcaatattagtaatttcttgctggttgaaacttgtttattatgtacaaatagattcttataatattatttaaatgactgcatttttaaatacaaggctttatatttttaactttaagatgtttttatgtgctctccaaattttttttactgtttctgattgtatggaaatataaaagtaaatatgaaacatttaaaatataatttgttgtcaaagtaaaaaaaaaaaaaaa

SEQ ID NO: 83 - protein, 99 aa, Homo sapiens
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 84 - RNA, 20 nt, synthetic construct
  misc_feature 1..20: note = synthetic construct
  source 1..20: mol_type = other RNA
tttgtttaatctaaaaaccc

SEQ ID NO: 85 - RNA, 20 nt, synthetic construct
  misc_feature 1..20: note = synthetic construct
  source 1..20: mol_type = other RNA
tttacacacagtgagatggt

SEQ ID NO: 86 - RNA, 20 nt, synthetic construct
  misc_feature 1..20: note = synthetic construct
  source 1..20: mol_type = other RNA
ttcaaatatcacattctagc

SEQ ID NO: 87 - RNA, 20 nt, synthetic construct
  misc_feature 1..20: note = synthetic construct
  source 1..20: mol_type = other RNA
ttatgcactgacatctaagt

SEQ ID NO: 88 - DNA, 32 bp, synthetic construct
  misc_feature 1..32: note = synthetic construct
  source 1..32: mol_type = other DNA
ccggtctatcacattctagcaaacccgaccgg

SEQ ID NO: 89 - DNA, 32 bp, synthetic construct
  misc_feature 1..32: note = synthetic construct
  source 1..32: mol_type = other DNA
ccggtctactagagaacttatgcaccgaccgg

SEQ ID NO: 90 - DNA, 32 bp, synthetic construct
  misc_feature 1..32: note = synthetic construct
  source 1..32: mol_type = other DNA
ccggtctagttctaactcattattccgaccgg

SEQ ID NO: 91 - DNA, 5170 bp, Homo sapiens
  source 1..5170: mol_type = unassigned DNA
gtggccggcggccggagccgactcggagcgcgcggcgccggccgggaggagccggagagcggccgggccgggcggtgggggcgccggcctgccccgcgcgccccagggagcggcaggaatgtgacaatcgcgcgcccgcgcaccgaagcactcctcgctcggctcctagggctctcgcccctctgagctgagccgggttccgcccggggctgggatcccatcaccctccacggccgtccgtccaggtagacgcaccctctgaagatggtgactccctcctgagaagctggaccccttggtaaaagacaaggccttctccaagaagaatatgaaagtgttactcagacttatttgtttcatagctctactgatttcttctctggaggctgataaatgcaaggaacgtgaagaaaaaataattttagtgtcatctgcaaatgaaattgatgttcgtccctgtcctcttaacccaaatgaacacaaaggcactataacttggtataaagatgacagcaagacacctgtatctacagaacaagcctccaggattcatcaacacaaagagaaactttggtttgttcctgctaaggtggaggattcaggacattactattgcgtggtaagaaattcatcttactgcctcagaattaaaataagtgcaaaatttgtggagaatgagcctaacttatgttataatgcacaagccatatttaagcagaaactacccgttgcaggagacggaggacttgtgtgcccttatatggagttttttaaaaatgaaaataatgagttacctaaattacagtggtataaggattgcaaacctctacttcttgacaatatacactttagtggagtcaaagataggctcatcgtgatgaatgtggctgaaaagcatagagggaactatacttgtcatgcatcctacacatacttgggcaagcaatatcctattacccgggtaatagaatttattactctagaggaaaacaaacccacaaggcctgtgattgtgagcccagctaatgagacaatggaagtagacttgggatcccagatacaattgatctgtaatgtcaccggccagttgagtgacattgcttactggaagtggaatgggtcagtaattgatgaagatgacccagtgctaggggaagactattacagtgtggaaaatcctgcaaacaaaagaaggagtaccctcatcacagtgcttaatatatcggaaattgaaagtagattttataaacatccatttacctgttttgccaagaatacacatggtatagatgcagcatatatccagttaatatatccagtcactaatttccagaagcacatgattggtatatgtgtcacgttgacagtcataattgtgtgttctgttttcatctataaaatcttcaagattgacattgtgctttggtacagggattcctgctatgattttctcccaataaaagcttcagatggaaagacctatgacgcatatatactgtatccaaagactgttggggaagggtctacctctgactgtgatatttttgtgtttaaagtcttgcctgaggtcttggaaaaacagtgtggatataagctgttcatttatggaagggatgactacgttggggaagacattgttgaggtcattaatgaaaacgtaaagaaaagcagaagactgattatcattttagtcagagaaacatcaggcttcagctggctgggtggttcatctgaagagcaaatagccatgtataatgctcttgttcaggatggaattaaagttgtcctgcttgagctggagaaaatccaagactatgagaaaatgccagaatcgattaaattcattaagcagaaacatggggctatccgctggtcaggggactttacacagggaccacagtctgcaaagacaaggttctggaagaatgtcaggtaccacatgccagtccagcgacggtcaccttcatctaaacaccagttactgtcaccagccactaaggagaaactgcaaagagaggctcacgtgcctctcgggtagcatggagaagttgccaagagttctttaggtgcctcctgtcttatggcgttgcaggccaggttatgcctcatgctgacttgcagagttcatggaatgtaactatatcatcctttatccctgaggtcacctggaatcagattattaagggaataagccatgacgtcaatagcagcccagggcacttcagagtagagggcttgggaagatcttttaaaaaggcagtaggcccggtgtggtggctcacgcctataatcccagcactttgggaggctgaagtgggtggatcaccagaggtcaggagttcgagaccagcccagccaacatggcaaaaccccatctctactaaaaatacaaaaatgagctaggcatggtggcacacgcctgtaatcccagctacacctgaggctgaggcaggagaattgcttgaaccggggagacggaggttgcagtgagccgagtttgggccactgcactctagcctggcaacagagcaagactccgtctcaaaaaaagggcaataaatgccctctctgaatgtttgaactgccaagaaaaggcatggagacagcgaactagaagaaagggcaagaaggaaatagccaccgtctacagatggcttagttaagtcatccacagcccaagggcggggctatgccttgtctggggaccctgtagagtcactgaccctggagcggctctcctgagaggtgctgcaggcaaagtgagactgacacctcactgaggaagggagacatattcttggagaactttccatctgcttgtattttccatacacatccccagccagaagttagtgtccgaagaccgaattttattttacagagcttgaaaactcacttcaatgaacaaagggattctccaggattccaaagttttgaagtcatcttagctttccacaggagggagagaacttaaaaaagcaacagtagcagggaattgatccacttcttaatgctttcctccctggcatgaccatcctgtcctttgttattatcctgcattttacgtctttggaggaacagctccctagtggcttcctccgtctgcaatgtcccttgcacagcccacacatgaaccatccttcccatgatgccgctcttctgtcatcccgctcctgctgaaacacctcccaggggctccacctgttcaggagctgaagcccatgctttcccaccagcatgtcactcccagaccacctccctgccctgtcctccagcttcccctcgctgtcctgctgtgtgaattcccaggttggcctggtggccatgtcgcctgcccccagcactcctctgtctctgctcttgcctgcacccttcctcctcctttgcctaggaggccttctcgcattttctctagctgatcagaattttaccaaaattcagaacatcctccaattccacagtctctgggagactttccctaagaggcgacttcctctccagccttctctctctggtcaggcccactgcagagatggtggtgagcacatctgggaggctggtctccctccagctggaattgctgctctctgagggagaggctgtggtggctgtctctgtccctcactgccttccaggagcaatttgcacatgtaacatagatttatgtaatgctttatgtttaaaaacattccccaattatcttatttaatttttgcaattattctaattttatatatagagaaagtgacctattttttaaaaaaatcacactctaagttctattgaacctaggacttgagcctccatttctggcttctagtctggtgttctgagtacttgatttcaggtcaataacggtcccccctcactccacactggcacgtttgtgagaagaaatgacattttgctaggaagtgaccgagtctaggaatgcttttattcaagacaccaaattccaaacttctaaatgttggaattttcaaaaattgtgtttagattttatgaaaaactcttctactttcatctattctttccctagaggcaaacatttcttaaaatgtttcattttcattaaaaatgaaagccaaatttatatgccaccgattgcaggacacaagcacagttttaagagttgtatgaacatggagaggacttttggtttttatatttctcgtatttaatatgggtgaacaccaacttttatttggaataataattttcctcctaaacaaaaacacattgagtttaagtctctgactcttgcctttccacctgctttctcctgggcccgctttgcctgcttgaaggaacagtgctgttctggagctgctgttccaacagacagggcctagctttcatttgacacacagactacagccagaagcccatggagcagggatgtcacgtcttgaaaagcctattagatgttttacaaatttaattttgcagattattttagtctgtcatccagaaaatgtgtcagcatgcatagtgctaagaaagcaagccaatttggaaacttaggttagtgacaaaattggccagagagtgggggtgatgatgaccaagaattacaagtagaatggcagctggaatttaaggagggacaagaatcaatggataagcgtgggtggaggaagatccaaacagaaaagtgcaaagttattccccatcttccaagggttgaattctggaggaagaagacacattcctagttccccgtgaacttcctttgacttattgtccccactaaaacaaaacaaaaaacttttaatgccttccacattaattagattttcttgcagtttttttatggcatttttttaaagatgccctaagtgttgaagaagagtttgcaaatgcaacaaaatatttaattaccggttgttaaaactggtttagcacaatttatattttccctctcttgcctttcttatttgcaataaaaggtattgagccattttttaaatgacatttttgataaattatgtttgtactagttgatgaaggagttttttttaacctgtttatataattttgcagcagaagccaaattttttgtatattaaagcaccaaattcatgtacagcatgcatcacggatcaatagactgtacttattttccaataaaattttcaaactttgtactgttaaaaaaaaaaaaaaaaaa

SEQ ID NO: 92 - protein, 569 aa, Homo sapiens
MKVLLRLICFIALLISSLEADKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVTNFQKHMIGICVTLTVIIVCSVFIYKIFKIDIVLWYRDSCYDFLPIKASDGKTYDAYILYPKTVGEGSTSDCDIFVFKVLPEVLEKQCGYKLFIYGRDDYVGEDIVEVINENVKKSRRLIIILVRETSGFSWLGGSSEEQIAMYNALVQDGIKVVLLELEKIQDYEKMPESIKFIKQKHGAIRWSGDFTQGPQSAKTRFWKNVRYHMPVQRRSPSSKHQLLSPATKEKLQREAHVPLG

SEQ ID NO: 93 - RNA, 20 nt, synthetic construct
  misc_feature 1..20: note = synthetic construct
  source 1..20: mol_type = other RNA
tttcttctcacaaacgtgcc

SEQ ID NO: 94 - RNA, 20 nt, synthetic construct
  misc_feature 1..20: note = synthetic construct
  source 1..20: mol_type = other RNA
ttataccaagttatagtgcc

SEQ ID NO: 95 - RNA, 20 nt, synthetic construct
  misc_feature 1..20: note = synthetic construct
  source 1..20: mol_type = other RNA
ttgtaaaacatctaataggc

SEQ ID NO: 96 - RNA, 20 nt, synthetic construct
  misc_feature 1..20: note = synthetic construct
  source 1..20: mol_type = other RNA
tttccacactgtaatagtct

SEQ ID NO: 97 - DNA, 31 bp, synthetic construct
  misc_feature 1..31: note = synthetic construct
  source 1..31: mol_type = other DNA
ccggtctttcttctcacaaacgtgcgaccgg

SEQ ID NO: 98 - DNA, 31 bp, synthetic construct
  misc_feature 1..31: note = synthetic construct
  source 1..31: mol_type = other DNA
ccggtcttaaacacaaaaatatcacgaccgg

SEQ ID NO: 99 - DNA, 31 bp, synthetic construct
  misc_feature 1..31: note = synthetic construct
  source 1..31: mol_type = other DNA
ccggtctttccacactgtaatagtcgaccgg

SEQ ID NO: 100 - DNA, 1686 bp, Homo sapiens
  source 1..1686: mol_type = unassigned DNA
cagacgctccctcagcaaggacagcagaggaccagctaagagggagagaagcaactacagaccccccctgaaaacaaccctcagacgccacatcccctgacaagctgccaggcaggttctcttcctctcacatactgacccacggctccaccctctctcccctggaaaggacaccatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaggaggacgaacatccaaccttcccaaacgcctcccctgccccaatccctttattaccccctccttcagacaccctcaacctcttctggctcaaaaagagaattgggggcttagggtcggaacccaagcttagaactttaagcaacaagaccaccacttcgaaacctgggattcaggaatgtgtggcctgcacagtgaagtgctggcaaccactaagaattcaaactggggcctccagaactcactggggcctacagctttgatccctgacatctggaatctggagaccagggagcctttggttctggccagaatgctgcaggacttgagaagacctcacctagaaattgacacaagtggaccttaggccttcctctctccagatgtttccagacttccttgagacacggagcccagccctccccatggagccagctccctctatttatgtttgcacttgtgattatttattatttatttattatttatttatttacagatgaatgtatttatttgggagaccggggtatcctgggggacccaatgtaggagctgccttggctcagacatgttttccgtgaaaacggagctgaacaataggctgttcccatgtagccccctggcctctgtgccttcttttgattatgttttttaaaatatttatctgattaagttgtctaaacaatgctgatttggtgaccaactgtcactcattgctgagcctctgctccccaggggagttgtgtctgtaatcgccctactattcagtggcgagaaataaagtttgcttagaaaagaaaaaaaaaaaaa

SEQ ID NO: 101 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 102 - RNA, 19 nt, synthetic construct
  misc_feature 1..19: note = synthetic construct
  source 1..19: mol_type = other RNA
aataaataatcacaagtgc

SEQ ID NO: 103 - RNA, 19 nt, synthetic construct
  misc_feature 1..19: note = synthetic construct
  source 1..19: mol_type = other RNA
taaaaaacataatcaaaag

SEQ ID NO: 104 - RNA, 19 nt, synthetic construct
  misc_feature 1..19: note = synthetic construct
  source 1..19: mol_type = other RNA
taataaataatcacaagtg

SEQ ID NO: 105 - RNA, 19 nt, synthetic construct
  misc_feature 1..19: note = synthetic construct
  source 1..19: mol_type = other RNA
ttttcttttctaagcaaac

SEQ ID NO: 106 - DNA, 31 bp, synthetic construct
  misc_feature 1..31: note = synthetic construct
  source 1..31: mol_type = other DNA
ccggtcaaacataatcaaaagaagggaccgg

SEQ ID NO: 107 - DNA, 31 bp, synthetic construct
  misc_feature 1..31: note = synthetic construct
  source 1..31: mol_type = other DNA
ccggtctaaaaaacataatcaaaaggaccgg

SEQ ID NO: 108 - DNA, 31 bp, synthetic construct
  misc_feature 1..31: note = synthetic construct
  source 1..31: mol_type = other DNA
ccggtctattttaaaaaacataatcgaccgg

SEQ ID NO: 109 - DNA, 3677 bp, Homo sapiens
  source 1..3677: mol_type = unassigned DNA
tcgcggaggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgtgtacgttggtgcccgctgctgtctaatgccctggagcctccctggcccccatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaaaaaaaaaaaaaaaaa

SEQ ID NO: 110 - protein, 412 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 111 - RNA, 21 nt, synthetic construct
  misc_feature 1..21: note = synthetic construct
  source 1..21: mol_type = other RNA
taaaactctctaatcttccgg

SEQ ID NO: 112 - RNA, 21 nt, synthetic construct
  misc_feature 1..21: note = synthetic construct
  source 1..21: mol_type = other RNA
ttccttctcttcttcctcctc

SEQ ID NO: 113 - RNA, 21 nt, synthetic construct
  misc_feature 1..21: note = synthetic construct
  source 1..21: mol_type = other RNA
tatacacacaaatacaagttg

SEQ ID NO: 114 - RNA, 21 nt, synthetic construct
  misc_feature 1..21: note = synthetic construct
  source 1..21: mol_type = other RNA
ttaaaacgagaaacaatacag

SEQ ID NO: 115 - DNA, 31 bp, synthetic construct
  misc_feature 1..31: note = synthetic construct
  source 1..31: mol_type = other DNA
ccggtctaaaactctctaatcttccgaccgg

SEQ ID NO: 116 - DNA, 31 bp, synthetic construct
  misc_feature 1..31: note = synthetic construct
  source 1..31: mol_type = other DNA
ccggtctttgatccgcataatctgcgaccgg

SEQ ID NO: 117 - DNA, 31 bp, synthetic construct
  misc_feature 1..31: note = synthetic construct
  source 1..31: mol_type = other DNA
ccggtcttgaaattaaatattaaccgaccgg

SEQ ID NO: 118 - DNA, 2583 bp, Homo sapiens
  source 1..2583: mol_type = unassigned DNA
agccggtccccgccgccgccgcccttcgcgccctgggccatctccctcccacctccctccgcggagcagccagacagcgagggccccggccgggggcaggggggacgccccgtccggggcacccccccggctctgagccgcccgcggggccggcctcggcccggagcggaggaaggagtcgccgaggagcagcctgaggccccagagtctgagacgagccgccgccgcccccgccactgcggggaggagggggaggaggagcgggaggagggacgagctggtcgggagaagaggaaaaaaacttttgagacttttccgttgccgctgggagccggaggcgcggggacctcttggcgcgacgctgccccgcgaggaggcaggacttggggaccccagaccgcctccctttgccgccggggacgcttgctccctccctgccccctacacggcgtccctcaggcgcccccattccggaccagccctcgggagtcgccgacccggcctcccgcaaagacttttccccagacctcgggcgcaccccctgcacgccgccttcatccccggcctgtctcctgagcccccgcgcatcctagaccctttctcctccaggagacggatctctctccgacctgccacagatcccctattcaagaccacccaccttctggtaccagatcgcgcccatctaggttatttccgtgggatactgagacacccccggtccaagcctcccctccaccactgcgcccttctccctgaggacctcagctttccctcgaggccctcctaccttttgccgggagacccccagcccctgcaggggcggggcctccccaccacaccagccctgttcgcgctctcggcagtgccggggggcgccgcctcccccatgccgccctccgggctgcggctgctgccgctgctgctaccgctgctgtggctactggtgctgacgcctggccggccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagctgaggtcccgccccgccccgccccgccccggcaggcccggccccaccccgccccgcccccgctgccttgcccatgggggctgtatttaaggacacccgtgccccaagcccacctggggccccattaaagatggagagaggactgcggatctctgtgtcattgggcgcctgcctggggtctccatccctgacgttcccccactcccactccctctctctccctctctgcctcctcctgcctgtctgcactattcctttgcccggcatcaaggcacaggggaccagtggggaacactactgtagttagatctatttattgagcaccttgggcactgttgaagtgccttacattaatgaactcattcagtcaccatagcaacactctgagatgcagggactctgataacacccattttaaaggtgaggaaacaagcccagagaggttaagggaggagttcctgcccaccaggaacctgctttagtgggggatagtgaagaagacaataaaagatagtagttcaggcc

SEQ ID NO: 119 - protein, 390 aa, Homo sapiens
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 120 - RNA, 19 nt, synthetic construct
  misc_feature 1..19: note = synthetic construct
  source 1..19: mol_type = other RNA
tattgtcttcttcactatc

SEQ ID NO: 121 - RNA, 19 nt, synthetic construct
  misc_feature 1..19: note = synthetic construct
  source 1..19: mol_type = other RNA
tagatctaactacagtagt

SEQ ID NO: 122 - RNA, 19 nt, synthetic construct
  misc_feature 1..19: note = synthetic construct
  source 1..19: mol_type = other RNA
tatatgctgtgtgtactct

SEQ ID NO: 123 - RNA, 19 nt, synthetic construct
  misc_feature 1..19: note = synthetic construct
  source 1..19: mol_type = other RNA
tatatatgctgtgtgtact

SEQ ID NO: 124 - DNA, 31 bp, synthetic construct
  misc_feature 1..31: note = synthetic construct
  source 1..31: mol_type = other DNA
ccggtcatatatgctgtgtgtactcgaccgg

SEQ ID NO: 125 - DNA, 31 bp, synthetic construct
  misc_feature 1..31: note = synthetic construct
  source 1..31: mol_type = other DNA
ccggtcttttattgtcttcttcactgaccgg

SEQ ID NO: 126 - DNA, 31 bp, synthetic construct
  misc_feature 1..31: note = synthetic construct
  source 1..31: mol_type = other DNA
ccggtctatatatgctgtgtgtactgaccgg

SEQ ID NO: 127 - DNA, 5966 bp, Homo sapiens
  source 1..5966: mol_type = unassigned DNA
gtgatgttatctgctggcagcagaaggttcgctccgagcggagctccagaagctcctgacaagagaaagacagattgagatagagatagaaagagaaagagagaaagagacagcagagcgagagcgcaagtgaaagaggcaggggagggggatggagaatattagcctgacggtctagggagtcatccaggaacaaactgaggggctgcccggctgcagacaggaggagacagagaggatctattttagggtggcaagtgcctacctaccctaagcgagcaattccacgttggggagaagccagcagaggttgggaaagggtgggagtccaagggagcccctgcgcaaccccctcaggaataaaactccccagccagggtgtcgcaagggctgccgttgtgatccgcagggggtgaacgcaaccgcgacggctgatcgtctgtggctgggttggcgtttggagcaagagaaggaggagcaggagaaggagggagctggaggctggaagcgtttgcaagcggcggcggcagcaacgtggagtaaccaagcgggtcagcgcgcgcccgccagggtgtaggccacggagcgcagctcccagagcaggatccgcgccgcctcagcagcctctgcggcccctgcggcacccgaccgagtaccgagcgccctgcgaagcgcaccctcctccccgcggtgcgctgggctcgcccccagcgcgcgcacacgcacacacacacacacacacacacacgcacgcacacacgtgtgcgcttctctgctccggagctgctgctgctcctgctctcagcgccgcagtggaaggcaggaccgaaccgctccttctttaaatatataaatttcagcccaggtcagcctcggcggcccccctcaccgcgctcccggcgcccctcccgtcagttcgccagctgccagccccgggaccttttcatctcttcccttttggccggaggagccgagttcagatccgccactccgcacccgagactgacacactgaactccacttcctcctcttaaatttatttctacttaatagccactcgtctctttttttccccatctcattgctccaagaatttttttcttcttactcgccaaagtcagggttccctctgcccgtcccgtattaatatttccacttttggaactactggccttttctttttaaaggaattcaagcaggatacgtttttctgttgggcattgactagattgtttgcaaaagtttcgcatcaaaaacaacaacaacaaaaaaccaaacaactctccttgatctatactttgagaattgttgatttcttttttttattctgacttttaaaaacaacttttttttccacttttttaaaaaatgcactactgtgtgctgagcgcttttctgatcctgcatctggtcacggtcgcgctcagcctgtctacctgcagcacactcgatatggaccagttcatgcgcaagaggatcgaggcgatccgcgggcagatcctgagcaagctgaagctcaccagtcccccagaagactatcctgagcccgaggaagtccccccggaggtgatttccatctacaacagcaccagggacttgctccaggagaaggcgagccggagggcggccgcctgcgagcgcgagaggagcgacgaagagtactacgccaaggaggtttacaaaatagacatgccgcccttcttcccctccgaaactgtctgcccagttgttacaacaccctctggctcagtgggcagcttgtgctccagacagtcccaggtgctctgtgggtaccttgatgccatcccgcccactttctacagaccctacttcagaattgttcgatttgacgtctcagcaatggagaagaatgcttccaatttggtgaaagcagagttcagagtctttcgtttgcagaacccaaaagccagagtgcctgaacaacggattgagctatatcagattctcaagtccaaagatttaacatctccaacccagcgctacatcgacagcaaagttgtgaaaacaagagcagaaggcgaatggctctccttcgatgtaactgatgctgttcatgaatggcttcaccataaagacaggaacctgggatttaaaataagcttacactgtccctgctgcacttttgtaccatctaataattacatcatcccaaataaaagtgaagaactagaagcaagatttgcaggtattgatggcacctccacatataccagtggtgatcagaaaactataaagtccactaggaaaaaaaacagtgggaagaccccacatctcctgctaatgttattgccctcctacagacttgagtcacaacagaccaaccggcggaagaagcgtgctttggatgcggcctattgctttagaaatgtgcaggataattgctgcctacgtccactttacattgatttcaagagggatctagggtggaaatggatacacgaacccaaagggtacaatgccaacttctgtgctggagcatgcccgtatttatggagttcagacactcagcacagcagggtcctgagcttatataataccataaatccagaagcatctgcttctccttgctgcgtgtcccaagatttagaacctctaaccattctctactacattggcaaaacacccaagattgaacagctttctaatatgattgtaaagtcttgcaaatgcagctaaaattcttggaaaagtggcaagaccaaaatgacaatgatgatgataatgatgatgacgacgacaacgatgatgcttgtaacaagaaaacataagagagccttggttcatcagtgttaaaaaatttttgaaaaggcggtactagttcagacactttggaagtttgtgttctgtttgttaaaactggcatctgacacaaaaaaagttgaaggccttattctacatttcacctactttgtaagtgagagagacaagaagcaaattttttttaaagaaaaaaataaacactggaagaatttattagtgttaattatgtgaacaacgacaacaacaacaacaacaacaaacaggaaaatcccattaagtggagttgctgtacgtaccgttcctatcccgcgcctcacttgatttttctgtattgctatgcaataggcacccttcccattcttactcttagagttaacagtgagttatttattgtgtgttactatataatgaacgtttcattgcccttggaaaataaaacaggtgtataaagtggagaccaaatactttgccagaaactcatggatggcttaaggaacttgaactcaaacgagccagaaaaaaagaggtcatattaatgggatgaaaacccaagtgagttattatatgaccgagaaagtctgcattaagataaagaccctgaaaacacatgttatgtatcagctgcctaaggaagcttcttgtaaggtccaaaaactaaaaagactgttaataaaagaaactttcagtcagaataagtctgtaagtttttttttttctttttaattgtaaatggttctttgtcagtttagtaaaccagtgaaatgttgaaatgttttgacatgtactggtcaaacttcagaccttaaaatattgctgtatagctatgctataggttttttcctttgttttggtatatgtaaccatacctatattattaaaatagatggatatagaagccagcataattgaaaacacatctgcagatctcttttgcaaactattaaatcaaaacattaactactttatgtgtaatgtgtaaatttttaccatattttttatattctgtaataatgtcaactatgatttagattgacttaaatttgggctctttttaatgatcactcacaaatgtatgtttcttttagctggccagtacttttgagtaaagcccctatagtttgacttgcactacaaatgcattttttttttaataacatttgccctacttgtgctttgtgtttctttcattattatgacataagctacctgggtccacttgtcttttcttttttttgtttcacagaaaagatgggttcgagttcagtggtcttcatcttccaagcatcattactaaccaagtcagacgttaacaaatttttatgttaggaaaaggaggaatgttatagatacatagaaaattgaagtaaaatgttttcattttagcaaggatttagggttctaactaaaactcagaatctttattgagttaagaaaagtttctctaccttggtttaatcaatatttttgtaaaatcctattgttattacaaagaggacacttcataggaaacatctttttctttagtcaggtttttaatattcagggggaaattgaaagatatatattttagtcgatttttcaaaaggggaaaaaagtccaggtcagcataagtcattttgtgtatttcactgaagttataaggtttttataaatgttctttgaaggggaaaaggcacaagccaatttttcctatgatcaaaaaattctttctttcctctgagtgagagttatctatatctgaggctaaagtttaccttgctttaataaataatttgccacatcattgcagaagaggtatcctcatgctggggttaatagaatatgtcagtttatcacttgtcgcttatttagctttaaaataaaaattaataggcaaagcaatggaatatttgcagtttcacctaaagagcagcataaggaggcgggaatccaaagtgaagttgtttgatatggtctacttcttttttggaatttcctgaccattaattaaagaattggatttgcaagtttgaaaactggaaaagcaagagatgggatgccataatagtaaacagcccttgtgttggatgtaacccaatcccagatttgagtgtgtgttgattatttttttgtcttccacttttctattatgtgtaaatcacttttatttctgcagacattttcctctcagataggatgacattttgttttgtattattttgtctttcctcatgaatgcactgataatattttaaatgctctattttaagatctcttgaatctgttttttttttttttaatttgggggttctgtaaggtctttatttcccataagtaaatattgccatgggaggggggtggaggtggcaaggaaggggtgaagtgctagtatgcaagtgggcagcaattatttttgtgttaatcagcagtacaatttgatcgttggcatggttaaaaaatggaatataagattagctgttttgtattttgatgaccaattacgctgtattttaacacgatgtatgtctgtttttgtggtgctctagtggtaaataaattatttcgatgatatgtggatgtctttttcctatcagtaccatcatcgagtctagaaaacacctgtgatgcaataagactatctcaagctggaaaagtcataccacctttccgattgccctctgtgctttctcccttaaggacagtcacttcagaagtcatgctttaaagcacaagagtcaggccatatccatcaaggatagaagaaatccctgtgccgtctttttattcccttatttattgctatttggtaattgtttgagatttagtttccatccagcttgactgccgaccagaaaaaatgcagagagatgtttgcaccatgctttggctttctggttctatgttctgccaacgccagggccaaaagaactggtctagacagtatcccctgtagccccataacttggatagttgctgagccagccagatataacaagagccacgtgctttctggggttggttgtttgggatcagctacttgcctgtcagtttcactggtaccactgcaccacaaacaaaaaaacccaccctatttcctccaatttttttggctgctacctacaagaccagactcctcaaacgagttgccaatctcttaataaataggattaataaaaaaagtaattgtgactcaaaaaaaaaaaaaa

SEQ ID NO: 128 - protein, 442 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSETVCPVVTTPSGSVGSLCSRQSQVLCGYLDAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 129 - RNA, 20 nt, synthetic construct
  misc_feature 1..20: note = synthetic construct
  source 1..20: mol_type = other RNA
tatctctatctcaatctgtc

SEQ ID NO: 130 - RNA, 20 nt, synthetic construct
  misc_feature 1..20: note = synthetic construct
  source 1..20: mol_type = other RNA
ttctatctctatctcaatct

SEQ ID NO: 131 - RNA, 20 nt, synthetic construct
  misc_feature 1..20: note = synthetic construct
  source 1..20: mol_type = other RNA
ttctctttctatctctatct

SEQ ID NO: 132 - RNA, 20 nt, synthetic construct
  misc_feature 1..20: note = synthetic construct
  source 1..20: mol_type = other RNA
tctatctctatctcaatctg

SEQ ID NO: 133 - DNA, 31 bp, synthetic construct
  misc_feature 1..31: note = synthetic construct
  source 1..31: mol_type = other DNA
ccggtcttctatctctatctcaatcgaccgg

SEQ ID NO: 134 - DNA, 31 bp, synthetic construct
  misc_feature 1..31: note = synthetic construct
  source 1..31: mol_type = other DNA
ccggtctatctctatctcaatctgtgaccgg

SEQ ID NO: 135 - DNA, 31 bp, synthetic construct
  misc_feature 1..31: note = synthetic construct
  source 1..31: mol_type = other DNA
ccggtcttctctttctatctctatcgaccgg

SEQ ID NO: 136 - DNA, 7321 bp, Homo sapiens
  source 1..7321: mol_type = unassigned DNA
ttttgtagataaatgtgaggattttctctaaatccctcttctgtttgctaaatctcactgtcactgctaaattcagagcagatagagcctgcgcaatggaataaagtcctcaaaattgaaatgtgacattgctctcaacatctcccatctctctggatttctttttgcttcattattcctgctaaccaattcattttcagactttgtacttcagaagcaatgggaaaaatcagcagtcttccaacccaattatttaagtgctgcttttgtgatttcttgaaggtgaagatgcacaccatgtcctcctcgcatctcttctacctggcgctgtgcctgctcaccttcaccagctctgccacggctggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagctcgctctgtccgtgcccagcgccacaccgacatgcccaagacccagaaggaagtacatttgaagaacgcaagtagagggagtgcaggaaacaagaactacaggatgtaggaagaccctcctgaggagtgaagagtgacatgccaccgcaggatcctttgctctgcacgagttacctgttaaactttggaacacctaccaaaaaataagtttgataacatttaaaagatgggcgtttcccccaatgaaatacacaagtaaacattccaacattgtctttaggagtgatttgcaccttgcaaaaatggtcctggagttggtagattgctgttgatcttttatcaataatgttctatagaaaagaaaaaaaaaatatatatatatatatatcttagtccctgcctctcaagagccacaaatgcatgggtgttgtatagatccagttgcactaaattcctctctgaatcttggctgctggagccattcattcagcaaccttgtctaagtggtttatgaattgtttccttatttgcacttctttctacacaactcgggctgtttgttttacagtgtctgataatcttgttagtctatacccaccacctcccttcataacctttatatttgccgaatttggcctcctcaaaagcagcagcaagtcgtcaagaagcacaccaattctaacccacaagattccatctgtggcatttgtaccaaatataagttggatgcattttattttagacacaaagctttatttttccacatcatgcttacaaaaaagaataatgcaaatagttgcaactttgaggccaatcatttttaggcatatgttttaaacatagaaagtttcttcaactcaaaagagttccttcaaatgatgagttaatgtgcaacctaattagtaactttcctctttttattttttccatatagagcactatgtaaatttagcatatcaattatacaggatatatcaaacagtatgtaaaactctgttttttagtataatggtgctattttgtagtttgttatatgaaagagtctggccaaaacggtaatacgtgaaagcaaaacaataggggaagcctggagccaaagatgacacaaggggaagggtactgaaaacaccatccatttgggaaagaaggcaaagtccccccagttatgccttccaagaggaacttcagacacaaaagtccactgatgcaaattggactggcgagtccagagaggaaactgtggaatggaaaaagcagaaggctaggaattttagcagtcctggtttctttttctcatggaagaaatgaacatctgccagctgtgtcatggactcaccactgtgtgaccttgggcaagtcacttcacctctctgtgcctcagtttcctcatctgcaaaatgggggcaatatgtcatctacctacctcaaaggggtggtataaggtttaaaaagataaagattcagattttttttaccctgggttgctgtaagggtgcaacatcagggcgcttgagttgctgagatgcaaggaattctataaataacccattcatagcatagctagagattggtgaattgaatgctcctgacatctcagttcttgtcagtgaagctatccaaataactggccaactagttgttaaaagctaacagctcaatctcttaaaacacttttcaaaatatgtgggaagcatttgattttcaatttgattttgaattctgcatttggttttatgaatacaaagataagtgaaaagagagaaaggaaaagaaaaaggagaaaaacaaagagatttctaccagtgaaaggggaattaattactctttgttagcactcactgactcttctatgcagttactacatatctagtaaaacctcgtttaatactataaataatattctattcattttgaaaaacacaatgattccttcttttctaggcaatataaggaaagtgatccaaaatttgaaatattaaaataatatctaataaaaagtcacaaagttatcttctttaacaaactttactcttattcttagctgtatatacatttttttaaaagtttgttaaaatatgcttgactagagtttccagttgaaaggcaaaaacttccatcacaacaagaaatttcccatgcctgctcagaagggtagcccctagctctctgtgaatgtgttttatccattcaactgaaaattggtatcaagaaagtccactggttagtgtactagtccatcatagcctagaaaatgatccctatctgcagatcaagattttctcattagaacaatgaattatccagcattcagatctttctagtcaccttagaactttttggttaaaagtacccaggcttgattatttcatgcaaattctatattttacattcttggaaagtctatatgaaaaacaaaaataacatcttcagtttttctcccactgggtcacctcaaggatcagaggccaggaaaaaaaaaaaaaagactccctggatctctgaatatatgcaaaaagaaggccccatttagtggagccagcaatcctgttcagtcaacaagtattttaactctcagtccaacattatttgaattgagcacctcaagcatgcttagcaatgttctaatcactatggacagatgtaaaagaaactatacatcatttttgccctctgcctgttttccagacatacaggttctgtggaataagatactggactcctcttcccaagatggcacttctttttatttcttgtccccagtgtgtaccttttaaaattattccctctcaacaaaactttataggcagtcttctgcagacttaacgtgttttctgtcatagttagatgtgataattctaagagtgtctatgacttatttccttcacttaattctatccacagtcaaaaatcccccaaggaggaaagctgaaagatgcactgccatattatctttcttaactttttccaacacataatcctctccaactggattataaataaattgaaaataactcattataccaattcactattttattttttaatgaattaaaactagaaaacaaattgatgcaaaccctggaagtcagttgattactatatactacagcagaatgactcagatttcatagaaaggagcaaccaaaatgtcacaacccaaaactttacaagctttgcttcagaattagattgctttataattcttgaatgaggcaatttcaagatatttgtaaaagaacagtaaacattggtaagaatgagctttcaactcataggcttatttccaatttaattgaccatactggatacttaggtcaaatttctgttctctcttccccaaataatattaaagtattatttgaactttttaagatgaggcagttcccctgaaaaagttaatgcagctctccatcagaatccactcttctagggatatgaaaatctcttaacacccaccctacatacacagacacacacacacacacacacacacacacacacacacacattcaccctaaggatccaatggaatactgaaaagaaatcacttccttgaaaattttattaaaaaacaaacaaacaaacaaaaagcctgtccacccttgagaatccttcctctccttggaacgtcaatgtttgtgtagatgaaaccatctcatgctctgtggctccagggtttctgttactattttatgcacttgggagaaggcttagaataaaagatgtagcacattttgctttcccatttattgtttggccagctatgccaatgtggtgctattgtttctttaagaaagtacttgactaaaaaaaaaagaaaaaaagaaaaaaaagaaagcatagacatatttttttaaagtataaaaacaacaattctatagatagatggcttaataaaatagcattaggtctatctagccaccaccacctttcaactttttatcactcacaagtagtgtactgttcaccaaattgtgaatttgggggtgcaggggcaggagttggaaattttttaaagttagaaggctccattgttttgttggctctcaaacttagcaaaattagcaatatattatccaatcttctgaacttgatcaagagcatggagaataaacgcgggaaaaaagatcttataggcaaatagaagaatttaaaagataagtaagttccttattgatttttgtgcactctgctctaaaacagatattcagcaagtggagaaaataagaacaaagagaaaaaatacatagatttacctgcaaaaaatagcttctgccaaatcccccttgggtattctttggcatttactggtttatagaagacattctcccttcacccagacatctcaaagagcagtagctctcatgaaaagcaatcactgatctcatttgggaaatgttggaaagtatttccttatgagatgggggttatctactgataaagaaagaatttatgagaaattgttgaaagagatggctaacaatctgtgaagattttttgtttcttgtttttgttttttttttttttttactttatacagtctttatgaatttcttaatgttcaaaatgacttggttcttttcttctttttttatatcagaatgaggaataataagttaaacccacatagactctttaaaactataggctagatagaaatgtatgtttgacttgttgaagctataatcagactatttaaaatgttttgctatttttaatcttaaaagattgtgctaatttattagagcagaacctgtttggctctcctcagaagaaagaatctttccattcaaatcacatggctttccaccaatattttcaaaagataaatctgatttatgcaatggcatcatttattttaaaacagaagaattgtgaaagtttatgcccctcccttgcaaagaccataaagtccagatctggtaggggggcaacaacaaaaggaaaatgttgttgattcttggttttggattttgttttgttttcaatgctagtgtttaatcctgtagtacatatttgcttattgctattttaatattttataagaccttcctgttaggtattagaaagtgatacatagatatcttttttgtgtaatttctatttaaaaaagagagaagactgtcagaagctttaagtgcatatggtacaggataaagatatcaatttaaataaccaattcctatctggaacaatgcttttgttttttaaagaaacctctcacagataagacagaggcccaggggatttttgaagctgtctttattctgcccccatcccaacccagcccttattattttagtatctgcctcagaattttatagagggctgaccaagctgaaactctagaattaaaggaacctcactgaaaacatatatttcacgtgttccctctttttttttttcctttttgtgagatggggtctcgcactgtcccccaggctggagtgcagtggcatgatctcggctcactgcaacctccacctcctgggtttaagcgattctcctgcctcagcctcctgagtagctgggattacaggcacccaccactatgcccggctaattttttggatttttaatagagacggggttttaccatgttggccaggttggtctcaaactcctgaccttgtgatttgcccgcctcagcctcccaaattgctgggattacaggcatgagccaccacaccctgcccatgtgttccctcttaatgtatgattacatggatcttaaacatgatccttctctcctcattcttcaactatctttgatggggtctttcaaggggaaaaaaatccaagcttttttaaagtaaaaaaaaaaaaagagaggacacaaaaccaaatgttactgctcaactgaaatatgagttaagatggagacagagtttctcctaataaccggagctgaattacctttcactttcaaaaacatgaccttccacaatccttagaatctgcctttttttatattactgaggcctaaaagtaaacattactcattttattttgcccaaaatgcactgatgtaaagtaggaaaaataaaaacagagctctaaaatccctttcaagccacccattgaccccactcaccaactcatagcaaagtcacttctgttaatcccttaatctgattttgtttggatatttatcttgtacccgctgctaaacacactgcaggagggactctgaaacctcaagctgtctacttacatcttttatctgtgtctgtgtatcatgaaaatgtctattcaaaatatcaaaacctttcaaatatcacgcagcttatattcagtttacataaaggccccaaataccatgtcagatctttttggtaaaagagttaatgaactatgagaattgggattacatcatgtattttgcctcatgtatttttatcacacttataggccaagtgtgataaataaacttacagacactgaattaatttcccctgctactttgaaaccagaaaataatgactggccattcgttacatctgtcttagttgaaaagcatattttttattaaattaattctgattgtatttgaaattattattcaattcacttatggcagaggaatatcaatcctaatgacttctaaaaatgtaactaattgaatcattatcttacatttactgtttaataagcatattttgaaaatgtatggctagagtgtcataataaaatggtatatctttctttagtaattacattaaaattagtcatgtttgattaattagttc

SEQ ID NO: 137 - protein, 153 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM

SEQ ID NO: 138 - RNA, 19 nt, synthetic construct
  misc_feature 1..19: note = synthetic construct
  source 1..19: mol_type = other RNA
taaactgaatataagctgc

SEQ ID NO: 139 - RNA, 19 nt, synthetic construct
  misc_feature 1..19: note = synthetic construct
  source 1..19: mol_type = other RNA
taaaaaaatatgtctatgc

SEQ ID NO: 140 - RNA, 19 nt, synthetic construct
  misc_feature 1..19: note = synthetic construct
  source 1..19: mol_type = other RNA
tttaacaggtaactcgtgc

SEQ ID NO: 141 - RNA, 19 nt, synthetic construct
  misc_feature 1..19: note = synthetic construct
  source 1..19: mol_type = other RNA
taacaaactacaaaatagc

SEQ ID NO: 142 - DNA, 32 bp, synthetic construct
  misc_feature 1..32: note = synthetic construct
  source 1..32: mol_type = other DNA
ccggtctaaactgaatataagctgcggaccgg

SEQ ID NO: 143 - DNA, 32 bp, synthetic construct
  misc_feature 1..32: note = synthetic construct
  source 1..32: mol_type = other DNA
ccggtctttaaattcttctatttgccgaccgg

SEQ ID NO: 144 - DNA, 32 bp, synthetic construct
  misc_feature 1..32: note = synthetic construct
  source 1..32: mol_type = other DNA
ccggtctaatcaactgacttccaggggaccgg

SEQ ID NO: 145 - DNA, 3150 bp, Homo sapiens
  source 1..3150: mol_type = unassigned DNA
ccacaaagggcacttggccccagggctaggagagcgaggggagagcacagccacccgcctcggcggcccgggactcggctcgactcgccggagaatgcgcccgaggacgacggggcgccagagccgcggtgctttcaactggcgagcgcgaatgggggtgcactggagtaaggcagagtgatgcgggggggcaactcgcctggcaccgagatcgccgccgtgcccttccctggacccggcgtcgcccaggatggctgccccgagccatgggccgcggcggagctagcgcggagcgcccgaccctcgacccccgagtcccggagccggccccgcgcggggccacgcgtccctcgggcgctggttcctaaggaggacgacagcaccagcttctcctttctcccttcccttccctgccccgcactcctccccctgctcgctgttgttgtgtgtcagcacttggctggggacttcttgaacttgcagggagaataacttgcgcaccccactttgcgccggtgcctttgccccagcggagcctgcttcgccatctccgagccccaccgcccctccactcctcggccttgcccgacactgagacgctgttcccagcgtgaaaagagagactgcgcggccggcacccgggagaaggaggaggcaaagaaaaggaacggacattcggtccttgcgccaggtcctttgaccagagtttttccatgtggacgctctttcaatggacgtgtccccgcgtgcttcttagacggactgcggtctcctaaaggtcgaccatggtggccgggacccgctgtcttctagcgttgctgcttccccaggtcctcctgggcggcgcggctggcctcgttccggagctgggccgcaggaagttcgcggcggcgtcgtcgggccgcccctcatcccagccctctgacgaggtcctgagcgagttcgagttgcggctgctcagcatgttcggcctgaaacagagacccacccccagcagggacgccgtggtgcccccctacatgctagacctgtatcgcaggcactcaggtcagccgggctcacccgccccagaccaccggttggagagggcagccagccgagccaacactgtgcgcagcttccaccatgaagaatctttggaagaactaccagaaacgagtgggaaaacaacccggagattcttctttaatttaagttctatccccacggaggagtttatcacctcagcagagcttcaggttttccgagaacagatgcaagatgctttaggaaacaatagcagtttccatcaccgaattaatatttatgaaatcataaaacctgcaacagccaactcgaaattccccgtgaccagacttttggacaccaggttggtgaatcagaatgcaagcaggtgggaaagttttgatgtcacccccgctgtgatgcggtggactgcacagggacacgccaaccatggattcgtggtggaagtggcccacttggaggagaaacaaggtgtctccaagagacatgttaggataagcaggtctttgcaccaagatgaacacagctggtcacagataaggccattgctagtaacttttggccatgatggaaaagggcatcctctccacaaaagagaaaaacgtcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaactctgttaactctaagattcctaaggcatgctgtgtcccgacagaactcagtgctatctcgatgctgtaccttgacgagaatgaaaaggttgtattaaagaactatcaggacatggttgtggagggttgtgggtgtcgctagtacagcaaaattaaatacataaatatatatatatatatatattttagaaaaaagaaaaaaacaaacaaacaaaaaaaccccaccccagttgacactttaatatttcccaatgaagactttatttatggaatggaatggaaaaaaaaacagctattttgaaaatatatttatatctacgaaaagaagttgggaaaacaaatattttaatcagagaattattccttaaagatttaaaatgtatttagttgtacattttatatgggttcaaccccagcacatgaagtataatggtcagatttattttgtatttatttactattataaccactttttaggaaaaaaatagctaatttgtatttatatgtaatcaaaagaagtatcgggtttgtacataattttccaaaaattgtagttgttttcagttgtgtgtatttaagatgaaaagtctacatggaaggttactctggcaaagtgcttagcacgtttgcttttttgcagtgctactgttgagttcacaagttcaagtccagaaaaaaaaagtggataatccactctgctgactttcaagattattatattattcaattctcaggaatgttgcagagtgattgtccaatccatgagaatttacatccttattaggtggaatatttggataagaaccagacattgctgatctattatagaaactctcctcctgccccttaatttacagaaagaataaagcaggatccatagaaataattaggaaaacgatgaacctgcaggaaagtgaatgatggtttgttgttcttctttcctaaattagtgatcccttcaaaggggctgatctggccaaagtattcaataaaacgtaagatttcttcattattgatattgtggtcatatatatttaaaattgatatctcgtggccctcatcaagggttggaaatttatttgtgttttacctttacctcatctgagagctctttattctccaaagaacccagttttctaactttttgcccaacacgcagcaaaattatgcacatcgtgttttctgcccaccctctgttctctgacctatcagcttgcttttctttccaaggttgtgtgtttgaacacatttctccaaatgttaaacctatttcagataataaatatcaaatctctggcatttcattctataaagtc

SEQ ID NO: 146 - protein, 396 aa, Homo sapiens
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 147 - RNA, 19 nt, synthetic construct
  misc_feature 1..19: note = synthetic construct
  source 1..19: mol_type = other RNA
ttgtgaactcaacagtagc

SEQ ID NO: 148 - RNA, 19 nt, synthetic construct
  misc_feature 1..19: note = synthetic construct
  source 1..19: mol_type = other RNA
ttaattttgctgtactagc

SEQ ID NO: 149 - RNA, 19 nt, synthetic construct
  misc_feature 1..19: note = synthetic construct
  source 1..19: mol_type = other RNA
taaaacacaaataaatttc

SEQ ID NO: 150 - RNA, 19 nt, synthetic construct
  misc_feature 1..19: note = synthetic construct
  source 1..19: mol_type = other RNA
ttctttctgtaaattaagg

SEQ ID NO: 151 - DNA, 30 bp, synthetic construct
  misc_feature 1..30: note = synthetic construct
  source 1..30: mol_type = other DNA
ccggtctaatacaaaataaatctggaccgg

SEQ ID NO: 152 - DNA, 31 bp, synthetic construct
  misc_feature 1..31: note = synthetic construct
  source 1..31: mol_type = other DNA
ccggtcaaaacacaaataaatttccgaccgg

SEQ ID NO: 153 - DNA, 31 bp, synthetic construct
  misc_feature 1..31: note = synthetic construct
  source 1..31: mol_type = other DNA
ccggtcttcattctcgtcaaggtacgaccgg

SEQ ID NO: 154 - DNA, 1957 bp, Homo sapiens
  source 1..1957: mol_type = unassigned DNA
aagaggaggaaggaagatgcgagaaggcagaggaggagggagggagggaaggagcgcggagcccggcccggaagctaggtgagtgtggcatccgagctgagggacgcgagcctgagacgccgctgctgctccggctgagtatctagcttgtctccccgatgggattcccgtccaagctatctcgagcctgcagcgccacagtccccggccctcgcccaggttcactgcaaccgttcagaggtccccaggagctgctgctggcgagcccgctactgcagggacctatggagccattccgtagtgccatcccgagcaacgcactgctgcagcttccctgagcctttccagcaagtttgttcaagattggctgtcaagaatcatggactgttattatatgccttgttttctgtcaagacaccatgattcctggtaaccgaatgctgatggtcgttttattatgccaagtcctgctaggaggcgcgagccatgctagtttgatacctgagacggggaagaaaaaagtcgccgagattcagggccacgcgggaggacgccgctcagggcagagccatgagctcctgcgggacttcgaggcgacacttctgcagatgtttgggctgcgccgccgcccgcagcctagcaagagtgccgtcattccggactacatgcgggatctttaccggcttcagtctggggaggaggaggaagagcagatccacagcactggtcttgagtatcctgagcgcccggccagccgggccaacaccgtgaggagcttccaccacgaagaacatctggagaacatcccagggaccagtgaaaactctgcttttcgtttcctctttaacctcagcagcatccctgagaacgaggtgatctcctctgcagagcttcggctcttccgggagcaggtggaccagggccctgattgggaaaggggcttccaccgtataaacatttatgaggttatgaagcccccagcagaagtggtgcctgggcacctcatcacacgactactggacacgagactggtccaccacaatgtgacacggtgggaaacttttgatgtgagccctgcggtccttcgctggacccgggagaagcagccaaactatgggctagccattgaggtgactcacctccatcagactcggacccaccagggccagcatgtcaggattagccgatcgttacctcaagggagtgggaattgggcccagctccggcccctcctggtcacctttggccatgatggccggggccatgccttgacccgacgccggagggccaagcgtagccctaagcatcactcacagcgggccaggaagaagaataagaactgccggcgccactcgctctatgtggacttcagcgatgtgggctggaatgactggattgtggccccaccaggctaccaggccttctactgccatggggactgcccctttccactggctgaccacctcaactcaaccaaccatgccattgtgcagaccctggtcaattctgtcaattccagtatccccaaagcctgttgtgtgcccactgaactgagtgccatctccatgctgtacctggatgagtatgataaggtggtactgaaaaattatcaggagatggtagtagagggatgtgggtgccgctgagatcaggcagtccttgaggatagacagatatacacaccacacacacacaccacatacaccacacacacacgttcccatccactcacccacacactacacagactgcttccttatagctggacttttatttaaaaaaaaaaaaaaaaaaggaaaaaatccctaaacattcaccttgaccttatttatgactttacgtgcaaatgttttgaccatattgatcatatattttgacaaaatatatttataactacgtattaaaagaaaaaaataaaatgagtcattattttaaaggtaaaaaaaaaaaaaaaaaa

SEQ ID NO: 155 - protein, 408 aa, Homo sapiens
MIPGNRMLMVVLLCQVLLGGASHASLIPETGKKKVAEIQGHAGGRRSGQSHELLRDFEATLLQMFGLRRRPQPSKSAVIPDYMRDLYRLQSGEEEEEQIHSTGLEYPERPASRANTVRSFHHEEHLENIPGTSENSAFRFLFNLSSIPENEVISSAELRLFREQVDQGPDWERGFHRINIYEVMKPPAEVVPGHLITRLLDTRLVHHNVTRWETFDVSPAVLRWTREKQPNYGLAIEVTHLHQTRTHQGQHVRISRSLPQGSGNWAQLRPLLVTFGHDGRGHALTRRRRAKRSPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 156 - RNA, 20 nt, synthetic construct
  misc_feature 1..20: note = synthetic construct
  source 1..20: mol_type = other RNA
taataaaacgaccatcagca

SEQ ID NO: 157 - RNA, 20 nt, synthetic construct
  misc_feature 1..20: note = synthetic construct
  source 1..20: mol_type = other RNA
tatctgtctatcctcaagga

SEQ ID NO: 158 - RNA, 20 nt, synthetic construct
  misc_feature 1..20: note = synthetic construct
  source 1..20: mol_type = other RNA
ttcttattcttcttcctggc

SEQ ID NO: 159 - RNA, 19 nt, synthetic construct
  misc_feature 1..19: note = synthetic construct
  source 1..19: mol_type = other RNA
taataaaacgaccatcagc

SEQ ID NO: 160 - DNA, 31 bp, synthetic construct
  misc_feature 1..31: note = synthetic construct
  source 1..31: mol_type = other DNA
ccggtctatctgtctatcctcaagggaccgg

SEQ ID NO: 161 - DNA, 31 bp, synthetic construct
  misc_feature 1..31: note = synthetic construct
  source 1..31: mol_type = other DNA
ccggtctctcaggtatcaaactagcgaccgg

SEQ ID NO: 162 - DNA, 31 bp, synthetic construct
  misc_feature 1..31: note = synthetic construct
  source 1..31: mol_type = other DNA
ccggtctttgtcaaaatatatgatcgaccgg

SEQ ID NO: 163 - DNA, 4049 bp, Homo sapiens
  source 1..4049: mol_type = unassigned DNA
agcgcgtaccactctggcgctcccgaggcggcctcttgtgcgatccagggcgcacaaggctgggagagcgccccggggcccctgctatccgcgccggaggttggaagagggtgggttgccgccgcccgagggcgagagcgccagaggagcgggaagaaggagcgctcgcccgcccgcctgcctcctcgctgcctccccggcgttggctctctggactcctaggcttgctggctgctcctcccacccgcgcccgcctcctcactcgccttttcgttcgccggggctgctttccaagccctgcggtgcgcccgggcgagtgcggggcgaggggcccggggccagcaccgagcagggggcgggggtccgggcagagcgcggccggccggggaggggccatgtctggcgcgggcgcagcggggcccgtctgcagcaagtgaccgagcggcgcggacggccgcctgccccctctgccacctggggcggtgcgggcccggagcccggagcccgggtagcgcgtagagccggcgcgatgcacgtgcgctcactgcgagctgcggcgccgcacagcttcgtggcgctctgggcacccctgttcctgctgcgctccgccctggccgacttcagcctggacaacgaggtgcactcgagcttcatccaccggcgcctccgcagccaggagcggcgggagatgcagcgcgagatcctctccattttgggcttgccccaccgcccgcgcccgcacctccagggcaagcacaactcggcacccatgttcatgctggacctgtacaacgccatggcggtggaggagggcggcgggcccggcggccagggcttctcctacccctacaaggccgtcttcagtacccagggcccccctctggccagcctgcaagatagccatttcctcaccgacgccgacatggtcatgagcttcgtcaacctcgtggaacatgacaaggaattcttccacccacgctaccaccatcgagagttccggtttgatctttccaagatcccagaaggggaagctgtcacggcagccgaattccggatctacaaggactacatccgggaacgcttcgacaatgagacgttccggatcagcgtttatcaggtgctccaggagcacttgggcagggaatcggatctcttcctgctcgacagccgtaccctctgggcctcggaggagggctggctggtgtttgacatcacagccaccagcaaccactgggtggtcaatccgcggcacaacctgggcctgcagctctcggtggagacgctggatgggcagagcatcaaccccaagttggcgggcctgattgggcggcacgggccccagaacaagcagcccttcatggtggctttcttcaaggccacggaggtccacttccgcagcatccggtccacggggagcaaacagcgcagccagaaccgctccaagacgcccaagaaccaggaagccctgcggatggccaacgtggcagagaacagcagcagcgaccagaggcaggcctgtaagaagcacgagctgtatgtcagcttccgagacctgggctggcaggactggatcatcgcgcctgaaggctacgccgcctactactgtgagggggagtgtgccttccctctgaactcctacatgaacgccaccaaccacgccatcgtgcagacgctggtccacttcatcaacccggaaacggtgcccaagccctgctgtgcgcccacgcagctcaatgccatctccgtcctctacttcgatgacagctccaacgtcatcctgaagaaatacagaaacatggtggtccgggcctgtggctgccactagctcctccgagaattcagaccctttggggccaagtttttctggatcctccattgctcgccttggccaggaaccagcagaccaactgccttttgtgagaccttcccctccctatccccaactttaaaggtgtgagagtattaggaaacatgagcagcatatggcttttgatcagtttttcagtggcagcatccaatgaacaagatcctacaagctgtgcaggcaaaacctagcaggaaaaaaaaacaacgcataaagaaaaatggccgggccaggtcattggctgggaagtctcagccatgcacggactcgtttccagaggtaattatgagcgcctaccagccaggccacccagccgtgggaggaagggggcgtggcaaggggtgggcacattggtgtctgtgcgaaaggaaaattgacccggaagttcctgtaataaatgtcacaataaaacgaatgaatgaaaatggttaggacgttacagatatattttcctaaacaatttatccccatttctcggtttatcctgatgcgtaaacagaagctgtgtcaagtggagggcggggaggtccctctccattccctacagttttcatcctgaggcttgcagaggcccagtgtttaccgaggtttgcccaaatccaagatctagtgggaggggaaagagcaaatgtctgctccgaggagggcggtgtgttgatctttggaggaaaaatatgttctgttgttcagctggatttgccgtggcagaaatgaaactaggtgtgtgaaatacccgcagacatttgggattggcttttcacctcgccccagtggtagtaaatccatgtgaaattgcagaggggacaaggacagcaagtaggatggaacttgcaactcaaccctgttgttaagaagcaccaatgggccgggcacagtagctcccacctgtaatcccagcactttgggaggctgaggtgggcggatcatttgaggtcaggagttcgagaccagcctggccaacatggtgaaaccccatctctactaaaaatacaaaaattagccgggcatggtggcacgcacctgtaatcccagctactctggaggctgaggcaggagaattgcttgaaccccagaggtggaggttgcagtgagccaagatcgtcccactgcactccagcttgggtgacaaaacaagactccatctcaaaagaaaaaaaaaacagcaccaatgaagcctagttctccacgggagtggggtgagcaggagcactgcacatcgccccagtggaccctctggtctttgtctgcagtggcattccaaggctgggccctggcaagggcacccgtggctgtctcttcatttgcagaccctgatcagaagtctctgcaaacaaatttgctccttgaattaagggggagatggcataataggaggtctgatgggtgcaggatgtgctggacttacattgcaaatagaagccttgttgagggtgacatcctaaccaagtgtcccgatttggaggtggcatttctgacgtggctcttggtgtaagcctgccttgccttggctggtgagtcccataaatagtatgcactcagcctccggccacaaacacaaggcctaggggagggctagactgtctgcaaacgttttctgcatctgtaaagaaaacaaggtgatcgaaaactgtggccatgtggaacccggtcttgtgggggactgtttctccatcttgactcagacagttcctggaaacaccggggctctgtttttattttctttgatgtttttcttctttagtagcttgggctgcagcctccactctctagtcactggggaggagtattttttgttatgtttggtttcatttgctggcagagctggggctttttgtgtgatccctcttggtgtgagttttctgacccaaccagcctctggttagcatcatttgtacatttaaacctgtaaatagttgttacaaagcaaagagattatttatttccatccaaagctcttttgaacacccccccccctttaatccctcgttcaggacgatgagcttgctttccttcaacctgtttgttttcttatttaagactatttattaatggttggaccaatgtactcacagctgttgcgtcgagcagtccttagtgaaaattctgtataaatagacaaaatgaaaagggtttgaccttgcaataaaaggagacgtttggttctggcaaaaaaaaaaaaaaaaaa

SEQ ID NO: 164 - protein, 431 aa, Homo sapiens
MHVRSLRAAAPHSFVALWAPLFLLRSALADFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETFRISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 165 - RNA, 19 nt, synthetic construct
  misc_feature 1..19: note = synthetic construct
  source 1..19: mol_type = other RNA
ttcctaatactctcacacc

SEQ ID NO: 166 - RNA, 19 nt, synthetic construct
  misc_feature 1..19: note = synthetic construct
  source 1..19: mol_type = other RNA
taacaaaaaatactcctcc

SEQ ID NO: 167 - RNA, 20 nt, synthetic construct
  misc_feature 1..20: note = synthetic construct
  source 1..20: mol_type = other RNA
taaataagaaaacaaacagg

SEQ ID NO: 168 - RNA, 20 nt, synthetic construct
  misc_feature 1..20: note = synthetic construct
  source 1..20: mol_type = other RNA
ttcctaatactctcacacct

SEQ ID NO: 169 - DNA, 32 bp, synthetic construct
  misc_feature 1..32: note = synthetic construct
  source 1..32: mol_type = other DNA
ccggtctaacaaaaaatactcctcccgaccgg

SEQ ID NO: 170 - DNA, 32 bp, synthetic construct
  misc_feature 1..32: note = synthetic construct
  source 1..32: mol_type = other DNA
  modified_base 17: mod_base = OTHER; note = Uracil
ccggtcttgtaacaactatttacagggaccgg

SEQ ID NO: 171 - DNA, 31 bp, synthetic construct
  misc_feature 1..31: note = synthetic construct
  source 1..31: mol_type = other DNA
ccggtctaaataagaaaacaaacaggaccgg

SEQ ID NO: 172 - DNA, 1802 bp, Homo sapiens
  source 1..1802: mol_type = unassigned DNA
gggcagctccaccctgggagggactgtggcccaggtactgcccgggtgctactttatgggcagcagctcagttgagttagagtctggaagacctcagaagacctcctgtcctatgaggccctccccatggctttagagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagtactgcccaggcctgcctgttcccattcttgcatggcaaggactgcagggactgccagtccccctgccccagggctcccggctatgggggcactgaggaccagccattgaggggtggaccctcagaaggcgtcacaacaacctggtcacaggactctgcctcctcttcaactgaccagcctccatgctgcctccagaatggtctttctaatgtgtgaatcagagcacagcagcccctgcacaaagcccttccatgtcgcctctgcattcaggatcaaaccccgaccacctgcccaacctgctctcctcttgccactgcctcttcctccctcattccaccttcccatgccctggatccatcaggccacttgatgacccccaaccaagtggctcccacaccctgttttacaaaaaagaaaagaccagtccatgagggaggtttttaagggtttgtggaaaatgaaaattaggatttcatgatttttttttttcagtccccgtgaaggagagcccttcatttggagattatgttctttcggggagaggctgaggacttaaaatattcctgcatttgtgaaatgatggtgaaagtaagtggtagcttttcccttctttttcttctttttttgtgatgtcccaacttgtaaaaattaaaagttatggtactatgttagccccataattttttttttccttttaaaacacttccataatctggactcctctgtccaggcactgctgcccagcctccaagctccatctccactccagattttttacagctgcctgcagtactttacctcctatcagaagtttctcagctcccaaggctctgagcaaatgtggctcctgggggttctttcttcctctgctgaaggaataaattgctccttgacattgtagagcttctggcacttggagacttgtatgaaagatggctgtgcctctgcctgtctcccccaccgggctgggagctctgcagagcaggaaacatgactcgtatatgtctcaggtccctgcagggccaagcacctagcctcgctcttggcaggtactcagcgaatgaatgctgtatatgttgggtgcaaagttccctacttcctgtgacttcagctctgttttacaataaaatcttgaaaatgcctaaaaaaaaaaaaaaaaaaaaaaaaaaaaaaaaaaaaaaaaaaaaaaaaaaaa

SEQ ID NO: 173 - protein, 159 aa, Homo sapiens
MALETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 174 - RNA, 100 nt, synthetic construct
  misc_feature 1..100: note = synthetic construct
  source 1..100: mol_type = other RNA
tgtgtctctctctgtgtcctgccagtggttttaccctatggtaggttacgtcatgctgttctaccacagggtagaaccacggacaggataccggggcacc

SEQ ID NO: 175 - RNA, 22 nt, synthetic construct
  misc_feature 1..22: note = synthetic construct
  source 1..22: mol_type = other RNA
cagtggttttaccctatggtag

SEQ ID NO: 176 - RNA, 87 nt, Homo sapiens
  source 1..87: mol_type = unassigned RNA
accgcagggaaaatgagggacttttgggggcagatgtgtttccattccactatcataatgcccctaaaaatccttattgctcttgca

SEQ ID NO: 177 - RNA, 23 nt, synthetic construct
  misc_feature 1..23: note = synthetic construct
  source 1..23: mol_type = other RNA
agggacttttgggggcagatgtg

SEQ ID NO: 178 - RNA, 86 nt, Homo sapiens
  source 1..86: mol_type = unassigned RNA
tgccagtctctaggtccctgagaccctttaacctgtgaggacatccagggtcacaggtgaggttcttgggagcctggcgtctggcc

SEQ ID NO: 179 - RNA, 24 nt, synthetic construct
  misc_feature 1..24: note = synthetic construct
  source 1..24: mol_type = other RNA
tccctgagaccctttaacctgtga

SEQ ID NO: 180 - RNA, 22 nt, synthetic construct
  misc_feature 1..22: note = synthetic construct
  source 1..22: mol_type = other RNA
acaggtgaggttcttgggagcc

SEQ ID NO: 181 - DNA, 1968 bp, Homo sapiens
  source 1..1968: mol_type = unassigned DNA
actccgggtggcaggcgcccgggggaatcccagctgactcgctcactgccttcgaagtccggcgccccccgggagggaactgggtggccgcaccctcccggctgcggtggctgtcgccccccaccctgcagccaggactcgatggaggtacagagctcggcttctttgccttgggaggggagtggtggtggttgaaagggcgatggaattttccccgaaagcctacgcccagggcccctcccagctccagcgttaccctccggtctatcctactggccgagctgccccgccttctcatggggaaaacttagccgcaacttcaatttttggtttttcctttaatgacacttctgaggctctcctagccatcctcccgcttccggaggagcgcagatcgcaggtccctttgcccctggcgtgcgactccctactgcgctgcgctcttacggcgttccaggctgctggctagcgcaaggcgggccgggcaccccgcgctccgctgggagggtgagggacgcgcgtctggcggccccagccaagctgcgggtttctgagaagacgctgtcccgcagccctgagggctgagttctgcacccagtcaagctcaggaaggccaagaaaagaatccattccaatatatggccatgtggctctttggagcaatgttccatcatgttccatgctgctgacgtcacatggagcacagaaatcaatgttagcagatagccagcccatacaagatcgtattgtattgtaggaggcatcgtggatggatggctgctggaaaccccttgccatagccagctcttcttcaatacttaaggatttaccgtggctttgagtaatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgattgcaattgattctttttaaagtgtttctgttattaacaaacatcactctgctgcttagacataacaaaacactcggcatttcaaatgtgctgtcaaaacaagtttttctgtcaagaagatgatcagaccttggatcagatgaactcttagaaatgaaggcagaaaaatgtcattgagtaatatagtgactatgaacttctctcagacttactttactcatttttttaatttattattgaaattgtacatatttgtggaataatgtaaaatgttgaataaaaatatgtacaagtgttgttttttaagttgcactgatattttacctcttattgcaaaatagcatttgtttaagggtgatagtcaaattatgtattggtggggctgggtaccaatgctgcaggtcaacagctatgctggtaggctcctgcctgtgtggaaccactgactactggctctcattgacttccttactaagcatagcaaacagaggaagaatttgttatcagtaagaaaaagaagaactatatgtgaatcctcttctttacactgtaatttagttattgatgtataaagcaactgttatgaaataaagaaattgcaataactggcaaaaaaaaaaaaaaaaaaaaaaaaa

SEQ ID NO: 182 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 183 - DNA, 1252 bp, Homo sapiens
  source 1..1252: mol_type = unassigned DNA
ctttgaattcctagctcctgtggtctccagatttcaggcctaagatgaaagcctctagtcttgccttcagccttctctctgctgcgttttatctcctatggactccttccactggactgaagacactcaatttgggaagctgtgtgatcgccacaaaccttcaggaaatacgaaatggattttctgagatacggggcagtgtgcaagccaaagatggaaacattgacatcagaatcttaaggaggactgagtctttgcaagacacaaagcctgcgaatcgatgctgcctcctgcgccatttgctaagactctatctggacagggtatttaaaaactaccagacccctgaccattatactctccggaagatcagcagcctcgccaattcctttcttaccatcaagaaggacctccggctctgtcatgcccacatgacatgccattgtggggaggaagcaatgaagaaatacagccagattctgagtcactttgaaaagctggaacctcaggcagcagttgtgaaggctttgggggaactagacattcttctgcaatggatggaggagacagaataggaggaaagtgatgctgctgctaagaatattcgaggtcaagagctccagtcttcaatacctgcagaggaggcatgaccccaaaccaccatctctttactgtactagtcttgtgctggtcacagtgtatcttatttatgcattacttgcttccttgcatgattgtctttatgcatccccaatcttaattgagaccatacttgtataagatttttgtaatatctttctgctattggatatatttattagttaatatatttatttattttttgctatttaatgtatttatttttttacttggacatgaaactttaaaaaaattcacagattatatttataacctgactagagcaggtgatgtatttttatacagtaaaaaaaaaaaaccttgtaaattctagaagagtggctaggggggttattcatttgtattcaactaaggacatatttactcatgctgatgctctgtgagatatttgaaattgaaccaatgactacttaggatgggttgtggaataagttttgatgtggaattgcacatctaccttacaattactgaccatccccagtagactccccagtcccataattgtgtatcttccagccaggaatcctacacggccagcatgtatttctacaaataaagttttctttgcataacaaaaaaaaaaaaaaaaaaa

SEQ ID NO: 184 - protein, 176 aa, Homo sapiens
MKASSLAFSLLSAAFYLLWTPSTGLKTLNLGSCVIATNLQEIRNGFSEIRGSVQAKDGNIDIRILRRTESLQDTKPANRCCLLRHLLRLYLDRVFKNYQTPDHYTLRKISSLANSFLTIKKDLRLCHAHMTCHCGEEAMKKYSQILSHFEKLEPQAAVVKALGELDILLQWMEETE

SEQ ID NO: 185 - DNA, 2265 bp, Homo sapiens
  source 1..2265: mol_type = unassigned DNA
acagccagagggacgagctagcccgacgatggcccaggggacattgatccgtgtgaccccagagcagcccacccatgccgtgtgtgtgctgggcaccttgactcagcttgacatctgcagctctgcccctgaggactgcacgtccttcagcatcaacgcctccccaggggtggtcgtggatattgcccacggccctccagccaagaagaaatccacaggttcctccacatggcccctggaccctggggtagaggtgaccctgacgatgaaagtggccagtggtagcacaggcgaccagaaggttcagatttcatactacggacccaagactccaccagtcaaagctctactctacctcaccggggtggaaatctccttgtgcgcagacatcacccgcaccggcaaagtgaagccaaccagagctgtgaaagatcagaggacctggacctggggcccttgtggacagggtgccatcctgctggtgaactgtgacagagacaatctcgaatcttctgccatggactgcgaggatgatgaagtgcttgacagcgaagacctgcaggacatgtcgctgatgaccctgagcacgaagacccccaaggacttcttcacaaaccatacactggtgctccacgtggccaggtctgagatggacaaagtgagggtgtttcaggccacacggggcaaactgtcctccaagtgcagcgtagtcttgggtcccaagtggccctctcactacctgatggtccccggtggaaagcacaacatggacttctacgtggaggccctcgctttcccggacaccgacttcccggggctcattaccctcaccatctccctgctggacacgtccaacctggagctccccgaggctgtggtgttccaagacagcgtggtcttccgcgtggcgccctggatcatgacccccaacacccagcccccgcaggaggtgtacgcgtgcagtatttttgaaaatgaggacttcctgaagtcagtgactactctggccatgaaagccaagtgcaagctgaccatctgccctgaggaggagaacatggatgaccagtggatgcaggatgaaatggagatcggctacatccaagccccacacaaaacgctgcccgtggtcttcgactctccaaggaacagaggcctgaaggagtttcccatcaaacgcgtgatgggtccagattttggctatgtaactcgagggccccaaacagggggtatcagtggactggactcctttgggaacctggaagtgagccccccagtcacagtcaggggcaaggaatacccgctgggcaggattctcttcggggacagctgttatcccagcaatgacagccggcagatgcaccaggccctgcaggacttcctcagtgcccagcaggtgcaggcccctgtgaagctctattctgactggctgtccgtgggccacgtggacgagttcctgagctttgtgccagcacccgacaggaagggcttccggctgctcctggccagccccaggtcctgctacaaactgttccaggagcagcagaatgagggccacggggaggccctgctgttcgaagggatcaagaaaaaaaaacagcagaaaataaagaacattctgtcaaacaagacattgagagaacataattcatttgtggagagatgcatcgactggaaccgcgagctgctgaagcgggagctgggcctggccgagagtgacatcattgacatcccgcagctcttcaagctcaaagagttctctaaggcggaagcttttttccccaacatggtgaacatgctggtgctagggaagcacctgggcatccccaagcccttcgggcccgtcatcaacggccgctgctgcctggaggagaaggtgtgttccctgctggagccactgggcctccagtgcaccttcatcaacgacttcttcacctaccacatcaggcatggggaggtgcactgcggcaccaacgtgcgcagaaagcccttctccttcaagtggtggaacatggtgccctgagcccatcttccctggcgtcctctccctcctggccagatgtcgctgggtcctctgcagtgtggcaagcaagagctcttgtgaatattgtggctccctgggggcggccagccctcccagcagtggcttgctttcttctcctgtgatgtcccagtttcccactctgaagatcccaacatggtcctagcactgcacactcagttctgctctaagaagctgcaataaagtttttttaagtcactttgtac

SEQ ID NO: 186 - protein, 663 aa, Homo sapiens
MAQGTLIRVTPEQPTHAVCVLGTLTQLDICSSAPEDCTSFSINASPGVVVDIAHGPPAKKKSTGSSTWPLDPGVEVTLTMKVASGSTGDQKVQISYYGPKTPPVKALLYLTGVEISLCADITRTGKVKPTRAVKDQRTWTWGPCGQGAILLVNCDRDNLESSAMDCEDDEVLDSEDLQDMSLMTLSTKTPKDFFTNHTLVLHVARSEMDKVRVFQATRGKLSSKCSVVLGPKWPSHYLMVPGGKHNMDFYVEALAFPDTDFPGLITLTISLLDTSNLELPEAVVFQDSVVFRVAPWIMTPNTQPPQEVYACSIFENEDFLKSVTTLAMKAKCKLTICPEEENMDDQWMQDEMEIGYIQAPHKTLPVVFDSPRNRGLKEFPIKRVMGPDFGYVTRGPQTGGISGLDSFGNLEVSPPVTVRGKEYPLGRILFGDSCYPSNDSRQMHQALQDFLSAQQVQAPVKLYSDWLSVGHVDEFLSFVPAPDRKGFRLLLASPRSCYKLFQEQQNEGHGEALLFEGIKKKKQQKIKNILSNKTLREHNSFVERCIDWNRELLKRELGLAESDIIDIPQLFKLKEFSKAEAFFPNMVNMLVLGKHLGIPKPFGPVINGRCCLEEKVCSLLEPLGLQCTFINDFFTYHIRHGEVHCGTNVRRKPFSFKWWNMVP

SEQ ID NO: 187 - DNA, 801 bp, Homo sapiens
  source 1..801: mol_type = unassigned DNA
atggtgtgtctgaggctccctggaggctcctgcatggcagttctgacagtgacactgatggtgctgagctccccactggctttggctggggacaccagaccacgtttcttggaggaggttaagtttgagtgtcatttcttcaacgggacggagcgggtgcggttgctggaaagacgcgtccataaccaagaggagtacgcgcgctacgacagcgacgtgggggagtaccgggcggtgacggagctggggcggcctgatgccgagtactggaacagccagaaggacctcctggagcggaggcgtgccgcggtggacacctactgcagacacaactacggggttggtgagagcttcacagtgcagcggcgagttcaacctaaggtgactgtgtatccttcaaagacccagcccctgcagcaccacaacctcctggtctgttctgtgaatggtttctatccaggcagcattgaagtcaggtggttccggaacggccaggaagagaagactggggtggtgtccacgggcctgatccagaatggagactggaccttccagaccctggtgatgctggaaacagttcctcagagtggagaggtttacacctgccaagtggagcacccaagtgtgatgagccctctcacagtggaatggagagcacggtctgaatctgcacagagcaagatgctgagtggagtcgggggctttgtgctgggcctgctcttccttggggccgggctgttcatctacttcaggaatcagaaaggacactctggacttccgccaacaggattcctgagctga

SEQ ID NO: 188 - protein, 266 aa, Homo sapiens [sequence omitted]

SEQ ID NO: 189 - DNA, 3436 bp, Homo sapiens
  source 1..3436: mol_type = unassigned DNA
ggtgtctcggccatgacacacatttgacatgccctccctcaacctacttatagactatttttcttgctctgcagcatggaccaaagagaaattctgcagaagttcctggatgaggcccaaagcaagaaaattactaaagaggagtttgccaatgaatttctgaagctgaaaaggcaatctaccaagtacaaggcagacaaaacctatcctacaactgtggctgagaagcccaagaatatcaagaaaaacagatataaggatattttgccctatgattatagccgggtagaactatccctgataacctctgatgaggattccagctacatcaatgccaacttcattaagggagtttatggacccaaggcttatattgccacccagggtcctttatctacaaccctcctggacttctggaggatgatttgggaatatagtgtccttatcattgttatggcatgcatggagtatgaaatgggaaagaaaaagtgtgagcgctactgggctgagccaggagagatgcagctggaatttggccctttctctgtatcctgtgaagctgaaaaaaggaaatctgattatataatcaggactctaaaagttaagttcaatagtgaaactcgaactatctaccagtttcattacaagaattggccagaccatgatgtaccttcatctatagaccctattcttgagctcatctgggatgtacgttgttaccaagaggatgacagtgttcccatatgcattcactgcagtgctggctgtggaaggactggtgttatttgtgctattgattatacatggatgttgctaaaagatgggagtcaagcaaagcattgtattcctgagaaaaatcacactctccaagcagactcttattctcctaatttaccaaaaagtaccacaaaagcagcaaaaatgatgaaccaacaaaggacaaaaatggaaatcaaagaatcttcttcctttgactttaggacttctgaaataagtgcaaaagaagagctagttttgcaccctgctaaatcaagcacttcttttgactttctggagctaaattacagttttgacaaaaatgctgacacaaccatgaaatggcagacaaaggcatttccaatagttggggagcctcttcagaagcatcaaagtttggatttgggctctcttttgtttgagggatgttctaattctaaacctgtaaatgcagcaggaagatattttaattcaaaggtgccaataacacggaccaaatcaactccttttgaattgatacagcagagagaaaccaaggaggtggacagcaaggaaaacttttcttatttggaatctcaaccacatgattcttgttttgtagagatgcaggctcaaaaagtaatgcatgtttcttcagcagaactgaattattcactgccatatgactctaaacaccaaatacgtaatgcctctaatgtaaagcaccatgactctagtgctcttggtgtatattcttacatacctttagtggaaaatccttatttttcatcatggcctccaagtggtaccagttctaagatgtctcttgatttacctgagaagcaagatggaactgtttttccttcttctctgttgccaacatcctctacatccctcttctcttattacaattcacatgattctttatcactgaattctccaaccaatatttcctcactattgaaccaggagtcagctgtactagcaactgctccaaggatagatgatgaaatcccccctccacttcctgtacggacacctgaatcatttattgtggttgaggaagctggagaattctcaccaaatgttcccaaatccttatcctcagctgtgaaggtaaaaattggaacatcactggaatggggtggaacatctgaaccaaagaaatttgatgactctgtgatacttagaccaagcaagagtgtaaaactccgaagtcctaaatcagaactacatcaagatcgttcttctcccccacctcctctcccagaaagaactctagagtccttctttcttgccgatgaagattgtatgcaggcccaatctatagaaacatattctactagctatcctgacaccatggaaaattcaacatcttcaaaacagacactgaagactcctggaaaaagtttcacaaggagtaagagtttgaaaattttgcgaaacatgaaaaagagtatctgtaattcttgcccaccaaacaagcctgcagaatctgttcagtcaaataactccagctcatttctgaattttggttttgcaaaccgtttttcaaaacccaaaggaccaaggaatccaccaccaacttggaatatttaataaaactccagatttataataatatgggctgcaagtacacctgcaaataaaactactagaatactgctagttaaaataagtgctctatatgcataatatcaaatatgaagatatgctaatgtgttaatagcttttaaaagaaaagcaaaatgccaataagtgccagttttgcattttcatatcatttgcattgagttgaaaactgcaaataaaagtttgtcacttgagcttatgtacagaatgctatatgagaaacacttttagaatggatttatttttcatttttgccagttatttttattttcttttacttttttacataaacataaacttcaaaaggtttgtaagatttggatctcaactaatttctacattgccagaatatactataaaaagttaaaaaaaaaacttactttgtgggttgcaatacaaactgctcttgacaatgactattccctgacagttatttttgcctaaatggagtataccttgtaaatcttcccaaatgttgtggaaaactggaatattaagaaaatgagaaattatatttattagaataaaatgtgcaaataatgacaattatttgaatgtaacaaggaattcaactgaaatcctgataagttttaaccaaagtcattaaattaccaattctagaaaagtaatcaatgaaatataatagctatcttttggtagcaaaagatataaattgtatatgtttatacaggatctttcagatcatgtgcaatttttatctaaccaatcagaaatactagtttaaaatgaatttctatatgaatatggatctgccataagaaaatctagttcaactctaattttatgtagtaaataaattggcaggtaattgtttttacaaagaatccacctgacttcccctaatgcattaaaaatatttttatttaaataactttatttataacttttagaaacatgtagtattgtttaaacatcatttgttcttcagtatttttcatttggaagtccaatagggcaaattgaatgaagtattattatctgtctcttgtagtacaatgtatccaacagacactcaataaactttttggttgttaaaaaaaaaaaaaaa

SEQ ID NO: 190 - protein, 752 aa, Homo sapiens
MDQREILQKFLDEAQSKKITKEEFANEFLKLKRQSTKYKADKTYPTTVAEKPKNIKKNRYKDILPYDYSRVELSLITSDEDSSYINANFIKGVYGPKAYIATQGPLSTTLLDFWRMIWEYSVLIIVMACMEYEMGKKKCERYWAEPGEMQLEFGPFSVSCEAEKRKSDYIIRTLKVKFNSETRTIYQFHYKNWPDHDVPSSIDPILELIWDVRCYQEDDSVPICIHCSAGCGRTGVICAIDYTWMLLKDGSQAKHCIPEKNHTLQADSYSPNLPKSTTKAAKMMNQQRTKMEIKESSSFDFRTSEISAKEELVLHPAKSSTSFDFLELNYSFDKNADTTMKWQTKAFPIVGEPLQKHQSLDLGSLLFEGCSNSKPVNAAGRYFNSKVPITRTKSTPFELIQQRETKEVDSKENFSYLESQPHDSCFVEMQAQKVMHVSSAELNYSLPYDSKHQIRNASNVKHHDSSALGVYSYIPLVENPYFSSWPPSGTSSKMSLDLPEKQDGTVFPSSLLPTSSTSLFSYYNSHDSLSLNSPTNISSLLNQESAVLATAPRIDDEIPPPLPVRTPESFIVVEEAGEFSPNVPKSLSSAVKVKIGTSLEWGGTSEPKKFDDSVILRPSKSVKLRSPKSELHQDRSSPPPPLPERTLESFFLADEDCMQAQSIETYSTSYPDTMENSTSSKQTLKTPGKSFTRSKSLKILRNMKKSICNSCPPNKPAESVQSNNSSSFLNFGFANRFSKPKGPRNPPPTWNI

SEQ ID NO: 191 - DNA, 3825 bp, Homo sapiens
  source 1..3825: mol_type = unassigned DNA
ccggagaggtgttggagagcacaatggctgaacaagtccttcctcaggctttgtatttgagcaatatgcggaaagctgtgaagatacgggagagaactccagaagacatttttaaacctactaatgggatcattcatcattttaaaaccatgcaccgatacacactggaaatgttcagaacttgccagttttgtcctcagtttcgggagatcatccacaaagccctcatcgacagaaacatccaggccaccctggaaagccagaagaaactcaactggtgtcgagaagtccggaagcttgtggcgctgaaaacgaacggtgacggcaattgcctcatgcatgccacttctcagtacatgtggggcgttcaggacacagacttggtactgaggaaggcgctgttcagcacgctcaaggaaacagacacacgcaactttaaattccgctggcaactggagtctctcaaatctcaggaatttgttgaaacggggctttgctatgatactcggaactggaatgatgaatgggacaatcttatcaaaatggcttccacagacacacccatggcccgaagtggacttcagtacaactcactggaagaaatacacatatttgtcctttgcaacatcctcagaaggccaatcattgtcatttcagacaaaatgctaagaagtttggaatcaggttccaatttcgcccctttgaaagtgggtggaatttacttgcctctccactggcctgcccaggaatgctacagataccccattgttctcggctatgacagccatcattttgtacccttggtgaccctgaaggacagtgggcctgaaatccgagctgttccacttgttaacagagaccggggaagatttgaagacttaaaagttcactttttgacagatcctgaaaatgagatgaaggagaagctcttaaaagagtacttaatggtgatagaaatccccgtccaaggctgggaccatggcacaactcatctcatcaatgccgcaaagttggatgaagctaacttaccaaaagaaatcaatctggtagatgattactttgaacttgttcagcatgagtacaagaaatggcaggaaaacagcgagcaggggaggagagaggggcacgcccagaatcccatggaaccttccgtgccccagctttctctcatggatgtaaaatgtgaaacgcccaactgccccttcttcatgtctgtgaacacccagcctttatgccatgagtgctcagagaggcggcaaaagaatcaaaacaaactcccaaagctgaactccaagccgggccctgaggggctccctggcatggcgctcggggcctctcggggagaagcctatgagcccttggcgtggaaccctgaggagtccactggggggcctcattcggccccaccgacagcacccagcccttttctgttcagtgagaccactgccatgaagtgcaggagccccggctgccccttcacactgaatgtgcagcacaacggattttgtgaacgttgccacaacgcccggcaacttcacgccagccacgccccagaccacacaaggcacttggatcccgggaagtgccaagcctgcctccaggatgttaccaggacatttaatgggatctgcagtacttgcttcaaaaggactacagcagaggcctcctccagcctcagcaccagcctccctccttcctgtcaccagcgttccaagtcagatccctcgcggctcgtccggagcccctccccgcattcttgccacagagctggaaacgacgcccctgctggctgcctgtctcaagctgcacggactcctggggacaggacggggacgagcaagtgcagaaaagccggctgcgtgtattttgggactccagaaaacaagggcttttgcacactgtgtttcatcgagtacagagaaaacaaacattttgctgctgcctcagggaaagtcagtcccacagcgtccaggttccagaacaccattccgtgcctggggagggaatgcggcacccttggaagcaccatgtttgaaggatactgccagaagtgtttcattgaagctcagaatcagagatttcatgaggccaaaaggacagaagagcaactgagatcgagccagcgcagagatgtgcctcgaaccacacaaagcacctcaaggcccaagtgcgcccgggcctcctgcaagaacatcctggcctgccgcagcgaggagctctgcatggagtgtcagcatcccaaccagaggatgggccctggggcccaccggggtgagcctgcccccgaagacccccccaagcagcgttgccgggcccccgcctgtgatcattttggcaatgccaagtgcaacggctactgcaacgaatgctttcagttcaagcagatgtatggctaaccggaaacaggtgggtcacctcctgcaagaagtggggcctcgagctgtcagtcatcatggtgctatcctctgaacccctcagctgccactgcaacagtgggcttaagggtgtctgagcaggagaggaaagataagctcttcgtggtgcccacgatgctcaggtttggtaacccgggagtgttcccaggtggccttagaaagcaaagcttgtaactggcaagggatgatgtcagattcagcccaaggttcctcctctcctaccaagcaggaggccaggaacttctttggacttggaaggtgtgcggggactggccgaggcccctgcaccctgcgcatcaggactgcttcatcgtcttggctgagaaagggaaaagacacacaagtcgcgtgggttggagaagccagagccattccacctcccctcccccagcatctctcagagatgtgaagccagatcctcatggcagcgaggccctctgcaagaagctcaaggaagctcagggaaaatggacgtattcagagagtgtttgtagttcatggtttttccctacctgcccggttcctttcctgaggacccggcagaaatgcagaaccatccatggactgtgattctgaggctgctgagactgaacatgttcacattgacagaaaaacaagctgctctttataatatgcaccttttaaaaaattagaatattttactgggaagacgtgtaactctttgggttattactgtctttacttctaaagaagttagcttgaactgaggagtaaaagtgtgtacatatataatatacccttacattatgtatgagggatttttttaaattatattgaaatgctgccctagaagtacaataggaaggctaaataataataacctgttttctggttgttgttggggcatgagcttgtgtatacactgcttgcataaactcaaccagctgcctttttaaagggagctctagtcctttttgtgtaattcactttatttattttattacaaacttcaagattatttaagcgaagatatttcttcagctctggggaaaatgccacagtgttctcctgagagaacatccttgctttgagtcaggctgtgggcaagttcctgaccacagggagtaaattggcctctttgatacacttttgcttgcctccccaggaaagaaggaattgcatccaaggtatacatacatattcatcgatgtttcgtgcttctccttatgaaactccagctatgtaataaaaaactatactctgtgttctgttaatgcctctgagtgtcctacctccttggagatgagatagggaaggagcagggatgagactggcaatggtcacagggaaagatgtggccttttgtgatggttttattttctgttaacactgtgtcctgggggggctgggaagtcccctgcatcccatg

SEQ ID NO: 192 - protein, 790 aa, Homo sapiens
MAEQVLPQALYLSNMRKAVKIRERTPEDIFKPTNGIIHHFKTMHRYTLEMFRTCQFCPQFREIIHKALIDRNIQATLESQKKLNWCREVRKLVALKTNGDGNCLMHATSQYMWGVQDTDLVLRKALFSTLKETDTRNFKFRWQLESLKSQEFVETGLCYDTRNWNDEWDNLIKMASTDTPMARSGLQYNSLEEIHIFVLCNILRRPIIVISDKMLRSLESGSNFAPLKVGGIYLPLHWPAQECYRYPIVLGYDSHHFVPLVTLKDSGPEIRAVPLVNRDRGRFEDLKVHFLTDPENEMKEKLLKEYLMVIEIPVQGWDHGTTHLINAAKLDEANLPKEINLVDDYFELVQHEYKKWQENSEQGRREGHAQNPMEPSVPQLSLMDVKCETPNCPFFMSVNTQPLCHECSERRQKNQNKLPKLNSKPGPEGLPGMALGASRGEAYEPLAWNPEESTGGPHSAPPTAPSPFLFSETTAMKCRSPGCPFTLNVQHNGFCERCHNARQLHASHAPDHTRHLDPGKCQACLQDVTRTFNGICSTCFKRTTAEASSSLSTSLPPSCHQRSKSDPSRLVRSPSPHSCHRAGNDAPAGCLSQAARTPGDRTGTSKCRKAGCVYFGTPENKGFCTLCFIEYRENKHFAAASGKVSPTASRFQNTIPCLGRECGTLGSTMFEGYCQKCFIEAQNQRFHEAKRTEEQLRSSQRRDVPRTTQSTSRPKCARASCKNILACRSEELCMECQHPNQRMGPGAHRGEPAPEDPPKQRCRAPACDHFGNAKCNGYCNECFQFKQMYG

SEQ ID NO: 193 - DNA, 2588 bp, Homo sapiens
  source 1..2588: mol_type = unassigned DNA
gctttctcctagggactgtgaggggcgcttctgactttggacttgagcactgcctgggacctgtgctgagagagcgctagcatgtctcagtggaatcaagtccaacagttagaaatcaagtttttggagcaggtggatcaattctatgatgacaactttcccatggaaattcggcatctgttggcccaatggattgaaaatcaagactgggaggcagcttctaacaatgaaaccatggcaacgattcttcttcaaaacttgttaatacaactggatgaacagttaggtcgtgtttccaaagagaaaaacctactcttgatacacaatctaaaaagaattaggaaggtccttcagggaaaatttcatggaaatccaatgcatgtagctgtggttatttcaaactgtttaagggaagagaggagaatattggctgcagccaacatgcctgtccaggggcctctagagaaatccttacaaagttcttcagtttcagaaagacagaggaatgtggagcacaaagtggctgccattaaaaacagtgtgcagatgacagaacaagataccaaatacttagaagatctgcaagacgaatttgactacaggtataaaacaattcagacaatggatcagagtgacaagaatagtgccatggtgaatcaggaagttttgacactgcaggaaatgcttaacagcctcgatttcaagagaaaggaggctctcagtaaaatgacccaaatcatccatgagacagacctgttaatgaacaccatgctcatagaagagctgcaagactggaagcggcggcagcaaatcgcctgcatcgggggtccactccacaatgggctcgaccagcttcagaactgctttacactattggcagaaagtcttttccaactgagaaggcaattggagaaactagaggagcaatctaccaaaatgacatatgaaggtgatcccattccaatgcaaagaactcacatgctagaaagagtcaccttcttgatctacaaccttttcaagaactcatttgtggttgagcgacagccatgtatgccaacccaccctcagaggccgttggtacttaaaaccctaattcagttcactgtaaaactaaggctactaataaaattgccagaactaaactatcaggtaaaggttaaggcatcaattgacaagaatgtttcaactctaagcaaccgaagatttgtactttgtggaactaatgtcaaagccatgtctattgaagaatcttccaatgggagtctctcagtagaatttcgacatttgcaaccaaaggaaatgaagtccagtgctggaggtaaaggaaatgagggctgtcacatggtgactgaagaacttcattccataacgtttgaaacacagatctgcctctatggcctgaccatagatttggagaccagctcattgcctgtggtgatgatttccaatgtcagtcagttacctaatgcttgggcatccatcatttggtacaacgtgtcaaccaacgattcccagaacttggttttctttaataatcctccacctgccacattgagtcaactactggaggtgatgagctggcagttttcatcgtacgttggtcgtggtcttaactcagatcaactccatatgctggcagagaagcttacagtccaatctagctacagtgatggtcacctcacctgggccaagttctgcaaggaacatttacctggtaaatcatttaccttttggacatggcttgaagcaatattggatctaattaagaaacacattcttcccctttggattgatgggtatgtcatgggctttgttagcaaagagaaggaacggctgttgctaaaggataaaatgcctggcacctttttattaagattcagtgaaagccatctcggaggaataactttcacctgggtggaccattctgaaagtggggaagtgagattccactctgtagaaccctacaataaaggccggttgtctgctctgccattcgctgacatcctgcgagactacaaagttattatggctgaaaacattcctgaaaaccctctgaagtacctatatcctgacattcccaaagacaaagccttcggtaaacactacagctctcagccttgcgaagtttcaagaccaacagaaaggggtgacaaaggttatgttccttctgtttttatccccatctcaacaatccgaagtgattcaacagagccacattctccatcagaccttcttcccatgtctccaagtgtgtatgcggtgttgagagaaaacctgagtcccacaacaattgaaactgcaatgaagtctccttattctgctgaatgacaggataaactctgacgcaccaagaaaggaagcaaatgaaaaagtttaaagactgttctttgcccaataaccacattttatttcttcagctttgtaaataccaggttctaggaaatgtttgacatctgaagctctcttcacactcccgtggcactcctcaattgggagtgttgtgactgaaatgcttgaaaccaaagcttcagataaacttgcaagataagacaactttaagaaaccagtgttaataacaatattaacag

SEQ ID NO: 194 - protein, 748 aa, Homo sapiens
MSQWNQVQQLEIKFLEQVDQFYDDNFPMEIRHLLAQWIENQDWEAASNNETMATILLQNLLIQLDEQLGRVSKEKNLLLIHNLKRIRKVLQGKFHGNPMHVAVVISNCLREERRILAAANMPVQGPLEKSLQSSSVSERQRNVEHKVAAIKNSVQMTEQDTKYLEDLQDEFDYRYKTIQTMDQSDKNSAMVNQEVLTLQEMLNSLDFKRKEALSKMTQIIHETDLLMNTMLIEELQDWKRRQQIACIGGPLHNGLDQLQNCFTLLAESLFQLRRQLEKLEEQSTKMTYEGDPIPMQRTHMLERVTFLIYNLFKNSFVVERQPCMPTHPQRPLVLKTLIQFTVKLRLLIKLPELNYQVKVKASIDKNVSTLSNRRFVLCGTNVKAMSIEESSNGSLSVEFRHLQPKEMKSSAGGKGNEGCHMVTEELHSITFETQICLYGLTIDLETSSLPVVMISNVSQLPNAWASIIWYNVSTNDSQNLVFFNNPPPATLSQLLEVMSWQFSSYVGRGLNSDQLHMLAEKLTVQSSYSDGHLTWAKFCKEHLPGKSFTFWTWLEAILDLIKKHILPLWIDGYVMGFVSKEKERLLLKDKMPGTFLLRFSESHLGGITFTWVDHSESGEVRFHSVEPYNKGRLSALPFADILRDYKVIMAENIPENPLKYLYPDIPKDKAFGKHYSSQPCEVSRPTERGDKGYVPSVFIPISTIRSDSTEPHSPSDLLPMSPSVYAVLRENLSPTTIETAMKSPYSAE

SEQ ID NO: 195 - DNA, 1125 bp, Homo sapiens
  source 1..1125: mol_type = unassigned DNA
atgagcggggaatcaatgaatttcagcgatgttttcgactccagtgaagattattttgtgtcagtcaatacttcatattactcagttgattctgagatgttactgtgctccttgcaggaggtcaggcagttctccaggctatttgtaccgattgcctactccttgatctgtgtctttggcctcctggggaatattctggtggtgatcacctttgctttttataagaaggccaggtctatgacagacgtctatctcttgaacatggccattgcagacatcctctttgttcttactctcccattctgggcagtgagtcatgccactggtgcgtgggttttcagcaatgccacgtgcaagttgctaaaaggcatctatgccatcaactttaactgcgggatgctgctcctgacttgcattagcatggaccggtacatcgccattgtacaggcgactaagtcattccggctccgatccagaacactaccgcgcagcaaaatcatctgccttgttgtgtgggggctgtcagtcatcatctccagctcaacttttgtcttcaaccaaaaatacaacacccaaggcagcgatgtctgtgaacccaagtaccagactgtctcggagcccatcaggtggaagctgctgatgttggggcttgagctactctttggtttctttatccctttgatgttcatgatattttgttacacgttcattgtcaaaaccttggtgcaagctcagaattctaaaaggcacaaagccatccgtgtaatcatagctgtggtgcttgtgtttctggcttgtcagattcctcataacatggtcctgcttgtgacggctgcaaatttgggtaaaatgaaccgatcctgccagagcgaaaagctaattggctatacgaaaactgtcacagaagtcctggctttcctgcactgctgcctgaaccctgtgctctacgcttttattgggcagaagttcagaaactactttctgaagatcttgaaggacctgtggtgtgtgagaaggaagtacaagtcctcaggcttctcctgtgccgggaggtactcagaaaacatttctcggcagaccagtgagaccgcagataacgacaatgcgtcgtccttcactatgtga

SEQ ID NO: 196 - protein, 374 aa, Homo sapiens
MSGESMNFSDVFDSSEDYFVSVNTSYYSVDSEMLLCSLQEVRQFSRLFVPIAYSLICVFGLLGNILVVITFAFYKKARSMTDVYLLNMAIADILFVLTLPFWAVSHATGAWVFSNATCKLLKGIYAINFNCGMLLLTCISMDRYIAIVQATKSFRLRSRTLPRSKIICLVVWGLSVIISSSTFVFNQKYNTQGSDVCEPKYQTVSEPIRWKLLMLGLELLFGFFIPLMFMIFCYTFIVKTLVQAQNSKRHKAIRVIIAVVLVFLACQIPHNMVLLVTAANLGKMNRSCQSEKLIGYTKTVTEVLAFLHCCLNPVLYAFIGQKFRNYFLKILKDLWCVRRKYKSSGFSCAGRYSENISRQTSETADNDNASSFTM

SEQ ID NO: 197 - DNA, 2258 bp, Homo sapiens
  source 1..2258: mol_type = unassigned DNA
ctcctccagctcttcctgtcccgctgttgcaacactgcctcactcttcccctcccaccttctctcccctcctctctgctttaattttctcagaattctctggactgaggctccagttctggcctttggggttcaagatcactgggaccaggccgtgatctctatgcccgagtctcaaccctcaactgtcaccccaaggcacttgggacgtcctggacagaccgagtcccgggaagccccagcactgccgctgccacactgccctgagcccaaatgggggagtgagaggccatagctgtctggcatgggcctctccaccgtgcctgacctgctgctgccactggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccccagattgagaatgttaagggcactgaggactcaggcaccacagtgctgttgcccctggtcattttctttggtctttgccttttatccctcctcttcattggtttaatgtatcgctaccaacggtggaagtccaagctctactccattgtttgtgggaaatcgacacctgaaaaagagggggagcttgaaggaactactactaagcccctggccccaaacccaagcttcagtcccactccaggcttcacccccaccctgggcttcagtcccgtgcccagttccaccttcacctccagctccacctatacccccggtgactgtcccaactttgcggctccccgcagagaggtggcaccaccctatcagggggctgaccccatccttgcgacagccctcgcctccgaccccatccccaacccccttcagaagtgggaggacagcgcccacaagccacagagcctagacactgatgaccccgcgacgctgtacgccgtggtggagaacgtgcccccgttgcgctggaaggaattcgtgcggcgcctagggctgagcgaccacgagatcgatcggctggagctgcagaacgggcgctgcctgcgcgaggcgcaatacagcatgctggcgacctggaggcggcgcacgccgcggcgcgaggccacgctggagctgctgggacgcgtgctccgcgacatggacctgctgggctgcctggaggacatcgaggaggcgctttgcggccccgccgccctcccgcccgcgcccagtcttctcagatgaggctgcgcccctgcgggcagctctaaggaccgtcctgcgagatcgccttccaaccccacttttttctggaaaggaggggtcctgcaggggcaagcaggagctagcagccgcctacttggtgctaacccctcgatgtacatagcttttctcagctgcctgcgcgccgccgacagtcagcgctgtgcgcgcggagagaggtgcgccgtgggctcaagagcctgagtgggtggtttgcgaggatgagggacgctatgcctcatgcccgttttgggtgtcctcaccagcaaggctgctcgggggcccctggttcgtccctgagcctttttcacagtgcataagcagttttttttgtttttgttttgttttgttttgtttttaaatcaatcatgttacactaatagaaacttggcactcctgtgccctctgcctggacaagcacatagcaagctgaactgtcctaaggcaggggcgagcacggaacaatggggccttcagctggagctgtggacttttgtacatacactaaaattctgaagttaaagctctgctcttggaaaaaaaaaaaaaaaaaaaaaaaaaaaaaaaaa

SEQ ID NO: 198 - protein, 455 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 199 - DNA, 2394 bp, Homo sapiens
  source 1..2394: mol_type = unassigned DNA
gaattcggcgcagcggagcctggagagaaggcgctgggctgcgagggcgcgagggcgcgagggcagggggcaaccggaccccgcccgcacccatggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaagtgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcagggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcactggcgacttcgctcttccagttggactgattgtgggtgtgacagccttgggtctactaataataggagtggtgaactgtgtcatcatgacccaggtgaaaaagaagcccttgtgcctgcagagagaagccaaggtgcctcacttgcctgccgataaggcccggggtacacagggccccgagcagcagcacctgctgatcacagcgccgagctccagcagcagctccctggagagctcggccagtgcgttggacagaagggcgcccactcggaaccagccacaggcaccaggcgtggaggccagtggggccggggaggcccgggccagcaccgggagctcagattcttcccctggtggccatgggacccaggtcaatgtcacctgcatcgtgaacgtctgtagcagctctgaccacagctcacagtgctcctcccaagccagctccacaatgggagacacagattccagcccctcggagtccccgaaggacgagcaggtccccttctccaaggaggaatgtgcctttcggtcacagctggagacgccagagaccctgctggggagcaccgaagagaagcccctgccccttggagtgcctgatgctgggatgaagcccagttaaccaggccggtgtgggctgtgtcgtagccaaggtgggctgagccctggcaggatgaccctgcgaaggggccctggtccttccaggcccccaccactaggactctgaggctctttctgggccaagttcctctagtgccctccacagccgcagcctccctctgacctgcaggccaagagcagaggcagcgggttgtggaaagcctctgctgccatggtgtgtccctctcggaaggctggctgggcatggacgttcggggcatgctggggcaagtccctgactctctgtgacctgccccgcccagctgcacctgccagcctggcttctggagcccttgggttttttgtttgtttgtttgtttgtttgtttgtttctccccctgggctctgccccagctctggcttccagaaaaccccagcatccttttctgcagaggggctttctggagaggagggatgctgcctgagtcacccatgaagacaggacagtgcttcagcctgaggctgagactgcgggatggtcctggggctctgtgcagggaggaggtggcagccctgtagggaacggggtccttcaagttagctcaggaggcttggaaagcatcacctcaggccaggtgcagtccctcacgcctatgatcccagcactttgggaggctgaggcgggtggatcacctgaggttaggagttcgagaccagcctggccaacatggtaaaaccccatctctactaaaaatacagaaattagccgggcgtggtggcgggcacctatagtcccagctactcagaagcctgaggctgggaaatcgtttgaacccgggaagcggaggttgcagggagccgagatcacgccactgcactccagcctgggcgacagagcgagagtctgtctcaaaagaaaaaaaaaaaaaaccgaattc

SEQ ID NO: 200 - protein, 461 aa, Homo sapiens [sequence omitted]

SEQ ID NO: 201 - DNA, 1240 bp, Homo sapiens
  source 1..1240: mol_type = unassigned DNA
gacgtgaagagtttaaagaaagagtattcaaacgaaaatgcagttgtgaagagaatgcagtctcttcaacttgattgtgtggcagtaccttcaagccggtcaaattcagccacagaacagcctggttcactgcacagttcccagggacttgggatgggtcctgtggaggagtcctggtttgctccttccctggagcacccacaagaagagaatgagcccagcctgcagagtaaactccaagacgaagccaactaccatctttatggcagccgcatggacaggcagacgaaacagcagcccagacagaatgtggcttacaacagagaggaggaaaggagacgcagggtctcccatgacccttttgcacagcaaagaccttacgagaattttcagaatacagagggaaaaggcactgtttattccagtgcagccagtcatggtaatgcagtgcaccagccatcagggctcaccagccaacctcaagtactgtatcagaacaatggattatatagctcacatggctttggaacaagaccactggatccaggaacagcaggtcccagagtttggtacaggccaattccaagtcatatgcctagtctgcataatatcccagtgcctgagaccaactatctaggaaattctcccaccatgccattcagctccttgccaccaacagatgaatctataaaatataccatatacaatagtactggcattcagattggagcctacaattatatggagattggtgggacgagttcatcactactagacagcacaaatacgaacttcaaagaagagccagctgctaagtaccaagctatctttgataataccactagtctgacggataaacacctggacccaatcagggaaaatctgggaaagcactggaaaaactgtgcccgtaaactgggcttcacacagtctcagattgatgaaattgaccatgactatgagcgagatggactgaaagaaaaggtttaccagatgctccaaaagtgggtgatgagggaaggcataaagggagccacggtggggaagctggcccaggcgctccaccagtgttccaggatcgaccttctgagcagcttgatttacgtcagccagaactaaccctggatgggctacggcagctgaagtggacgcctcacttagtggataaccccagaaagttggctgcctcagagcattcagaattctgtcctcactgataggggttctgtgtctgcagaaa

SEQ ID NO: 202 - protein, 372 aa, Homo sapiens
DVKSLKKEYSNENAVVKRMQSLQLDCVAVPSSRSNSATEQPGSLHSSQGLGMGPVEESWFAPSLEHPQEENEPSLQSKLQDEANYHLYGSRMDRQTKQQPRQNVAYNREEERRRRVSHDPFAQQRPYENFQNTEGKGTVYSSAASHGNAVHQPSGLTSQPQVLYQNNGLYSSHGFGTRPLDPGTAGPRVWYRPIPSHMPSLHNIPVPETNYLGNSPTMPFSSLPPTDESIKYTIYNSTGIQIGAYNYMEIGGTSSSLLDSTNTNFKEEPAAKYQAIFDNTTSLTDKHLDPIRENLGKHWKNCARKLGFTQSQIDEIDHDYERDGLKEKVYQMLQKWVMREGIKGATVGKLAQALHQCSRIDLLSSLIYVSQN

SEQ ID NO: 203 - DNA, 1496 bp, Homo sapiens
  source 1..1496: mol_type = unassigned DNA
gcacacccggaagcggcggagtagagcggagcctggcgggcgtgggaacccaggccccgccgaggcggccaggaggtgagatggcagctgggcaaaatgggcacgaagagtgggtgggcagcgcatacctgtttgtggagtcctcgctggacaaggtggtcctgtcggatgcctacgcgcacccccagcagaaggtggcagtgtacagggctctgcaggctgccttggcagagagcggcgggagcccggacgtgctgcagatgctgaagatccaccgcagcgacccgcagctgatcgtgcagctgcgattctgcgggcggcagccctgtggccgcttcctccgcgcctaccgcgagggggcgctgcgcgccgcgctgcagaggagcctggcggccgcgctcgcccagcactcggtgccgctgcaactggagctgcgcgccggcgccgagcggctggacgctttgctggcggacgaggagcgctgtttgagttgcatcctagcccagcagcccgaccggctccgggatgaagaactggctgagctggaggatgcgctgcgaaatctgaagtgcggctcgggggcccggggtggcgacggggaggtcgcttcggcccccttgcagcccccggtgccctctctgtcggaggtgaagccgccgccgccgccgccacctgcccagacttttctgttccagggtcagcctgtagtgaatcggccgctgagcctgaaggaccaacagacgttcgcgcgctctgtgggtctcaaatggcgcaaggtggggcgctcactgcagcgaggctgccgggcgctgcgggacccggcgctggactcgctggcctacgagtacgagcgcgagggactgtacgagcaggccttccagctgctgcggcgcttcgtgcaggccgagggccgccgcgccacgctgcagcgcctggtggaggcactcgaggagaacgagctcaccagcctggcagaggacttgctgggcctgaccgatcccaatggcggcctggcctagaccaggggtgcagccagcttttggagaacctggatggccttagggttccttctgcggctattgctgaacccctgtccatccacgggaccctgaaactccacttggcctatctgctggacctgctggggcagagttgattgccttccccaggagccagaccactgggggtgcatcattggggattctgcctcaggtactttgatagagtgtggggtgggggggacctgctttggagatcagcctcaccttctcccatcccagaagcggggcttacagccagcccttacagtttcactcatgaagcaccttgatctttggtgtcctggacttcatcctgggtgctgcagatactgcagtgaagtaaaacaggaatcaatcttgcctgcccccagctcacactcagcgtgggaccccgaatgttaagcaatgataataaagtataacacggattttgatgtgagaaaaaaaaaaaaaaaa

SEQ ID NO: 204 - protein, 312 aa, Homo sapiens
MAAGQNGHEEWVGSAYLFVESSLDKVVLSDAYAHPQQKVAVYRALQAALAESGGSPDVLQMLKIHRSDPQLIVQLRFCGRQPCGRFLRAYREGALRAALQRSLAAALAQHSVPLQLELRAGAERLDALLADEERCLSCILAQQPDRLRDEELAELEDALRNLKCGSGARGGDGEVASAPLQPPVPSLSEVKPPPPPPPAQTFLFQGQPVVNRPLSLKDQQTFARSVGLKWRKVGRSLQRGCRALRDPALDSLAYEYEREGLYEQAFQLLRRFVQAEGRRATLQRLVEALEENELTSLAEDLLGLTDPNGGLA

SEQ ID NO: 205 - DNA, 4363 bp, Homo sapiens
  source 1..4363: mol_type = unassigned DNA
gcaggctgctggagaaggcgcacctgctgcaggtgctcccggccgccccggaccagcgagcgcgggcactgcggcggggaggatgctgcgcgagcggaccgtgcggctgcagtacgggagccgcgtggaggcggtgtacgtgctgggcacctacctctggaccgatgtctacagcgcggccccagccggggcccaaaccttcagcctgaagcactcggaacacgtgtgggtggaggtggtgcgtgatggggaggctgaggaggtggccaccaatggcaagcagcgctggcttctctcgcccagcaccaccctgcgggtcaccatgagccaggcgagcaccgaggccagcagtgacaaggtcaccgtcaactactatgacgaggaagggagcattcccatcgaccaggcggggctcttcctcacagccattgagatctccctggatgtggacgcagaccgggatggtgtggtggagaagaacaacccaaagaaggcatcctggacctggggccccgagggccagggggccatcctgctggtgaactgtgaccgagagacaccctggttgcccaaggaggactgccgtgatgagaaggtctacagcaaggaagatctcaaggacatgtcccagatgatcctgcggaccaaaggccccgaccgcctccccgccggatacgagatagttctgtacatttccatgtcagactcagacaaagtgggcgtgttctacgtggagaacccgttcttcggccaacgctatatccacatcctgggccggcggaagctctaccatgtggtcaagtacacgggtggctccgcggagctgctgttcttcgtggaaggcctctgtttccccgacgagggcttctcaggcctggtctccatccatgtcagcctgctggagtacatggcccaggacattcccctgactcccatcttcacggacaccgtgatattccggattgctccgtggatcatgacccccaacatcctgcctcccgtgtcggtgtttgtgtgctgcatgaaggataattacctgttcctgaaagaggtgaagaaccttgtggagaaaaccaactgtgagctgaaggtctgcttccagtacctaaaccgaggcgatcgctggatccaggatgaaattgagtttggctacatcgaggccccccataaaggcttccccgtggtgctggactctccccgagatggaaacctaaaggacttccctgtgaaggagctcctgggcccagattttggctacgtgacccgggagcccctctttgagtctgtcaccagccttgactcatttggaaacctggaggtcagtcccccagtgaccgtgaacggcaagacatacccgcttggccgcatcctcatcgggagcagctttcctctgtctggtggtcggaggatgaccaaggtggtgcgtgacttcctgaaggcccagcaggtgcaggcgcccgtggagctctactcagactggctgactgtgggccacgtggatgagttcatgtcctttgtccccatccccggcacaaagaaattcctgctactcatggccagcacctcggcctgctacaagctcttccgagagaagcagaaggacggccatggagaggccatcatgttcaaaggcttgggtgggatgagcagcaagcgaatcaccatcaacaagattctgtccaacgagagccttgtgcaggagaacctgtacttccagcgctgcctagactggaaccgtgacatcctcaagaaggagctgggactgacagagcaggacatcattgacctgcccgctctgttcaagatggacgaggaccaccgtgccagagccttcttcccaaacatggtgaacatgatcgtgctggacaaggacctgggcatccccaagccattcgggccacaggttgaggaggaatgctgcctggagatgcacgtgcgtggcctcctggagcccctgggcctcgaatgcaccttcatcgacgacatttctgcctaccacaaatttctgggggaagtccactgtggcaccaacgtccgcaggaagcccttcaccttcaagtggtggcacatggtgccctgacctgccaggggccctggcgtttgcctccttcgcttagttctccagaccctccctcacacgcccagagccttctgctgacatggactggacagccccgctgggagacctttgggacgtggggtggaatttggggtatctgtgccttgccctccctgagaggggcctcagtgtcctctgaagccatccccagtgagcctcgactctgtccctgctgaaaatagctgggccagtgtctctgtagccctgacataaggaacagaacacaacaaaacacagcaaaccatgtgcccaaactgctccccaaagaattttgagtctctaatctgacactgaatgaggggagaagggaaggagattctgggattgccagttcttccagcagccatgctctgaaaatcaaggtagaatccatggaaagggaccccaggaccccgggaccctagacgtatcttgaactgccatcgtcatttcaaatacatctccctcagggtttccaggtggccacccccaattattcattccttaccaacctctcaaatcctcttggctttctctctgcagtgtggacactgttggctagtcctccccactccctgagggtccagtaagttagcttagaaccttcctggaaacatttcatctgagcaggtttccccacgtgtgggatgctccttttgcctcatctgtctcagggatgcaggctcccccgcatgcatggggatttctccccagaccagcatacttgtgacctgagagttcaatgcgtaaagatgcccctggtcagccatatccatcttctcttgcctggtccttgattctctggccgctccctgaccttcctccttccactgccttgactttcttcctttttattcctggtgccatctgtccaggcagctagacaagaacttgttcgccagcagccagattcaggccttcccaggggcataataagtgaccagcccctcctctccggacatcagatccaacacataaggaccctggcctaccctccagcccaacagccagttctgggtcagctgccaacttaggggtggtttgattatcccattgaaattcaccagtgcctttgccaaagaccctctcatttggacatacccagattcattccctggctccaactgaaaagactcagtttcaatcgttaaaagttcctttagggccagaagaataaatgaattataatcccattttgaagaaccgatttataaccaatgaaaaggttataatgtaatttatattcttggaggaacaagattttcatttgggattatttccttcaaccattcaacaaacatttgttgtatgccactaagcgccaggcacggcgttgggctctgcaaacacagtggttagtagcagtctggacctggtccctactggcatggaacccatcactccccaacatgcaaagcccacatttaaaggccagcctctgccccttcagtgatgcgctctttagaaatgccagtccactatattcagaaatccgcagggcacaaaacttccagcaagtcactgttgtggtgaaatgggcagtgggggtggggggtcttctttaaacaggcccccttcccatctacctagccagtacccatccaatgagtccccagagcctccagaagctgttgtctcctctctggggacagcagctcctgcctttggaggccaaagccccagatctctccagccccagagctgaaaacaccaagtgcctatttgagggtgtctgtctggagacttagagtttgtcatgtgtgtgtgtgtgtttggttaatgtgggtttatgggttttctttcttttttttctttttttttttagtctacattagggggaagtgagcgcctcccatgtgcagacagtgtgtctttatagatttttctaaggctttccccaatgatgtcggtaatttctgatgtttctgaagttcccaggactcacacacccgttcccatctcacttgcccacccagtgtgacaaccctcggtgtggatatacccccgtggactcatggctcttccccacccccactttctataaatgtaggcctagaatacgcttctctgttgcaaaactcagctaagttcctgcttccaccttgatgttgaaatatcttatgtaagagggcaggggatgtcgtgaagatggcaagaagaacacagtttcaaatttctggaaaagagcctgtggtggagatctaaagatgtttagggaagagctcgactaaagaacaatgaaataaatggtccaaggggaagtca

SEQ ID NO: 206 - protein, 665 aa, Homo sapiens
MLRERTVRLQYGSRVEAVYVLGTYLWTDVYSAAPAGAQTFSLKHSEHVWVEVVRDGEAEEVATNGKQRWLLSPSTTLRVTMSQASTEASSDKVTVNYYDEEGSIPIDQAGLFLTAIEISLDVDADRDGVVEKNNPKKASWTWGPEGQGAILLVNCDRETPWLPKEDCRDEKVYSKEDLKDMSQMILRTKGPDRLPAGYEIVLYISMSDSDKVGVFYVENPFFGQRYIHILGRRKLYHVVKYTGGSAELLFFVEGLCFPDEGFSGLVSIHVSLLEYMAQDIPLTPIFTDTVIFRIAPWIMTPNILPPVSVFVCCMKDNYLFLKEVKNLVEKTNCELKVCFQYLNRGDRWIQDEIEFGYIEAPHKGFPVVLDSPRDGNLKDFPVKELLGPDFGYVTREPLFESVTSLDSFGNLEVSPPVTVNGKTYPLGRILIGSSFPLSGGRRMTKVVRDFLKAQQVQAPVELYSDWLTVGHVDEFMSFVPIPGTKKFLLLMASTSACYKLFREKQKDGHGEAIMFKGLGGMSSKRITINKILSNESLVQENLYFQRCLDWNRDILKKELGLTEQDIIDLPALFKMDEDHRARAFFPNMVNMIVLDKDLGIPKPFGPQVEEECCLEMHVRGLLEPLGLECTFIDDISAYHKFLGEVHCGTNVRRKPFTFKWWHMVP

SEQ ID NO: 207 - DNA, 3189 bp, Homo sapiens
  source 1..3189: mol_type = unassigned DNA
agtgttggggttggcggccacagctaagtccaacaccagcatgtcgctgcagagaatcgtgcgtgtgtccctggagcatcccaccagcgcggtgtgtgtggctggcgtggagaccctcgtggacatttatgggtcagtgcctgagggcacagaaatgtttgaggtctatgggacgcctggcgtggacatctacatctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctacctcacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacggccttcttccctgacttggtgaacatgctggtgctggggaagcacctgggcatccccaagccctttgggcccatcatcaatggctgctgctgcctggaggagaaggtgcggtccctgctggagccgctgggcctccactgcaccttcattgatgacttcactccataccacatgctgcatggggaggtgcactgtggcaccaatgtgtgcagaaagcccttctctttcaagtggtggaacatggtgccctgagacagctcccacccaccatcctgtccccctggggcgggcattggcccaggtggtggagacagagacaggcccctgaacgataagcaccaagagaccccaaggctccagatggaacactgagggtgaccgtccctctcagaagccttttccctggaagtgtccatgcctcacctgcaacccatgtggttctcagacttgaatcttctcggccccccaaaaagaaggacctcatttcttatagcctctcctgtgattcaacacaacccatggagatgtccccttctcactctgaaatcatccatttggggacaaatccacattggggtctagaaacatccacgtatctcatcagccatcttgtcctgtgcatcctaacagaggaaggatccatgattctgctttggtccaattgcttcctctctgcagaggaacaaccctaaaaccagaccactccacgcaggacaggcaggagagattcttcctaaagcctcccccataaaaagggagctgtggatccacttagatcagggcggaaccatctttcacccggccaagctcctgcccagatgttgaccctcacccagcgtgagctgtcacatagtaggagcttctagatgcatgtggaagcaatgagagttgtcccttagccttataaactccccatgatctgacatgcagaaatccagccttgtccagaatcctcctggaatttcttggagacgaaagtatctgggggattgttgggtactagggagactgggtacaagggtgaaaagtagttcccataatacacatggttgactatggtgatccaccttgtgatggttaatattaggtgtctggagaaggttgcttcattggccctgggacttctctctgcaggaggagagaacgctgcctctcctctggattggtctcaggctctctgttggcctttggtcagcgtttccacatcctgctctgctgcaggagagggggctaaggggctggatccaccaaggcagctcacagcgggaaaactctgggaatgaaccactgaattcaggggatgggggtgggggggcggttctcgaggtgtgtgccagctacacgtgtgttctgtatgggtccagctgcgtttccatcactcgctaataaatcaacagaaacacaaa

SEQ ID NO: 208 - protein, 664 aa, Homo sapiens
MSLQRIVRVSLEHPTSAVCVAGVETLVDIYGSVPEGTEMFEVYGTPGVDIYISPNMERGRERADTRRWRFDATLEIIVVMNSPSNDLNDSHVQISYHSSHEPLPLAYAVLYLTCVDISLDCDLNCEGRQDRNFVDKRQWVWGPSGYGGILLVNCDRDDPSCDVQDNCDQHVHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATAFFPDLVNMLVLGKHLGIPKPFGPIINGCCCLEEKVRSLLEPLGLHCTFIDDFTPYHMLHGEVHCGTNVCRKPFSFKWWNMVP

SEQ ID NO: 209 - DNA, 1296 bp, Homo sapiens
  source 1..1296: mol_type = unassigned DNA
atgcccaaccccaggcctggcaagccctcggccccttccttggcccttggcccatccccaggagcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcagctgcccacactgcccctagtcatggtggcaccctccggggcacggctgggccccttgccccacttacaggcactcctccaggacaggccacatttcatgcaccagctctcaacggtggatgcccacgcccggacccctgtgctgcaggtgcaccccctggagagcccagccatgatcagcctcacaccacccaccaccgccactggggtcttctccctcaaggcccggcctggcctcccacctgggatcaacgtggccagcctggaatgggtgtccagggagccggcactgctctgcaccttcccaaatcccagtgcacccaggaaggacagcaccctttcggctgtgccccagagctcctacccactgctggcaaatggtgtctgcaagtggcccggatgtgagaaggtcttcgaagagccagaggacttcctcaagcactgccaggcggaccatcttctggatgagaagggcagggcacaatgtctcctccagagagagatggtacagtctctggagcagcagctggtgctggagaaggagaagctgagtgccatgcaggcccacctggctgggaaaatggcactgaccaaggcttcatctgtggcatcatccgacaagggctcctgctgcatcgtagctgctggcagccaaggccctgtcgtcccagcctggtctggcccccgggaggcccctgacagcctgtttgctgtccggaggcacctgtggggtagccatggaaacagcacattcccagagttcctccacaacatggactacttcaagttccacaacatgcgaccccctttcacctacgccacgctcatccgctgggccatcctggaggctccagagaagcagcggacactcaatgagatctaccactggttcacacgcatgtttgccttcttcagaaaccatcctgccacctggaagaacgccatccgccacaacctgagtctgcacaagtgctttgtgcgggtggagagcgagaagggggctgtgtggaccgtggatgagctggagttccgcaagaaacggagccagaggcccagcaggtgttccaaccctacacctggcccctga

SEQ ID NO: 210 - protein, 431 aa, Homo sapiens
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 211 - DNA, 3216 bp, Homo sapiens
  source 1..3216: mol_type = unassigned DNA
ggcagtttcctggctgaacacgccagcccaatacttaaagagagcaactcctgactccgatagagactggatggacccacaagggtgacagcccaggcggaccgatcttcccatcccacatcctccggcgcgatgccaaaaagaggctgacggcaactgggccttctgcagagaaagacctccgcttcactgccccggctggtcccaagggtcaggaagatggattcatacctgctgatgtggggactgctcacgttcatcatggtgcctggctgccaggcagagctctgtgacgatgacccgccagagatcccacacgccacattcaaagccatggcctacaaggaaggaaccatgttgaactgtgaatgcaagagaggtttccgcagaataaaaagcgggtcactctatatgctctgtacaggaaactctagccactcgtcctgggacaaccaatgtcaatgcacaagctctgccactcggaacacaacgaaacaagtgacacctcaacctgaagaacagaaagaaaggaaaaccacagaaatgcaaagtccaatgcagccagtggaccaagcgagccttccaggtcactgcagggaacctccaccatgggaaaatgaagccacagagagaatttatcatttcgtggtggggcagatggtttattatcagtgcgtccagggatacagggctctacacagaggtcctgctgagagcgtctgcaaaatgacccacgggaagacaaggtggacccagccccagctcatatgcacaggtgaaatggagaccagtcagtttccaggtgaagagaagcctcaggcaagccccgaaggccgtcctgagagtgagacttcctgcctcgtcacaacaacagattttcaaatacagacagaaatggctgcaaccatggagacgtccatatttacaacagagtaccaggtagcagtggccggctgtgttttcctgctgatcagcgtcctcctcctgagtgggctcacctggcagcggagacagaggaagagtagaagaacaatctagaaaaccaaaagaacaagaatttcttggtaagaagccgggaacagacaacagaagtcatgaagcccaagtgaaatcaaaggtgctaaatggtcgcccaggagacatccgttgtgcttgcctgcgttttggaagctctgaagtcacatcacaggacacggggcagtggcaaccttgtctctatgccagctcagtcccatcagagagcgagcgctacccacttctaaatagcaatttcgccgttgaagaggaagggcaaaaccactagaactctccatcttattttcatgtatatgtgttcattaaagcatgaatggtatggaactctctccaccctatatgtagtataaagaaaagtaggtttacattcatctcattccaacttcccagttcaggagtcccaaggaaagccccagcactaacgtaaatacacaacacacacactctaccctatacaactggacattgtctgcgtggttcctttctcagccgcttctgactgctgattctcccgttcacgttgcctaataaacatccttcaagaactctgggctgctacccagaaatcattttacccttggctcaatcctctaagctaacccccttctactgagccttcagtcttgaatttctaaaaaacagaggccatggcagaataatctttgggtaacttcaaaacggggcagccaaacccatgaggcaatgtcaggaacagaaggatgaatgaggtcccaggcagagaatcatacttagcaaagttttacctgtgcgttactaattggcctctttaagagttagtttctttgggattgctatgaatgataccctgaatttggcctgcactaatttgatgtttacaggtggacacacaaggtgcaaatcaatgcgtacgtttcctgagaagtgtctaaaaacaccaaaaagggatccgtacattcaatgtttatgcaaggaaggaaagaaagaaggaagtgaagagggagaagggatggaggtcacactggtagaacgtaaccacggaaaagagcgcatcaggcctggcacggtggctcaggcctataaccccagctccctaggagaccaaggcgggagcatctcttgaggccaggagtttgagaccagcctgggcagcatagcaagacacatccctacaaaaaattagaaattggctggatgtggtggcatacgcctgtagtcctagccactcaggaggctgaggcaggaggattgcttgagcccaggagttcgaggctgcagtcagtcatgatggcaccactgcactccagcctgggcaacagagcaagatcctgtctttaaggaaaaaaagacaagatgagcataccagcagtccttgaacattatcaaaaagttcagcatattagaatcaccgggaggccttgttaaaagagttcgctgggcccatcttcagagtctctgagttgttggtctggaatagagccaaatgttttgtgtgtctaacaattcccaggtgctgttgctgctgctactattccaggaacacactttgagaaccattgtgttattgctctgcacgcccacccactctcaactcccacgaaaaaaatcaacttccagagctaagatttcggtggaagtcctggttccatatctggtgcaagatctcccctcacgaatcagttgagtcaacattctagctcaacaacatcacacgattaacattaacgaaaattattcatttgggaaactatcagccagttttcacttctgaaggggcaggagagtgttatgagaaatcacggcagttttcagcagggtccagattcagattaaataactattttctgtcatttctgtgaccaaccacatacaaacagactcatctgtgcactctccccctcccccttcaggtatatgttttctgagtaaagttgaaaagaatctcagaccagaaaatatagatatatatttaaatcttacttgagtagaactgattacgacttttgggtgttgaggggtctataagatcaaaacttttccatgataatactaagatgttatcgaccatttatctgtccttctctcaaaagtgtatggtggaattttccagaagctatgtgatacgtgatgatgtcatcactctgctgttaacatataataaatttattgctattgtttataaaagaataaatgatatttttt

SEQ ID NO: 212 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 213 - DNA, 2740 bp, Homo sapiens
  source 1..2740: mol_type = unassigned DNA
aagaacgcccccaaaatctgtttctaattttacagaaatcttttgaaacttggcacggtattcaaaagtccgtggaaagaaaaaaaccttgtcctggcttcagcttccaactacaaagacagacttggtccttttcaacggttttcacagatccagtgacccacgctctgaagacagaattagctaactttcaaaaacatctggaaaaatgaagacttgggtaaaaatcgtatttggagttgccacctctgctgtgcttgccttattggtgatgtgcattgtcttacgcccttcaagagttcataactctgaagaaaatacaatgagagcactcacactgaaggatattttaaatggaacattttcttataaaacattttttccaaactggatttcaggacaagaatatcttcatcaatctgcagataacaatatagtactttataatattgaaacaggacaatcatataccattttgagtaatagaaccatgctttggagatactcttacacagcaacatattacatctatgaccttagcaatggagaatttgtaagaggaaatgagcttcctcgtccaattcagtatttatgctggtcgcctgttgggagtaaattagcatatgtctatcaaaacaatatctatttgaaacaaagaccaggagatccaccttttcaaataacatttaatggaagagaaaataaaatatttaatggaatcccagactgggtttatgaagaggaaatgcttgctacaaaatatgctctctggtggtctcctaatggaaaatttttggcatatgcggaatttaatgatacggatataccagttattgcctattcctattatggcgatgaacaatatcctagaacaataaatattccatacccaaaggctggagctaagaatcccgttgttcggatatttattatcgataccacttaccctgcgtatgtaggtccccaggaagtgcctgttccagcaatgatagcctcaagtgattattatttcagttggctcacgtgggttactgatgaacgagtatgtttgcagtggctaaaaagagtccagaatgtttcggtcctgtctatatgtgacttcagggaagactggcagacatgggattgtccaaagacccaggagcatatagaagaaagcagaactggatgggctggtggattctttgtttcaacaccagttttcagctatgatgccatttcgtactacaaaatatttagtgacaaggatggctacaaacatattcactatatcaaagacactgtggaaaatgctattcaaattacaagtggcaagtgggaggccataaatatattcagagtaacacaggattcactgttttattctagcaatgaatttgaagaataccctggaagaagaaacatctacagaattagcattggaagctatcctccaagcaagaagtgtgttacttgccatctaaggaaagaaaggtgccaatattacacagcaagtttcagcgactacgccaagtactatgcacttgtctgctacggcccaggcatccccatttccacccttcatgatggacgcactgatcaagaaattaaaatcctggaagaaaacaaggaattggaaaatgctttgaaaaatatccagctgcctaaagaggaaattaagaaacttgaagtagatgaaattactttatggtacaagatgattcttcctcctcaatttgacagatcaaagaagtatcccttgctaattcaagtgtatggtggtccctgcagtcagagtgtaaggtctgtatttgctgttaattggatatcttatcttgcaagtaaggaagggatggtcattgccttggtggatggtcgaggaacagctttccaaggtgacaaactcctctatgcagtgtatcgaaagctgggtgtttatgaagttgaagaccagattacagctgtcagaaaattcatagaaatgggtttcattgatgaaaaaagaatagccatatggggctggtcctatggaggatacgtttcatcactggcccttgcatctggaactggtcttttcaaatgtggtatagcagtggctccagtctccagctgggaatattacgcgtctgtctacacagagagattcatgggtctcccaacaaaggatgataatcttgagcactataagaattcaactgtgatggcaagagcagaatatttcagaaatgtagactatcttctcatccacggaacagcagatgataatgtgcactttcaaaactcagcacagattgctaaagctctggttaatgcacaagtggatttccaggcaatgtggtactctgaccagaaccacggcttatccggcctgtccacgaaccacttatacacccacatgacccacttcctaaagcagtgtttctctttgtcagactaaaaacgatgcagatgcaagcctgtatcagaatctgaaaaccttatataaacccctcagacagtttgcttattttattttttatgttgtaaaatgctagtataaacaaacaaattaatgttgttctaaaggctgttaaaaaaaagatgaggactcagaagttcaagctaaatattgtttacattttctggtactctgtgaaagaagagaaaagggagtcatgcattttgctttggacacagtgttttatcacctgttcatttgaagaaaaataataaagtcagaagttcaagtgctaaaaaaaaaaaaaaaaaaaaaaaaaaaaaa

SEQ ID NO: 214 - protein, 735 aa, Homo sapiens
MKTWVKIVFGVATSAVLALLVMCIVLRPSRVHNSEENTMRALTLKDILNGTFSYKTFFPNWISGQEYLHQSADNNIVLYNIETGQSYTILSNRTMLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 215 - DNA, 3913 bp, Homo sapiens
  source 1..3913: mol_type = unassigned DNA
ctttcactggcaagagacggagtcctgggtttcagttccagttgcctgcggtgggctgtgtgagtttgccaaagtcccctgccctctctgggtctcggttccctcgcctgtccacgtgaggttggaggagctgaacgccgacgtcatttttagctaagagggagcagggtccccgagtcgccggcccagggtctgcgcatccgaggccgcgcgccctttcccctcccccacggctcctccgggccccgcactctgcgccccggctgccgcccagcgccctacaccgccctcagggggccctcgcgggctccccccggccgggatgccagtgccccgcgccacgcgcgcctgctcccgcgccgcctgccctgcagcctgcccgcggcgcctttatacccagcgggctcggcgctcactaatgtttaactcggggccgaaacttgccagcggcgagtgactccaccgcccggagcagcggtgcaggacgcgcgtctccgccgcccgcggtgacttctgcctgcgctccttctctgaacgctcacttccgaggagacgccgacgatgaagacaccgtggaaggttcttctgggactgctgggtgctgctgcgcttgtcaccatcatcaccgtgcccgtggttctgctgaacaaaggcacagatgatgctacagctgacagtcgcaaaacttacactctaactgattacttaaaaaatacttatagactgaagttatactccttaagatggatttcagatcatgaatatctctacaaacaagaaaataatatcttggtattcaatgctgaatatggaaacagctcagttttcttggagaacagtacatttgatgagtttggacattctatcaatgattattcaatatctcctgatgggcagtttattctcttagaatacaactacgtgaagcaatggaggcattcctacacagcttcatatgacatttatgatttaaataaaaggcagctgattacagaagagaggattccaaacaacacacagtgggtcacatggtcaccagtgggtcataaattggcatatgtttggaacaatgacatttatgttaaaattgaaccaaatttaccaagttacagaatcacatggacggggaaagaagatataatatataatggaataactgactgggtttatgaagaggaagtcttcagtgcctactctgctctgtggtggtctccaaacggcacttttttagcatatgcccaatttaacgacacagaagtcccacttattgaatactccttctactctgatgagtcactgcagtacccaaagactgtacgggttccatatccaaaggcaggagctgtgaatccaactgtaaagttctttgttgtaaatacagactctctcagctcagtcaccaatgcaacttccatacaaatcactgctcctgcttctatgttgataggggatcactacttgtgtgatgtgacatgggcaacacaagaaagaatttctttgcagtggctcaggaggattcagaactattcggtcatggatatttgtgactatgatgaatccagtggaagatggaactgcttagtggcacggcaacacattgaaatgagtactactggctgggttggaagatttaggccttcagaacctcattttacccttgatggtaatagcttctacaagatcatcagcaatgaagaaggttacagacacatttgctatttccaaatagataaaaaagactgcacatttattacaaaaggcacctgggaagtcatcgggatagaagctctaaccagtgattatctatactacattagtaatgaatataaaggaatgccaggaggaaggaatctttataaaatccaacttagtgactatacaaaagtgacatgcctcagttgtgagctgaatccggaaaggtgtcagtactattctgtgtcattcagtaaagaggcgaagtattatcagctgagatgttccggtcctggtctgcccctctatactctacacagcagcgtgaatgataaagggctgagagtcctggaagacaattcagctttggataaaatgctgcagaatgtccagatgccctccaaaaaactggacttcattattttgaatgaaacaaaattttggtatcagatgatcttgcctcctcattttgataaatccaagaaatatcctctactattagatgtgtatgcaggcccatgtagtcaaaaagcagacactgtcttcagactgaactgggccacttaccttgcaagcacagaaaacattatagtagctagctttgatggcagaggaagtggttaccaaggagataagatcatgcatgcaatcaacagaagactgggaacatttgaagttgaagatcaaattgaagcagccagacaattttcaaaaatgggatttgtggacaacaaacgaattgcaatttggggctggtcatatggagggtacgtaacctcaatggtcctgggatcgggaagtggcgtgttcaagtgtggaatagccgtggcgcctgtatcccggtgggagtactatgactcagtgtacacagaacgttacatgggtctcccaactccagaagacaaccttgaccattacagaaattcaacagtcatgagcagagctgaaaattttaaacaagttgagtacctccttattcatggaacagcagatgataacgttcactttcagcagtcagctcagatctccaaagccctggtcgatgttggagtggatttccaggcaatgtggtatactgatgaagaccatggaatagctagcagcacagcacaccaacatatatatacccacatgagccacttcataaaacaatgtttctctttaccttagcacctcaaaataccatgccatttaaagcttattaaaactcatttttgttttcattatctcaaaactgcactgtcaagatgatgatgatctttaaaatacacactcaaatcaagaaacttaaggttacctttgttcccaaatttcatacctatcatcttaagtagggacttctgtcttcacaacagattattaccttacagaagtttgaattatccggtcgggttttattgtttaaaatcatttctgcatcagctgctgaaacaacaaataggaattgtttttatggaggctttgcatagattccctgagcaggattttaatctttttctaactggactggttcaaatgttgttctcttctttaaagggatggcaagatgtgggcagtgatgtcactagggcagggacaggataagagggattagggagagaagatagcagggcatggctgggaacccaagtccaagcataccaacacgagcaggctactgtcagctcccctcggagaagagctgttcacagccagactggcacagttttctgagaaagactattcaaacagtctcaggaaatcaaatatgcaaagcactgacttctaagtaaaaccacagcagttgaaaagactccaaagaaatgtaagggaaactgccagcaacgcaggcccccaggtgccagttatggctataggtgctacaaaaacacagcaagggtgatgggaaagcattgtaaatgtgcttttaaaaaaaaatactgatgttcctagtgaaagaggcagcttgaaactgagatgtgaacacatcagcttgccctgttaaaagatgaaaatatttgtatcacaaatcttaacttgaaggagtccttgcatcaatttttcttatttcatttctttgagtgtcttaattaaaagaatattttaacttccttggactcattttaaaaaatggaacataaaatacaatgttatgtattattattcccattctacatactatggaatttctcccagtcatttaataaatgtgccttcattttttcagaaaaaaaaaaaaaaa

SEQ ID NO: 216 - protein, 766 aa, Homo sapiens
MKTPWKVLLGLLGAAALVTIITVPVVLLNKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 217 - DNA, 2924 bp, Homo sapiens
  source 1..2924: mol_type = unassigned DNA
gacgccgacgatgaagacaccgtggaaggttcttctgggactgctgggtgctgctgcgcttgtcaccatcatcaccgtgcccgtggttctgctgaacaaaggcacagatgatgctacagctgacagtcgcaaaacttacactctaactgattacttaaaaaatacttatagactgaagttatactccttaagatggatttcagatcatgaatatctctacaaacaagaaaataatatcttggtattcaatgctgaatatggaaacagctcagttttcttggagaacagtacatttgatgagtttggacattctatcaatgattattcaatatctcctgatgggcagtttattctcttagaatacaactacgtgaagcaatggaggcattcctacacagcttcatatgacatttatgatttaaataaaaggcagctgattacagaagagaggattccaaacaacacacagtgggtcacatggtcaccagtgggtcataaattggcatatgtttggaacaatgacatttatgttaaaattgaaccaaatttaccaagttacagaatcacatggacggggaaagaagatataatatataatggaataactgactgggtttatgaagaggaagtcttcagtgcctactctgctctgtggtggtctccaaacggcacttttttagcatatgcccaatttaacgacacagaagtcccacttattgaatactccttctactctgatgagtcactgcagtacccaaagactgtacgggttccatatccaaaggcaggagctgtgaatccaactgtaaagttctttgttgtaaatacagactctctcagctcagtcaccaatgcaacttccatacaaatcactgctcctgcttctatgttgataggggatcactacttgtgtgatgtgacatgggcaacacaagaaagaatttctttgcagtggctcaggaggattcagaactattcggtcatggatatttgtgactatgatgaatccagtggaagatggaactgcttagtggcacggcaacacattgaaatgagtactactggctgggttggaagatttaggccttcagaacctcattttacccttgatggtaatagcttctacaagatcatcagcaatgaagaaggttacagacacatttgctatttccaaatagataaaaaagactgcacatttattacaaaaggcacctgggaagtcatcgggatagaagctctaaccagtgattatctatactacattagtaatgaatataaaggaatgccaggaggaaggaatctttataaaatccaacttagtgactatacaaaagtgacatgcctcagttgtgagctgaatccggaaaggtgtcagtactattctgtgtcattcagtaaagaggcgaagtattatcagctgagatgttccggtcctggtctgcccctctatactctacacagcagcgtgaatgataaagggctgagagtcctggaagacaattcagctttggataaaatgctgcagaatgtccagatgccctccaaaaaactggacttcattattttgaatgaaacaaaattttggtatcagatgatcttgcctcctcattttgataaatccaagaaatatcctctactattagatgtgtatgcaggcccatgtagtcaaaaagcagacactgtcttcagactgaactgggccacttaccttgcaagcacagaaaacattatagtagctagctttgatggcagaggaagtggttaccaaggagataagatcatgcatgcaatcaacagaagactgggaacatttgaagttgaagatcaaattgaagcagccagacaattttcaaaaatgggatttgtggacaacaaacgaattgcaatttggggctggtcatatggagggtacgtaacctcaatggtcctgggatcaggaagtggcgtgttcaagtgtggaatagccgtggcgcctgtatcccggtgggagtactatgactcagtgtacacagaacgttacatgggtctcccaactccagaagacaaccttgaccattacagaaattcaacagtcatgagcagagctgaaaattttaaacaagttgagtacctccttattcatggaacagcagatgataacgttcactttcagcagtcagctcagatctccaaagccctggtcgatgttggagtggatttccaggcaatgtggtatactgatgaagaccatggaatagctagcagcacagcacaccaacatatatatacccacatgagccacttcataaaacaatgtttctctttaccttagcacctcaaaataccatgccatttaaagcttattaaaactcatttttgttttcattatctcaaaactgcactgtcaagatgatgatgatctttaaaatacacactcaaatcaagaaacttaaggttacctttgttcccaaatttcatacctatcatcttaagtagggacttctgtcttcacaacagattattaccttacagaagtttgaattatccggtcgggttttattgtttaaaatcatttctgcatcagctgctgaaacaacaaataggaattgtttttatggaggctttgcatagattccctgagcaggattttaatctttttctaactggactggttcaaatgttgttctcttctttaaagggatggcaagatgtgggcagtgatgtcactagggcagggacaggataagagggattagggagagaagatagcagggcatggctgggaacccaagtccaagcataccaacacgaccaggctactgtcagctcccctcggagaaaactgtgcagtctgcgtgtgaacagctcttctcctttagagcacaatggatctcgagggatcttccatacctaccagttctgcgcctcgaggccgcgactctaga

SEQ ID NO: 218 - protein, 766 aa, Homo sapiens; identical to SEQ ID NO: 216

SEQ ID NO: 219 - DNA, 1260 bp, Homo sapiens
  source 1..1260: mol_type = unassigned DNA
atgattggcacagatcctcgaacaattcttaaagatttattgccggaaacaatacctccacctgagttggatgatatgacactgtggcagattgttattaatatcctttcagaaccaccaaaaaggaaaaaaagaaaagatattaatacaattgaagatgctgtgaaattactgcaagagtgcaaaaaaattatagttctaactggagctggggtgtctgtttcatgtggaatacctgacttcaggtcaagggatggtatttatgctcgccttgctgtagacttcccagatcttccagatcctcaagcgatgtttgatattgaatatttcagaaaagatccaagaccattcttcaagtttgcaaaggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttcctttgcaacagcatcttgcctgatttgtaaatacaaagttgactgtgaagctgtacgaggagctctttttagtcaggtagttcctcgatgtcctaggtgcccagctgatgaaccgcttgctatcatgaaaccagagattgtgttttttggtgaaaatttaccagaacagtttcatagagccatgaagtatgacaaagatgaagttgacctcctcattgttattgggtcttccctcaaagtaagaccagtagcactaattccaagttccataccccatgaagtgcctcagatattaattaatagagaacctttgcctcatctgcattttgatgtagagcttcttggagactgtgatgtcataattaatgaattgtgtcataggttaggtggtgaatatgccaaactttgctgtaaccctgtaaagctttcagaaattactgaaaaacctccacgaacacaaaaagaattggcttatttgtcagagttgccacccacacctcttcatgtttcagaagactcaagttcaccagaaagaacttcaccaccagattcttcagtgattgtcacacttttagaccaagcagctaagagtaatgatgatttagatgtgtctgaatcaaaaggttgtatggaagaaaaaccacaggaagtacaaacttctaggaatgttgaaagtattgctgaacagatggaaaatccggatttgaagaatgttggttctagtactggggagaaaaatgaa

SEQ ID NO: 220 - protein, 420 aa, Homo sapiens
MIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGALFSQVVPRCPRCPADEPLAIMKPEIVFFGENLPEQFHRAMKYDKDEVDLLIVIGSSLKVRPVALIPSSIPHEVPQILINREPLPHLHFDVELLGDCDVIINELCHRLGGEYAKLCCNPVKLSEITEKPPRTQKELAYLSELPPTPLHVSEDSSSPERTSPPDSSVIVTLLDQAAKSNDDLDVSESKGCMEEKPQEVQTSRNVESIAEQMENPDLKNVGSSTGEKNE

SEQ ID NO: 221 - DNA, 7341 bp, Homo sapiens
  source 1..7341: mol_type = unassigned DNA
gcgcgaggccgtcgattcgctcgcggctccatcgcggcctggccggggggcggtgtctgctgcgccaggttcgctggccgcacgtcttcaggtcctcctgttcctgggaggcgggcgcggcaggactgggaggtggcggcagcgggcgaggactcgccgaggacggggctccggcccgggataaccaactctccttctctcttctttggtgcttccccaggcggcggcggcggcgcccgggagccggagccttcgcggcgtccacgtccctcccccgctgcaccccgccccggcgcgagaggagagcgcgagagccccagccgcgggcgggcgggcggcgaagatggcagaggcaccggcttccccggccccgctctctccgctcgaagtggagctggacccggagttcgagccccagagccgtccgcgatcctgtacgtggcccctgcaaaggccggagctccaagcgagccctgccaagccctcgggggagacggccgccgactccatgatccccgaggaggaggacgatgaagacgacgaggacggcgggggacgggccggctcggccatggcgatcggcggcggcggcgggagcggcacgctgggctccgggctgctccttgaggactcggcccgggtgctggcacccggagggcaagaccccgggtctgggccagccaccgcggcgggcgggctgagcgggggtacacaggcgctgctgcagcctcagcaaccgctgccaccgccgcagccgggggcggctgggggctccgggcagccgaggaaatgttcgtcgcggcggaacgcctggggaaacctgtcctacgcggacctgatcacccgcgccatcgagagctccccggacaaacggctcactctgtcccagatctacgagtggatggtgcgttgcgtgccctacttcaaggataagggcgacagcaacagctctgccggctggaagaactccatccggcacaacctgtcactgcatagtcgattcatgcgggtccagaatgagggaactggcaagagctcttggtggatcatcaaccctgatggggggaagagcggaaaagccccccggcggcgggctgtctccatggacaatagcaacaagtataccaagagccgtggccgcgcagccaagaagaaggcagccctgcagacagcccccgaatcagctgacgacagtccctcccagctctccaagtggcctggcagccccacgtcacgcagcagtgatgagctggatgcgtggacggacttccgttcacgcaccaattctaacgccagcacagtcagtggccgcctgtcgcccatcatggcaagcacagagttggatgaagtccaggacgatgatgcgcctctctcgcccatgctctacagcagctcagccagcctgtcaccttcagtaagcaagccgtgcacggtggaactgccacggctgactgatatggcaggcaccatgaatctgaatgatgggctgactgaaaacctcatggacgacctgctggataacatcacgctcccgccatcccagccatcgcccactgggggactcatgcagcggagctctagcttcccgtataccaccaagggctcgggcctgggctccccaaccagctcctttaacagcacggtgttcggaccttcatctctgaactccctacgccagtctcccatgcagaccatccaagagaacaagccagctaccttctcttccatgtcacactatggtaaccagacactccaggacctgctcacttcggactcacttagccacagcgatgtcatgatgacacagtcggaccccttgatgtctcaggccagcaccgctgtgtctgcccagaattcccgccggaacgtgatgcttcgcaatgatccgatgatgtcctttgctgcccagcctaaccagggaagtttggtcaatcagaacttgctccaccaccagcaccaaacccagggcgctcttggtggcagccgtgccttgtcgaattctgtcagcaacatgggcttgagtgagtccagcagccttgggtcagccaaacaccagcagcagtctcctgtcagccagtctatgcaaaccctctcggactctctctcaggctcctccttgtactcaactagtgcaaacctgcccgtcatgggccatgagaagttccccagcgacttggacctggacatgttcaatgggagcttggaatgtgacatggagtccattatccgtagtgaactcatggatgctgatgggttggattttaactttgattccctcatctccacacagaatgttgttggtttgaacgtggggaacttcactggtgctaagcaggcctcatctcagagctgggtgccaggctgaaggatcactgaggaaggggaagtgggcaaagcagaccctcaaactgacacaagacctacagagaaaaccctttgccaaatctgctctcagcaagtggacagtgataccgtttacagcttaacacctttgtgaatcccacgccattttcctaacccagcagagactgttaatggccccttaccctgggtgaagcacttacccttggaacagaactctaaaaagtatgcaaaatcttccttgtacagggtggtgagccgcctgccagtggaggacagcacccctcagcaccacccaccctcattcagagcacaccgtgagcccccgtcggccattctgtggtgttttaatattgcgatggtttatgggacgttttaagtgttgttcttgtgtttgttttcctttgactttctgagtttttcacatgcattaacttgcggtatttttctgttaaaatgttaaccgtccttcccctagcaaatttaaaaacagaaagaaaatgttgtaccagttaccattccgggttcgagcatcacaagcttttgagcgcatggaactccataaactaacaaattacataaactaaagggggattttctttcttcttttgtttggtagaaaattatccttttctaaaaactgaacaatggcacaattgtttgctatgtgcacccgtccaggacagaaccgtgcataggcaaaaggagtggagcacagcgtccggcccagtgtgtttccggttctgagtcagggtgatctgtggacgggaccccagcaccaagtctacgggtgccagatcagtagggcctgtgatttcctgtcagtgtcctcagctaatgtgaacagtgttggtctgctggttagaaactagaatattgatattttcaggaaagaaatcagctcagctctccactcattgccaaatgtcactaaagggtttagttttaaggagaaagaaaaggaaaaaaaaaaaaaacaaaaaagtcctgttttgctttgcagaacaaatgaacttacaggtgagcattaagcttgcagtgagaaatgtgcgaagagtaaaaacccaagtcaatgctgaggcagttctaacttcactgttttcctaaatacacatccttgattattttcagccttgctatataatctgatctgctagaagtgtatgagtgagaggcaatagcatacaaactgattttttaaatataagcttaggttgtaattgtacaagtgactcaatggaagtacaaaatagggcagttttaacttttttttctgcttctatggatttcattttgttgtgttttcaaaaagttatggtgctgtataggtgctttctgtttaacctggaaagtgtgattatattcgttaccttctttggtagacggaatagttgggaccacctttggtacataagaaattggtataacgatgctctgattagcacagtatatgcatacttctccaaagtgatatatgaagactcttttctttgcataaaaagcattaggcatataaatgtataaatatattttatcatgtacagtacaaaaatggaaccttatgcatgggccttaggaatacaggctagtatttcagcacagacttccctgcttgagttcttgctgatgcttgcaccgtgacagtgggcaccaacacagacgtgccacccaaccccctgcacacaccaccggccaccaggggcccccttgtgcgccttggctttataactcctctgggggtgatattggtggtgatcacagctcctagcataatgagagttccatttggtattgtcacacgtctcctgcctcgcttgggttgccatgtttgagcgatggccctgttgatttcaccctgccttttactgaatctgtaaattgttgtgcaattgtggttatagtagactgtagcacattgccttttctaaactgctacatgtttataatcttcatttttaaagtatgtgtaatttttttaagtatgtattctattcatatggtctgcttgtcagtgagccagacttgcttactatattcctttataataatgctagccacttcctggattctttagtaatgtgctgtatgcaagaactttccagtagcagtgaaggagggttgcctctccaagcttcctaagggatgctgccctgtgtggggatgcattgcagaggcactagtagcatgggggctagagtggggagcgagatgtaaaagggtggggggataggagaattccagagtgcttccagcattagggtcctgagaacttctgagttcagagaaacatgcaaagtgactaacaaaatagctacttacctttgcagttttacagaccctgggagctgctttgggagtgagaaaggcaaccctccaatgtgtttcaactttaaaatgttgaattcttttcagacatggtatctcatttattctccttttctagcgtttgttgaatttcaggcagaatgtcttacagaatgtcctagaaccagattatcatttaatctgaaacagctgaggaagggacagagaaggtacaagggcaaggcagcacaaaacagatcaggagaatgaagagggaatgctttggttttttgttttgttttgttttttctttttcaagtaactaaaacagcatctacatgtagagtgttgtggagagctgagaccagggtaaagtcaagtgcagcatcagtactgcgagacccaccagcccctggagagggtcagccgagaatctggtagtgaagcctgtctagggtcccggcaccctcaccctcagccacctgcagagaggccagggccccagagactagcctggttctgaagtgggcaggggtgctgccagagccctctgccccttatgttgagaccctgctttcaggacaggccagccgttggccaccatgtcacattctgagtgagtgtcacaggtccctaacaataattttctgatctggagcatatcagcagaatgcttagcctcaaggggcctggcagctgtaatgtttgatttatgatgagaactatccgaggccacccttggcctctaaataagctgctctagggagccgcctactttttgatgagaaattagaagagtacctaatgttgaaaacatgacatgcgctcttgggatctgctgttctctccagggctccagaacctgatacctgttaccaaagctaggaaagagctttatcacaagccttcactgtcctggcatgagaactggctgccaggctcagtgtaccccattaactgtgaatgaatctgagcttggtttcctttattgcttcctctgcaatatgattgctgaaacacattttaaaaattcagaagcttgtcactcctgttaatgggaggatcagtcacacatgtgtagtacaaggcggactttgtgtttgtttttggtgttaatttttagcattgtgtgtgttgcttccccaccctgaggagaggacaccatggcttactactcaggacaagtatgccccgctcagggtgtgatttcaggtggcttccaaacttgtacgcagtttaaagatggtggggacagactttgcctctacctagtgaaccccacttaaagaataaggagcatttgaatctcttggaaaaggccatgaagaataaagcagtcaaaaagaagtcctccatgttggtgccaaggacttgcgaggggaaataaaaatgttatccagcctgaccaacatggagaaaccccgtctccattaaaaatacaaaattagcctggcatggtggcgcatgcctgtaatcccagctactctggaggctgaggcaggagaatcgcttgaacccaggaggcggaggtcgcagtgagccgagatcatgccagtgcactccagcctgggtaacaagagtgaaactccgtgtcaaaaaaaaaaaaaaaatgttactcatcctctctgaaagcaaaaaggaaaccctaacagctctgaactctggttttatttttcttgctgtatttgggtgaacattgtatgattaggcataatgttaaaaaaaaaaatttttttttggtagaaatgcaatcaccagtaaagaggtacgaaaaagctagcctctctcagagaccggggaggcagagtactactagaggaagtgaagttctgatggaatcatgcctgtcaaatgaggtcttgaagcggatgcccaaataaaagagtatattttatctaaatcttaagtgggtaacattttatgcagtttaaatgaatggaatattttcctcttgtttagttgtatctgtttgtatttttctttgatgaatgattggtcatgaggcctcttgccacactccagaaatacgtgtgcggctgcttttaagaactatgtgtctggtcacttatttctctaaaattatctcattgcctggcaatcagtcttctcttgtatacttgtcctagcacattatgtacatgggaaatgtaaacaaatgtgaaggaggaccagaaaaattagttaatatttaaaaaaatgtattgtgcattttggcttcacatgtttaactttttttaagaaaaaagttgcatgaatggaaaaaaaaatctgtatacagtatctgtaaaaactatcttatctgtttcaattccttgctcatatcccatataatctagaactaaatatggtgtgtggccatatttaaacacctgagagtcaagcagttgagactttgatttgaagcacctcatccttctttcaatgcgaacactatcatatggcattcttactgaggattttgtctaaccatatgttgccatgaattaactctgccgcctttcttaaggatcaaaaccagtttgatttgggaatcttcccctttccaaatgaaatagagatgcagtacttaactttccttggtgtttgtagatattgccttgtgtattccacttaaaaccgtaatctagtttgtaaaagagatggtgacgcatgtaaataaagcatcagtgacactct

SEQ ID NO: 222 - protein, 673 aa, Homo sapiens
MAEAPASPAPLSPLEVELDPEFEPQSRPRSCTWPLQRPELQASPAKPSGETAADSMIPEEEDDEDDEDGGGRAGSAMAIGGGGGSGTLGSGLLLEDSARVLAPGGQDPGSGPATAAGGLSGGTQALLQPQQPLPPPQPGAAGGSGQPRKCSSRRNAWGNLSYADLITRAIESSPDKRLTLSQIYEWMVRCVPYFKDKGDSNSSAGWKNSIRHNLSLHSRFMRVQNEGTGKSSWWIINPDGGKSGKAPRRRAVSMDNSNKYTKSRGRAAKKKAALQTAPESADDSPSQLSKWPGSPTSRSSDELDAWTDFRSRTNSNASTVSGRLSPIMASTELDEVQDDDAPLSPMLYSSSASLSPSVSKPCTVELPRLTDMAGTMNLNDGLTENLMDDLLDNITLPPSQPSPTGGLMQRSSSFPYTTKGSGLGSPTSSFNSTVFGPSSLNSLRQSPMQTIQENKPATFSSMSHYGNQTLQDLLTSDSLSHSDVMMTQSDPLMSQASTAVSAQNSRRNVMLRNDPMMSFAAQPNQGSLVNQNLLHHQHQTQGALGGSRALSNSVSNMGLSESSSLGSAKHQQQSPVSQSMQTLSDSLSGSSLYSTSANLPVMGHEKFPSDLDLDMFNGSLECDMESIIRSELMDADGLDFNFDSLISTQNVVGLNVGNFTGAKQASSQSWVPG

SEQ ID NO: 223 - DNA, 22 bp, Homo sapiens
  source 1..22: mol_type = unassigned DNA
tggctcagttcagcaggaacag

SEQ ID NO: 224 - DNA, 86 bp, Homo sapiens
  source 1..86: mol_type = unassigned DNA
tgccagtctctaggtccctgagaccctttaacctgtgaggacatccagggtcacaggtgaggttcttgggagcctggcgtctggcc

SEQ ID NO: 225 - DNA, 110 bp, Homo sapiens
  source 1..110: mol_type = unassigned DNA
gtgttggggactcgcgcgctgggtccagtggttcttaacagttcaacagttctgtagcgcaattgtgaaatgtttaggaccactagacccggcgggcgcggcgacagcga

SEQ ID NO: 226 - DNA, 100 bp, Homo sapiens
  source 1..100: mol_type = unassigned DNA
tgtgtctctctctgtgtcctgccagtggttttaccctatggtaggttacgtcatgctgttctaccacagggtagaaccacggacaggataccggggcacc

SEQ ID NO: 227 - DNA, 78 bp, Homo sapiens
  source 1..78: mol_type = unassigned DNA
ctgaggagcagggcttagctgcttgtgagcagggtccacaccaagtcgtgttcacagtggctaagttccgccccccag

SEQ ID NO: 228 - DNA, 110 bp, Homo sapiens
  source 1..110: mol_type = unassigned DNA
agaagggctatcaggccagccttcagaggactccaaggaacattcaacgctgtcggtgagtttgggatttgaaaaaaccactgaccgttgactgtaccttggggtcctta

SEQ ID NO: 229 - DNA, 21 bp, synthetic construct
  misc_feature 1..21: note = synthetic construct
  source 1..21: mol_type = other DNA
aagcctgtagcccacgtcgta

SEQ ID NO: 230 - DNA, 24 bp, synthetic construct
  misc_feature 1..24: note = synthetic construct
  source 1..24: mol_type = other DNA
ggcaccactagttggttgtctttg

SEQ ID NO: 231 - DNA, 76 bp, Mus musculus
  source 1..76: mol_type = unassigned DNA
ccatggaacattcaacgctgtcggtgagtttgggattcaaaaacaaaaaaaccaccgaccgttgactgtaccttgg

SEQ ID NO: 232 - protein, 4 aa, synthetic construct
KRSR

SEQ ID NO: 233 - protein, 5 aa, synthetic construct
YIGSR

SEQ ID NO: 234 - protein, 5 aa, synthetic construct
IKVAV

SEQ ID NO: 235 - protein, 5 aa, synthetic construct
RGDSK

SEQ ID NO: 236 - protein, 5 aa, synthetic construct
KRSRK

SEQ ID NO: 237 - protein, 6 aa, synthetic construct
GRGDYK

(19) United States
(12) Patent Application Publication
(10) Pub. No.: US 2024/0156853 A1
(43) Pub. Date: May 16, 2024

(54) IN VITRO AND IN VIVO INTRACELLULAR DELIVERY OF SIRNA VIA SELF-ASSEMBLED NANOPIECES

(71) Applicant: Rhode Island Hospital, Providence, RI (US)

(72) Inventors: Qian CHEN, Barrington, RI (US); Yupeng CHEN, Sharon, MA (US); Hongchuan YU, Chestnut Hill, MA (US)

(21) Appl. No.: 18/316,693

(22) Filed: May 12, 2023

Related U.S. Application Data
(63) Continuation of application No. 17/041,399, filed on Sep. 24, 2020, now Pat. No. 11,701,379.
(60) Provisional application No. 62/648,233, filed on Mar. 26, 2018.

(51) Int. Cl. A61K 31/713; A61K 47/54; A61K 47/69; A61P 19/02; C12N 15/113

(52) U.S. Cl. CPC A61K 31/713; A61K 47/545; A61K 47/6929; A61K 47/6949; A61P 19/02; C12N 15/1136; C12N 2310/141; C12N 2320/31

(57) ABSTRACT

The compositions and methods of the invention provide compositions and methods for preferential targeting of tissues to delivery therapeutic or diagnostic agents. For example, such compounds are useful in the treatment of joint disorders those affecting articulating joints, e.g., injury-induced osteoarthritis as well as autoimmune diseases affecting joint tissue such as rheumatoid arthritis.

### RELATED APPLICATIONS

This application is a continuation of U.S. patent application Ser. No. 17/041,399 filed Sep. 24, 2020, now U.S. Pat. No. 11,701,379, which is a national stage application filed under 35 U.S.C. § 371 of International Patent Application No. PCT/US2019/024155 filed Mar. 26, 2019, which claims the benefit of and priority under 35 U.S.C. § 119(e) to U.S. Provisional Application No. 62/648,233, filed Mar. 26, 2018, the entire contents of which are incorporated herein by reference in their entirety.

### STATEMENT OF GOVERNMENT INTERESTS

This invention was made with government support under P20 RR024484 and P20 GM104937 awarded by the National Institutes of Health. The Government has certain rights in the invention.

### INCORPORATION BY REFERENCE OF SEQUENCE LISTING

The contents of the sequence listing xml file named “405002-640001US_SL.xml”, which was created on Oct. 27, 2023 and is 405,118 bytes in size, is hereby incorporated by reference in its entirety.

### FIELD OF THE INVENTION

This invention relates to nanoparticles for delivering agents into cells or bodily tissues.

### BACKGROUND

Although progress in drug delivery using nanotechnology has been documented, several challenges remain, particularly with regard to targeting and toxicity. Current delivery systems suffer from significant hindrances such as low targeting efficiency.

### SUMMARY OF THE INVENTION

The compositions and methods of the invention provide a solution to long standing challenges in selective delivery of agents using nanotechnology. Accordingly, the invention features compounds, assemblies of such compounds, a system, or method for selective drug delivery to a cell or any bodily tissue (including those that include extracellular matrix tissue) comprising a nanoparticle. Nanoparticles such as rosette nanopieces, lipid nanoparticles, and polymeric nanoparticles composition comprise a cargo compound, wherein a positively-charged nanoparticle and cargo complex composition with net positive charge at pH 7-7.5 localizes or penetrates a negatively-charged tissue or wherein a negatively-charged (or weakly positively-charged) nanoparticle and cargo complex composition with net negative (or weak positive) charge at pH 7-7.5 localizes to or penetrates a positively-charged cell or tissue. “Negatively charged” means zeta-potential of equal or smaller than 0 mV (which is minus “−” mV). “Positively charged” means zeta-potential of equal or larger than 0 mV (which is plus “+” mV). “Weakly positive” means zeta potential of 0 mV to +30 mV. The nanoparticle is tuned to preferentially localize to and deliver its cargo (e.g., a drug) to a target bodily tissue. For example, a relatively negatively charged nanoparticle is used to preferentially localize to, accumulate, and/or penetrate a positively-charged tissue; a relatively positively charged nanoparticle is used to preferentially localize to, accumulate, and/or penetrate a negatively-charged tissue. For example, localization of the cargo-containing nanopiece is at least 10%, 20%, 50%, 75%, 2-fold, 5-fold, 8-fold, 10-fold or more to a target tissue compared to the level of localization/delivery of the cargo in the absence of the nanoparticle. Thus, the nanopieces are selectively localized to a desired bodily tissue and deliver the cargo there.

As described herein, the system for selective drug delivery to a cell or bodily tissue may include a cargo compound. In exemplary embodiments, the cargo compound is tumor necrosis factor-alpha (TNF-α) small interfering ribonucleic acid (siRNA); e.g., TNF-α siRNA. In other exemplary embodiments, the system for selective drug delivery to a cell includes delivery to a macrophage cell.

The drug or agent delivered comprises a diagnostic reagent or a therapeutic compound. In one example, a net positive charge comprises a Zeta potential in the range of +0 mV and +60 mV (e.g., 0.1 mV, 1, 5, 10, 20, 30, 45, 60 mV); exemplary negatively charged tissues include cartilage tissue or a chondrocyte cell. In another example, a charge comprising a Zeta potential in the range of −60 mV and +30 mV (e.g., −60, −50, −40, −30, −20, −10, 1, 10, 20, 30 mV) is used to selectively or preferentially target positively charged tissues; exemplary positively charged tissues include neuronal tissue or a neuron.

Also within the invention is a system for selective drug delivery to a bodily tissue comprising a nanoparticle composition comprising a cargo compound (e.g., a drug), the composition being sized to localize or penetrate a target tissue. The nanoparticle is at least 0.1 nm in at least one dimension. For example, a size of ≤150 nm (e.g., 0.1, 10, 25, 50, 75, 100, 125, 150 nm) in at least one dimension localizes to or penetrates synovium, ocular tissue, dermatologic tissue, mucosal tissue, or pulmonary tissue, a size of ≤100 nm (e.g., 0.1, 10, 25, 50, 75, 100 nm) in at least one dimension localizes to or penetrates kidney tissue, or a size of ≤30 nm (0.1, 2, 5, 10, 20, 25, 30 nm) in at least one dimension localizes to or penetrates heart tissue. A size of ≤90 nm (0.1, 2, 5, 10, 25, 50, 75, 80, 90 nm) in at least one dimension localizes to or penetrates cartilage with inflammation or defect, and a size of ≤30 nm (0.1, 2, 5, 10, 20, 25, 30 nm) in at least one dimension localizes to or penetrates healthy, intact cartilage.

The system or method includes the treatment of joint disorders those affecting articulating joints, e.g., injury-induced osteoarthritis as well as autoimmune diseases affecting joint tissue such as rheumatoid arthritis. The compositions and methods of the invention further provide a solution to long standing challenges in the treatment of diseases and/or disorders affecting the epithelial, connective, muscles and/or nervous tissues in the body. The invention provides methods of introducing a therapeutic or diagnostic agent into a cell or tissue or tissue matrix using rosette nanotubes or components of rosette nanotubes. Embodiments of the present disclosure include the formation of a composite or complex or combination of one or more agents, such as therapeutic or diagnostic agents, and a rosette nanotube or a component of a rosette nanotube, where the one or more agents are attached to or otherwise bound to the rosette nanotube or component of a rosette nanotube. Embodiments of the present disclosure are further directed to a product made by the process of mixing together rosette nanotubes as described herein or modules forming rosette nanotubes as described herein and one or more agents in aqueous media under conditions which cause the rosette nanotubes or components of rosette nanotubes to combine with the one or more agents to form a complex or combination in aqueous media where the one or more agents are attached or otherwise bound through steric, ionic, or other forces to the rosette nanotube a component of a rosette nanotube. According to one aspect, the one or more agents are bound by noncovalent forces.

The methods described herein can be used to treat a joint disease, and exemplary joint diseases include TNF-α mediated autoimmune diseases. TNF-α mediated autoimmune diseases include, for example, rheumatoid arthritis.

The nanopiece compositions are made from nanotubes made from modules that self-assemble, e.g., compounds comprising Formula I (module I) or compounds comprising Formula II (module II). Nanotubes according to the present disclosure include compounds of Formula I, or a salt thereof, below:

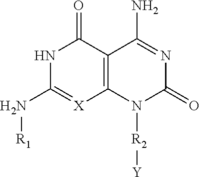

wherein X is CH or nitrogen; n is an integer of, 1, 2, 3, or 4; R₂ is hydrogen or a linker group for example (CH₂)ₙ or other linker groups described herein; Y is absent when R₂ is hydrogen or is an amino acid or polypeptide having an amino group covalently bound to an α-carbon of the amino acid and the amino group is covalently bound to the linker group R₂; and R₁ is hydrogen or an aliphatic moiety, such as alkyl, straight or branched chain, saturated or unsaturated; and salts thereof. Preferably R₁ is C₁ to C₁₀ alkyl, C₁ to C₅ alkyl, C₁ to C₃ alkyl, or methyl. For example, one subset of compounds of formula (I), or a salt thereof, includes those in which X is nitrogen. In another example, one subset of compounds of formula (I) includes those in which (CH₂)ₙ is the linker group. In another embodiment, one subset of compounds of formula (I) includes those in which (CH₂)ₙ is the linker group and n is 2. In another example, one subset of compounds of formula (I), or a salt thereof, includes those in which Y is an amino acid selected from lysine, arginine and histidine. In another embodiment, one subset of compounds of formula (I), or a salt thereof, includes those in which X is nitrogen, (CH₂)ₙ is the linker group, n is 2 and Y is an amino acid selected from lysine, arginine and histidine.

Compounds within the scope of the invention include those where the Y group can be connected to the linker group either by the amino group or the carboxyl group of the amino acid or polypeptide. An exemplary linker group is shown in the formula below.

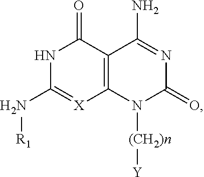

or a salt thereof.

An exemplary module within the scope of formula I is shown in FIG. 1 along with a schematic representation of a nanotube and an image of nanotubes formed from the exemplary module.

Alternative linker groups R₂ can join the Y group to the carbon of the (CH₂)ₙ group or the N atom either by the amino group or the carboxyl group of the amino acid or polypeptide.

Alternative R₂ groups within the scope of the present disclosure are selected from a group comprising:

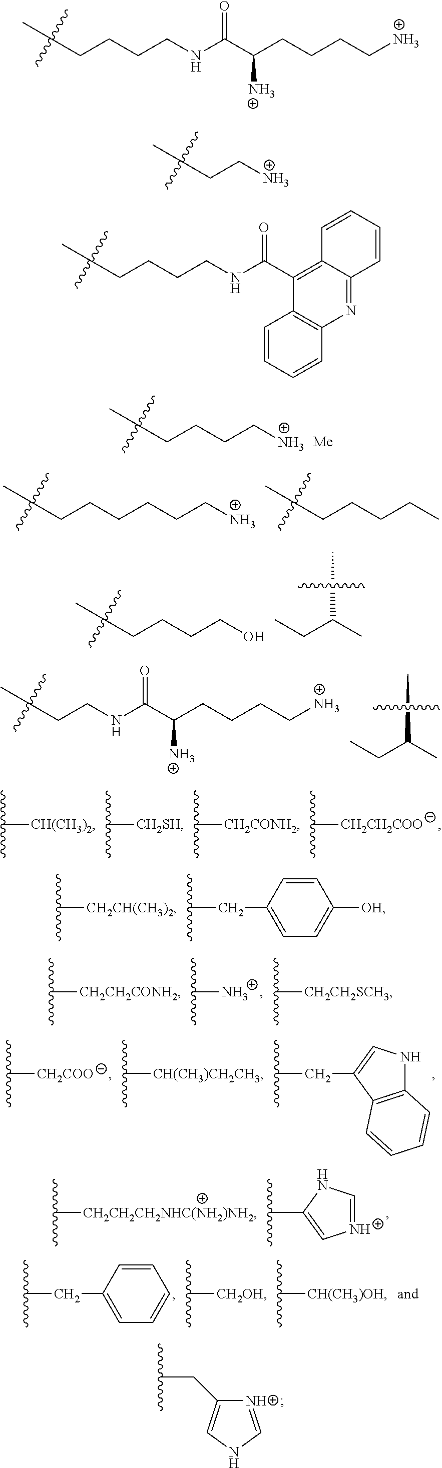

wherein Y is absent.
Compounds of Formula I can be prepared by the methods described in U.S. Pat. No. 6,696,565 hereby incorporated by reference herein in its entirety alone or combined with methods known to those of skill in the art. Additional description is provided in U.S. Pat. No. 8,795,691 and/or U.S. Patent Publication 20140171482 (U.S. Ser. No. 13/977,138), each of which is hereby incorporated by reference. Rosette nanotubes are made by assembly of compounds of Formula (I).

Exemplary compounds of Formula I are shown below:

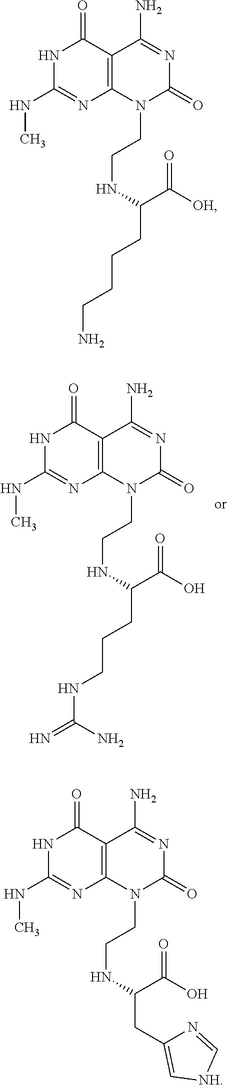

In certain embodiments, the compounds of Formula I are present in zwitterion form. The compounds are shown as below:

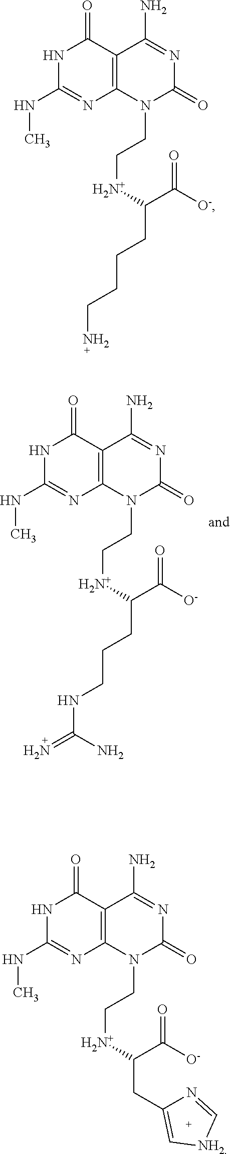

The salt forms thereof may be presented as follows:

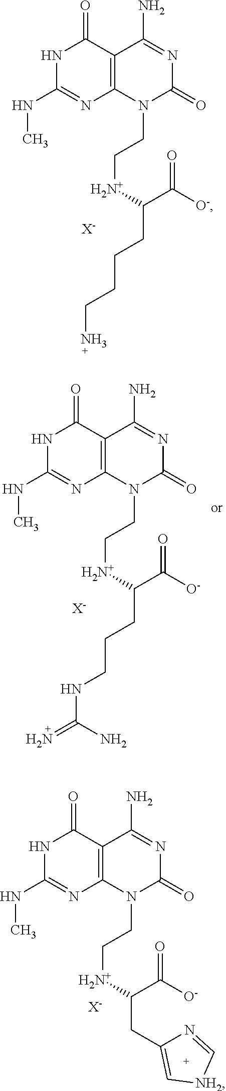

wherein X⁻ can be anion such as Cl⁻, or other anionic organic acids.

Modules according to the present disclosure also include compounds of Formula II below:

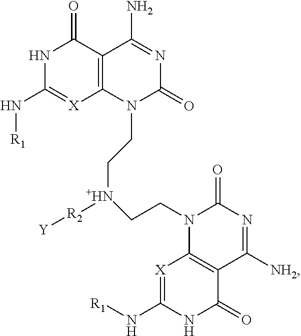

or a salt thereof.

wherein X is CH or nitrogen; R₂ is hydrogen or a linker group for example (CH₂)ₙ where n is an integer of, 1, 2, 3, or 4 or (CH₂)₃C₀ other linker groups described herein; Y is absent when R₂ is hydrogen or is an amino acid or polypeptide having an amino group covalently bound to an α-carbon of the amino acid and the amino group is covalently bound to the linker group R₂; and R₁ is hydrogen or an aliphatic moiety, such alkyl, straight or branched chain, saturated or unsaturated; and salts thereof. For example, one subset of compounds of formula (II) includes those in which X is nitrogen. In another example, one subset of compounds of formula (II) includes those in which (CH₂)ₙ is the linker group. In another embodiment, one subset of compounds of formula (II) includes those in which (CH₂)ₙ is the linker group and n is 2. In another example, one subset of compounds of formula (II) includes those in which Y is an amino acid selected from lysine, arginine and histidine. In another embodiment, one subset of compounds of formula (II) includes those in which X is nitrogen, (CH₂)ₙ is the linker group, n is 2 and Y is an amino acid selected from lysine, arginine and histidine.

Preferably R₁ is C₁ to C₁₀ alkyl, C₁ to C₅ alkyl, C₁ to C₃ alkyl, or methyl. An exemplary linker group is shown in the formula below.

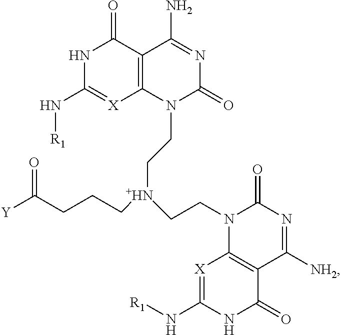

or a salt thereof.
Compounds within the scope of the present disclosure include those where the Y group can be connected to the linker group either by the amino group or the carboxyl group of the amino acid or polypeptide. Alternative R₂ groups within the scope of the present disclosure are selected from a group comprising:

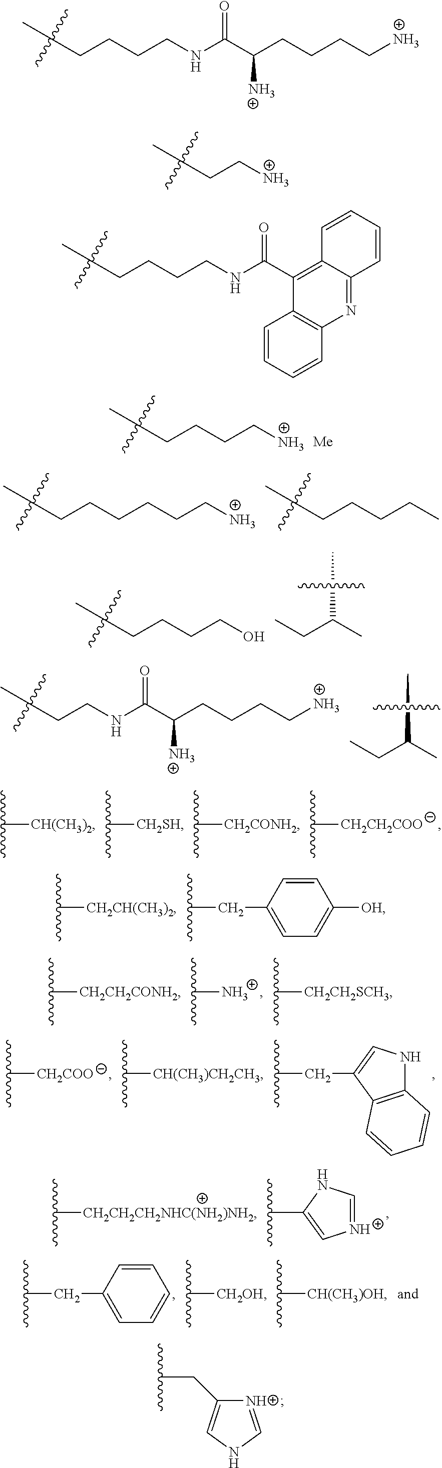

wherein Y is absent. TBL structures are made by the assembly of compounds of Formula (II).

Exemplary compounds of Formula II are shown below:

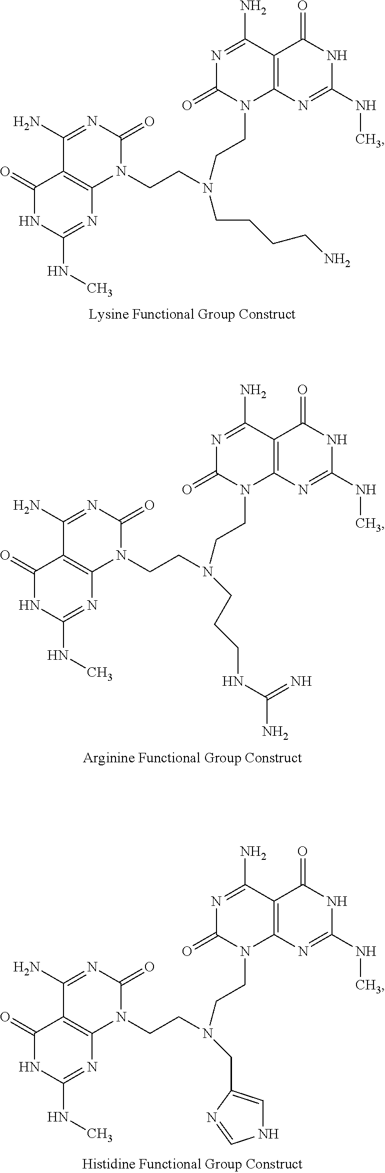 [SMILES: CNc1nc2c(c(N)nc(=O)n2CCN(CCCCN)CCn2c(=O)nc(N)c3c(=O)[nH]c(NC)nc32)c(=O)[nH]1 ; CNc1nc2c(c(N)nc(=O)n2CCN(CCCNC(=N)N)CCn2c(=O)nc(N)c3c(=O)[nH]c(NC)nc32)c(=O)[nH]1 ; CNc1nc2c(c(N)nc(=O)n2CCN(CCn2c(=O)nc(N)c3c(=O)[nH]c(NC)nc32)Cc2c[nH]cn2)c(=O)[nH]1]

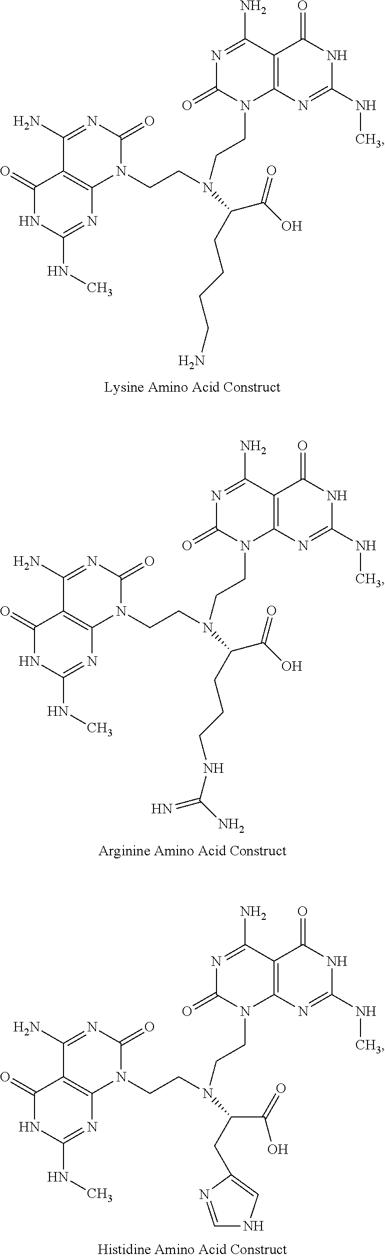 [SMILES: CNc1nc2c(c(N)nc(=O)n2CCN(CCn2c(=O)nc(N)c3c(=O)[nH]c(NC)nc32)[C@@H](CCCCN)C(=O)O)c(=O)[nH]1 ; CNc1nc2c(c(N)nc(=O)n2CCN(CCn2c(=O)nc(N)c3c(=O)[nH]c(NC)nc32)[C@@H](CCCNC(=N)N)C(=O)O)c(=O)[nH]1 ; CNc1nc2c(c(N)nc(=O)n2CCN(CCn2c(=O)nc(N)c3c(=O)[nH]c(NC)nc32)[C@@H](Cc2c[nH]cn2)C(=O)O)c(=O)[nH]1]

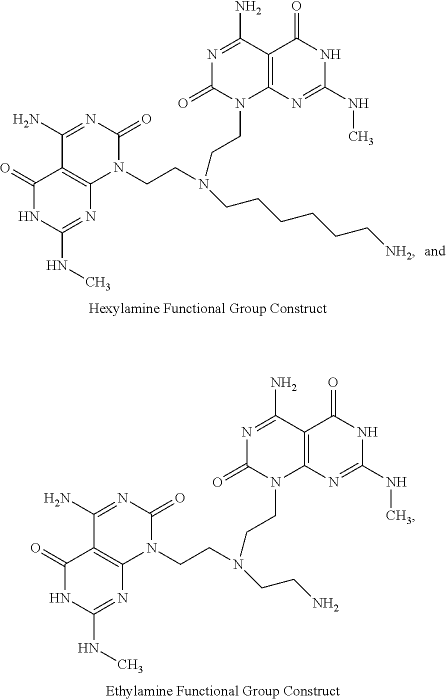 [SMILES: CNc1nc2c(c(N)nc(=O)n2CCN(CCCCCCN)CCn2c(=O)nc(N)c3c(=O)[nH]c(NC)nc32)c(=O)[nH]1 ; CNc1nc2c(c(N)nc(=O)n2CCN(CCN)CCn2c(=O)nc(N)c3c(=O)[nH]c(NC)nc32)c(=O)[nH]1]

or a salt thereof.

In some embodiments, compounds of formula II comprise amino acid functional group constructs. These compounds contain functional groups present in natural occurring amino acid side chains or may contain the entire amino acid side chain. For example, the lysine functional group constructs contains the entire amino acid side chain functionality (—CH₂CH₂CH₂CH₂NH₃⁺), whereas the histidine functional group constructs contains the entire side chain or only contains the heteroaryl imidazole group present in histidine.

In some embodiments, compounds of formula II comprise amino acid constructs. These compounds contain the entire the amino acid or may contain modified and/or unnatural amino acids. For example, the lysine amino acid analog contains the entire amino acid functionality of lysine, whereas the histidine amino acid constructs contains a modified histidine amino acid.

In some embodiments the compound of formula II is the Lysine Functional Group Construct

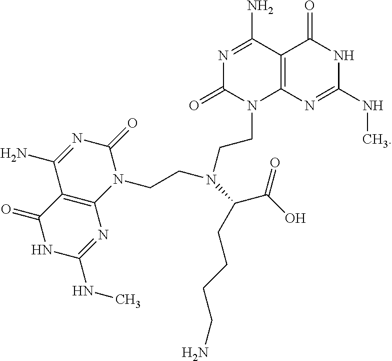 [SMILES: CNc1nc2c(c(N)nc(=O)n2CCN(CCn2c(=O)nc(N)c3c(=O)[nH]c(NC)nc32)[C@@H](CCCCN)C(=O)O)c(=O)[nH]1]

In some embodiments, the nanoparticles are constructed from lipid and/or polymeric components.

A three-dimensional representation of such modules is shown in FIG. 65. Embodiments further include delivering the composite into living cells. Embodiments further include a method of treating an individual requiring treatment comprising administering a complex of a rosette nanotube or a component of a rosette nanotube and one or more therapeutic agents to the individual in a manner to introduce the complex into cells or tissues of the individual. Embodiments further include a method of diagnosing an individual requiring diagnosis comprising administering a complex of a rosette nanotube or a component of a rosette nanotube and one or more diagnostic agents to the individual in a manner to introduce the complex into cells or tissues of the individual.

Rosette nanotubes or RNTs include nanotubes formed from modules having twin bases with a linker or TBL. Such rosette nanotubes may be referred to herein as “TBLs.” According to this aspect, the agent is delivered into the cell. According to one aspect, the agent is released from the rosette nanotube after entry into the cell. According to an additional aspect, the agent remains attached to, bound to, or complexed with or combined with the rosette nanotube or component of a rosette nanotube.

Lipid nanoparticles comprise a lipid core and surfactant, in which the lipid core may include fatty acids, acrylglycerols, steroids, waxes, and mixtures of all above; and surfactants may contain a positively charged amino group, negatively charged phosphate or carboxylic acid. According to one aspect, a complex is produced by combining modules of a self-assembled rosette nanotube and one or more agents in media where the modules self-assemble into a rosette nanotube or components of a rosette nanotube which incorporates the one or more agents to form a complex of a rosette nanotube or component of a rosette nanotube and the one or more agents. According to an additional aspect, a complex is produced by combining a self-assembled rosette nanotube and one or more agents in media whereupon the one or more agents are incorporated into the rosette nanotube to form a complex of a rosette nanotube and one or more agents. The complex may then be contacted to cells whereupon the complex enters the cells. Without wishing to be bound by scientific theory, it is believes that the complex may enter cells by endocytosis. According to certain embodiments, the cells may be transformed cells, recombinant cells, malignant cells, or cells from primary cell lines. The transfection method may be performed on cells in vitro or in vivo.

The modules may be any of those known to persons of ordinary skill in the art such as G\hat{ }C motifs and A\hat{ }T motifs, unmodified or modified to include moieties or side chains, which self-assemble into helical rosette nanotubes. According to one embodiment, modules are placed into an aqueous medium where they self-assemble into a substructure such as a ring structure, such as a rosette, and the ring structures then self-assemble by stacking one on top of another to form a tubular structure, commonly referred to as a nanotube. Such modules, substructures and nanometer scale molecular structures and their self-assembly is described in U.S. Pat. No. 6,696,565, Fenniri et al, J Am. Chem. Soc. 2001, 123, 3854-3855, Moralez et al., J Am. Chem. Soc., 2005, 127, 8307-8309, Fine et al., International Journal of Nanomedicine 2009:4 91-97; and Zhang et al., Biomaterials 2009; 30(7):1309-1320 each of which are hereby incorporated by reference in their entireties for all purposes.

Rosette nanotubes of the present disclosure are very stable in water and lack virus-related safety concerns and toxicity at amounts of about 1 μg/ml. See Int. J Nanomedicine, 2008, 3(3):373-383; Small. 2008, 4(6):817-823; and Am. J. Physiol Lung Cell Mol. Physiol. 2005, November, 289(5):L698-708 each of which are hereby incorporated by reference in their entireties.

According to one aspect of the present disclosure, methods are provided where the self-assembly of precursors or modules incorporates the agent into or otherwise complexes the agent with, the self-assembled rosette nanotube or components of the rosette nanotube. According to another aspect, fully assembled rosette nanotubes can be incubated with one or more or a plurality of agents and the one or more or plurality of agents can complex with the fully assembled rosette nanotube to form a composite. According to one further aspect, the one or more or plurality of agents are joined to or bound to the self-assembled rosette nanotube through steric, ionic, van der Waals, dispersion or other noncovalent interactions to form a rosette nanotube or component of a rosette nanotube and agent complex useful as a complex to be administered to an individual. In another aspect of the invention, the agents comprise a therapeutic agent such as nucleic acid, peptide or small molecule. In an aspect of the invention, the therapeutic agent comprises a nucleic acid, wherein the nucleic acid comprises siRNA. In other aspects, the nucleic acid comprises TNF-α siRNA. In another embodiment, the nucleic acid comprises the sequence comprising of: AAG CCT GTA GCC CAC GTC GTA (SEQ ID NO: 229) and GGC ACC ACT AGT TGG TTG TCT TTG-3′ (SEQ ID NO: 230). In a further aspect of the invention, the therapeutic agent comprises an IL-1 receptor antagonist. In yet a further aspect of the invention, the agent comprises a diagnostic agent such as a molecular probe or a molecular beacon. For example, the molecular beacon or probe comprises MMP-13 or ADAMTS-5.

According to certain aspects of the invention, a method for treating joint disease comprises administration of an effective amount of a composition containing a nanotube rosette-agent complex. Such a method of diagnosing joint disease comprises administration of an effective amount of a composition containing a nanotube rosette-agent complex. Another aspect of the invention comprises joint disease such as autoimmune, degenerative, inflammatory, infectious, cancerous, viral, fungal, injured, trauma, genetic, trauma, mechanical, nutritional or mal-alignment derived. Another embodiment of the invention describes joint disease comprising rheumatoid arthritis, osteoarthritis, juvenile onset of rheumatoid arthritis (JRA), psoriatic arthritis, reactive arthritis, septic arthritis, tendinitis, or herniation. Therapeutic agents are used to treat joint disease, e.g., such agents include analgesic agents, anti-inflammatory agents, immunosuppresive agents, antifungal agents, antibiotic agents, lubricants, anti-cancer agents, NMDA receptor antagonists, or antiviral agents.

According to certain aspects of the invention, a method for treating tissue and/or organ disease comprises administration of an effective amount of a composition containing a nanotube rosette-agent complex. Such a method of diagnosing tissue and/or organ disease comprises administration of an effective amount of a composition containing a nanotube rosette-agent complex. Another aspect of the invention comprises a tissue and/or organ disease such as autoimmune, degenerative, inflammatory, infectious, cancerous, viral, fungal, injured, trauma, genetic, trauma, mechanical, nutritional or mal-alignment derived. Another embodiment of the invention describes tissue and/or muscle disease comprising the eye, skin, brain, spine, intestine, kidney, liver, and stomach. Another aspect of the invention describes therapeutic agents to treat joint, tissue and/or organ disease, e.g., agents include analgesic agents, anti-inflammatory agents, immunosuppresive agents, antifungal agents, antibiotic agents, lurbicants, anti-cancer agents, NMDA receptor antagonists, or antiviral agents.

According to certain aspects, rosette nanotubes are functionalized with a nucleic acid, such as DNA or small RNA to form a complex, for example RNA is bound to the rosette nanotube, the complex is translocated into a cell or tissue, and the intracellular small RNA (e.g., siRNA) is present within the cell in an amount sufficient for gene silencing resulting in the inhibition of the production of target proteins. In this aspect, the rosette nanotube is a delivery vehicle or carrier for the small RNA into a cell for RNA interference purposes. Alternatively, the nucleic acid can be expressed by the cell. For example, the cell comprises synoviocytes or chondrocytes. Alternatively, the target tissue is cartilage. According to certain aspects, methods and technologies are provided to process and assemble rosette nanotubes (RNTs) for cargo delivery for diagnostic and therapeutic purpose. Methods are directed to achieve inter-/intra-cellular delivery in vitro and in vivo. According to certain aspects, a complex of rosette nanotubes (RNTs) and cargo agents are prepared. The cargo agents include diagnostic molecules, for instance, oligomer based molecular beacons; or therapeutic molecules such as nucleic acid, peptide, or small molecules. Such diagnostic agents and therapeutic agents are well known to those of skill in the art. Such incorporation between RNTs and the cargo reagent are facilitated by electrostatic force, π-π interactions or hydrophilic/hydrophobic effects to form relatively stable entities, which are referred to herein as “Nanopieces”. According to certain aspects, methods are provided to make rosette nanotubes of certain size (with or without an agent (e.g., cargo composition) that are suitable for trans-matrix e.g., extracellular matrix, tissue delivery. For example, methods are provided for altering at least one dimension or other parameter of Nanopieces such as width to infiltrate the pore size of the target tissue matrix. In an aspect of the invention, the rosette nanotubes are functionalzed with a nucleic acid (e.g., an siRNA), and in particular embodiments, TNF-α siRNA. In embodiments, the nucleic acid comprises the sequence comprising of: AAG CCT GTA GCC CAC GTC GTA (SEQ ID NO: 229) and GGC ACC ACT AGT TGG TTG TCT TTG-3′ (SEQ ID NO: 230).

In other examples, the methods for treating a joint disease described herein can include administering an effective amount of a rosette nanopiece, and the nanopiece can include a TNF-α siRNA. As provided herein, the nanopiece for trading a joint disease can enter the macrophage of a cell. In other exemplary embodiments, the nanopiece can be administered systemically.

According to certain aspects, methods and technologies are provided to process and assemble rosette nanotubes (RNTs) for cargo delivery for diagnostic and therapeutic purpose. Methods are directed to achieve inter-/intra-cellular delivery in vitro and in vivo. According to certain aspects, a complex of rosette nanotubes (RNTs) and cargo agents are prepared. The cargo agents include diagnostic molecules, for instance, oligomer based molecular beacons; or therapeutic molecules such as nucleic acid, peptide, or small molecules. In an aspect of the invention, the therapeutic agent comprises a nucleic acid, wherein the nucleic acid comprises siRNA. In other aspects, the nucleic acid comprises TNF-α siRNA. In another embodiment, the nucleic acid comprises the sequence comprising of: AAG CCT GTA GCC CAC GTC GTA (SEQ ID NO: 229) and GGC ACC ACT AGT TGG TTG TCT TTG-3′ (SEQ ID NO: 230). Such diagnostic agents and therapeutic agents are well known to those of skill in the art. Such incorporation between RNTs and the cargo reagent are facilitated by electrostatic force, π-π interactions or hydrophilic/hydrophobic effects to form relatively stable entities, which are referred to herein as “Nanopieces”. According to certain aspects, methods are provided to make rosette nanotubes of certain size with or without an agent that are suitable for trans-matrix tissue delivery. For example, methods are provided for altering at least one dimension parameter of Nanopieces such as width to infiltrate the pore size of the target tissue matrix.

According to certain aspects, methods are provided for making rosette nanotubes of certain lengths and size parameters such as 1) before assembly, controlling the length and bundle of RNTs via changing physical and/or chemical conditions such as temperature, molecule motion/vibration (like sonication) and pH; 2) during assembly, adjusting assembly conditions via changing physical and/or chemical conditions including concentrations, pH and ionic strength to enhance/reduce the formation and stacking of Nanopieces; 3) after assembly, breaking long or stacked Nanopieces by via changing physical and/or chemical conditions including enhancing molecule motion/vibration (like sonication).

According to certain aspects, methods are provided for trans-matrix/tissue delivery or a complex of a rosette nanotube or component or piece thereof by controlling the ratio between RNTs and cargo reagents so that the forming Nanopieces present surface charges that are suitable for attraction, localization, penetration, or retention in the tissue or one or more cells of the tissue. For example, since many tissues or cells contain negatively charged molecules (like proteoglycan), positively charged RNT can be fabricated and used to assemble with negatively charged nucleic acid cargo in certain ratios, resulting in a positive charged Nanopiece for delivery. In this manner, Nanopieces localize to, bind to, and accumulate onto/into the matrix/tissue resulting in much longer retention time to achieve more effective delivery. Therefore, the highly effective and versatile trans-matrix/tissue delivery was achieved by processed Nanopieces. The term “Nanopiece” may be used herein to refer to rosette nanotubes which may be processed into certain dimensions or components of rosette nanotubes.

According to certain aspects, methods are provided for the use of rosette nanotubes or Nanopieces for diagnostic applications insofar as molecular probes can be delivered via Nanopieces to detect a specific gene expression (or protein activity). By co-delivery of a negative control for non-specific signal and an internal positive control, a target gene expression can be accurately diagnosed in a real-time, in-situ and non-invasive manner.

According to certain aspects, therapeutic applications are envisioned, such as knocking down one or multiple disease gene expression (such as via siRNA, miRNA or anti-sense delivery), e.g., inhibiting the expression of one or more genes or gene products associated with aberrantly high expression in a disease state compared to a normal state up-regulating one or multiple beneficial gene/protein (such as via DNA, mRNA or protein delivery); or through a combination of both.

According to certain aspects, methods are provided for making rosette nanotubes of certain lengths and size parameters such as 1) before assembly, controlling the length and bundle of RNTs via changing physical and/or chemical conditions such as temperature, molecule motion/vibration (like sonication) and pH; 2) during assembly, adjusting assembly conditions via changing physical and/or chemical conditions including concentrations, pH and ionic strength to enhance/reduce the formation and stacking of Nanopieces; 3) after assembly, breaking long or stacked Nanopieces by via changing physical and/or chemical conditions including enhancing molecule motion/vibration (like sonication).

According to certain aspects, method are provided for trans-matrix/tissue delivery or a complex of a rosette nanotube or component or piece thereof by controlling the ratio between RNTs and cargo reagents so that the forming Nanopieces present surface charges that are suitable for retention in the tissue. For example, since many tissues or cells contain negatively charged molecules (like proteoglycan), positively charged RNT can be used to assemble with negatively charged nucleic acid cargo in certain ratios, resulting in a positive charged Nanopiece for delivery (see Table 1). In this manner, Nanopieces associate with, bind to and/or accumulate onto/into the matrix/tissue resulting in much longer retention time to achieve more effective delivery. Therefore, the highly effective and versatile trans-matrix/tissue delivery was achieved by processed Nanopieces.

According to certain aspects, methods are provided for the use of rosette nanotubes or Nanopieces for diagnostic applications insofar as molecular probes can be delivered via Nanopieces to detect a specific gene expression (or protein activity). By co-delivery of a negative control for non-specific signal and an internal positive control, a target gene expression can be accurately diagnosed in a real-time, in-situ and non-invasive manner.

According to certain aspects, therapeutic applications are envisioned, such as knocking down one or multiple disease gene expression (such as via siRNA delivery); up-regulating one or multiple beneficial gene/protein (such as via DNA, mRNA or protein delivery); or through a combination of both.

According to certain aspects, depending on the processing conditions, different sizes of rosette nanotubes, e. g. Nanopieces can be created for different delivery proposes, such as to enter a cellular or tissue matrix. For example, cartilage tissue matrix has about 60 nm mesh size of the collagen II fibrillar network (Comper et al in Cartilage: Molecular Aspects (eds Hall, B. & Newman, S.) 59-96 (CRC Press, Boston, 1991)) and about 20 nm spacing between the side chains of the proteoglycan network (Torzilli et al I Biomech. 30, 895-902 (1997)). Nanopieces with small sizes (at least one dimension smaller than 60 nm and/or 20 nm) showed excellent efficiency and function in intra-cartilage matrix delivery of siRNA. Secondly, through adjusting the ratio between RNTs and cargo reagents, overall positive charged surface enabled Nanopieces to adhere with negatively charged matrix/tissue components resulting longer retention time. Thirdly, Nanopieces can deliver a variety of cargo types and can deliver multiple cargo reagents at the same time. Fourthly, using non-covalent or covalent coating on Nanopieces can achieve a longer stability in the systemic circulation and penetrate into the targeted tissue matrix and/or organ more efficiently. Lastly, processed Nanopieces demonstrated successful delivery under conditions: in vitro, ex vivo and in vivo. Therefore, methods are provided for the use of Nanopieces for trans-matrix/tissue delivery.

According to certain aspects, complexes of rosette nanotubes or components of nanotubes or Nanopieces and agents can be used for research purposes as well as used for an effective delivery agent (especially in vivo) for molecular diagnosis and therapeutics. According to certain aspects, complexes of rosette nanotubes or components of nanotubes or Nanopieces and agents can be used for therapeutic purposes for treating various diseases, such as by delivery of interleukin-1 receptor antagonist (IL-1Ra), the natural protein inhibitor of IL-1, to modulate IL-1-based inflammation as a therapy for arthritis. For example, the cargo comprises IL-1R SiRNA. Complexes of rosette nanotubes or components of nanotubes or Nanopieces and agents can be used to deliver siRNA to knockdown the disease protein to achieve effective treatment.

According to certain aspects, complexes of rosette nanotubes or components of nanotubes or Nanopieces and agents can be used for diagnostics, such as by delivery of molecular probes or molecular beacons. Methods are provided to deliver molecular beacons into chondrocytes inside cartilage matrix as well as tissues and/or organs such as heart, stomach, kidney, liver, lung, spleen, brain, intestine, spine, rib cage, and limb. With co-delivery of multiple molecular beacons to detect disease gene expression as target, non-specific signal as negative control and house-keeping gene as internal positive control, target gene expression level can be quantified in a real-time, in-situ and non-invasive manner.

Embodiments of the present disclosure are directed to complexes of a self-assembled rosette nanotube and one or more or a plurality of agents. Such agents include biologically active agents and/or diagnostic agents. The complexes are administered to an individual where the biologically active agent and/or diagnostic agent are delivered to a site within the individual, including into the cell of an individual, and are made available for therapeutic or diagnostic purposes. According to one aspect, the agent dissociates from the rosette nanotube to treat an individual or to provide a diagnostic capability. According to an additional aspect, the agent remains attached to, bound to, or complexed with or combined with the rosette nanotube.

According to one aspect, a delivery complex is produced by combining modules of a self-assembled rosette nanotube and one or more agents, such as therapeutic or diagnostic agents, in media where the modules self-assemble into a rosette nanotube which incorporates the one or more agents to form a complex of a rosette nanotube and the one or more agents. According to an additional aspect, a delivery complex is produced by combining a self-assembled rosette nanotube and one or more agents, such as therapeutic or diagnostic agents, in media whereupon the one or more agents are incorporated into the rosette nanotube to form a complex of a rosette nanotube and one or more agents. The delivery complex may then be administered to an individual for therapeutic or diagnostic purposes. It is a further object of the present invention to create complexes of agents rosette nanotubes or components of rosette nanotubes that can be delivered into target cells and intracellular matrices where the agent can function. It is a further object of the present invention to provide methods of treating individuals using a delivery system of a complex of rosette nanotubes or components of rosette nanotubes and agents, where the agent enters the cell. These and other objects, features, and advantages of the invention or certain embodiments of the invention will be apparent to those skilled in the art from the following disclosure and description of exemplary embodiments.

It is a further object of the present invention to create complexes of agents rosette nanotubes or components of rosette nanotubes that can be delivered into target cells and intracellular matrices where the agent can function. It is a further object of the present invention to provide methods of treating individuals using a delivery system of a complex of rosette nanotubes or components of rosette nanotubes and agents, where the agent enters the cell. Thus, the invention encompasses a composition comprising a cargo molecule and a nanostructure comprising Formula I or Formula II for selective, e.g., preferential, delivery of a therapeutic drug or diagnostic agent to a target bodily tissue. Alternatively, the non-structure comprises a lipid or a polymer rather than a compound or Formula I or II.

In further examples, provided herein is a system for selective drug delivery to a bodily tissue. The system includes, for example a rosette nanopiece composition comprising a cargo compound. In further embodiments, the composition can sized to localize or penetrate a cell or target tissue. In other embodiments, a size of <100 nm in at least one dimension localizes or penetrates a phagocytic cell, synovium, ocular tissue, dermatologic tissue, mucosal tissue, or pulmonary tissue; a size of <90 nm in at least one dimension localizes or penetrates cartilage with inflammation or defect; a size of <50 nm in at least one dimension localizes or penetrates kidney tissue; a size of <30 nm in at least one dimension localizes or penetrates healthy, intact cartilage; or a size of <20 nm in at least one dimension localizes or penetrates heart tissue. In embodiments, as described herein, the phagocytic cell includes a macrophage.

Methods for treating a joint disease are also provided, wherein the method includes administering a composition comprising a nanopiece and a nucleic acid. In embodiments, the nanopiece includes a compound of Formula I or Formula II, or a salt thereof, or a combination thereof, and ii) a nucleic acid. In examples, the joint disease includes a TNF-α mediated autoimmune disease, e.g., rheumatoid arthritis. In other embodiments, the nanopiece for treating a joint disease enters a macrophage cell.

The methods and compositions for treating a joint disease include administering a composition including a nanopiece and a nucleic acid. In some examples, the nucleic acid comprises siRNA. In further embodiments, the siRNA is TNF-α siRNA, and the nucleic acid sequence of the TNF-α siRNA is AAG CCT GTA GCC CAC GTC GTA (SEQ ID NO: 229) and GGC ACC ACT AGT TGG TTG TCT TTG (SEQ ID NO: 230). In other embodiments, the nucleic acid is TNF-α. The joint disease can include rheumatoid arthritis or a cancer in the joint. The cancer can include sarcoma, hemangiopericytoma, connective tissue neoplasm, chondroma, or chondrosarcoma. In other examples, the nucleic acid is anti-miR-181a, and the anti-miR-181a nucleic acid sequence is SEQ ID NO: 228 or SEQ ID NO: 229.

Also provided and described herein are compositions for selective delivery of a therapeutic drug or diagnostic agent to a target cell or a bodily tissue. The composition can include a cargo molecule and a nanostructure comprising Formula I or Formula II, or a salt thereof. In other embodiments, the composition for selective drug delivery can further include a nucleic acid molecule (e.g., an siRNA). In exemplary embodiments, the siRNA is TNF-α siRNA, e.g., with the sequence comprising of: AAG CCT GTA GCC CAC GTC GTA (SEQ ID NO: 229) and GGC ACC ACT AGT TGG TTG TCT TTG (SEQ ID NO: 230). In other embodiments, the compositions can target a cell, e.g., a phagocytic cell. In further examples, the phagocytic cell includes a macrophage.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

FIG. 1 is an illustration showing an assembly between RNTs with siRNA.

FIG. 2 is an illustration showing an assembly between RNTs with plasmid DNA.

FIG. 3 is an illustration showing an assembly between RNTs with Matrilin-3.

FIG. 4 illustrates scheme 1, which displays an assembly mechanism and processing approaches.

FIG. 5A is a bar graph of the size distribution of Nanopieces assembled under standard conditions.

FIG. 5B is a bar graph of the width distribution of Nanopieces assembled under standard conditions.

FIG. 6A is a bar a graph of the size distribution of Nanopieces processed before assembly (quench).

FIG. 6B is a bar graph of the width distribution of Nanopieces processed before assembly (quench).

FIG. 7A is a bar graph of the size distribution of Nanopieces processed before assembly (sonication).

FIG. 7B is a bar graph of the width distribution of Nanopieces processed before assembly (sonication).

FIG. 8A is a bar graph of the size distribution of Nanopieces processed during assembly (increasing ionic strength).

FIG. 8B is a bar graph of the width distribution of Nanopieces processed during assembly (increasing ionic strength).

FIG. 9A is a bar graph of the size distribution of Nanopieces processed after assembly (increasing sonication time).

FIG. 9B is a bar graph of the width distribution of Nanopieces processed after assembly (increasing sonication time).

FIG. 10 is a series of images showing Nanopieces assembled before processing (Left) and after processing with sonication (Right) were delivered into cells.

FIG. 11 is a graph showing the Zeta potential (reflecting surface charge) of Nanopieces with different RNT/siRNA ratios.

FIG. 12 shows a series of images and a bar graph illustrating cartilage binding with RNTs, fluorescence labeled siRNA and RNT/siRNA Nanopieces on articular cartilage.

FIG. 13 is a series of images showing fluorescence labeled siRNA/RNT Nanopieces were delivered into porcine cartilage (Right) compared with controls (siRNA only).

FIG. 14 is a series of images showing effective delivery of processed GAPDH molecular beacon/RNT Nanopieces into mouse cartilage tissue matrix and inside chondrocytes.

FIG. 15 is a series of images showing effective delivery of processed GAPDH molecular beacon/RNT Nanopieces into human cartilage tissue matrix and inside chondrocytes.

FIG. 16 is a series of images showing effective delivery of processed GAPDH molecular beacon/RNT Nanopieces into chicken cartilage tissue matrix and inside chondrocytes.

FIG. 17 is a graph showing functional delivery of processed MATN3 siRNA/RNT Nanopieces into mouse cartilage tissue matrix and inside chondrocytes.

FIG. 18 is a graph showing functional delivery of processed MATN3 siRNA/RNT Nanopieces into mouse cartilage tissue matrix and inside chondrocytes.

FIG. 19 is a graph showing functional delivery of processed miRNA365/RNT Nanopieces into human cartilage tissue matrix and inside chondrocytes.

FIG. 20 is a graph showing functional delivery of processed miRNA365/RNT Nanopieces with and/or without PEG into human cartilage tissue matrix and inside chondrocytes in the serum and serum-free medium.

FIG. 21 is an image showing injection of reagents into mouse knee joints.

FIG. 22 is a series of images showing fluorescent signals in mouse cartilage tissue matrix over time by injecting processed RNT/beacon Nanopieces.

FIG. 23 is a series of images showing fluorescent signals in mouse cartilage tissue matrix over time by injecting molecular beacon only.

FIG. 24 is a graph showing quantitative fluorescent signals in mouse cartilage tissue matrix over time.

FIG. 25 is a graph and an image showing in vivo delivery of processed RNT/beacon Nanopieces into rat cartilage tissue matrix and inside chondrocytes compared with beacon only.

FIG. 26 is a series of images and a bar graph showing qualitative (Left) and quantitative (Right) in vivo delivery of processed RNT/beacon Nanopieces into rat cartilage tissue matrix and inside chondrocytes compared with beacon only.

FIG. 27 is an image showing injection of reagents into baby mouse joints.

FIG. 28 is a series of images showing histology sections of cartilage delivered with RNTs only (Top), beacon only (Middle) and RNT/beacon Nanopieces (Bottom).

FIG. 29 is a series of images showing in vitro validation of MMP-13 molecular beacon.

FIG. 30 is an image showing comparison of fluorescence signal between DMM and Sham knees (dark grey is GAPDH; light grey is MMP-13).

FIG. 31 is a graph showing DMM/Sham MMP-13 signal over time.

FIG. 32 is a graph showing DMM knee relative MMP-13 expression level.

FIG. 33 is a series of graphs showing relative IL-1R, MMP-13, MMP-9 and Col II gene expression level after therapeutically knock down of IL-1R.

FIG. 34 is a series of images showing histology (medium grey staining is proteoglycan) and immunohistochemistry (dark grey staining is epitope from aggrecan cleavage) of mouse knee joints. ADAMTS-5 siRNA/Nanopiece greatly inhibited cartilage degeneration and Aggrecan cleavage with cytokine stimulation.

FIG. 35 is a series of images showing histology of mouse knee joints. ADAMTS-5 siRNA/Nanopiece greatly inhibited cartilage degeneration after DMM surgery.

FIG. 36 is a graph showing histology evaluation of mouse knee joints. ADAMTS-5 siRNA/Nanopiece prevents osteoarthritis progression after DMM surgery.

FIG. 37 is a series of images showing a comparison with fluorescence signal from scrambled molecular beacon, signal from MMP-13 molecular beacon indicating the area of MMP-13 expression and articular cartilage degeneration.

FIG. 38 is an image of histology staining of a mouse knee joint after DMM surgery. The area of cartilage degeneration is the same as what was indicated by MMP-13 molecular beacon.

FIG. 39 is a series of images showing GAPDH and Scrambled molecular beacon delivered by Nanopieces into chondrocytes with stimulation.

FIG. 40 is a series of images showing GAPDH and ADAMTS-5 molecular beacon delivered by Nanopieces into chondrocytes without stimulation.

FIG. 41 is a series of images showing GAPDH and ADAMTS-5 molecular beacon was delivered by Nanopieces into chondrocytes with stimulation.

FIG. 42 is an image of fluorescence signal of ADAMTS-5 molecular beacon in DMM and Sham knees on day 6 after surgery.

FIG. 43 is a graph showing fluorescence signal ratio of ADAMTS-5 molecular beacon in DMM knees over Sham knees after surgery.

FIG. 44 is a series of images illustrating immunohistochemistry results (staining is epitope from aggrecan cleavage) of human articular cartilage. ADAMTS-4 siRNA and combination of ADAMTS-4&5 siRNA/Nanopieces greatly inhibited Aggrecan cleavage with cytokine stimulation.

FIG. 45 is a series of images showing histology results (staining is proteoglycan) of human articular cartilage. ADAMTS-4 siRNA and combination of ADAMTS-4&5 siRNA/Nanopieces greatly inhibited cartilage degradation with cytokine stimulation.

FIG. 46 is a series of images showing immunohistochemistry results (staining is epitope from aggrecan cleavage) of mouse knee joints. ADAMTS-5 siRNA/Nanopieces greatly inhibited Aggrecan cleavage after DMM surgery.

FIG. 47 is a graph showing cell toxicity studies of RNTs purified using HPLC chromatography with HCl or TFA as a modifier.

FIG. 48 is a series of images showing the conversion of nanotubes to nanorods.

FIG. 49 is a series of images showing the generation of Nanopieces before and after “processing −2”.

FIG. 50 is a graph showing quantitative analysis of fluorescence signal in mouse knee.

FIG. 51 is a scheme showing molecular beacon (MB) technology.

FIG. 52 is a scheme showing trans matrix delivery of Nanopieces into chondrocytes.

FIG. 53 is flow design of self-assembly, processing-1, processing-2 to yield nanopieces.

FIG. 54 is a graph showing MMP expression increase 4 days after surgery.

FIG. 55 is a graph showing MMP-expression increase 11 days after surgery.

FIG. 56 is a series of graphs an images showing Nanopieces size and morphology with increasing sonication power.

FIG. 57 is a scatter plot of Nanopieces size and morphology with increasing sonication power.

FIG. 58 is a line graph showing the stability of Nanopieces with different molar-excess ratios of PEG.

FIG. 59 is a line graph showing the stability of Nanopieces with and without non-covalent linked PEG.

FIG. 60 is an image showing the delivery of small Nanopieces into articular cartilage to result in fluorescence comparted to controls (MB only).

FIG. 61 is an image showing the delivery of both large and small Nanopieces into synovium to result in fluorescence compared with controls (MB only).

FIG. 62 is an image showing the decreased liver capture with small Nanopieces compared with lipid vehicles.

FIG. 63 is a bar graph showing the decreased liver capture with small Nanopieces compared to lipid vehicles.

FIG. 64 is a bar graph showing increased delivery into tissues or organs with dense matrix with small Nanopieces.

FIG. 65 is an illustration showing a structure of RNT. It is a long tubular structure with outside diameter of 3.5 nm, and inside diameter of 1.1 nm.

FIG. 66 is a series of images showing that cells with Nanopiece (RNT or TBL) delivery maintain normal cell morphology, indicating excellent biocompatibility of Nanopiece; while delivery with lipid-based vehicles led to abnormal cell morphology and large amount of debris, suggesting cyto-toxicity of lipid-based vehicles.

FIG. 67 is a bar graph showing PCR results of IL-1R expression levels of large and small lipid nanoparticles (*p<0.05 compared to negative controls and large lipid nanoparticle).

FIG. 68 is a bar graph showing PCR results of IL-1R expression levels of large and small polymer nanoparticles (*p<0.05 compared to negative controls and large polymer nanoparticle).

FIG. 69 shows amino acids containing hydrophilic side chains, hydrophobic side chains, and electrically charged side chains, respectively.

FIG. 70 is a schematic showing a strategy for RNA therapeutics delivery.

FIG. 71 is a schematic showing a Nanopiece™ (NP) delivery platform.

FIG. 72 is a schematic showing a NP^{JAK1/RNA} assembly and property.

FIG. 73 is a schematic showing a Nanopiece™ assembly and processing.

FIG. 74 is a schematic showing a Nanopiece™ tissue penetration.

FIG. 75 is a schematic showing a Nanopiece™ target delivery within cell.

FIG. 76 is a schematic showing targeted in vivo delivery.

FIG. 77 is a schematic showing problems and solutions associated with the clinical indication of post-traumatic joint injury (PTJI).

FIG. 78 is a schematic showing a therapeutic strategy for PTJI.

FIG. 79 is a schematic showing in vivo localized delivery for PTJI.

FIG. 80 is a schematic showing that a Nanopiece™ inhibits cartilage degeneration.

FIG. 81 is a brief description of chondrosarcoma and clinical needs.

FIG. 82 is a schematic showing in vivo data pertaining to inhibition of tumor growth and metastasis.

FIG. 83 is a brief description of rheumatoid arthritis (RA).

FIG. 84 is a schematic showing a therapeutic approach using siRNA and inhibition of TNF-α gene expression.

FIG. 85 is a series of bar graphs showing that TNF-α siRNA (SiTNF)/nanopiece (NP) treatment inhibits TNF-α mRNA levels in CIA mice.

FIG. 86 is a series of photographs showing that SiTNF/NP treatment inhibits bone erosions and destruction in CIA mice.

FIG. 87 is a description of siRNA administration using NPs.

FIG. 88 is a schematic showing nucleic acid delivery advantages using Nanopiece™.

FIG. 89A-C are images and results of the NP Rosette Nanotube. FIG. 89A is an image of the design of JBaK nanotube and formation of NP Rosette Nanotube. FIG. 89B is an image of the Nanopiece (side and top view). FIG. 89C are images of Nanopieces in TEM; TEM=Transmission electron microscopy.

FIG. 90A-90B are graphs showing the TNF-α expression. FIG. 90A are a series of graphs showing TNF-α mRNA expression of each group in PECs from the abdominal cavity (a—far left graph), knee joints (b—middle graph) and hind paws (c—far right graph) after twice injection of siRNA/NPs. In the mice with siTNF, the TNF-α mRNA expression was significantly knocked down. FIG. 90B is a graph showing the serum TNF-α levels of each group at 8 week. Serum TNF-α levels between siTNF and scrRNA mice were not significantly different. PECs: Peritoneal exudate cell macrophages, scrRNA: non-target siRNA, siTNF: TNF-α siRNA, CIA: collagen induced arthritis, *p<0.05, #P<0.005.

FIGS. 91A and 91B are graphs showing the total arthritis score and mechanical pain threshold. FIG. 91A is a graph showing the total arthritis score (n=6, each) of each treatment group of CIA mice with each treatment. *p<0.05, scrRNA: non-target siRNA, siTNF: TNF-α siRNA, CIA: collagen induced arthritis. FIG. 91B is a graph depicting the mechanical pain threshold of each treatment group of CIA mice with each treatment. *p<0.05, scrRNA: non-target siRNA, siTNF: TNF-α siRNA, CIA: collagen induced arthritis.

FIG. 92A-92D are a series of representative X-ray and 3D μCT images of CIA mice with each treatment. FIGS. 92A and 92 B are X-ray and 3D μCT images of the hind paw. FIGS. 92C and 92B are X-ray and 3D μCT images of the knee joint. In scRNA groups, there were multiple erosions at metatarsal joints and sever tarsal bone destruction. There were also many erosions on the surface of knee joints from mice with scrRNA treatment. CIA: collagen induced arthritis, siTNF: TNF-α siRNA, scrRNA: non-target siRNA

FIG. 93A-93C depict μCT images of subchondral bone from the tibia (FIG. 93A), femur (FIG. 93B), and calcaneus (FIG. 93). In each of the panels (a) represents subchondral bone, (b) represents the BV/TV, (c) represents BMD, (d) represents the trabecular number, (e) represents the trabecular thickness, both of which were (trabecular number and trabecular thickness) significantly higher in siTNF mice compared to scrRNA mice. The trabecular separation in siTNF mice was significantly narrower than scrRNA mice (panel f); n=5-7*P<0.005, #p=0.074. siTNF: TNF-α siRNA, scrRNA: non-target siRNA, BV: Bone Volume, TV: Total Volume, CIA: collagen induced arthritis.

FIG. 94A-94D represent a series of H&E and Safranin O staining images of CIA mice knee joints. H&E and Safranin O staining TNF-α siRNA/NP treatment inhibited synovium inflammation (FIG. 94A), cartilage degradation (FIG. 94B), bone loss (FIG. 94C), and meniscus destruction (FIG. 94D) in CIA mice. Representative histological images of knee joint of CIA mice. Scr siRNA: non-target siRNA, CIA: collagen induced arthritis, NPs: Nanopieces.

### DETAILED DESCRIPTION

The compositions and methods of the invention provide compositions and methods for preferential targeting of tissues to delivery therapeutic agents. The structures, e.g., nanopieces, are constructed to comprise a charge and/or size such that the structures preferentially associate with or bind to specific bodily tissues. For example, the invention provides methods for the delivery of Nanopieces and their cargo to target cells, tissues, or organs.

### Nanopieces for Treatment of Autoimmune Disease

TNF-α is a proinflammatory cytokine that is elevated in certain autoimmune diseases or disorders. Pathological deregulation characterizes those autoimmune diseases. TNF-α-mediated disease include rheumatoid arthritis, a debilitating autoimmune joint disease that is distinguished from osteoarthritis (which is not characterized as an autoimmune disease). Anti-TNF therapy has been used as a treatment for immune-inflammatory diseases, such as rheumatoid arthritis, juvenile RA, ankylosing spondylitis, psoriatic arthritis, Crohn's disease, ulcerative colitis, and psoriasis. These existing therapies use an anti-TNF-α antibody that neutralizes TNF-α function. In contrast, NP/TNF-α-siRNA described herein blocks the synthesis of TNF-α. The NP-mediated delivery system for TNF-α siRNA is associated with numerous important advantages including deep penetration into extracellular matrix-rich joint synovium and bone tissues that harbor macrophages, high efficiency in knocking down TNF-α synthesis, and low systemic toxicity.

In contrast to treatment for osteoarthritis, NP-mediated delivery for treatment of rheumatoid arthritis is carried out by systemic administration. Preferably, the treatment does not comprises local administration to a joint or joint space, e.g., does not comprise intra-articular administration. In contrast to treatment of osteoarthritis that targets joint cells such as chondrocytes, synovial cells, bone cells, joint capsule cells, and/or ligament cells, NP-mediated treatment for rheumatoid arthritis targets phagocytic cells such as macrophages, e.g., negatively-charged macrophages. Macrophages may be identified by one or more of the following markers: CD14, CD16, CD64, CD68, CD71, CCR5, CD11b, CD68, and/or CD163.

The rheumatoid arthritis (or other TNF-α-mediated autoimmune disease) therapy described herein targets and affects macrophages, i.e., the cells that produce TNF-α (which is the underlying defect of the pathology). The level of TNF-α production is therefore reduced or knocked down by the NP-mediated delivery of TNF-α siRNA to circulating macrophages or tissue-associated macrophages throughout the body. NP-mediated treatment for rheumatoid arthritis is preferably carried out by intravenous injection or infusion for treatment of numerous locations/tissues in the body that are affected by the disease.

The nanopieces described herein are positively charged. For example, the cargo, e.g., a nucleic acid such as a siRNA is negatively charged; however, when complexed with nanopieces (which are positively charged due to the presence of the positively-charged lysine of the nanopiece), the net charge of the complex is positive. The positively-charged NP-TNF-α siRNA complex associates with the negatively-charged macrophage cell and enters the cell, e.g., by endocytosis. In addition to entering cells by endocytosis, another advantage of the methods is when targeting macrophages, the cargo-loaded nanopieces may enter the cells by a second process, phagocytosis by macrophages—another advantage over previous approaches.

### NP-Based Delivery to Cargo to Cells or Bodily Tissue

A successful delivery into cells does not always necessarily mean that a successful delivery into tissue is achieved to obtain an efficacious therapeutic or diagnostic outcome. One major reason is that tissues unlike cells have an extracellular matrix. For example, Nanopieces with large size or inappropriate surface charge may not penetrate the tissue efficiently enough to cause a therapeutic or diagnostic response. Drug molecules released from nanotubes prior to tissue penetration do not diffuse into enough depth of the tissue to reach a significant amount of cells. The invention solves such problems and provides methods to package drug molecules within nanotubes/nanorods that are selectively designed to alter their surface charge and/or their size to be small enough to penetrate the tissue matrix. So in this manner it is not the drug molecules that are released from the nanotubes and then diffuse into the tissue but it is the actual Nanopieces/nanorods (containing cargo, e.g., drug) that penetrate the tissue. The invention further provides methods of processing nanotubes/nanorods to control of size and other properties of Nanopieces (like surface charge and coating), in order to efficiently deliver their cargo into joints, tissues and/or organs to achieve an effective therapy or diagnosis.

These Nanopieces (Nanopieces) may contain nucleic acid, peptides, proteins and aromatic or negatively charged small molecules. Because different tissues have different surface charge, it is important to control the surface charge of Nanopieces via the ratio of delivery cargos and amount of nanorods. Nanopieces, which are too large may have difficulties in penetrating the tissue matrix and improper surface charge of Nanopieces may be repulsive to the target tissue matrix or perhaps the Nanopieces are not stable in the bodily fluids or blood. The table below describes exemplary nanopieces for preferential localization to and delivery to exemplary bodily tissues.

### Selective Delivery of Nanopieces to Target Tissues

TABLE 1

| | | | Processing | | |
|---|---|---|---|---|---|
| | | Nanopiece | details to | | |
| Target | Nanopiece | Charge | achieve desired | Preferred | |
| Tissue/Cell | Structure | (Zeta | length/width/ | payload/ | Other/ |
| Type | Size | potential) | charge* | cargo | notes |
| Cartilage/ | General range: at | General | Ratio: 4.4~30 μg | siRNA, | Negatively |
| chondrocyte | least one dimension | range: | RNTs per | other | charged |
| | between 1 nm and 90 | between +0 | 0.1 nmol RNA | nucleic | |
| | nm | mV and | (Preferred ratio: | acids, | |
| | Preferred range: at | +60 mV | 6.6~20 μg RNTs | molecular | |
| | least one dimension | Preferred | per 0.1 nmol | beacons and | |
| | between 1 nm and 30 | range: | RNA) | peptides/ | |
| | nm | between +8 | Sonication | proteins | |
| | | mV and | power: | (ADAMTS- | |
| | | +40 mV | 10%~100% (for | 5 siRNA, | |
| | | | a 700 W | MMP-13 | |
| | | | sonicator) | oligo | |
| | | | Sonication time: | molecular | |
| | | | 10 s~30 mins | beacon, | |
| | | | Ionic strength of | IL-1Ra | |
| | | | assembly | protein) | |
| | | | solution: | | |
| | | | 0~308 mmol/L | | |
| | | | At least one of | | |
| | | | pre-processing | | |
| | | | methods (such | | |
| | | | as heating, | | |
| | | | sonication or | | |
| | | | quench): | | |
| | | | required | | |
| Synovium | General range: at | General | Ratio: 4.4~30 μg | siRNA, | |
| | least one dimension | range: | RNTs per | other | |
| | between 1 nm and | between +0 | 0.1 nmol RNA | nucleic | |
| | 150 nm | mV and | (Preferred ratio: | acids, | |
| | Preferred range: at | +60 mV | 4.4~20 μg RNTs | molecular | |
| | least one dimension | Preferred | per 0.1 nmol | beacons and | |
| | between 10 nm and | range: | RNA) | peptides/ | |
| | 100 nm | between +0 | Sonication | proteins | |
| | | mV and | power: 1~100% | (IL-1 or | |
| | | +40 mV | (for a 700 W | TNF-α | |
| | | | sonicator) | siRNA, | |
| | | | Sonication time: | IL-1 or | |
| | | | 5 s~30 mins | TNF-α oligo | |
| | | | Ionic strength of | molecular | |
| | | | assembly | beacon, | |
| | | | solution: no | IL-1Ra | |
| | | | requirement | protein) | |
| | | | At least one of | | |
| | | | pre-processing | | |
| | | | methods (such | | |
| | | | as heating, | | |
| | | | sonication or | | |
| | | | quench): not | | |
| | | | required | | |
| Neurons | General range: at | General | Ratio: 0.1~15 μg | siRNA, | Neurons |
| | least one dimension | range: | RNTs per | other | generally |
| | between 1 nm and | between | 0.1 nmol RNA | nucleic | posi- |
| | 150 nm | −60 mV and | (Preferred ratio: | acids, | tively |
| | Preferred range: at | +30 mV | 1~15 μg RNTs | molecular | charged |
| | least one dimension | Preferred | per 0.1 nmol | beacons and | |
| | between 10 nm and | range: | RNA) | peptides/ | |
| | 100 nm | between | Sonication | proteins | |
| | | −40 mV and | power: 1~100% | | |
| | | +30 mV | (for a 700 W | | |
| | | | sonicator) | | |
| | | | Sonication time: | | |
| | | | 5 s~30 mins | | |
| | | | Ionic strength of | | |
| | | | assembly | | |
| | | | solution: no | | |
| | | | requirement | | |
| | | | At least one of | | |
| | | | pre-processing | | |
| | | | methods (such | | |
| | | | as heating, | | |
| | | | sonication or | | |
| | | | quench): not | | |
| | | | required | | |
| Brain/BBB | General range: at | General | Ratio: 1~20 μg | siRNA, | |
| | least one dimension | range: | RNTs per | other | |
| | between 1 nm and | between | 0.1 nmol RNA | nucleic | |
| | 100 nm | 30 mV and | (Preferred ratio: | acids, | |
| | Preferred range: at | +40 mV | 4.4~20 μg RNTs | molecular | |
| | least one dimension | Preferred | per 0.1 nmol | beacons and | |
| | between 1 nm and 30 | range: | RNA) | peptides/ | |
| | nm | between +8 | Sonication | proteins | |
| | | mV and | power: | | |
| | | +40 mV | 10~100% (for a | | |
| | | | 700 W sonicator) | | |
| | | | Sonication time: | | |
| | | | 10 s~30 mins | | |
| | | | Ionic strength of | | |
| | | | assembly | | |
| | | | solution: | | |
| | | | 0~308 mmol/L | | |
| | | | At least one of | | |
| | | | pre-processing | | |
| | | | methods (such | | |
| | | | as heating, | | |
| | | | sonication or | | |
| | | | quench): | | |
| | | | required | | |
| Ocular | General range: at | General | Ratio: 4.4~30 μg | siRNA, | |
| tissue | least one dimension | range: | RNTs per | other | |
| | between 1 nm and | between +0 | 0.1 nmol RNA | nucleic | |
| | 150 nm | mV and | (Preferred ratio: | acids, | |
| | Preferred range: at | +60 mV | 4.4~20 μg RNTs | molecular | |
| | least one dimension | Preferred | per 0.1 nmol | beacons and | |
| | between 10 nm and | range: | RNA) | peptides/ | |
| | 100 nm | between +0 | Sonication | proteins | |
| | | mV and | power: 1~100% | | |
| | | +40 mV | (for a 700 W | | |
| | | | sonicator) | | |
| | | | Sonication time: | | |
| | | | 5 s~30 mins | | |
| | | | Ionic strength of | | |
| | | | assembly | | |
| | | | solution: no | | |
| | | | requirement | | |
| | | | At least one of | | |
| | | | pre-processing | | |
| | | | methods (such | | |
| | | | as heating, | | |
| | | | sonication or | | |
| | | | quench): not | | |
| | | | required | | |
| Derm tissue, | General range: at | General | Ratio: 4.4~30 μg | siRNA, | |
| skin, etc. | least one dimension | range: | RNTs per | other | |
| | between 1 nm and | between +0 | 0.1 nmol RNA | nucleic | |
| | 150 nm | mV and | (Preferred ratio: | acids, | |
| | Preferred range: at | +60 mV | 4.4~20 μg RNTs | molecular | |
| | least one dimension | Preferred | per 0.1 nmol | beacons and | |
| | between 10 nm and | range: | RNA) | peptides/ | |
| | 100 nm | between +0 | Sonication | proteins | |
| | | mV and | power: 1~100% | | |
| | | +40 mV | (for a 700 W | | |
| | | | sonicator) | | |
| | | | Sonication time: | | |
| | | | 5 s~30 mins | | |
| | | | Ionic strength of | | |
| | | | assembly | | |
| | | | solution: no | | |
| | | | requirement | | |
| | | | At least one of | | |
| | | | pre-processing | | |
| | | | methods (such | | |
| | | | as heating, | | |
| | | | sonication or | | |
| | | | quench): not | | |
| | | | required | | |
| Tumor | General range: at | General | Ratio: 0.1~30 μg | siRNA, | Tumors |
| | least one dimension | range: | RNTs per | other | may be |
| | between 1 nm and | between | 0.1 nmol RNA | nucleic | acidic |
| | 1200 nm | −60 mV and | (Preferred ratio: | acids, | |
| | Preferred range: at | +60 mV | 1~30 μg RNTs | molecular | |
| | least one dimension | Preferred | per 0.1 nmol | beacons and | |
| | between 10 nm and | range: | RNA) | peptides/ | |
| | 200 nm | between | Sonication | proteins | |
| | | −30 mV and | power: 1~100% | | |
| | | +60 mV | (for a 700 W | | |
| | | | sonicator) | | |
| | | | Sonication time: | | |
| | | | 5 s~30 mins | | |
| | | | Ionic strength of | | |
| | | | assembly | | |
| | | | solution: no | | |
| | | | requirement | | |
| | | | At least one of | | |
| | | | pre-processing | | |
| | | | methods (such | | |
| | | | as heating, | | |
| | | | sonication or | | |
| | | | quench): not | | |
| | | | required | | |
| Kidney | General range: at | General | Ratio: 4.4~30 μg | siRNA, | |
| | least one dimension | range: | RNTs per | other | |
| | between 1 nm and | between +0 | 0.1 nmol RNA | nucleic | |
| | 100 nm | mV and | (Preferred ratio: | acids, | |
| | Preferred range: at | +60 mV | 4.4~20 μg RNTs | molecular | |
| | least one dimension | Preferred | per 0.1 nmol | beacons and | |
| | between 10 nm and | range: | RNA) | peptides/ | |
| | 200 nm | between +0 | Sonication | proteins | |
| | | mV and | power: 5~100% | | |
| | | +40 mV | (for a 700 W | | |
| | | | sonicator) | | |
| | | | Sonication time: | | |
| | | | 5 s~30 mins | | |
| | | | Ionic strength of | | |
| | | | assembly | | |
| | | | solution: no | | |
| | | | requirement | | |
| | | | At least one of | | |
| | | | pre-processing | | |
| | | | methods (such | | |
| | | | as heating, | | |
| | | | sonication or | | |
| | | | quench): not | | |
| | | | required | | |
| Mucous | General range: at | General | Ratio: 4.4~30 μg | siRNA, | |
| membrane | least one dimension | range: | RNTs per | other | |
| | between 1 nm and | between +0 | 0.1 nmol RNA | nucleic | |
| | 150 nm | mV and | (Preferred ratio: | acids, | |
| | Preferred range: at | +60 mV | 4.4~20 μg RNTs | molecular | |
| | least one dimension | Preferred | per 0.1 nmol | beacons and | |
| | between 10 nm and | range: | RNA) | peptides/ | |
| | 100 nm | between +0 | Sonication | proteins | |
| | | mV and | power: 1~100% | | |
| | | +40 mV | (for a 700 W | | |
| | | | sonicator) | | |
| | | | Sonication time: | | |
| | | | 5 s~30 mins | | |
| | | | Ionic strength of | | |
| | | | assembly | | |
| | | | solution: no | | |
| | | | requirement | | |
| | | | At least one of | | |
| | | | pre-processing | | |
| | | | methods (such | | |
| | | | as heating, | | |
| | | | sonication or | | |
| | | | quench): not | | |
| | | | required | | |
| Lung | General range: at | General | Ratio: 4.4~30 μg | siRNA, | |
| | least one dimension | range: | RNTs per | other | |
| | between 10 nm and | between +0 | 0.1 nmol RNA | nucleic | |
| | 150 nm | mV and | (Preferred ratio: | acids, | |
| | Preferred range: at | +60 mV | 4.4~20 μg RNTs | molecular | |
| | least one dimension | Preferred | per 0.1 nmol | beacons and | |
| | between 20 nm and | range: | RNA) | peptides/ | |
| | 100 nm | between +0 | Sonication | proteins | |
| | | mV and | power: 1~50% | | |
| | | +40 mV | (for a 700 W | | |
| | | | sonicator) | | |
| | | | Sonication time: | | |
| | | | 5 s~3 mins | | |
| | | | Ionic strength of | | |
| | | | assembly | | |
| | | | solution: no | | |
| | | | requirement | | |
| | | | At least one of | | |
| | | | pre-processing | | |
| | | | methods (such | | |
| | | | as heating, | | |
| | | | sonication or | | |
| | | | quench): not | | |
| | | | required | | |
| Heart | General range: at | General | Ratio: 4.4~30 μg | siRNA, | |
| | least one dimension | range: | RNTs per | other | |
| | between 1 nm and 90 | between +0 | 0.1 nmol RNA | nucleic | |
| | nm | mV and | (Preferred ratio: | acids, | |
| | Preferred range: at | +60 mV | 6.6~20 μg RNTs | molecular | |
| | least one dimension | Preferred | per 0.1 nmol | beacons and | |
| | between 1 nm and 30 | range: | RNA) | peptides/ | |
| | nm | between +8 | Sonication | proteins | |
| | | mV and | power: | | |
| | | +40 mV | 10%~100% (for | | |
| | | | a 700 W | | |
| | | | sonicator) | | |
| | | | Sonication time: | | |
| | | | 10 s~30 mins | | |
| | | | Ionic strength of | | |
| | | | assembly | | |
| | | | solution: | | |
| | | | 0~308 mmol/L | | |
| | | | At least one of | | |
| | | | pre-processing | | |
| | | | methods (such | | |
| | | | as heating, | | |
| | | | sonication or | | |
| | | | quench): | | |
| | | | required | | |

### Diagnostic Applications

Molecular beacons or molecular beacon probes are oligonucleotide hybridization probes that report the presence of specific nucleic acids. Molecular beacons are hairpin shaped molecules with an internally quenched fluorophore whose fluorescence is restored when they bind to a target nucleic acid sequence. The use of molecular beacons is a non-radioactive method for detecting specific sequences of nucleic acids. They are useful in situations where it is either not possible or desirable to isolate the probe-target hybrids from an excess of the hybridization probes such as in the context of clinical diagnostics.

A typical molecular beacon probe is 25 nucleotides long. The middle 15 nucleotides are complementary to the target DNA or RNA and do not base pair with one another, while the five nucleotides at each terminus are complementary to each other rather than to the target DNA. A typical molecular beacon structure can be divided in 4 parts. Loop: a 18-30 base pair region of the molecular beacon that is complementary to the target sequence. Stem: the beacon stem is formed by the attachment, to both termini of the loop, of two short (5 to 7 nucleotide residues) oligonucleotides that are complementary to each other. 5′ fluorophore: located at the 5′ end of the molecular beacon, a fluorescent dye is covalently attached. 3′ quencher (non-fluorescent): the quencher dye part of the beacon is covalently attached to the 3′ end of the molecular beacon. When the beacon is in closed loop shape, the quencher resides in proximity to the fluorophore, which results in quenching the fluorescent emission of the latter.

If the nucleic acid to be detected is complementary to the strand in the loop, the event of hybridization occurs. The duplex formed between the nucleic acid and the loop is more stable than that of the stem because the former duplex involves more base pairs. This causes the separation of the stem and hence of the fluorophore and the quencher. Once the fluorophore is distanced from the quencher, illumination of the hybrid with light results in the fluorescent emission. The presence of the emission reports that the event of hybridization has occurred and hence the target nucleic acid sequence is present in the test sample. Molecular beacons are useful in SNP detection, real-time nucleic acid detection, real-time PCR quantification, allelic discrimination and identification, multiplex PCR assays, and for diagnostics. Nanopieces containing molecular beacons or other non-radioactive or radioactive detectable markers are particularly useful in diagnostic clinical assays.

### MMP

MMP13 is involved in the progression of osteoarthritis. Matrix metalloproteinase (MMP) 13 is a major enzyme that targets cartilage for degradation. Compared to other MMPs, the expression of MMP13 is relatively more restricted to connective tissue. It not only targets type II collagen in cartilage for degradation, but also degrades proteoglycan, types IV and type IX collagen, osteonectin and perlecan in cartilage. Clinical investigation revealed that patients with articular cartilage destruction have high MMP13 expression, indicating that increased MMP 13 is associated with cartilage degradation. MMP13-overexpressing transgenic mice developed a spontaneous OA-like articular cartilage destruction phenotype. The ADAMTS (a disintegrin and metalloproteinase with thrombospondin motifs) family of aggrecanases also contributes to proteoglycan/aggrecan depletion and are associated with cartilage degradation during OA. ADAMTS4 and 5 were identified as the major aggrecanases during OA development.

### ADAMTS5

ADAMTS5 is a member of the ADAMTS (a disintegrin and metalloproteinase with thrombospondin motifs) protein family and a major aggrecanase in human cartilage. Members of the family share several distinct protein modules, including a propeptide region, a metalloproteinase domain, a disintegrin-like domain, and a thrombospondin type 1 (TS) motif Individual members of this family differ in the number of C-terminal TS motifs, and some have unique C-terminal domains. The enzyme encoded by this gene contains two C-terminal TS motifs and functions as aggrecanase to cleave aggrecan, a major proteoglycan of cartilage.

ADAMTS5 plays a role in arthritis, e.g., it plays a key role in aggrecan degradation in cartilage. For example, genetically modified mice in which the catalytic domain of ADAMTS5 was deleted are resistant to cartilage destruction in an experimental model of osteoarthritis. ADAMTS5 is the major aggrecanase in mouse cartilage in a mouse model of inflammatory arthritis. ADAMTS5 is also useful as a biomarker for prediction of the response to infliximab (IFX) in patients with rheumatoid arthritis.

### Fabrication of Tissue-Targeted Nanoparticles

Examples for the preparation of nanopieces for use in individual tissues are described below.

Cartilage/chondrocytes:
1) 30 μg RNTs in 50 μL water were heated to 99° C. for 3 mins, and then mixed with 0.1 nmol MMP-13 molecular beacon. The resulting mixture was sonicated at 100% power for 10 s.
2) 4.4 μg RNTs in 1 μL water were sonicated at 50% power of a 700 W sonicator for 10 mins, and then mixed with 0.1 nmol miRNA-140. The resulting mixture was sonicated at 100% power for 30 mins.
3) 10 μg RNTs in 10 μL water were sonicated at 100% power of a 700 W sonicator for 5 mins, and then mixed with 0.1 nmol ADAMTS-5 siRNA. The resulting mixture was sonicated at 100% power for 3 mins.

Synovium:
1) 30 μg RNTs in 50 μL water were heated to 99° C. for 3 mins, and then mixed with 0.1 nmol IL-1β molecular beacon on ice. The resulting mixture was sonicated at 100% power for 60 s.
2) 4.4 μg RNTs in 1 μL saline were sonicated at 1% power of a 700 W sonicator for 10 mins, and then mixed with 0.1 nmol IL-1 receptor antagonist protein. The resulting mixture was sonicated at 1% power for 10 s.
3) 10 μg RNTs in 10 μL water were sonicated at 100% power of a 700 W sonicator for 5 mins, and then mixed with 0.1 nmol TNF-α siRNA. The resulting mixture was sonicated at 100% power for 60 s.

Neurons:
1) 15 μg RNTs in 50 μL water at 1% power of a 700 W sonicator for 30 mins, and then mixed with 0.1 nmol VEGF molecular beacon. The resulting mixture was sonicated at 100% power for 60 s.
2) 0.1 μg RNTs in 1 μL saline were heated to 99° C. for 3 mins, and then mixed with 0.1 nmol IL-1 receptor siRNA on ice. The resulting mixture was sonicated at 100% power for 30 mins.
3) 10 μg RNTs were sonicated in 10 μL water at 100% power of a 700 W sonicator for 5 mins, and then mixed with 0.1 nmol MMP-1 siRNA. The resulting mixture was sonicated at 100% power for 3 mins.

Brain/BBB:
1) 20 μg RNTs in 50 μL water were heated to 99° C. for 3 mins, and then mixed with 0.1 nmol MMP-9 molecular beacon on ice. The resulting mixture was sonicated at 100% power for 60 s.
2) 1 μg RNTs in 1 μL saline were sonicated at 10% power of a 700 W sonicator for 10 mins, and then mixed with 0.1 nmol VEGF mRNA. The resulting mixture was sonicated at 10% power for 10 s.
3) 10 μg RNTs in 10 μL water were sonicated at 100% power of a 700 W sonicator for 5 mins, and then mixed with 0.1 nmol TNF-α siRNA. The resulting mixture was sonicated at 100% power for 60 s.

Ocular tissue:
1) 30 μg RNTs in 50 μL water were heated to 99° C. for 3 mins, and then mixed with 0.1 nmol VEGF molecular beacon on ice. The resulting mixture was sonicated at 100% power for 60 s.
2) 4.4 μg RNTs in 1 μL saline were sonicated at 1% power of a 700 W sonicator for 10 mins, and then mixed with 0.1 nmol VEGF antagonist protein. The resulting mixture was sonicated at 1% power for 10 s.
3) 10 μg RNTs in 10 μL water were sonicated at 100% power of a 700 W sonicator for 5 mins, and then mixed with 0.1 nmol VEGF siRNA. The resulting mixture was sonicated at 100% power for 60 s.

Derm tissue/skin:
1) 30 μg RNTs in 50 μL water were heated to 99° C. for 3 mins, and then mixed with 0.1 nmol IL-1β molecular beacon on ice. The resulting mixture was sonicated at 100% power for 60 s.
2) 4.4 μg RNTs in 1 μL saline were sonicated at 1% power of a 700 W sonicator for 10 mins, and then mixed with 0.1 nmol IL-6 siRNA. The resulting mixture was sonicated at 1% power for 10 s.
3) 10 μg RNTs in 10 μL water were sonicated at 100% power of a 700 W sonicator for 5 mins, and then mixed with 0.1 nmol IL-8 siRNA. The resulting mixture was sonicated at 100% power for 60 s.

Tumor:
1) 30 μg RNTs in 50 μL water at 1% power of a 700 W sonicator for 30 mins, and then mixed with 0.1 nmol VEGF molecular beacon. The resulting mixture was sonicated at 100% power for 60 s.
2) 0.1 μg RNTs in 1 μL saline were heated to 99° C. for 3 mins, and then mixed with 0.1 nmol TNF-α siRNA on ice. The resulting mixture was sonicated at 100% power for 30 mins.
3) 10 μg RNTs were sonicated in 10 μL water at 100% power of a 700 W sonicator for 5 mins, and then mixed with 0.1 nmol MMP-1 siRNA. The resulting mixture was sonicated at 100% power for 3 mins.

Kidney:
1) 30 μg RNTs in 50 μL water were heated to 99° C. for 3 mins, and then mixed with 0.1 nmol IL-12 molecular beacon on ice. The resulting mixture was sonicated at 100% power for 60 s.
2) 4.4 μg RNTs in 1 μL saline were sonicated at 5% power of a 700 W sonicator for 10 mins, and then mixed with 0.1 nmol IL-1 receptor associated protein siRNA. The resulting mixture was sonicated at 1% power for 10 s.
3) 10 μg RNTs in 10 μL water were sonicated at 100% power of a 700 W sonicator for 5 mins, and then mixed with 0.1 nmol IL-8 siRNA. The resulting mixture was sonicated at 100% power for 60 s.

Mucous membrane:
1) 30 μg RNTs in 50 μL water were heated to 99° C. for 3 mins, and then mixed with 0.1 nmol MMP-13 molecular beacon on ice. The resulting mixture was sonicated at 100% power for 60 s.
2) 4.4 μg RNTs in 1 μL saline were sonicated at 1% power of a 700 W sonicator for 10 mins, and then mixed with 0.1 nmol MMP-9 siRNA. The resulting mixture was sonicated at 1% power for 10 s.
3) 10 μg RNTs in 10 μL water were sonicated at 100% power of a 700 W sonicator for 5 mins, and then mixed with 0.1 nmol MMP-1 siRNA. The resulting mixture was sonicated at 100% power for 60 s.

Lung:
1) 30 μg RNTs in 50 μL water were heated to 99° C. for 3 mins, and then mixed with 0.1 nmol TNF-α molecular beacon on ice. The resulting mixture was sonicated at 50% power for 60 s.
2) 4.4 μg RNTs in 1 μL saline were sonicated at 1% power of a 700 W sonicator for 3 mins, and then mixed with 0.1 nmol MMP-9 siRNA. The resulting mixture was sonicated at 1% power for 5 s.
3) 10 μg RNTs in 10 μL water were sonicated at 50% power of a 700 W sonicator for 1 mins, and then mixed with 0.1 nmol MMP-1 siRNA. The resulting mixture was sonicated at 100% power for 60 s.

Heart:
1) 30 μg RNTs in 50 μL water were heated to 99° C. for 3 mins, and then mixed with 0.1 nmol VEGF molecular beacon. The resulting mixture was sonicated at 100% power for 10 s.
2) 4.4 μg RNTs in 1 μL water were sonicated at 50% power of a 700 W sonicator for 10 mins, and then mixed with 0.1 nmol miRNA-365. The resulting mixture was sonicated at 100% power for 30 mins.
3) 10 μg RNTs in 10 μL water were sonicated at 100% power of a 700 W sonicator for 5 mins, and then mixed with 0.1 nmol IL-1α siRNA. The resulting mixture was sonicated at 100% power for 3 mins.

Coating of Nanopieces, which is another important factor for tissue delivery can also be used to improve the tissue delivery. For example polyethylene glycol (PEG) and dextran are coatings often used.

The invention further provides methods for making composites of rosette nanotubes or components or rosette nanotubes or rosette Nanopieces and therapeutic or diagnostic agents including those known in the art. For example, agents include nucleic acids (DNA or RNA), wherein the RNA can be small RNA such as siRNA and miRNA. In particular, disclosed herein are novel siRNA transport complexes, comprising an unexpectedly advantageous transport vehicle. Methods of the present invention include contacting a transfection complex described herein with one or more cells, where the transfection complex includes a rosette nanotube and one or more nucleic acids such as DNA and RNA, for example siRNA. The rosette nanotube is a carrier that is formed from self-assembled modules as described below and those modules recognized in the art.

### Compounds/Modules for Self-Assembly

Modules according to the present disclosure include compounds of Formula I below:

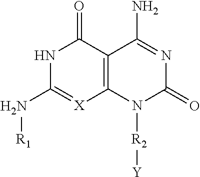

Wherein X is CH or nitrogen, preferably nitrogen; R₂ is hydrogen or a linker group for example (CH₂)ₙ or other linker groups described herein, preferably (CH₂)ₙ; n is an integer of, 1, 2, 3, or 4, n=2 is preferred; Y is absent when R₂ is hydrogen or is an amino acid or polypeptide having an amino group covalently bound to an α-carbon of the amino acid and the amino group is covalently bound to the linker group R₂, Y is preferred to be lysine arginine, and histidine; and R₁ is hydrogen or an aliphatic moiety, such as alkyl, straight or branched chain, saturated or unsaturated; and salts thereof. Preferably R₁ is C₁ to C₁₀ alkyl, C₁ to C₅ alkyl, C₁ to C₃ alkyl, or methyl. Compounds within the scope of the invention include those where the Y group can be connected to the linker group either by the amino group or the carboxyl group of the amino acid or polypeptide. An exemplary linker group is shown in the formula below.

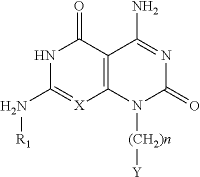

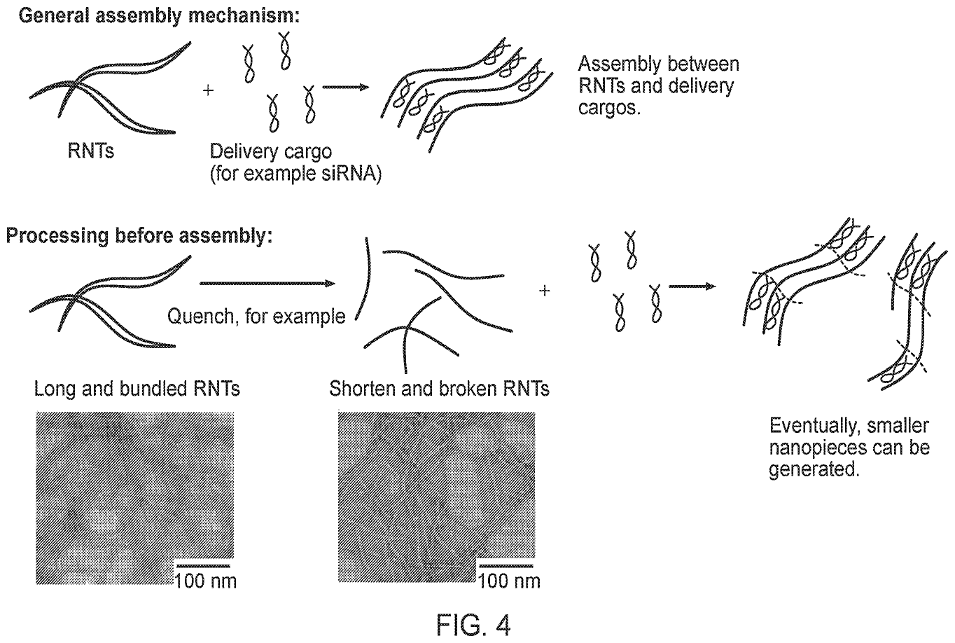

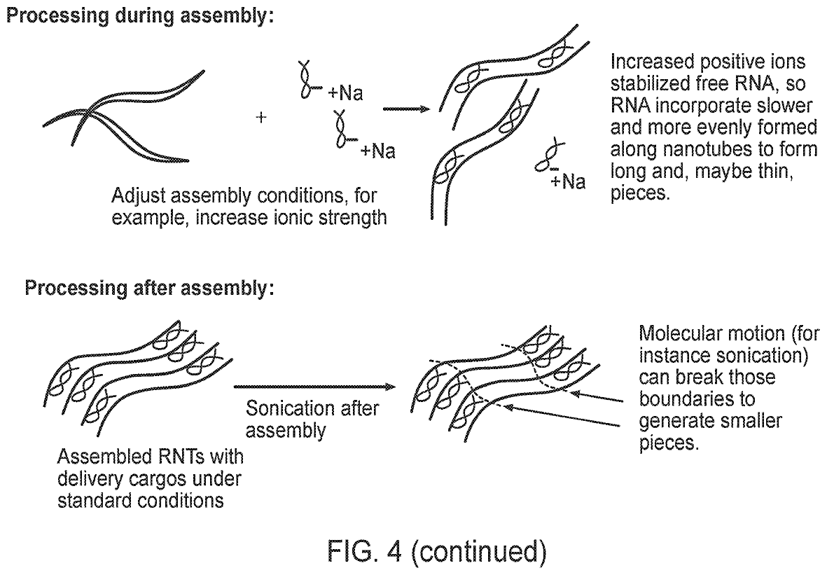

An exemplary module within the scope of Formula I is shown in FIG. 4 along with a schematic representation of a nanotube and an image of nanotubes formed from the exemplary module.

Alternative linker groups R₂ can join the Y group to the carbon of the (CH₂)ₙ group or the N atom either by the amino group or the carboxyl group of the amino acid or polypeptide.

Alternative R₂ groups within the scope of the present disclosure are selected from a group comprising:

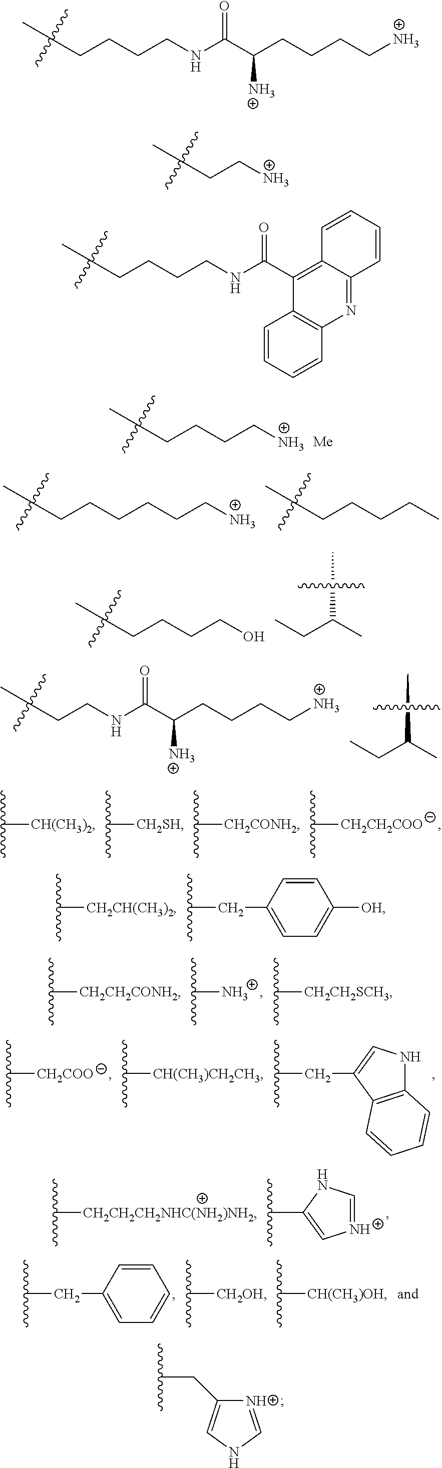

wherein Y is absent.

Compounds of Formula I can be prepared by the methods described in U.S. Pat. No. 6,696,565 hereby incorporated by reference herein in its entirety alone or combined with methods known to those of skill in the art. Rosette nanotubes are made by assembly of compounds of Formula (I).

Exemplary compounds of Formula I are shown below:

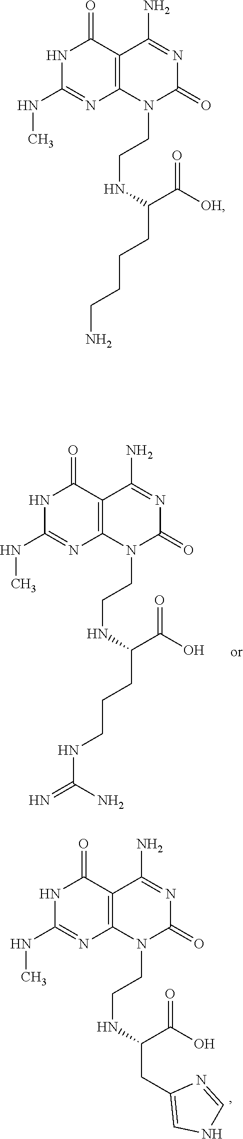

and a salt thereof. In embodiments, the compounds of Formula I are present in zwitterion form. The compounds are shown as below.

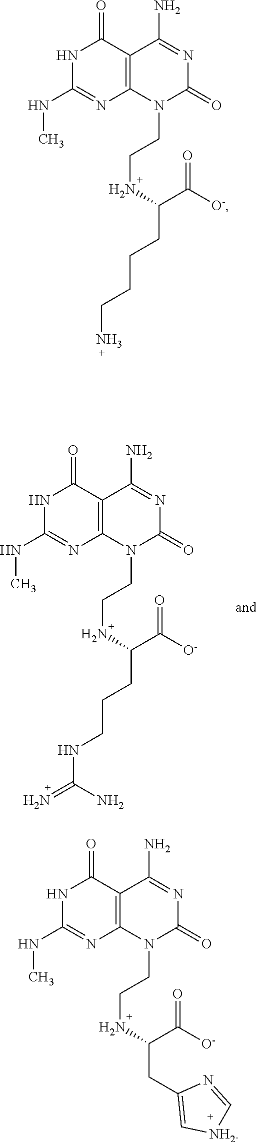

The salt forms thereof may be presented as follows:

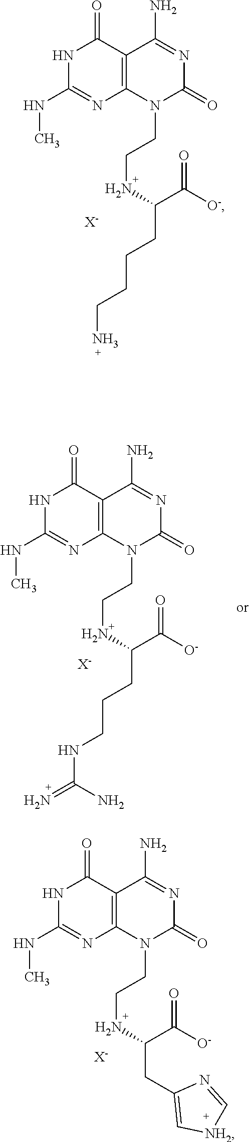

wherein X⁻ can be anion such as Cl⁻, or other anionic organic acids.

Modules according to the present disclosure also include compounds of Formula II below:

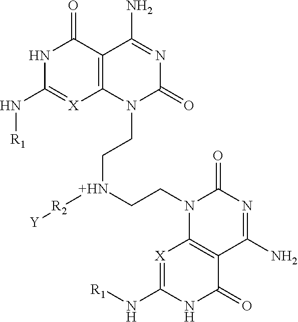

Wherein X is CH or nitrogen preferably nitrogen; R₂ is hydrogen or a linker group for example (CH₂)ₙ, preferably (CH₂)ₙ; where n is an integer of, 1, 2, 3, or 4 or (CH₂)₃C₀ or other linker groups described herein, n=2 is preferred; Y is absent when R₂ is hydrogen or is an amino acid or polypeptide having an amino group covalently bound to an α-carbon of the amino acid and the amino group is covalently bound to the linker group R₂, Y is preferred to be lysine arginine, and histidine; and R₁ is hydrogen or an aliphatic moiety, such alkyl, straight or branched chain, saturated or unsaturated; and salts thereof. Preferably R₁ is C₁ to C₁₀ alkyl, C₁ to C₅ alkyl, C₁ to C₃ alkyl, or methyl. An exemplary linker group is shown in the formula below.

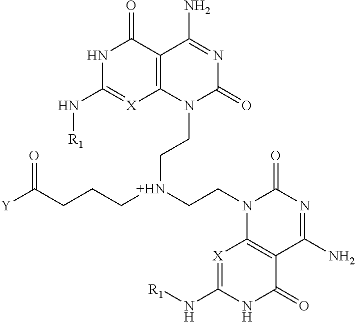

Compounds within the scope of the present disclosure include those where the Y group can be connected to the linker group either by the amino group or the carboxyl group of the amino acid or polypeptide. Alternative R₂ groups within the scope of the present disclosure are selected from a group comprising:

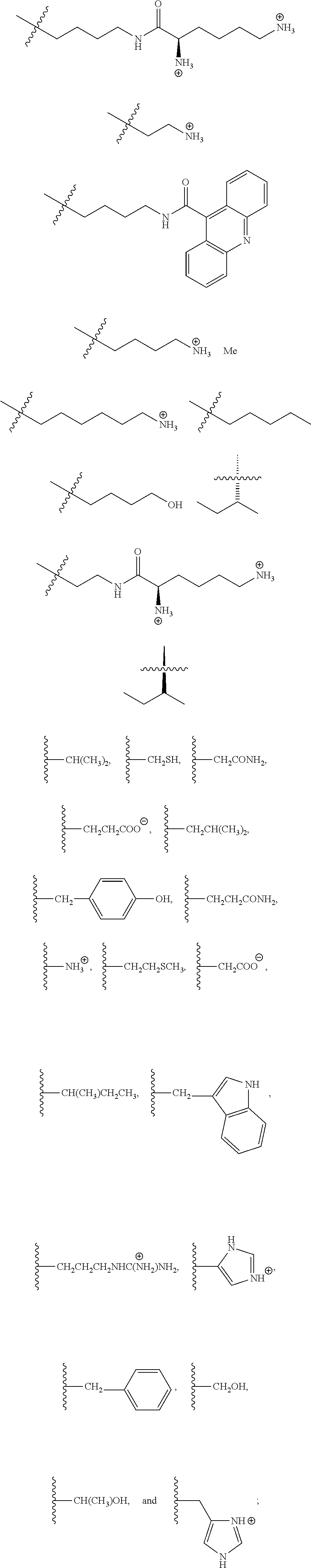

wherein Y is absent. TBL structures are made by the assembly of compounds of Formula (II).

Exemplary compounds of Formula II are shown below:

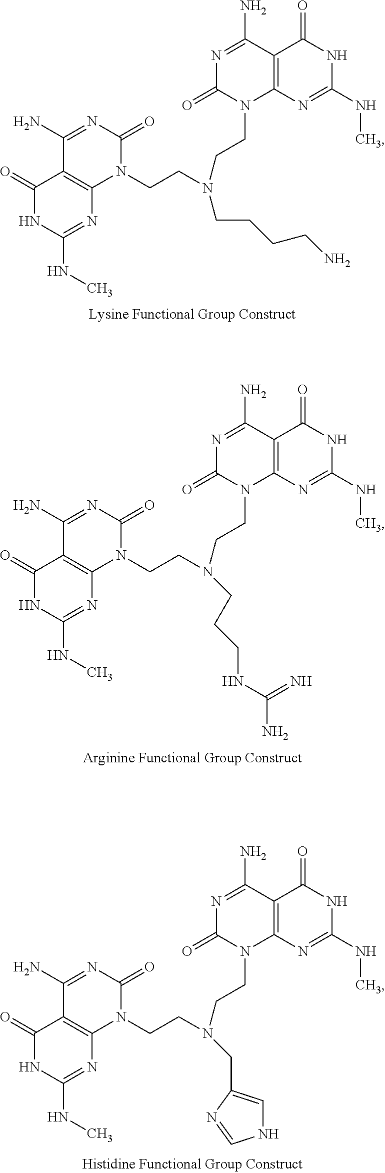 [SMILES: CNc1nc2c(c(N)nc(=O)n2CCN(CCCCN)CCn2c(=O)nc(N)c3c(=O)[nH]c(NC)nc32)c(=O)[nH]1 ; CNc1nc2c(c(N)nc(=O)n2CCN(CCCNC(=N)N)CCn2c(=O)nc(N)c3c(=O)[nH]c(NC)nc32)c(=O)[nH]1 ; CNc1nc2c(c(N)nc(=O)n2CCN(CCn2c(=O)nc(N)c3c(=O)[nH]c(NC)nc32)Cc2c[nH]cn2)c(=O)[nH]1]

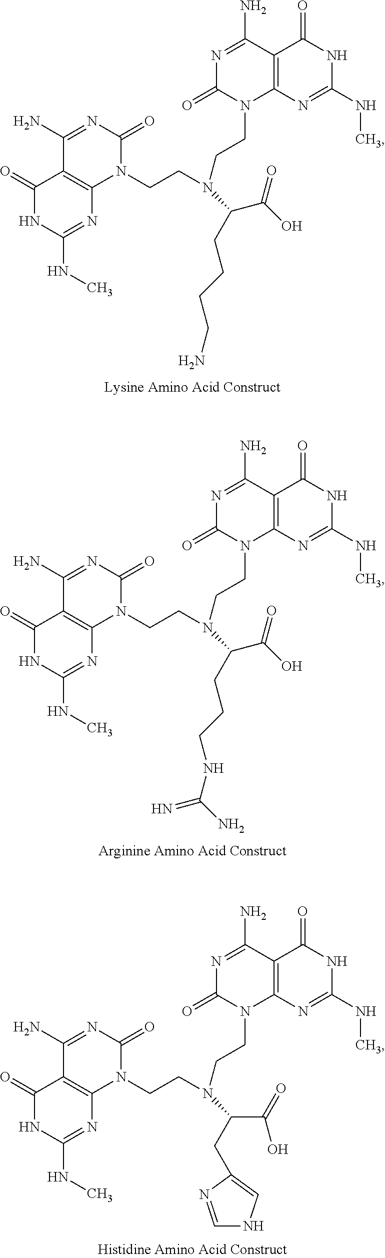 [SMILES: CNc1nc2c(c(N)nc(=O)n2CCN(CCn2c(=O)nc(N)c3c(=O)[nH]c(NC)nc32)[C@@H](CCCCN)C(=O)O)c(=O)[nH]1 ; CNc1nc2c(c(N)nc(=O)n2CCN(CCn2c(=O)nc(N)c3c(=O)[nH]c(NC)nc32)[C@@H](CCCNC(=N)N)C(=O)O)c(=O)[nH]1 ; CNc1nc2c(c(N)nc(=O)n2CCN(CCn2c(=O)nc(N)c3c(=O)[nH]c(NC)nc32)[C@@H](Cc2c[nH]cn2)C(=O)O)c(=O)[nH]1]

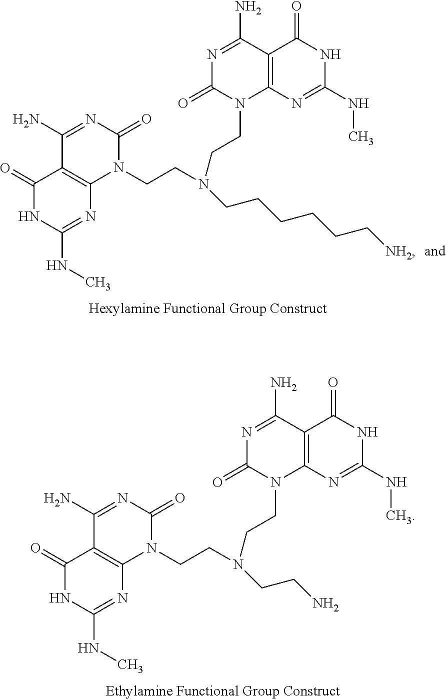 [SMILES: CNc1nc2c(c(N)nc(=O)n2CCN(CCCCCCN)CCn2c(=O)nc(N)c3c(=O)[nH]c(NC)nc32)c(=O)[nH]1 ; CNc1nc2c(c(N)nc(=O)n2CCN(CCN)CCn2c(=O)nc(N)c3c(=O)[nH]c(NC)nc32)c(=O)[nH]1]

In some embodiments, compounds of formula II comprise amino acid functional group constructs. These compounds contain functional groups present in natural occurring amino acid side chains or may contain the entire amino acid side chain. For example, the lysine functional group construct contains the entire amino acid side chain functionality (—CH₂CH₂CH₂CH₂NH₃⁺), whereas the histidine functional group construct only contains the heteroaryl imidazole group present in histidine.

In some embodiments, compounds of formula II comprise amino acid analogs. These compounds contain the entire the amino acid or may contain modified and/or unnatural amino acids. For example, the lysine amino acid analog contains the entire amino acid functionality of lysine, whereas the histidine amino acid analog contains a modified histidine amino acid.

In some embodiments the compound of formula II is the Lysine Functional Group Construct:

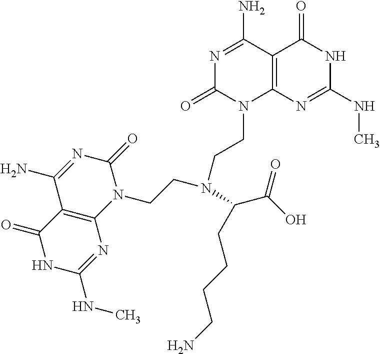 [SMILES: CNc1nc2c(c(N)nc(=O)n2CCN(CCn2c(=O)nc(N)c3c(=O)[nH]c(NC)nc32)[C@@H](CCCCN)C(=O)O)c(=O)[nH]1]

According to certain aspects of the present disclosure, the structure of Formula II is referred to as a twin base with a linker (TBL) or twin base linkers insofar as two similar double ring structures are present as shown in Formula II and are linked to an amino acid or polypeptide. However, it is to be understood that the two double ring structures need not be identical insofar as they may have different X and R₁ groups.

Embodiments of the present disclosure involve making composites of rosette nanotubes or components or rosette nanotubes or rosette Nanopieces and therapeutic or diagnostic agents including those known in the art and including nucleic acids, such as DNA or RNA. RNA can be small RNA including siRNA and miRNA. In particular, disclosed herein are novel siRNA transport complexes, comprising an unexpectedly advantageous transport vehicle. Methods of the present invention include contacting a transfection complex described herein with one or more cells, where the transfection complex includes a rosette nanotube and one or more nucleic acids such as DNA and RNA, for example siRNA. The rosette nanotube is a carrier that is formed from self-assembled modules as described below and those modules are recognized in the art.

TBL or twin base linkers comprise structures shown in Formula II and are linked to an amino acid, amino acid side chain structure, or polypeptide; compounds of Formula I may also be linked to an amino acid, amino acid side chain structure, or polypeptide. However, it is to be understood that the two double ring structures need not be identical insofar as they may have different X, Y, and R₁ groups.

Amino acids can be divided into amino acid containing hydrophilic side chains, hydrophobic side chains, and electrically charged side chains. See chart below, wherein the side chains are shaded:

According to aspects of the present disclosure, modules (compounds) according to Formula I and Formula II self-assemble into substructures also called supermacrocycles which themselves will self-assemble into nanometer scale architectures or structures such as discrete nanotubular assemblies in water or aqueous solutions. Supermacrocycles are defined herein as being a number of organic molecules covalently or noncovalently bound together so as to form a ring structure. For example, compounds of Formula I will self-assemble into a 6-mer ring structure, sometimes referred to as a rosette. The process of forming nanotubes with the modules of the present disclosure is hierarchical. In particular, the modules of the present invention first self-assemble into supermacrocycles, and then the supermacrocycles self-assembly into nanotubes. Such self-assembly is described in U.S. Pat. No. 6,696,565. For the compounds of Formula II referred to as twin base linkers, the compounds will also assemble into a 6-mer ring structure. However, a single supermacrocycle formed will include two base layers owing to the presence of the two bases in each of the compound of Formula II.

Examples of modules of the present disclosure comprise the compounds of Formula I and Formula II and may include low molecular weight synthetic DNA base analogues referred to by the nomenclature C\hat{ }G (Fenniri et al, J. Am. Chem. Soc. 2001, 123, 3854-3855) and A\hat{ }T. The C\hat{ }G moiety, referred to as a single CG motif, possesses the Watson-Crick donor-donor-acceptor of guanine and the acceptor-acceptor-donor of cytosine and undergoes a self-assembly process, fueled by an array of hydrogen bonds, to produce a six-membered supermacrocycle or rosette. Stacking of these rosettes produced a nanotube of very high aspect ratio. Compounds within the scope of the present invention include a twin G\hat{ }C motif denoted as (C\hat{ }G)₂. Like the single C\hat{ }G motif, the twin C\hat{ }G motif (C\hat{ }G)₂ also possesses the Watson-Crick donor-donor-acceptor of guanine and the acceptor-acceptor-donor of cytosine and undergoes a self-assembly process, fueled by an array of hydrogen bonds, to produce a six-membered supermacrocycle or ring structure (rosette) of twin configuration. Stacking of these twin rosettes produces a nanotube of very high aspect ratio and higher stability. Analogously, The A\hat{ }T moiety, referred to as a single AT motif, also possesses the Watson-Crick donor-donor-acceptor of adenine and the acceptor-acceptor-donor of thymine and undergoes a self-assembly process as well, fueled by an array of hydrogen bonds, to produce a six-membered supermacrocycle or rosette. Stacking of these rosettes produces a nanotube of very high aspect ratio. Compounds within the scope of the present invention include a twin A\hat{ }T motif denoted as (A\hat{ }T)₂. Like the single A\hat{ }T motif, the twin A\hat{ }T motif (A\hat{ }T)₂ also possesses the Watson-Crick donor-donor-acceptor of adenine and the acceptor-acceptor-donor of thymine and undergoes a self-assembly process, fueled by an array of hydrogen bonds, to produce a six-membered supermacrocycle or ring structure (rosette) of twin configuration. Stacking of these twin rosettes also produces a nanotube of very high aspect ratio and higher stability.

It should be understood that the above described Formula I and/or Formula II demonstrate that electrostatic, stacking and hydrophobic interactions can be effectively orchestrated by hydrogen bonds to direct the hierarchical assembly and organization of helical nanotubular architectures in an aqueous milieu. Helical nanotubular architectures within the scope of the present invention include those formed entirely from compounds of Formula I. Helical nanotubular architectures within the scope of the present invention include those formed entirely from compounds of Formula II. Further, helical nanotubular architectures within the scope of the present invention include those formed from one or more of the compounds of Formula I and one or more of the compounds of Formula II. For example, a supermacrocycle ring substructure having particular amino acid or polypeptide side chains formed from the compounds of Formula I can be stacked with a supermacrocycle ring substructure having particular amino acid or polypeptide side chains formed from compounds of Formula II. The rosette substructures formed from the compounds of Formula I and Formula II can be stacked in any desired sequence to form nanotubular structures of the present invention. Utilizing this aspect of the present invention, a wide variety of structurally different modules (e. g, compounds) can be synthesized and self-assembled into supermacrocycles and then nanotubular structures according to methods of the present invention.

Another aspect of the invention is the conversion of nanotubes to nanorods by altering pH, temperature, and usage of physical methods (e.g., sonication, heating and blending) to prepare different sizes of Nanopieces.

Before assembly with delivery cargo, length of nanotubes (based on either Formula I or II) range in size from 1 nm to 999 micron, e.g., 10 nm to 999 nm. Outer width of nanotubes range in size from 0.5 nm to 100 nm, e.g., 1 nm to 10 nm. Inner diameter of nanotubes range in size from 1 angstrom to 10 nm, e.g., 0.5 nm to 5 nm.

After assembly with delivery cargo, length of Nanopieces (based on either Formula I or II) range in size from 1 nm to 999 micron, e.g., 10 nm to 999 nm. Width of Nanopieces range in size from 1 nm to 999 nm, e.g., 10 nm to 100 nm.

Another aspect of the invention is the packaging of drug molecules, e.g., therapeutics and diagnostics, with nanotubes to alter their surface charge and more importantly process these nanotubes into Nanopieces of the right shape and size to penetrate tissue matrix. Therefore, it is not the drug molecules that are released from nanotubes that diffuse into tissue, it is the Nanopieces themselves that penetrate the tissue. Control of the surface charge of the Nanopieces is done via the ratio of delivery cargo and nanotubes and/or nanorods. A further aspect of the invention is the use of coatings for the Nanopieces for tissue delivery. For example, polyethylene glycol and/or dextran are coatings that when used can improve tissue delivery.

A further aspect of the invention is the delivery of cargo into cells. These drug molecules can be nucleic acid, peptides, proteins, aromatic small molecules or negatively charged small molecules.

In some embodiments, the prepared module of the invention has an overall yield of no less than 60%, e.g., no less than 70%, no less than 80%, or no less than 90%.

In some embodiments, the module of the method of the invention contains more than 80% of compound of Formula I or II. In some embodiments, the product of the method of the invention contains more than 85%, 90%, 92%, 95%, 97%, 98%, 98.5%, or 99% of compound of Formula I and/or II. For example, the product is free of undesired byproduct or starting material.

In some embodiments, the nanotube of the invention has an overall yield of no less than 60%, e.g., no less than 70%, no less than 80%, or no less than 90%.

In some embodiments, the nanotube of the method of the invention contains more than 80% of compound of Formula I or II. In some embodiments, the product of the method of the invention contains more than 85%, 90%, 92%, 95%, 97%, 98%, 98.5%, or 99% of compound of Formula I and/or II. For example, the product is free of undesired byproduct or starting material.

In some embodiments, the Nanopieces of the invention has an overall yield of no less than 60%, e.g., no less than 70%, no less than 80%, or no less than 90%.

In some embodiments, the Nanopieces of the method of the invention contains more than 80% of compound of Formula I or II. In some embodiments, the product of the method of the invention contains more than 85%, 90%, 92%, 95%, 97%, 98%, 98.5%, or 99% of compound of Formula I and/or II. For example, the product is free of undesired byproduct or starting material.

According to certain preferred aspects of the present invention, a nanotube is prepared from single base ring structures and twin base ring structures in any desired order. The nanotube can have one or more single base ring structures and one or more twin base ring structures. Likewise, a nanotube within the scope of the present invention can include a plurality of single base ring structures formed from compounds of Formula I and a plurality of twin base ring structures formed from compounds of Formula II stacked together, e.g. one next to the other via hydrogen bonding, to form the nanotube.

### Nanotube-Agent Complexes

According to certain aspects, nucleic acids or polypeptides includes small RNA being a duplex of between about 10 to about 30 nucleic acids, between about 15 to about 25 nucleic acids and between about 20 to about 23 nucleic acids, and any values and ranges in between whether overlapping or not. The small RNA can be formed by one or more oligonucleotides. Small RNA includes RNA commonly referred to as interference RNA, dsRNA, ssRNA, saRNA, siRNA or miRNA or their derivatives, analogs, mimics and inhibitors. According to certain aspects, siRNA is involved in the RNA interference (RNAi) pathway, where it interferes with the expression of a specific gene. In addition to their role in the RNAi pathway, siRNAs also act in the RNAi-related pathways. siRNA within the scope of the present disclosure includes double stranded RNA of about 21 nucleotides with a 2 nucleotide 3′ overhang on either end of the siRNA. Each siRNA strand has a 5′ phosphate group and a 3′ hydroxyl (—OH) group. The structure is the result of processing by dicer, an enzyme that converts either long dsRNAs or small hairpin RNAs into siRNAs. Particular exemplary sequences of siRNA are readily available to those of skill in the art through published literature and siRNA is commercially available from, for example, Qiagen. It is to be understood that the present disclosure is not to be limited to any particular siRNA sequence, but rather the present disclosure broadly describes the incorporation of siRNA into or with rosette nanotubes. One of skill in the art will readily recognize that all siRNA sequences, given the similar structure and function of covalently connected nucleotides, can be incorporated into or complexed with rosette nanotubes using the methods described herein and that an exhaustive listing of publicly known siRNA sequences need not be provided herein.

According to additional aspects, DNA includes any DNA desired to be expressed by a cell. DNA includes genes having known functions and expressing known proteins. Likewise, DNA suitable for transfecting a cell will be apparent to those of skill in the art of transfection and gene expression.

### Manufacture and Use of Transfection Complexes

The present disclosure is directed to methods of forming a transfection complex, for example, by mixing one or more nucleic acids with fully formed rosette nanotubes or modules that self-assemble into rosette nanotubes, such as the compounds of Formula I or Formula II. According to one aspect, fully formed rosette nanotubes in the form of a powder is dissolved in water and heated to boiling. The solution is then cooled to room temperature. One or more nucleic acids in the form of a solution is then added to the solution of nanotubes at a suitable temperature and for a suitable period of time until a complex of the nanotube and one or more nucleic acids forms. Suitable ratios of the nucleic acid to nanotube include about 0.01:1 (wt/wt) to about 1:0.1 (wt/wt).

The invention is further directed to transfection complexes, which include small RNA, such as siRNA and a rosette nanotube. Transfection complexes in accordance with the present invention may include any of the rosette nanotubes of the present invention in combination with small RNA known to those of skill in the art.

According to certain aspects, cells within the scope of the present invention that can be transfected include osteoblasts, fibroblasts, stem cells, neuronal cells, connective tissue cells, keratinocytes, cardiac myocytes, chondrocytes, proteoglycans, synoviocytes, adipose, phagocytic, blood monocytes, mesenchymal stem cells, neural stem cells, islet cells, hepatocytes, smooth muscle cells, urothelial cells, neurons, Schwann cells, microgial cells, cancerous and non-cancerous cells, epithelial cells, endothelial cells, myofibroblasts, osteoclasts, macrophages, leukocytes, osteocytes, astrocytes etc. and the like. Additional cells include bacterial cells such as Staphylococcus aureus, Staphylococcus epidermis, Pseudomonas aeruginosa, MRSA, E. coli, candida (yeast), Candida albacans, Streptococcus pneumoniae, Neisseria meningitides, Haemophilus influenzae, Streptococcus agalactiae, Listeria monocytogenes, Mycoplasma pneumoniae, Chlamydia pneumoniae, Legionella pneumophila, Mycobacterium, tuberculosis, Streptococcus pyogenes, Chlamydia trachomatis, Neisseria gonorrhoeae, Treponema pallidum, Ureaplasma urealyticum, Haemophilus ducreyi, Helicobacter pylori, Campylobacter jejuni, Salmonella, Shigella, Clostridium, Enterobacteriaceae, Staphylococcus saprophyticus and the like. The above list is intended to be exemplary and not exhaustive. One of skill in the art will readily be able to identify additional cells within the scope of the present disclosure, which is directed to toward cells present in joints, tissue and/or organs.

In general, a cell to be transfected includes, but is not limited to, any animal, plant or bacterial cell that is susceptible to intracellular delivery of DNA or RNA such as siRNA using the transfection complex of the present invention either in vitro or in vivo. For example, cells from different species such as human, mouse, rat, pig, chicken, etc. may be used according to the present disclosure. Likewise, cells from different tissues or organs, such as cartilage (e.g, ear, nose, rib cage, bronchial tube, intervertebral disc, hyaline, fibrous, elastic), connective tissue (e.g. loose, dense, adipose, fibrous, elastic, lymphoid), conjunctive tissue, fibers (e.g., collagenous, elastic, reticular), synovium, neuronal tissue, muscle tissue, ligament, tendon, busae, fibroblast, beast cells, macrophages from the immune system, and astrocytes from the neuronal system may be used. Likewise, primary cells obtained directly from animals, plants or bacteria may be used and cell lines, such as commercially available immortalized cell, may be used. Likewise, normal cells may be used and diseased cells may be used, such as cancer cells. For example, suitable cellular targets include, without limitation, epithelial cells, endothelial cells, keratinocytes, fibroblasts, muscle cells, hepatocytes, blood cells such as T lymphocytes, B lymphocytes, monocytes, macrophages, neutrophils, eosinophils, megakaryocytes, granulocytes, various stem or progenitor cells, in particular hematopoietic stem or progenitor cells, e.g., as obtained from bone marrow, umbilical cord blood, peripheral blood, fetal liver, and the like. In certain aspects, the cell is selected from the group consisting of synoviocytes, fibroblasts, monocytes, chondrocytes, collagen, endothelial cells, connective tissue cells, neuronal cells, muscle cells, hematopoietic stem cells and tumor cells.

According to certain embodiments, the cells include one or more cells selected from the group consisting of transformed, recombinant, malignant, and primary cell lines. It is believed that the rosette nanotubes of the present invention will be effective as carriers of DNA or RNA such as siRNA in most, if not all cell types and cell lines. Since complexes of the rosette nanotubes and nucleic acids are composed of covalently bound base pairs, one of skill would expect that such complexes will be universally recognized by all cell types for transfecting purposes.

Methods of transfecting cells in accordance with the present invention may also include forming the transfection complex by combining in aqueous media the modules of the rosette nanotube and one or more DNA sequences and/or one or more RNA sequences. The complex is allowed to form. Cells are then contacted with the complex. According to one aspect, one of skill in the art will recognize from the benefit of the present disclosure that doses, concentrations, ratios and conditions of RNT/nucleic acids incorporation can be within ranges. For example, between about 1 μL to about 100 μL, for example 10 μL, of 1 mg/mL RNTs can be mixed with about 1 μL to about 100 μL, for example 20 μL, of 5 μM nucleic acids, such as siRNA, miRNA, nucleic acid probes or other nucleic acids, at a temperature of between about 0° C. to about 37° C. for between about 0.5 hours to about 48 hours and added into 1 mL cell culture medium for transfection. For example, the combination of RNT and nucleic acids can be maintained at 4° C. for 24 hours or can be maintained at room temperature for two hours. Mixing can be accomplished by simple mixing, mixing while heating to about 60° C. to about 100° C., sonication or other methods known to those of skill in the art. If heated, the combination may then be subjected to a temperature of between about 0° C. to about 37° C. for between about 0.5 hours to about 48 hours to result in formation or assembly of the nanotube/nucleic acid complex. For example, nanotubes can be modified to modulate the surface charge of the nanotubes comprising one or more DNA sequence and/or one or more RNA sequences by varying the RNT/nucleic acid ratio. A skilled person in the arts would recognize that cartilage, for example, is a negatively charged tissue matrix and nanotube carrying an overall positive charge would increase the residence time of such Nanopieces in cartilage tissue.

### Method of Treatment

The present invention also provides methods of treating tissue, organ and/or joint disease comprising using the complexes or compositions of the present invention. In particular, methods are provided for treating a patient having a tissue, organ or joint disease, by administering to the patient a therapeutically effective amount of a complex or composition of the present invention. For in vivo therapies based on local injection (e.g., intra-articularly, intratumoral, and intramuscularly) the RNT/small RNA complex is advantageously water soluble and so may be administered as an aqueous injection.

According to aspects of the present disclosure, composites of rosette nanotubes and small RNA can be combined with a pharmaceutically acceptable agent and administered as a delivery composition to an individual for therapeutic purposes.

In accordance with certain examples, complexes of the present invention can be incorporated into pharmaceutical compositions suitable for administration. Such compositions typically comprise the complexes disclosed here and a pharmaceutically acceptable carrier. As used herein the term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

### Therapeutic Applications

Also encompassed are methods for treating a patient having a tissue, organ and/or joint disease, by administering to the patient cells that have been transfected by the methods disclosed herein. An aspect of an ex vivo delivery method of the present invention may include for example, (i) removing a cell from a subject; (ii) introducing siRNA into a cell by contacting the cell with a delivery composition (transfection complex or composition comprising such a transfection complex) comprising siRNA and a rosette nanotube; and (iii) reintroducing the cell into the subject. In addition, nanotubes having nucleic acids complexed therewith as described herein may be delivered in vivo to an individual in need of treatment where the nanotubes having nucleic acids complexed therewith enter cells within the individual and the nucleic acids regulate cellular expression of proteins. For example the nucleic acids may silence genes in a therapeutic manner to the extent that a protein is not expressed resulting in treatment or the nucleic acids may be expressed by the cell to produce proteins in a therapeutic manner resulting in treatment.

Examples of joint diseases (e.g. synovial, fibrous, cartilagenoius) potentially treatable with the complex, compositions, and methods include, but are not limited to the following: autoimmune, degenerative, inflammatory, infectious, cancerous, viral, fungal, injured or trauma derived. These joint diseases may be the primary disease or may be caused by an existing disease and/or illness. Examples include polymyalgia rheumatica, rheumatoid arthritis, multiple sclerosis, Charcot's Joint, osteoarthritis, juvenile onset of rheumatoid arthritis (JRA), system lupus erythematosus (SLE), psoriatic arthritis, inflammatory bowel disease (IBS) S) arthritis, Whipple's disease, intestinal lipodystrupjy, ankylosing spondylitis (AS), reactive arthritis, Still's disease, avascular necrosis, bursitis, fibromyalgia, gout, hemochromatosis, hypothyroidism, lupus, Lyme disease, Fifths disease, osteomalacia, osteomyelitis, Paget's disease of bone, pseudogout, rickets, septic arthritis, tendinitis, diabetes, Ehlers-Danlos syndrome, costochondritis, Perthes' disease, Marfan syndrome, rheumatic fever, tubercular arthritis, pigmented villonodular synovitis, scleroderma, polymyositis, erythema nodosum, neuropathic arthropathy, sickle-cell disease, acromegaly, amyloidosis, acute crystal synovitis, pyogenic bacterial infection, scurvy, hemophilia, achondroplasia, herniation, diffuse iodophatic skeletal hyperostosis (DISH), ganglion, lumbar spinal stenosis, sacrolilac joint pain, SAPHO syndrome, polycythemia, Raynaud's phenomenon, hydroxyapatite, Behcet's syndrome, Felt's syndrome, hepatitis B, primary Sjoegrens, and polychondritis.

In another aspect of the invention, joint disease can also be the result of genetics, trauma (e.g., meniscus tears), mechanical injury (e.g., repetitive motion), nutrition deficiencies, and joint mal-alignment. Joints having suffered from an initial injury and/or trauma often develop joint disease over a period of time.

Examples of tissue diseases (e.g. epithelial, connective, muscle and nervous tissue) potentially treatable with the complex, compositions, and methods include, but are not limited to the following: autoimmune, degenerative, inflammatory, infectious, cancerous, viral, fungal, injured or trauma derived. These tissue and/or organ diseases may be the primary disease or may be caused by an existing disease and/or illness. Examples include amyloidosis, atiral fibrillation, convulsion, cramp, dermatomyositis, enchondroma, fibroma, lumbao, heritable connective tissue disorder (e.g., Marfan syndrome, Peyronie's disease, Ehlers-Danlos syndrome, Osteogenesis imperfecta, Stickler syndrome, Alport syndrome, Congenital contractural arachnodactyly), autoimmune connective tissue disorder (e.g., systemic lupus erythematosus (SLE), rheumatoid arthritis, Scleroderma, Sjoegren's syndrome, mixed connective tissue disease, psoriatic arthritis), scurvy, muscle disease (e.g., muscle tumour, muscular dystrophy, disuse atrophy, denervation atrophy, Duchenne muscular dystrophy, facioscapulohumoral muscular dystrophy), hepatic diseasemyasthenia gravis, myopathy, myositis, myositis ossificans, cancer, fibromyalgia, muscle fatigue, spasm, spasticity, sprain, strain, brain injury, spinal cord injury, gliomas, neuroeptheliomatous, hypertension, cardiovascular disease, diabetes, Alzheimer's disease, cystitis, AIDS, rickets, and nerve sheath tumors. Examples of tissues, organs and/or body systems affected by disease and may be treated with the compositions, and methods described therein, but are not limited to the following: Immune system, senory organs (e.g., organs of tase, smell, sight, hearing), digestive system (e.g., mouth, fauces, pharynx, esophagus, abdomen, stomach, small intestine, large intestine, liver, pancreas), urogenital apparatus, endocrinological systemt, metabolism, cardiovascular system (e.g., heart, blood pressure, arteries), hematology (e.g., blood chemistry), urinary organs (e.g., kidneys, ureters, urinary bladder, male urethra, female urethra, male gential organs (e.g., testes and their covering, ductus deferens, vesiculae seminales, ejaculatory ducts, penis, prostate, bulbourethral glands), female genital organs (e.g., ovaries, uterine tube, uterus, vagina, clitoris, Bartholin's glands, external organs, mammae)), ductless glands (e.g., thyroid, parathyroid, thymus, hypophysis cerebri, pineal body, chromaphil and corticol systems, spleen), reproduction, respiratory (e.g., larynx, trachea, bonchi, pleurae, mediastinum, lungs), central nervous system (e.g., nerves, nerve fibers), skin, epithelial (e.g., simple, stratified, pseudostratified columnar, glandular), connective (e.g., loose connective (e.g., areolar, adipose, reticular), and dense connective (e.g., dense regular, dense irregular)), cartilage (e.g., Hyaline, elastic, fibrous), muscle (e.g., skeletal muscle (e.g., type I, II, IIa, IN, IIb), cardiac muscle, smooth muscle), nervous (e.g., neuron (e.g., motor neurons, interneuron, sensory neuron), neuroglia, spinal cord, nerves, brain).

In another aspect of the invention, cancers can also reside in the joint, tissue and/or organ either as a primary tumor (e.g., sarcoma, hemangiopericytoma, connective tissue neoplasm, chondroma, chondrosarcoma) or as a result of metastasis of a primary tumor at a different location in the body of the subject.

Ex vivo and in vivo gene therapy with siRNA can also be used in joint, tissue, and/or organ disease. These RNAi applications toward joint disease include, but are not limited to, 1) targeting proteins or enzymes relevant in the disease state; 2) targeting or reducing expression of factors that are relevant in the disease state; and 3) targeting genes to maintain or restore joint health and homeostasis. For example, genes of the current invention may include ADAMTS (e.g., ADAMTS-4, ADAMTS-5), MMPs (e.g., MMP-1, MMP-3, MMP-9, MMP-13 and other MMPs), ILs (e.g., IL-1α, IL-1β, IL-2, IL-6, IL-8, IL-12, IL-15, IL-20, IL-21 and other ILs), IL receptors, IL receptor associated proteins, IL receptor antagonists, HLA-DRB1, PADI4, PTPN22, TNFAIP3, megakaryocyte stimulating factor, osteoprotegerin, activator of NF-α ligand, STAT4, CCR6, TNFR-1, TNFR-2, RIP, TRADD, PAD2-PAD4, FOX3, CD-25, FAP, DPP, CD26, MK2, SIRT-1, FoxO3a, miR-24, miR-125-5p, muR-203, miR-140, miR-365, miR-146a, miR-27a, TNF-α, HLA, collagen type II, aggrecan, prostaglandins, immunoglobulins, IFN-γ, GM-CSF, PDGF, FGF, VEGF, BMPs (e.g., BMP-2, BMP-4, BMP-7, and other BMPs), TGF-β, IGF-1, IGF-2 and, their related receptor protein and the like. For example, the following genes or proteins may promote arthritis such as rheumatoid arthritis: ADAMTS, MMPs, ILs, IL receptors, IL receptor associated proteins, HLA, DRB1, PADI4 gene, PTPN22 gene, TNFAIP3 gene, STAT4 gene, TNFR-1, TNFR-2, RIP, TRADD, PAD2-PAD4 proteins, CCR6 gene, miR-24, miR-125a-5p, mIR-365, miR-203, and miR-181a. In embodiments, the anti-miR-181a nucleic acid sequence comprises SEQ ID NO: 228 or SEQ ID NO: 229. Genes and protein can also prevent arthritis such as Juvenile idiopathic arthritis: FOXP3 and CD-25. Moreover, genes and proteins and their receptors and combinations thereof can also inhibit arthritis such as rheumatoid arthritis or osteoarthritis: IL receptor antagonists, MK2, FAP, DPP-4/CD26, SIRT-1/FoxO3a, miR-140 and miR-27a. Lastly, genes and proteins and their receptors and combinations thereof can mediate arthritis progression and joint tissue regeneration (such as cartilage regeneration): FGF, VEGF, BMPs, TGF-β, IGF-1, IGF-2, miR-146a.

Nanopieces deliver siRNA, antisense and/or anti-microRNA to knockdown genes and their related proteins and protein receptors (e.g., ADAMTS, MMPs, IL-1). In some embodiments, the anti-microRNA is anti-miR-24, anti-miR-125a-5p, anti-mIR-365, anti-miR-203, or anti-miR-181a. In an example, the anti-miR-181a nucleic acid sequence comprises SEQ ID NO: 228 or SEQ ID NO: 229. In another example, Nanopieces deliver miRNA and/or mRNA to increase the level of genes and their related proteins and protein receptors. For example, genes and expression their respective encoded proteins and/or corresponding protein receptors that promote arthritis or other joint diseases can be knocked down; while genes and expression of their encoded proteins and/or corresponding protein receptors that inhibit arthritis or other joint diseases can be increased. Gene expression and production of encoded proteins and/or corresponding protein receptors that mediate arthritis progression and joint tissue regeneration can be adjusted (either knocked down or increased) depending on the needs or clinical condition of the patient.

Ex vivo and in vivo gene therapy with siRNA could also be used in cancer of tissue and/or organs. These RNAi applications toward cancer include, but are not limited to, 1) reducing expression of growth factors, reducing proteins that augment the cell cycle (e.g., Raf-1, PI-3 kinase), growth factor receptors (e.g., EGFR, Her-2), or proteins critical for supporting cells of the tumor (e.g., VEGF, VEGFR1-2 for tumor endothelial cells); 2) targeting or reducing expression of factors that are anti-apoptotic (e.g., BCL-2); and 3) targeting proteins or enzymes that reduce immune activation toward tumor.

Cancers or neoplasms contemplated within the scope of the disclosure include, but are not limited to, carcinomas (i.e., malignant tumors derived from epithelial cells such as, for example, common forms of breast, prostate, lung and colon cancer), sarcomas (i.e., malignant tumors derived from connective tissue or mesenchymal cells), lymphomas (i.e., malignancies derived from hematopoietic cells), leukemias (i.e., malignancies derived from hematopoietic cells), germ cell tumors (i.e., tumors derived from totipotent cells. In adults most often found in the testicle or ovary; in fetuses, babies and young children, most often found on the body midline, particularly at the tip of the tailbone), blastic tumors (i.e., a typically malignant tumor which resembles an immature or embryonic tissue) and the like.

Examples of specific neoplasms intended to be encompassed by the present invention include, but are not limited to, acute lymphoblastic leukemia, myeloid leukemia, acute childhood myeloid leukemia, adrenocortical carcinoma, AIDS-related cancers, AIDS-related lymphoma, anal cancer, appendix cancer, astrocytoma (e.g., cerebellar, cerebral), atypical teratoid/rhabdoid tumor, basal cell carcinoma, extrahepatic bile duct cancer, bladder cancer, bone cancer, osteosarcoma and malignant fibrous histiocytoma, brain tumor (e.g., brain stem glioma, central nervous system atypical teratoid/rhabdoid tumors, central nervous system embryonal tumors, cerebellar astrocytoma, cerebral astrocytoma/malignant glioma, craniopharyngioma, ependymoblastoma, ependymoma, medulloblastoma, medulloepithelioma, pineal parenchymal tumors of intermediate differentiation, supratentorial primitive neuroectodermal tumors and/or pineoblastoma, visual pathway and/or hypothalamic glioma, brain and spinal cord tumors), breast cancer, bronchial tumors, Burkitt lymphoma, carcinoid tumor (e.g., gastrointestinal), carcinoma of unknown primary, central nervous system (e.g., atypical teratoid/rhabdoid tumor, embryonal tumors (e.g., lymphoma, primary), cerebellar astrocytoma, cerebral astrocytoma/malignant glioma, cervical cancer, chordoma, chronic lymphocytic leukemia, chronic myelogenous leukemia, chronic myeloproliferative disorders, colon cancer, colorectal cancer, craniopharyngioma, cutaneous T-cell lymphoma, central nervous system embryonal tumors, endometrial cancer, ependymoblastoma, ependymoma, esophageal cancer, Ewing family of tumors, extracranial germ cell tumor, extragonadal germ cell tumor, extrahepatic bile duct cancer, eye cancer (e.g., intraocular melanoma, retinoblastoma), gallbladder cancer, gastric cancer, gastrointestinal tumor (e.g., carcinoid tumor, stromal tumor (gist), stromal cell tumor), germ cell tumor (e.g., extracranial, extragonadal, ovarian), gestational trophoblastic tumor, glioma (e.g., brain stem, cerebral astrocytoma), hairy cell leukemia, head and neck cancer, hepatocellular cancer, Hodgkin lymphoma, hypopharyngeal cancer, hypothalamic and visual pathway glioma, intraocular melanoma, islet cell tumors, Kaposi sarcoma, kidney cancer, large cell tumors, laryngeal cancer (e.g., acute lymphoblastic, acute myeloid), leukemia (e.g., acute myeloid, chronic lymphocytic, chronic myelogenous, hairy cell), lip and/or oral cavity cancer, liver cancer, lung cancer (e.g., non-small cell, small cell), lymphoma (e.g., AIDS-related, Burkitt, cutaneous T cell, Hodgkin, non-Hodgkin, primary central nervous system), Waldenström macroglobulinemia, malignant fibrous histiocytoma of bone and/or osteosarcoma, medulloblastoma, medulloepithelioma, melanoma, merkel cell carcinoma, mesothelioma, metastatic squamous neck cancer, mouth cancer, multiple endocrine neoplasia syndrome, multiple myeloma/plasma cell neoplasm, mycosis fungoides, myelodysplastic syndromes, myelodysplastic/myeloproliferative diseases, myelogenous leukemia (e.g., chronic, acute, multiple), chronic myeloproliferative disorders, nasal cavity and/or paranasal sinus cancer, nasopharyngeal cancer, neuroblastoma, non-Hodgkin lymphoma, non-small cell lung cancer, oral cancer, oral cavity cancer, oropharyngeal cancer, osteosarcoma and/or malignant fibrous histiocytoma of bone, ovarian cancer (e.g., ovarian epithelial cancer, ovarian germ cell tumor, ovarian low malignant potential tumor), pancreatic cancer (e.g., islet cell tumors), papillomatosis, paranasal sinus and/or nasal cavity cancer, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, plasma cell neoplasm/multiple myeloma, pleuropulmonary blastoma, primary central nervous system lymphoma, prostate cancer, rectal cancer, renal cell cancer, renal, pelvis and/or ureter, transitional cell cancer, respiratory tract carcinoma involving the nut gene on chromosome 15, retinoblastoma, rhabdomyosarcoma, salivary gland cancer, sarcoma (e.g., Ewing family of tumors, Kaposi, soft tissue, uterine), Sézary syndrome, skin cancer (e.g., non-melanoma, melanoma, merkel cell), small cell lung cancer, small intestine cancer, soft tissue sarcoma, squamous cell carcinoma, squamous neck cancer with occult primary, metastatic, stomach cancer; supratentorial primitive neuroectodermal tumors; T-cell lymphoma, cutaneous; testicular cancer, throat cancer; thymoma and/or thymic carcinoma, thyroid cancer, transitional cell cancer of the renal, pelvis and/or ureter; trophoblastic tumor, unknown primary site carcinoma, urethral cancer, uterine cancer, endometrial, uterine sarcoma, vaginal cancer, visual pathway and/or hypothalamic glioma, vulvar cancer, Waldenström macroglobulinemia, Wilms tumor and the like. For a review, see the National Cancer Institute's Worldwide Website (cancer.gov/cancertopics/alphalist). One of skill in the art will understand that this list is exemplary only and is not exhaustive, as one of skill in the art will readily be able to identify additional cancers and/or neoplasms based on the disclosure herein.

Examples of primary cancers as joint disease comprise connective tissue neoplasm, hemangiopericytoma, sarcoma, chondroma, chondrosarcoma, bone and the like.

Examples of genetic and/or non-neoplastic diseases potentially treatable with the complex, compositions, and methods include, but are not limited to the following: adenosine deaminase deficiency; purine nucleoside phosphorylase deficiency; chronic granulomatous disease with defective p47phox; sickle cell with HbS, β-thalassemia; Faconi's anemia; familial hypercholesterolemia; phenylketonuria; ornithine transcarbamylase deficiency; apolipoprotein E deficiency; hemophilia A and B; muscular dystrophy; cystic fibrosis; Parkinsons, retinitis pigmentosa, lysosomal storage disease (e.g., mucopolysaccharide type 1, Hunter, Hurler and Gaucher), diabetic retinopathy, human immunodeficiency virus disease virus infection, acquired anemia, cardiac and peripheral vascular disease, osteoporosis and arthritis. In some of these examples of diseases, the therapeutic gene may encode a replacement enzyme or protein of the genetic or acquired disease, an antisense or ribozyme molecule, a decoy molecule, or a suicide gene product.

Recombinant cells may be produced using the complexes of the present invention. Resulting recombinant cells can be delivered to a subject by various methods known in the art. In certain embodiments, the recombinant cells are injected, e.g., subcutaneously or intra-articular. Recombinant blood cells (e.g., hematopoietic stem or progenitor cells) are preferably administered intravenously or intra-articular. The cells can also be encapsulated in a suitable vehicle and then implanted in the subject (see, e.g., Dionne et al. PCT Publication WO92/19195, dated Nov. 12, 1992). The amount of cells administered depends on a variety of factors known in the art, for example, the desired effect, subject state, rate of expression of the chimeric polypeptides, etc., and can readily be determined by one skilled in the art.

Another aspect of the present disclosure provides methods of introducing a therapeutic or diagnostic agent into a cell or tissue matrix using rosette nanotubes. Biologically active agents also called “therapeutic agents” or “drugs” are complexed with rosette nanotubes to form nanotube-drug complex, which can enter the cell and/or tissue and release the drug. A person of skill in the art will recognize the drug as being compounds which include any synthetic or natural element or are compounds which when introduced into the body causes a desired biological response, such as altering body function. Non-limiting examples of drugs or biologically active agents or therapeutic agents include anti-inflammatory agents (e.g., steroidal and non-steroidal), analgesics, anesthetics, chemotherapeutic agents, anti-proliferative agents, cytotoxic agents, steroidal agents, antifungal agents, antiviral agents, immunosuppressive agents, and include small molecules. Further non-limiting examples of drugs or biologically active agents or therapeutic agents include peptides (such as RGD, KRSR (SEQ ID NO: 232), YIGSR (SEQ ID NO: 233), IKVAV (SEQ ID NO: 234) and the like), aromatic bioactive molecules such as tamoxifen, dexamethasone, vitamin K and the like, antibiotics such as penicillin, streptomycin, gentamycin and the like, glucosamine, chondroitin, cortisone, glucocorticoids, hydrocortisone, hyaluronic acid, hydrocortisone, gentamycin and the like, and proteins such as bone morphogenetic proteins, matrillins and the like. Drugs or biologically active agents or therapeutic agents may be hydrophobic or hydrophilic. According to one aspect, the rosette nanotubes include hydrophobic moieties within the core portion of the structure where hydrophobic drugs, biologically active agents or therapeutic agents may be located in the composite. According to another aspect, the rosette nanotubes of the present disclosure may have hydrophilic outer surfaces to facilitate administration of the complexes in physiological environments.

Examples of analgesic agents include opioid analgesics and adjuvent analgesics within the scope of the present disclosure that can be complexed with rosette nanotubes include clonidine, tizanidine, gapapentin, pregabalin, lamotrigine, oxcarbazepine, topiramate, levitiracetam, tigabine, zonisamide, carbamazepine, valprioc acid, phenytoin, amitriptyline, nortriptyline, desipramine, imipramine, doxepin, paroxetine, citalopram, escitalopram, fluoxetine, venlafaxine, duloxetine, bupriopion, mexiletine, lidocaine, baclofen, cyclobenzaprine, orphenadrine, metaxalone, methocarbamol, morphine, hydrocodone, hydromorphone, tramadol, oxycodone, oxymorphone, fentanyl, methadone, capsaicin, loperamide, naloxone, demerol, buprenorphine, butorphanol, codeine, levorphanol, meperidine, methadone, nabuphine, propoxyphene, and pentazocine.

Examples of non-opioid and anti-inflammatory agents within the scope of the present disclosure that can be complexed with rosette nanotubes include acetaminophen, aspirin, diflunisal, choline magnesium trisalicylate, salsalate, ibuprofen, naproxen, ketoprofen, fluriprofen, oxaprozin, indomethacin, sulindac, nabumetone, diclofenac, ketorolac, tolectin, piroxicam, meloxicam, mefenamic acid, meclofenamate, celecoxib, allopurinol, dextromethorphan, pegloticase, dexibuprofen, etodolac, fenoprofen, flufenamic acid, flupbiprofen, lornoxicam, loxoprofen, meclofenamic acid, piroxicam, tenoxicam, tolmetin, and tolfenamic acid.

Examples of immunosuppresive agents within the scope of the present disclosure that can be complexed with rosette nanotubes include alkylating agents, antimetabolites, high dose corticosteroids, azathioprine, mycophenolate mofetil, cyclosporine, methotrexate, leflunomide, cyclophosphamide, chlorambucil, nitrogen mustard, abacavir, abciximab, adalimumab, aldesleukin, altretamine, aminoglutethimide, amprevenir, anakinra, anastrozole, aspariginase, azathioprine, basiliximab, betamethasone, bleomycin, busulfan, capecitabine, carboplatin, carmustine, chlorambucil, cidofovir, cisplatin, cladribine, cortisone, cyclosporine, cytarabine, decarbazine, dacuzumab, dactinomycin, daunorubicin, delaviridine, dexamethasone, didanosine, doxorubicin, efavirenz, epirubicin, estramustine, etanercept, etoposide, exemestane, foxuridine, fludarabine, fluorouracil, flutamide, gemcitabine, gemtuzumab ozogamicin, hydrocortisone, hydroxychloroquine, hydroxyurea, idaubicin, ifosphamide, indinavir, infliximab, interferon alpha-2a, interferon alpha-2b, interferon beta-2b, interferon beta-2a, interferon gamma-1b, interleukin-2, irinotecan, isotretinoin, lamivudine, leflunomide, letrozole, leuprolide, mechloethamine, megestrol, melphalan, mercaptopurine, methotrexate, methylpregnisolone, mitomycin, mitotane, mitoxantrone, mycophenolate, nelfinavir, nevirapine, paclitaxel, pegaspargase, penicillamine, pentostatin, pimecroslimus, pipobroman, plicamycin, prednisolone, predisone, priliximab, procarbazine, ritonavir, rituximab, saquinavir, sargamomstim, stavudine, strepozocin, tacrolismus, temozolomide, teniposide, testolactone, thioguanine, thiotepa, trastuzumab, tretinoin, triamcinolone, uracil mustard, valrubucin, vinblastine, vincristine, vinorelbine, zalcitabine, zidovudine.

Examples of antifungal agents within the scope of the present disclosure that can be complexed with rosette nanotubes include polyene, azole, allylamine, morpholine, and antimetabolite antifungal agents, e.g., amphotericin B, candicin, filipin, hamycin, natamycin, nystatin rimocidin, bifonazole, butoconazole, clotrimazole, econozole, fenticonazole, isoconazole, ketoconazole, luiconazole, miconazole, omoconazole, oxiconazole, sertaconazole, sulconazole, tioconazole, albaconazole, fluconazole, isavuconazole, traconazole, posaconazole, ravuconazole, terconazole, voriconazole, abafungin, amorolfin, butenafine, naftifine, terbinafine, anidulafungin, caspofungin, micafungin, benzoic acid, ciclopirox, griseofulvin, tolnaftate, and undecylenic acid.

Examples of antibiotic agent within the scope of the present disclosure that can be complexed with rosette nanotubes include aminoglycosides (e.g., amikacin, gentamicin, kanamycine, neomycine, metilmicin, tobramycin, paromomycin, streptomycin, spectinomycin), anasamycins (e.g., geldanamycin, herbimycin, riflaximin), loracerbef, carbapenems (e.g., ertapenem, doripenem, cilastatin, meropenem), cephalosporin (e.g. cefadroxil, cefazolin, cephalexin, cefaclor, cefamandole, cefoxitin, cefprozil, cefuroxime, cefixime, cefdinir, cefdotoren, cefotaxime, ceftibuten, ceftizoxime, cefepime, ceftaroline, ceftobioprole, teichoplanin, vancomycin, telavancin, clindamycin, lincomycin, daptomycin, azithromycin, clarithromycin, dirithromycin, erythromycin, roxithromycin, troleandomycin, telithromycin, spiramycin, azetreonam, flurazolidone, linezolid, posizolid, radezolid, torezolid, ampicillin, azolocillin, carbenicillin, cloxacillin, dicloxaxillin, pencillin), polypeptides (e.g. bacitracin, colistin, polymyxin B), Quinolones (e.g., ciproflaxin, enoxacin, gemifloxacin, norfloxacin), sulfonamides (e.g., malfenide, sulfamethizole, sulfasalazine, sulfadiazine), tetracyclines (e.g., demeclocycline, minocycline, doxycycline, tetracycline), clofazimine, dapsone, capreomycin, cycloserine, ethambutol, ethionamide, isoniazid, pyrazinamide, riflampicin, rifabutin, rifapentine, streptomycin, arsphenamine, chloramthenicol, foffmycin, fusidic acid, metronidazole, mupirocin, platensimycin, thiamphenicol, tigecycline, tinidazole, and trimethoprim.

Examples of drugs within the scope of the present disclosure that can be complexed with rosette nanotubes include glucosamine, chondroitin, cortisone, glucocorticoids, hydrocortisone, hyaluronic acid, hydrocortisone, and lurbicants (e.g. lubricin).

Examples of anti-cancer drugs within the scope of the present disclosure that can be complexed with rosette nanotubes include bortezomib ([(1R)-3-methyl-1-[[(2S)-1-oxo-3-phenyl-2-[(pyrazinylcarbonyl) amino]propyl]amino]butyl] boronic acid; MG-341; VELCADE®), MG-132 (N-[(phenylmethoxy)carbonyl]-L-leucyl-N-[(1S)-1-formyl-3-methylbutyl]-leucinamide); pyrimidine analogs (e.g., 5-fluorouracil, floxuridine, capecitabine, gemcitabine and cytarabine); purine analogs; folate antagonists and related inhibitors (e.g., mercaptopurine, thioguanine, pentostatin and 2-chlorodeoxyadenosine [cladribine]); folic acid analogs (e.g., methotrexate); antimitotic agents, including vinca alkaloids (e.g., vinblastine, vincristine, and vinorelbine) and alkylating agents such as nitrogen mustards (e.g., mechlorethamine, cyclophosphamide and analogs, melphalan, chlorambucil), ethylenimines and methylmelamines (e.g., hexamethylmelamine and thiotepa), alkyl sulfonates-busulfan, nitrosoureas (e.g., carmustine (BCNU) and analogs, streptozocin), trazenes-dacarbazinine (DTIC); microtubule disruptors (e.g., paclitaxel, docetaxel, vincristin, vinblastin, nocodazole, epothilones and navelbine, and teniposide); actinomycin, amsacrine, anthracyclines, bleomycin, busulfan, camptothecin, carboplatin, chlorambucil, cisplatin, cyclophosphamide, Cytoxan, dactinomycin, daunorubicin, docetaxel, doxorubicin, epirubicin, hexamethylmelamineoxaliplatin, iphosphamide, melphalan, merchlorethamine, mitomycin, mitoxantrone, nitrosourea, paclitaxel, plicamycin, procarbazine, teniposide, triethylenethiophosphoramide and etoposide (VP 16); dactinomycin (actinomycin D), daunorubicin, doxorubicin (adriamycin), idarubicin, anthracyclines, mitoxantrone, bleomycins, plicamycin (mithramycin) and mitomycin; L-asparaginase; antiplatelet agents; platinum coordination complexes (e.g., cisplatin, carboplatin), procarbazine, hydroxyurea, mitotane, aminoglutethimide; hormones and hormone analogs (e.g., estrogen, tamoxifen, goserelin, bicalutamide, nilutamide); aromatase inhibitors (e.g., letrozole, anastrozole); anticoagulants (e.g., heparin, synthetic heparin salts and other inhibitors of thrombin); fibrinolytic agents (such as tissue plasminogen activator, streptokinase and urokinase), aspirin, COX-2 inhibitors, dipyridamole, ticlopidine, clopidogrel, abciximab; antimigratory agents; antisecretory agents (e.g., breveldin); immunosuppressives (e.g., cyclosporine, tacrolimus (FK-506), sirolimus (rapamycin), azathioprine, mycophenolate mofetil); anti-angiogenic compounds (e.g., TNP-470, genistein) and growth factor inhibitors (e.g., vascular endothelial growth factor (VEGF) inhibitors, fibroblast growth factor (FGF) inhibitors, epidermal growth factor (EGF) inhibitors); angiotensin receptor blockers; nitric oxide donors; anti-sense oligonucleotides; antibodies (e.g., trastuzumab (HERCEPTIN®), AVASTIN®, ERBITUX®); cell cycle inhibitors and differentiation inducers (e.g., tretinoin); mTOR (mammalian target of rapamycin) inhibitors (e.g., everolimus, sirolimus); topoisomerase inhibitors e.g., doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan); corticosteroids (e.g., cortisone, dexamethasone, hydrocortisone, methylpednisolone, prednisone, and prenisolone); growth factor signal transduction kinase inhibitors; mitochondrial dysfunction inducers; and caspase activators and the like.

Examples of anti-cancer drugs within the scope of the present disclosure that can be complexed with rosette nanotubes include alemtuzumab; aminoglutethimide; amsacrine; anastrozole; asparaginase; bevacizumab; bicalutamide; bleomycin; bortezomib; buserelin; busulfan; campothecin; capecitabine; carboplatin; carmustine; CeaVac; cetuximab; chlorambucil; cisplatin; cladribine; clodronate; colchicine; cyclophosphamide; cyproterone; cytarabine; dacarbazine; daclizumab; dactinomycin; daunorubicin; dienestrol; diethylstilbestrol; docetaxel; doxorubicin; edrecolomab; epirubicin; epratuzumab; erlotinib; estradiol; estramustine; etoposide; exemestane; filgrastim; fludarabine; fludrocortisone; fluorouracil; fluoxymesterone; flutamide; gemcitabine; gemtuzumab; genistein; goserelin; huJ591; hydroxyurea; ibritumomab; idarubicin; ifosfamide; IGN-101; imatinib; interferon; irinotecan; ironotecan; letrozole; leucovorin; leuprolide; levamisole; lintuzumab; lomustine; MDX-210; mechlorethamine; medroxyprogesterone; megestrol; melphalan; mercaptopurine; mesna; methotrexate; mitomycin; mitotane; mitoxantrone; mitumomab; nilutamide; nocodazole; octreotide; oxaliplatin; paclitaxel; pamidronate; pentostatin; pertuzumab; plicamycin; porfimer; procarbazine; raltitrexed; rituximab; streptozocin; sunitinib; suramin; tamoxifen; temozolomide; teniposide; testosterone; thalidomide; thioguanine; thiotepa; titanocene dichloride; topotecan; tositumomab; trastuzumab; tretinoin; vatalanib; vinblastine; vincristine; vindesine; and vinorelbine and the like.

Examples of NMDA receptor antagonists within the scope of the present disclosure that can be complexed with rosette nanotubes include LY 274614 (decahydro-6-(phosphonomethyl)-3-isoquinolinecarboxylic acid), LY 235959 [(3S,4aR,6S,8aR)-decahydro-6-(phosphonomethyl)-3-isoquinolinecarboxylic acid], LY 233053 ((2R,4S)-rel-4-(1H-tetrazol-5-yl-methyl)-2-piperidine carboxylic acid), NPC 12626 (α-amino-2-(2-phosphonoethyl)-cyclohexanepropanoic acid), reduced and oxidized glutathione, carbamathione, AP-5 (5-phosphono-norvaline), CPP (4-(3-phosphonopropyl)-2-piperazine-carboxylic acid), CGS-19755 (seifotel, cis-4(phono-methyl)-2-piperidine-carboxylic acid), CGP-37849 ((3E)-2-amino-4-methyl-5-phosphono-3-pentenoic acid), CGP 39551 ((3E)-2-amino-4-methyl-5-phosphono-3-pentenoic acid, 1-ethyl ester), SDZ 220-581 [(αS)-α-amino-2′-chloro-5-(phosphonomethyl)-[1,1′-biphenyl]-3-propanoic acid], and S-nitrosoglutathione. amantadine, aptiganel (CERESTAT®, CNS 1102), caroverine, dextrorphan, dextromethorphan, fullerenes, ibogaine, ketamine, lidocaine, memantine, dizocilpine (MK-801), neramexane (MRZ 2/579, 1,3,3,5,5-pentamethyl-cyclohexanamine), NPS 1506 (delucemine, 3-fluoro-γ-(3-fluorophenyl)-N-methyl-benzenepropanamine hydrochloride), phencyclidine, tiletamine and remacemide. acamprosate, arcaine, conantokin-G, eliprodil (SL 82-0715), haloperidol, ifenprodil, traxoprodil (CP-101,606), and Ro 25-6981 [(±)—(R,S)-α-(4-hydroxyphenyl)-O-methyl-4-(phenylmethyl)-1-piperidine propanol]; aminocyclopropanecarboxylic acid (ACPC), 7-chlorokynurenic acid, D-cycloserine, gavestinel (GV-150526), GV-196771A (4,6-dichloro-3-[(E)-(2-oxo-1-phenyl-3-pyrrolidinylidene)methyl]-1H-indole-2-carboxylic acid monosodium salt), licostinel (ACEA 1021), MRZ-2/576 (8-chloro-2,3-dihydropyridazino[4,5-b]quinoline-1,4-dione 5-oxide 2-hydroxy-N,N,N-trimethyl-ethanaminium salt), L-701,324 (7-chloro-4-hydroxy-3-(3-phenoxyphenyl)-2(1H)-quinolinone), HA-966 (3-amino-1-hydroxy-2-pyrrolidinone), and ZD-9379 (7-chloro-4-hydroxy-2-(4-methoxy-2-methylphenyl)-1,2,5,10-tetra-hydropyridanizo[4,5-b]quinoline-1,10-dione, sodium salt); oxidized and reduced glutathione, S-nitrosoglutathione, sodium nitroprusside, ebselen, and disulfiram, DETC-MeSO, carbamathione; CNQX (1,2,3,4-tetrahydro-7-nitro-2,3-dioxo-6-quinoxalinecarbonitrile) and DNQX (1,4-dihydro-6,7-dinitro-2,3-quinoxalinedione) and the like.

Examples of subtype-specific NMDA receptor antagonists within the scope of the present disclosure that can be complexed with rosette nanotubes include arcaine, argiotoxin636, Co 101244 (PD 174494, Ro 63-1908, 1-[2-(4-hydroxyphenoxy)ethyl]-4-[(4-methylphenyl)methyl-4-piperidinol], despiramine, dextromethorphan, dextrorphan, eliprodil, haloperidol, ifenprodil, memantine, philanthotoxin343, Ro-25-6981 ([(±)—(R*, S*)-α-(4-hydroxyphenyl)-β-methyl-4-(phenylmethyl)-1-piperidine propanol]), traxoprodil (CP-101,606), Ro 04-5595 (1-[2-(4-chlorophenyl)ethyl]-1,2,3,4-tetrahydro-6-methoxy-2-methyl-7-isoquinolinol), CPP [4-(3-phosphonopropyl)-2-piperazinecarboxylic acid], conantokin G, spermine, spermidine, NVP-AAM077 [[[[(1S)-1-(4-bromophenyl)ethyl]amino](1,2,3,4-tetrahydro-2,3-dioxo-5-quinoxalinyl)methyl]-phosphonic acid]; and 1-(phenanthrene-2-carbonyl) piperazine-2,3-dicarboxylic acid and the like.

Examples of anticonvulsants within the scope of the present disclosure that can be complexed with rosette nanotubes include barbiturates (e.g., mephobarbital and sodium pentobarbital); benzodiazepines, such as alprazolam (XANAX®), lorazepam, clonazepam, clorazepate dipotassium, and diazepam (VALIUM®); GABA analogs, such as tiagabine, gabapentin (an α2δ antagonist, NEURONTIN®), and β-hydroxypropionic acid; hydantoins, such as 5,5-diphenyl-2,4-imidazolidinedione (phenytoin, DILANTIN®) and fosphenytoin sodium; phenyltriazines, such as lamotrigine; succinimides, such as methsuximide and ethosuximide; 5H-dibenzazepine-5-carboxamide (carbamazepine); oxcarbazepine; divalproex sodium; felbamate, levetiracetam, primidone; zonisamide; topiramate; and sodium valproate.

Examples of psychiatric drugs within the scope of the present disclosure that can be complexed with rosette nanotubes include Abilify, Adapin, Adartrel, Adderall, Alepam, Alertec, Aloperidin, Alplax, Alprax, Alprazolam, Alviz, Alzolam, Amantadine, Ambien, Amisulpride, Amitriptyline, Amoxapine, Amfebutamone, Anafranil, Anatensol, Ansial, Ansiced, Antabus, Antabuse, Antideprin, Anxiron, Apo-Alpraz, Apo-Primidone, Apo-Sertral, Aponal, Apozepam, Aripiprazole, Aropax, Artane, Asendin, Asendis, Asentra, Ativan, Atomoxetine, Aurorix, Aventyl, Axoren, Beneficat, Benperidol, Bimaran, Bioperidolo, Biston, Brotopon, Bespar, Bupropion, Buspar, Buspimen, Buspinol, Buspirone, Buspisal, Cabaser, Cabergoline, Calepsin, Calcium carbonate, Calcium carbimide, Calmax, Carbamazepine, Carbatrol, Carbolith, Celexa, Chloraldurat, Chloralhydrat, Chlordiazepoxide, Chlorpromazine, Cibalith-S, Cipralex, Citalopram, Clomipramine, Clonazepam, Clozapine, Clozaril, Concerta, Constan, Convulex, Cylert, Dapotum, Daquiran, Daytrana, Defanyl, Dalmane, Damixane, Demolox, Depad, Depakene, Depakote, Depixol, Desyrel, Dostinex, dextroamphetamine, Dexedrine, Diazepam, Didrex, Divalproex, Dogmatyl, Dolophine, Droperidol, Edronax, Efectin, Effexor (Efexor), Eglonyl, Einalon S, Elavil, Elontril, Endep, Epanutin, Epitol, Equetro, Escitalopram, Eskalith, Eskazinyl, Eskazine, Etrafon, Eukystol, Eunerpan, Faverin, Fazaclo, Fevarin, Finlepsin, Fludecate, Flunanthate, Fluoxetine, Fluphenazine, Flurazepam, Fluspi, Fluspirilen, Fluvoxamine, Focalin, Gabapentin, Geodon, Gladem, Glianimon, Halcion, Halomonth, Haldol, Haloperidol, Halosten, Imap, Imipramine, Imovane, JJanimine, Jatroneural, Kalma, Keselan, Klonopin, Lamotrigine, Largactil, Lecital, Levomepromazine, Levoprome, Leponex, Lexapro, Libritabs, Librium, Linton, Liskantin, Lithane, Lithium, Lithizine, Lithobid, Lithonate, Lithotabs, Lorazepam, Loxapac, Loxapine, Loxitane, Ludiomil, Lunesta, Lustral, Luvox, Lyrica, Lyogen, Manegan, Manerix, Maprotiline, Mellaril, Melleretten, Melleril, Melneurin, Melperone, Meresa, Mesoridazine, Metadate, Methamphetamine, Methotrimeprazine, Methylin, Methylphenidate, Minitran, Mirapex, Mirapexine, Moclobemide, Modafinil, Modalina, Modecate, Moditen, Molipaxin, Moxadil, Murelax, Myidone, Mylepsinum, Mysoline, Nardil, Narol, Navane, Nefazodone, Neoperidol, Neurontin, Nipolept, Norebox, Normison, Norpramine, Nortriptyline, Novodorm, Olanzapine, Omca, Oprymea, Orap, Oxazepam, Pamelor, Parnate, Paroxetine, Paxil, Peluces, Pemoline, Pergolide, Permax, Permitil, Perphenazine, Pertofrane, Phenelzine, Phenytoin, Pimozide, Piportil, Pipotiazine, Pragmarel, Pramipexole, Pregabalin, Primidone, Prolift, Prolixin, Promethazine, Prothipendyl, Protriptyline, Provigil, Prozac, Prysoline, Psymion, Quetiapine, Ralozam, Reboxetine, Resimatil, Restoril, Restyl, Requip, Rhotrimine, Risperdal, Risperidone, Rispolept, Ritalin, Rivotril, Ropark, Ropinerole, Rubifen, Rozerem, Sediten, Seduxen, Selecten, Serax, Serenace, Serepax, Serenase, Serentil, Seresta, Serlain, Serlift, Seroquel, Seroxat, Sertan, Sertraline, Serzone, Sevinol, Sideril, Sifrol, Sigaperidol, Sinequan, Sinqualone, Sinquan, Sirtal, Solanax, Solian, Solvex, Songar, Stazepin, Stelazine, Stilnox, Stimuloton, Strattera, Sulpiride, Sulpiride Ratiopharm, Sulpiride Neurazpharm, Surmontil, Symbyax, Symmetrel, Tafil, Tavor, Taxagon, Tegretol, Telesmin, Temazepam, Temesta, Temposil, Terfluzine, Thioridazine, Thiothixene, Thombran, Thorazine, Timonil, Tofranil, Tradon, Tramadol, Tramal, Trancin, Tranax, Trankimazin, Tranquinal, Tranylcypromine, Trazalon, Trazodone, Trazonil, Trialodine, Trevilor, Triazolam, Trifluoperazine, Trihexane, Trihexyphenidyl, Trilafon, Trimipramine, Triptil, Trittico, Troxal, Tryptanol, Ultram, Valium, Valproate, Valproic acid, Valrelease, Vasiprax, Venlafaxine, Vestra, Vigicer, Vivactil, Wellbutrin, Xanax, Xanor, Xydep, Zamhexal, Zeldox, Zimovane, Zispin, Ziprasidone, Zolarem, Zoldac, Zoloft, Zolpidem, Zonalon, Zopiclone, Zotepine, Zydis, Zyprexa and the like.

Examples of miscellaneous drugs within the scope of the present disclosure that can be complexed with rosette nanotubes include nortriptyline, amytriptyline, fluoxetine (PROZAC®), paroxetine HCl (PAXIL®), trimipramine, oxcarbazepine (TRILEPTAL®), eperisone, misoprostol (a prostaglandin E₁ analog), latanoprost (a prostaglandin F₂ [symbol] analog) melatonin, and steroids (e.g., pregnenolone, triamcinolone acetonide, methylprednisolone, and other anti-inflammatory steroids) and the like.

Examples of antiviral drugs within the scope of the present disclosure that can be complexed with rosette nanotubes include Abacavir, Aciclovir, Acyclovir, Adefovir, Amantadine, Amprenavir, Ampligen, Arbidol, Atazanavir, Atripla (fixed dose drug), Boceprevir, Cidofovir, Combivir (fixed dose drug), Darunavir, Delavirdine, Didanosine, Docosanol, Edoxudine, Efavirenz, Emtricitabine, Enfuvirtide, Entecavir, Entry inhibitors, Famciclovir, Fixed dose combination (antiretroviral), Fomivirsen, Fosamprenavir, Foscarnet, Fosfonet, Fusion inhibitor, Ganciclovir, Ibacitabine, Imunovir, Idoxuridine, Imiquimod, Indinavir, Inosine, Integrase inhibitor, Interferon type III, Interferon type II, Interferon type I, Interferon, Lamivudine, Lopinavir, Loviride, Maraviroc, Moroxydine, Methisazone, Nelfinavir, Nevirapine, Nexavir, Nucleoside analogues, Oseltamivir (Tamiflu), Peginterferon alfa-2a, Penciclovir, Peramivir, Pleconaril, Podophyllotoxin, Protease inhibitor (pharmacology), Raltegravir, Reverse transcriptase inhibitor, Ribavirin, Rimantadine, Ritonavir, Pyramidine, Saquinavir, Stavudine, Synergistic enhancer (antiretroviral), Tea tree oil, Tenofovir, Tenofovir disoproxil, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), Zidovudine and the like.

Ex vivo and in vivo therapy and/or diagnostics could also be used in joint disease. These therapeutic and diagnostic applications toward these joint diseases include, but are not limited to, 1) targeting proteins or enzymes relevant in the disease state; 2) targeting or reducing expression of factors that are relevant in the disease state; and 3) targeting genes to maintain or restore joint health and homeostasis. For example, Nanopieces delivery of molecular probes to detect expression of inflammatory markers (e.g., cytokines, MMP, ADAMS) and the like or delivery of therapeutic agents to treat pain, inflammation, infection and the like can be used.

In another example, in vivo imaging technology to detect molecular changes at early stages of arthritis without harming articular cartilage was demonstrated. Osteoarthritis (OA) is one of the most common causes of disability. However, the lack of tools for early diagnosis of OA hampers the prevention and treatment of the disease to decelerate articular cartilage loss and alleviate suffering of patients. The OA Biomarker Initiative has identified a series of biomarkers, including Matrix metalloproteinases (MMP), which are elevated in articular cartilage during OA pathogenesis. However, detection of MMP protein levels or activities in serum may not be sensitive enough, while the more sensitive detection of MMP transcripts requires invasive procedure to obtain biopsy of articular joint tissue. Therefore, there is an urgent need to develop sensitive in vivo imaging technology to detect molecular changes at early stages of arthritis without harming articular cartilage.

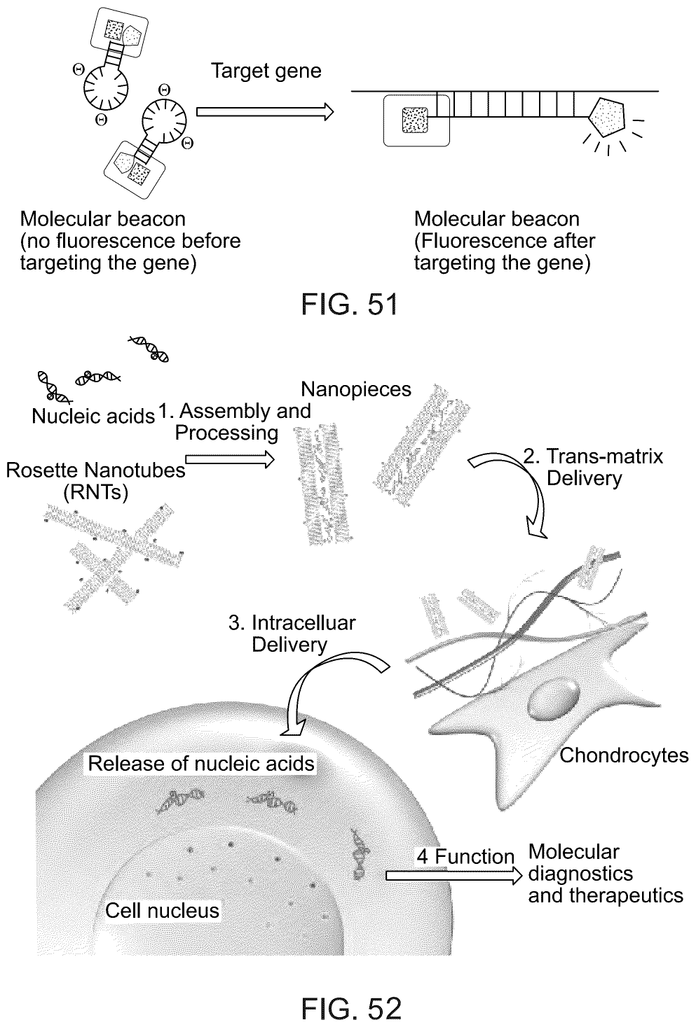

Specifically, Molecular beacon (MB) technology provided an intriguing possibility to detect the changes of mRNA levels in live animals in vivo. In fact, molecular beacon (MB) technology (FIG. 51) detected the changes of mRNA levels in live animals in vivo. The Molecular beacon comprises an oligonucleotides loop, double strand stem, and a fluorophore and quencher, which remains non-fluorescent due to the proximity of fluorophore and quencher. Upon entering a cell and hybridizing with its target mRNA, MB emits fluorescence after separation of the fluorophore and quencher (FIG. 52). However, prior to the invention, there was no report of detection of OA using MB due to the significant challenge of in vivo delivery of MB into joint tissues. Detection of OA using MB is challenging because of the in vivo delivery of MB into joint tissues. Early detection of OA in the Destabilizing Medial Meniscus (DMM) mouse OA model using MB to detect induction of MMP-13 transcript, a major matrix proteinase that degrades interstitial collagen matrix during arthritis was shown. In vivo delivery of MMP13 MB using Nanopieces derived from rosette nanotubes were used. Since cartilage is a very negatively charged tissue (containing a huge amount of proteoglycan), the negatively charged Nanopieces intend to bind and accumulate onto and/or into the matrix and/or tissue resulting in much longer retention time to achieve more effective delivery. Different sizes of Nanopieces can be created for different delivery proposes to get into the matrix. For example, cartilage tissue matrix has about 60 nm mesh size of the collagen II fibrillar network and about 20 nm spacing between the side chains of the proteoglycan network. Nanopieces with small sizes (at least one dimension smaller than 60 nm and/or 20 nm) showed excellent efficiency and function in intra-cartilage matrix delivery of siRNA. Adjusting the ratio between RNTs and cargo reagents to yield an overall positive charged surface enabled Nanopieces to adhere with negatively charged matrix and/or tissue components resulting longer retention time.

Intra-joint delivery was thereby achieved with these processed Nanopieces. Delivery of Molecular probes with Nanopiece detected a specific gene expression (or protein activity) along with the co-delivery of a negative control for non-specific signal and an internal positive control to accurately diagnose a target gene expression in a real-time, in-situ and non-invasive manner. Matrix metalloproteinases (MMP) are the major enzymes that degrade the components of the extracellular matrix during arthritis progression. MMP-13, which is usually produced by cartilage and bone, degrade interstitial collagens (types I, II and III) in both OA and RA. Expression of MMP-13 is low in normal cells, whereas in pathologic condition excess MMP-13 production is associated with inflammation. mRNA level of MMP-13 are indicative for arthritis development and MMP-13 is as a good target in early diagnosis of arthritis. However, articular cartilage tissues need to be collected to show the up-regulation of MMP-13 mRNA levels. The combination of molecular beacon and Nanopieces technology detected of OA in vivo in a specific and sensitive manner without harming any joint tissues.

In another example, therapeutic agents complexed with nanotubes can knock down one or multiple disease gene expression (such as via siRNA delivery) and/or up-regulate one or multiple beneficial gene and/or protein (such as via DNA, mRNA or protein delivery) and deliver a variety of cargo types and can deliver multiple cargo reagents at the same time.

Accordingly, the rosette nanotubes of the present disclosure have hollow channels that can be used for drug encapsulation. Rosette nanotubes are able to incorporate water-insoluble drugs into their tubular structures by hydrophobic interactions with the core whereas their hydrophilic outer surface can shield such hydrophobic drugs in a physiological environment for subsequent prolonged release (even into the cell). Rosette nanotubes can also be chemically functionalized with peptides such as Arg-Gly-Asp-Ser-Lys (SEQ ID NO: 235), Lys-Arg-Ser-Arg-Lys (SEQ ID NO: 236), and Gly-Arg-Gly-Asp-Tyr-Lys (SEQ ID NO: 237) to deliver growth factors for healthy tissue regeneration, such as healthy bone in osteosarcoma patients, after the delivery of drugs to kill cancer cells.

The rosette nanotubes may also be used in tissue engineering, where living cells are utilized as engineering materials. Applications for tissue engineering are used to repair or replace portions of whole tissues such as bone, cartilage, blood vessels, muscle, etc. Tissues are fabricated in the laboratory from combinations of engineered extracellular matrices (“scaffolds”), cells, and biologically active molecules destined for transplantation. For example, nasal chondrocytes can expand in culture to engineer a cartilage graft. The rosette nanotubes of the current disclosure can be used as scaffolds in tissue engineering methods, e.g. using nasal chondrocytes, as well as a transfer vehicle to deliver therapeutic agents to specific tissues, e.g. cartilage, when using tissue engineering techniques known to a skilled person in the art.

### Genes and Proteins Used as Agents/Delivery Cargo

The following Genes and Proteins can be used as agents to complex with Nanotubes and Nanopieces:

The following Genes and Proteins can be used as target gene of siRNA which complex with Nanotubes and Nanopieces:

The mRNA transcript sequence encoding human ADAMTS-5, provided by Genbank Accession No. NM_007038.3, is incorporated herein by reference, and is shown below (SEQ ID NO: 1).

| | |
|---|---|
| 1 | ataaattcat tgttccacct cctcgcatct tcacagcgct cgcgctgctc tcggcgctcg |
| 61 | cagctgccga ctggggatga cggcgggcag gaggagaccg cagccgaagg gacacagaca |
| 121 | cgccgcttca ccagctcgcc tcaggctgcc cccctgcatt tttgttttaa tttttacggc |
| 181 | tttttcccct ctctttcttc ccttcctcct ggtcccagca gagccaagga aacccacaaa |
| 241 | ataagaaagg aagtgggccc cggagcttgg aacctccaca gccggcttgt ccagcgcagc |
| 301 | gcgggggcgg gaggctgcgc gcaccagttg ccagcccggt gcgcggtacc tttccttact |
| 361 | tttcttgaaa cagcgatcgt gcctgcattt ggtggttttt tggtttttgt ttttttcctt |
| 421 | ttcccgtatt tgctgaatct ccactatccg actttttttt tttaatcttt tctttccccc |
| 481 | cccccccacc ccacctcttt ctggagcacg aatccaaaca ttttcccaag caacaaagaa |
| 541 | aagttcgcac gctggcaccg cagcccggac aggctggcgc tgctgccggg cccccctccc |
| 601 | tccgacactt gactcaatcc tgcaagcaag tgtgtgtgtg tccccatccc ccgccccgtt |
| 661 | aacttcatag caaataacaa atacccataa agtcccagtc gcgcagcccc tccccgcggg |
| 721 | cagcgcacta tgctgctcgg gtgggcgtcc ctgctgctgt gcgcgttccg cctgcccctg |
| 781 | gccgcggtcg gccccgccgc gacacctgcc caggataaag ccgggcagcc tccgactgct |
| 841 | gcagcagccg cccagccccg ccggcggcag ggggaggagg tgcaggagcg agccgagcct |
| 901 | cccggccacc cgcaccccct ggcgcagcgg cgcaggagca aggggctggt gcagaacatc |
| 961 | gaccaactct actccggcgg cggcaaggtg ggctacctcg tctacgcggg cggccggagg |
| 1021 | ttcctcttgg acctggagcg agatggttcg gtgggcattg ctggcttcgt gcccgcagga |
| 1081 | ggcgggacga gtgcgccctg gcgccaccgg agccactgct tctatcgggg cacagtggac |
| 1141 | ggtagtcccc gctctctggc tgtctttgac ctctgtgggg gtctcgacgg cttcttcgcg |
| 1201 | gtcaagcacg cgcgctacac cctaaagcca ctgctgcgcg gaccctgggc ggaggaagaa |
| 1261 | aaggggcgcg tgtacgggga tgggtccgca cggatcctgc acgtctacac ccgcgagggc |
| 1321 | ttcagcttcg aggccctgcc gccgcgcgcc agctgcgaaa cccccgcgtc cacaccggag |
| 1381 | gcccacgagc atgctccggc gcacagcaac ccgagcggac gcgcagcact ggcctcgcag |
| 1441 | ctcttggacc agtccgctct ctcgcccgct gggggctcag gaccgcagac gtggtggcgg |
| 1501 | cggcggcgcc gctccatctc ccgggcccgc caggtggagc tgcttctggt ggctgacgcg |
| 1561 | tccatggcgc ggttgtatgg ccggggcctg cagcattacc tgctgaccct ggcctccatc |
| 1621 | gccaataggc tgtacagcca tgctagcatc gagaaccaca tccgcctggc cgtggtgaag |
| 1681 | gtggtggtgc taggcgacaa ggacaagagc ctggaagtga gcaagaacgc tgccaccaca |
| 1741 | ctcaagaact tttgcaagtg gcagcaccaa cacaaccagc tgggagatga ccatgaggag |
| 1801 | cactacgatg cagctatcct gtttactcgg gaggatttat gtgggcatca ttcatgtgac |
| 1861 | accctgggaa tggcagacgt tgggaccata tgttctccag agcgcagctg tgctgtgatt |
| 1921 | gaagacgatg gcctccacgc agccttcact gtggctcacg aaatcggaca tttacttggc |
| 1981 | ctctcccatg acgattccaa attctgtgaa gagacctttg gttccacaga agataagcgc |
| 2041 | ttaatgtctt ccatccttac cagcattgat gcatctaagc cctggtccaa atgcacttca |
| 2101 | gccaccatca cagaattcct ggatgatggc catggtaact gtttgctgga cctaccacga |
| 2161 | aagcagatcc tgggccccga agaactccca ggacagacct acgatgccac ccagcagtgc |
| 2221 | aacctgacat tcgggcctga gtactccgtg tgtcccggca tggatgtctg tgctcgcctg |
| 2281 | tggtgtgctg tggtacgcca gggccagatg gtctgtctga ccaagaagct gcctgcggtg |
| 2341 | gaagggacgc cttgtggaaa ggggagaatc tgcctgcagg gcaaatgtgt ggacaaaacc |
| 2401 | aagaaaaaat attattcaac gtcaagccat ggcaactggg gatcttgggg atcctggggc |
| 2461 | cagtgttctc gctcatgtgg aggaggagtg cagtttgcct atcgtcactg taataaccct |
| 2521 | gctcccagaa acaacggacg ctactgcaca gggaagaggg ccatctaccg ctcctgcagt |
| 2581 | ctcatgccct gcccacccaa tggtaaatca tttcgtcatg aacagtgtga ggccaaaaat |
| 2641 | ggctatcagt ctgatgcaaa aggagtcaaa acttttgtgg aatgggttcc caaatatgca |
| 2701 | ggtgtcctgc cagcggatgt gtgcaagctg acctgcagag ccaagggcac tggctactat |
| 2761 | gtggtatttt ctccaaaggt gaccgatggc actgaatgta ggctgtacag taattccgtc |
| 2821 | tgcgtccggg ggaagtgtgt gagaactggc tgtgacggca tcattggctc aaagctgcag |
| 2881 | tatgacaagt gcggagtatg tggaggagac aactccagct gtacaaagat tgttggaacc |
| 2941 | tttaataaga aaagtaaggg ttacactgac gtggtgagga ttcctgaagg ggcaacccac |
| 3001 | ataaaagttc gacagttcaa agccaaagac cagactagat tcactgccta tttagccctg |
| 3061 | aaaaagaaaa acggtgagta ccttatcaat ggaaagtaca tgatctccac ttcagagact |
| 3121 | atcattgaca tcaatggaac agtcatgaac tatagcggtt ggagccacag ggatgacttc |
| 3181 | ctgcatggca tgggctactc tgccacgaag gaaattctaa tagtgcagat tcttgcaaca |
| 3241 | gaccccacta aaccattaga tgtccgttat agcttttttg ttcccaagaa gtccactcca |
| 3301 | aaagtaaact ctgtcactag tcatggcagc aataaagtgg gatcacacac ttcgcagccg |
| 3361 | cagtgggtca cgggcccatg gctcgcctgc tctaggacct gtgacacagg ttggcacacc |
| 3421 | agaacggtgc agtgccagga tggaaaccgg aagttagcaa aaggatgtcc tctctcccaa |
| 3481 | aggccttctg cgtttaagca atgcttgttg aagaaatgtt agcctgtggt tatgatctta |
| 3541 | tgcacaaaga taactggagg attcagcact gatgcagtcg tggtgaacag gaggtctacc |
| 3601 | taacgcacag aaagtcatgc ttcagtgaca ttgtcaacag gagtccaatt atgggcagaa |
| 3661 | tctgctctct gtgaccaaaa gaggatgtgc actgcttcac gtgacagtgg tgaccttgca |
| 3721 | atatagaaaa acttgggagt tattgaacat cccctgggct tacaagaaac actgatgaat |
| 3781 | gtaaaatcag gggacatttg aagatggcag aactgtctcc cccttgtcac ctacctctga |
| 3841 | tagaatgtct ttaatggtat cataatcatt ttcacccata atacacagta gcttcttctt |
| 3901 | actgtttgta aatacattct cccttggtat gtcactttat atcccctggt tctattaaaa |
| 3961 | tatccatata tatttctata aaaaaagtgt ttgaccaaag taggtctgca gctatttcaa |
| 4021 | cttccttccg tttccagaaa gagctgtgga tattttactg gaaattaaga acttgctgct |
| 4081 | gttttaataa gatgtagtat attttctgac tacaggagat aaaatttcag tcaaaaaacc |
| 4141 | attttgacag caagtatctt ctgagaaatt ttgaaaagta aatagatctc agtgtatcta |
| 4201 | gtcacttaaa tacatacacg ggttcattta cttaaacctt tgactgcctg tatttttttc |
| 4261 | aggtagctag ccaaattaat gcataatttc agatgtagaa gtagggtttg cgtgtgtgtg |
| 4321 | tgtgatcata ctcaagagtc taaaaactag tttccttgtg ttggaaattt aaaaggaaaa |
| 4381 | aaatcgtatt tcactgtgtt ttcaatttat attttcacaa ctactttctc tctccagagc |
| 4441 | tttcatctga tatctcacaa tgtatgatat acgtacaaaa cacacagcaa gttttctatc |
| 4501 | atgtccaaca cattcaacac tggtatacct cctaccagca agcctttaaa atgcatttgt |
| 4561 | gtttgcttat ttgttttgtt caagggttca gtaagaccta caatgttttg tatttcttga |
| 4621 | cttattttat tagaaacatt aaagatcact tggtagttag ccacattgag aagtggttat |
| 4681 | cattgttaat gtggttaatg ccaaaaagtg gttaatatta ataagactgt ttccacacca |
| 4741 | taggcaataa tttcttaatt taaaaaatct aagtatattc ctattgtact aaatattttt |
| 4801 | cccaactgga aagcacttga ttgtacccgt aagtgtttga gtgatgacat gtgatgattt |
| 4861 | tcagaaagtt gttgtttttg tttccatagc ctgtttaagt aggttgtaag tttgaatagt |
| 4921 | tagacatgga aattatttta taagcacaca cctaaagata tctttttaga tgataaaatg |
| 4981 | tacacccccc catcaccaac ctcacaactt agaaaatcta agttgtttga tttctttggg |
| 5041 | atttcttttg ttgtgaaaca ctgcaaagcc aatttttctt tataaaaatt catagtaatc |
| 5101 | ctgccaaatg tgcctattgt taaagatttg catgtgaaga tcttagggaa ccactgtttg |
| 5161 | agttctacaa gctcatgaga gtttattttt attataagat gtttttaata taaaagaatt |
| 5221 | atgtaactga tcactatatt acatcatttc agtgggccag gaaaatagat gtcttgctgt |
| 5281 | tttcagtatt ttcttaagaa attgctttta aaacaaataa ttgttttaca aaaccaataa |
| 5341 | ttatcctttg aattttcata gactgacttt gcttttgacg tagaaatttt ttttctcaat |
| 5401 | aaattatcac tttgagaaat gaggcctgta caaggctgat aacctatatg tgatggagat |
| 5461 | cacccaatgc caagggcaga aagcaaacct agttaaatag gtgagaaaaa aaataataat |
| 5521 | cccagtgcca tttgtctgtg caaagagaat taggagagag gttaatgtta cttttttcca |
| 5581 | ttttggaaat aattttaatc aagtaactca aatgtgacaa aatttatttt tattttttgt |
| 5641 | ggttatattc ccaacaacat taaaaaatac tcgaggcata aatgtagttg tctcctactc |
| 5701 | tgcttctctt actatactca tacattttta atatggttta tcaatgattc atgtttccct |
| 5761 | caaatagtga tggtttacac ctgtcatgga aacaatccta gagagctcag agcaattaaa |
| 5821 | ccactattcc atgcttttaa gtagttttct ccaccttttt cttatgagtc tcactagatt |
| 5881 | gactgaggaa tgtatgtcta aattcctgga gaagatgata tggattggaa actgaaattc |
| 5941 | agagaaatgg agtgttcaat agataccacg aattgtgaac aaagggaaaa ttctatacaa |
| 6001 | ctcaatctaa gtcagtccac tttgacttcg tactgtcttt cacctttcca ttgttgcatc |
| 6061 | ttgaattttt taaaatgtct agaattcagg atgctagggg ctacttcttt aaaaaaaaaa |
| 6121 | aaaaaaaaga attcgtctga aaatgctcag gtttgtaaga atctaatctc acttacataa |
| 6181 | ctaagcactc cataataagt tttattaagt acaaagggag ccagaaaaaa tgacatttat |
| 6241 | ttcttctaga tcagaaaaat ttaaattaag ccctgccttg ctgtttagaa atatgtgggc |
| 6301 | attgttataa tttattcaat aaatttatgt tcctttgcct tcctgtggaa acagttttat |
| 6361 | cccactaaac taggaattag gggataaatc acaaacaaaa aaaaagttgc agcactgaaa |
| 6421 | aaaagtaatt tattgttttt gcaactggta tgtgaatttg tgtgataaaa ttatttattc |
| 6481 | ttatttaaca aaaatatgtt caaatttttc tatatttaaa atgttttgct gttgtcctac |
| 6541 | tttttaattt atgcttcatg tttgtgtata aagtacactt ttacactttg tgagtttaca |
| 6601 | taatatacag cactggttgc ttttgtattt ttttacagaa agctttctgt gtgaagcagg |
| 6661 | tgtatatgta tatattcctc atgtattctt attctgatac tatcattttt ctttccaagg |
| 6721 | aaattttaat ctgtcatgac caatagtgtt cattacttgt gcctatgata ataggttttt |
| 6781 | tacatcacat taacactatt ttttccaagt cacaaataag aaaaacactt attcaatgaa |
| 6841 | acaaggtgca agttttaaat ttgggtacac aaatagccta gaagcttcct acagacgcta |
| 6901 | agacacagcc aataatcaga tcctttcact tcatcgagaa acttggacaa gtcgatattg |
| 6961 | atgtattaga tgaaagttgt ctacacacaa cttctgaggg atacaaacga taataaaacc |
| 7021 | aaatgttgtc tgtttctcct ttagaaacac ctcctaaaat taatatcatt tagtctctag |
| 7081 | tgtctgtagg attctacaga tgagcacaaa tagattgggt ttgtataaca aatgctaata |
| 7141 | gtcataactg tttctacaaa tatggggtgt ccattaagag aatgtgatgt tttcctactg |
| 7201 | ctgttgaatc ccatggggtg attataggac ttgaaatagg cagagtcacc tctgatgaca |
| 7261 | tcagcttgcc tctgtgattt cacagtctga tcctggcaac aagacaaagc acccttggac |
| 7321 | acacagccaa tctctggttg tgatatttcc ccattgattc cttccttgtt aacaaggtca |
| 7381 | ttttaatggt tcaggtgagg acagcagcca gattcaaagt ccagaatttg tgctgttaca |
| 7441 | tagagttcac actgtcaaat aacattgaat ttaataatga tcaaattttt ctagtagtct |
| 7501 | ttggcagagt gtataatctc attggcatga ttggtgaata ttactaatct ctttataatg |
| 7561 | aaagatgctt tacaaatacc ttatatttgc taacatttca aaactactaa ataaatgaaa |
| 7621 | tagccatgtg tacagaaatg gtcatttaaa gctttaatag aaccaaattc aagacaatgt |
| 7681 | atcatttaga cacacagaaa aggaacttgt atgttttccc tattattttt ctcatttgcc |
| 7741 | aacaatctat agttttaggt tatcaaacag atagatcaac ttaactggct agtacattga |
| 7801 | aaaatcttcc taagaatcct ttgttagcat aatctataga gataatttct caaattatat |
| 7861 | catcatgatg catataaact ctataatgta taattgtgtt tcatttattt aatgtatgag |
| 7921 | aacatattga aatacaaaac catgcattag ccaaaaaatt ggaatacagg tagtgttcag |
| 7981 | atcagcaaaa cattcagtct ggtaaatgcc tgcctggggc tatgatatca ttctcaatgc |
| 8041 | aggttttatg gaaaaactaa aagaatatgt tgttagatga tgttggtttt gaaaaaaaaa |
| 8101 | agacattaac atacacatta gttagcccag ttaattgcat tctactaata tagttgcaca |
| 8161 | ttagcaataa ttttgctgtc tctggtcttt attttgtggc ttcaactaac tggaccatgt |
| 8221 | ggactgtaaa ggtcaaatgg aaaaaacgag cagtggcccc tcatcctgta aggtactgct |
| 8281 | acatcagagt gacctaaaag tctaacactg tgaggaaaac tgtgatttgt aggaaaaaaa |
| 8341 | aaaaaaacaa ataaaaaaca gggcatgctt tttaattttt ttccactttc ctttggcaca |
| 8401 | cccaatgaac aattctaatt tttattgagg tgctaacatc tttcgtgacc gactgtcaaa |
| 8461 | tgtggtattt ttgagttact atttttctac atgattttac agtttgcaag aaagacctct |
| 8521 | aagctttgtg tcacggtagg gcacaacttg atactcaaaa tttgaaaaat aagcacatcc |
| 8581 | aatgattgtt ttgaccaaca gtggtcagtg acgtaaactg catgtgcatc tgaggacatt |
| 8641 | taaggggtca ttaaaatttg aggagcatca ggccggagta gcagactttt agatgagtca |
| 8701 | tatttcagca ttcactaagt cctcagcatt ccattcaaac tgtcgtgtat atttggcctg |
| 8761 | attttttttc aagctttgca ataatttatg ttattggtaa acacttggtg actatatctc |
| 8821 | agccttttct ttaacaactc acaatatatt agaaacacgt ctacctatac tgagagtata |
| 8881 | tttacaatag aagaacatac tgtatgtgac tttgtaaagc tagacttttg attaagaaat |
| 8941 | atataatctc tggatgctat ttttgcatta tacactcagg cacaacgtaa accttgatgg |
| 9001 | ctcatcttgc tacaattacg agttgaaaaa cactacttac gtatttgtat gacctattag |
| 9061 | tcagaggaaa tcatacatat gctttgtaaa tagactttgc agataactaa atagactgaa |
| 9121 | gaaatatgtt gcatttgata gaagcaattg cataaatatt tggtttctat attagagtct |
| 9181 | gtgagtaaag tcaagtaata aacctaagta ggtataacag atttttaaac cttgaaactt |
| 9241 | gctttgatgg tagagaaaat cattgaagat ttacatactg tatataagat gtaaaatgta |
| 9301 | cgctgcttat taccctcaat tttccagaag caatggtata taatgcagtt gaaaaaccaa |
| 9361 | aaatcttgga aaactaagac gggtcttgtt taaaatgtct ctcagctttg gcaaccttca |
| 9421 | aatcttaatc aactatttaa agcattactg tgtcttgtag cctgcattcc acaacagctc |
| 9481 | tgttattcag gtaaaagact tgaactgagc cgtttgggac ctatactgta atattttcat |
| 9541 | tgaggaacaa tatcctattt tgtaaagcat ttccctatgt gtgactttaa actgtaaaat |
| 9601 | taaacactgc ttttgtgggt tcagtgggca taataaatat aaattgtaaa ctaggttaaa |
| 9661 | gta |

The amino acid sequence of human ADAMTS-5 (preproprotein), provided by Genbank Accession No. NP_008969.2, is incorporated herein by reference, and is shown below (SEQ ID NO: 2).

| | |
|---|---|
| 1 | mllgwaslll cafrlplaav gpaatpaqdk agqpptaaaa |
| | aqprrrqgee vqeraeppgh |
| 61 | phplaqrrrs kglvqnidql ysgggkvgyl vyaggrrfll |
| | dlerdgsvgi agfvpagggt |
| 121 | sapwrhrshc fyrgtvdgsp rslavfdlcg gldgffavkh |
| | arytlkpllr gpwaeeekgr |
| 181 | vygdgsaril hvytregfsf ealpprasce tpastpeahe |
| | hapahsnpsg raalasqlld |
| 241 | qsalspaggs gpqtwwrrrr rsisrarqve lllvadasma |
| | rlygrglqhy lltlasianr |
| 301 | lyshasienh irlavvkvvv lgdkdkslev sknaattlkn |
| | fckwqhqhnq lgddheehyd |
| 361 | aailftredl cghhscdtlg madvgticsp erscaviedd |
| | glhaaftvah eighllglsh |
| 421 | ddskfceetf gstedkrlms siltsidask pwskctsati |
| | teflddghgn clldlprkqi |
| 481 | lgpeelpgqt ydatqqcnlt fgpeysvcpg mdvcarlwca |
| | vvrqgqmvcl tkklpavegt |
| 541 | pcgkgriclq gkcvdktkkk yystsshgnw gswgswgqcs |
| | rscgggvqfa yrhcnnpapr |
| 601 | nngryctgkr aiyrscslmp cppngksfrh eqceakngyq |
| | sdakgvktfv ewvpkyagvl |
| 661 | padvckltcr akgtgyyvvf spkvtdgtec rlysnsvcvr |
| | gkcvrtgcdg iigsklqydk |
| 721 | cgvcggdnss ctkivgtfnk kskgytdvvr ipegathikv |
| | rqfkakdqtr ftaylalkkk |
| 781 | ngeylingky mistsetiid ingtvmnysg wshrddflhg |
| | mgysatkeil ivqilatdpt |
| 841 | kpldvrysff vpkkstpkvn svtshgsnkv gshtsqpqwv |
| | tgpwlacsrt cdtgwhtrtv |
| 901 | qcqdgnrkla kgcplsqrps afkqcllkkc |
| | (Signal peptide AA 1-6; proprotein AA 17-930; |
| | mature peptide AA 262-930. |

The siRNA used to target human ADAMTS-5 mRNA include following sequences (SEQ ID NO: 3-6):

SEQ NO: 3:
5′-GCUCAAAGCUGCAGUAUGA-3′
SEQ NO: 4:
5′-GAAGUCCACUCCAAAAGUA-3′
SEQ NO: 5:
5′-GCACUACGAUGCAGCUAUC-3′
SEQ NO: 6:
5′-CGAAGGAAAUUCUAAUAGU-3′

The molecular beacon used to target human ADAMTS-5 mRNA includes the following sequences (SEQ ID NO: 7-9):

SEQ NO 7:
5′-CCGGTC TAACATTTCTTCAACAAGCA GACCGG-3′
SEQ NO 8:
5′-CCGGTC TTATACACAAACATGAAGCA GACCGG-3′
SEQ NO 9:
5′-CCGGTC TACATCTTATTAAAACAGCA GACCGG-3′

The mRNA transcript sequence encoding human ADAMTS-4, provided by Genbank Accession No. NM_005099.4, is incorporated herein by reference, and is shown below (SEQ ID NO: 10).

| | |
|---|---|
| 1 | ggggagaacc cacagggaga cccacagaca catatgcacg agagagacag aggaggaaag |
| 61 | agacagagac aaaggcacag cggaagaagg cagagacagg gcaggcacag aagcggccca |
| 121 | gacagagtcc tacagaggga gaggccagag aagctgcaga agacacaggc agggagagac |
| 181 | aaagatccag gaaaggaggg ctcaggagga gagtttggag aagccagacc cctgggcacc |
| 241 | tctcccaagc ccaaggacta agttttctcc atttccttta acggtcctca gcccttctga |
| 301 | aaactttgcc tctgaccttg gcaggagtcc aagcccccag gctacagaga ggagctttcc |
| 361 | aaagctaggg tgtggaggac ttggtgccct agacggcctc agtccctccc agctgcagta |
| 421 | ccagtgccat gtcccagaca ggctcgcatc ccgggagggg cttggcaggg cgctggctgt |
| 481 | ggggagccca accctgcctc ctgctcccca ttgtgccgct ctcctggctg gtgtggctgc |
| 541 | ttctgctact gctggcctct ctcctgccct cagcccggct ggccagcccc ctcccccggg |
| 601 | aggaggagat cgtgtttcca gagaagctca acggcagcgt cctgcctggc tcgggcgccc |
| 661 | ctgccaggct gttgtgccgc ttgcaggcct ttggggagac gctgctacta gagctggagc |
| 721 | aggactccgg tgtgcaggtc gaggggctga cagtgcagta cctgggccag gcgcctgagc |
| 781 | tgctgggtgg agcagagcct ggcacctacc tgactggcac catcaatgga gatccggagt |
| 841 | cggtggcatc tctgcactgg gatgggggag ccctgttagg cgtgttacaa tatcgggggg |
| 901 | ctgaactcca cctccagccc ctggagggag gcacccctaa ctctgctggg ggacctgggg |
| 961 | ctcacatcct acgccggaag agtcctgcca gcggtcaagg tcccatgtgc aacgtcaagg |
| 1021 | ctcctcttgg aagccccagc cccagacccc gaagagccaa gcgctttgct tcactgagta |
| 1081 | gatttgtgga gacactggtg gtggcagatg acaagatggc cgcattccac ggtgcggggc |
| 1141 | taaagcgcta cctgctaaca gtgatggcag cagcagccaa ggccttcaag cacccaagca |
| 1201 | tccgcaatcc tgtcagcttg gtggtgactc ggctagtgat cctggggtca ggcgaggagg |
| 1261 | ggccccaagt ggggcccagt gctgcccaga ccctgcgcag cttctgtgcc tggcagcggg |
| 1321 | gcctcaacac ccctgaggac tcggaccctg accactttga cacagccatt ctgtttaccc |
| 1381 | gtcaggacct gtgtggagtc tccacttgcg acacgctggg tatggctgat gtgggcaccg |
| 1441 | tctgtgaccc ggctcggagc tgtgccattg tggaggatga tgggctccag tcagccttca |
| 1501 | ctgctgctca tgaactgggt catgtcttca acatgctcca tgacaactcc aagccatgca |
| 1561 | tcagtttgaa tgggcctttg agcacctctc gccatgtcat ggcccctgtg atggctcatg |
| 1621 | tggatcctga ggagccctgg tccccctgca gtgcccgctt catcactgac ttcctggaca |
| 1681 | atggctatgg gcactgtctc ttagacaaac cagaggctcc attgcatctg cctgtgactt |
| 1741 | tccctggcaa ggactatgat gctgaccgcc agtgccagct gaccttcggg cccgactcac |
| 1801 | gccattgtcc acagctgccg ccgccctgtg ctgccctctg gtgctctggc cacctcaatg |
| 1861 | gccatgccat gtgccagacc aaacactcgc cctgggccga tggcacaccc tgcgggcccg |
| 1921 | cacaggcctg catgggtggt cgctgcctcc acatggacca gctccaggac ttcaatattc |
| 1981 | cacaggctgg tggctggggt ccttggggac catggggtga ctgctctcgg acctgtgggg |
| 2041 | gtggtgtcca gttctcctcc cgagactgca cgaggcctgt cccccggaat ggtggcaagt |
| 2101 | actgtgaggg ccgccgtacc cgcttccgct cctgcaacac tgaggactgc ccaactggct |
| 2161 | cagccctgac cttccgcgag gagcagtgtg ctgcctacaa ccaccgcacc gacctcttca |
| 2221 | agagcttccc agggcccatg gactgggttc ctcgctacac aggcgtggcc ccccaggacc |
| 2281 | agtgcaaact cacctgccag gcccaggcac tgggctacta ctatgtgctg gagccacggg |
| 2341 | tggtagatgg gaccccctgt tccccggaca gctcctcggt ctgtgtccag ggccgatgca |
| 2401 | tccatgctgg ctgtgatcgc atcattggct ccaagaagaa gtttgacaag tgcatggtgt |
| 2461 | gcggagggga cggttctggt tgcagcaagc agtcaggctc cttcaggaaa ttcaggtacg |
| 2521 | gatacaacaa tgtggtcact atccccgcgg gggccaccca cattcttgtc cggcagcagg |
| 2581 | gaaaccctgg ccaccggagc atctacttgg ccctgaagct gccagatggc tcctatgccc |
| 2641 | tcaatggtga atacacgctg atgccctccc ccacagatgt ggtactgcct ggggcagtca |
| 2701 | gcttgcgcta cagcggggcc actgcagcct cagagacact gtcaggccat gggccactgg |
| 2761 | cccagccttt gacactgcaa gtcctagtgg ctggcaaccc ccaggacaca cgcctccgat |
| 2821 | acagcttctt cgtgccccgg ccgacccctt caacgccacg ccccactccc caggactggc |
| 2881 | tgcaccgaag agcacagatt ctggagatcc ttcggcggcg cccctgggcg ggcaggaaat |
| 2941 | aacctcacta tcccggctgc cctttctggg caccggggcc tcggacttag ctgggagaaa |
| 3001 | gagagagctt ctgttgctgc ctcatgctaa gactcagtgg ggaggggctg tgggcgtgag |
| 3061 | acctgcccct cctctctgcc ctaatgcgca ggctggccct gccctggttt cctgccctgg |
| 3121 | gaggcagtga tgggttagtg gatggaaggg gctgacagac agccctccat ctaaactgcc |
| 3181 | ccctctgccc tgcgggtcac aggagggagg gggaaggcag ggagggcctg ggccccagtt |
| 3241 | gtatttattt agtatttatt cacttttatt tagcaccagg gaaggggaca aggactaggg |
| 3301 | tcctggggaa cctgacccct gacccctcat agccctcacc ctggggctag gaaatccagg |
| 3361 | gtggtggtga taggtataag tggtgtgtgt atgcgtgtgt gtgtgtgtga aaatgtgtgt |
| 3421 | gtgcttatgt atgaggtaca acctgttctg ctttcctctt cctgaatttt attttttggg |
| 3481 | aaaagaaaag tcaagggtag ggtgggcctt cagggagtga gggattatct tttttttttt |
| 3541 | ttctttcttt ctttcttttt tttttttgag acagaatctc gctctgtcgc ccaggctgga |
| 3601 | gtgcaatggc acaatctcgg ctcactgcat cctccgcctc ccgggttcaa gtgattctca |
| 3661 | tgcctcagcc tcctgagtag ctgggattac aggctcctgc caccacgccc ggctaatttt |
| 3721 | tgttttgttt tgtttggaga cagagtctcg ctattgtcac cagggctgga atgatttcag |
| 3781 | ctcactgcaa ccttcgccac ctgggttcca gcaattctcc tgcctcagcc tcccgagtag |
| 3841 | ctgagattat aggcacctac caccacgccc ggctaatttt tgtattttta gtagagacgg |
| 3901 | ggtttcacca tgttggccag gctggtctcg aactcctgac cttaggtgat ccactcgcct |
| 3961 | tcatctccca aagtgctggg attacaggcg tgagccaccg tgcctggcca cgcccaacta |
| 4021 | atttttgtat ttttagtaga gacagggttt caccatgttg gccaggctgc tcttgaactc |
| 4081 | ctgacctcag gtaatcgacc tgcctcggcc tcccaaagtg ctgggattac aggtgtgagc |
| 4141 | caccacgccc ggtacatatt ttttaaattg aattctacta tttatgtgat ccttttggag |
| 4201 | tcagacagat gtggttgcat cctaactcca tgtctctgag cattagattt ctcatttgcc |
| 4261 | aataataata cctcccttag aagtttgttg tgaggattaa ataatgtaaa taaagaacta |
| 4321 | gcataacact caaaaaaaaa aaaaaaaaaa aaaaaaaaaa aaaaaaaaaa aaaaaaaaaa |
| 4381 | aaaaaaaaaa aaaaaaaaaa aaaaaaaaaa |

The amino acid sequence of human ADAMTS-4 (preproprotein), provided by Genbank Accession No. NP_005090.3, is incorporated herein by reference, and is shown below (SEQ ID NO: 11).

| | |
|---|---|
| 1 | msqtgshpgr glagrwlwga qpclllpivp lswlvwllll |
| | llasllpsar lasplpreee |
| 61 | ivfpeklngs vlpgsgapar llcrlqafge tllleleqds |
| | gvqvegltvq ylgqapellg |
| 121 | gaepgtyltg tingdpesva slhwdggall gvlqyrgael |
| | hlqpleggtp nsaggpgahi |
| 181 | lrrkspasgq gpmcnvkapl gspsprprra krfaslsrfv |
| | etlvvaddkm aafhgaglkr |
| 241 | ylltvmaaaa kafkhpsirn pvslvvtrlv ilgsgeegpq |
| | vgpsaaqtlr sfcawqrgln |
| 301 | tpedsdpdhf dtailftrqd lcgstcdtl gmadvgtvcd |
| | parscaived dglqsaftaa |
| 361 | helghvfnml hdnskpcisl ngplstsrhv mapvmahvdp |
| | eepwspcsar fitdfldngy |
| 421 | ghclldkpea plhlpvtfpg kdydadrqcq ltfgpdsrhc |
| | pqlpppcaal wcsghlngha |
| 481 | mcqtkhspwa dgtpcgpaqa cmggrclhmd qlqdfnipqa |
| | ggwgpwgpwg dcsrtcgggv |
| 541 | qfssrdctrp vprnggkyce grrtrfrscn tedcptgsal |
| | tfreeqcaay nhrtdlfksf |
| 601 | pgpmdwvpry tgvapqdqck ltcqaqalgy yyvleprvvd |
| | gtpcspdsss vcvqgrciha |
| 661 | gcdriigskk kfdkcmvcgg dgsgcskqsg sfrkfrygyn |
| | nvvtipagat hilvrqqgnp |
| 721 | ghrsiylalk lpdgsyalng eytlmpsptd vvlpgayslr |
| | ysgataaset lsghgplaqp |
| 781 | ltlqvlvagn pqdtrlrysf fvprptpstp rptpqdwlhr |
| | raqileilrr rpwagrk |

The siRNA used to target human ADAMTS-4 mRNA includes the following sequences (SEQ ID NO: 12-15):

SEQ NO: 12:
5′-CCGCAAUCCUGUCAGCUUG-3′
SEQ NO: 13:
5′-GCGCUUUGCUUCACUGAGU-3′
SEQ NO: 14:
5′-GGACACACGCCUCCGAUAC-3′
SEQ NO: 15:
5′-GCACCGAAGAGCACAGAUU-3′

The molecular beacon used to target human ADAMTS-4 mRNA includes the following sequences (SEQ ID NO: 16-18):

SEQ NO: 16:
5′-CCGGTC TTTTCACACACACACACACG GACCGG-3′
SEQ NO: 17:
5′-CCGGTC TAAAAATACAAAAATTAGCC GACCGG-3′
SEQ NO: 18:
5′-CCGGTC TTGTCTCTGTCTCTTTCCTC GACCGG-3′

The mRNA transcript sequence encoding human MMP-13, provided by Genbank Accession No. NM_002427.3, is incorporated herein by reference, and is shown below (SEQ ID NO: 19).

| | |
|---|---|
| 1 | acaacagtcc ccaggcatca ccattcaaga tgcatccagg |
| | ggtcctggct gccttcctct |
| 61 | tcttgagctg gactcattgt cgggccctgc cccttcccag |
| | tggtggtgat gaagatgatt |
| 121 | tgtctgagga agacctccag tttgcagagc gctacctgag |
| | atcatactac catcctacaa |
| 181 | atctcgcggg aatcctgaag gagaatgcag caagctccat |
| | gactgagagg ctccgagaaa |
| 241 | tgcagtcttt cttcggctta gaggtgactg gcaaacttga |
| | cgataacacc ttagatgtca |
| 301 | tgaaaaagcc aagatgcggg gttcctgatg tgggtgaata |
| | caatgttttc cctcgaactc |
| 361 | ttaaatggtc caaaatgaat ttaacctaca gaattgtgaa |
| | ttacacccct gatatgactc |
| 421 | attctgaagt cgaaaaggca ttcaaaaaag ccttcaaagt |
| | ttggtccgat gtaactcctc |
| 481 | tgaattttac cagacttcac gatggcattg ctgacatcat |
| | gatctctttt ggaattaagg |
| 541 | agcatggcga cttctaccca tttgatgggc cctctggcct |
| | gctggctcat gcttttcctc |
| 601 | ctgggccaaa ttatggagga gatgcccatt ttgatgatga |
| | tgaaacctgg acaagtagtt |
| 661 | ccaaaggcta caacttgttt cttgttgctg cgcatgagtt |
| | cggccactcc ttaggtcttg |
| 721 | accactccaa ggaccctgga gcactcatgt ttcctatcta |
| | cacctacacc ggcaaaagcc |
| 781 | actttatgct tcctgatgac gatgtacaag ggatccagtc |
| | tctctatggt ccaggagatg |
| 841 | aagaccccaa ccctaaacat ccaaaaacgc cagacaaatg |
| | tgacccttcc ttatcccttg |
| 901 | atgccattac cagtctccga ggagaaacaa tgatctttaa |
| | agacagattc ttctggcgcc |
| 961 | tgcatcctca gcaggttgat gcggagctgt ttttaacgaa |
| | atcattttgg ccagaacttc |
| 1021 | ccaaccgtat tgatgctgca tatgagcacc cttctcatga |
| | cctcatcttc atcttcagag |
| 1081 | gtagaaaatt ttgggctctt aatggttatg acattctgga |
| | aggttatccc aaaaaaatat |
| 1141 | ctgaactggg tcttccaaaa gaagttaaga agataagtgc |
| | agctgttcac tttgaggata |
| 1201 | caggcaagac tctcctgttc tcaggaaacc aggtctggag |
| | atatgatgat actaaccata |
| 1261 | ttatggataa agactatccg agactaatag aagaagactt |
| | cccaggaatt ggtgataaag |
| 1321 | tagatgctgt ctatgagaaa aatggttata tctatttttt |
| | caacggaccc atacagtttg |
| 1381 | aatacagcat ctggagtaac cgtattgttc gcgtcatgcc |
| | agcaaattcc attttgtggt |
| 1441 | gttaagtgtc tttttaaaaa ttgttattta aatcctgaag |
| | agcatttggg gtaatacttc |
| 1501 | cagaagtgcg gggtagggga agaagagcta tcaggagaaa |
| | gcttggttct gtgaacaagc |
| 1561 | ttcagtaagt tatctttgaa tatgtagtat ctatatgact |
| | atgcgtggct ggaaccacat |
| 1621 | tgaagaatgt tagagtaatg aaatggagga tctctaaaga |
| | gcatctgatt cttgttgctg |
| 1681 | tacaaaagca atggttgatg atacttccca caccacaaat |
| | gggacacatg gtctgtcaat |
| 1741 | gagagcataa tttaaaaata tatttataag gaaattttac |
| | aagggcataa agtaaataca |
| 1801 | tgcatataat gaataaatca ttcttactaa aaagtataaa |
| | atagtatgaa aatggaaatt |
| 1861 | tgggagagcc atacataaaa gaaataaacc aaaggaaaat |
| | gtctgtaata atagactgta |
| 1921 | acttccaaat aaataatttt cattttgcac tgaggatatt |
| | cagatgtatg tgcccttctt |
| 1981 | cacacagaca ctaacgaaat atcaaagtca ttaaagacag |
| | gagacaaaag agcagtggta |
| 2041 | agaatagtag atgtggcctt tgaattctgt ttaattttca |
| | cttttggcaa tgactcaaag |
| 2101 | tctgctctca tataagacaa atattccttt gcatattata |
| | aaggataaag aaggatgatg |
| 2161 | tctttttatt aaaatatttc aggttcttca gaagtcacac |
| | attacaaagt taaaattgtt |
| 2221 | atcaaaatag tctaaggcca tggcatccct ttttcataaa |
| | ttatttgatt atttaagact |
| 2281 | aaaagttgca ttttaaccct attttaccta gctaattatt |
| | taattgtcca gtttgtcttg |
| 2341 | gatatatagg ctattttcta aagacttgta tagcatgaaa |
| | taaaatatat cttataaagt |
| 2401 | ggaagtatgt atattaaaaa agagacatcc aaattttttt |
| | ttaaagcagt ctactagatt |
| 2461 | gtgatccctt gagatatgga aggatgcctt tttttctctg |
| | catttaaaaa aatcccccag |
| 2521 | cacttcccac agtgcctatt gatacttggg gagggtgctt |
| | ggcacttatt gaatatatga |
| 2581 | tcggccatca agggaagaac tattgtgctc agagacactg |
| | ttgataaaaa ctcaggcaaa |
| 2641 | gaaaatgaaa tgcatatttg caaagtgtat taggaagtgt |
| | ttatgttgtt tataataaaa |
| 2701 | atatattttc aacagacaaa aaaaaaaaaa aaaaa |

The amino acid sequence of human MMP-13 (collagenase 3 preproprotein), provided by Genbank Accession No. NP_002418.1, is incorporated herein by reference, and is shown below (SEQ ID NO: 20).

| | |
|---|---|
| 1 | mhpgvlaafl flswthcral plpsggdedd lseedlqfae |
| | rylrsyyhpt nlagilkena |
| 61 | assmterlre mqsffglevt gklddntldv mkkprcgvpd |
| | vgeynvfprt lkwskmnlty |
| 121 | rivnytpdmt hsevekafkk afkvwsdvtp lnftrlhdgi |
| | adimisfgik ehgdfypfdg |
| 181 | psgllahafp pgpnyggdah fdddetwtss skgynlflva |
| | ahefghslgl dhskdpgalm |
| 241 | fpiytytgks hfmlpdddvq giqslygpgd edpnpkhpkt |
| | pdkcdpslsl daitslrget |
| 301 | mifkdrffwr lhpqqvdael fltksfwpel pnridaayeh |
| | pshdlififr grkfwalngy |
| 361 | dilegypkki selglpkevk kisaavhfed tglallfsgn |
| | qvwryddtnh imdkdyprli |
| 421 | eedfpgigdk vdavyekngy iyffngpiqf eysiwsnriv |
| | rvmpansilw c |
| | (Signal protein AA 1-19; proprotein AA |
| | 20-471; mature peptide AA 104-471). |

The siRNA used to target human MMP-13 mRNA includes the following sequences (SEQ ID NO: 21-24):

SEQ NO: 21:
5′-UUUCACACACACACACACGC-3′
SEQ NO: 22:
5′-UUUUCACACACACACACACG-3′
SEQ NO: 23:
5′-UAAAAAUACAAAAAUUAGCC-3′
SEQ NO: 24:
5′-UUUGUCUCUGUCUCUUUCCU-3′

The molecular beacon used to target human MMP-13 mRNA includes the following sequences (SEQ ID NO: 25-27):

SEQ NO 25:
5′-CCGGTC TACACACACCACTTATACCT GACCGG-3′
SEQ NO 26:
5′-CCGGTC TATAATCTCAGCTACTCGGG GACCGG-3′
SEQ NO 27:
5′-CCGGTC AAACAAAACAAAAATTAGCC GACCGG-3′

The mRNA transcript sequence encoding human MMP-1 variant 2, provided by Genbank Accession No. NM_001145938.1, is incorporated herein by reference, and is shown below (SEQ ID NO: 28).

| | |
|---|---|
| 1 | agcatgagtc agacagcctc tggctttctg gaagggcaag |
| | gactctatat atacagaggg |
| 61 | agcttcctag ctgggatatt ggagcagcaa gaggctggga |
| | agccatcact taccttgcac |
| 121 | tgagaaagaa gacaaaggca agttgaaaag cggagaaata |
| | gtggcccagt ggttgaaaaa |
| 181 | ttgaagcaaa tgcaggaatt ctttgggctg aaagtgactg |
| | ggaaaccaga tgctgaaacc |
| 241 | ctgaaggtga tgaagcagcc cagatgtgga gtgcctgatg |
| | tggctcagtt tgtcctcact |
| 301 | gaggggaacc ctcgctggga gcaaacacat ctgacctaca |
| | ggattgaaaa ttacacgcca |
| 361 | gatttgccaa gagcagatgt ggaccatgcc attgagaaag |
| | ccttccaact ctggagtaat |
| 421 | gtcacacctc tgacattcac caaggtctct gagggtcaag |
| | cagacatcat gatatctttt |
| 481 | gtcaggggag atcatcggga caactctcct tttgatggac |
| | ctggaggaaa tcttgctcat |
| 541 | gcttttcaac caggcccagg tattggaggg gatgctcatt |
| | ttgatgaaga tgaaaggtgg |
| 601 | accaacaatt tcagagagta caacttacat cgtgttgcag |
| | ctcatgaact cggccattct |
| 661 | cttggactct cccattctac tgatatcggg gctttgatgt |
| | accctagcta caccttcagt |
| 721 | ggtgatgttc agctagctca ggatgacatt gatggcatcc |
| | aagccatata tggacgttcc |
| 781 | caaaatcctg tccagcccat cggcccacaa accccaaaag |
| | cgtgtgacag taagctaacc |
| 841 | tttgatgcta taactacgat tcggggagaa gtgatgttct |
| | ttaaagacag attctacatg |
| 901 | cgcacaaatc ccttctaccc ggaagttgag ctcaatttca |
| | tttctgtttt ctggccacaa |
| 961 | ctgccaaatg ggcttgaagc tgcttacgaa tttgccgaca |
| | gagatgaagt ccggtttttc |
| 1021 | aaagggaata agtactgggc tgttcaggga cagaatgtgc |
| | tacacggata ccccaaggac |
| 1081 | atctacagct cctttggctt ccctagaact gtgaagcata |
| | tcgatgctgc tctttctgag |
| 1141 | gaaaacactg gaaaaaccta cttctttgtt gctaacaaat |
| | actggaggta tgatgaatat |
| 1201 | aaacgatcta tggatccagg ttatcccaaa atgatagcac |
| | atgactttcc tggaattggc |
| 1261 | cacaaagttg atgcagtttt catgaaagat ggatttttct |
| | atttctttca tggaacaaga |
| 1321 | caatacaaat ttgatcctaa aacgaagaga attttgactc |
| | tccagaaagc taatagctgg |
| 1381 | ttcaactgca ggaaaaattg aacattacta atttgaatgg |
| | aaaacacatg gtgtgagtcc |
| 1441 | aaagaaggtg ttttcctgaa gaactgtcta ttttctcagt |
| | catttttaac ctctagagtc |
| 1501 | actgatacac agaatataat cttatttata cctcagtttg |
| | catatttttt tactatttag |
| 1561 | aatgtagccc tttttgtact gatataattt agttccacaa |
| | atggtgggta caaaaagtca |
| 1621 | agtttgtggc ttatggattc atataggcca gagttgcaaa |
| | gatcttttcc agagtatgca |
| 1681 | actctgacgt tgatcccaga gagcagcttc agtgacaaac |
| | atatcctttc aagacagaaa |
| 1741 | gagacaggag acatgagtct ttgccggagg aaaagcagct |
| | caagaacaca tgtgcagtca |
| 1801 | ctggtgtcac cctggatagg caagggataa ctcttctaac |
| | acaaaataag tgttttatgt |
| 1861 | ttggaataaa gtcaaccttg tttctactgt tttatacact |
| | ttc |

The amino acid sequence of human MMP-1 (interstitial collagenase isoform 2), provided by Genbank Accession No. NP_001139410.1, is incorporated herein by reference, and is shown below (SEQ ID NO: 29).

```
    1 mqeffglkvt gkpdaetlkv mkqprcgvpd vaqfvltegn prweqthlty rienytpdlp
  61 radvdhaiek afqlwsnvtp ltftkvsegq adimisfvrg dhrdnspfdg pggnlahafq
121 pgpgiggdah fdederwtnn freynlhrva ahelghslgl shstdigalm ypsytfsgdv
181 qlaqddidgi qaiygrsqnp vqpigpqtpk acdskltfda ittirgevmf fkdrfymrtn
241 pfypevelnf isvfwpqlpn gleaayefad rdevrffkgn kywavqgqnv lhgypkdiys
301 sfgfprtvkh idaalseent gktyffvank ywrydeykrs mdpgypkmia hdfpgighkv
361 davfmkdgff yffhgtrqyk fdpktkrilt lqkanswfnc rkn
```

The siRNA used to target human MMP-1 variant 1 mRNA include following sequences (SEQ ID NO: 30-33):

SEQ NO: 30:
5′-UUAGCUUACUGUCACACGC-3′
SEQ NO: 31:
5′-UUAUAUUCAUCAUACCUCC-3′
SEQ NO: 32:
5′-UUGUCUUCUUUCUCAGUGC-3′
SEQ NO: 33:
5′-UUCGUAAGCAGCUUCAAGC-3′

The molecular beacon used to target human MMP-1 variant 1 mRNA includes the following sequences (SEQ ID NO: 34-36):

SEQ NO 34:
5′-CCGGTC TTCGTAAGCAGCTTCAAGC GACCGG-3′
SEQ NO 35:
5′-CCGGTC TAAAGAACATCACTTTCC GACCGG-3′
SEQ NO 36:
5′-CCGGTC TAAAACAGTAGAAACAAGG GACCGG-3′

The mRNA transcript sequence encoding human MMP-9, provided by Genbank Accession No. NM_004994.2, is incorporated herein by reference, and is shown below (SEQ ID NO: 37).

```
      1 agacacctct gccctcacca tgagcctctg gcagcccctg gtcctggtgc tcctggtgct
    61 gggctgctgc tttgctgccc ccagacagcg ccagtccacc cttgtgctct tccctggaga
  121 cctgagaacc aatctcaccg acaggcagct ggcagaggaa tacctgtacc gctatggtta
  181 cactcgggtg gcagagatgc gtggagagtc gaaatctctg gggcctgcgc tgctgcttct
  241 ccagaagcaa ctgtccctgc ccgagaccgg tgagctggat agcgccacgc tgaaggccat
  301 gcgaacccca cggtgcgggg tcccagacct gggcagattc caaacctttg agggcgacct
  361 caagtggcac caccacaaca tcacctattg gatccaaaac tactcggaag acttgccgcg
  421 ggcggtgatt gacgacgcct ttgcccgcgc cttcgcactg tggagcgcgg tgacgccgct
  481 caccttcact cgcgtgtaca gccgggacgc agacatcgtc atccagtttg gtgtcgcgga
  541 gcacggagac gggtatccct tcgacgggaa ggacgggctc ctggcacacg cctttcctcc
  601 tggccccggc attcagggag acgcccattt cgacgatgac gagttgtggt ccctgggcaa
  661 gggcgtcgtg gttccaactc ggtttggaaa cgcagatggc gcggcctgcc acttcccctt
  721 catcttcgag ggccgctcct actctgcctg caccaccgac ggtcgctccg acggcttgcc
  781 ctggtgcagt accacggcca actacgacac cgacgaccgg tttggcttct gccccagcga
  841 gagactctac acccaggacg gcaatgctga tgggaaaccc tgccagtttc cattcatctt
  901 ccaaggccaa tcctactccg cctgcaccac ggacggtcgc tccgacggct accgctggtg
  961 cgccaccacc gccaactacg accgggacaa gctcttcggc ttctgcccga cccgagctga
1021 ctcgacggtg atggggggca actcggcggg ggagctgtgc gtcttcccct tcactttcct
1081 gggtaaggag tactcgacct gtaccagcga gggccgcgga gatgggcgcc tctggtgcgc
1141 taccacctcg aactttgaca gcgacaagaa gtggggcttc tgcccggacc aaggatacag
1201 tttgttcctc gtggcggcgc atgagttcgg ccacgcgctg ggcttagatc attcctcagt
1261 gccggaggcg ctcatgtacc ctatgtaccg cttcactgag gggcccccct tgcataagga
1321 cgacgtgaat ggcatccggc acctctatgg tcctcgccct gaacctgagc cacggcctcc
1381 aaccaccacc acaccgcagc ccacggctcc cccgacggtc tgccccaccg gaccccccac
1441 tgtccacccc tcagagcgcc ccacagctgg ccccacaggt cccccctcag ctggccccac
1501 aggtcccccc actgctggcc cttctacggc cactactgtg cctttgagtc cggtggacga
1561 tgcctgcaac gtgaacatct tcgacgccat cgcggagatt gggaaccagc tgtatttgtt
1621 caaggatggg aagtactggc gattctctga gggcaggggg agccggccgc agggcccctt
1681 ccttatcgcc gacaagtggc ccgcgctgcc ccgcaagctg gactcggtct ttgaggagcg
1741 gctctccaag aagcttttct tcttctctgg gcgccaggtg tgggtgtaca caggcgcgtc
1801 ggtgctgggc ccgaggcgtc tggacaagct gggcctggga gccgacgtgg cccaggtgac
1861 cggggccctc cggagtggca gggggaagat gctgctgttc agcgggcggc gcctctggag
1921 gttcgacgtg aaggcgcaga tggtggatcc ccggagcgcc agcgaggtgg accggatgtt
1981 ccccggggtg cctttggaca cgcacgacgt cttccagtac cgagagaaag cctatttctg
2041 ccaggaccgc ttctactggc gcgtgagttc ccggagtgag ttgaaccagg tggaccaagt
2101 gggctacgtg acctatgaca tcctgcagtg ccctgaggac tagggctccc gtcctgcttt
2161 ggcagtgcca tgtaaatccc cactgggacc aaccctgggg aaggagccag tttgccggat
2221 acaaactggt attctgttct ggaggaaagg gaggagtgga ggtgggctgg gccctctctt
2281 ctcacctttg ttttttgttg gagtgtttct aataaacttg gattctctaa cctttaaaaa
2341 aaaaaaaaaa aaaaaaaaaa aaaaaaaaaa aaaaaaaaaa aaaaaaa
```

The amino acid sequence of human MMP-9 (preproprotein), provided by Genbank Accession No. NP_004985.2, is incorporated herein by reference, and is shown below (SEQ ID NO: 38).

```
    1 mslwqplvlv llvlgccfaa prqrqstivl fpgdlrtnit drqlaeeyly rygytrvaem
  61 rgeskslgpa llllqkqlsl petgeldsat lkamrtprcg vpdlgrfqtf egdlkwhhhn
121 itywiqnyse dlpravidda farafalwsa vtpltftrvy srdadiviqf gvaehgdgyp
181 fdgkdgllah afppgpgiqg dahfdddelw slgkgvvvpt rfgnadgaac hfpfifegrs
241 ysacttdgrs dglpwcstta nydtddrfgf cpserlytqd gnadgkpcqf pfifqgqsys
301 acttdgrsdg yrwcattany drdklfgfcp tradstvmgg nsagelcvfp fifigkeyst
361 ctsegrgdgr lwcattsnfd sdkkwgfcpd qgyslflvaa hefghalgld hssvpealmy
421 pmyrftegpp lhkddvngir hlygprpepe prppttttpq ptapptvcpt gpptvhpser
481 ptagptgpps agptgpptag pstattvpls pvddacnvni fdaiaeignq lylfkdgkyw
541 rfsegrgsrp qgpfliadkw palprkldsv feerlskklf ffsgrqvwvy tgasvlgprr
601 ldklglgadv aqvtgalrsg rgkmllfsgr rlwrfdvkaq mvdprsasev drmfpgvpld
661 thdvfqyrek ayfcqdrfyw rvssrselnq vdqvgyvtyd ilqcped
(signal protein AA 1-19; proportein AA 20-707; mature protein 107-707)
```

The siRNA used to target human MMP-9 mRNA include following sequences (SEQ ID NO: 39-42):

SEQ NO: 39:
5′-UUGUCGCUGUCAAAGUUCGAG-3′
SEQ NO: 40:
5′-UUCUUGUCGCUGUCAAAGUUC-3′
SEQ NO: 41:
5′-UUCAACUCACUCCGGGAACUC-3′
SEQ NO: 42:
5′-UUCACGUCGUCCUUAUGCAAG-3′

The molecular beacon used to target human MMP-9 mRNA includes the following sequences (SEQ ID NO:43-45):

SEQ NO: 43:
5′-CCGGTC TTGTCGCTGTCAAAGTTCGGACCGG-3′
SEQ NO: 44:
5′-CCGGTC TTATTAGAAACACTCCAAC GACCGG-3′
SEQ NO: 45:
5′-CCGGTC ATTCACGTCGTCCTTATGC GACCGG-3′

The mRNA transcript sequence encoding human MMP-3, provided by Genbank Accession No. NM_002422.3, is incorporated herein by reference, and is shown below (SEQ ID NO: 46).

```
      1 ctacaaggag gcaggcaaga cagcaaggca tagagacaac atagagctaa gtaaagccag
    61 tggaaatgaa gagtcttcca atcctactgt tgctgtgcgt ggcagtttgc tcagcctatc
  121 cattggatgg agctgcaagg ggtgaggaca ccagcatgaa ccttgttcag aaatatctag
  181 aaaactacta cgacctcaaa aaagatgtga aacagtttgt taggagaaag gacagtggtc
  241 ctgttgttaa aaaaatccga gaaatgcaga agttccttgg attggaggtg acggggaagc
  301 tggactccga cactctggag gtgatgcgca agcccaggtg tggagttcct gatgttggtc
  361 acttcagaac ctttcctggc atcccgaagt ggaggaaaac ccaccttaca tacaggattg
  421 tgaattatac accagatttg ccaaaagatg ctgttgattc tgctgttgag aaagctctga
  481 aagtctggga agaggtgact ccactcacat tctccaggct gtatgaagga gaggctgata
  541 taatgatctc ttttgcagtt agagaacatg gagactttta cccttttgat ggacctggaa
  601 atgttttggc ccatgcctat gcccctgggc cagggattaa tggagatgcc cactttgatg
  661 atgatgaaca atggacaaag gatacaacag ggaccaattt atttctcgtt gctgctcatg
  721 aaattggcca ctccctgggt ctctttcact cagccaacac tgaagctttg atgtacccac
  781 tctatcactc actcacagac ctgactcggt tccgcctgtc tcaagatgat ataaatggca
  841 ttcagtccct ctatggacct ccccctgact cccctgagac ccccctggta cccacggaac
  901 ctgtccctcc agaacctggg acgccagcca actgtgatcc tgctttgtcc tttgatgctg
  961 tcagcactct gaggggagaa atcctgatct ttaaagacag gcacttttgg cgcaaatccc
1021 tcaggaagct tgaacctgaa ttgcatttga tctcttcatt ttggccatct cttccttcag
1081 gcgtggatgc cgcatatgaa gttactagca aggacctcgt tttcattttt aaaggaaatc
1141 aattctgggc tatcagagga aatgaggtac gagctggata cccaagaggc atccacaccc
1201 taggtttccc tccaaccgtg aggaaaatcg atgcagccat ttctgataag gaaaagaaca
1261 aaacatattt ctttgtagag gacaaatact ggagatttga tgagaagaga aattccatgg
1321 agccaggctt tcccaagcaa atagctgaag actttccagg gattgactca aagattgatg
1381 ctgtttttga agaatttggg ttcttttatt tctttactgg atcttcacag ttggagtttg
1441 acccaaatgc aaagaaagtg acacacactt tgaagagtaa cagctggctt aattgttgaa
1501 agagatatgt agaaggcaca atatgggcac tttaaatgaa gctaataatt cttcacctaa
1561 gtctctgtga attgaaatgt tcgttttctc ctgcctgtgc tgtgactcga gtcacactca
1621 agggaacttg agcgtgaatc tgtatcttgc cggtcatttt tatgttatta cagggcattc
1681 aaatgggctg ctgcttagct tgcaccttgt cacatagagt gatctttccc aagagaaggg
1741 gaagcactcg tgtgcaacag acaagtgact gtatctgtgt agactatttg cttatttaat
1801 aaagacgatt tgtcagttat tttatctt
```

The amino acid sequence of human MMP-3 (preproprotein), provided by Genbank Accession No. NP_002413.1, is incorporated herein by reference, and is shown below (SEQ ID NO: 47).

```
    1 mkslpillll cvavcsaypl dgaargedts mnlvqkylen yydlkkdvkq fvrrkdsgpv
  61 vkkiremqkf lglevtgkld sdtlevmrkp rcgvpdvghf rtfpgipkwr kthltyrivn
121 ytpdlpkdav dsavekalkv weevtpltfs rlyegeadim isfavrehgd fypfdgpgnv
181 lahayapgpg ingdahfddd eqwtkdttgt nlflvaahei ghslglfhsa ntealmyply
241 hsltdltrfr lsqddingiq slygpppdsp etplvptepv ppepgtpanc dpalsfdays
301 firgeilifk drhfwrkslr klepelhlis sfwpslpsgv daayevtskd lvfifkgnqf
361 wairgnevra gyprgihtlg fpptvrkida aisdkeknkt yffvedkywr fdekrnsmep
421 gfpkqiaedf pgidskidav feefgffyff tgssqlefdp nakkythtlk snswlnc
(signal peptide AA 1-17; proprotein AA 18-477; mature protein AA 100-477).
```

The siRNA used to target human MMP-3 mRNA include following sequences (SEQ ID NO: 48-51):

SEQ NO: 48:
5′-UUCAUCAUCAUCAAAGUGGG-3′
SEQ NO: 49:
5′-UAAUAACAUAAAAAUGACCG-3′
SEQ NO: 50:
5′-UAGUCUACACAGAUACAGUC-3′
SEQ NO: 51:
5′-UAUAUCAUCUUGAGACAGGC-3′

The molecular beacon used to target human MMP-3 mRNA includes the following sequences (SEQ ID NO: 52-54):

SEQ NO 52:
5′-CCGGTC TATATCATCTTGAGACAGGC GACCGG-3′
SEQ NO 53:
5′-CCGGTC TTTCTCTTCTCATCAAATCT GACCGG-3′
SEQ NO 54:
5′-CCGGTC TAACAAACTGTTTCACATCT GACCGG-3′

The mRNA transcript sequence encoding human IL-1 alpha, provided by Genbank Accession No. NM_000575.3, is incorporated herein by reference, and is shown below (SEQ ID NO: 55).

```
      1 accaggcaac accattgaag gctcatatgt aaaaatccat gccttccttt ctcccaatct
    61 ccattcccaa acttagccac tggcttctgg ctgaggcctt acgcatacct cccggggctt
  121 gcacacacct tcttctacag aagacacacc ttgggcatat cctacagaag accaggcttc
  181 tctctggtcc ttggtagagg gctactttac tgtaacaggg ccagggtgga gagttctctc
  241 ctgaagctcc atcccctcta taggaaatgt gttgacaata ttcagaagag taagaggatc
  301 aagacttctt tgtgctcaaa taccactgtt ctcttctcta ccctgcccta accaggagct
  361 tgtcacccca aactctgagg tgatttatgc cttaatcaag caaacttccc tcttcagaaa
  421 agatggctca ttttccctca aaagttgcca ggagctgcca agtattctgc caattcaccc
  481 tggagcacaa tcaacaaatt cagccagaac acaactacag ctactattag aactattatt
  541 attaataaat tcctctccaa atctagcccc ttgacttcgg atttcacgat ttctcccttc
  601 ctcctagaaa cttgataagt ttcccgcgct tccctttttc taagactaca tgtttgtcat
  661 cttataaagc aaaggggtga ataaatgaac caaatcaata acttctggaa tatctgcaaa
  721 caacaataat atcagctatg ccatctttca ctattttagc cagtatcgag ttgaatgaac
  781 atagaaaaat acaaaactga attcttccct gtaaattccc cgttttgacg acgcacttgt
  841 agccacgtag ccacgcctac ttaagacaat tacaaaaggc gaagaagact gactcaggct
  901 taagctgcca gccagagagg gagtcatttc attggcgttt gagtcagcaa agaagtcaag
  961 atggccaaag ttccagacat gtttgaagac ctgaagaact gttacagtga aaatgaagaa
1021 gacagttcct ccattgatca tctgtctctg aatcagaaat ccttctatca tgtaagctat
1081 ggcccactcc atgaaggctg catggatcaa tctgtgtctc tgagtatctc tgaaacctct
1141 aaaacatcca agcttacctt caaggagagc atggtggtag tagcaaccaa cgggaaggtt
1201 ctgaagaaga gacggttgag tttaagccaa tccatcactg atgatgacct ggaggccatc
1261 gccaatgact cagaggaaga aatcatcaag cctaggtcag caccttttag cttcctgagc
1321 aatgtgaaat acaactttat gaggatcatc aaatacgaat tcatcctgaa tgacgccctc
1381 aatcaaagta taattcgagc caatgatcag tacctcacgg ctgctgcatt acataatctg
1441 gatgaagcag tgaaatttga catgggtgct tataagtcat caaaggatga tgctaaaatt
1501 accgtgattc taagaatctc aaaaactcaa ttgtatgtga ctgcccaaga tgaagaccaa
1561 ccagtgctgc tgaaggagat gcctgagata cccaaaacca tcacaggtag tgagaccaac
1621 ctcctcttct tctgggaaac tcacggcact aagaactatt tcacatcagt tgcccatcca
1681 aacttgttta ttgccacaaa gcaagactac tgggtgtgct tggcaggggg gccaccctct
1741 atcactgact ttcagatact ggaaaaccag gcgtaggtct ggagtctcac ttgtctcact
1801 tgtgcagtgt tgacagttca tatgtaccat gtacatgaag aagctaaatc ctttactgtt
1861 agtcatttgc tgagcatgta ctgagccttg taattctaaa tgaatgttta cactctttgt
1921 aagagtggaa ccaacactaa catataatgt tgttatttaa agaacaccct atattttgca
1981 tagtaccaat cattttaatt attattcttc ataacaattt taggaggacc agagctactg
2041 actatggcta ccaaaaagac tctacccata ttacagatgg gcaaattaag gcataagaaa
2101 actaagaaat atgcacaata gcagttgaaa caagaagcca cagacctagg atttcatgat
2161 ttcatttcaa ctgtttgcct tctactttta agttgctgat gaactcttaa tcaaatagca
2221 taagtttctg ggacctcagt tttatcattt tcaaaatgga gggaataata cctaagcctt
2281 cctgccgcaa cagtttttta tgctaatcag ggaggtcatt ttggtaaaat acttcttgaa
2341 gccgagcctc aagatgaagg caaagcacga aatgttattt tttaattatt atttatatat
2401 gtatttataa atatatttaa gataattata atatactata tttatgggaa ccccttcatc
2461 ctctgagtgt gaccaggcat cctccacaat agcagacagt gttttctggg ataagtaagt
2521 ttgatttcat taatacaggg cattttggtc caagttgtgc ttatcccata gccaggaaac
2581 tctgcattct agtacttggg agacctgtaa tcatataata aatgtacatt aattaccttg
2641 agccagtaat tggtccgatc tttgactctt ttgccattaa acttacctgg gcattcttgt
2701 ttcaattcca cctgcaatca agtcctacaa gctaaaatta gatgaactca actttgacaa
2761 ccatgagacc actgttatca aaactttctt ttctggaatg taatcaatgt ttcttctagg
2821 ttctaaaaat tgtgatcaga ccataatgtt acattattat caacaatagt gattgataga
2881 gtgttatcag tcataactaa ataaagcttg caacaaaatt ctctgacaaa aaaaaaaaaa
2941 aaa
```

The amino acid sequence of human IL-1 alpha (proprotein), provided by Genbank Accession No. NP_000566.3, is incorporated herein by reference, and is shown below (SEQ ID NO: 56).

```
    1 makvpdmfed lkncysenee dsssidhlsl nqksfyhvsy gplhegcmdq syslsisets
  61 ktskltfkes mvvvatngkv lkkrrlslsq sitdddleai andseeeiik prsapfsfls
121 nvkynfmrii kyefilndal nqsiirandq yltaaalhnl deavkfdmga yksskddaki
181 tvilrisktq lyvtaqdedq pvllkempei pktitgsetn llffwethgt knyftsvahp
241 nlfiatkqdy wvclaggpps itdfqilenq a (mature peptide AA 113-271).
```

The siRNA used to target human IL-1 alpha mRNA include following sequences (SEQ ID NO: 57-60):

SEQ NO: 57:
5′-UUUCUAUGUUCAUUCAACUC-3′
SEQ NO: 58:
5′-UCAUUCAACUCGAUACUGGC-3′
SEQ NO: 59:
5′-UUCAUUCAACUCGAUACUGG-3′
SEQ NO: 60:
5′-UAAUAGUUCUAAUAGUAGCU-3′

The molecular beacon used to target human IL-1 alpha mRNA includes the following sequences (SEQ ID NO: 61-63):

SEQ NO 61:
5′-CCGGTC TTTCTTAGTTTTCTTATGCC GACCGG-3′
SEQ NO 62:
5′-CCGGTC TAATAGTTCTAATAGTAGC GACCGG-3′
SEQ NO 63:
5′-CCGGTC TATGAACTGTCAACACTGC GACCGG-3′

The mRNA transcript sequence encoding human IL-1 beta, provided by Genbank Accession No. NM_000576.2, is incorporated herein by reference, and is shown below (SEQ ID NO: 64).

```
      1 accaaacctc ttcgaggcac aaggcacaac aggctgctct gggattctct tcagccaatc
    61 ttcattgctc aagtgtctga agcagccatg gcagaagtac ctgagctcgc cagtgaaatg
  121 atggcttatt acagtggcaa tgaggatgac ttgttctttg aagctgatgg ccctaaacag
  181 atgaagtgct ccttccagga cctggacctc tgccctctgg atggcggcat ccagctacga
  241 atctccgacc accactacag caagggcttc aggcaggccg cgtcagttgt tgtggccatg
  301 gacaagctga ggaagatgct ggttccctgc ccacagacct tccaggagaa tgacctgagc
  361 accttctttc ccttcatctt tgaagaagaa cctatcttct tcgacacatg ggataacgag
  421 gcttatgtgc acgatgcacc tgtacgatca ctgaactgca cgctccggga ctcacagcaa
  481 aaaagcttgg tgatgtctgg tccatatgaa ctgaaagctc tccacctcca gggacaggat
  541 atggagcaac aagtggtgtt ctccatgtcc tttgtacaag gagaagaaag taatgacaaa
  601 atacctgtgg ccttgggcct caaggaaaag aatctgtacc tgtcctgcgt gttgaaagat
  661 gataagccca ctctacagct ggagagtgta gatcccaaaa attacccaaa gaagaagatg
  721 gaaaagcgat ttgtcttcaa caagatagaa atcaataaca agctggaatt tgagtctgcc
  781 cagttcccca actggtacat cagcacctct caagcagaaa acatgcccgt cttcctggga
  841 gggaccaaag gcggccagga tataactgac ttcaccatgc aatttgtgtc ttcctaaaga
  901 gagctgtacc cagagagtcc tgtgctgaat gtggactcaa tccctagggc tggcagaaag
  961 ggaacagaaa ggtttttgag tacggctata gcctggactt tcctgttgtc tacaccaatg
1021 cccaactgcc tgccttaggg tagtgctaag aggatctcct gtccatcagc caggacagtc
1081 agctctctcc tttcagggcc aatccccagc ccttttgttg agccaggcct ctctcacctc
1141 tcctactcac ttaaagcccg cctgacagaa accacggcca catttggttc taagaaaccc
1201 tctgtcattc gctcccacat tctgatgagc aaccgcttcc ctatttattt atttatttgt
1261 ttgtttgttt tattcattgg tctaatttat tcaaaggggg caagaagtag cagtgtctgt
1321 aaaagagcct agtttttaat agctatggaa tcaattcaat ttggactggt gtgctctctt
1381 taaatcaagt cctttaatta agactgaaaa tatataagct cagattattt aaatgggaat
1441 atttataaat gagcaaatat catactgttc aatggttctg aaataaactt cactgaag
```

The amino acid sequence of human IL-1 beta (proprotein), provided by Genbank Accession No. NP_000567.1, is incorporated herein by reference, and is shown below (SEQ ID NO: 65).

```
    1 maevpelase mmayysgned dlffeadgpk qmkcsfqdld lcpldggiql risdhhyskg
  61 frqaasvvva mdklrkmlvp cpqtfqendl stffpfifee epiffdtwdn eayvhdapvr
121 slnctlrdsq qkslvmsgpy elkalhlqgq dmeqqvvfsm sfvqgeesnd kipvalglke
181 knlylscvlk ddkptlqles vdpknypkkk mekrfvfnki einnklefes aqfpnwyist
241 sqaenmpvfl ggtkggqdit dftmqfvss (mature peptide AA 117-269)
```

The siRNA used to target human IL-1 beta mRNA includes the following sequences (SEQ ID NO: 66-69):

SEQ NO: 66:
5′-UUAUCAUCUUUCAACACGCAG-3′
SEQ NO: 67:
5′-UUUUACAGACACUGCUACUUC-3′
SEQ NO: 68:
5′-UUUGUCAUUACUUUCUUCUCC-3′
SEQ NO: 69:
5′-UACAGACACUGCUACUUCUUG-3′

The molecular beacon used to target human IL-1 beta mRNA includes the following sequences (SEQ ID NO: 70-72):

SEQ NO: 70:
5′-CCGGTC TTTTGTCATTACTTTCTTCTC GACCGG-3′
SEQ NO: 71:
5′-CCGGTC TTTCAGTCTTAATTAAAGGAC GACCGG-3′
SEQ NO: 72:
5′-CCGGTC TTACATAAATTAACTCAGCT GACCGG-3′

The mRNA transcript sequence encoding human IL-6, provided by Genbank Accession No. NM_000600.3, is incorporated herein by reference, and is shown below (SEQ ID NO: 73).

```
      1 aatattagag tctcaacccc caataaatat aggactggag atgtctgagg ctcattctgc
    61 cctcgagccc accgggaacg aaagagaagc tctatctccc ctccaggagc ccagctatga
  121 actccttctc cacaagcgcc ttcggtccag ttgccttctc cctggggctg ctcctggtgt
  181 tgcctgctgc cttccctgcc ccagtacccc caggagaaga ttccaaagat gtagccgccc
  241 cacacagaca gccactcacc tcttcagaac gaattgacaa acaaattcgg tacatcctcg
  301 acggcatctc agccctgaga aaggagacat gtaacaagag taacatgtgt gaaagcagca
  361 aagaggcact ggcagaaaac aacctgaacc ttccaaagat ggctgaaaaa gatggatgct
  421 tccaatctgg attcaatgag gagacttgcc tggtgaaaat catcactggt cttttggagt
  481 ttgaggtata cctagagtac ctccagaaca gatttgagag tagtgaggaa caagccagag
  541 ctgtgcagat gagtacaaaa gtcctgatcc agttcctgca gaaaaaggca aagaatctag
  601 atgcaataac cacccctgac ccaaccacaa atgccagcct gctgacgaag ctgcaggcac
  661 agaaccagtg gctgcaggac atgacaactc atctcattct gcgcagcttt aaggagttcc
  721 tgcagtccag cctgagggct cttcggcaaa tgtagcatgg gcacctcaga ttgttgttgt
  781 taatgggcat tccttcttct ggtcagaaac ctgtccactg ggcacagaac ttatgttgtt
  841 ctctatggag aactaaaagt atgagcgtta ggacactatt ttaattattt ttaatttatt
  901 aatatttaaa tatgtgaagc tgagttaatt tatgtaagtc atatttatat ttttaagaag
  961 taccacttga aacattttat gtattagttt tgaaataata atggaaagtg gctatgcagt
1021 ttgaatatcc tttgtttcag agccagatca tttcttggaa agtgtaggct tacctcaaat
1081 aaatggctaa cttatacata tttttaaaga aatatttata ttgtatttat ataatgtata
1141 aatggttttt ataccaataa atggcatttt aaaaaattca gcaaaaaaaa aaaaaaaaaa
1201 a
```

The amino acid sequence of human IL-6 (precursor), provided by Genbank Accession No. NP_000591.1, is incorporated herein by reference, and is shown below (SEQ ID NO: 74).

```
    1 mnsfstsafg pvafslglll vlpaafpapv ppgedskdva
        aphrqpltss eridkqiryi
  61 ldgisalrke tcnksnmces skealaennl nlpkmaekdg
        cfqsgfneet clvkiitgll
121 efevyleylq nrfesseeqa ravqmstkvl iqflqkkakn
        ldaittpdpt tnaslltklq
181 aqnqwlqdmt thlilrsfke flqsslralr qm
        (Signal peptide AA 1-29; mature peptide
        AA 30-212).
```

The siRNA used to target human IL-6 mRNA include following sequences (SEQ ID NO: 75-78):

SEQ NO: 75:
5′-UAAAAUAGUGUCCUAACGCUC-3′
SEQ NO: 76:
5′-UCACUACUCUCAAAUCUGUUC-3′
SEQ NO: 77:
5′-UUACUCUUGUUACAUGUCUCC-3′
SEQ NO: 78:
5′-UAACGCUCAUACUUUUAGUUC-3′

The molecular beacon used to target human IL-6 mRNA includes the following sequences (SEQ ID NO: 79-81):

SEQ NO 79:
5′-CCGGTC TTACTCTTGTTACATGTCYCC GACCTT-3′
SEQ NO 80:
5′-CCGGTC TTACTCTTGTTACATGTCTCC GACCTT-3′
SEQ NO 81:
5′-CCGGTC TACATAAAATGTTTCAAGTGG GACCTT-3′

The mRNA transcript sequence encoding human IL-8, provided by Genbank Accession No. NM_000584.3, is incorporated herein by reference, and is shown below (SEQ ID NO: 82).

```
      1 gagggtgcat aagttctcta gtagggtgat gatataaaaa gccaccggag cactccataa
    61 ggcacaaact ttcagagaca gcagagcaca caagcttcta ggacaagagc caggaagaaa
  121 ccaccggaag gaaccatctc actgtgtgta aacatgactt ccaagctggc cgtggctctc
  181 ttggcagcct tcctgatttc tgcagctctg tgtgaaggtg cagttttgcc aaggagtgct
  241 aaagaactta gatgtcagtg cataaagaca tactccaaac ctttccaccc caaatttatc
  301 aaagaactga gagtgattga gagtggacca cactgcgcca acacagaaat tattgtaaag
  361 ctttctgatg gaagagagct ctgtctggac cccaaggaaa actgggtgca gagggttgtg
  421 gagaagtttt tgaagagggc tgagaattca taaaaaaatt cattctctgt ggtatccaag
  481 aatcagtgaa gatgccagtg aaacttcaag caaatctact tcaacacttc atgtattgtg
  541 tgggtctgtt gtagggttgc cagatgcaat acaagattcc tggttaaatt tgaatttcag
  601 taaacaatga atagtttttc attgtaccat gaaatatcca gaacatactt atatgtaaag
  661 tattatttat ttgaatctac aaaaaacaac aaataatttt taaatataag gattttccta
  721 gatattgcac gggagaatat acaaatagca aaattgaggc caagggccaa gagaatatcc
  781 gaactttaat ttcaggaatt gaatgggttt gctagaatgt gatatttgaa gcatcacata
  841 aaaatgatgg gacaataaat tttgccataa agtcaaattt agctggaaat cctggatttt
  901 tttctgttaa atctggcaac cctagtctgc tagccaggat ccacaagtcc ttgttccact
  961 gtgccttggt ttctccttta tttctaagtg gaaaaagtat tagccaccat cttacctcac
1021 agtgatgttg tgaggacatg tggaagcact ttaagttttt tcatcataac ataaattatt
1081 ttcaagtgta acttattaac ctatttatta tttatgtatt tatttaagca tcaaatattt
1141 gtgcaagaat ttggaaaaat agaagatgaa tcattgattg aatagttata aagatgttat
1201 agtaaattta ttttatttta gatattaaat gatgttttat tagataaatt tcaatcaggg
1261 tttttagatt aaacaaacaa acaattgggt acccagttaa attttcattt cagataaaca
1321 acaaataatt ttttagtata agtacattat tgtttatctg aaattttaat tgaactaaca
1381 atcctagttt gatactccca gtcttgtcat tgccagctgt gttggtagtg ctgtgttgaa
1441 ttacggaata atgagttaga actattaaaa cagccaaaac tccacagtca atattagtaa
1501 tttcttgctg gttgaaactt gtttattatg tacaaataga ttcttataat attatttaaa
1561 tgactgcatt tttaaataca aggctttata tttttaactt taagatgttt ttatgtgctc
1621 tccaaatttt ttttactgtt tctgattgta tggaaatata aaagtaaata tgaaacattt
1681 aaaatataat ttgttgtcaa agtaaaaaaa aaaaaaaa
```

The amino acid sequence of human IL-8(precursor), provided by Genbank Accession No. NP_000575.1, is incorporated herein by reference, and is shown below (SEQ ID NO: 83).

```
  1 mtsklavall aaflisaalc egavlprsak elrcqcikty
      skpfhpkfik elrviesgph
61 canteiivkl sdgrelcldp kenwvqrvve kflkraens
```

The siRNA used to target human IL-8 mRNA include following sequences (SEQ ID NO: 84-87):

SEQ NO: 84:
5′-UUUGUUUAAUCUAAAAACCC-3′
SEQ NO: 85:
5′-UUUACACACAGUGAGAUGGU-3′
SEQ NO: 86:
5′-UUCAAAUAUCACAUUCUAGC-3′
SEQ NO: 87:
5′-UUAUGCACUGACAUCUAAGU-3′

The molecular beacon used to target human IL-8 mRNA includes the following sequences (SEQ ID NO: 88-90):

SEQ NO 88:
5′-CCGGTC TATCACATTCTAGCAAACCC GACCGG-3′
SEQ NO 89:
5′-CCGGTC TACTAGAGAACTTATGCACC GACCGG-3′
SEQ NO 90:
5′-CCGGTC TAGTTCTAACTCATTATTCC GACCGG-3′

The mRNA transcript sequence encoding human IL-1R type 1 variant 1, provided by Genbank Accession No. NM_000877.3, is incorporated herein by reference, and is shown below (SEQ ID NO: 91).

```
      1 gtggccggcg gccggagccg actcggagcg cgcggcgccg gccgggagga gccggagagc
    61 ggccgggccg ggcggtgggg gcgccggcct gccccgcgcg ccccagggag cggcaggaat
  121 gtgacaatcg cgcgcccgcg caccgaagca ctcctcgctc ggctcctagg gctctcgccc
  181 ctctgagctg agccgggttc cgcccggggc tgggatccca tcaccctcca cggccgtccg
  241 tccaggtaga cgcaccctct gaagatggtg actccctcct gagaagctgg accccttggt
  301 aaaagacaag gccttctcca agaagaatat gaaagtgtta ctcagactta tttgtttcat
  361 agctctactg atttcttctc tggaggctga taaatgcaag gaacgtgaag aaaaaataat
  421 tttagtgtca tctgcaaatg aaattgatgt tcgtccctgt cctcttaacc caaatgaaca
  481 caaaggcact ataacttggt ataaagatga cagcaagaca cctgtatcta cagaacaagc
  541 ctccaggatt catcaacaca aagagaaact ttggtttgtt cctgctaagg tggaggattc
  601 aggacattac tattgcgtgg taagaaattc atcttactgc ctcagaatta aaataagtgc
  661 aaaatttgtg gagaatgagc ctaacttatg ttataatgca caagccatat ttaagcagaa
  721 actacccgtt gcaggagacg gaggacttgt gtgcccttat atggagtttt ttaaaaatga
  781 aaataatgag ttacctaaat tacagtggta taaggattgc aaacctctac ttcttgacaa
  841 tatacacttt agtggagtca aagataggct catcgtgatg aatgtggctg aaaagcatag
  901 agggaactat acttgtcatg catcctacac atacttgggc aagcaatatc ctattacccg
  961 ggtaatagaa tttattactc tagaggaaaa caaacccaca aggcctgtga ttgtgagccc
1021 agctaatgag acaatggaag tagacttggg atcccagata caattgatct gtaatgtcac
1081 cggccagttg agtgacattg cttactggaa gtggaatggg tcagtaattg atgaagatga
1141 cccagtgcta ggggaagact attacagtgt ggaaaatcct gcaaacaaaa gaaggagtac
1201 cctcatcaca gtgcttaata tatcggaaat tgaaagtaga ttttataaac atccatttac
1261 ctgttttgcc aagaatacac atggtataga tgcagcatat atccagttaa tatatccagt
1321 cactaatttc cagaagcaca tgattggtat atgtgtcacg ttgacagtca taattgtgtg
1381 ttctgttttc atctataaaa tcttcaagat tgacattgtg cifiggtaca gggattcctg
1441 ctatgatttt ctcccaataa aagcttcaga tggaaagacc tatgacgcat atatactgta
1501 tccaaagact gttggggaag ggtctacctc tgactgtgat atttttgtgt ttaaagtctt
1561 gcctgaggtc ttggaaaaac agtgtggata taagctgttc atttatggaa gggatgacta
1621 cgttggggaa gacattgttg aggtcattaa tgaaaacgta aagaaaagca gaagactgat
1681 tatcatttta gtcagagaaa catcaggctt cagctggctg ggtggttcat ctgaagagca
1741 aatagccatg tataatgctc ttgttcagga tggaattaaa gttgtcctgc ttgagctgga
1801 gaaaatccaa gactatgaga aaatgccaga atcgattaaa ttcattaagc agaaacatgg
1861 ggctatccgc tggtcagggg actttacaca gggaccacag tctgcaaaga caaggttctg
1921 gaagaatgtc aggtaccaca tgccagtcca gcgacggtca ccttcatcta aacaccagtt
1981 actgtcacca gccactaagg agaaactgca aagagaggct cacgtgcctc tcgggtagca
2041 tggagaagtt gccaagagtt ctttaggtgc ctcctgtctt atggcgttgc aggccaggtt
2101 atgcctcatg ctgacttgca gagttcatgg aatgtaacta tatcatcctt tatccctgag
2161 gtcacctgga atcagattat taagggaata agccatgacg tcaatagcag cccagggcac
2221 ttcagagtag agggcttggg aagatctttt aaaaaggcag taggcccggt gtggtggctc
2281 acgcctataa tcccagcact ttgggaggct gaagtgggtg gatcaccaga ggtcaggagt
2341 tcgagaccag cccagccaac atggcaaaac cccatctcta ctaaaaatac aaaaatgagc
2401 taggcatggt ggcacacgcc tgtaatccca gctacacctg aggctgaggc aggagaattg
2461 cttgaaccgg ggagacggag gttgcagtga gccgagtttg ggccactgca ctctagcctg
2521 gcaacagagc aagactccgt ctcaaaaaaa gggcaataaa tgccctctct gaatgtttga
2581 actgccaaga aaaggcatgg agacagcgaa ctagaagaaa gggcaagaag gaaatagcca
2641 ccgtctacag atggcttagt taagtcatcc acagcccaag ggcggggcta tgccttgtct
2701 ggggaccctg tagagtcact gaccctggag cggctctcct gagaggtgct gcaggcaaag
2761 tgagactgac acctcactga ggaagggaga catattcttg gagaactttc catctgcttg
2821 tattttccat acacatcccc agccagaagt tagtgtccga agaccgaatt ttattttaca
2881 gagcttgaaa actcacttca atgaacaaag ggattctcca ggattccaaa gttttgaagt
2941 catcttagct ttccacagga gggagagaac ttaaaaaagc aacagtagca gggaattgat
3001 ccacttctta atgctttcct ccctggcatg accatcctgt cctttgttat tatcctgcat
3061 tttacgtctt tggaggaaca gctccctagt ggcttcctcc gtctgcaatg tcccttgcac
3121 agcccacaca tgaaccatcc ttcccatgat gccgctcttc tgtcatcccg ctcctgctga
3181 aacacctccc aggggctcca cctgttcagg agctgaagcc catgctttcc caccagcatg
3241 tcactcccag accacctccc tgccctgtcc tccagcttcc cctcgctgtc ctgctgtgtg
3301 aattcccagg ttggcctggt ggccatgtcg cctgccccca gcactcctct gtctctgctc
3361 ttgcctgcac ccttcctcct cctttgccta ggaggccttc tcgcattttc tctagctgat
3421 cagaatttta ccaaaattca gaacatcctc caattccaca gtctctggga gactttccct
3481 aagaggcgac ttcctctcca gccttctctc tctggtcagg cccactgcag agatggtggt
3541 gagcacatct gggaggctgg tctccctcca gctggaattg ctgctctctg agggagaggc
3601 tgtggtggct gtctctgtcc ctcactgcct tccaggagca atttgcacat gtaacataga
3661 tttatgtaat gctttatgtt taaaaacatt ccccaattat cttatttaat ttttgcaatt
3721 attctaattt tatatataga gaaagtgacc tattttttaa aaaaatcaca ctctaagttc
3781 tattgaacct aggacttgag cctccatttc tggcttctag tctggtgttc tgagtacttg
3841 atttcaggtc aataacggtc ccccctcact ccacactggc acgtttgtga gaagaaatga
3901 cattttgcta ggaagtgacc gagtctagga atgcttttat tcaagacacc aaattccaaa
3961 cttctaaatg ttggaatttt caaaaattgt gtttagattt tatgaaaaac tcttctactt
4021 tcatctattc tttccctaga ggcaaacatt tcttaaaatg tttcattttc attaaaaatg
4081 aaagccaaat ttatatgcca ccgattgcag gacacaagca cagttttaag agttgtatga
4141 acatggagag gacttttggt ttttatattt ctcgtattta atatgggtga acaccaactt
4201 ttatttggaa taataatttt cctcctaaac aaaaacacat tgagtttaag tctctgactc
4261 ttgcctttcc acctgctttc tcctgggccc gctttgcctg cttgaaggaa cagtgctgtt
4321 ctggagctgc tgttccaaca gacagggcct agctttcatt tgacacacag actacagcca
4381 gaagcccatg gagcagggat gtcacgtctt gaaaagccta ttagatgttt tacaaattta
4441 attttgcaga ttattttagt ctgtcatcca gaaaatgtgt cagcatgcat agtgctaaga
4501 aagcaagcca atttggaaac ttaggttagt gacaaaattg gccagagagt gggggtgatg
4561 atgaccaaga attacaagta gaatggcagc tggaatttaa ggagggacaa gaatcaatgg
4621 ataagcgtgg gtggaggaag atccaaacag aaaagtgcaa agttattccc catcttccaa
4681 gggttgaatt ctggaggaag aagacacatt cctagttccc cgtgaacttc ctttgactta
4741 ttgtccccac taaaacaaaa caaaaaactt ttaatgcctt ccacattaat tagattttct
4801 tgcagttttt ttatggcatt tttttaaaga tgccctaagt gttgaagaag agtttgcaaa
4861 tgcaacaaaa tatttaatta ccggttgtta aaactggttt agcacaattt atattttccc
4921 tctcttgcct ttcttatttg caataaaagg tattgagcca ttttttaaat gacatttttg
4981 ataaattatg tttgtactag ttgatgaagg agtttttttt aacctgttta tataattttg
5041 cagcagaagc caaatttttt gtatattaaa gcaccaaatt catgtacagc atgcatcacg
5101 gatcaataga ctgtacttat tttccaataa aattttcaaa ctttgtactg ttaaaaaaaa
5161 aaaaaaaaaa
```

The amino acid sequence of human IL-1R type 1 isoform 1 precursor, provided by Genbank Accession No. NP_000868.1, is incorporated herein by reference, and is shown below (SEQ ID NO: 92).

```
    1 mkvllrlicf iallisslea dkckereeki ilvssaneid
        vrpcpinpne hkgtitwykd
  61 dsktpvsteq asrihqhkek lwfvpakved sghyycvvrn
        ssyclrikis akfvenepnl
121 cynaqaifkq klpvagdggl vcpymeffkn ennelpklqw
        ykdckpllld nihfsgvkdr
181 livmnvaekh rgnytchasy tylgkqypit rviefitlee
        nkptrpvivs panetmevdl
241 gsqiqlicnv tgqlsdiayw kwngsvided dpvlgedyys
        venpankrrs tlitvinise
301 iesrfykhpf tcfaknthgi daayiqliyp vtnfqkhmig
        icvtltviiv csvfiykifk
361 idivlwyrds cydflpikas dgktydayil ypktvgegst
        sdcdifvfkv 1pevlekqcg
421 yklfiygrdd yvgedivevi nenvkksrrl iiilvretsg
        fswlggssee qiamynalvq
481 dgikvvllel ekiqdyekmp esikfikqkh gairwsgdft
        qgpqsaktrf wknvryhmpv
541 qrrspsskhq llspatkekl qreahvplg
        (Signal peptide 1-20; mature peptide
        AA 21-569).
```

The siRNA used to target human IL-1R type 1 variant 1 mRNA include following sequences (SEQ ID NO: 93-96):

SEQ NO: 93:
5′-UUUCUUCUCACAAACGUGCC-3′
SEQ NO: 94:
5′-UUAUACCAAGUUAUAGUGCC-3′
SEQ NO: 95:
5′-UUGUAAAACAUCUAAUAGGC-3′
SEQ NO: 96:
5′-UUUCCACACUGUAAUAGUCU-3′

The molecular beacon used to target human IL-1R type 1 variant 1 mRNA includes the following sequences (SEQ ID NO: 97-99):

SEQ NO 97:
5′-CCGGTC TTTCTTCTCACAAACGTGC GACCGG-3′
SEQ NO 98:
5′-CCGGTC TTAAACACAAAAATATCAC GACCGG-3′
SEQ NO 99:
5′-CCGGTC TTTCCACACTGTAATAGTC GACCGG-3′

The mRNA transcript sequence encoding human TNF-alpha, provided by Genbank Accession No. NM_000594.3, is incorporated herein by reference, and is shown below (SEQ ID NO: 100).

| | |
|---|---|
| 1 | cagacgctcc ctcagcaagg acagcagagg accagctaag agggagagaa gcaactacag |
| 61 | accccccctg aaaacaaccc tcagacgcca catcccctga caagctgcca ggcaggttct |
| 121 | cttcctctca catactgacc cacggctcca ccctctctcc cctggaaagg acaccatgag |
| 181 | cactgaaagc atgatccggg acgtggagct ggccgaggag gcgctcccca agaagacagg |
| 241 | ggggccccag ggctccaggc ggtgcttgtt cctcagcctc ttctccttcc tgatcgtggc |
| 301 | aggcgccacc acgctcttct gcctgctgca ctttggagtg atcggccccc agagggaaga |
| 361 | gttccccagg gacctctctc taatcagccc tctggcccag gcagtcagat catcttctcg |
| 421 | aaccccgagt gacaagcctg tagcccatgt tgtagcaaac cctcaagctg aggggcagct |
| 481 | ccagtggctg aaccgccggg ccaatgccct cctggccaat ggcgtggagc tgagagataa |
| 541 | ccagctggtg gtgccatcag agggcctgta cctcatctac tcccaggtcc tcttcaaggg |
| 601 | ccaaggctgc ccctccaccc atgtgctcct cacccacacc atcagccgca tcgccgtctc |
| 661 | ctaccagacc aaggtcaacc tcctctctgc catcaagagc ccctgccaga gggagacccc |
| 721 | agagggggct gaggccaagc cctggtatga gcccatctat ctgggagggg tcttccagct |
| 781 | ggagaagggt gaccgactca gcgctgagat caatcggccc gactatctcg actttgccga |
| 841 | gtctgggcag gtctactttg ggatcattgc cctgtgagga ggacgaacat ccaaccttcc |
| 901 | caaacgcctc ccctgcccca atccctttat taccccctcc ttcagacacc ctcaacctct |
| 961 | tctggctcaa aaagagaatt gggggcttag ggtcggaacc caagcttaga actttaagca |
| 1021 | acaagaccac cacttcgaaa cctgggattc aggaatgtgt ggcctgcaca gtgaagtgct |
| 1081 | ggcaaccact aagaattcaa actggggcct ccagaactca ctggggccta cagctttgat |
| 1141 | ccctgacatc tggaatctgg agaccaggga gcctttggtt ctggccagaa tgctgcagga |
| 1201 | cttgagaaga cctcacctag aaattgacac aagtggacct taggccttcc tctctccaga |
| 1261 | tgtttccaga cttccttgag acacggagcc cagccctccc catggagcca gctccctcta |
| 1321 | tttatgtttg cacttgtgat tatttattat ttatttatta tttatttatt tacagatgaa |
| 1381 | tgtatttatt tgggagaccg gggtatcctg ggggacccaa tgtaggagct gccttggctc |
| 1441 | agacatgttt tccgtgaaaa cggagctgaa caataggctg ttcccatgta gccccctggc |
| 1501 | ctctgtgcct tcttttgatt atgtttttta aaatatttat ctgattaagt tgtctaaaca |
| 1561 | atgctgattt ggtgaccaac tgtcactcat tgctgagcct ctgctcccca ggggagttgt |
| 1621 | gtctgtaatc gccctactat tcagtggcga gaaataaagt ttgcttagaa aagaaaaaaa |
| 1681 | aaaaaa |

The amino acid sequence of human TNF-alpha, provided by Genbank Accession No. NP_000585.2, is incorporated herein by reference, and is shown below (SEQ ID NO: 101).

| | |
|---|---|
| 1 | mstesmirdv elaeealpkk tggpqgsrrc lflslfsfli vagattlfcl lhfgvigpqr |
| 61 | eefprdlsli splaqavrss srtpsdkpva hvvanpqaeg qlqwlnrran allangvelr |
| 121 | dnqlvvpseg lyliysqvlf kgqgcpsthv llthtisria vsyqtkvnll saikspcqre |
| 181 | tpegaeakpw yepiylggvf qlekgdrlsa einrpdyldf aesgqvyfgi ial (Exemplary regions |
| | include residues 36-56 (transmembrane region; residue 80-glycosylation site; 102- |
| | 233 tumor necrosis factor site; and residues 105, 106, 111, 153, 160 and 165- |
| | receptor binding sites) |

The siRNA used to target human TNF-alpha mRNA include following sequences (SEQ ID NO: 102-105):

SEQ NO: 102:
5′-AAUAAAUAAUCACAAGUGC-3′
SEQ NO: 103:
5′-UAAAAAACAUAAUCAAAAG-3′
SEQ NO: 104:
5′-UAAUAAAUAAUCACAAGUG-3′
SEQ NO: 105:
5′-UUUUCUUUUCUAAGCAAAC-3′

The molecular beacon used to target human TNF-alpha mRNA includes the following sequences (SEQ ID NO: 106-108):

SEQ NO 106:
5′-CCGGTC AAACATAATCAAAAGAAGG GACCGG-3′
SEQ NO 107:
5′-CCGGTC TAAAAAACATAATCAAAAG GACCGG-3′
SEQ NO 108:
5′-CCGGTC TATTTTAAAAAACATAATC GACCGG-3′

The mRNA transcript sequence encoding human VEGF A variant 1, provided by Genbank Accession No. NM_001025366.2, is incorporated herein by reference, and is shown below (SEQ ID NO: 109).

| | |
|---|---|
| 1 | tcgcggaggc ttggggcagc cgggtagctc ggaggtcgtg gcgctggggg ctagcaccag |
| 61 | cgctctgtcg ggaggcgcag cggttaggtg gaccggtcag cggactcacc ggccagggcg |
| 121 | ctcggtgctg gaatttgata ttcattgatc cgggttttat ccctcttctt ttttcttaaa |
| 181 | catttttttt taaaactgta ttgtttctcg ttttaattta tttttgcttg ccattcccca |
| 241 | cttgaatcgg gccgacggct tggggagatt gctctacttc cccaaatcac tgtggatttt |
| 301 | ggaaaccagc agaaagagga aagaggtagc aagagctcca gagagaagtc gaggaagaga |
| 361 | gagacggggt cagagagagc gcgcgggcgt gcgagcagcg aaagcgacag gggcaaagtg |
| 421 | agtgacctgc ttttgggggt gaccgccgga gcgcggcgtg agccctcccc cttgggatcc |
| 481 | cgcagctgac cagtcgcgct gacggacaga cagacagaca ccgcccccag ccccagctac |
| 541 | cacctcctcc ccggccggcg gcggacagtg gacgcggcgg cgagccgcgg gcaggggccg |
| 601 | gagcccgcgc ccggaggcgg ggtggagggg gtcggggctc gcggcgtcgc actgaaactt |
| 661 | ttcgtccaac ttctgggctg ttctcgcttc ggaggagccg tggtccgcgc gggggaagcc |
| 721 | gagccgagcg gagccgcgag aagtgctagc tcgggccggg aggagccgca gccggaggag |
| 781 | ggggaggagg aagaagagaa ggaagaggag agggggccgc agtggcgact cggcgctcgg |
| 841 | aagccgggct catggacggg tgaggcggcg gtgtgcgcag acagtgctcc agccgcgcgc |
| 901 | gctccccagg ccctggcccg ggcctcgggc cggggaggaa gagtagctcg ccgaggcgcc |
| 961 | gaggagagcg ggccgcccca cagcccgagc cggagaggga gcgcgagccg cgccggcccc |
| 1021 | ggtcgggcct ccgaaaccat gaactttctg ctgtcttggg tgcattggag ccttgccttg |
| 1081 | ctgctctacc tccaccatgc caagtggtcc caggctgcac ccatggcaga aggaggaggg |
| 1141 | cagaatcatc acgaagtggt gaagttcatg gatgtctatc agcgcagcta ctgccatcca |
| 1201 | atcgagaccc tggtggacat cttccaggag taccctgatg agatcgagta catcttcaag |
| 1261 | ccatcctgtg tgcccctgat gcgatgcggg ggctgctgca atgacgaggg cctggagtgt |
| 1321 | gtgcccactg aggagtccaa catcaccatg cagattatgc ggatcaaacc tcaccaaggc |
| 1381 | cagcacatag gagagatgag cttcctacag cacaacaaat gtgaatgcag accaaagaaa |
| 1441 | gatagagcaa gacaagaaaa aaaatcagtt cgaggaaagg gaaaggggca aaaacgaaag |
| 1501 | cgcaagaaat cccggtataa gtcctggagc gtgtacgttg gtgcccgctg ctgtctaatg |
| 1561 | ccctggagcc tccctggccc ccatccctgt gggccttgct cagagcggag aaagcatttg |
| 1621 | tttgtacaag atccgcagac gtgtaaatgt tcctgcaaaa acacagactc gcgttgcaag |
| 1681 | gcgaggcagc ttgagttaaa cgaacgtact tgcagatgtg acaagccgag gcggtgagcc |
| 1741 | gggcaggagg aaggagcctc cctcagggtt tcgggaacca gatctctcac caggaaagac |
| 1801 | tgatacagaa cgatcgatac agaaaccacg ctgccgccac cacaccatca ccatcgacag |
| 1861 | aacagtcctt aatccagaaa cctgaaatga aggaagagga gactctgcgc agagcacttt |
| 1921 | gggtccggag ggcgagactc cggcggaagc attcccgggc gggtgaccca gcacggtccc |
| 1981 | tcttggaatt ggattcgcca ttttattttt cttgctgcta aatcaccgag cccggaagat |
| 2041 | tagagagttt tatttctggg attcctgtag acacacccac ccacatacat acatttatat |
| 2101 | atatatatat tatatatata taaaaataaa tatctctatt ttatatatat aaaatatata |
| 2161 | tattcttttt ttaaattaac agtgctaatg ttattggtgt cttcactgga tgtatttgac |
| 2221 | tgctgtggac ttgagttggg aggggaatgt tcccactcag atcctgacag ggaagaggag |
| 2281 | gagatgagag actctggcat gatctttttt ttgtcccact tggtggggcc agggtcctct |
| 2341 | cccctgccca ggaatgtgca aggccagggc atgggggcaa atatgaccca gttttgggaa |
| 2401 | caccgacaaa cccagccctg gcgctgagcc tctctacccc aggtcagacg gacagaaaga |
| 2461 | cagatcacag gtacagggat gaggacaccg gctctgacca ggagtttggg gagcttcagg |
| 2521 | acattgctgt gctttgggga ttccctccac atgctgcacg cgcatctcgc ccccaggggc |
| 2581 | actgcctgga agattcagga gcctgggcgg ccttcgctta ctctcacctg cttctgagtt |
| 2641 | gcccaggaga ccactggcag atgtcccggc gaagagaaga gacacattgt tggaagaagc |
| 2701 | agcccatgac agctcccctt cctgggactc gccctcatcc tcttcctgct ccccttcctg |
| 2761 | gggtgcagcc taaaaggacc tatgtcctca caccattgaa accactagtt ctgtcccccc |
| 2821 | aggagacctg gttgtgtgtg tgtgagtggt tgaccttcct ccatcccctg gtccttccct |
| 2881 | tcccttcccg aggcacagag agacagggca ggatccacgt gcccattgtg gaggcagaga |
| 2941 | aaagagaaag tgttttatat acggtactta tttaatatcc ctttttaatt agaaattaaa |
| 3001 | acagttaatt taattaaaga gtagggtttt ttttcagtat tcttggttaa tatttaattt |
| 3061 | caactattta tgagatgtat cttttgctct ctcttgctct cttatttgta ccggtttttg |
| 3121 | tatataaaat tcatgtttcc aatctctctc tccctgatcg gtgacagtca ctagcttatc |
| 3181 | ttgaacagat atttaatttt gctaacactc agctctgccc tccccgatcc cctggctccc |
| 3241 | cagcacacat tcctttgaaa taaggtttca atatacatct acatactata tatatatttg |
| 3301 | gcaacttgta tttgtgtgta tatatatata tatatgttta tgtatatatg tgattctgat |
| 3361 | aaaatagaca ttgctattct gttttttata tgtaaaaaca aaacaagaaa aaatagagaa |
| 3421 | ttctacatac taaatctctc tcctttttta attttaatat ttgttatcat ttatttattg |
| 3481 | gtgctactgt ttatccgtaa taattgtggg gaaaagatat taacatcacg tctttgtctc |
| 3541 | tagtgcagtt tttcgagata ttccgtagta catatttatt tttaaacaac gacaaagaaa |
| 3601 | tacagatata tcttaaaaaa aaaaaagcat tttgtattaa agaatttaat tctgatctca |
| 3661 | aaaaaaaaaa aaaaaaa |

The amino acid sequence of human VEGF A isoform 1, provided by Genbank Accession No. NP_001020537.2, is incorporated herein by reference, and is shown below (SEQ ID NO: 110).

| | |
|---|---|
| 1 | mtdrqtdtap spsyhllpgr rrtvdaaasr gqgpepapgg gvegvgargv alklfvqllg |
| 61 | csrfggavvr ageaepsgaa rsassgreep qpeegeeeee keeergpqwr lgarkpgswt |
| 121 | geaavcadsa paarapqala rasgrggrva rrgaeesgpp hspsrrgsas ragpgraset |
| 181 | mnfllswvhw slalllylhh akwsqaapma egggqnhhev vkfmdvyqrs ychpietlvd |
| 241 | ifqeypdeie yifkpscvpl mrcggccnde glecvptees nitmqimrik phqgqhigem |
| 301 | sflqhnkcec rpkkdrarqe kksvrgkgkg qkrkrkksry kswsvyvgar cclmpwslpg |
| 361 | phpcgpcser rkhlfvqdpq tckcsckntd srckarqlel nertcrcdkp rr |

The siRNA used to target human VEGF A variant 1 mRNA include following sequences (SEQ ID NO: 111-114):

SEQ NO: 111:
5′-UAAAACUCUCUAAUCUUCCGG-3′
SEQ NO: 112:
5′-UUCCUUCUCUUCUUCCUCCUC-3′
SEQ NO: 113:
5′-UAUACACACAAAUACAAGUUG-3′
SEQ NO: 114:
5′-UUAAAACGAGAAACAAUACAG-3′

The molecular beacon used to target human VEGF A variant 1 mRNA includes the following sequences (SEQ ID NO: 115-117):

SEQ NO 115:
5′-CCGGTC TAAAACTCTCTAATCTTCC GACCGG-3′
SEQ NO 116:
5′-CCGGTC TTTGATCCGCATAATCTGC GACCGG-3′
SEQ NO 117:
5′-CCGGTC TTGAAATTAAATATTAACC GACCGG-3′

The mRNA transcript sequence encoding human TGF-beta 1, provided by Genbank Accession No. NM_000660.5, is incorporated herein by reference, and is shown below (SEQ ID NO: 118).

| | |
|---|---|
| 1 | agccggtccc cgccgccgcc gcccttcgcg ccctgggcca tctccctccc acctccctcc |
| 61 | gcggagcagc cagacagcga gggccccggc cgggggcagg ggggacgccc cgtccggggc |
| 121 | acccccccgg ctctgagccg cccgcggggc cggcctcggc ccggagcgga ggaaggagtc |
| 181 | gccgaggagc agcctgaggc cccagagtct gagacgagcc gccgccgccc ccgccactgc |
| 241 | ggggaggagg gggaggagga gcgggaggag ggacgagctg gtcgggagaa gaggaaaaaa |
| 301 | acttttgaga cttttccgtt gccgctggga gccggaggcg cggggacctc ttggcgcgac |
| 361 | gctgccccgc gaggaggcag gacttgggga ccccagaccg cctccctttg ccgccgggga |
| 421 | cgcttgctcc ctccctgccc cctacacggc gtccctcagg cgcccccatt ccggaccagc |
| 481 | cctcgggagt cgccgacccg gcctcccgca aagacttttc cccagacctc gggcgcaccc |
| 541 | cctgcacgcc gccttcatcc ccggcctgtc tcctgagccc ccgcgcatcc tagacccttt |
| 601 | ctcctccagg agacggatct ctctccgacc tgccacagat cccctattca agaccaccca |
| 661 | ccttctggta ccagatcgcg cccatctagg ttatttccgt gggatactga gacacccccg |
| 721 | gtccaagcct cccctccacc actgcgccct tctccctgag gacctcagct ttccctcgag |
| 781 | gccctcctac cttttgccgg gagaccccca gcccctgcag gggcggggcc tccccaccac |
| 841 | accagccctg ttcgcgctct cggcagtgcc ggggggcgcc gcctccccca tgccgccctc |
| 901 | cgggctgcgg ctgctgccgc tgctgctacc gctgctgtgg ctactggtgc tgacgcctgg |
| 961 | ccggccggcc gcgggactat ccacctgcaa gactatcgac atggagctgg tgaagcggaa |
| 1021 | gcgcatcgag gccatccgcg gccagatcct gtccaagctg cggctcgcca gccccccgag |
| 1081 | ccagggggag gtgccgcccg gcccgctgcc cgaggccgtg ctcgccctgt acaacagcac |
| 1141 | ccgcgaccgg gtggccgggg agagtgcaga accggagccc gagcctgagg ccgactacta |
| 1201 | cgccaaggag gtcacccgcg tgctaatggt ggaaacccac aacgaaatct atgacaagtt |
| 1261 | caagcagagt acacacagca tatatatgtt cttcaacaca tcagagctcc gagaagcggt |
| 1321 | acctgaaccc gtgttgctct cccgggcaga gctgcgtctg ctgaggctca agttaaaagt |
| 1381 | ggagcagcac gtggagctgt accagaaata cagcaacaat tcctggcgat acctcagcaa |
| 1441 | ccggctgctg gcacccagcg actcgccaga gtggttatct tttgatgtca ccggagttgt |
| 1501 | gcggcagtgg ttgagccgtg gaggggaaat tgagggcttt cgccttagcg cccactgctc |
| 1561 | ctgtgacagc agggataaca cactgcaagt ggacatcaac gggttcacta ccggccgccg |
| 1621 | aggtgacctg gccaccattc atggcatgaa ccggcctttc ctgcttctca tggccacccc |
| 1681 | gctggagagg gcccagcatc tgcaaagctc ccggcaccgc cgagccctgg acaccaacta |
| 1741 | ttgcttcagc tccacggaga agaactgctg cgtgcggcag ctgtacattg acttccgcaa |
| 1801 | ggacctcggc tggaagtgga tccacgagcc caagggctac catgccaact tctgcctcgg |
| 1861 | gccctgcccc tacatttgga gcctggacac gcagtacagc aaggtcctgg ccctgtacaa |
| 1921 | ccagcataac ccgggcgcct cggcggcgcc gtgctgcgtg ccgcaggcgc tggagccgct |
| 1981 | gcccatcgtg tactacgtgg gccgcaagcc caaggtggag cagctgtcca acatgatcgt |
| 2041 | gcgctcctgc aagtgcagct gaggtcccgc cccgccccgc cccgccccgg caggcccggc |
| 2101 | cccaccccgc cccgcccccg ctgccttgcc catgggggct gtatttaagg acacccgtgc |
| 2161 | cccaagccca cctggggccc cattaaagat ggagagagga ctgcggatct ctgtgtcatt |
| 2221 | gggcgcctgc ctggggtctc catccctgac gttcccccac tcccactccc tctctctccc |
| 2281 | tctctgcctc ctcctgcctg tctgcactat tcctttgccc ggcatcaagg cacaggggac |
| 2341 | cagtggggaa cactactgta gttagatcta tttattgagc accttgggca ctgttgaagt |
| 2401 | gccttacatt aatgaactca ttcagtcacc atagcaacac tctgagatgc agggactctg |
| 2461 | ataacaccca ttttaaaggt gaggaaacaa gcccagagag gttaagggag gagttcctgc |
| 2521 | ccaccaggaa cctgctttag tgggggatag tgaagaagac aataaaagat agtagttcag |
| 2581 | gcc |

The amino acid sequence of human TGF-beta 1 (precursor), provided by Genbank Accession No. NP_000651.3, is incorporated herein by reference, and is shown below (SEQ ID NO:119).

| | |
|---|---|
| 1 | mppsglrllp lllpllwllv ltpgrpaagl stcktidmel vkrkrieair gqilsklrla |
| 61 | sppsqgevpp gplpeavlal ynstrdrvag esaepepepe adyyakevtr vlmvethnei |
| 121 | ydkfkqsths iymffntsel reavpepvll sraelrllrl klkveqhvel yqkysnnswr |
| 181 | ylsnrllaps dspewlsfdv tgvvrqwlsr ggeiegfrls ahcscdsrdn tlqvdingft |
| 241 | tgrrgdlati hgmnrpflll matpleraqh lqssrhrral dtnycfsste knccvrqlyi |
| 301 | dfrkdlgwkw ihepkgyhan fclgpcpyiw sldtqyskvl alynqhnpga saapccvpqa |
| 361 | leplpivyyv grkpkveqls nmivrsckcs (Signal peptide AA 1-29; mature |
| | peptide AA 30-278). |

The siRNA used to target human TGF-beta 1 mRNA include following sequences (SEQ ID NO: 120-123):

SEQ NO: 120:
5′-UAUUGUCUUCUUCACUAUC-3′
SEQ NO: 121:
5′-UAGAUCUAACUACAGUAGU-3′
SEQ NO: 122:
5′-UAUAUGCUGUGUGUACUCU-3′
SEQ NO: 123:
5′-UAUAUAUGCUGUGUGUACU-3′

The molecular beacon used to target human TGF-beta 1 mRNA includes the following sequences (SEQ ID NO: 124-126):

SEQ NO 124:
5′-CCGGTC ATATATGCTGTGTGTACTC GACCGG-3′
SEQ NO 125:
5′-CCGGTC TTTTATTGTCTTCTTCACT GACCGG-3′
SEQ NO 126:
5′-CCGGTC TATATATGCTGTGTGTACT GACCGG-3′

The mRNA transcript sequence encoding human TGF-beta 2 variant 1, provided by Genbank Accession No. NM_001135599.2, is incorporated herein by reference, and is shown below (SEQ ID NO: 127).

| | |
|---|---|
| 1 | gtgatgttat ctgctggcag cagaaggttc gctccgagcg gagctccaga agctcctgac |
| 61 | aagagaaaga cagattgaga tagagataga aagagaaaga gagaaagaga cagcagagcg |
| 121 | agagcgcaag tgaaagaggc aggggagggg gatggagaat attagcctga cggtctaggg |
| 181 | agtcatccag gaacaaactg aggggctgcc cggctgcaga caggaggaga cagagaggat |
| 241 | ctattttagg gtggcaagtg cctacctacc ctaagcgagc aattccacgt tggggagaag |
| 301 | ccagcagagg ttgggaaagg gtgggagtcc aagggagccc ctgcgcaacc ccctcaggaa |
| 361 | taaaactccc cagccagggt gtcgcaaggg ctgccgttgt gatccgcagg gggtgaacgc |
| 421 | aaccgcgacg gctgatcgtc tgtggctggg ttggcgtttg gagcaagaga aggaggagca |
| 481 | ggagaaggag ggagctggag gctggaagcg tttgcaagcg gcggcggcag caacgtggag |
| 541 | taaccaagcg ggtcagcgcg cgcccgccag ggtgtaggcc acggagcgca gctcccagag |
| 601 | caggatccgc gccgcctcag cagcctctgc ggcccctgcg gcacccgacc gagtaccgag |
| 661 | cgccctgcga agcgcaccct cctccccgcg gtgcgctggg ctcgccccca gcgcgcgcac |
| 721 | acgcacacac acacacacac acacacacgc acgcacacac gtgtgcgctt ctctgctccg |
| 781 | gagctgctgc tgctcctgct ctcagcgccg cagtggaagg caggaccgaa ccgctccttc |
| 841 | tttaaatata taaatttcag cccaggtcag cctcggcggc ccccctcacc gcgctcccgg |
| 901 | cgcccctccc gtcagttcgc cagctgccag ccccgggacc ttttcatctc ttcccttttg |
| 961 | gccggaggag ccgagttcag atccgccact ccgcacccga gactgacaca ctgaactcca |
| 1021 | cttcctcctc ttaaatttat ttctacttaa tagccactcg tctctttttt tccccatctc |
| 1081 | attgctccaa gaattttttt cttcttactc gccaaagtca gggttccctc tgcccgtccc |
| 1141 | gtattaatat ttccactttt ggaactactg gccttttctt tttaaaggaa ttcaagcagg |
| 1201 | atacgttttt ctgttgggca ttgactagat tgtttgcaaa agtttcgcat caaaaacaac |
| 1261 | aacaacaaaa aaccaaacaa ctctccttga tctatacttt gagaattgtt gatttctttt |
| 1321 | ttttattctg acttttaaaa acaacttttt tttccacttt tttaaaaaat gcactactgt |
| 1381 | gtgctgagcg cttttctgat cctgcatctg gtcacggtcg cgctcagcct gtctacctgc |
| 1441 | agcacactcg atatggacca gttcatgcgc aagaggatcg aggcgatccg cgggcagatc |
| 1501 | ctgagcaagc tgaagctcac cagtccccca gaagactatc ctgagcccga ggaagtcccc |
| 1561 | ccggaggtga tttccatcta caacagcacc agggacttgc tccaggagaa ggcgagccgg |
| 1621 | agggcggccg cctgcgagcg cgagaggagc gacgaagagt actacgccaa ggaggtttac |
| 1681 | aaaatagaca tgccgccctt cttcccctcc gaaactgtct gcccagttgt tacaacaccc |
| 1741 | tctggctcag tgggcagctt gtgctccaga cagtcccagg tgctctgtgg gtaccttgat |
| 1801 | gccatcccgc ccactttcta cagaccctac ttcagaattg ttcgatttga cgtctcagca |
| 1861 | atggagaaga atgcttccaa tttggtgaaa gcagagttca gagtctttcg tttgcagaac |
| 1921 | ccaaaagcca gagtgcctga acaacggatt gagctatatc agattctcaa gtccaaagat |
| 1981 | ttaacatctc caacccagcg ctacatcgac agcaaagttg tgaaaacaag agcagaaggc |
| 2041 | gaatggctct ccttcgatgt aactgatgct gttcatgaat ggcttcacca taaagacagg |
| 2101 | aacctgggat ttaaaataag cttacactgt ccctgctgca cttttgtacc atctaataat |
| 2161 | tacatcatcc caaataaaag tgaagaacta gaagcaagat ttgcaggtat tgatggcacc |
| 2221 | tccacatata ccagtggtga tcagaaaact ataaagtcca ctaggaaaaa aaacagtggg |
| 2281 | aagaccccac atctcctgct aatgttattg ccctcctaca gacttgagtc acaacagacc |
| 2341 | aaccggcgga agaagcgtgc tttggatgcg gcctattgct ttagaaatgt gcaggataat |
| 2401 | tgctgcctac gtccacttta cattgatttc aagagggatc tagggtggaa atggatacac |
| 2461 | gaacccaaag ggtacaatgc caacttctgt gctggagcat gcccgtattt atggagttca |
| 2521 | gacactcagc acagcagggt cctgagctta tataatacca taaatccaga agcatctgct |
| 2581 | tctccttgct gcgtgtccca agatttagaa cctctaacca ttctctacta cattggcaaa |
| 2641 | acacccaaga ttgaacagct ttctaatatg attgtaaagt cttgcaaatg cagctaaaat |
| 2701 | tcttggaaaa gtggcaagac caaaatgaca atgatgatga taatgatgat gacgacgaca |
| 2761 | acgatgatgc ttgtaacaag aaaacataag agagccttgg ttcatcagtg ttaaaaaatt |
| 2821 | tttgaaaagg cggtactagt tcagacactt tggaagtttg tgttctgttt gttaaaactg |
| 2881 | gcatctgaca caaaaaaagt tgaaggcctt attctacatt tcacctactt tgtaagtgag |
| 2941 | agagacaaga agcaaatttt ttttaaagaa aaaaataaac actggaagaa tttattagtg |
| 3001 | ttaattatgt gaacaacgac aacaacaaca acaacaacaa acaggaaaat cccattaagt |
| 3061 | ggagttgctg tacgtaccgt tcctatcccg cgcctcactt gatttttctg tattgctatg |
| 3121 | caataggcac ccttcccatt cttactctta gagttaacag tgagttattt attgtgtgtt |
| 3181 | actatataat gaacgtttca ttgcccttgg aaaataaaac aggtgtataa agtggagacc |
| 3241 | aaatactttg ccagaaactc atggatggct taaggaactt gaactcaaac gagccagaaa |
| 3301 | aaaagaggtc atattaatgg gatgaaaacc caagtgagtt attatatgac cgagaaagtc |
| 3361 | tgcattaaga taaagaccct gaaaacacat gttatgtatc agctgcctaa ggaagcttct |
| 3421 | tgtaaggtcc aaaaactaaa aagactgtta ataaaagaaa ctttcagtca gaataagtct |
| 3481 | gtaagttttt ttttttcttt ttaattgtaa atggttcttt gtcagtttag taaaccagtg |
| 3541 | aaatgttgaa atgttttgac atgtactggt caaacttcag accttaaaat attgctgtat |
| 3601 | agctatgcta taggtttttt cctttgtttt ggtatatgta accataccta tattattaaa |
| 3661 | atagatggat atagaagcca gcataattga aaacacatct gcagatctct tttgcaaact |
| 3721 | attaaatcaa aacattaact actttatgtg taatgtgtaa atttttacca tattttttat |
| 3781 | attctgtaat aatgtcaact atgatttaga ttgacttaaa tttgggctct ttttaatgat |
| 3841 | cactcacaaa tgtatgtttc ttttagctgg ccagtacttt tgagtaaagc ccctatagtt |
| 3901 | tgacttgcac tacaaatgca tttttttttt aataacattt gccctacttg tgctttgtgt |
| 3961 | ttctttcatt attatgacat aagctacctg ggtccacttg tcttttcttt tttttgtttc |
| 4021 | acagaaaaga tgggttcgag ttcagtggtc ttcatcttcc aagcatcatt actaaccaag |
| 4081 | tcagacgtta acaaattttt atgttaggaa aaggaggaat gttatagata catagaaaat |
| 4141 | tgaagtaaaa tgttttcatt ttagcaagga tttagggttc taactaaaac tcagaatctt |
| 4201 | tattgagtta agaaaagttt ctctaccttg gtttaatcaa tatttttgta aaatcctatt |
| 4261 | gttattacaa agaggacact tcataggaaa catctttttc tttagtcagg tttttaatat |
| 4321 | tcagggggaa attgaaagat atatatttta gtcgattttt caaaagggga aaaaagtcca |
| 4381 | ggtcagcata agtcattttg tgtatttcac tgaagttata aggtttttat aaatgttctt |
| 4441 | tgaaggggaa aaggcacaag ccaatttttc ctatgatcaa aaaattcttt ctttcctctg |
| 4501 | agtgagagtt atctatatct gaggctaaag tttaccttgc tttaataaat aatttgccac |
| 4561 | atcattgcag aagaggtatc ctcatgctgg ggttaataga atatgtcagt ttatcacttg |
| 4621 | tcgcttattt agctttaaaa taaaaattaa taggcaaagc aatggaatat ttgcagtttc |
| 4681 | acctaaagag cagcataagg aggcgggaat ccaaagtgaa gttgtttgat atggtctact |
| 4741 | tcttttttgg aatttcctga ccattaatta aagaattgga tttgcaagtt tgaaaactgg |
| 4801 | aaaagcaaga gatgggatgc cataatagta aacagccctt gtgttggatg taacccaatc |
| 4861 | ccagatttga gtgtgtgttg attatttttt tgtcttccac ttttctatta tgtgtaaatc |
| 4921 | acttttattt ctgcagacat tttcctctca gataggatga cattttgttt tgtattattt |
| 4981 | tgtctttcct catgaatgca ctgataatat tttaaatgct ctattttaag atctcttgaa |
| 5041 | tctgtttttt ttttttttaa tttgggggtt ctgtaaggtc tttatttccc ataagtaaat |
| 5101 | attgccatgg gaggggggtg gaggtggcaa ggaaggggtg aagtgctagt atgcaagtgg |
| 5161 | gcagcaatta tttttgtgtt aatcagcagt acaatttgat cgttggcatg gttaaaaaat |
| 5221 | ggaatataag attagctgtt ttgtattttg atgaccaatt acgctgtatt ttaacacgat |
| 5281 | gtatgtctgt ttttgtggtg ctctagtggt aaataaatta tttcgatgat atgtggatgt |
| 5341 | ctttttccta tcagtaccat catcgagtct agaaaacacc tgtgatgcaa taagactatc |
| 5401 | tcaagctgga aaagtcatac cacctttccg attgccctct gtgctttctc ccttaaggac |
| 5461 | agtcacttca gaagtcatgc tttaaagcac aagagtcagg ccatatccat caaggataga |
| 5521 | agaaatccct gtgccgtctt tttattccct tatttattgc tatttggtaa ttgtttgaga |
| 5581 | tttagtttcc atccagcttg actgccgacc agaaaaaatg cagagagatg tttgcaccat |
| 5641 | gctttggctt tctggttcta tgttctgcca acgccagggc caaaagaact ggtctagaca |
| 5701 | gtatcccctg tagccccata acttggatag ttgctgagcc agccagatat aacaagagcc |
| 5761 | acgtgctttc tggggttggt tgtttgggat cagctacttg cctgtcagtt tcactggtac |
| 5821 | cactgcacca caaacaaaaa aacccaccct atttcctcca atttttttgg ctgctaccta |
| 5881 | caagaccaga ctcctcaaac gagttgccaa tctcttaata aataggatta ataaaaaaag |
| 5941 | taattgtgac tcaaaaaaaa aaaaaa |

The amino acid sequence of human TGF-beta 2 isoform 1 precursor, provided by Genbank Accession No. NP_001129071.1, is incorporated herein by reference, and is shown below (SEQ ID NO: 128).

| | |
|---|---|
| 1 | mhycvlsafl ilhlvtvals lstcstldmd qfmrkrieai rgqilsklkl tsppedypep |
| 61 | eevppevisi ynstrdllqe kasrraaace rersdeeyya kevykidmpp ffpsetvcpv |
| 121 | vttpsgsvgs lcsrqsqvlc gyldaipptf yrpyfrivrf dvsameknas nlvkaefrvf |
| 181 | rlqnpkarvp eqrielyqil kskdltsptq ryidskvvkt raegewlsfd vtdavhewlh |
| 241 | hkdrnlgfki slhcpcctfv psnnyiipnk seelearfag idgtstytsg dqktikstrk |
| 301 | knsgktphll lmllpsyrle sqqtnrrkkr aldaaycfrn vqdncclrpl yidfkrdlgw |
| 361 | kwihepkgyn anfcagacpy lwssdtqhsr vlslyntinp easaspccvs qdlepltily |
| 421 | yigktpkieq lsnmivksck cs |

The siRNA used to target human TGF-beta 2 variant 1 mRNA include following sequences (SEQ ID NO: 129-132):

SEQ NO: 129:
5′-UAUCUCUAUCUCAAUCUGUC-3′
SEQ NO: 130:
5′-UUCUAUCUCUAUCUCAAUCU-3′
SEQ NO: 131:
5′-UUCUCUUUCUAUCUCUAUCU-3′
SEQ NO: 132:
5′-UCUAUCUCUAUCUCAAUCUG-3′

The molecular beacon used to target human TGF-beta 2 variant 1 mRNA includes the following sequences (SEQ ID NO: 133-135):

SEQ NO 133:
5′-CCGGTC TTCTATCTCTATCTCAATC GACCGG-3′
SEQ NO 134:
5′-CCGGTC TATCTCTATCTCAATCTGT GACCGG-3′
SEQ NO 135:
5′-CCGGTC TTCTCTTTCTATCTCTATC GACCGG-3′

The mRNA transcript sequence encoding human IGF-1 variant 4, provided by Genbank Accession No. NM_000618.3, is incorporated herein by reference, and is shown below (SEQ ID NO: 136).

| | |
|---|---|
| 1 | ttttgtagat aaatgtgagg attttctcta aatccctctt ctgtttgcta aatctcactg |
| 61 | tcactgctaa attcagagca gatagagcct gcgcaatgga ataaagtcct caaaattgaa |
| 121 | atgtgacatt gctctcaaca tctcccatct ctctggattt ctttttgctt cattattcct |
| 181 | gctaaccaat tcattttcag actttgtact tcagaagcaa tgggaaaaat cagcagtctt |
| 241 | ccaacccaat tatttaagtg ctgcttttgt gatttcttga aggtgaagat gcacaccatg |
| 301 | tcctcctcgc atctcttcta cctggcgctg tgcctgctca ccttcaccag ctctgccacg |
| 361 | gctggaccgg agacgctctg cggggctgag ctggtggatg ctcttcagtt cgtgtgtgga |
| 421 | gacaggggct tttatttcaa caagcccaca gggtatggct ccagcagtcg gagggcgcct |
| 481 | cagacaggca tcgtggatga gtgctgcttc cggagctgtg atctaaggag gctggagatg |
| 541 | tattgcgcac ccctcaagcc tgccaagtca gctcgctctg tccgtgccca gcgccacacc |
| 601 | gacatgccca agacccagaa ggaagtacat ttgaagaacg caagtagagg gagtgcagga |
| 661 | aacaagaact acaggatgta ggaagaccct cctgaggagt gaagagtgac atgccaccgc |
| 721 | aggatccttt gctctgcacg agttacctgt taaactttgg aacacctacc aaaaaataag |
| 781 | tttgataaca tttaaaagat gggcgtttcc cccaatgaaa tacacaagta aacattccaa |
| 841 | cattgtcttt aggagtgatt tgcaccttgc aaaaatggtc ctggagttgg tagattgctg |
| 901 | ttgatctttt atcaataatg ttctatagaa aagaaaaaaa aaatatatat atatatatat |
| 961 | cttagtccct gcctctcaag agccacaaat gcatgggtgt tgtatagatc cagttgcact |
| 1021 | aaattcctct ctgaatcttg gctgctggag ccattcattc agcaaccttg tctaagtggt |
| 1081 | ttatgaattg tttccttatt tgcacttctt tctacacaac tcgggctgtt tgttttacag |
| 1141 | tgtctgataa tcttgttagt ctatacccac cacctccctt cataaccttt atatttgccg |
| 1201 | aatttggcct cctcaaaagc agcagcaagt cgtcaagaag cacaccaatt ctaacccaca |
| 1261 | agattccatc tgtggcattt gtaccaaata taagttggat gcattttatt ttagacacaa |
| 1321 | agctttattt ttccacatca tgcttacaaa aaagaataat gcaaatagtt gcaactttga |
| 1381 | ggccaatcat ttttaggcat atgttttaaa catagaaagt ttcttcaact caaaagagtt |
| 1441 | ccttcaaatg atgagttaat gtgcaaccta attagtaact ttcctctttt tattttttcc |
| 1501 | atatagagca ctatgtaaat ttagcatatc aattatacag gatatatcaa acagtatgta |
| 1561 | aaactctgtt ttttagtata atggtgctat tttgtagttt gttatatgaa agagtctggc |
| 1621 | caaaacggta atacgtgaaa gcaaaacaat aggggaagcc tggagccaaa gatgacacaa |
| 1681 | ggggaagggt actgaaaaca ccatccattt gggaaagaag gcaaagtccc cccagttatg |
| 1741 | ccttccaaga ggaacttcag acacaaaagt ccactgatgc aaattggact ggcgagtcca |
| 1801 | gagaggaaac tgtggaatgg aaaaagcaga aggctaggaa ttttagcagt cctggtttct |
| 1861 | ttttctcatg gaagaaatga acatctgcca gctgtgtcat ggactcacca ctgtgtgacc |
| 1921 | ttgggcaagt cacttcacct ctctgtgcct cagtttcctc atctgcaaaa tgggggcaat |
| 1981 | atgtcatcta cctacctcaa aggggtggta taaggtttaa aaagataaag attcagattt |
| 2041 | tttttaccct gggttgctgt aagggtgcaa catcagggcg cttgagttgc tgagatgcaa |
| 2101 | ggaattctat aaataaccca ttcatagcat agctagagat tggtgaattg aatgctcctg |
| 2161 | acatctcagt tcttgtcagt gaagctatcc aaataactgg ccaactagtt gttaaaagct |
| 2221 | aacagctcaa tctcttaaaa cacttttcaa aatatgtggg aagcatttga ttttcaattt |
| 2281 | gattttgaat tctgcatttg gttttatgaa tacaaagata agtgaaaaga gagaaaggaa |
| 2341 | aagaaaaagg agaaaaacaa agagatttct accagtgaaa ggggaattaa ttactctttg |
| 2401 | ttagcactca ctgactcttc tatgcagtta ctacatatct agtaaaacct cgtttaatac |
| 2461 | tataaataat attctattca ttttgaaaaa cacaatgatt ccttcttttc taggcaatat |
| 2521 | aaggaaagtg atccaaaatt tgaaatatta aaataatatc taataaaaag tcacaaagtt |
| 2581 | atcttcttta acaaacttta ctcttattct tagctgtata tacatttttt taaaagtttg |
| 2641 | ttaaaatatg cttgactaga gtttccagtt gaaaggcaaa aacttccatc acaacaagaa |
| 2701 | atttcccatg cctgctcaga agggtagccc ctagctctct gtgaatgtgt tttatccatt |
| 2761 | caactgaaaa ttggtatcaa gaaagtccac tggttagtgt actagtccat catagcctag |
| 2821 | aaaatgatcc ctatctgcag atcaagattt tctcattaga acaatgaatt atccagcatt |
| 2881 | cagatctttc tagtcacctt agaacttttt ggttaaaagt acccaggctt gattatttca |
| 2941 | tgcaaattct atattttaca ttcttggaaa gtctatatga aaaacaaaaa taacatcttc |
| 3001 | agtttttctc ccactgggtc acctcaagga tcagaggcca ggaaaaaaaa aaaaaagact |
| 3061 | ccctggatct ctgaatatat gcaaaaagaa ggccccattt agtggagcca gcaatcctgt |
| 3121 | tcagtcaaca agtattttaa ctctcagtcc aacattattt gaattgagca cctcaagcat |
| 3181 | gcttagcaat gttctaatca ctatggacag atgtaaaaga aactatacat catttttgcc |
| 3241 | ctctgcctgt tttccagaca tacaggttct gtggaataag atactggact cctcttccca |
| 3301 | agatggcact tctttttatt tcttgtcccc agtgtgtacc ttttaaaatt attccctctc |
| 3361 | aacaaaactt tataggcagt cttctgcaga cttaacgtgt tttctgtcat agttagatgt |
| 3421 | gataattcta agagtgtcta tgacttattt ccttcactta attctatcca cagtcaaaaa |
| 3481 | tcccccaagg aggaaagctg aaagatgcac tgccatatta tctttcttaa ctttttccaa |
| 3541 | cacataatcc tctccaactg gattataaat aaattgaaaa taactcatta taccaattca |
| 3601 | ctattttatt ttttaatgaa ttaaaactag aaaacaaatt gatgcaaacc ctggaagtca |
| 3661 | gttgattact atatactaca gcagaatgac tcagatttca tagaaaggag caaccaaaat |
| 3721 | gtcacaaccc aaaactttac aagctttgct tcagaattag attgctttat aattcttgaa |
| 3781 | tgaggcaatt tcaagatatt tgtaaaagaa cagtaaacat tggtaagaat gagctttcaa |
| 3841 | ctcataggct tatttccaat ttaattgacc atactggata cttaggtcaa atttctgttc |
| 3901 | tctcttcccc aaataatatt aaagtattat ttgaactttt taagatgagg cagttcccct |
| 3961 | gaaaaagtta atgcagctct ccatcagaat ccactcttct agggatatga aaatctctta |
| 4021 | acacccaccc tacatacaca gacacacaca cacacacaca cacacacaca cacacacaca |
| 4081 | ttcaccctaa ggatccaatg gaatactgaa aagaaatcac ttccttgaaa attttattaa |
| 4141 | aaaacaaaca aacaaacaaa aagcctgtcc acccttgaga atccttcctc tccttggaac |
| 4201 | gtcaatgttt gtgtagatga aaccatctca tgctctgtgg ctccagggtt tctgttacta |
| 4261 | ttttatgcac ttgggagaag gcttagaata aaagatgtag cacattttgc tttcccattt |
| 4321 | attgtttggc cagctatgcc aatgtggtgc tattgtttct ttaagaaagt acttgactaa |
| 4381 | aaaaaaaaga aaaaaagaaa aaaaagaaag catagacata tttttttaaa gtataaaaac |
| 4441 | aacaattcta tagatagatg gcttaataaa atagcattag gtctatctag ccaccaccac |
| 4501 | ctttcaactt tttatcactc acaagtagtg tactgttcac caaattgtga atttgggggt |
| 4561 | gcaggggcag gagttggaaa ttttttaaag ttagaaggct ccattgtttt gttggctctc |
| 4621 | aaacttagca aaattagcaa tatattatcc aatcttctga acttgatcaa gagcatggag |
| 4681 | aataaacgcg ggaaaaaaga tcttataggc aaatagaaga atttaaaaga taagtaagtt |
| 4741 | ccttattgat ttttgtgcac tctgctctaa aacagatatt cagcaagtgg agaaaataag |
| 4801 | aacaaagaga aaaaatacat agatttacct gcaaaaaata gcttctgcca aatccccctt |
| 4861 | gggtattctt tggcatttac tggtttatag aagacattct cccttcaccc agacatctca |
| 4921 | aagagcagta gctctcatga aaagcaatca ctgatctcat ttgggaaatg ttggaaagta |
| 4981 | tttccttatg agatgggggt tatctactga taaagaaaga atttatgaga aattgttgaa |
| 5041 | agagatggct aacaatctgt gaagattttt tgtttcttgt ttttgttttt tttttttttt |
| 5101 | tactttatac agtctttatg aatttcttaa tgttcaaaat gacttggttc ttttcttctt |
| 5161 | tttttatatc agaatgagga ataataagtt aaacccacat agactcttta aaactatagg |
| 5221 | ctagatagaa atgtatgttt gacttgttga agctataatc agactattta aaatgttttg |
| 5281 | ctatttttaa tcttaaaaga ttgtgctaat ttattagagc agaacctgtt tggctctcct |
| 5341 | cagaagaaag aatctttcca ttcaaatcac atggctttcc accaatattt tcaaaagata |
| 5401 | aatctgattt atgcaatggc atcatttatt ttaaaacaga agaattgtga aagtttatgc |
| 5461 | ccctcccttg caaagaccat aaagtccaga tctggtaggg gggcaacaac aaaaggaaaa |
| 5521 | tgttgttgat tcttggtttt ggattttgtt ttgttttcaa tgctagtgtt taatcctgta |
| 5581 | gtacatattt gcttattgct attttaatat tttataagac cttcctgtta ggtattagaa |
| 5641 | agtgatacat agatatcttt tttgtgtaat ttctatttaa aaaagagaga agactgtcag |
| 5701 | aagctttaag tgcatatggt acaggataaa gatatcaatt taaataacca attcctatct |
| 5761 | ggaacaatgc ttttgttttt taaagaaacc tctcacagat aagacagagg cccaggggat |
| 5821 | ttttgaagct gtctttattc tgcccccatc ccaacccagc ccttattatt ttagtatctg |
| 5881 | cctcagaatt ttatagaggg ctgaccaagc tgaaactcta gaattaaagg aacctcactg |
| 5941 | aaaacatata tttcacgtgt tccctctttt tttttttcct ttttgtgaga tggggtctcg |
| 6001 | cactgtcccc caggctggag tgcagtggca tgatctcggc tcactgcaac ctccacctcc |
| 6061 | tgggtttaag cgattctcct gcctcagcct cctgagtagc tgggattaca ggcacccacc |
| 6121 | actatgcccg gctaattttt tggattttta atagagacgg ggttttacca tgttggccag |
| 6181 | gttggtctca aactcctgac cttgtgattt gcccgcctca gcctcccaaa ttgctgggat |
| 6241 | tacaggcatg agccaccaca ccctgcccat gtgttccctc ttaatgtatg attacatgga |
| 6301 | tcttaaacat gatccttctc tcctcattct tcaactatct ttgatggggt ctttcaaggg |
| 6361 | gaaaaaaatc caagcttttt taaagtaaaa aaaaaaaaag agaggacaca aaaccaaatg |
| 6421 | ttactgctca actgaaatat gagttaagat ggagacagag tttctcctaa taaccggagc |
| 6481 | tgaattacct ttcactttca aaaacatgac cttccacaat ccttagaatc tgcctttttt |
| 6541 | tatattactg aggcctaaaa gtaaacatta ctcattttat tttgcccaaa atgcactgat |
| 6601 | gtaaagtagg aaaaataaaa acagagctct aaaatccctt tcaagccacc cattgacccc |
| 6661 | actcaccaac tcatagcaaa gtcacttctg ttaatccctt aatctgattt tgtttggata |
| 6721 | tttatcttgt acccgctgct aaacacactg caggagggac tctgaaacct caagctgtct |
| 6781 | acttacatct tttatctgtg tctgtgtatc atgaaaatgt ctattcaaaa tatcaaaacc |
| 6841 | tttcaaatat cacgcagctt atattcagtt tacataaagg ccccaaatac catgtcagat |
| 6901 | ctttttggta aaagagttaa tgaactatga gaattgggat tacatcatgt attttgcctc |
| 6961 | atgtattttt atcacactta taggccaagt gtgataaata aacttacaga cactgaatta |
| 7021 | atttcccctg ctactttgaa accagaaaat aatgactggc cattcgttac atctgtctta |
| 7081 | gttgaaaagc atatttttta ttaaattaat tctgattgta tttgaaatta ttattcaatt |
| 7141 | cacttatggc agaggaatat caatcctaat gacttctaaa aatgtaacta attgaatcat |
| 7201 | tatcttacat ttactgttta ataagcatat tttgaaaatg tatggctaga gtgtcataat |
| 7261 | aaaatggtat atctttcttt agtaattaca ttaaaattag tcatgtttga ttaattagtt |
| 7321 | c |

The amino acid sequence of human IGF-1 isoform 4 preproprotein, provided by Genbank Accession No. NP_000609.1, is incorporated herein by reference, and is shown below (SEQ ID NO: 137).

| | |
|---|---|
| 1 | mgkisslptq lfkccfcdfl kvkmhtmsss hlfylalcll tftssatagp eticgaelvd |
| 61 | alqfvcgdrg fyfnkptgyg sssrrapqtg ivdeccfrsc dlrrlemyca plkpaksars |
| 121 | vraqrhtdmp ktqkevhlkn asrgsagnkn yrm |

The siRNA used to target human IGF-1 variant 4 mRNA include following sequences (SEQ ID NO: 138-141):

SEQ NO: 138:
5′-UAAACUGAAUAUAAGCUGC-3′
SEQ NO: 139:
5′-UAAAAAAAUAUGUCUAUGC-3′
SEQ NO: 140:
5′-UUUAACAGGUAACUCGUGC-3′
SEQ NO: 141:
5′-UAACAAACUACAAAAUAGC-3′

The molecular beacon used to target human IGF-1 variant 4 mRNA includes the following sequences (SEQ ID NO: 142-144):

SEQ NO 142:
5′-CCGGTC TAAACTGAATATAAGCTGCG GACCGG-3′
SEQ NO 143:
5′-CCGGTC TTTAAATTCTTCTATTTGCC GACCGG-3′
SEQ NO 144:
5′-CCGGTC TAATCAACTGACTTCCAGGGGACCGG-3′

The mRNA transcript sequence encoding human BMP-2, provided by Genbank Accession No. NM_001200.2, is incorporated herein by reference, and is shown below (SEQ ID NO: 145).

| | |
|---|---|
| 1 | ccacaaaggg cacttggccc cagggctagg agagcgaggg gagagcacag ccacccgcct |
| 61 | cggcggcccg ggactcggct cgactcgccg gagaatgcgc ccgaggacga cggggcgcca |
| 121 | gagccgcggt gctttcaact ggcgagcgcg aatgggggtg cactggagta aggcagagtg |
| 181 | atgcgggggg gcaactcgcc tggcaccgag atcgccgccg tgcccttccc tggacccggc |
| 241 | gtcgcccagg atggctgccc cgagccatgg gccgcggcgg agctagcgcg gagcgcccga |
| 301 | ccctcgaccc ccgagtcccg gagccggccc cgcgcggggc cacgcgtccc tcgggcgctg |
| 361 | gttcctaagg aggacgacag caccagcttc tcctttctcc cttcccttcc ctgccccgca |
| 421 | ctcctccccc tgctcgctgt tgttgtgtgt cagcacttgg ctggggactt cttgaacttg |
| 481 | cagggagaat aacttgcgca ccccactttg cgccggtgcc tttgccccag cggagcctgc |
| 541 | ttcgccatct ccgagcccca ccgcccctcc actcctcggc cttgcccgac actgagacgc |
| 601 | tgttcccagc gtgaaaagag agactgcgcg gccggcaccc gggagaagga ggaggcaaag |
| 661 | aaaaggaacg gacattcggt ccttgcgcca ggtcctttga ccagagtttt tccatgtgga |
| 721 | cgctctttca atggacgtgt ccccgcgtgc ttcttagacg gactgcggtc tcctaaaggt |
| 781 | cgaccatggt ggccgggacc cgctgtcttc tagcgttgct gcttccccag gtcctcctgg |
| 841 | gcggcgcggc tggcctcgtt ccggagctgg gccgcaggaa gttcgcggcg gcgtcgtcgg |
| 901 | gccgcccctc atcccagccc tctgacgagg tcctgagcga gttcgagttg cggctgctca |
| 961 | gcatgttcgg cctgaaacag agacccaccc ccagcaggga cgccgtggtg cccccctaca |
| 1021 | tgctagacct gtatcgcagg cactcaggtc agccgggctc acccgcccca gaccaccggt |
| 1081 | tggagagggc agccagccga gccaacactg tgcgcagctt ccaccatgaa gaatctttgg |
| 1141 | aagaactacc agaaacgagt gggaaaacaa cccggagatt cttctttaat ttaagttcta |
| 1201 | tccccacgga ggagtttatc acctcagcag agcttcaggt tttccgagaa cagatgcaag |
| 1261 | atgctttagg aaacaatagc agtttccatc accgaattaa tatttatgaa atcataaaac |
| 1321 | ctgcaacagc caactcgaaa ttccccgtga ccagactttt ggacaccagg ttggtgaatc |
| 1381 | agaatgcaag caggtgggaa agttttgatg tcacccccgc tgtgatgcgg tggactgcac |
| 1441 | agggacacgc caaccatgga ttcgtggtgg aagtggccca cttggaggag aaacaaggtg |
| 1501 | tctccaagag acatgttagg ataagcaggt ctttgcacca agatgaacac agctggtcac |
| 1561 | agataaggcc attgctagta acttttggcc atgatggaaa agggcatcct ctccacaaaa |
| 1621 | gagaaaaacg tcaagccaaa cacaaacagc ggaaacgcct taagtccagc tgtaagagac |
| 1681 | accctttgta cgtggacttc agtgacgtgg ggtggaatga ctggattgtg gctcccccgg |
| 1741 | ggtatcacgc cttttactgc cacggagaat gcccttttcc tctggctgat catctgaact |
| 1801 | ccactaatca tgccattgtt cagacgttgg tcaactctgt taactctaag attcctaagg |
| 1861 | catgctgtgt cccgacagaa ctcagtgcta tctcgatgct gtaccttgac gagaatgaaa |
| 1921 | aggttgtatt aaagaactat caggacatgg ttgtggaggg ttgtgggtgt cgctagtaca |
| 1981 | gcaaaattaa atacataaat atatatatat atatatattt tagaaaaaag aaaaaaacaa |
| 2041 | acaaacaaaa aaaccccacc ccagttgaca ctttaatatt tcccaatgaa gactttattt |
| 2101 | atggaatgga atggaaaaaa aaacagctat tttgaaaata tatttatatc tacgaaaaga |
| 2161 | agttgggaaa acaaatattt taatcagaga attattcctt aaagatttaa aatgtattta |
| 2221 | gttgtacatt ttatatgggt tcaaccccag cacatgaagt ataatggtca gatttatttt |
| 2281 | gtatttattt actattataa ccacttttta ggaaaaaaat agctaatttg tatttatatg |
| 2341 | taatcaaaag aagtatcggg tttgtacata attttccaaa aattgtagtt gttttcagtt |
| 2401 | gtgtgtattt aagatgaaaa gtctacatgg aaggttactc tggcaaagtg cttagcacgt |
| 2461 | ttgctttttt gcagtgctac tgttgagttc acaagttcaa gtccagaaaa aaaaagtgga |
| 2521 | taatccactc tgctgacttt caagattatt atattattca attctcagga atgttgcaga |
| 2581 | gtgattgtcc aatccatgag aatttacatc cttattaggt ggaatatttg gataagaacc |
| 2641 | agacattgct gatctattat agaaactctc ctcctgcccc ttaatttaca gaaagaataa |
| 2701 | agcaggatcc atagaaataa ttaggaaaac gatgaacctg caggaaagtg aatgatggtt |
| 2761 | tgttgttctt ctttcctaaa ttagtgatcc cttcaaaggg gctgatctgg ccaaagtatt |
| 2821 | caataaaacg taagatttct tcattattga tattgtggtc atatatattt aaaattgata |
| 2881 | tctcgtggcc ctcatcaagg gttggaaatt tatttgtgtt ttacctttac ctcatctgag |
| 2941 | agctctttat tctccaaaga acccagtttt ctaacttttt gcccaacacg cagcaaaatt |
| 3001 | atgcacatcg tgttttctgc ccaccctctg ttctctgacc tatcagcttg cttttctttc |
| 3061 | caaggttgtg tgtttgaaca catttctcca aatgttaaac ctatttcaga taataaatat |
| 3121 | caaatctctg gcatttcatt ctataaagtc |

The amino acid sequence of human BMP-2 preproprotein, provided by Genbank Accession No. NP_001191.1, is incorporated herein by reference, and is shown below (SEQ ID NO: 146).

| | |
|---|---|
| 1 | mvagtrclla lllpqvllgg aaglypelgr rkfaaassgr pssqpsdevl sefelrllsm |
| 61 | fglkqrptps rdavvppyml dlyrrhsgqp gspapdhrle raasrantvr sfhheeslee |
| 121 | lpetsgkttr rfffnlssip teefitsael qvfreqmqda lgnnssfhhr iniyeiikpa |
| 181 | tanskfpvtr lldtrlvnqn asrwesfdvt pavmrwtaqg hanhgfvvev ahleekqgvs |
| 241 | krhvrisrsl hqdehswsqi rpllvtfghd gkghplhkre krqakhkqrk rlkssckrhp |
| 301 | lyvdfsdvgw ndwivappgy hafychgecp fpladhlnst nhaivqtlvn svnskipkac |
| 361 | cvptelsais mlyldenekv vlknyqdmvv egcgcr (Signal protein AA 1-23; |
| | proprotein AA 24-396; mature protein AA 283-396). |

The siRNA used to target human BMP-2 mRNA include following sequences (SEQ ID NO: 147-150):

SEQ NO: 147:
5′-UUGUGAACUCAACAGUAGC-3′
SEQ NO: 148:
5′-UUAAUUUUGCUGUACUAGC-3′
SEQ NO: 149:
5′-UAAAACACAAAUAAAUUUC-3′
SEQ NO: 150:
5′-UUCUUUCUGUAAAUUAAGG-3′

The molecular beacon used to target human BMP-2 mRNA includes the following sequences (SEQ ID NO: 151-153):

SEQ NO 151:
5′-CCGGTC TAATACAAAATAAATCTG GACCGG-3′
SEQ NO 152:
5′-CCGGTC AAAACACAAATAAATTTCC GACCGG-3′
SEQ NO 153:
5′-CCGGTC TTCATTCTCGTCAAGGTAC GACCGG-3′

The mRNA transcript sequence encoding human BMP-4 variant 1, provided by Genbank Accession No. NM_001202.3, is incorporated herein by reference, and is shown below (SEQ ID NO: 154).

| | |
|---|---|
| 1 | aagaggagga aggaagatgc gagaaggcag aggaggaggg agggagggaa ggagcgcgga |
| 61 | gcccggcccg gaagctaggt gagtgtggca tccgagctga gggacgcgag cctgagacgc |
| 121 | cgctgctgct ccggctgagt atctagcttg tctccccgat gggattcccg tccaagctat |
| 181 | ctcgagcctg cagcgccaca gtccccggcc ctcgcccagg ttcactgcaa ccgttcagag |
| 241 | gtccccagga gctgctgctg gcgagcccgc tactgcaggg acctatggag ccattccgta |
| 301 | gtgccatccc gagcaacgca ctgctgcagc ttccctgagc ctttccagca agtttgttca |
| 361 | agattggctg tcaagaatca tggactgtta ttatatgcct tgttttctgt caagacacca |
| 421 | tgattcctgg taaccgaatg ctgatggtcg ttttattatg ccaagtcctg ctaggaggcg |
| 481 | cgagccatgc tagtttgata cctgagacgg ggaagaaaaa agtcgccgag attcagggcc |
| 541 | acgcgggagg acgccgctca gggcagagcc atgagctcct gcgggacttc gaggcgacac |
| 601 | ttctgcagat gtttgggctg cgccgccgcc cgcagcctag caagagtgcc gtcattccgg |
| 661 | actacatgcg ggatctttac cggcttcagt ctggggagga ggaggaagag cagatccaca |
| 721 | gcactggtct tgagtatcct gagcgcccgg ccagccgggc caacaccgtg aggagcttcc |
| 781 | accacgaaga acatctggag aacatcccag ggaccagtga aaactctgct tttcgtttcc |
| 841 | tctttaacct cagcagcatc cctgagaacg aggtgatctc ctctgcagag cttcggctct |
| 901 | tccgggagca ggtggaccag ggccctgatt gggaaagggg cttccaccgt ataaacattt |
| 961 | atgaggttat gaagccccca gcagaagtgg tgcctgggca cctcatcaca cgactactgg |
| 1021 | acacgagact ggtccaccac aatgtgacac ggtgggaaac ttttgatgtg agccctgcgg |
| 1081 | tccttcgctg gacccgggag aagcagccaa actatgggct agccattgag gtgactcacc |
| 1141 | tccatcagac tcggacccac cagggccagc atgtcaggat tagccgatcg ttacctcaag |
| 1201 | ggagtgggaa ttgggcccag ctccggcccc tcctggtcac ctttggccat gatggccggg |
| 1261 | gccatgcctt gacccgacgc cggagggcca agcgtagccc taagcatcac tcacagcggg |
| 1321 | ccaggaagaa gaataagaac tgccggcgcc actcgctcta tgtggacttc agcgatgtgg |
| 1381 | gctggaatga ctggattgtg gccccaccag gctaccaggc cttctactgc catggggact |
| 1441 | gcccctttcc actggctgac cacctcaact caaccaacca tgccattgtg cagaccctgg |
| 1501 | tcaattctgt caattccagt atccccaaag cctgttgtgt gcccactgaa ctgagtgcca |
| 1561 | tctccatgct gtacctggat gagtatgata aggtggtact gaaaaattat caggagatgg |
| 1621 | tagtagaggg atgtgggtgc cgctgagatc aggcagtcct tgaggataga cagatataca |
| 1681 | caccacacac acacaccaca tacaccacac acacacgttc ccatccactc acccacacac |
| 1741 | tacacagact gcttccttat agctggactt ttatttaaaa aaaaaaaaaa aaaaggaaaa |
| 1801 | aatccctaaa cattcacctt gaccttattt atgactttac gtgcaaatgt tttgaccata |
| 1861 | ttgatcatat attttgacaa aatatattta taactacgta ttaaaagaaa aaaataaaat |
| 1921 | gagtcattat tttaaaggta aaaaaaaaaa aaaaaaa |

The amino acid sequence of human BMP-4preproprotein, provided by Genbank Accession No. NP_001193.2, is incorporated herein by reference, and is shown below (SEQ ID NO: 155).

| | |
|---|---|
| 1 | mipgnrmlmv vllcqvllgg ashaslipet gkkkvaeiqg haggrrsgqs hellrdfeat |
| 61 | llqmfglrrr pqpsksavip dymrdlyrlq sgeeeeeqih stgleyperp asrantvrsf |
| 121 | hheehlenip gtsensafrf lfnlssipen evissaelrl freqvdqgpd wergfhrini |
| 181 | yevmkppaev vpghlitrll dtrlvhhnvt rwetfdvspa vlrwtrekqp nyglaievth |
| 241 | lhqtrthqgq hvrisrslpq gsgnwaqlrp llvtfghdgr ghaltrrrra krspkhhsqr |
| 301 | arkknkncrr hslyvdfsdv gwndwivapp gyqafychgd cpfpladhln stnhaivqtl |
| 361 | vnsvnssipk accvptelsa ismlyldeyd kvvlknyqem vvegcgcr (Signal peptide AA 1-24) |

The siRNA used to target human BMP-4 variant 1 mRNA include following sequences (SEQ ID NO: 156-159):

SEQ NO: 156:
5′-UAAUAAAACGACCAUCAGCA-3′
SEQ NO: 157:
5′-UAUCUGUCUAUCCUCAAGGA-3′
SEQ NO: 158:
5′-UUCUUAUUCUUCUUCCUGGC-3′
SEQ NO: 159:
5′-UAAUAAAACGACCAUCAGC-3′

The molecular beacon used to target human BMP-4 variant 1 mRNA includes the following sequences (SEQ ID NO: 160-162):

SEQ NO 160:
5′-CCGGTC TATCTGTCTATCCTCAAGG GACCGG-3′
SEQ NO 161:
5′-CCGGTC TCTCAGGTATCAAACTAGC GACCGG-3′
SEQ NO 162:
5′-CCGGTC TTTGTCAAAATATATGATC GACCGG-3′

The mRNA transcript sequence encoding human BMP-7, provided by Genbank Accession No. NM_001719.2, is incorporated herein by reference, and is shown below (SEQ ID NO: 163).

| | |
|---|---|
| 1 | agcgcgtacc actctggcgc tcccgaggcg gcctcttgtg cgatccaggg cgcacaaggc |
| 61 | tgggagagcg ccccggggcc cctgctatcc gcgccggagg ttggaagagg gtgggttgcc |
| 121 | gccgcccgag ggcgagagcg ccagaggagc gggaagaagg agcgctcgcc cgcccgcctg |
| 181 | cctcctcgct gcctccccgg cgttggctct ctggactcct aggcttgctg gctgctcctc |
| 241 | ccacccgcgc ccgcctcctc actcgccttt tcgttcgccg gggctgcttt ccaagccctg |
| 301 | cggtgcgccc gggcgagtgc ggggcgaggg gcccggggcc agcaccgagc agggggcggg |
| 361 | ggtccgggca gagcgcggcc ggccggggag gggccatgtc tggcgcgggc gcagcggggc |
| 421 | ccgtctgcag caagtgaccg agcggcgcgg acggccgcct gccccctctg ccacctgggg |
| 481 | cggtgcgggc ccggagcccg gagcccgggt agcgcgtaga gccggcgcga tgcacgtgcg |
| 541 | ctcactgcga gctgcggcgc cgcacagctt cgtggcgctc tgggcacccc tgttcctgct |
| 601 | gcgctccgcc ctggccgact tcagcctgga caacgaggtg cactcgagct tcatccaccg |
| 661 | gcgcctccgc agccaggagc ggcgggagat gcagcgcgag atcctctcca ttttgggctt |
| 721 | gccccaccgc ccgcgcccgc acctccaggg caagcacaac tcggcaccca tgttcatgct |
| 781 | ggacctgtac aacgccatgg cggtggagga gggcggcggg cccggcggcc agggcttctc |
| 841 | ctacccctac aaggccgtct tcagtaccca gggcccccct ctggccagcc tgcaagatag |
| 901 | ccatttcctc accgacgccg acatggtcat gagcttcgtc aacctcgtgg aacatgacaa |
| 961 | ggaattcttc cacccacgct accaccatcg agagttccgg tttgatcttt ccaagatccc |
| 1021 | agaaggggaa gctgtcacgg cagccgaatt ccggatctac aaggactaca tccgggaacg |
| 1081 | cttcgacaat gagacgttcc ggatcagcgt ttatcaggtg ctccaggagc acttgggcag |
| 1141 | ggaatcggat ctcttcctgc tcgacagccg taccctctgg gcctcggagg agggctggct |
| 1201 | ggtgtttgac atcacagcca ccagcaacca ctgggtggtc aatccgcggc acaacctggg |
| 1261 | cctgcagctc tcggtggaga cgctggatgg gcagagcatc aaccccaagt tggcgggcct |
| 1321 | gattgggcgg cacgggcccc agaacaagca gcccttcatg gtggctttct tcaaggccac |
| 1381 | ggaggtccac ttccgcagca tccggtccac ggggagcaaa cagcgcagcc agaaccgctc |
| 1441 | caagacgccc aagaaccagg aagccctgcg gatggccaac gtggcagaga acagcagcag |
| 1501 | cgaccagagg caggcctgta agaagcacga gctgtatgtc agcttccgag acctgggctg |
| 1561 | gcaggactgg atcatcgcgc ctgaaggcta cgccgcctac tactgtgagg gggagtgtgc |
| 1621 | cttccctctg aactcctaca tgaacgccac caaccacgcc atcgtgcaga cgctggtcca |
| 1681 | cttcatcaac ccggaaacgg tgcccaagcc ctgctgtgcg cccacgcagc tcaatgccat |
| 1741 | ctccgtcctc tacttcgatg acagctccaa cgtcatcctg aagaaataca gaaacatggt |
| 1801 | ggtccgggcc tgtggctgcc actagctcct ccgagaattc agaccctttg gggccaagtt |
| 1861 | tttctggatc ctccattgct cgccttggcc aggaaccagc agaccaactg ccttttgtga |
| 1921 | gaccttcccc tccctatccc caactttaaa ggtgtgagag tattaggaaa catgagcagc |
| 1981 | atatggcttt tgatcagttt ttcagtggca gcatccaatg aacaagatcc tacaagctgt |
| 2041 | gcaggcaaaa cctagcagga aaaaaaaaca acgcataaag aaaaatggcc gggccaggtc |
| 2101 | attggctggg aagtctcagc catgcacgga ctcgtttcca gaggtaatta tgagcgccta |
| 2161 | ccagccaggc cacccagccg tgggaggaag ggggcgtggc aaggggtggg cacattggtg |
| 2221 | tctgtgcgaa aggaaaattg acccggaagt tcctgtaata aatgtcacaa taaaacgaat |
| 2281 | gaatgaaaat ggttaggacg ttacagatat attttcctaa acaatttatc cccatttctc |
| 2341 | ggtttatcct gatgcgtaaa cagaagctgt gtcaagtgga gggcggggag gtccctctcc |
| 2401 | attccctaca gttttcatcc tgaggcttgc agaggcccag tgtttaccga ggtttgccca |
| 2461 | aatccaagat ctagtgggag gggaaagagc aaatgtctgc tccgaggagg gcggtgtgtt |
| 2521 | gatctttgga ggaaaaatat gttctgttgt tcagctggat ttgccgtggc agaaatgaaa |
| 2581 | ctaggtgtgt gaaatacccg cagacatttg ggattggctt ttcacctcgc cccagtggta |
| 2641 | gtaaatccat gtgaaattgc agaggggaca aggacagcaa gtaggatgga acttgcaact |
| 2701 | caaccctgtt gttaagaagc accaatgggc cgggcacagt agctcccacc tgtaatccca |
| 2761 | gcactttggg aggctgaggt gggcggatca tttgaggtca ggagttcgag accagcctgg |
| 2821 | ccaacatggt gaaaccccat ctctactaaa aatacaaaaa ttagccgggc atggtggcac |
| 2881 | gcacctgtaa tcccagctac tctggaggct gaggcaggag aattgcttga accccagagg |
| 2941 | tggaggttgc agtgagccaa gatcgtccca ctgcactcca gcttgggtga caaaacaaga |
| 3001 | ctccatctca aaagaaaaaa aaaacagcac caatgaagcc tagttctcca cgggagtggg |
| 3061 | gtgagcagga gcactgcaca tcgccccagt ggaccctctg gtctttgtct gcagtggcat |
| 3121 | tccaaggctg ggccctggca agggcacccg tggctgtctc ttcatttgca gaccctgatc |
| 3181 | agaagtctct gcaaacaaat ttgctccttg aattaagggg gagatggcat aataggaggt |
| 3241 | ctgatgggtg caggatgtgc tggacttaca ttgcaaatag aagccttgtt gagggtgaca |
| 3301 | tcctaaccaa gtgtcccgat ttggaggtgg catttctgac gtggctcttg gtgtaagcct |
| 3361 | gccttgcctt ggctggtgag tcccataaat agtatgcact cagcctccgg ccacaaacac |
| 3421 | aaggcctagg ggagggctag actgtctgca aacgttttct gcatctgtaa agaaaacaag |
| 3481 | gtgatcgaaa actgtggcca tgtggaaccc ggtcttgtgg gggactgttt ctccatcttg |
| 3541 | actcagacag ttcctggaaa caccggggct ctgtttttat tttctttgat gtttttcttc |
| 3601 | tttagtagct tgggctgcag cctccactct ctagtcactg gggaggagta ttttttgtta |
| 3661 | tgtttggttt catttgctgg cagagctggg gctttttgtg tgatccctct tggtgtgagt |
| 3721 | tttctgaccc aaccagcctc tggttagcat catttgtaca tttaaacctg taaatagttg |
| 3781 | ttacaaagca aagagattat ttatttccat ccaaagctct tttgaacacc cccccccctt |
| 3841 | taatccctcg ttcaggacga tgagcttgct ttccttcaac ctgtttgttt tcttatttaa |
| 3901 | gactatttat taatggttgg accaatgtac tcacagctgt tgcgtcgagc agtccttagt |
| 3961 | gaaaattctg tataaataga caaaatgaaa agggtttgac cttgcaataa aaggagacgt |
| 4021 | ttggttctgg caaaaaaaaa aaaaaaaaa |

The amino acid sequence of human BMP-7 precursor, provided by Genbank Accession No. NP_001710.1, is incorporated herein by reference, and is shown below (SEQ ID NO: 164).

```
    1 mhvrslraaa phsfvalwap lfllrsalad fsldnevhss fihrrlrsqe rremqreils
  61 ilglphrprp hlqgkhnsap mfmldlynam aveegggpgg qgfsypykav fstqgpplas
121 lqdshfltda dmvmsfvnlv ehdkeffhpr yhhrefrfdl skipegeavt aaefriykdy
181 irerfdnetf risvyqvlqe hlgresdlfl ldsrtlwase egwlvfdita tsnhwvvnpr
241 hnlglqlsve tldgqsinpk lagligrhgp qnkqpfmvaf fkatevhfrs irstgskqrs
301 qnrsktpknq ealrmanvae nsssdqrqac kkhelyvsfr dlgwqdwiia pegyaayyce
361 gecafplnsy mnatnhaivq tlvhfinpet vpkpccaptq lnaisvlyfd dssnvilkky
421 rnmvvracgc h (signal peptide AA 1-29; mature peptide AA 293-431).
```

The siRNA used to target human BMP-7 mRNA include following sequences (SEQ ID NO: 165-168):

SEQ NO: 165:
5′-UUCCUAAUACUCUCACACC-3′
SEQ NO: 166:
5′-UAACAAAAAAUACUCCUCC-3′
SEQ NO: 167:
5′-UAAAUAAGAAAACAAACAGG-3′
SEQ NO: 168:
5′-UUCCUAAUACUCUCACACCU-3′

The molecular beacon used to target human BMP-7 mRNA includes the following sequences (SEQ ID NO: 169-171):

SEQ NO 169:
5′-CCGGTC TAACAAAAAATACTCCTCCC GACCGG-3′
SEQ NO 170:
5′-CCGGTC TTGTAACAACUATTTACAGG GACCGG-3′
SEQ NO 171:
5′-CCGGTC TAAATAAGAAAACAAACAG GACCGG-3′

The mRNA transcript sequence encoding human IL-1 receptor antagonist variant 3, provided by Genbank Accession No. NM_000577.4, is incorporated herein by reference, and is shown below (SEQ ID NO: 172).

```
      1 gggcagctcc accctgggag ggactgtggc ccaggtactg cccgggtgct actttatggg
    61 cagcagctca gttgagttag agtctggaag acctcagaag acctcctgtc ctatgaggcc
  121 ctccccatgg ctttagagac gatctgccga ccctctggga gaaaatccag caagatgcaa
  181 gccttcagaa tctgggatgt taaccagaag accttctatc tgaggaacaa ccaactagtt
  241 gctggatact tgcaaggacc aaatgtcaat ttagaagaaa agatagatgt ggtacccatt
  301 gagcctcatg ctctgttctt gggaatccat ggagggaaga tgtgcctgtc ctgtgtcaag
  361 tctggtgatg agaccagact ccagctggag gcagttaaca tcactgacct gagcgagaac
  421 agaaagcagg acaagcgctt cgccttcatc cgctcagaca gtggccccac caccagtttt
  481 gagtctgccg cctgccccgg ttggttcctc tgcacagcga tggaagctga ccagcccgtc
  541 agcctcacca atatgcctga cgaaggcgtc atggtcacca aattctactt ccaggaggac
  601 gagtagtact gcccaggcct gcctgttccc attcttgcat ggcaaggact gcagggactg
  661 ccagtccccc tgccccaggg ctcccggcta tgggggcact gaggaccagc cattgagggg
  721 tggaccctca gaaggcgtca caacaacctg gtcacaggac tctgcctcct cttcaactga
  781 ccagcctcca tgctgcctcc agaatggtct ttctaatgtg tgaatcagag cacagcagcc
  841 cctgcacaaa gcccttccat gtcgcctctg cattcaggat caaaccccga ccacctgccc
  901 aacctgctct cctcttgcca ctgcctcttc ctccctcatt ccaccttccc atgccctgga
  961 tccatcaggc cacttgatga cccccaacca agtggctccc acaccctgtt ttacaaaaaa
1021 gaaaagacca gtccatgagg gaggttttta agggtttgtg gaaaatgaaa attaggattt
1081 catgattttt ttttttcagt ccccgtgaag gagagccctt catttggaga ttatgttctt
1141 tcggggagag gctgaggact taaaatattc ctgcatttgt gaaatgatgg tgaaagtaag
1201 tggtagcttt tcccttcttt ttcttctttt tttgtgatgt cccaacttgt aaaaattaaa
1261 agttatggta ctatgttagc cccataattt tttttttcct tttaaaacac ttccataatc
1321 tggactcctc tgtccaggca ctgctgccca gcctccaagc tccatctcca ctccagattt
1381 tttacagctg cctgcagtac tttacctcct atcagaagtt tctcagctcc caaggctctg
1441 agcaaatgtg gctcctgggg gttctttctt cctctgctga aggaataaat tgctccttga
1501 cattgtagag cttctggcac ttggagactt gtatgaaaga tggctgtgcc tctgcctgtc
1561 tcccccaccg ggctgggagc tctgcagagc aggaaacatg actcgtatat gtctcaggtc
1621 cctgcagggc caagcaccta gcctcgctct tggcaggtac tcagcgaatg aatgctgtat
1681 atgttgggtg caaagttccc tacttcctgt gacttcagct ctgttttaca ataaaatctt
1741 gaaaatgcct aaaaaaaaaa aaaaaaaaaa aaaaaaaaaa aaaaaaaaaa aaaaaaaaaa
1801 aa
```

The amino acid sequence of human IL-1 receptor antagonist isoform 3, provided by Genbank Accession No. NP_000568.1, is incorporated herein by reference, and is shown below (SEQ ID NO: 173).

```
    1 maleticrps grksskmqaf riwdvnqktf ylrnnqlvag ylqgpnvnle ekidvvpiep
  61 halflgihgg kmclscvksg detrlqleav nitdlsenrk qdkrfafirs dsgpttsfes
121 aacpgwflct ameadqpvsl tnmpdegvmv tkfyfqede
```

The Pre-miRNA sequence of human microRNA140, provided by Genbank Accession NO: NR_029681.1, is incorporated herein by reference, and is shown below (SEQ ID NO: 174).

5′-UGUGUCUCUCUCUGUGUCCUGCCAGUGGUUUUACCCUAUGGUAGGUU
ACGUCAUGCUGUUCUACCACAGGGUAGAACCACGGACAGGAUACCGGGGC
ACC-3′

And mature microRNA140 (SEQ ID NO: 175).

5′-cagugguuuuacccuaugguag-3′

The Pre-miRNA sequence of human microRNA365, provided by Genbank Accession NO: NR_029854.1, is incorporated herein by reference, and is shown below (SEQ ID NO: 176).

5′-ACCGCAGGGAAAAUGAGGGACUUUUGGGGGCAGAUGUGUUUCCAUUC
CACUAUCAUAAUGCCCCUAAAAAUCCUUAUUGCUCUUGCA-3′

And mature microRNA365(SEQ ID NO: 177):

5′-AGGGACUUUUGGGGGCAGAUGUG-3′

The Pre-miRNA sequence of human microRNA125a, provided by Genbank Accession NO: NR_029693.1, is incorporated herein by reference, and is shown below (SEQ ID NO: 178).

5′-UGCCAGUCUCUAGGUCCCUGAGACCCUUUAACCUGUGAGGACAUCCA
GGGUCACAGGUGAGGUUCUUGGGAGCCUGGCGUCUGGCC-3′

And two mature microRNA125a (SEQ ID NO: 179-180):

SEQ ID NO: 179:
hsa-mir-125a-5p: 5′-ucccugagacccuuuaaccuguga-3′\
SEQ ID NO: 180:
hsa-mir-125a-3p: 5′-acaggugagguucuugggagcc-3′

The mRNA sequence encoding human IL-15, provided by Genbank Accession No. BC018149.2, is incorporated herein by reference, and is shown below (SEQ ID NO: 181).

```
      1 actccgggtg gcaggcgccc gggggaatcc cagctgactc gctcactgcc ttcgaagtcc
    61 ggcgcccccc gggagggaac tgggtggccg caccctcccg gctgcggtgg ctgtcgcccc
  121 ccaccctgca gccaggactc gatggaggta cagagctcgg cttctttgcc ttgggagggg
  181 agtggtggtg gttgaaaggg cgatggaatt ttccccgaaa gcctacgccc agggcccctc
  241 ccagctccag cgttaccctc cggtctatcc tactggccga gctgccccgc cttctcatgg
  301 ggaaaactta gccgcaactt caatttttgg tttttccttt aatgacactt ctgaggctct
  361 cctagccatc ctcccgcttc cggaggagcg cagatcgcag gtccctttgc ccctggcgtg
  421 cgactcccta ctgcgctgcg ctcttacggc gttccaggct gctggctagc gcaaggcggg
  481 ccgggcaccc cgcgctccgc tgggagggtg agggacgcgc gtctggcggc cccagccaag
  541 ctgcgggttt ctgagaagac gctgtcccgc agccctgagg gctgagttct gcacccagtc
  601 aagctcagga aggccaagaa aagaatccat tccaatatat ggccatgtgg ctctttggag
  661 caatgttcca tcatgttcca tgctgctgac gtcacatgga gcacagaaat caatgttagc
  721 agatagccag cccatacaag atcgtattgt attgtaggag gcatcgtgga tggatggctg
  781 ctggaaaccc cttgccatag ccagctcttc ttcaatactt aaggatttac cgtggctttg
  841 agtaatgaga atttcgaaac cacatttgag aagtatttcc atccagtgct acttgtgttt
  901 acttctaaac agtcattttc taactgaagc tggcattcat gtcttcattt tgggctgttt
  961 cagtgcaggg cttcctaaaa cagaagccaa ctgggtgaat gtaataagtg atttgaaaaa
1021 aattgaagat cttattcaat ctatgcatat tgatgctact ttatatacgg aaagtgatgt
1081 tcaccccagt tgcaaagtaa cagcaatgaa gtgctttctc ttggagttac aagttatttc
1141 acttgagtcc ggagatgcaa gtattcatga tacagtagaa aatctgatca tcctagcaaa
1201 caacagtttg tcttctaatg ggaatgtaac agaatctgga tgcaaagaat gtgaggaact
1261 ggaggaaaaa aatattaaag aatttttgca gagttttgta catattgtcc aaatgttcat
1321 caacacttct tgattgcaat tgattctttt taaagtgttt ctgttattaa caaacatcac
1381 tctgctgctt agacataaca aaacactcgg catttcaaat gtgctgtcaa aacaagtttt
1441 tctgtcaaga agatgatcag accttggatc agatgaactc ttagaaatga aggcagaaaa
1501 atgtcattga gtaatatagt gactatgaac ttctctcaga cttactttac tcattttttt
1561 aatttattat tgaaattgta catatttgtg gaataatgta aaatgttgaa taaaaatatg
1621 tacaagtgtt gttttttaag ttgcactgat attttacctc ttattgcaaa atagcatttg
1681 tttaagggtg atagtcaaat tatgtattgg tggggctggg taccaatgct gcaggtcaac
1741 agctatgctg gtaggctcct gcctgtgtgg aaccactgac tactggctct cattgacttc
1801 cttactaagc atagcaaaca gaggaagaat ttgttatcag taagaaaaag aagaactata
1861 tgtgaatcct cttctttaca ctgtaattta gttattgatg tataaagcaa ctgttatgaa
1921 ataaagaaat tgcaataact ggcaaaaaaa aaaaaaaaaa aaaaaaaa
```

The atg start and stop codons are bolded and underlined. The amino acid sequence of human IL-15, provided by Genbank Accession No. AAH18149.1, is incorporated herein by reference, and is shown below (SEQ ID NO: 182).

```
    1 mriskphlrs isiqcylcll lnshflteag ihvfilgcfs aglpkteanw vnvisdlkki
  61 edliqsmhid atlytesdvh psckvtamkc fllelqvisl esgdasihdt venliilann
121 slssngnvte sgckeceele eknikeflqs fvhivqmfin ts (SEQ ID NO: 182)
```

The mRNA sequence encoding human IL-20 (interleukin-20 precursor), provided by Genbank Accession No. NM_018724.3, is incorporated herein by reference, and is shown below (SEQ ID NO: 183).

```
      1 ctttgaattc ctagctcctg tggtctccag atttcaggcc taagatgaaa gcctctagtc
    61 ttgccttcag ccttctctct gctgcgtttt atctcctatg gactccttcc actggactga
  121 agacactcaa tttgggaagc tgtgtgatcg ccacaaacct tcaggaaata cgaaatggat
  181 tttctgagat acggggcagt gtgcaagcca aagatggaaa cattgacatc agaatcttaa
  241 ggaggactga gtctttgcaa gacacaaagc ctgcgaatcg atgctgcctc ctgcgccatt
  301 tgctaagact ctatctggac agggtattta aaaactacca gacccctgac cattatactc
  361 tccggaagat cagcagcctc gccaattcct ttcttaccat caagaaggac ctccggctct
  421 gtcatgccca catgacatgc cattgtgggg aggaagcaat gaagaaatac agccagattc
  481 tgagtcactt tgaaaagctg gaacctcagg cagcagttgt gaaggctttg ggggaactag
  541 acattcttct gcaatggatg gaggagacag aataggagga aagtgatgct gctgctaaga
  601 atattcgagg tcaagagctc cagtcttcaa tacctgcaga ggaggcatga ccccaaacca
  661 ccatctcttt actgtactag tcttgtgctg gtcacagtgt atcttattta tgcattactt
  721 gcttccttgc atgattgtct ttatgcatcc ccaatcttaa ttgagaccat acttgtataa
  781 gatttttgta atatctttct gctattggat atatttatta gttaatatat ttatttattt
  841 tttgctattt aatgtattta tttttttact tggacatgaa actttaaaaa aattcacaga
  901 ttatatttat aacctgacta gagcaggtga tgtattttta tacagtaaaa aaaaaaaacc
  961 ttgtaaattc tagaagagtg gctagggggg ttattcattt gtattcaact aaggacatat
1021 ttactcatgc tgatgctctg tgagatattt gaaattgaac caatgactac ttaggatggg
1081 ttgtggaata agttttgatg tggaattgca catctacctt acaattactg accatcccca
1141 gtagactccc cagtcccata attgtgtatc ttccagccag gaatcctaca cggccagcat
1201 gtatttctac aaataaagtt ttctttgcat aacaaaaaaa aaaaaaaaaa aa
```

The atg start and stop codons are bolded and underlined. The amino acid sequence of human IL-20 (interleukin-20 precursor), provided by Genbank Accession No. NP_061194.2, is incorporated herein by reference, and is shown below (SEQ ID NO: 184).

```
    1 mkasslafsl lsaafyllwt pstglktlnl gscviatnlq eirngfseir gsvqakdgni
  61 dirilrrtes lqdtkpanrc cllrhllrly ldrvfknyqt pdhytlrkis slansfltik
121 kdlrlchahm tchcgeeamk kysqilshfe klepqaavvk algeldillq wmeete
```

The mRNA sequence encoding human PADI4 (protein-arginine deiminase type-4), provided by Genbank Accession No. NM_012387.2, is incorporated herein by reference, and is shown below (SEQ ID NO: 185).

| (SEQ ID NO: 185) | |
|---|---|
| 1 | acagccagag ggacgagcta gcccgacgat ggcccagggg acattgatcc gtgtgacccc |
| 61 | agagcagccc acccatgccg tgtgtgtgct gggcaccttg actcagcttg acatctgcag |
| 121 | ctctgcccct gaggactgca cgtccttcag catcaacgcc tccccagggg tggtcgtgga |
| 181 | tattgcccac ggccctccag ccaagaagaa atccacaggt tcctccacat ggcccctgga |
| 241 | ccctggggta gaggtgaccc tgacgatgaa agtggccagt ggtagcacag gcgaccagaa |
| 301 | ggttcagatt tcatactacg gacccaagac tccaccagtc aaagctctac tctacctcac |
| 361 | cggggtggaa atctccttgt gcgcagacat cacccgcacc ggcaaagtga agccaaccag |
| 421 | agctgtgaaa gatcagagga cctggacctg gggcccttgt ggacagggtg ccatcctgct |
| 481 | ggtgaactgt gacagagaca atctcgaatc ttctgccatg gactgcgagg atgatgaagt |
| 541 | gcttgacagc gaagacctgc aggacatgtc gctgatgacc ctgagcacga agacccccaa |
| 601 | ggacttcttc acaaaccata cactggtgct ccacgtggcc aggtctgaga tggacaaagt |
| 661 | gagggtgttt caggccacac ggggcaaact gtcctccaag tgcagcgtag tcttgggtcc |
| 721 | caagtggccc tctcactacc tgatggtccc cggtggaaag cacaacatgg acttctacgt |
| 781 | ggaggccctc gctttcccgg acaccgactt cccggggctc attaccctca ccatctccct |
| 841 | gctggacacg tccaacctgg agctccccga ggctgtggtg ttccaagaca gcgtggtctt |
| 901 | ccgcgtggcg ccctggatca tgacccccaa cacccagccc ccgcaggagg tgtacgcgtg |
| 961 | cagtattttt gaaaatgagg acttcctgaa gtcagtgact actctggcca tgaaagccaa |
| 1021 | gtgcaagctg accatctgcc ctgaggagga gaacatggat gaccagtgga tgcaggatga |
| 1081 | aatggagatc ggctacatcc aagccccaca caaaacgctg cccgtggtct tcgactctcc |
| 1141 | aaggaacaga ggcctgaagg agtttcccat caaacgcgtg atgggtccag attttggcta |
| 1201 | tgtaactcga gggccccaaa cagggggtat cagtggactg gactcctttg ggaacctgga |
| 1261 | agtgagcccc ccagtcacag tcaggggcaa ggaatacccg ctgggcagga ttctcttcgg |
| 1321 | ggacagctgt tatcccagca atgacagccg gcagatgcac caggccctgc aggacttcct |
| 1381 | cagtgcccag caggtgcagg cccctgtgaa gctctattct gactggctgt ccgtgggcca |
| 1441 | cgtggacgag ttcctgagct ttgtgccagc acccgacagg aagggcttcc ggctgctcct |
| 1501 | ggccagcccc aggtcctgct acaaactgtt ccaggagcag cagaatgagg gccacgggga |
| 1561 | ggccctgctg ttcgaaggga tcaagaaaaa aaaacagcag aaaataaaga acattctgtc |
| 1621 | aaacaagaca ttgagagaac ataattcatt tgtggagaga tgcatcgact ggaaccgcga |
| 1681 | gctgctgaag cgggagctgg gcctggccga gagtgacatc attgacatcc cgcagctctt |
| 1741 | caagctcaaa gagttctcta aggcggaagc ttttttcccc aacatggtga acatgctggt |
| 1801 | gctagggaag cacctgggca tccccaagcc cttcgggccc gtcatcaacg gccgctgctg |
| 1861 | cctggaggag aaggtgtgtt ccctgctgga gccactgggc ctccagtgca ccttcatcaa |
| 1921 | cgacttcttc acctaccaca tcaggcatgg ggaggtgcac tgcggcacca acgtgcgcag |
| 1981 | aaagcccttc tccttcaagt ggtggaacat ggtgccctga gcccatcttc cctggcgtcc |
| 2041 | tctccctcct ggccagatgt cgctgggtcc tctgcagtgt ggcaagcaag agctcttgtg |
| 2101 | aatattgtgg ctccctgggg gcggccagcc ctcccagcag tggcttgctt tcttctcctg |
| 2161 | tgatgtccca gtttcccact ctgaagatcc caacatggtc ctagcactgc acactcagtt |
| 2221 | ctgctctaag aagctgcaat aaagtttttt taagtcactt tgtac |

The atg start and stop codons are bolded and underlined. The amino acid sequence of human PADI4 (protein-arginine deiminase type-4) provided by Genbank Accession No. NP_036519.2, is incorporated herein by reference, and is shown below (SEQ ID NO: 186).

| (SEQ ID NO: 186) | |
|---|---|
| 1 | maqgtlirvt peqpthavcv lgtltqldic ssapedctsf sinaspgvvv diahgppakk |
| 61 | kstgsstwpl dpgvevtltm kvasgstgdq kvqisyygpk tppvkallyl tgveislcad |
| 121 | itrtgkvkpt ravkdqrtwt wgpcgqgail lvncdrdnle ssamdcedde vldsedlqdm |
| 181 | slmtlstktp kdfftnhtiv lhvarsemdk vrvfqatrgk lsskcsvvlg pkwpshylmv |
| 241 | pggkhnmdfy vealafpdtd fpglitltis lldtsnlelp eavvfqdsvv frvapwimtp |
| 301 | ntqppqevya csifenedfl ksvttlamka kcklticpee enmddqwmqd emeigyiqap |
| 361 | hktlpvvfds prnrglkefp ikrvmgpdfg yvtrgpqtgg isgldsfgnl evsppvtvrg |
| 421 | keyplgrilf gdscypsnds rqmhqalqdf lsaqqvqapv klysdwlsvg hvdeflsfvp |
| 481 | apdrkgfrll lasprscykl fqeqqneghg eallfegikk kkqqkiknil snktlrehns |
| 541 | fvercidwnr ellkrelgla esdiidipql fklkefskae affpnmvnml vlgkhlgipk |
| 601 | pfgpvingrc cleekvcsll eplglqctfi ndfftyhirh gevhcgtnvr rkpfsfkwwn |
| 661 | mvp |

The mRNA sequence encoding human HLA-DRB1, provided by Genbank Accession No. HQ267233.1, is incorporated herein by reference, and is shown below (SEQ ID NO: 187).

| | |
|---|---|
| 1 | atggtgtgtc tgaggctccc tggaggctcc tgcatggcag ttctgacagt gacactgatg |
| 61 | gtgctgagct ccccactggc tttggctggg gacaccagac cacgtttctt ggaggaggtt |
| 121 | aagtttgagt gtcatttctt caacgggacg gagcgggtgc ggttgctgga aagacgcgtc |
| 181 | cataaccaag aggagtacgc gcgctacgac agcgacgtgg gggagtaccg ggcggtgacg |
| 241 | gagctggggc ggcctgatgc cgagtactgg aacagccaga aggacctcct ggagcggagg |
| 301 | cgtgccgcgg tggacaccta ctgcagacac aactacgggg ttggtgagag cttcacagtg |
| 361 | cagcggcgag ttcaacctaa ggtgactgtg tatccttcaa agacccagcc cctgcagcac |
| 421 | cacaacctcc tggtctgttc tgtgaatggt ttctatccag gcagcattga agtcaggtgg |
| 481 | ttccggaacg gccaggaaga gaagactggg gtggtgtcca cgggcctgat ccagaatgga |
| 541 | gactggacct tccagaccct ggtgatgctg gaaacagttc ctcagagtgg agaggtttac |
| 601 | acctgccaag tggagcaccc aagtgtgatg agccctctca cagtggaatg gagagcacgg |
| 661 | tctgaatctg cacagagcaa gatgctgagt ggagtcgggg gctttgtgct gggcctgctc |
| 721 | ttccttgggg ccgggctgtt catctacttc aggaatcaga aaggacactc tggacttccg |
| 781 | ccaacaggat tcctgagctg a |

The atg start and stop codons are bolded and underlined. The amino acid sequence of human HLA-DRB1, provided by Genbank Accession No. ADZ73424.1, is incorporated herein by reference, and is shown below (SEQ ID NO: 188).

| (SEQ ID NO: 188) | |
|---|---|
| 1 | myclrlpggs cmavltvtlm vlssplalag dtrprfleev kfechffngt ervrllerry |
| 61 | hnqeeyaryd sdvgeyravt elgrpdaeyw nsqkdllerr raavdtycrh nygvgesftv |
| 121 | qrrvqpkvtv ypsktqplqh hnllvcsvng fypgsievrw frngqeektg vvstgliqng |
| 181 | dwtfqtivml etvpqsgevy tcqvehpsvm spltvewrar sesaqskmls gvggfvlgll |
| 241 | flgaglfiyf rnqkghsglp ptgfls |

The mRNA sequence encoding human PTPN22 provided by Genbank Accession No. BC071670.1, is incorporated herein by reference, and is shown below (SEQ ID NO: 189).

| | |
|---|---|
| 1 | ggtgtctcgg ccatgacaca catttgacat gccctccctc aacctactta tagactattt |
| 61 | ttcttgctct gcagcatgga ccaaagagaa attctgcaga agttcctgga tgaggcccaa |
| 121 | agcaagaaaa ttactaaaga ggagtttgcc aatgaatttc tgaagctgaa aaggcaatct |
| 181 | accaagtaca aggcagacaa aacctatcct acaactgtgg ctgagaagcc caagaatatc |
| 241 | aagaaaaaca gatataagga tattttgccc tatgattata gccgggtaga actatccctg |
| 301 | ataacctctg atgaggattc cagctacatc aatgccaact tcattaaggg agtttatgga |
| 361 | cccaaggctt atattgccac ccagggtcct ttatctacaa ccctcctgga cttctggagg |
| 421 | atgatttggg aatatagtgt ccttatcatt gttatggcat gcatggagta tgaaatggga |
| 481 | aagaaaaagt gtgagcgcta ctgggctgag ccaggagaga tgcagctgga atttggccct |
| 541 | ttctctgtat cctgtgaagc tgaaaaaagg aaatctgatt atataatcag gactctaaaa |
| 601 | gttaagttca atagtgaaac tcgaactatc taccagtttc attacaagaa ttggccagac |
| 661 | catgatgtac cttcatctat agaccctatt cttgagctca tctgggatgt acgttgttac |
| 721 | caagaggatg acagtgttcc catatgcatt cactgcagtg ctggctgtgg aaggactggt |
| 781 | gttatttgtg ctattgatta tacatggatg ttgctaaaag atgggagtca agcaaagcat |
| 841 | tgtattcctg agaaaaatca cactctccaa gcagactctt attctcctaa tttaccaaaa |
| 901 | agtaccacaa aagcagcaaa aatgatgaac caacaaagga caaaaatgga aatcaaagaa |
| 961 | tcttcttcct ttgactttag gacttctgaa ataagtgcaa aagaagagct agttttgcac |
| 1021 | cctgctaaat caagcacttc ttttgacttt ctggagctaa attacagttt tgacaaaaat |
| 1081 | gctgacacaa ccatgaaatg gcagacaaag gcatttccaa tagttgggga gcctcttcag |
| 1141 | aagcatcaaa gtttggattt gggctctctt ttgtttgagg gatgttctaa ttctaaacct |
| 1201 | gtaaatgcag caggaagata ttttaattca aaggtgccaa taacacggac caaatcaact |
| 1261 | ccttttgaat tgatacagca gagagaaacc aaggaggtgg acagcaagga aaacttttct |
| 1321 | tatttggaat ctcaaccaca tgattcttgt tttgtagaga tgcaggctca aaaagtaatg |
| 1381 | catgtttctt cagcagaact gaattattca ctgccatatg actctaaaca ccaaatacgt |
| 1441 | aatgcctcta atgtaaagca ccatgactct agtgctcttg gtgtatattc ttacatacct |
| 1501 | ttagtggaaa atccttattt ttcatcatgg cctccaagtg gtaccagttc taagatgtct |
| 1561 | cttgatttac ctgagaagca agatggaact gtttttcctt cttctctgtt gccaacatcc |
| 1621 | tctacatccc tcttctctta ttacaattca catgattctt tatcactgaa ttctccaacc |
| 1681 | aatatttcct cactattgaa ccaggagtca gctgtactag caactgctcc aaggatagat |
| 1741 | gatgaaatcc cccctccact tcctgtacgg acacctgaat catttattgt ggttgaggaa |
| 1801 | gctggagaat tctcaccaaa tgttcccaaa tccttatcct cagctgtgaa ggtaaaaatt |
| 1861 | ggaacatcac tggaatgggg tggaacatct gaaccaaaga aatttgatga ctctgtgata |
| 1921 | cttagaccaa gcaagagtgt aaaactccga agtcctaaat cagaactaca tcaagatcgt |
| 1981 | tcttctcccc cacctcctct cccagaaaga actctagagt ccttctttct tgccgatgaa |
| 2041 | gattgtatgc aggcccaatc tatagaaaca tattctacta gctatcctga caccatggaa |
| 2101 | aattcaacat cttcaaaaca gacactgaag actcctggaa aaagtttcac aaggagtaag |
| 2161 | agtttgaaaa ttttgcgaaa catgaaaaag agtatctgta attcttgccc accaaacaag |
| 2221 | cctgcagaat ctgttcagtc aaataactcc agctcatttc tgaattttgg ttttgcaaac |
| 2281 | cgtttttcaa aacccaaagg accaaggaat ccaccaccaa cttggaatat ttaataaaac |
| 2341 | tccagattta taataatatg ggctgcaagt acacctgcaa ataaaactac tagaatactg |
| 2401 | ctagttaaaa taagtgctct atatgcataa tatcaaatat gaagatatgc taatgtgtta |
| 2461 | atagctttta aaagaaaagc aaaatgccaa taagtgccag ttttgcattt tcatatcatt |
| 2521 | tgcattgagt tgaaaactgc aaataaaagt ttgtcacttg agcttatgta cagaatgcta |
| 2581 | tatgagaaac acttttagaa tggatttatt tttcattttt gccagttatt tttattttct |
| 2641 | tttacttttt tacataaaca taaacttcaa aaggtttgta agatttggat ctcaactaat |
| 2701 | ttctacattg ccagaatata ctataaaaag ttaaaaaaaa aacttacttt gtgggttgca |
| 2761 | atacaaactg ctcttgacaa tgactattcc ctgacagtta tttttgccta aatggagtat |
| 2821 | accttgtaaa tcttcccaaa tgttgtggaa aactggaata ttaagaaaat gagaaattat |
| 2881 | atttattaga ataaaatgtg caaataatga caattatttg aatgtaacaa ggaattcaac |
| 2941 | tgaaatcctg ataagtttta accaaagtca ttaaattacc aattctagaa aagtaatcaa |
| 3001 | tgaaatataa tagctatctt ttggtagcaa aagatataaa ttgtatatgt ttatacagga |
| 3061 | tctttcagat catgtgcaat ttttatctaa ccaatcagaa atactagttt aaaatgaatt |
| 3121 | tctatatgaa tatggatctg ccataagaaa atctagttca actctaattt tatgtagtaa |
| 3181 | ataaattggc aggtaattgt ttttacaaag aatccacctg acttccccta atgcattaaa |
| 3241 | aatattttta tttaaataac tttatttata acttttagaa acatgtagta ttgtttaaac |
| 3301 | atcatttgtt cttcagtatt tttcatttgg aagtccaata gggcaaattg aatgaagtat |
| 3361 | tattatctgt ctcttgtagt acaatgtatc caacagacac tcaataaact ttttggttgt |
| 3421 | taaaaaaaaa aaaaaa |

The atg start and stop codons are bolded and underlined. The amino acid sequence of human PTPN22, provided by Genbank Accession No. AAH716701.1, is incorporated herein by reference, and is shown below (SEQ ID NO: 190).

| (SEQ ID NO: 190) | |
|---|---|
| 1 | mdqreilqkf ldeaqskkit keefaneflk lkrqstkyka dktypttvae kpknikknry |
| 61 | kdilpydysr velslitsde dssyinanfi kgvygpkayi atqgplsttl ldfwrmiwey |
| 121 | svliivmacm eyemgkkkce rywaepgemq lefgpfsysc eaekrksdyi irtlkvkfns |
| 181 | etrtiyqfhy knwpdhdvps sidpileliw dvrcyqedds vpicihcsag cgrtgvicai |
| 241 | dytwmllkdg sqakhcipek nhtlqadsys pnlpksttka akmmnqqrtk meikesssfd |
| 301 | frtseisake elvlhpakss tsfdflelny sfdknadttm kwqtkafpiv geplqkhqsl |
| 361 | dlgsllfegc snskpvnaag ryfnskvpit rtkstpfeli qqretkevds kenfsylesq |
| 421 | phdscfvemq aqkvmhvssa elnyslpyds khqirnasnv khhdssalgv ysyiplvenp |
| 481 | yfsswppsgt sskmsldlpe kqdgtvfpss llptsstslf syynshdsls lnsptnissl |
| 541 | lnqesavlat apriddeipp plpvrtpesf ivveeagefs pnvpkslssa vkvkigtsle |
| 601 | wggtsepkkf ddsvilrpsk svklrspkse lhqdrssppp plpertlesf fladedcmqa |
| 661 | qsietystsy pdtmenstss kqtlktpgks ftrskslkil rnmkksicns cppnkpaesv |
| 721 | qsnnsssfln fgfanrfskp kgprnppptw ni |

The mRNA sequence encoding human TNFAIP3 provided by Genbank Accession No. BC114480.1, is incorporated herein by reference, and is shown below (SEQ ID NO: 191).

| | |
|---|---|
| 1 | ccggagaggt gttggagagc acaatggctg aacaagtcct tcctcaggct ttgtatttga |
| 61 | gcaatatgcg gaaagctgtg aagatacggg agagaactcc agaagacatt tttaaaccta |
| 121 | ctaatgggat cattcatcat tttaaaacca tgcaccgata cacactggaa atgttcagaa |
| 181 | cttgccagtt ttgtcctcag tttcgggaga tcatccacaa agccctcatc gacagaaaca |
| 241 | tccaggccac cctggaaagc cagaagaaac tcaactggtg tcgagaagtc cggaagcttg |
| 301 | tggcgctgaa aacgaacggt gacggcaatt gcctcatgca tgccacttct cagtacatgt |
| 361 | ggggcgttca ggacacagac ttggtactga ggaaggcgct gttcagcacg ctcaaggaaa |
| 421 | cagacacacg caactttaaa ttccgctggc aactggagtc tctcaaatct caggaatttg |
| 481 | ttgaaacggg gctttgctat gatactcgga actggaatga tgaatgggac aatcttatca |
| 541 | aaatggcttc cacagacaca cccatggccc gaagtggact tcagtacaac tcactggaag |
| 601 | aaatacacat atttgtcctt tgcaacatcc tcagaaggcc aatcattgtc atttcagaca |
| 661 | aaatgctaag aagtttggaa tcaggttcca atttcgcccc tttgaaagtg ggtggaattt |
| 721 | acttgcctct ccactggcct gcccaggaat gctacagata ccccattgtt ctcggctatg |
| 781 | acagccatca ttttgtaccc ttggtgaccc tgaaggacag tgggcctgaa atccgagctg |
| 841 | ttccacttgt taacagagac cggggaagat ttgaagactt aaaagttcac tttttgacag |
| 901 | atcctgaaaa tgagatgaag gagaagctct taaaagagta cttaatggtg atagaaatcc |
| 961 | ccgtccaagg ctgggaccat ggcacaactc atctcatcaa tgccgcaaag ttggatgaag |
| 1021 | ctaacttacc aaaagaaatc aatctggtag atgattactt tgaacttgtt cagcatgagt |
| 1081 | acaagaaatg gcaggaaaac agcgagcagg ggaggagaga ggggcacgcc cagaatccca |
| 1141 | tggaaccttc cgtgccccag ctttctctca tggatgtaaa atgtgaaacg cccaactgcc |
| 1201 | ccttcttcat gtctgtgaac acccagcctt tatgccatga gtgctcagag aggcggcaaa |
| 1261 | agaatcaaaa caaactccca aagctgaact ccaagccggg ccctgagggg ctccctggca |
| 1321 | tggcgctcgg ggcctctcgg ggagaagcct atgagccctt ggcgtggaac cctgaggagt |
| 1381 | ccactggggg gcctcattcg gccccaccga cagcacccag cccttttctg ttcagtgaga |
| 1441 | ccactgccat gaagtgcagg agccccggct gccccttcac actgaatgtg cagcacaacg |
| 1501 | gattttgtga acgttgccac aacgcccggc aacttcacgc cagccacgcc ccagaccaca |
| 1561 | caaggcactt ggatcccggg aagtgccaag cctgcctcca ggatgttacc aggacattta |
| 1621 | atgggatctg cagtacttgc ttcaaaagga ctacagcaga ggcctcctcc agcctcagca |
| 1681 | ccagcctccc tccttcctgt caccagcgtt ccaagtcaga tccctcgcgg ctcgtccgga |
| 1741 | gcccctcccc gcattcttgc cacagagctg gaaacgacgc ccctgctggc tgcctgtctc |
| 1801 | aagctgcacg gactcctggg gacaggacgg ggacgagcaa gtgcagaaaa gccggctgcg |
| 1861 | tgtattttgg gactccagaa aacaagggct tttgcacact gtgtttcatc gagtacagag |
| 1921 | aaaacaaaca ttttgctgct gcctcaggga aagtcagtcc cacagcgtcc aggttccaga |
| 1981 | acaccattcc gtgcctgggg agggaatgcg gcacccttgg aagcaccatg tttgaaggat |
| 2041 | actgccagaa gtgtttcatt gaagctcaga atcagagatt tcatgaggcc aaaaggacag |
| 2101 | aagagcaact gagatcgagc cagcgcagag atgtgcctcg aaccacacaa agcacctcaa |
| 2161 | ggcccaagtg cgcccgggcc tcctgcaaga acatcctggc ctgccgcagc gaggagctct |
| 2221 | gcatggagtg tcagcatccc aaccagagga tgggccctgg ggcccaccgg ggtgagcctg |
| 2281 | cccccgaaga cccccccaag cagcgttgcc gggcccccgc ctgtgatcat tttggcaatg |
| 2341 | ccaagtgcaa cggctactgc aacgaatgct ttcagttcaa gcagatgtat ggctaaccgg |
| 2401 | aaacaggtgg gtcacctcct gcaagaagtg gggcctcgag ctgtcagtca tcatggtgct |
| 2461 | atcctctgaa cccctcagct gccactgcaa cagtgggctt aagggtgtct gagcaggaga |
| 2521 | ggaaagataa gctcttcgtg gtgcccacga tgctcaggtt tggtaacccg ggagtgttcc |
| 2581 | caggtggcct tagaaagcaa agcttgtaac tggcaaggga tgatgtcaga ttcagcccaa |
| 2641 | ggttcctcct ctcctaccaa gcaggaggcc aggaacttct ttggacttgg aaggtgtgcg |
| 2701 | gggactggcc gaggcccctg caccctgcgc atcaggactg cttcatcgtc ttggctgaga |
| 2761 | aagggaaaag acacacaagt cgcgtgggtt ggagaagcca gagccattcc acctcccctc |
| 2821 | ccccagcatc tctcagagat gtgaagccag atcctcatgg cagcgaggcc ctctgcaaga |
| 2881 | agctcaagga agctcaggga aaatggacgt attcagagag tgtttgtagt tcatggtttt |
| 2941 | tccctacctg cccggttcct ttcctgagga cccggcagaa atgcagaacc atccatggac |
| 3001 | tgtgattctg aggctgctga gactgaacat gttcacattg acagaaaaac aagctgctct |
| 3061 | ttataatatg caccttttaa aaaattagaa tattttactg ggaagacgtg taactctttg |
| 3121 | ggttattact gtctttactt ctaaagaagt tagcttgaac tgaggagtaa aagtgtgtac |
| 3181 | atatataata tacccttaca ttatgtatga gggatttttt taaattatat tgaaatgctg |
| 3241 | ccctagaagt acaataggaa ggctaaataa taataacctg ttttctggtt gttgttgggg |
| 3301 | catgagcttg tgtatacact gcttgcataa actcaaccag ctgccttttt aaagggagct |
| 3361 | ctagtccttt ttgtgtaatt cactttattt attttattac aaacttcaag attatttaag |
| 3421 | cgaagatatt tcttcagctc tggggaaaat gccacagtgt tctcctgaga gaacatcctt |
| 3481 | gctttgagtc aggctgtggg caagttcctg accacaggga gtaaattggc ctctttgata |
| 3541 | cacttttgct tgcctcccca ggaaagaagg aattgcatcc aaggtataca tacatattca |
| 3601 | tcgatgtttc gtgcttctcc ttatgaaact ccagctatgt aataaaaaac tatactctgt |
| 3661 | gttctgttaa tgcctctgag tgtcctacct ccttggagat gagataggga aggagcaggg |
| 3721 | atgagactgg caatggtcac agggaaagat gtggcctttt gtgatggttt tattttctgt |
| 3781 | taacactgtg tcctgggggg gctgggaagt cccctgcatc ccatg |

The atg start and stop codons are bolded and underlined. The amino acid sequence of human TNFAIP3, provided by Genbank Accession No. AAI14481.1, is incorporated herein by reference, and is shown below (SEQ ID NO: 192).

| | |
|---|---|
| 1 | maeqvlpqal ylsnmrkavk irertpedif kptngiihhf ktmhrytlem frtcqfcpqf |
| 61 | reiihkalid rniqatlesq kklnwcrevr klvalktngd gnclmhatsq ymwgvqdtdl |
| 121 | vlrkalfstl ketdtrnfkf rwqleslksq efvetglcyd trnwndewdn likmastdtp |
| 181 | marsglqyns leeihifylc nilrrpiivi sdkmlisles gsnfaplkvg giylplhwpa |
| 241 | qecyrypivl gydshhfvpl vtlkdsgpei ravplvnrdr grfedlkvhf ltdpenemke |
| 301 | kllkeylmvi eipvqgwdhg tthlinaakl deanlpkein lvddyfelvq heykkwqens |
| 361 | eqgrreghaq npmepsvpql slmdvkcetp ncpffmsvnt qplchecser rqknqnklpk |
| 421 | lnskpgpegl pgmalgasrg eayeplawnp eestggphsa pptapspflf settamkcrs |
| 481 | pgcpftlnvq hngfcerchn arqlhashap dhtrhldpgk cqaclqdvtr tfngicstcf |
| 541 | krttaeasss lstslppsch qrsksdpsrl vrspsphsch ragndapagc lsqaartpgd |
| 601 | rtgtskcrka gcvyfgtpen kgfcticfie yrenkhfaaa sgkvsptasr fqntipclgr |
| 661 | ecgtlgstmf egycqkcfie aqnqrfheak rteeqlrssq advprttqs tsrpkcaras |
| 721 | cknilacrse elcmecqhpn qrmgpgahrg epapedppkq rcrapacdhf gnakcngycn |
| 781 | ecfqfkqmyg |

The mRNA sequence encoding human STAT4 provided by Genbank Accession No. L78440.1, is incorporated herein by reference, and is shown below (SEQ ID NO: 193).

| | |
|---|---|
| 1 | gctttctcct agggactgtg aggggcgctt ctgactttgg acttgagcac tgcctgggac |
| 61 | ctgtgctgag agagcgctag catgtctcag tggaatcaag tccaacagtt agaaatcaag |
| 121 | tttttggagc aggtggatca attctatgat gacaactttc ccatggaaat tcggcatctg |
| 181 | ttggcccaat ggattgaaaa tcaagactgg gaggcagctt ctaacaatga aaccatggca |
| 241 | acgattcttc ttcaaaactt gttaatacaa ctggatgaac agttaggtcg tgtttccaaa |
| 301 | gagaaaaacc tactcttgat acacaatcta aaaagaatta ggaaggtcct tcagggaaaa |
| 361 | tttcatggaa atccaatgca tgtagctgtg gttatttcaa actgtttaag ggaagagagg |
| 421 | agaatattgg ctgcagccaa catgcctgtc caggggcctc tagagaaatc cttacaaagt |
| 481 | tcttcagttt cagaaagaca gaggaatgtg gagcacaaag tggctgccat taaaaacagt |
| 541 | gtgcagatga cagaacaaga taccaaatac ttagaagatc tgcaagacga atttgactac |
| 601 | aggtataaaa caattcagac aatggatcag agtgacaaga atagtgccat ggtgaatcag |
| 661 | gaagttttga cactgcagga aatgcttaac agcctcgatt tcaagagaaa ggaggctctc |
| 721 | agtaaaatga cccaaatcat ccatgagaca gacctgttaa tgaacaccat gctcatagaa |
| 781 | gagctgcaag actggaagcg gcggcagcaa atcgcctgca tcgggggtcc actccacaat |
| 841 | gggctcgacc agcttcagaa ctgctttaca ctattggcag aaagtctttt ccaactgaga |
| 901 | aggcaattgg agaaactaga ggagcaatct accaaaatga catatgaagg tgatcccatt |
| 961 | ccaatgcaaa gaactcacat gctagaaaga gtcaccttct tgatctacaa ccttttcaag |
| 1021 | aactcatttg tggttgagcg acagccatgt atgccaaccc accctcagag gccgttggta |
| 1081 | cttaaaaccc taattcagtt cactgtaaaa ctaaggctac taataaaatt gccagaacta |
| 1141 | aactatcagg taaaggttaa ggcatcaatt gacaagaatg tttcaactct aagcaaccga |
| 1201 | agatttgtac tttgtggaac taatgtcaaa gccatgtcta ttgaagaatc ttccaatggg |
| 1261 | agtctctcag tagaatttcg acatttgcaa ccaaaggaaa tgaagtccag tgctggaggt |
| 1321 | aaaggaaatg agggctgtca catggtgact gaagaacttc attccataac gtttgaaaca |
| 1381 | cagatctgcc tctatggcct gaccatagat ttggagacca gctcattgcc tgtggtgatg |
| 1441 | atttccaatg tcagtcagtt acctaatgct tgggcatcca tcatttggta caacgtgtca |
| 1501 | accaacgatt cccagaactt ggttttcttt aataatcctc cacctgccac attgagtcaa |
| 1561 | ctactggagg tgatgagctg gcagttttca tcgtacgttg gtcgtggtct taactcagat |
| 1621 | caactccata tgctggcaga gaagcttaca gtccaatcta gctacagtga tggtcacctc |
| 1681 | acctgggcca agttctgcaa ggaacattta cctggtaaat catttacctt ttggacatgg |
| 1741 | cttgaagcaa tattggatct aattaagaaa cacattcttc ccctttggat tgatgggtat |
| 1801 | gtcatgggct ttgttagcaa agagaaggaa cggctgttgc taaaggataa aatgcctggc |
| 1861 | acctttttat taagattcag tgaaagccat ctcggaggaa taactttcac ctgggtggac |
| 1921 | cattctgaaa gtggggaagt gagattccac tctgtagaac cctacaataa aggccggttg |
| 1981 | tctgctctgc cattcgctga catcctgcga gactacaaag ttattatggc tgaaaacatt |
| 2041 | cctgaaaacc ctctgaagta cctatatcct gacattccca aagacaaagc cttcggtaaa |
| 2101 | cactacagct ctcagccttg cgaagtttca agaccaacag aaaggggtga caaaggttat |
| 2161 | gttccttctg tttttatccc catctcaaca atccgaagtg attcaacaga gccacattct |
| 2221 | ccatcagacc ttcttcccat gtctccaagt gtgtatgcgg tgttgagaga aaacctgagt |
| 2281 | cccacaacaa ttgaaactgc aatgaagtct ccttattctg ctgaatgaca ggataaactc |
| 2341 | tgacgcacca agaaaggaag caaatgaaaa agtttaaaga ctgttctttg cccaataacc |
| 2401 | acattttatt tcttcagctt tgtaaatacc aggttctagg aaatgtttga catctgaagc |
| 2461 | tctcttcaca ctcccgtggc actcctcaat tgggagtgtt gtgactgaaa tgcttgaaac |
| 2521 | caaagcttca gataaacttg caagataaga caactttaag aaaccagtgt taataacaat |
| 2581 | attaacag |

The atg start and stop codons are bolded and underlined. The amino acid sequence of human STAT4, provided by Genbank Accession No. AAB05605.1, is incorporated herein by reference, and is shown below (SEQ ID NO: 194).

| | |
|---|---|
| 1 | msqwnqvqql eikfleqvdq fyddnfpmei rhllaqwien qdweaasnne tmatillqnl |
| 61 | liqldeqlgr vskeknllli hnlkrirkvl qgkfhgnpmh vawisnclr eerrilaaan |
| 121 | mpvqgpleks lqsssyserq rnvehkvaai knsvqmteqd tkyledlqde fdyryktiqt |
| 181 | mdqsdknsam vnqevltlqe mlnsldfkrk ealskmtqii hetdllmntm lieelqdwkr |
| 241 | rqqiaciggp lhngldqlqn cftllaeslf qlrrqlekle eqstkmtyeg dpipmqrthm |
| 301 | lervtfliyn lfknsfvver qpcmpthpqr plvlktliqf tvklrllikl pelnyqvkvk |
| 361 | asidlmvstl snrrfvlcgt nvkamsiees sngslsvefr hlqpkemkss aggkgnegch |
| 421 | mvteelhsit fetqiclygl tidletsslp vvmisnvsql pnawasiiwy nvstndsqn1 |
| 481 | vffnnpppat lsqllevmsw qfssyvgrgl nsdqlhmlae kltvqssysd ghltwakfck |
| 541 | ehlpgksftf wtwleaildl ikkhilplwi dgyvmgfvsk ekerlllkdk mpgtfllrfs |
| 601 | eshlggitft wvdhsesgev rfhsvepynk grlsalpfad ilrdykvima enipenpk |
| 661 | lypdipkdka fgkhyssqpc evsrptergd kgyvpsvfip istirsdste phspsdllpm |
| 721 | spsvyavlre nlspttieta mkspysae |

The mRNA sequence encoding human CCR6 provided by Genbank Accession No. AY242126.1, is incorporated herein by reference, and is shown below (SEQ ID NO: 195).

| | |
|---|---|
| 1 | atgagcgggg aatcaatgaa tttcagcgat gttttcgact ccagtgaaga ttattttgtg |
| 61 | tcagtcaata cttcatatta ctcagttgat tctgagatgt tactgtgctc cttgcaggag |
| 121 | gtcaggcagt tctccaggct atttgtaccg attgcctact ccttgatctg tgtctttggc |
| 181 | ctcctgggga atattctggt ggtgatcacc Mgcttttt ataagaaggc caggtctatg |
| 241 | acagacgtct atctcttgaa catggccatt gcagacatcc tctttgttct tactctccca |
| 301 | ttctgggcag tgagtcatgc cactggtgcg tgggttttca gcaatgccac gtgcaagttg |
| 361 | ctaaaaggca tctatgccat caactttaac tgcgggatgc tgctcctgac ttgcattagc |
| 421 | atggaccggt acatcgccat tgtacaggcg actaagtcat tccggctccg atccagaaca |
| 481 | ctaccgcgca gcaaaatcat ctgccttgtt gtgtgggggc tgtcagtcat catctccagc |
| 541 | tcaacttttg tcttcaacca aaaatacaac acccaaggca gcgatgtctg tgaacccaag |
| 601 | taccagactg tctcggagcc catcaggtgg aagctgctga tgttggggct tgagctactc |
| 661 | tttggtttct ttatcccttt gatgttcatg atattttgtt acacgttcat tgtcaaaacc |
| 721 | ttggtgcaag ctcagaattc taaaaggcac aaagccatcc gtgtaatcat agctgtggtg |
| 781 | cttgtgtttc tggcttgtca gattcctcat aacatggtcc tgcttgtgac ggctgcaaat |
| 841 | ttgggtaaaa tgaaccgatc ctgccagagc gaaaagctaa ttggctatac gaaaactgtc |
| 901 | acagaagtcc tggctttcct gcactgctgc ctgaaccctg tgctctacgc ttttattggg |
| 961 | cagaagttca gaaactactt tctgaagatc ttgaaggacc tgtggtgtgt gagaaggaag |
| 1021 | tacaagtcct caggcttctc ctgtgccggg aggtactcag aaaacatttc tcggcagacc |
| 1081 | agtgagaccg cagataacga caatgcgtcg tccttcacta tgtga |

The atg start and stop codons are bolded and underlined. The amino acid sequence of human CCR6, provided by Genbank Accession No. AA092293.1, is incorporated herein by reference, and is shown below (SEQ ID NO: 196).

| | |
|---|---|
| 1 | msgesmnfsd vfdssedyfv svntsyysvd semllcslqe vrqfsrlfvp iayslicvfg |
| 61 | llgnilvvit fafykkarsm tdvyllnmai adilfvltlp fwavshatga wvfsnatckl |
| 121 | lkgiyainfn cgmllltcis mdryiaivqa tksfrlisrt lprskiiclv vwglsviiss |
| 181 | stfvfnqkyn tqgsdvcepk yqtvsepirw kllmlglell fgffiplmfm ifcytfivkt |
| 241 | lvqaqnskrh kairviiavv lvflacqiph nmvllvtaan lgkmnrscqs ekligytktv |
| 301 | tevlaflhcc lnpvlyafig qkfrnyflki lkdlwcyrrk ykssgfscag rysenisrqt |
| 361 | setadndnas sftm |

The mRNA sequence encoding human TNFR-1 (tumor necrosis factor receptor 1) provided by Genbank Accession No. NM_001065.3, is incorporated herein by reference, and is shown below (SEQ ID NO: 197).

| | |
|---|---|
| 1 | ctcctccagc tcttcctgtc ccgctgttgc aacactgcct cactcttccc ctcccacctt |
| 61 | ctctcccctc ctctctgctt taattttctc agaattctct ggactgaggc tccagttctg |
| 121 | gcctttgggg ttcaagatca ctgggaccag gccgtgatct ctatgcccga gtctcaaccc |
| 181 | tcaactgtca ccccaaggca cttgggacgt cctggacaga ccgagtcccg ggaagcccca |
| 241 | gcactgccgc tgccacactg ccctgagccc aaatggggga gtgagaggcc atagctgtct |
| 301 | ggcatgggcc tctccaccgt gcctgacctg ctgctgccac tggtgctcct ggagctgttg |
| 361 | gtgggaatat acccctcagg ggttattgga ctggtccctc acctagggga cagggagaag |
| 421 | agagatagtg tgtgtcccca aggaaaatat atccaccctc aaaataattc gatttgctgt |
| 481 | accaagtgcc acaaaggaac ctacttgtac aatgactgtc caggcccggg gcaggatacg |
| 541 | gactgcaggg agtgtgagag cggctccttc accgcttcag aaaaccacct cagacactgc |
| 601 | ctcagctgct ccaaatgccg aaaggaaatg ggtcaggtgg agatctcttc ttgcacagtg |
| 661 | gaccgggaca ccgtgtgtgg ctgcaggaag aaccagtacc ggcattattg gagtgaaaac |
| 721 | cttttccagt gcttcaattg cagcctctgc ctcaatggga ccgtgcacct ctcctgccag |
| 781 | gagaaacaga acaccgtgtg cacctgccat gcaggtttct ttctaagaga aaacgagtgt |
| 841 | gtctcctgta gtaactgtaa gaaaagcctg gagtgcacga agttgtgcct accccagatt |
| 901 | gagaatgtta agggcactga ggactcaggc accacagtgc tgttgcccct ggtcattttc |
| 961 | tttggtcttt gccttttatc cctcctcttc attggtttaa tgtatcgcta ccaacggtgg |
| 1021 | aagtccaagc tctactccat tgtttgtggg aaatcgacac ctgaaaaaga gggggagctt |
| 1081 | gaaggaacta ctactaagcc cctggcccca aacccaagct tcagtcccac tccaggcttc |
| 1141 | acccccaccc tgggcttcag tcccgtgccc agttccacct tcacctccag ctccacctat |
| 1201 | acccccggtg actgtcccaa ctttgcggct ccccgcagag aggtggcacc accctatcag |
| 1261 | ggggctgacc ccatccttgc gacagccctc gcctccgacc ccatccccaa cccccttcag |
| 1321 | aagtgggagg acagcgccca caagccacag agcctagaca ctgatgaccc cgcgacgctg |
| 1381 | tacgccgtgg tggagaacgt gcccccgttg cgctggaagg aattcgtgcg gcgcctaggg |
| 1441 | ctgagcgacc acgagatcga tcggctggag ctgcagaacg ggcgctgcct gcgcgaggcg |
| 1501 | caatacagca tgctggcgac ctggaggcgg cgcacgccgc ggcgcgaggc cacgctggag |
| 1561 | ctgctgggac gcgtgctccg cgacatggac ctgctgggct gcctggagga catcgaggag |
| 1621 | gcgctttgcg gccccgccgc cctcccgccc gcgcccagtc ttctcagatg aggctgcgcc |
| 1681 | cctgcgggca gctctaagga ccgtcctgcg agatcgcctt ccaaccccac ttttttctgg |
| 1741 | aaaggagggg tcctgcaggg gcaagcagga gctagcagcc gcctacttgg tgctaacccc |
| 1801 | tcgatgtaca tagcttttct cagctgcctg cgcgccgccg acagtcagcg ctgtgcgcgc |
| 1861 | ggagagaggt gcgccgtggg ctcaagagcc tgagtgggtg gtttgcgagg atgagggacg |
| 1921 | ctatgcctca tgcccgtttt gggtgtcctc accagcaagg ctgctcgggg gcccctggtt |
| 1981 | cgtccctgag cctttttcac agtgcataag cagttttttt tgtttttgtt ttgttttgtt |
| 2041 | ttgtttttaa atcaatcatg ttacactaat agaaacttgg cactcctgtg ccctctgcct |
| 2101 | ggacaagcac atagcaagct gaactgtcct aaggcagggg cgagcacgga acaatggggc |
| 2161 | cttcagctgg agctgtggac ttttgtacat acactaaaat tctgaagtta aagctctgct |
| 2221 | cttggaaaaa aaaaaaaaaa aaaaaaaaaa aaaaaaaa |

The atg start and stop codons are bolded and underlined. The amino acid sequence of human TNFR-1 (tumor necrosis factor receptor 1), provided by Genbank Accession No. NP_001056.1, is incorporated herein by reference, and is shown below (SEQ ID NO: 198).

| | |
|---|---|
| 1 | mglstvpdll lplvllellv giypsgvigl vphlgdrekr dsvcpqgkyi hpqnnsicct |
| 61 | kchkgtylyn dcpgpgqdtd crecesgsft asenhlrhcl scskcrkemg qveissctvd |
| 121 | rdtvcgcrkn qyrhywsenl fqcfncslcl ngtvhlscqe kqntvctcha gfflrenecv |
| 181 | scsnckksle ctklclpqie nvkgtedsgt tvllplviff glcllsllfi glmyryqrwk |
| 241 | sklysivcgk stpekegele gtttkplapn psfsptpgft ptlgfspvps stftssstyt |
| 301 | pgdcpnfaap rrevappyqg adpilatala sdpipnplqk wedsahkpqs ldtddpatly |
| 361 | avvenvpplr wkefvrrlgl sdheidrlel qngrclreaq ysmlatwrrr tprreatlel |
| 421 | lgrvlrdmdl lgcledieea lcgpaalppa psllr |

The mRNA sequence encoding human TNFR-2 provided by Genbank Accession No. M55994.1, is incorporated herein by reference, and is shown below (SEQ ID NO: 199).

| | |
|---|---|
| 1 | gaattcggcg cagcggagcc tggagagaag gcgctgggct gcgagggcgc gagggcgcga |
| 61 | gggcaggggg caaccggacc ccgcccgcac ccatggcgcc cgtcgccgtc tgggccgcgc |
| 121 | tggccgtcgg actggagctc tgggctgcgg cgcacgcctt gcccgcccag gtggcattta |
| 181 | caccctacgc cccggagccc gggagcacat gccggctcag agaatactat gaccagacag |
| 241 | ctcagatgtg ctgcagcaag tgctcgccgg gccaacatgc aaaagtcttc tgtaccaaga |
| 301 | cctcggacac cgtgtgtgac tcctgtgagg acagcacata cacccagctc tggaactggg |
| 361 | ttcccgagtg cttgagctgt ggctcccgct gtagctctga ccaggtggaa actcaagcct |
| 421 | gcactcggga acagaaccgc atctgcacct gcaggcccgg ctggtactgc gcgctgagca |
| 481 | agcaggaggg gtgccggctg tgcgcgccgc tgcgcaagtg ccgcccgggc ttcggcgtgg |
| 541 | ccagaccagg aactgaaaca tcagacgtgg tgtgcaagcc ctgtgccccg gggacgttct |
| 601 | ccaacacgac ttcatccacg gatatttgca ggccccacca gatctgtaac gtggtggcca |
| 661 | tccctgggaa tgcaagcagg gatgcagtct gcacgtccac gtcccccacc cggagtatgg |
| 721 | ccccaggggc agtacactta ccccagccag tgtccacacg atcccaacac acgcagccaa |
| 781 | ctccagaacc cagcactgct ccaagcacct ccttcctgct cccaatgggc cccagccccc |
| 841 | cagctgaagg gagcactggc gacttcgctc ttccagttgg actgattgtg ggtgtgacag |
| 901 | ccttgggtct actaataata ggagtggtga actgtgtcat catgacccag gtgaaaaaga |
| 961 | agcccttgtg cctgcagaga gaagccaagg tgcctcactt gcctgccgat aaggcccggg |
| 1021 | gtacacaggg ccccgagcag cagcacctgc tgatcacagc gccgagctcc agcagcagct |
| 1081 | ccctggagag ctcggccagt gcgttggaca gaagggcgcc cactcggaac cagccacagg |
| 1141 | caccaggcgt ggaggccagt ggggccgggg aggcccgggc cagcaccggg agctcagatt |
| 1201 | cttcccctgg tggccatggg acccaggtca atgtcacctg catcgtgaac gtctgtagca |
| 1261 | gctctgacca cagctcacag tgctcctccc aagccagctc cacaatggga gacacagatt |
| 1321 | ccagcccctc ggagtccccg aaggacgagc aggtcccctt ctccaaggag gaatgtgcct |
| 1381 | ttcggtcaca gctggagacg ccagagaccc tgctggggag caccgaagag aagcccctgc |
| 1441 | cccttggagt gcctgatgct gggatgaagc ccagttaacc aggccggtgt gggctgtgtc |
| 1501 | gtagccaagg tgggctgagc cctggcagga tgaccctgcg aaggggccct ggtccttcca |
| 1561 | ggcccccacc actaggactc tgaggctctt tctgggccaa gttcctctag tgccctccac |
| 1621 | agccgcagcc tccctctgac ctgcaggcca agagcagagg cagcgggttg tggaaagcct |
| 1681 | ctgctgccat ggtgtgtccc tctcggaagg ctggctgggc atggacgttc ggggcatgct |
| 1741 | ggggcaagtc cctgactctc tgtgacctgc cccgcccagc tgcacctgcc agcctggctt |
| 1801 | ctggagccct tgggtttttt gtttgtttgt ttgtttgttt gtttgtttct ccccctgggc |
| 1861 | tctgccccag ctctggcttc cagaaaaccc cagcatcctt ttctgcagag gggctttctg |
| 1921 | gagaggaggg atgctgcctg agtcacccat gaagacagga cagtgcttca gcctgaggct |
| 1981 | gagactgcgg gatggtcctg gggctctgtg cagggaggag gtggcagccc tgtagggaac |
| 2041 | ggggtccttc aagttagctc aggaggcttg gaaagcatca cctcaggcca ggtgcagtcc |
| 2101 | ctcacgccta tgatcccagc actttgggag gctgaggcgg gtggatcacc tgaggttagg |
| 2161 | agttcgagac cagcctggcc aacatggtaa aaccccatct ctactaaaaa tacagaaatt |
| 2221 | agccgggcgt ggtggcgggc acctatagtc ccagctactc agaagcctga ggctgggaaa |
| 2281 | tcgtttgaac ccgggaagcg gaggttgcag ggagccgaga tcacgccact gcactccagc |
| 2341 | ctgggcgaca gagcgagagt ctgtctcaaa agaaaaaaaa aaaaaaccga attc |

The atg start and stop codons are bolded and underlined. The amino acid sequence of human TNFR-2, provided by Genbank Accession No. AAA36755.1, is incorporated herein by reference, and is shown below (SEQ ID NO: 200).

| | |
|---|---|
| 1 | mapvavwaal avglelwaaa halpaqvaft pyapepgstc rlreyydqta qmccskcspg |
| 61 | qhakvfctkt sdtvcdsced stytqlwnwv peclscgsrc ssdqvetqac treqnrictc |
| 121 | rpgwycalsk qegcrlcapl rkcrpgfgva rpgtetsdvv ckpcapgtfs nttsstdicr |
| 181 | phqicnvvai pgnasrdavc tstsptrsma pgavhlpqpv strsqhtqpt pepstapsts |
| 241 | fllpmgpspp aegstgdfal pvglivgvta lglliigvvn cvimtqvkkk plclqreakv |
| 301 | phlpadkarg tqgpeqqhll itapssssss lessasaldr raptrnqpqa pgveasgage |
| 361 | arastgssds spgghgtqvn vtcivnvcss sdhssqcssq asstmgdtds spsespkdeq |
| 421 | vpfskeecaf rsqletpetl lgsteekplp lgvpdagmkp s |

(Signall peptide AA 1-22; mature peptide AA 23-461).

The mRNA sequence encoding human cell death protein (RIP) provided by Genbank Accession No. U25994.1, is incorporated herein by reference, and is shown below (SEQ ID NO: 201).

| | |
|---|---|
| 1 | gacgtgaaga gtttaaagaa agagtattca aacgaaaatg cagttgtgaa gagaatgcag |
| 61 | tctcttcaac ttgattgtgt ggcagtacct tcaagccggt caaattcagc cacagaacag |
| 121 | cctggttcac tgcacagttc ccagggactt gggatgggtc ctgtggagga gtcctggttt |
| 181 | gctccttccc tggagcaccc acaagaagag aatgagccca gcctgcagag taaactccaa |
| 241 | gacgaagcca actaccatct ttatggcagc cgcatggaca ggcagacgaa acagcagccc |
| 301 | agacagaatg tggcttacaa cagagaggag gaaaggagac gcagggtctc ccatgaccct |
| 361 | tttgcacagc aaagacctta cgagaatttt cagaatacag agggaaaagg cactgtttat |
| 421 | tccagtgcag ccagtcatgg taatgcagtg caccagccat cagggctcac cagccaacct |
| 481 | caagtactgt atcagaacaa tggattatat agctcacatg gctttggaac aagaccactg |
| 541 | gatccaggaa cagcaggtcc cagagtttgg tacaggccaa ttccaagtca tatgcctagt |
| 601 | ctgcataata tcccagtgcc tgagaccaac tatctaggaa attctcccac catgccattc |
| 661 | agctccttgc caccaacaga tgaatctata aaatatacca tatacaatag tactggcatt |
| 721 | cagattggag cctacaatta tatggagatt ggtgggacga gttcatcact actagacagc |
| 781 | acaaatacga acttcaaaga agagccagct gctaagtacc aagctatctt tgataatacc |
| 841 | actagtctga cggataaaca cctggaccca atcagggaaa atctgggaaa gcactggaaa |
| 901 | aactgtgccc gtaaactggg cttcacacag tctcagattg atgaaattga ccatgactat |
| 961 | gagcgagatg gactgaaaga aaaggtttac cagatgctcc aaaagtgggt gatgagggaa |
| 1021 | ggcataaagg gagccacggt ggggaagctg gcccaggcgc tccaccagtg ttccaggatc |
| 1081 | gaccttctga gcagcttgat ttacgtcagc cagaactaac cctggatggg ctacggcagc |
| 1141 | tgaagtggac gcctcactta gtggataacc ccagaaagtt ggctgcctca gagcattcag |
| 1201 | aattctgtcc tcactgatag gggttctgtg tctgcagaaa |

The atg start and stop codons are bolded and underlined. The amino acid sequence of human RIP, provided by Genbank Accession No. AAC50137.1, is incorporated herein by reference, and is shown below (SEQ ID NO: 202).

| | |
|---|---|
| 1 | dvkslkkeys nenavvkrmq slqldcvavp ssrsnsateq pgslhssqgl gmgpveeswf |
| 61 | apslehpqee nepslqsklq deanyhlygs rmdrqtkqqp rqnvaynree errrrvshdp |
| 121 | faqqrpyenf qntegkgtvy ssaashgnav hqpsgltsqp qvlyqnngly sshgfgtrpl |
| 181 | dpgtagprvw yrpipshmps lhnipvpetn ylgnsptmpf sslpptdesi kytiynstgi |
| 241 | qigaynymei ggtsssllds tntnfkeepa akyqaifdnt tsltdkhldp irenlgkhwk |
| 301 | ncarklgftq sqideidhdy erdglkelwy qmlqkwvmre gikgatvgkl aqalhqcsri |
| 361 | dllssliyvs qn |

The mRNA sequence encoding human TRADD provided by Genbank Accession No. NM_003789.3, is incorporated herein by reference, and is shown below (SEQ ID NO: 203).

| | |
|---|---|
| 1 | gcacacccgg aagcggcgga gtagagcgga gcctggcggg cgtgggaacc caggccccgc |
| 61 | cgaggcggcc aggaggtgag atggcagctg ggcaaaatgg gcacgaagag tgggtgggca |
| 121 | gcgcatacct gtttgtggag tcctcgctgg acaaggtggt cctgtcggat gcctacgcgc |
| 181 | acccccagca gaaggtggca gtgtacaggg ctctgcaggc tgccttggca gagagcggcg |
| 241 | ggagcccgga cgtgctgcag atgctgaaga tccaccgcag cgacccgcag ctgatcgtgc |
| 301 | agctgcgatt ctgcgggcgg cagccctgtg gccgcttcct ccgcgcctac cgcgaggggg |
| 361 | cgctgcgcgc cgcgctgcag aggagcctgg cggccgcgct cgcccagcac tcggtgccgc |
| 421 | tgcaactgga gctgcgcgcc ggcgccgagc ggctggacgc tttgctggcg gacgaggagc |
| 481 | gctgtttgag ttgcatccta gcccagcagc ccgaccggct ccgggatgaa gaactggctg |
| 541 | agctggagga tgcgctgcga aatctgaagt gcggctcggg ggcccggggt ggcgacgggg |
| 601 | aggtcgcttc ggcccccttg cagcccccgg tgccctctct gtcggaggtg aagccgccgc |
| 661 | cgccgccgcc acctgcccag acttttctgt tccagggtca gcctgtagtg aatcggccgc |
| 721 | tgagcctgaa ggaccaacag acgttcgcgc gctctgtggg tctcaaatgg cgcaaggtgg |
| 781 | ggcgctcact gcagcgaggc tgccgggcgc tgcgggaccc ggcgctggac tcgctggcct |
| 841 | acgagtacga gcgcgaggga ctgtacgagc aggccttcca gctgctgcgg cgcttcgtgc |
| 901 | aggccgaggg ccgccgcgcc acgctgcagc gcctggtgga ggcactcgag gagaacgagc |
| 961 | tcaccagcct ggcagaggac ttgctgggcc tgaccgatcc caatggcggc ctggcctaga |
| 1021 | ccaggggtgc agccagcttt tggagaacct ggatggcctt agggttcctt ctgcggctat |
| 1081 | tgctgaaccc ctgtccatcc acgggaccct gaaactccac ttggcctatc tgctggacct |
| 1141 | gctggggcag agttgattgc cttccccagg agccagacca ctgggggtgc atcattgggg |
| 1201 | attctgcctc aggtactttg atagagtgtg gggtgggggg gacctgcttt ggagatcagc |
| 1261 | ctcaccttct cccatcccag aagcggggct tacagccagc ccttacagtt tcactcatga |
| 1321 | agcaccttga tctttggtgt cctggacttc atcctgggtg ctgcagatac tgcagtgaag |
| 1381 | taaaacagga atcaatcttg cctgccccca gctcacactc agcgtgggac cccgaatgtt |
| 1441 | aagcaatgat aataaagtat aacacggatt ttgatgtgag aaaaaaaaaa aaaaaa |

The atg start and stop codons are bolded and underlined. The amino acid sequence of human TRADD, provided by Genbank Accession No. NP_00370.1, is incorporated herein by reference, and is shown below (SEQ ID NO: 204).

| | |
|---|---|
| 1 | maagqnghee wvgsaylfve ssldlwvlsd ayahpqqkva vyralqaala esggspdvlq |
| 61 | mlkihrsdpq livqlrfcgr qpcgrflray regalraalq rslaaalaqh svplqlelra |
| 121 | gaerldalla deerclscil aqqpdrlide elaeledalr nlkcgsgarg gdgevasapl |
| 181 | qppvpslsev kpppppppaq tflfqgqpvv nrplslkdqq tfarsvglkw rkvgrslqrg |
| 241 | cralrdpald slayeyereg lyeqafqllr rfvqaegrra tlqrlveale eneltslaed |
| 301 | llgltdpngg la |

The mRNA sequence encoding human PADI2 (protein-arginine deiminase type-2) provided by Genbank Accession No. NM_007365.2, is incorporated herein by reference, and is shown below (SEQ ID NO: 205).

| | |
|---|---|
| 1 | gcaggctgct ggagaaggcg cacctgctgc aggtgctccc ggccgccccg gaccagcgag |
| 61 | cgcgggcact gcggcgggga ggatgctgcg cgagcggacc gtgcggctgc agtacgggag |
| 121 | ccgcgtggag gcggtgtacg tgctgggcac ctacctctgg accgatgtct acagcgcggc |
| 181 | cccagccggg gcccaaacct tcagcctgaa gcactcggaa cacgtgtggg tggaggtggt |
| 241 | gcgtgatggg gaggctgagg aggtggccac caatggcaag cagcgctggc ttctctcgcc |
| 301 | cagcaccacc ctgcgggtca ccatgagcca ggcgagcacc gaggccagca gtgacaaggt |
| 361 | caccgtcaac tactatgacg aggaagggag cattcccatc gaccaggcgg ggctcttcct |
| 421 | cacagccatt gagatctccc tggatgtgga cgcagaccgg gatggtgtgg tggagaagaa |
| 481 | caacccaaag aaggcatcct ggacctgggg ccccgagggc cagggggcca tcctgctggt |
| 541 | gaactgtgac cgagagacac cctggttgcc caaggaggac tgccgtgatg agaaggtcta |
| 601 | cagcaaggaa gatctcaagg acatgtccca gatgatcctg cggaccaaag gccccgaccg |
| 661 | cctccccgcc ggatacgaga tagttctgta catttccatg tcagactcag acaaagtggg |
| 721 | cgtgttctac gtggagaacc cgttcttcgg ccaacgctat atccacatcc tgggccggcg |
| 781 | gaagctctac catgtggtca agtacacggg tggctccgcg gagctgctgt tcttcgtgga |
| 841 | aggcctctgt ttccccgacg agggcttctc aggcctggtc tccatccatg tcagcctgct |
| 901 | ggagtacatg gcccaggaca ttcccctgac tcccatcttc acggacaccg tgatattccg |
| 961 | gattgctccg tggatcatga cccccaacat cctgcctccc gtgtcggtgt ttgtgtgctg |
| 1021 | catgaaggat aattacctgt tcctgaaaga ggtgaagaac cttgtggaga aaaccaactg |
| 1081 | tgagctgaag gtctgcttcc agtacctaaa ccgaggcgat cgctggatcc aggatgaaat |
| 1141 | tgagtttggc tacatcgagg ccccccataa aggcttcccc gtggtgctgg actctccccg |
| 1201 | agatggaaac ctaaaggact tccctgtgaa ggagctcctg ggcccagatt ttggctacgt |
| 1261 | gacccgggag cccctctttg agtctgtcac cagccttgac tcatttggaa acctggaggt |
| 1321 | cagtccccca gtgaccgtga acggcaagac atacccgctt ggccgcatcc tcatcgggag |
| 1381 | cagctttcct ctgtctggtg gtcggaggat gaccaaggtg gtgcgtgact tcctgaaggc |
| 1441 | ccagcaggtg caggcgcccg tggagctcta ctcagactgg ctgactgtgg gccacgtgga |
| 1501 | tgagttcatg tcctttgtcc ccatccccgg cacaaagaaa ttcctgctac tcatggccag |
| 1561 | cacctcggcc tgctacaagc tcttccgaga gaagcagaag gacggccatg gagaggccat |
| 1621 | catgttcaaa ggcttgggtg ggatgagcag caagcgaatc accatcaaca agattctgtc |
| 1681 | caacgagagc cttgtgcagg agaacctgta cttccagcgc tgcctagact ggaaccgtga |
| 1741 | catcctcaag aaggagctgg gactgacaga gcaggacatc attgacctgc ccgctctgtt |
| 1801 | caagatggac gaggaccacc gtgccagagc cttcttccca aacatggtga acatgatcgt |
| 1861 | gctggacaag gacctgggca tccccaagcc attcgggcca caggttgagg aggaatgctg |
| 1921 | cctggagatg cacgtgcgtg gcctcctgga gcccctgggc ctcgaatgca ccttcatcga |
| 1981 | cgacatttct gcctaccaca aatttctggg ggaagtccac tgtggcacca acgtccgcag |
| 2041 | gaagcccttc accttcaagt ggtggcacat ggtgccctga cctgccaggg gccctggcgt |
| 2101 | ttgcctcctt cgcttagttc tccagaccct ccctcacacg cccagagcct tctgctgaca |
| 2161 | tggactggac agccccgctg ggagaccttt gggacgtggg gtggaatttg gggtatctgt |
| 2221 | gccttgccct ccctgagagg ggcctcagtg tcctctgaag ccatccccag tgagcctcga |
| 2281 | ctctgtccct gctgaaaata gctgggccag tgtctctgta gccctgacat aaggaacaga |
| 2341 | acacaacaaa acacagcaaa ccatgtgccc aaactgctcc ccaaagaatt ttgagtctct |
| 2401 | aatctgacac tgaatgaggg gagaagggaa ggagattctg ggattgccag ttcttccagc |
| 2461 | agccatgctc tgaaaatcaa ggtagaatcc atggaaaggg accccaggac cccgggaccc |
| 2521 | tagacgtatc ttgaactgcc atcgtcattt caaatacatc tccctcaggg tttccaggtg |
| 2581 | gccaccccca attattcatt ccttaccaac ctctcaaatc ctcttggctt tctctctgca |
| 2641 | gtgtggacac tgttggctag tcctccccac tccctgaggg tccagtaagt tagcttagaa |
| 2701 | ccttcctgga aacatttcat ctgagcaggt ttccccacgt gtgggatgct ccttttgcct |
| 2761 | catctgtctc agggatgcag gctcccccgc atgcatgggg atttctcccc agaccagcat |
| 2821 | acttgtgacc tgagagttca atgcgtaaag atgcccctgg tcagccatat ccatcttctc |
| 2881 | ttgcctggtc cttgattctc tggccgctcc ctgaccttcc tccttccact gccttgactt |
| 2941 | tcttcctttt tattcctggt gccatctgtc caggcagcta gacaagaact tgttcgccag |
| 3001 | cagccagatt caggccttcc caggggcata ataagtgacc agcccctcct ctccggacat |
| 3061 | cagatccaac acataaggac cctggcctac cctccagccc aacagccagt tctgggtcag |
| 3121 | ctgccaactt aggggtggtt tgattatccc attgaaattc accagtgcct ttgccaaaga |
| 3181 | ccctctcatt tggacatacc cagattcatt ccctggctcc aactgaaaag actcagtttc |
| 3241 | aatcgttaaa agttccttta gggccagaag aataaatgaa ttataatccc attttgaaga |
| 3301 | accgatttat aaccaatgaa aaggttataa tgtaatttat attcttggag gaacaagatt |
| 3361 | ttcatttggg attatttcct tcaaccattc aacaaacatt tgttgtatgc cactaagcgc |
| 3421 | caggcacggc gttgggctct gcaaacacag tggttagtag cagtctggac ctggtcccta |
| 3481 | ctggcatgga acccatcact ccccaacatg caaagcccac atttaaaggc cagcctctgc |
| 3541 | cccttcagtg atgcgctctt tagaaatgcc agtccactat attcagaaat ccgcagggca |
| 3601 | caaaacttcc agcaagtcac tgttgtggtg aaatgggcag tgggggtggg gggtcttctt |
| 3661 | taaacaggcc cccttcccat ctacctagcc agtacccatc caatgagtcc ccagagcctc |
| 3721 | cagaagctgt tgtctcctct ctggggacag cagctcctgc ctttggaggc caaagcccca |
| 3781 | gatctctcca gccccagagc tgaaaacacc aagtgcctat ttgagggtgt ctgtctggag |
| 3841 | acttagagtt tgtcatgtgt gtgtgtgtgt ttggttaatg tgggtttatg ggttttcttt |
| 3901 | cttttttttc tttttttttt tagtctacat tagggggaag tgagcgcctc ccatgtgcag |
| 3961 | acagtgtgtc tttatagatt tttctaaggc tttccccaat gatgtcggta atttctgatg |
| 4021 | tttctgaagt tcccaggact cacacacccg ttcccatctc acttgcccac ccagtgtgac |
| 4081 | aaccctcggt gtggatatac ccccgtggac tcatggctct tccccacccc cactttctat |
| 4141 | aaatgtaggc ctagaatacg cttctctgtt gcaaaactca gctaagttcc tgcttccacc |
| 4201 | ttgatgttga aatatcttat gtaagagggc aggggatgtc gtgaagatgg caagaagaac |
| 4261 | acagtttcaa atttctggaa aagagcctgt ggtggagatc taaagatgtt tagggaagag |
| 4321 | ctcgactaaa gaacaatgaa ataaatggtc caaggggaag tca |

The atg start and stop codons are bolded and underlined. The amino acid sequence of human PADI2 (protein-arginine deiminase type-2), provided by Genbank Accession No. NP_031391.2, is incorporated herein by reference, and is shown below (SEQ ID NO: 206).

| | |
|---|---|
| 1 | mlrertvrlq ygsrveavyv lgtylwtdvy saapagaqtf slkhsehvwv evvrdgeaee |
| 61 | vatngkqrwl lspsttlrvt msqasteass dkvtvnyyde egsipidqag lfltaieisl |
| 121 | dvdadrdgvv eknnpkkasw twgpegqgai llvncdretp wlpkedcrde lwyskedlkd |
| 181 | msqmilrtkg pdrlpagyei vlyismsdsd kvgvfyvenp ffgqiyihil grrklyhvvk |
| 241 | ytggsaellf fveglcfpde gfsglvsihv slleymaqdi pltpiftdtv ifriapwimt |
| 301 | pnilppvsvf vccmkdnylf lkevknlvek tncelkvcfq ylnrgdrwiq deiefgyiea |
| 361 | phkgfpvvld sprdgnlkdf pvkellgpdf gyvtreplfe svtsldsfgn levsppvtvn |
| 421 | gktyplgril igssfplsgg ilmtkvvrdf lkaqqvqapv elysdwltvg hvdefmsfvp |
| 481 | ipgtkkflll mastsacykl frekqkdghg eaimfkglgg msskritink ilsneslvqe |
| 541 | nlyfqrcldw nrdilkkelg lteqdiidlp alfkmdedhr araffpnmvn mivldkdlgi |
| 601 | pkpfgpqvee ecclemhvrg lleplglect fiddisayhk flgevhcgtn vrrkpftfkw |
| 661 | whmvp |

The mRNA sequence encoding human PAD3 (PADI3) provided by Genbank Accession No. NM_016233.2, is incorporated herein by reference, and is shown below (SEQ ID NO: 207).

| | |
|---|---|
| 1 | agtgttgggg ttggcggcca cagctaagtc caacaccagc atgtcgctgc agagaatcgt |
| 61 | gcgtgtgtcc ctggagcatc ccaccagcgc ggtgtgtgtg gctggcgtgg agaccctcgt |
| 121 | ggacatttat gggtcagtgc ctgagggcac agaaatgttt gaggtctatg ggacgcctgg |
| 181 | cgtggacatc tacatctctc ccaacatgga gaggggccgg gagcgtgcag acaccaggcg |
| 241 | gtggcgcttt gacgcgactt tggagatcat cgtggtcatg aactccccca gcaatgacct |
| 301 | caacgacagc catgttcaga tttcctacca ctccagccat gagcctctgc ccctggccta |
| 361 | tgcggtgctc tacctcacct gtgttgacat ctctctggat tgcgacctga actgtgaggg |
| 421 | aaggcaggac aggaactttg tagacaagcg gcagtgggtc tgggggccca gtgggtatgg |
| 481 | cggcatcttg ctggtgaact gtgaccgtga tgatccgagc tgtgatgtcc aggacaattg |
| 541 | tgaccagcac gtgcactgcc tgcaagacct ggaagacatg tctgtcatgg tcctgcggac |
| 601 | gcagggccct gcagccctct ttgatgacca caaacttgtc ctccatacct ccagctatga |
| 661 | tgccaaacgg gcacaggtct tccacatctg cggtcctgag gatgtgtgtg aggcctatag |
| 721 | gcatgtgctg ggccaagata aggtgtccta tgaggtaccc cgcttgcatg gggatgagga |
| 781 | gcgcttcttc gtggaaggcc tgtccttccc tgatgccggc ttcacaggac tcatctcctt |
| 841 | ccatgtcact ctgctggacg actccaacga ggatttctcg gcatccccta tcttcactga |
| 901 | cactgtggtg ttccgagtgg caccctggat catgacgccc agcactctgc cacccctaga |
| 961 | ggtgtatgtg tgccgtgtga ggaacaacac gtgttttgtg gatgcggtgg cagagctggc |
| 1021 | caggaaggcc ggctgcaagc tgaccatctg cccacaggcc gagaaccgca acgaccgctg |
| 1081 | gatccaggat gagatggagc tgggctacgt tcaggcgccg cacaagaccc tcccggtggt |
| 1141 | ctttgactcc ccaaggaatg gggaactgca ggatttccct tacaaaagaa tcctgggtcc |
| 1201 | agattttggt tacgtgactc gggaaccacg cgacaggtct gtgagtggcc tggactcctt |
| 1261 | tgggaacctg gaggtcagcc ctccagtggt ggccaatggg aaagagtacc ccctggggag |
| 1321 | gatcctcatt gggggcaacc tgcctgggtc aagtggccgc agggtcaccc aggtggtgcg |
| 1381 | ggacttcctc catgcccaga aggtgcagcc ccccgtggag ctctttgtgg actggttggc |
| 1441 | cgtgggccat gtggatgagt ttctgagctt tgtccctgcc cccgatggga agggcttccg |
| 1501 | gatgctcctg gccagccctg gggcctgctt caagctcttc caggaaaagc agaagtgtgg |
| 1561 | ccacgggagg gccctcctgt tccagggggt tgttgatgat gagcaggtca agaccatctc |
| 1621 | catcaaccag gtgctctcca ataaagacct catcaactac aataagtttg tgcagagctg |
| 1681 | catcgactgg aaccgtgagg tgctgaagcg ggagctgggc ctggcagagt gtgacatcat |
| 1741 | tgacatccca cagctcttca agaccgagag gaaaaaagca acggccttct tccctgactt |
| 1801 | ggtgaacatg ctggtgctgg ggaagcacct gggcatcccc aagccctttg ggcccatcat |
| 1861 | caatggctgc tgctgcctgg aggagaaggt gcggtccctg ctggagccgc tgggcctcca |
| 1921 | ctgcaccttc attgatgact tcactccata ccacatgctg catggggagg tgcactgtgg |
| 1981 | caccaatgtg tgcagaaagc ccttctcttt caagtggtgg aacatggtgc cctgagacag |
| 2041 | ctcccaccca ccatcctgtc cccctggggc gggcattggc ccaggtggtg gagacagaga |
| 2101 | caggcccctg aacgataagc accaagagac cccaaggctc cagatggaac actgagggtg |
| 2161 | accgtccctc tcagaagcct tttccctgga agtgtccatg cctcacctgc aacccatgtg |
| 2221 | gttctcagac ttgaatcttc tcggcccccc aaaaagaagg acctcatttc ttatagcctc |
| 2281 | tcctgtgatt caacacaacc catggagatg tccccttctc actctgaaat catccatttg |
| 2341 | gggacaaatc cacattgggg tctagaaaca tccacgtatc tcatcagcca tcttgtcctg |
| 2401 | tgcatcctaa cagaggaagg atccatgatt ctgctttggt ccaattgctt cctctctgca |
| 2461 | gaggaacaac cctaaaacca gaccactcca cgcaggacag gcaggagaga ttcttcctaa |
| 2521 | agcctccccc ataaaaaggg agctgtggat ccacttagat cagggcggaa ccatattca |
| 2581 | cccggccaag ctcctgccca gatgttgacc ctcacccagc gtgagctgtc acatagtagg |
| 2641 | agcttctaga tgcatgtgga agcaatgaga gttgtccctt agccttataa actccccatg |
| 2701 | atctgacatg cagaaatcca gccttgtcca gaatcctcct ggaatttctt ggagacgaaa |
| 2761 | gtatctgggg gattgttggg tactagggag actgggtaca agggtgaaaa gtagttccca |
| 2821 | taatacacat ggttgactat ggtgatccac cttgtgatgg ttaatattag gtgtctggag |
| 2881 | aaggttgctt cattggccct gggacttctc tctgcaggag gagagaacgc tgcctctcct |
| 2941 | ctggattggt ctcaggctct ctgttggcct ttggtcagcg tttccacatc ctgctctgct |
| 3001 | gcaggagagg gggctaaggg gctggatcca ccaaggcagc tcacagcggg aaaactctgg |
| 3061 | gaatgaacca ctgaattcag gggatggggg tgggggggcg gttctcgagg tgtgtgccag |
| 3121 | ctacacgtgt gttctgtatg ggtccagctg cgtttccatc actcgctaat aaatcaacag |
| 3181 | aaacacaaa |

The atg start and stop codons are bolded and underlined. The amino acid sequence of human PADI3 (PAD3), provided by Genbank Accession No. NP_057317.2, is incorporated herein by reference, and is shown below (SEQ ID NO: 208).

| | |
|---|---|
| 1 | mslqrivrvs lehptsavcv agvetivdiy gsvpegtemf evygtpgvdi yispnmergr |
| 61 | eradtrrwrf datleiivvm nspsndlnds hvqisyhssh eplplayavl yltcvdisld |
| 121 | cdlncegrqd rnfvdkrqwv wgpsgyggil lvncdrddps cdvqdncdqh vhclqdledm |
| 181 | svmvlrtqgp aalfddhklv lhtssydakr aqvfhicgpe dvceayrhvl gqdkvsyevp |
| 241 | rlhgdeerff veglsfpdag ftglisfhvt llddsnedfs aspiftdtvv frvapwimtp |
| 301 | stlpplevyv crvrnntcfv davaelarka gcklticpqa enrndrwiqd emelgyvqap |
| 361 | hktlpvvfds prngelqdfp ykrilgpdfg yvtreprdrs vsgldsfgnl evsppvvang |
| 421 | keyplgrili ggnlpgssgr rvtqvvrdfl haqkvqppve lfvdwlavgh vdeflsfvpa |
| 481 | pdgkgfrmll aspgacfklf qekqkcghgr allfqgvvdd eqvktisinq vlsnkdliny |
| 541 | nkivqscidw nrevlkrelg laecdiidip qlfkterkka taffpdlvnm lvlgkhlgip |
| 601 | kpfgpiingc ccleekvrsl leplglhctf iddftpyhml hgevhcgtnv crkpfsfkww |
| 661 | nmvp |

The mRNA sequence encoding human FOXP3 provided by Genbank Accession No. EF534714.1, is incorporated herein by reference, and is shown below (SEQ ID NO: 209).

| | |
|---|---|
| 1 | atgcccaacc ccaggcctgg caagccctcg gccccttcct tggcccttgg cccatcccca |
| 61 | ggagcctcgc ccagctggag ggctgcaccc aaagcctcag acctgctggg ggcccggggc |
| 121 | ccagggggaa ccttccaggg ccgagatctt cgaggcgggg cccatgcctc ctcttcttcc |
| 181 | ttgaacccca tgccaccatc gcagctgcag ctgcccacac tgcccctagt catggtggca |
| 241 | ccctccgggg cacggctggg ccccttgccc cacttacagg cactcctcca ggacaggcca |
| 301 | catttcatgc accagctctc aacggtggat gcccacgccc ggacccctgt gctgcaggtg |
| 361 | caccccctgg agagcccagc catgatcagc ctcacaccac ccaccaccgc cactggggtc |
| 421 | ttctccctca aggcccggcc tggcctccca cctgggatca acgtggccag cctggaatgg |
| 481 | gtgtccaggg agccggcact gctctgcacc ttcccaaatc ccagtgcacc caggaaggac |
| 541 | agcacccttt cggctgtgcc ccagagctcc tacccactgc tggcaaatgg tgtctgcaag |
| 601 | tggcccggat gtgagaaggt cttcgaagag ccagaggact tcctcaagca ctgccaggcg |
| 661 | gaccatcttc tggatgagaa gggcagggca caatgtctcc tccagagaga gatggtacag |
| 721 | tctctggagc agcagctggt gctggagaag gagaagctga gtgccatgca ggcccacctg |
| 781 | gctgggaaaa tggcactgac caaggcttca tctgtggcat catccgacaa gggctcctgc |
| 841 | tgcatcgtag ctgctggcag ccaaggccct gtcgtcccag cctggtctgg cccccgggag |
| 901 | gcccctgaca gcctgtttgc tgtccggagg cacctgtggg gtagccatgg aaacagcaca |
| 961 | ttcccagagt tcctccacaa catggactac ttcaagttcc acaacatgcg accccctttc |
| 1021 | acctacgcca cgctcatccg ctgggccatc ctggaggctc cagagaagca gcggacactc |
| 1081 | aatgagatct accactggtt cacacgcatg tttgccttct tcagaaacca tcctgccacc |
| 1141 | tggaagaacg ccatccgcca caacctgagt ctgcacaagt gctttgtgcg ggtggagagc |
| 1201 | gagaaggggg ctgtgtggac cgtggatgag ctggagttcc gcaagaaacg gagccagagg |
| 1261 | cccagcaggt gttccaaccc tacacctggc ccctga |

The atg start and stop codons are bolded and underlined. The amino acid sequence of human FOXP3, provided by Genbank Accession No. ABQ15210.1, is incorporated herein by reference, and is shown below (SEQ ID NO: 210).

| | |
|---|---|
| 1 | mpnprpgkps apslalgpsp gaspswraap kasdllgarg pggtfqgrdl rggahassss |
| 61 | lnpmppsqlq lptlplvmva psgarlgplp hlqallqdrp hfmhqlstvd ahartpvlqv |
| 121 | hplespamis ltppttatgv fslkarpglp pginvaslew vsrepallct fpnpsaprkd |
| 181 | stlsavpqss ypllangvck wpgcekvfee pedflkhcqa dhlldekgra qcllqremvq |
| 241 | sleqqlvlek eklsamqahl agkmaltkas svassdkgsc civaagsqgp vvpawsgpre |
| 301 | apdslfavrr hlwgshgnst fpeflhnmdy flahnmrppf tyatlirwai leapekqrtl |
| 361 | neiyhwftrm faffrnhpat wknairhnls lhkcfvrves ekgavwtvde lefrkkrsqr |
| 421 | psrcsnptpg p |

The mRNA sequence encoding human IL2RA (CD-25) provided by Genbank Accession No. NM_000417.2, is incorporated herein by reference, and is shown below (SEQ ID NO: 211).

| (SEQ ID NO: 211) | |
|---|---|
| 1 | ggcagtttcc tggctgaaca cgccagccca atacttaaag agagcaactc ctgactccga |
| 61 | tagagactgg atggacccac aagggtgaca gcccaggcgg accgatcttc ccatcccaca |
| 121 | tcctccggcg cgatgccaaa aagaggctga cggcaactgg gccttctgca gagaaagacc |
| 181 | tccgcttcac tgccccggct ggtcccaagg gtcaggaaga tggattcata cctgctgatg |
| 241 | tggggactgc tcacgttcat catggtgcct ggctgccagg cagagctctg tgacgatgac |
| 301 | ccgccagaga tcccacacgc cacattcaaa gccatggcct acaaggaagg aaccatgttg |
| 361 | aactgtgaat gcaagagagg tttccgcaga ataaaaagcg ggtcactcta tatgctctgt |
| 421 | acaggaaact ctagccactc gtcctgggac aaccaatgtc aatgcacaag ctctgccact |
| 481 | cggaacacaa cgaaacaagt gacacctcaa cctgaagaac agaaagaaag gaaaaccaca |
| 541 | gaaatgcaaa gtccaatgca gccagtggac caagcgagcc ttccaggtca ctgcagggaa |
| 601 | cctccaccat gggaaaatga agccacagag agaatttatc atttcgtggt ggggcagatg |
| 661 | gtttattatc agtgcgtcca gggatacagg gctctacaca gaggtcctgc tgagagcgtc |
| 721 | tgcaaaatga cccacgggaa gacaaggtgg acccagcccc agctcatatg cacaggtgaa |
| 781 | atggagacca gtcagtttcc aggtgaagag aagcctcagg caagccccga aggccgtcct |
| 841 | gagagtgaga cttcctgcct cgtcacaaca acagattttc aaatacagac agaaatggct |
| 901 | gcaaccatgg agacgtccat atttacaaca gagtaccagg tagcagtggc cggctgtgtt |
| 961 | ttcctgctga tcagcgtcct cctcctgagt gggctcacct ggcagcggag acagaggaag |
| 1021 | agtagaagaa caatctagaa aaccaaaaga acaagaattt cttggtaaga agccgggaac |
| 1081 | agacaacaga agtcatgaag cccaagtgaa atcaaaggtg ctaaatggtc gcccaggaga |
| 1141 | catccgttgt gcttgcctgc gttttggaag ctctgaagtc acatcacagg acacggggca |
| 1201 | gtggcaacct tgtctctatg ccagctcagt cccatcagag agcgagcgct acccacttct |
| 1261 | aaatagcaat ttcgccgttg aagaggaagg gcaaaaccac tagaactctc catcttattt |
| 1321 | tcatgtatat gtgttcatta aagcatgaat ggtatggaac tctctccacc ctatatgtag |
| 1381 | tataaagaaa agtaggttta cattcatctc attccaactt cccagttcag gagtcccaag |
| 1441 | gaaagcccca gcactaacgt aaatacacaa cacacacact ctaccctata caactggaca |
| 1501 | ttgtctgcgt ggttcctttc tcagccgctt ctgactgctg attctcccgt tcacgttgcc |
| 1561 | taataaacat ccttcaagaa ctctgggctg ctacccagaa atcattttac ccttggctca |
| 1621 | atcctctaag ctaaccccct tctactgagc cttcagtctt gaatttctaa aaaacagagg |
| 1681 | ccatggcaga ataatctttg ggtaacttca aaacggggca gccaaaccca tgaggcaatg |
| 1741 | tcaggaacag aaggatgaat gaggtcccag gcagagaatc atacttagca aagttttacc |
| 1801 | tgtgcgttac taattggcct ctttaagagt tagtttcttt gggattgcta tgaatgatac |
| 1861 | cctgaatttg gcctgcacta atttgatgtt tacaggtgga cacacaaggt gcaaatcaat |
| 1921 | gcgtacgttt cctgagaagt gtctaaaaac accaaaaagg gatccgtaca ttcaatgttt |
| 1981 | atgcaaggaa ggaaagaaag aaggaagtga agagggagaa gggatggagg tcacactggt |
| 2041 | agaacgtaac cacggaaaag agcgcatcag gcctggcacg gtggctcagg cctataaccc |
| 2101 | cagctcccta ggagaccaag gcgggagcat ctcttgaggc caggagtttg agaccagcct |
| 2161 | gggcagcata gcaagacaca tccctacaaa aaattagaaa ttggctggat gtggtggcat |
| 2221 | acgcctgtag tcctagccac tcaggaggct gaggcaggag gattgcttga gcccaggagt |
| 2281 | tcgaggctgc agtcagtcat gatggcacca ctgcactcca gcctgggcaa cagagcaaga |
| 2341 | tcctgtcttt aaggaaaaaa agacaagatg agcataccag cagtccttga acattatcaa |
| 2401 | aaagttcagc atattagaat caccgggagg ccttgttaaa agagttcgct gggcccatct |
| 2461 | tcagagtctc tgagttgttg gtctggaata gagccaaatg ttttgtgtgt ctaacaattc |
| 2521 | ccaggtgctg ttgctgctgc tactattcca ggaacacact ttgagaacca ttgtgttatt |
| 2581 | gctctgcacg cccacccact ctcaactccc acgaaaaaaa tcaacttcca gagctaagat |
| 2641 | ttcggtggaa gtcctggttc catatctggt gcaagatctc ccctcacgaa tcagttgagt |
| 2701 | caacattcta gctcaacaac atcacacgat taacattaac gaaaattatt catttgggaa |
| 2761 | actatcagcc agttttcact tctgaagggg caggagagtg ttatgagaaa tcacggcagt |
| 2821 | tttcagcagg gtccagattc agattaaata actattttct gtcatttctg tgaccaacca |
| 2881 | catacaaaca gactcatctg tgcactctcc ccctccccct tcaggtatat gttttctgag |
| 2941 | taaagttgaa aagaatctca gaccagaaaa tatagatata tatttaaatc ttacttgagt |
| 3001 | agaactgatt acgacttttg ggtgttgagg ggtctataag atcaaaactt ttccatgata |
| 3061 | atactaagat gttatcgacc atttatctgt ccttctctca aaagtgtatg gtggaatttt |
| 3121 | ccagaagcta tgtgatacgt gatgatgtca tcactctgct gttaacatat aataaattta |
| 3181 | ttgctattgt ttataaaaga ataaatgata tttttt |

The atg start and stop codons are bolded and underlined. The amino acid sequence of human IL2RA (CD-25), provided by Genbank Accession No. NP_000408, is incorporated herein by reference, and is shown below (SEQ ID NO: 212).

| (SEQ ID NO: 212) | |
|---|---|
| 1 | mdsyllmwgl ltfimvpgcq aelcdddppe iphatfkama ykegtmlnce ckrgfrriks |
| 61 | gslymictgn sshsswdnqc qctssatrnt tkqvtpqpee qkerkttemq spmqpvdqas |
| 121 | lpghcreppp weneateriy hfvvgqmvyy qcvqgyralh rgpaesvckm thgktrwtqp |
| 181 | qlictgemet sqfpgeekpq aspegrpese tsclvtttdf qiqtemaatm etsiftteyq |
| 241 | vavagcvfll isvillsglt wqrrqrksrr ti |

(Signal protein AA 1-21; mature protein AA 22-272).

The mRNA sequence encoding human FAP (fibroblast activation protein) provided by Genbank Accession No. NM_001291807.1, is incorporated herein by reference, and is shown below (SEQ ID NO: 213).

| | |
|---|---|
| 1 | aagaacgccc ccaaaatctg tttctaattt tacagaaatc ttttgaaact tggcacggta |
| 61 | ttcaaaagtc cgtggaaaga aaaaaacctt gtcctggctt cagcttccaa ctacaaagac |
| 121 | agacttggtc cttttcaacg gttttcacag atccagtgac ccacgctctg aagacagaat |
| 181 | tagctaactt tcaaaaacat ctggaaaaat gaagacttgg gtaaaaatcg tatttggagt |
| 241 | tgccacctct gctgtgcttg ccttattggt gatgtgcatt gtcttacgcc cttcaagagt |
| 301 | tcataactct gaagaaaata caatgagagc actcacactg aaggatattt taaatggaac |
| 361 | attttcttat aaaacatttt ttccaaactg gatttcagga caagaatatc ttcatcaatc |
| 421 | tgcagataac aatatagtac tttataatat tgaaacagga caatcatata ccattttgag |
| 481 | taatagaacc atgctttgga gatactctta cacagcaaca tattacatct atgaccttag |
| 541 | caatggagaa tttgtaagag gaaatgagct tcctcgtcca attcagtatt tatgctggtc |
| 601 | gcctgttggg agtaaattag catatgtcta tcaaaacaat atctatttga aacaaagacc |
| 661 | aggagatcca ccttttcaaa taacatttaa tggaagagaa aataaaatat ttaatggaat |
| 721 | cccagactgg gtttatgaag aggaaatgct tgctacaaaa tatgctctct ggtggtctcc |
| 781 | taatggaaaa tttttggcat atgcggaatt taatgatacg gatataccag ttattgccta |
| 841 | ttcctattat ggcgatgaac aatatcctag aacaataaat attccatacc caaaggctgg |
| 901 | agctaagaat cccgttgttc ggatatttat tatcgatacc acttaccctg cgtatgtagg |
| 961 | tccccaggaa gtgcctgttc cagcaatgat agcctcaagt gattattatt tcagttggct |
| 1021 | cacgtgggtt actgatgaac gagtatgttt gcagtggcta aaaagagtcc agaatgtttc |
| 1081 | ggtcctgtct atatgtgact tcagggaaga ctggcagaca tgggattgtc caaagaccca |
| 1141 | ggagcatata gaagaaagca gaactggatg ggctggtgga ttctttgttt caacaccagt |
| 1201 | tttcagctat gatgccattt cgtactacaa aatatttagt gacaaggatg gctacaaaca |
| 1261 | tattcactat atcaaagaca ctgtggaaaa tgctattcaa attacaagtg gcaagtggga |
| 1321 | ggccataaat atattcagag taacacagga ttcactgttt tattctagca atgaatttga |
| 1381 | agaataccct ggaagaagaa acatctacag aattagcatt ggaagctatc ctccaagcaa |
| 1441 | gaagtgtgtt acttgccatc taaggaaaga aaggtgccaa tattacacag caagtttcag |
| 1501 | cgactacgcc aagtactatg cacttgtctg ctacggccca ggcatcccca tttccaccct |
| 1561 | tcatgatgga cgcactgatc aagaaattaa aatcctggaa gaaaacaagg aattggaaaa |
| 1621 | tgctttgaaa aatatccagc tgcctaaaga ggaaattaag aaacttgaag tagatgaaat |
| 1681 | tactttatgg tacaagatga ttcttcctcc tcaatttgac agatcaaaga agtatccctt |
| 1741 | gctaattcaa gtgtatggtg gtccctgcag tcagagtgta aggtctgtat ttgctgttaa |
| 1801 | ttggatatct tatcttgcaa gtaaggaagg gatggtcatt gccttggtgg atggtcgagg |
| 1861 | aacagctttc caaggtgaca aactcctcta tgcagtgtat cgaaagctgg gtgtttatga |
| 1921 | agttgaagac cagattacag ctgtcagaaa attcatagaa atgggtttca ttgatgaaaa |
| 1981 | aagaatagcc atatggggct ggtcctatgg aggatacgtt tcatcactgg cccttgcatc |
| 2041 | tggaactggt cttttcaaat gtggtatagc agtggctcca gtctccagct gggaatatta |
| 2101 | cgcgtctgtc tacacagaga gattcatggg tctcccaaca aaggatgata atcttgagca |
| 2161 | ctataagaat tcaactgtga tggcaagagc agaatatttc agaaatgtag actatcttct |
| 2221 | catccacgga acagcagatg ataatgtgca ctttcaaaac tcagcacaga ttgctaaagc |
| 2281 | tctggttaat gcacaagtgg atttccaggc aatgtggtac tctgaccaga accacggctt |
| 2341 | atccggcctg tccacgaacc acttatacac ccacatgacc cacttcctaa agcagtgttt |
| 2401 | ctctttgtca gactaaaaac gatgcagatg caagcctgta tcagaatctg aaaaccttat |
| 2461 | ataaacccct cagacagttt gcttatttta ttttttatgt tgtaaaatgc tagtataaac |
| 2521 | aaacaaatta atgttgttct aaaggctgtt aaaaaaaaga tgaggactca gaagttcaag |
| 2581 | ctaaatattg tttacatttt ctggtactct gtgaaagaag agaaaaggga gtcatgcatt |
| 2641 | ttgctttgga cacagtgttt tatcacctgt tcatttgaag aaaaataata aagtcagaag |
| 2701 | ttcaagtgct aaaaaaaaaa aaaaaaaaaa aaaaaaaaaa |

The atg start and stop codons are bolded and underlined. The amino acid sequence of human FAP (fibroblast activation protein), provided by Genbank Accession No. NP_001278736.1, is incorporated herein by reference, and is shown below (SEQ ID NO: 214).

| | |
|---|---|
| 1 | mktwvkivfg vatsavlall vmcivhpsr vhnseentmr altlkdilng tfsyktffpn |
| 61 | wisgqeylhq sadnnivlyn ietgqsytil snrtmlwrys ytatyyiydl sngefvrgne |
| 121 | lprpiqylcw spvgsklayv yqnniylkqr pgdppfqitf ngrenkifng ipdwvyeeem |
| 181 | latkyalwws pngkflayae fndtdipvia ysyygdeqyp rtinipypka gaknpvvrif |
| 241 | iidttypayv gpqevpvpam iassdyyfsw ltwvtdervc lqwlkrvqnv svlsicdfre |
| 301 | dwqtwdcpkt qehieesrtg waggffvstp vfsydaisyy kifsdkdgyk hihyikdtve |
| 361 | naiqitsgkw eainifrvtq dslfyssnef eeypgrrniy risigsypps kkcvtchlrk |
| 421 | ercqyytasf sdyakyyalv cygpgipist lhdgrtdqei kileenkele nalkniqlpk |
| 481 | eeikklevde itlwykmilp pqfdrskkyp lliqvyggpc sqsvrsvfav nwisylaske |
| 541 | gmvialvdgr gtafqgdkll yavyrklgvy evedqitavr kfiemgfide kriaiwgwsy |
| 601 | ggyvsslala sgtglfkcgi avapvsswey yasvyterfm glptkddnle hyknstvmar |
| 661 | aeyfrnvdyl lffigtaddnv hfqnsaqiak alvnaqvdfq amwysdqnhg lsglstnhly |
| 721 | thmthflkqc fslsd |

The mRNA sequence encoding human DPP4 (dipeptidyl peptidase 4) provided by Genbank Accession No. NM_001935.3, is incorporated herein by reference, and is shown below (SEQ ID NO: 215).

| | |
|---|---|
| 1 | ctttcactgg caagagacgg agtcctgggt ttcagttcca gttgcctgcg gtgggctgtg |
| 61 | tgagtttgcc aaagtcccct gccctctctg ggtctcggtt ccctcgcctg tccacgtgag |
| 121 | gttggaggag ctgaacgccg acgtcatttt tagctaagag ggagcagggt ccccgagtcg |
| 181 | ccggcccagg gtctgcgcat ccgaggccgc gcgccctttc ccctccccca cggctcctcc |
| 241 | gggccccgca ctctgcgccc cggctgccgc ccagcgccct acaccgccct cagggggccc |
| 301 | tcgcgggctc cccccggccg ggatgccagt gccccgcgcc acgcgcgcct gctcccgcgc |
| 361 | cgcctgccct gcagcctgcc cgcggcgcct ttatacccag cgggctcggc gctcactaat |
| 421 | gtttaactcg gggccgaaac ttgccagcgg cgagtgactc caccgcccgg agcagcggtg |
| 481 | caggacgcgc gtctccgccg cccgcggtga cttctgcctg cgctccttct ctgaacgctc |
| 541 | acttccgagg agacgccgac gatgaagaca ccgtggaagg ttcttctggg actgctgggt |
| 601 | gctgctgcgc ttgtcaccat catcaccgtg cccgtggttc tgctgaacaa aggcacagat |
| 661 | gatgctacag ctgacagtcg caaaacttac actctaactg attacttaaa aaatacttat |
| 721 | agactgaagt tatactcctt aagatggatt tcagatcatg aatatctcta caaacaagaa |
| 781 | aataatatct tggtattcaa tgctgaatat ggaaacagct cagttttctt ggagaacagt |
| 841 | acatttgatg agtttggaca ttctatcaat gattattcaa tatctcctga tgggcagttt |
| 901 | attctcttag aatacaacta cgtgaagcaa tggaggcatt cctacacagc ttcatatgac |
| 961 | atttatgatt taaataaaag gcagctgatt acagaagaga ggattccaaa caacacacag |
| 1021 | tgggtcacat ggtcaccagt gggtcataaa ttggcatatg tttggaacaa tgacatttat |
| 1081 | gttaaaattg aaccaaattt accaagttac agaatcacat ggacggggaa agaagatata |
| 1141 | atatataatg gaataactga ctgggtttat gaagaggaag tcttcagtgc ctactctgct |
| 1201 | ctgtggtggt ctccaaacgg cactttttta gcatatgccc aatttaacga cacagaagtc |
| 1261 | ccacttattg aatactcctt ctactctgat gagtcactgc agtacccaaa gactgtacgg |
| 1321 | gttccatatc caaaggcagg agctgtgaat ccaactgtaa agttctttgt tgtaaataca |
| 1381 | gactctctca gctcagtcac caatgcaact tccatacaaa tcactgctcc tgcttctatg |
| 1441 | ttgatagggg atcactactt gtgtgatgtg acatgggcaa cacaagaaag aatttctttg |
| 1501 | cagtggctca ggaggattca gaactattcg gtcatggata tttgtgacta tgatgaatcc |
| 1561 | agtggaagat ggaactgctt agtggcacgg caacacattg aaatgagtac tactggctgg |
| 1621 | gttggaagat ttaggccttc agaacctcat tttacccttg atggtaatag cttctacaag |
| 1681 | atcatcagca atgaagaagg ttacagacac atttgctatt tccaaataga taaaaaagac |
| 1741 | tgcacattta ttacaaaagg cacctgggaa gtcatcggga tagaagctct aaccagtgat |
| 1801 | tatctatact acattagtaa tgaatataaa ggaatgccag gaggaaggaa tctttataaa |
| 1861 | atccaactta gtgactatac aaaagtgaca tgcctcagtt gtgagctgaa tccggaaagg |
| 1921 | tgtcagtact attctgtgtc attcagtaaa gaggcgaagt attatcagct gagatgttcc |
| 1981 | ggtcctggtc tgcccctcta tactctacac agcagcgtga atgataaagg gctgagagtc |
| 2041 | ctggaagaca attcagcttt ggataaaatg ctgcagaatg tccagatgcc ctccaaaaaa |
| 2101 | ctggacttca ttattttgaa tgaaacaaaa ttttggtatc agatgatctt gcctcctcat |
| 2161 | tttgataaat ccaagaaata tcctctacta ttagatgtgt atgcaggccc atgtagtcaa |
| 2221 | aaagcagaca ctgtcttcag actgaactgg gccacttacc ttgcaagcac agaaaacatt |
| 2281 | atagtagcta gctttgatgg cagaggaagt ggttaccaag gagataagat catgcatgca |
| 2341 | atcaacagaa gactgggaac atttgaagtt gaagatcaaa ttgaagcagc cagacaattt |
| 2401 | tcaaaaatgg gatttgtgga caacaaacga attgcaattt ggggctggtc atatggaggg |
| 2461 | tacgtaacct caatggtcct gggatcggga agtggcgtgt tcaagtgtgg aatagccgtg |
| 2521 | gcgcctgtat cccggtggga gtactatgac tcagtgtaca cagaacgtta catgggtctc |
| 2581 | ccaactccag aagacaacct tgaccattac agaaattcaa cagtcatgag cagagctgaa |
| 2641 | aattttaaac aagttgagta cctccttatt catggaacag cagatgataa cgttcacttt |
| 2701 | cagcagtcag ctcagatctc caaagccctg gtcgatgttg gagtggattt ccaggcaatg |
| 2761 | tggtatactg atgaagacca tggaatagct agcagcacag cacaccaaca tatatatacc |
| 2821 | cacatgagcc acttcataaa acaatgtttc tctttacctt agcacctcaa aataccatgc |
| 2881 | catttaaagc ttattaaaac tcatttttgt tttcattatc tcaaaactgc actgtcaaga |
| 2941 | tgatgatgat ctttaaaata cacactcaaa tcaagaaact taaggttacc tttgttccca |
| 3001 | aatttcatac ctatcatctt aagtagggac ttctgtcttc acaacagatt attaccttac |
| 3061 | agaagtttga attatccggt cgggttttat tgtttaaaat catttctgca tcagctgctg |
| 3121 | aaacaacaaa taggaattgt ttttatggag gctttgcata gattccctga gcaggatttt |
| 3181 | aatctttttc taactggact ggttcaaatg ttgttctctt ctttaaaggg atggcaagat |
| 3241 | gtgggcagtg atgtcactag ggcagggaca ggataagagg gattagggag agaagatagc |
| 3301 | agggcatggc tgggaaccca agtccaagca taccaacacg agcaggctac tgtcagctcc |
| 3361 | cctcggagaa gagctgttca cagccagact ggcacagttt tctgagaaag actattcaaa |
| 3421 | cagtctcagg aaatcaaata tgcaaagcac tgacttctaa gtaaaaccac agcagttgaa |
| 3481 | aagactccaa agaaatgtaa gggaaactgc cagcaacgca ggcccccagg tgccagttat |
| 3541 | ggctataggt gctacaaaaa cacagcaagg gtgatgggaa agcattgtaa atgtgctttt |
| 3601 | aaaaaaaaat actgatgttc ctagtgaaag aggcagcttg aaactgagat gtgaacacat |
| 3661 | cagcttgccc tgttaaaaga tgaaaatatt tgtatcacaa atcttaactt gaaggagtcc |
| 3721 | ttgcatcaat ttttcttatt tcatttcttt gagtgtctta attaaaagaa tattttaact |
| 3781 | tccttggact cattttaaaa aatggaacat aaaatacaat gttatgtatt attattccca |
| 3841 | ttctacatac tatggaattt ctcccagtca tttaataaat gtgccttcat tttttcagaa |
| 3901 | aaaaaaaaaa aaa |

The atg start and stop codons are bolded and underlined. The amino acid sequence of human DPP-4 (dipeptidyl peptidase 4), provided by Genbank Accession No. NP_001926.2, is incorporated herein by reference, and is shown below (SEQ ID NO: 216).

| | |
|---|---|
| 1 | mktpwkvllg llgaaalvti itvpvvllnk gtddatadsr ktythdylk ntyrlklysl |
| 61 | rwisdheyly kqennilvfn aeygnssvfl enstfdefgh sindysispd gqfilleyny |
| 121 | vkqwrhsyta sydiydlnkr qliteeripn ntqwvtwspv ghklayvwnn diyvkiepnl |
| 181 | psyritwtgk ediiyngitd wvyeeevfsa ysalwwspng tflayaqfnd tevplieysf |
| 241 | ysdeslqypk tvrvpypkag avnptvkffv vntdslssvt natsiqitap asmligdhyl |
| 301 | cdvtwatqer islqwlrriq nysvmdicdy dessgrwncl varqhiemst tgwvgrfrps |
| 361 | ephftldgns fykiisneeg yrhicyfqid kkdctfitkg twevigieal tsdylyyisn |
| 421 | eykgmpggrn lykiqlsdyt kvtclsceln percqyysvs fskeakyyql rcsgpglply |
| 481 | tlhssvndkg lrvlednsal dkmlqnvqmp skkldfiiln etkfwyqmil pphfdkskky |
| 541 | pllldvyagp csqkadtvfr lnwatylast eniivasfdg rgsgyqgdki mhainrrlgt |
| 601 | fevedqieaa rqfskmgfvd nkriaiwgws yggyvtsmvl gsgsgvfkcg iavapvsrwe |
| 661 | yydsvytery mglptpednl dhyrnstvms raenfkqvey llihgtaddn vhfqqsaqis |
| 721 | kalvdvgvdf qamwytdedh giasstahqh iythmshfik qcfslp |

The mRNA sequence encoding human CD26 provided by Genbank Accession No. M74777.1, is incorporated herein by reference, and is shown below (SEQ ID NO: 217).

| | |
|---|---|
| 1 | gacgccgacg atgaagacac cgtggaaggt tcttctggga ctgctgggtg ctgctgcgct |
| 61 | tgtcaccatc atcaccgtgc ccgtggttct gctgaacaaa ggcacagatg atgctacagc |
| 121 | tgacagtcgc aaaacttaca ctctaactga ttacttaaaa aatacttata gactgaagtt |
| 181 | atactcctta agatggattt cagatcatga atatctctac aaacaagaaa ataatatctt |
| 241 | ggtattcaat gctgaatatg gaaacagctc agttttcttg gagaacagta catttgatga |
| 301 | gtttggacat tctatcaatg attattcaat atctcctgat gggcagttta ttctcttaga |
| 361 | atacaactac gtgaagcaat ggaggcattc ctacacagct tcatatgaca tttatgattt |
| 421 | aaataaaagg cagctgatta cagaagagag gattccaaac aacacacagt gggtcacatg |
| 481 | gtcaccagtg ggtcataaat tggcatatgt ttggaacaat gacatttatg ttaaaattga |
| 541 | accaaattta ccaagttaca gaatcacatg gacggggaaa gaagatataa tatataatgg |
| 601 | aataactgac tgggtttatg aagaggaagt cttcagtgcc tactctgctc tgtggtggtc |
| 661 | tccaaacggc acttttttag catatgccca atttaacgac acagaagtcc cacttattga |
| 721 | atactccttc tactctgatg agtcactgca gtacccaaag actgtacggg ttccatatcc |
| 781 | aaaggcagga gctgtgaatc caactgtaaa gttctttgtt gtaaatacag actctctcag |
| 841 | ctcagtcacc aatgcaactt ccatacaaat cactgctcct gcttctatgt tgatagggga |
| 901 | tcactacttg tgtgatgtga catgggcaac acaagaaaga atttctttgc agtggctcag |
| 961 | gaggattcag aactattcgg tcatggatat ttgtgactat gatgaatcca gtggaagatg |
| 1021 | gaactgctta gtggcacggc aacacattga aatgagtact actggctggg ttggaagatt |
| 1081 | taggccttca gaacctcatt ttacccttga tggtaatagc ttctacaaga tcatcagcaa |
| 1141 | tgaagaaggt tacagacaca tttgctattt ccaaatagat aaaaaagact gcacatttat |
| 1201 | tacaaaaggc acctgggaag tcatcgggat agaagctcta accagtgatt atctatacta |
| 1261 | cattagtaat gaatataaag gaatgccagg aggaaggaat ctttataaaa tccaacttag |
| 1321 | tgactataca aaagtgacat gcctcagttg tgagctgaat ccggaaaggt gtcagtacta |
| 1381 | ttctgtgtca ttcagtaaag aggcgaagta ttatcagctg agatgttccg gtcctggtct |
| 1441 | gcccctctat actctacaca gcagcgtgaa tgataaaggg ctgagagtcc tggaagacaa |
| 1501 | ttcagctttg gataaaatgc tgcagaatgt ccagatgccc tccaaaaaac tggacttcat |
| 1561 | tattttgaat gaaacaaaat tttggtatca gatgatcttg cctcctcatt ttgataaatc |
| 1621 | caagaaatat cctctactat tagatgtgta tgcaggccca tgtagtcaaa aagcagacac |
| 1681 | tgtcttcaga ctgaactggg ccacttacct tgcaagcaca gaaaacatta tagtagctag |
| 1741 | ctttgatggc agaggaagtg gttaccaagg agataagatc atgcatgcaa tcaacagaag |
| 1801 | actgggaaca tttgaagttg aagatcaaat tgaagcagcc agacaatttt caaaaatggg |
| 1861 | atttgtggac aacaaacgaa ttgcaatttg gggctggtca tatggagggt acgtaacctc |
| 1921 | aatggtcctg ggatcaggaa gtggcgtgtt caagtgtgga atagccgtgg cgcctgtatc |
| 1981 | ccggtgggag tactatgact cagtgtacac agaacgttac atgggtctcc caactccaga |
| 2041 | agacaacctt gaccattaca gaaattcaac agtcatgagc agagctgaaa attttaaaca |
| 2101 | agttgagtac ctccttattc atggaacagc agatgataac gttcactttc agcagtcagc |
| 2161 | tcagatctcc aaagccctgg tcgatgttgg agtggatttc caggcaatgt ggtatactga |
| 2221 | tgaagaccat ggaatagcta gcagcacagc acaccaacat atatataccc acatgagcca |
| 2281 | cttcataaaa caatgtttct ctttacctta gcacctcaaa ataccatgcc atttaaagct |
| 2341 | tattaaaact catttttgtt ttcattatct caaaactgca ctgtcaagat gatgatgatc |
| 2401 | tttaaaatac acactcaaat caagaaactt aaggttacct ttgttcccaa atttcatacc |
| 2461 | tatcatctta agtagggact tctgtcttca caacagatta ttaccttaca gaagtttgaa |
| 2521 | ttatccggtc gggttttatt gtttaaaatc atttctgcat cagctgctga aacaacaaat |
| 2581 | aggaattgtt tttatggagg ctttgcatag attccctgag caggatttta atctttttct |
| 2641 | aactggactg gttcaaatgt tgttctcttc tttaaaggga tggcaagatg tgggcagtga |
| 2701 | tgtcactagg gcagggacag gataagaggg attagggaga gaagatagca gggcatggct |
| 2761 | gggaacccaa gtccaagcat accaacacga ccaggctact gtcagctccc ctcggagaaa |
| 2821 | actgtgcagt ctgcgtgtga acagctcttc tcctttagag cacaatggat ctcgagggat |
| 2881 | cttccatacc taccagttct gcgcctcgag gccgcgactc taga |

The atg start and stop codons are bolded and underlined. The amino acid sequence of human CD26, provided by Genbank Accession No. AAA51943.1, is incorporated herein by reference, and is shown below (SEQ ID NO: 218).

| | |
|---|---|
| 1 | mktpwkvllg llgaaalvti itvpvvllnk gtddatadsr ktytltdylk ntyrlklysl |
| 61 | rwisdheyly kqennilvfn aeygnssvfl enstfdefgh sindysispd gqfilleyny |
| 121 | vkqwrhsyta sydiydlnkr qliteeripn ntqwvtwspv ghklayvwnn diyvkiepnl |
| 181 | psyritwtgk ediiyngitd wvyeeevfsa ysalwwspng tflayaqfnd tevplieysf |
| 241 | ysdeslqypk tvrvpypkag avnptvkffv vntdslssvt natsiqitap asmligdhyl |
| 301 | cdvtwatqer islqwlrriq nysvmdicdy dessgrwncl varqhiemst tgwvgrfrps |
| 361 | ephftldgns fykiisneeg yrhicyfqid kkdctfitkg twevigieal tsdylyyisn |
| 421 | eykgmpggrn lykiqlsdyt kvtclsceln percqyysvs fskeakyyql rcsgpglply |
| 481 | tlhssvndkg lrvlednsal dkmlqnvqmp skkldfiiln etkfwyqmil pphfdkskky |
| 541 | pllldvyagp csqkadtvfr lnwatylast eniivasfdg rgsgyqgdki mhainttlgt |
| 601 | fevedqieaa rqfskmgfvd nkriaiwgws yggyvtsmvl gsgsgvfkcg iavapvsrwe |
| 661 | yydsvytery mglptpednl dhyrnstvms raenfkqvey llihgtaddn vhfqqsaqis |
| 721 | kalvdvgvdf qamwytdedh giasstahqh iythmshfik qcfslp |

The mRNA sequence encoding human SIRT1 provided by Genbank Accession No. JQ768366.1, is incorporated herein by reference, and is shown below (SEQ ID NO: 219).

| | |
|---|---|
| 1 | atgattggca cagatcctcg aacaattctt aaagatttat tgccggaaac aatacctcca |
| 61 | cctgagttgg atgatatgac actgtggcag attgttatta atatcctttc agaaccacca |
| 121 | aaaaggaaaa aaagaaaaga tattaataca attgaagatg ctgtgaaatt actgcaagag |
| 181 | tgcaaaaaaa ttatagttct aactggagct ggggtgtctg tttcatgtgg aatacctgac |
| 241 | ttcaggtcaa gggatggtat ttatgctcgc cttgctgtag acttcccaga tcttccagat |
| 301 | cctcaagcga tgtttgatat tgaatatttc agaaaagatc caagaccatt cttcaagttt |
| 361 | gcaaaggaaa tatatcctgg acaattccag ccatctctct gtcacaaatt catagccttg |
| 421 | tcagataagg aaggaaaact acttcgcaac tatacccaga acatagacac gctggaacag |
| 481 | gttgcgggaa tccaaaggat aattcagtgt catggttcct ttgcaacagc atcttgcctg |
| 541 | atttgtaaat acaaagttga ctgtgaagct gtacgaggag ctctttttag tcaggtagtt |
| 601 | cctcgatgtc ctaggtgccc agctgatgaa ccgcttgcta tcatgaaacc agagattgtg |
| 661 | ttttttggtg aaaatttacc agaacagttt catagagcca tgaagtatga caaagatgaa |
| 721 | gttgacctcc tcattgttat tgggtcttcc ctcaaagtaa gaccagtagc actaattcca |
| 781 | agttccatac cccatgaagt gcctcagata ttaattaata gagaaccttt gcctcatctg |
| 841 | cattttgatg tagagcttct tggagactgt gatgtcataa ttaatgaatt gtgtcatagg |
| 901 | ttaggtggtg aatatgccaa actttgctgt aaccctgtaa agctttcaga aattactgaa |
| 961 | aaacctccac gaacacaaaa agaattggct tatttgtcag agttgccacc cacacctctt |
| 1021 | catgtttcag aagactcaag ttcaccagaa agaacttcac caccagattc ttcagtgatt |
| 1081 | gtcacacttt tagaccaagc agctaagagt aatgatgatt tagatgtgtc tgaatcaaaa |
| 1141 | ggttgtatgg aagaaaaacc acaggaagta caaacttcta ggaatgttga aagtattgct |
| 1201 | gaacagatgg aaaatccgga tttgaagaat gttggttcta gtactgggga gaaaaatgaa |

The atg start and stop codons are bolded and underlined. The amino acid sequence of human SIRT1, provided by Genbank Accession No. JQ768366.1, is incorporated herein by reference, and is shown below (SEQ ID NO: 220).

| | |
|---|---|
| 1 | migtdprtil kdllpetipp pelddmtlwq ivinilsepp krkkrkdint iedavkllqe |
| 61 | ckkiivltga gvsvscgipd frsrdgiyar lavdfpdlpd pqamfdieyf rkdprpffkf |
| 121 | akeiypgqfq pslchkfial sdkegkllrn ytqnidtleq vagiqriiqc hgsfatascl |
| 181 | ickykvdcea vrgalfsqvv prcprcpade plaimkpeiv ffgenlpeqf hramkydkde |
| 241 | vdllivigss lkvrpvalip ssiphevpqi linreplphl hfdvellgdc dviinelchr |
| 301 | lggeyaklcc npvklseite kpprtqkela ylselpptpl hvsedssspe rtsppdssvi |
| 361 | vtlldqaaks nddldvsesk gcmeekpqev qtsrnvesia eqmenpdlkn vgsstgekne |

The mRNA sequence encoding human FoxO3a (forkhead box 03) provided by Genbank Accession No. NM_001455.3, is incorporated herein by reference, and is shown below (SEQ ID NO: 221).

| | |
|---|---|
| 1 | gcgcgaggcc gtcgattcgc tcgcggctcc atcgcggcct ggccgggggg cggtgtctgc |
| 61 | tgcgccaggt tcgctggccg cacgtcttca ggtcctcctg ttcctgggag gcgggcgcgg |
| 121 | caggactggg aggtggcggc agcgggcgag gactcgccga ggacggggct ccggcccggg |
| 181 | ataaccaact ctccttctct cttctttggt gcttccccag gcggcggcgg cggcgcccgg |
| 241 | gagccggagc cttcgcggcg tccacgtccc tcccccgctg caccccgccc cggcgcgaga |
| 301 | ggagagcgcg agagccccag ccgcgggcgg gcgggcggcg aagatggcag aggcaccggc |
| 361 | ttccccggcc ccgctctctc cgctcgaagt ggagctggac ccggagttcg agccccagag |
| 421 | ccgtccgcga tcctgtacgt ggcccctgca aaggccggag ctccaagcga gccctgccaa |
| 481 | gccctcgggg gagacggccg ccgactccat gatccccgag gaggaggacg atgaagacga |
| 541 | cgaggacggc gggggacggg ccggctcggc catggcgatc ggcggcggcg gcgggagcgg |
| 601 | cacgctgggc tccgggctgc tccttgagga ctcggcccgg gtgctggcac ccggagggca |
| 661 | agaccccggg tctgggccag ccaccgcggc gggcgggctg agcgggggta cacaggcgct |
| 721 | gctgcagcct cagcaaccgc tgccaccgcc gcagccgggg gcggctgggg gctccgggca |
| 781 | gccgaggaaa tgttcgtcgc ggcggaacgc ctggggaaac ctgtcctacg cggacctgat |
| 841 | cacccgcgcc atcgagagct ccccggacaa acggctcact ctgtcccaga tctacgagtg |
| 901 | gatggtgcgt tgcgtgccct acttcaagga taagggcgac agcaacagct ctgccggctg |
| 961 | gaagaactcc atccggcaca acctgtcact gcatagtcga ttcatgcggg tccagaatga |
| 1021 | gggaactggc aagagctctt ggtggatcat caaccctgat ggggggaaga gcggaaaagc |
| 1081 | cccccggcgg cgggctgtct ccatggacaa tagcaacaag tataccaaga gccgtggccg |
| 1141 | cgcagccaag aagaaggcag ccctgcagac agcccccgaa tcagctgacg acagtccctc |
| 1201 | ccagctctcc aagtggcctg gcagccccac gtcacgcagc agtgatgagc tggatgcgtg |
| 1261 | gacggacttc cgttcacgca ccaattctaa cgccagcaca gtcagtggcc gcctgtcgcc |
| 1321 | catcatggca agcacagagt tggatgaagt ccaggacgat gatgcgcctc tctcgcccat |
| 1381 | gctctacagc agctcagcca gcctgtcacc ttcagtaagc aagccgtgca cggtggaact |
| 1441 | gccacggctg actgatatgg caggcaccat gaatctgaat gatgggctga ctgaaaacct |
| 1501 | catggacgac ctgctggata acatcacgct cccgccatcc cagccatcgc ccactggggg |
| 1561 | actcatgcag cggagctcta gcttcccgta taccaccaag ggctcgggcc tgggctcccc |
| 1621 | aaccagctcc tttaacagca cggtgttcgg accttcatct ctgaactccc tacgccagtc |
| 1681 | tcccatgcag accatccaag agaacaagcc agctaccttc tcttccatgt cacactatgg |
| 1741 | taaccagaca ctccaggacc tgctcacttc ggactcactt agccacagcg atgtcatgat |
| 1801 | gacacagtcg gaccccttga tgtctcaggc cagcaccgct gtgtctgccc agaattcccg |
| 1861 | ccggaacgtg atgcttcgca atgatccgat gatgtccttt gctgcccagc ctaaccaggg |
| 1921 | aagtttggtc aatcagaact tgctccacca ccagcaccaa acccagggcg ctcttggtgg |
| 1981 | cagccgtgcc ttgtcgaatt ctgtcagcaa catgggcttg agtgagtcca gcagccttgg |
| 2041 | gtcagccaaa caccagcagc agtctcctgt cagccagtct atgcaaaccc tctcggactc |
| 2101 | tctctcaggc tcctccttgt actcaactag tgcaaacctg cccgtcatgg gccatgagaa |
| 2161 | gttccccagc gacttggacc tggacatgtt caatgggagc ttggaatgtg acatggagtc |
| 2221 | cattatccgt agtgaactca tggatgctga tgggttggat tttaactttg attccctcat |
| 2281 | ctccacacag aatgttgttg gtttgaacgt ggggaacttc actggtgcta agcaggcctc |
| 2341 | atctcagagc tgggtgccag gctgaaggat cactgaggaa ggggaagtgg gcaaagcaga |
| 2401 | ccctcaaact gacacaagac ctacagagaa aaccctttgc caaatctgct ctcagcaagt |
| 2461 | ggacagtgat accgtttaca gcttaacacc tttgtgaatc ccacgccatt ttcctaaccc |
| 2521 | agcagagact gttaatggcc ccttaccctg ggtgaagcac ttacccttgg aacagaactc |
| 2581 | taaaaagtat gcaaaatctt ccttgtacag ggtggtgagc cgcctgccag tggaggacag |
| 2641 | cacccctcag caccacccac cctcattcag agcacaccgt gagcccccgt cggccattct |
| 2701 | gtggtgtttt aatattgcga tggtttatgg gacgttttaa gtgttgttct tgtgtttgtt |
| 2761 | ttcctttgac tttctgagtt tttcacatgc attaacttgc ggtatttttc tgttaaaatg |
| 2821 | ttaaccgtcc ttcccctagc aaatttaaaa acagaaagaa aatgttgtac cagttaccat |
| 2881 | tccgggttcg agcatcacaa gcttttgagc gcatggaact ccataaacta acaaattaca |
| 2941 | taaactaaag ggggattttc tttcttcttt tgtttggtag aaaattatcc ttttctaaaa |
| 3001 | actgaacaat ggcacaattg tttgctatgt gcacccgtcc aggacagaac cgtgcatagg |
| 3061 | caaaaggagt ggagcacagc gtccggccca gtgtgtttcc ggttctgagt cagggtgatc |
| 3121 | tgtggacggg accccagcac caagtctacg ggtgccagat cagtagggcc tgtgatttcc |
| 3181 | tgtcagtgtc ctcagctaat gtgaacagtg ttggtctgct ggttagaaac tagaatattg |
| 3241 | atattttcag gaaagaaatc agctcagctc tccactcatt gccaaatgtc actaaagggt |
| 3301 | ttagttttaa ggagaaagaa aaggaaaaaa aaaaaaaaca aaaaagtcct gttttgcttt |
| 3361 | gcagaacaaa tgaacttaca ggtgagcatt aagcttgcag tgagaaatgt gcgaagagta |
| 3421 | aaaacccaag tcaatgctga ggcagttcta acttcactgt tttcctaaat acacatcctt |
| 3481 | gattattttc agccttgcta tataatctga tctgctagaa gtgtatgagt gagaggcaat |
| 3541 | agcatacaaa ctgatttttt aaatataagc ttaggttgta attgtacaag tgactcaatg |
| 3601 | gaagtacaaa atagggcagt tttaactttt ttttctgctt ctatggattt cattttgttg |
| 3661 | tgttttcaaa aagttatggt gctgtatagg tgctttctgt ttaacctgga aagtgtgatt |
| 3721 | atattcgtta ccttctttgg tagacggaat agttgggacc acctttggta cataagaaat |
| 3781 | tggtataacg atgctctgat tagcacagta tatgcatact tctccaaagt gatatatgaa |
| 3841 | gactcttttc tttgcataaa aagcattagg catataaatg tataaatata ttttatcatg |
| 3901 | tacagtacaa aaatggaacc ttatgcatgg gccttaggaa tacaggctag tatttcagca |
| 3961 | cagacttccc tgcttgagtt cttgctgatg cttgcaccgt gacagtgggc accaacacag |
| 4021 | acgtgccacc caaccccctg cacacaccac cggccaccag gggccccctt gtgcgccttg |
| 4081 | gctttataac tcctctgggg gtgatattgg tggtgatcac agctcctagc ataatgagag |
| 4141 | ttccatttgg tattgtcaca cgtctcctgc ctcgcttggg ttgccatgtt tgagcgatgg |
| 4201 | ccctgttgat ttcaccctgc cttttactga atctgtaaat tgttgtgcaa ttgtggttat |
| 4261 | agtagactgt agcacattgc cttttctaaa ctgctacatg tttataatct tcatttttaa |
| 4321 | agtatgtgta atttttttaa gtatgtattc tattcatatg gtctgcttgt cagtgagcca |
| 4381 | gacttgctta ctatattcct ttataataat gctagccact tcctggattc tttagtaatg |
| 4441 | tgctgtatgc aagaactttc cagtagcagt gaaggagggt tgcctctcca agcttcctaa |
| 4501 | gggatgctgc cctgtgtggg gatgcattgc agaggcacta gtagcatggg ggctagagtg |
| 4561 | gggagcgaga tgtaaaaggg tggggggata ggagaattcc agagtgcttc cagcattagg |
| 4621 | gtcctgagaa cttctgagtt cagagaaaca tgcaaagtga ctaacaaaat agctacttac |
| 4681 | ctttgcagtt ttacagaccc tgggagctgc tttgggagtg agaaaggcaa ccctccaatg |
| 4741 | tgtttcaact ttaaaatgtt gaattctttt cagacatggt atctcattta ttctcctttt |
| 4801 | ctagcgtttg ttgaatttca ggcagaatgt cttacagaat gtcctagaac cagattatca |
| 4861 | tttaatctga aacagctgag gaagggacag agaaggtaca agggcaaggc agcacaaaac |
| 4921 | agatcaggag aatgaagagg gaatgctttg gttttttgtt ttgttttgtt ttttcttttt |
| 4981 | caagtaacta aaacagcatc tacatgtaga gtgttgtgga gagctgagac cagggtaaag |
| 5041 | tcaagtgcag catcagtact gcgagaccca ccagcccctg gagagggtca gccgagaatc |
| 5101 | tggtagtgaa gcctgtctag ggtcccggca ccctcaccct cagccacctg cagagaggcc |
| 5161 | agggccccag agactagcct ggttctgaag tgggcagggg tgctgccaga gccctctgcc |
| 5221 | ccttatgttg agaccctgct ttcaggacag gccagccgtt ggccaccatg tcacattctg |
| 5281 | agtgagtgtc acaggtccct aacaataatt ttctgatctg gagcatatca gcagaatgct |
| 5341 | tagcctcaag gggcctggca gctgtaatgt ttgatttatg atgagaacta tccgaggcca |
| 5401 | cccttggcct ctaaataagc tgctctaggg agccgcctac tttttgatga gaaattagaa |
| 5461 | gagtacctaa tgttgaaaac atgacatgcg ctcttgggat ctgctgttct ctccagggct |
| 5521 | ccagaacctg atacctgtta ccaaagctag gaaagagctt tatcacaagc cttcactgtc |
| 5581 | ctggcatgag aactggctgc caggctcagt gtaccccatt aactgtgaat gaatctgagc |
| 5641 | ttggtttcct ttattgcttc ctctgcaata tgattgctga aacacatttt aaaaattcag |
| 5701 | aagcttgtca ctcctgttaa tgggaggatc agtcacacat gtgtagtaca aggcggactt |
| 5761 | tgtgtttgtt tttggtgtta atttttagca ttgtgtgtgt tgcttcccca ccctgaggag |
| 5821 | aggacaccat ggcttactac tcaggacaag tatgccccgc tcagggtgtg atttcaggtg |
| 5881 | gcttccaaac ttgtacgcag tttaaagatg gtggggacag actttgcctc tacctagtga |
| 5941 | accccactta aagaataagg agcatttgaa tctcttggaa aaggccatga agaataaagc |
| 6001 | agtcaaaaag aagtcctcca tgttggtgcc aaggacttgc gaggggaaat aaaaatgtta |
| 6061 | tccagcctga ccaacatgga gaaaccccgt ctccattaaa aatacaaaat tagcctggca |
| 6121 | tggtggcgca tgcctgtaat cccagctact ctggaggctg aggcaggaga atcgcttgaa |
| 6181 | cccaggaggc ggaggtcgca gtgagccgag atcatgccag tgcactccag cctgggtaac |
| 6241 | aagagtgaaa ctccgtgtca aaaaaaaaaa aaaaatgtta ctcatcctct ctgaaagcaa |
| 6301 | aaaggaaacc ctaacagctc tgaactctgg ttttattttt cttgctgtat ttgggtgaac |
| 6361 | attgtatgat taggcataat gttaaaaaaa aaaatttttt tttggtagaa atgcaatcac |
| 6421 | cagtaaagag gtacgaaaaa gctagcctct ctcagagacc ggggaggcag agtactacta |
| 6481 | gaggaagtga agttctgatg gaatcatgcc tgtcaaatga ggtcttgaag cggatgccca |
| 6541 | aataaaagag tatattttat ctaaatctta agtgggtaac attttatgca gtttaaatga |
| 6601 | atggaatatt ttcctcttgt ttagttgtat ctgtttgtat ttttctttga tgaatgattg |
| 6661 | gtcatgaggc ctcttgccac actccagaaa tacgtgtgcg gctgctttta agaactatgt |
| 6721 | gtctggtcac ttatttctct aaaattatct cattgcctgg caatcagtct tctcttgtat |
| 6781 | acttgtccta gcacattatg tacatgggaa atgtaaacaa atgtgaagga ggaccagaaa |
| 6841 | aattagttaa tatttaaaaa aatgtattgt gcattttggc ttcacatgtt taactttttt |
| 6901 | taagaaaaaa gttgcatgaa tggaaaaaaa aatctgtata cagtatctgt aaaaactatc |
| 6961 | ttatctgttt caattccttg ctcatatccc atataatcta gaactaaata tggtgtgtgg |
| 7021 | ccatatttaa acacctgaga gtcaagcagt tgagactttg atttgaagca cctcatcctt |
| 7081 | ctttcaatgc gaacactatc atatggcatt cttactgagg attttgtcta accatatgtt |
| 7141 | gccatgaatt aactctgccg cctttcttaa ggatcaaaac cagtttgatt tgggaatctt |
| 7201 | cccctttcca aatgaaatag agatgcagta cttaactttc cttggtgttt gtagatattg |
| 7261 | ccttgtgtat tccacttaaa accgtaatct agtttgtaaa agagatggtg acgcatgtaa |
| 7321 | ataaagcatc agtgacactc t |

The atg start and stop codons are bolded and underlined. The amino acid sequence of human FoxO3a (forkhead box 03), provided by Genbank Accession No. NP_001446.1, is incorporated herein by reference, and is shown below (SEQ ID NO: 222).

| (SEQ ID NO: 222) | |
|---|---|
| 1 | maeapaspap lspleveldp efepqsrprs ctwplqrpel qaspakpsge taadsmipee |
| 61 | eddeddedgg gragsamaig ggggsgtlgs gllledsarv lapggqdpgs gpataaggls |
| 121 | ggtqallqpq qplpppqpga aggsgqprkc ssrrnawgnl syadlitrai esspdkrltl |
| 181 | sqiyewmvrc vpyfkdkgds nssagwknsi rhnlslhsrf mrvqnegtgk sswwiinpdg |
| 241 | gksgkaprrr aysmdnsnky tksrgraakk kaalqtapes addspsqlsk wpgsptsrss |
| 301 | deldawtdfr srtnsnastv sgrlspimas teldevqddd aplspmlyss saslspsvsk |
| 361 | pctvelprlt dmagtmnlnd gltenlmddl ldnitlppsq psptgglmqr sssfpyttkg |
| 421 | sglgsptssf nstvfgpssl nslrqspmqt iqenkpatfs smshygnqtl qdlltsdsls |
| 481 | hsdvmmtqsd plmsqastav saqnsrrnvm lrndpmmsfa aqpnqgslvn qnllhhqhqt |
| 541 | qgalggsral snsvsnmgls essslgsakh qqqspvsqsm qtlsdslsgs slystsanlp |
| 601 | vmghekfpsd ldldmfngsl ecdmesiirs elmdadgldf nfdslistqn vvglnvgnft |
| 661 | gakqassqsw vpg |

The mRNA sequence encoding human MiR-24 provided by Genbank Accession No. AF480527.1, is incorporated herein by reference, and is shown below (SEQ ID NO: 223).

| (SEQ ID NO: 223) | |
|---|---|
| 1 | tggctcagtt cagcaggaac ag |

The mRNA sequence encoding human MiR-125a-5p (hsa-mir-125a) provided by Genbank Accession No. LM608509.1, is incorporated herein by reference, and is shown below (SEQ ID NO: 224).

| | |
|---|---|
| 1 | tgccagtctc taggtccctg agacccttta acctgtgagg acatccaggg tcacaggtga |
| 61 | ggttcttggg agcctggcgt ctggcc |

The mRNA sequence encoding human MiR-203a (MiR-203), provided by Genbank Accession No. NR_029620.1, is incorporated herein by reference, and is shown below (SEQ ID NO: 225).

| | |
|---|---|
| 1 | gtgttgggga ctcgcgcgct gggtccagtg gttcttaaca gttcaacagt tctgtagcgc |
| 61 | aattgtgaaa tgtttaggac cactagaccc ggcgggcgcg gcgacagcga |

The mRNA sequence encoding human MiR-140, provided by Genbank Accession No. NR_029681.1, is incorporated herein by reference, and is shown below (SEQ ID NO: 226).

| | |
|---|---|
| 1 | tgtgtctctc tctgtgtcct gccagtggtt ttaccctatg gtaggttacg tcatgctgtt |
| 61 | ctaccacagg gtagaaccac ggacaggata ccggggcacc |

The mRNA sequence encoding human MiR-27a, provided by Genbank Accession No. NR_029501.1, is incorporated herein by reference, and is shown below (SEQ ID NO: 227).

| | |
|---|---|
| 1 | ctgaggagca gggcttagct gcttgtgagc agggtccaca ccaagtcgtg ttcacagtgg |
| 61 | ctaagttccg ccccccag |

The mRNA sequence encoding human miR-181a, provided by Genbank Accession No. NR_029611.1 and is shown below (SEQ ID NO: 228).

| | |
|---|---|
| 1 | agaagggcta tcaggccagc cttcagagga ctccaaggaa cattcaacgc |
| 51 | tgtcggtgag tttgggattt gaaaaaacca ctgaccgttg actgtacctt |
| 101 | ggggtcctta |

The mRNA sequence encoding mouse miR-181a, provided by Genbank Accession No. NR_029568.1, is incorporated herein by reference, and is shown below (SEQ ID NO: 231).

| | |
|---|---|
| 1 | ccatggaaca ttcaacgctg tcggtgagtt tgggattcaa aaacaaaaaa |
| 51 | accaccgacc gttgactgta ccttgg |

### Formulation and Dosing

In accordance with certain examples, a pharmaceutical composition of the invention is formulated to be compatible with its intended route of administration. For treatment of autoimmune disease, administration of cargo-loaded nanopieces (complexes) is carried out by systemic administration, e.g., intravenously. In some examples, the compositions may be administered by injection or infusion into a localized tissue site, e.g., into an articulating joint or by inhalation, transdermally, orally, rectally, transmucosally, intestinally, parenterally, intramuscularly, intra-articularly, subcutaneously, intravenously or other suitable methods that will be readily selected by the person of ordinary skill in the art, given the benefit of this disclosure. For example, solutions or suspensions used for parenteral, intradermal, intra-articular, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfate; chelating agents such as ethylenediamine tetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral and/or intra-articular preparation can be enclosed in ampules, disposable syringes or multiple dose vials made of glass or plastic.

A biologically acceptable medium includes, but is not limited to, any and all solvents, dispersion media, and the like which may be appropriate for the desired route of administration of the complexes of the present disclosure. The use of such media for pharmaceutically active substances is known in the art. Except insofar as any conventional media or agent is incompatible with the activity of the small molecule, protein, polypeptide and/or peptide, its use in the pharmaceutical preparation of the invention is contemplated. Suitable vehicles and formulations are described, for example, in the book Remington's Pharmaceutical Sciences (Remington's Pharmaceutical Sciences. Mack Publishing Company, Easton, PA, USA 1985). These vehicles include injectable formulations.

The complexes of the present invention may be administered by any suitable route. For example, a pharmaceutical preparation may be administered in tablets or capsules, by injection, by infusion, by inhalation, topically (e.g., by lotion or ointment), by suppository, by controlled release patch, or the like.

The complexes described herein may be administered to an individual (e.g., a human or animal such as a non-human primate) for therapy by any suitable route of administration, including intravenously, orally, nasally, rectally, intravaginally, parenterally, intra-articularly, intracisternally, topically, buccally, sublingually, epidurally and the like. Intra-articular administration is useful for local treatment of disease and flare-up, e.g. pain in joints, synovitis and the like.

Regardless of the route of administration selected, the pharmaceutical compositions of the present invention are formulated into pharmaceutically acceptable dosage forms such as described below or by other conventional methods known to those of skill in the art. Actual dosage levels of the pharmaceutical compositions described herein may be varied so as to obtain an amount of the compound which is effective to achieve the desired therapeutic response for a particular patient, composition, and mode of administration, without being toxic to the patient.

Joint disease is treated using the complexes or compositions described herein. For example, methods are provided for treating a patient having a joint disease, by administering to the patient a therapeutically effective amount of a complex or composition of the present invention. For in vivo therapies based on local injection (e.g., intratumoral, intraarticularly, intramuscularly, into the peritoneal cavity, and aerosolized treatments) the RNT/small RNA complex is advantageously water soluble and so may be administered as an aqueous injection.

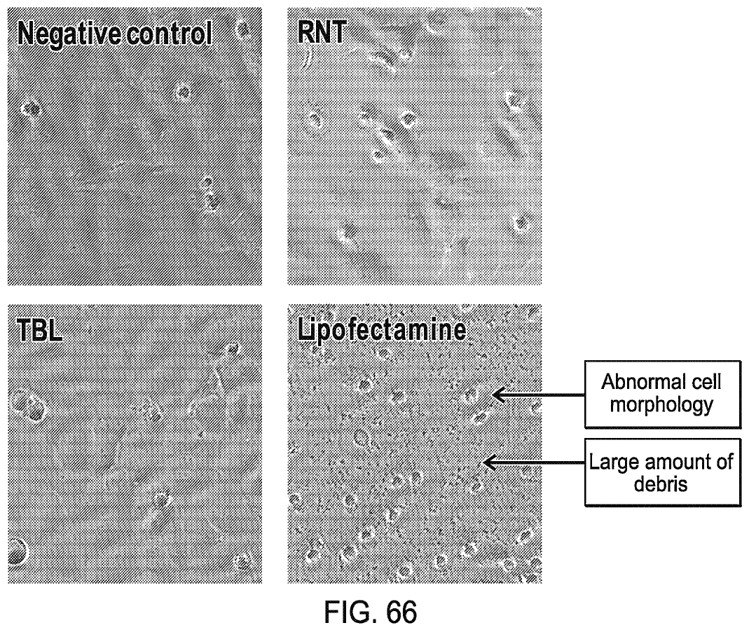

The selected dosage level will depend upon a variety of factors including the activity of a particular compound or ester, salt or amide thereof, the route of administration, the time of administration, the rate of excretion of the particular compound being employed, the duration of the treatment, other drugs, compounds and/or materials used in combination with the particular complex employed, the age, sex, weight, condition, general health and prior medical history of the patient being treated, and like factors well known in the medical arts. A physician, veterinarian or research scientist having ordinary skill in the art can readily determine and prescribe the effective amount of the pharmaceutical composition required. For example, the physician, veterinarian or research scientist could start doses of the compounds of the invention employed in the pharmaceutical composition at levels lower than that required in order to achieve the desired therapeutic effect and gradually increase the dosage until the desired effect is achieved. Furthermore, different delivery materials are used to administer different doses and dose ranges. For example, Nanopieces demonstrate good biocompatibility and low toxicity. Previous studies have demonstrated no significant toxicity with an administration of 25 μg delivery nanotubes (RNTs) in vivo (Journeay W S, et al. Int J Nanomedicine. 2008; 3(3):373-83). Even with a 50 μg dose, inflammation that resulted from RNTs was resolved after 7 days. In comparison, some conventional delivery materials such as carbon nanotubes, can cause inflammation at much lower doses the resulting in inflammation that can last for two months. In the current system, a 5 μg dose of RNT in Nanopiece was effective in the delivery of cargo. Therefore, the effective doses of RNT Nanopieces are significantly lower than their toxic doses, providing a good therapeutic index. Moreover, RNTs or TBLs showed a lower toxicity than lipid-based delivery vehicles. In FIG. 66, ATDC5 cells were cultured with no additives (negative control), Nanopieces of 0.1 nmol non-targeting siRNA with 10 μg of RNT, Nanopieces of 0.1 nmol non-targeting siRNA with 2.5 μg TBL, or 0.1 nmol non-targeting siRNA with 6 μg Lipofectamine 2000. After 24 hours, ATDC5 cells cultured with Lipofectamine 2000 showed abnormal cell morphology and large amount of cell debris, however, cells cultured with either RNT nanopiece or TBL nanopiece presented normal morphology as the negative control.

In general, a suitable daily dose of a compound of the invention will be that amount of the compound which is the lowest dose effective to produce a therapeutic effect. Such an effective dose will generally depend upon the factors described above. Generally, doses of the compounds of this invention for a patient will range from about 0.0001 to about 100 mg per kilogram of body weight per day, or from about 0.001 to 30 mg/kg body weight, from about 0.01 to 25 mg/kg body weight, from about 0.1 to 20 mg/kg body weight, from about 1 to 10 mg/kg, from about 2 to 9 mg/kg, from about 3 to 8 mg/kg, from about 4 to 7 mg/kg, or from about 5 to 6 mg/kg body weight.

The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of biologically active agent can include a single treatment or, preferably, can include a series of treatments. It will also be appreciated that the effective dosage for treatment may increase or decrease over the course of a particular treatment. Changes in dosage may result from the results of diagnostic assays.

If desired, the effective daily dose of the active compound may be administered as one, two, three, four, five, six or more sub-doses administered separately at appropriate intervals throughout the day, optionally, in unit dosage forms. In certain embodiments, an effective dose is given every other day, twice a week, once a week or once a month.

A complex of the invention can be administered as such or in admixtures with pharmaceutically acceptable carriers and can also be administered in conjunction with other antimicrobial agents such as penicillin, cephalosporin, aminoglycosides, glycopeptides and the like. Conjunctive therapy includes sequential, simultaneous and separate administration of an active compound in such a way that the therapeutic effects of the first administered compound are still present when a subsequent administration is performed.

Another aspect of the present disclosure provides pharmaceutically acceptable compositions comprising a therapeutically effective amount of one or more of the complexes described herein, formulated together with one or more pharmaceutically acceptable carriers (additives) and/or diluents. As described in detail below, the pharmaceutical compositions of the present disclosure may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection or intraarticularly as, for example, a sterile solution or suspension; (3) topical application, for example, as a cream, ointment or spray applied to the skin; or (4) intravaginally or intrarectally, for example, as a pessary, cream or foam. However, in certain embodiments the subject complexes may be simply dissolved or suspended in sterile water.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in compositions of the present invention.

Examples of pharmaceutically acceptable antioxidants include but are not limited to: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention may conveniently be presented in unit dosage form and may be prepared by any methods well known in the pharmaceutical art. The amount of active ingredient which can be combined with a carrier material to produce a single dosage form will vary depending upon the individual being treated and the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, from about 5 percent to about 70 percent, from about 10 percent to about 30 percent, from about 15 percent to about 25 percent, or from about 18 percent to about 22 percent. In an alternative embodiment, compounds of the present invention can be administered per se, e.g., in the absence of carrier material.

Methods of preparing the formulations or compositions of the present invention include the step of associating a complex described herein with a carrier and, optionally, one or more accessory ingredients. In general, the formulations are prepared by uniformly associating a complex of the present invention with liquid carriers, finely divided solid carriers, or both, and, optionally, shaping the product.

Formulations suitable for oral administration may be in the form of capsules, cachets, pills, tablets, lozenges (using a flavored basis, such as sucrose and acacia or tragacanth), powders, granules, as a solution or a suspension in an aqueous or non-aqueous liquid, as an oil-in-water or water-in-oil liquid emulsion, as an elixir or syrup, as pastilles (using an inert base, such as gelatin and glycerin, or sucrose and acacia) and/or as mouth washes and the like, each containing a predetermined amount of a complex of the present invention as an active ingredient. A complex of the present invention may also be administered as a bolus, electuary or paste.

Ointments, pastes, creams and gels may contain, in addition to a complex of the present disclosure, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a complex of the present disclosure, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a complex of the present invention to the body. Such dosage forms can be made by dissolving or dispersing the complex in the proper medium. Absorption enhancers can also be used to increase the flux of the complex across the skin. The rate of such flux can be controlled by either providing a rate controlling membrane or dispersing the complex in a polymer matrix or gel.

Pharmaceutical compositions of this invention suitable for parenteral administration comprise one or more complexes of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol asorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Injectable depot forms are made by forming microencapsule matrices of the complexes in biodegradable polymers such as polylactide-polyglycolide. Depending on the ratio of drug to polymer, and the nature of the particular polymer employed, the rate of drug release can be controlled. Examples of other biodegradable polymers include poly (orthoesters) and poly (anhydrides). Depot injectable formulations are also prepared by entrapping the drug in liposomes or microemulsions which are compatible with body tissue.

In accordance with certain examples, complexes of the present invention can be incorporated into pharmaceutical compositions suitable for administration. Such compositions typically comprise the complexes disclosed here and a pharmaceutically acceptable carrier. As used herein the term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

In accordance with certain examples, a pharmaceutical composition of the invention is formulated to be compatible with its intended route of administration. Such pharmaceutical compositions may be administered by inhalation, intraarticularly, transdermally, orally, rectally, transmucosally, intestinally, parenterally, intramuscularly, subcutaneously, intravenously or other suitable methods that will be readily selected by the person of ordinary skill in the art, given the benefit of this disclosure. For example, solutions or suspensions used for parenteral, intraarticularly, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

The present disclosure is directed to methods of forming a delivery complex, for example, by mixing one or more agents with fully formed rosette nanotubes or modules that self-assemble into rosette nanotubes, such as the compounds of Formula I or Formula II. According to one aspect, fully formed rosette nanotubes in the form of a powder is dissolved in water and heated to boiling. The solution is then cooled to room temperature. One or more agents is then added to the solution of nanotubes at a suitable temperature and for a suitable period of time until a complex of the nanotube and one or more agents forms. Suitable ratios of the nucleic acid to nanotube include about 0.01:1 (wt/wt) to about 1:0.1 (wt/wt).

### Definitions

“Alkyl”, as used herein, refers to the radical of saturated or unsaturated aliphatic groups, including straight-chain alkyl, alkenyl, or alkynyl groups, branched-chain alkyl, alkenyl, or alkynyl groups, cycloalkyl, cycloalkenyl, or cycloalkynyl (alicyclicf) groups, alkyl substituted cycloalkyl, cycloalkenyl, or cycloalkynyl groups, and cycloalkyl substituted alkyl, alkenyl, or alkynyl groups. Unless otherwise indicated, a straight chain or branched chain alkyl has 30 or fewer carbon atoms in its backbone (e.g., C₁-C₃₀ for straight chain, C₃-C₃₀ for branched chain), more preferably 20 or fewer carbon atoms, more preferably 12 or fewer carbon atoms, and most preferably 8 or fewer carbon atoms. Likewise, preferred cycloalkyls have from 3-10 carbon atoms in their ring structure, and more preferably have 5, 6 or 7 carbons in the ring structure. The ranges provided above are inclusive of all values between the minimum value and the maximum value.

The term “alkyl” includes both “unsubstituted alkyls” and “substituted alkyls”, the latter of which refers to alkyl moieties having one or more substituents replacing hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, a phosphinate, amino, amino, amidine, imine, cyano, nitro, azido, sulfhydryl, alkylthio, sulfate, sulfonate, sulfamoyl, sulfonamido, sulfonyl, heterocyclyl, aralkyl, or an aromatic or heteroaromatic moiety.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Preferred alkyl groups are lower alkyls.

The alkyl groups may also contain one or more heteroatoms within the carbon backbone. Preferably the heteroatoms incorporated into the carbon backbone are oxygen, nitrogen, sulfur, and combinations thereof. In certain embodiments, the alkyl group contains between one and four heteroatoms.

“Alkenyl” and “Alkynyl”, as used herein, refer to unsaturated aliphatic groups containing one or more double or triple bonds analogous in length (e.g., C₂-C₃₀) and possible substitution to the alkyl groups described above.

“Halogen”, as used herein, refers to fluorine, chlorine, bromine, or iodine.

The term “optionally substituted” as used herein, refers to all permissible substituents of the compounds described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aryloxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, and polypeptide groups.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc.

The term “amino acid” is inclusive of the 20 common amino acids, as well as “nonstandard amino acids,” for example, D-amino acids and chemically (or biologically) produced derivatives of “common” amino acids, including for example, β-amino acids. Accordingly, amino acids according to the present disclosure include the commonly known amino acids such as glycine (Gly, G), alanine (Ala, A), valine (Val, V), leucine (Leu, L), isoleucine (Ile, I), proline (Pro, P), hydroxyproline, phenylalanine (Phe, F), tyrosine (Tyr, Y), tryptophan (Trp, W) cysteine (Cys, C), methionine (Met, M) serine (Ser, S), o-phosphoserine, threonine (Thr, T), lysine (Lys, K), arginine (Arg, R), histidine (His, H), aspartate (Asp, D), glutamate (Glu, E), γ-carboxyglutamate, asparagine (Asn, N), glutamine (Gln, Q) and the like. Amino acids also include stereoisomers thereof and compounds structurally similar to the amino acids or modifications or derivatives thereof. Exemplary amino acids within the scope of the present disclosure include lysine, arginine, serine, glycine, aspartate and the like. The amino acids of the present disclosure are modified only at their terminal amine group.

Aminoe acids are composed of amine (—NH₂) and carboxylic acid (—COOH) functional groups, along with a side-chain specific to each amino acid. The key elements of an amino acid are carbon, hydrogen, oxygen, and nitrogen, though other elements are found in the side-chains of certain amino acids.

In the structure shown below, Z represents a side-chain specific to each amino acid. The carbon atom next to the carboxyl group (which is therefore numbered 2 in the carbon chain starting from that functional group) is called the a—carbon. Amino acids containing an amino group bonded directly to the alpha carbon are referred to as alpha amino acids.

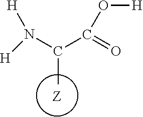

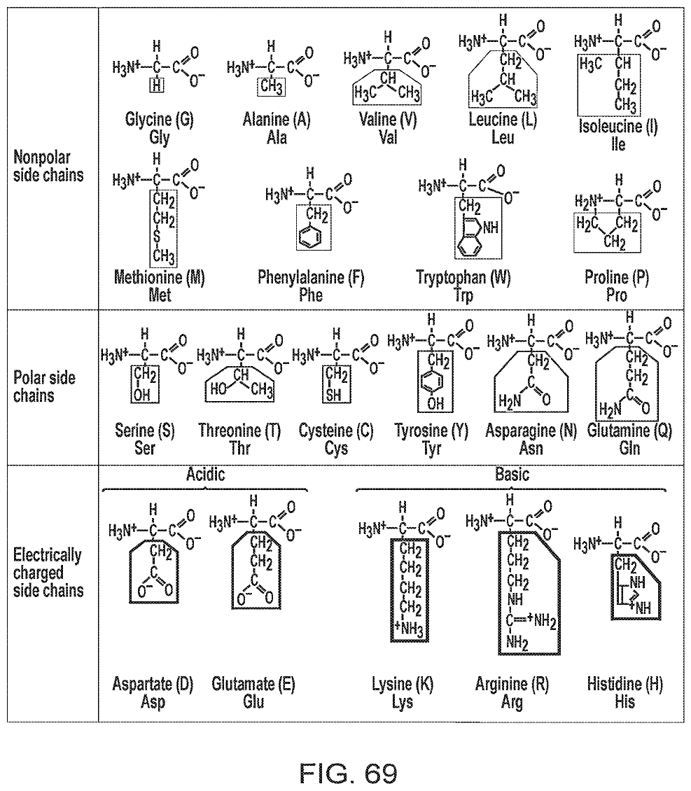

Amino acids can be divided into amino acid containing hydrophilic side chains, hydrophobic side chains, and electrically charged side chains. See FIG. 69, wherein the side chains are shaded.

The term “peptide” is inclusive of both straight and branched amino acid chains, as well as cyclic amino acid chains, which comprise at least 2 amino acid residues. The terms “peptide” and “polypeptide” are used interchangeably herein. Accordingly, polypeptides according to the present disclosure include two or more amino acids covalently linked together. According to one aspect, the two or more amino acids are covalently linked together at least in part by one or more peptide bonds. The polypeptides of the present disclosure are modified only at their terminal amine group. For example, the peptide or fragment of a full-length protein comprises 2, 5, 10, 50, 100, 200, 500 600, 700, 750, 800, 900, 1000 or more amino acids in length or up to the full length of a reference protein.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the invention may comprise natural nucleic acid sequences and variants thereof, artificial nucleic acid sequences, or a combination of such sequences. As used herein, one of skill in the art will understand that the term “nucleic acid probe” includes probes known as molecular beacons which include synthetic oligonucleotide hybridization probes that can report the presence of specific nucleic acids in homogenous solutions or in cells. Species of molecular beacons include hairpin shaped molecules with a detectable marker such as an internally quenched fluorophore whose fluorescence is restored when they bind to a target nucleic acid sequence. Technically, molecular beacons can be designed to target any gene and can be linked with fluorescent molecules of different fluorescence wavelengths.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

Examples of modified nucleotides include, but are not limited to 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine and the like. Nucleic acid molecules may also be modified at the base moiety, sugar moiety or phosphate backbone.

The term “small RNA” is used as it is in the art, and includes a duplex of RNA (30 bases or less in each strand) that targets mRNA. Small RNA may be chemically or enzymatically synthesized. Small RNA in accordance with the present invention may be incorporated and then activated in RISC (RNA-induced silencing complex).

A “therapeutically effective amount” is an amount necessary to prevent, delay or reduce the severity of the onset of disease, or an amount necessary to arrest or reduce the severity of an ongoing disease, and also includes an amount necessary to enhance normal physiological functioning.

The word “transfect” is broadly used herein to refer to introduction of an exogenous compound, such as a polynucleotide sequence, into a prokaryotic or eukaryotic cell; the term includes, without limitation, introduction of an exogenous nucleic acid into a cell, which may result in a permanent or temporary alteration of genotype in an immortal or non-immortal cell line. Accordingly, embodiments of the present disclosure include the introduction of a polynucleotide sequence to either be expressed or to inhibit expression of a target gene.

As may be used herein, the terms “drug,” biologically active agent,” and “therapeutic agent” are used interchangeably and are intended to include, but are not limited to, those compounds recognized by persons of skill in the art as being biologically active agents, or drugs or therapeutic agents and include any synthetic or natural element or compound which when introduced into the body causes a desired biological response, such as altering body function.

As used herein, the terms “parenteral administration” and “administered parenterally” are intended to include, but are not limited to, modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal injection, intrasternal injection, infusion and the like.

As used herein, the terms “systemic administration,” “administered systemically,” “peripheral administration” and “administered peripherally” are intended to include, but are not limited to, the administration of a compound, drug or other material other than directly into the central nervous system, such that it enters an individual's system and, thus, is subject to metabolism and other like processes, such as, for example, subcutaneous administration.

The term “treatment,” as used herein, is intended to include, but is not limited to, prophylaxis, therapy and cure. A patient or individual receiving treatment is any animal in need, such as humans, non-human primates, and other mammals such as horses, camels, cattle, swine, sheep, poultry, goats, rabbits, mice, guinea pigs, dogs, cats and the like.

As used herein, the term “therapeutically effective amount” is intended to include, but is not limited to, an amount of a compound, material, or composition comprising a complex of the present invention which is effective for producing a desired therapeutic effect in at least a subpopulation of cells in an animal and thereby altering (e.g., reducing or increasing) the biological consequences of one or more pathways in the treated cells, at a reasonable benefit/risk ratio.

As used herein, the term “pharmaceutically acceptable” is intended to include, but is not limited to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, a “pharmaceutically acceptable agent” (such as a salt, carrier, excipient or diluent) is a component which (1) is compatible with the RNT/small RNA composites in that it can be included in the delivery composition without eliminating the capacity of the RNT/small RNA composites to transfect cells and deliver small RNA; and (2) where the delivery composition is intended for therapeutic uses, is suitable for use with an animal (e.g., a human) without undue adverse side effects, such as toxicity, irritation, and allergic response. Side effects are “undue” when their risk outweighs the benefit provided by the pharmaceutical agent.

As used herein, the term “pharmaceutically acceptable carrier” is intended to include, but is not limited to, a pharmaceutically acceptable material, composition or vehicle, such as a liquid or solid filler, diluent, excipient, solvent or encapsulating material, involved in carrying or transporting the complexes of the present disclosure from one organ, or portion of the body, to another organ, or portion of the body. Each carrier must be “acceptable” in the sense of being compatible with the other ingredients of the formulation and not unduly dangerous to the patient. Examples of materials which can serve as pharmaceutically acceptable carriers include but are not limited to: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations, which could easily be determined by one of skill in the art.

Chemical compounds, polynucleotides, polypeptides, and oligosaccharides of the invention are purified and/or isolated. Purified defines a degree of sterility that is safe for administration to a human subject, e.g., lacking infectious or toxic agents. Specifically, as used herein, an “isolated” or “purified” compound, nucleic acid molecule, polynucleotide, polypeptide, protein or oligosaccharide, is substantially free of other cellular material, or culture medium when produced by recombinant techniques, or chemical precursors or other chemicals when chemically synthesized. For example, purified compositions are at least 60% by weight (dry weight) the compound of interest. Preferably, the preparation is at least 75%, more preferably at least 90%, and most preferably at least 99%, by weight the compound of interest. Purity is measured by any appropriate standard method, for example, by column chromatography, thin layer chromatography, or high-performance liquid chromatography (HPLC) analysis. For example, a purified compound refers to a one that has been separated from other proteins, lipids, and nucleic acids with which it is naturally associated. Preferably, the compound constitutes at least 10, 20, 50, 70, 80, 90, 95, 99-100% by dry weight of the purified preparation.

As used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) or polypeptide is free of the amino acid sequences or nucleic acid sequences that flank it in its naturally-occurring state. Purified also defines a degree of sterility that is safe for administration to a human subject, e.g., lacking infectious or toxic agents.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

By “isolated nucleic acid” is meant a nucleic acid that is free of the genes which, in the naturally-occurring genome of the organism from which the DNA of the invention is derived, flank the gene. For example, the purified or isolated nucleic acid is a siRNA. The term covers, e.g., (a) a DNA which is part of a naturally occurring genomic DNA molecule, but is not flanked by both of the nucleic acid sequences that flank that part of the molecule in the genome of the organism in which it naturally occurs; (b) a nucleic acid incorporated into a vector or into the genomic DNA of a prokaryote or eukaryote in a manner, such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Isolated nucleic acid molecules according to the present invention further include molecules produced synthetically as well as any nucleic acids that have been altered chemically and/or that have modified backbones.

A small interfering RNA (siRNA), also known as short interfering RNA or silencing RNA, is a class of double-stranded RNA molecules, 20-25 base pairs in length, similar to miRNA, and operating within the RNA interference (RNAi) pathway. It interferes with the expression of specific genes with complementary nucleotide sequences by degrading mRNA after transcription, preventing translation. They are produced from dsRNA or hairpin looped RNA which, after entering a cell is split by an RNase III-like enzyme, called Dicer, using RNase or restriction enzymes. The siRNA may then incorporated into a multi-subunit protein complex called RNAi-induced silencing complex (RISC).

As used therein, the term “patient” is intended to include a mammal suffering from a disease. Such a mammal can be a human or another animal such as a companion animal (e.g., dog or cat) or a performance animal or livestock animal (e.g., an equine, bovine, porcine animal).

### EXAMPLES

The following examples are specific embodiments of the present invention but are not intended to limit it.

### Example 1

Nanopieces that include RNTs and exemplary cargo or payload compounds were manufactured. Cargo agents assemble with RNTs into Nanopieces. Then, taking siRNA Nanopiece as an example, it was demonstrated that Nanopieces can be intentionally processed into different sizes and charge for matrix penetration, e.g. preferential delivery of the cargo to specific tissue types. For example, Nanopieces with a net positive charge were made to deliver payload compounds to negatively charged tissue such as cartilage.

The relation between RNT/siRNA ratio and surface charge was evaluated. Selecting the ratio to result in a net positive charge on Nanopieces, Nanopieces have better binding and longer retention time on negatively charged tissue matrix (e.g., human articular cartilage).

For in vitro and in vivo delivery studies, cartilage was used as an example, because cartilage is an avascular tissue with high matrix component, which is a challenging tissue for drug delivery. Other target matrix and/or tissue can be used and the net charge of the Nanopiece tuned for preferential targeting to a selected tissue. It was shown that the processed Nanopieces were efficiently delivered into cartilage matrix from various species, as well as inside chondrocytes. The delivered Nanopieces were fully functional. A composite of polyethylene glycol (PEG) was used to increase Nanopiece delivery efficiency in a protein-rich environment (such as serum). Rat and mouse models showed that the processed Nanopieces successfully achieved trans-matrix and/or tissue delivery in vivo.

For diagnostics, MMP-13 molecular beacons for disease gene detection were co-delivered with non-targeting scrambled molecular beacons as a non-specific signal negative control and GAPDH molecular beacons as an internal house-keeping gene control. Fluorescence signal was accurately translated into gene expression level exemplary of a non-invasive approach to detect real-time, in-situ gene expression in living animals.

For therapeutics, cytokine (IL-1β) was used to stimulate cartilage degeneration mimicking arthritis, especially rheumatoid arthritis. With Nanopiece delivery of IL-1 receptor siRNA, IL-1 receptor expression was knocked down in chondrocytes in mouse cartilage in vivo, so that cartilage degeneration genes (such as MMP-13, MMP-9) were down-regulated and cartilage anabolic genes (such as Col II) were up-regulated.

Nanopieces were used to deliver ADAMTS-5 siRNA into knee joints of mice with cytokine (IL-1α and retinoic acid) stimulation. Cartilage degeneration was significantly inhibited. To mimic osteoarthritis progression, destabilization of medial meniscus (DMM) was conducted on knee joints of mice. With Nanopiece delivery of ADAMTS-5 siRNA, osteoarthritis progression was prevented. These data indicate the Nanopieces are useful to prevent and/or inhibit cartilage degeneration and arthritis progression.

### Example 2

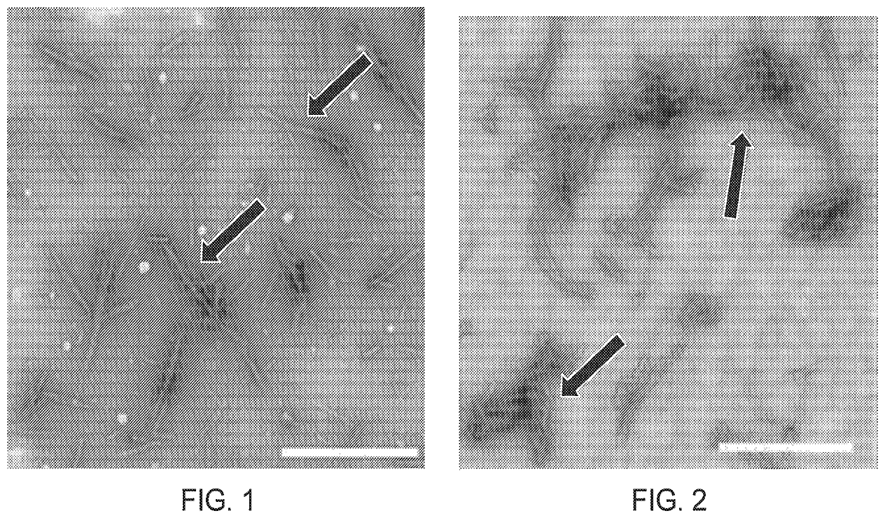

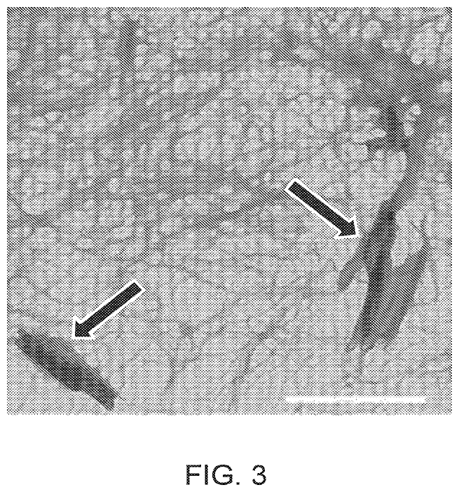

Successful assembly of RNTs into Nanopieces was shown, (see ARROWS) and they were used to deliver various types of cargo reagents including small nucleic acids (siRNA, FIG. 1), long nucleic acids (plasmid DNA, FIG. 2), peptide or protein (Matrilin-3, FIG. 3) as well as small molecules.

### Example 2.1

Nanopieces containing SiRNA as cargo were manufactured as follows. 2 μL of a 50 μM siRNA solution was mixed with 10 μL of a 1 mg/mL RNTs mixture. The resulting mixture was sonicated for 60 s. Dilution factors can range from 1 to 50 μL for preparing the siRNA-RNTs complex mixture and sonication times of the resulting mixture can vary from 1 to 600 s. Results are shown in FIG. 1.

### Example 2.2

Nanopieces containing DNA were manufactured as follows. 0.5 μg DNA was mixed with 10 μL of a 1 mg/mL RNTs solution. The resulting mixture was sonicated for 60 s. Dilution factors can range from 1 to 50 μL for preparing the DNA-RNTs complex mixture and sonication times of the resulting mixture can vary from 1 to 600 s. Results are shown in FIG. 2.

### Example 2.3

Nanopieces containing Matrilin as cargo were manufactured as follows. 10 μL of a 100 μg/mL Matrilin (MATN) protein solution was mixed with 10 μL of a 1 mg/mL RNTs. The resulting mixture was then sonicated for 60 s. Dilution factors can range from 1 to 50 μL for preparing the MATN-RNTs complex mixture and sonication times of the resulting mixture can vary from 1 to 600 s. Results are shown in FIG. 3.

### Example 3

### Design and Processing of Nanopieces

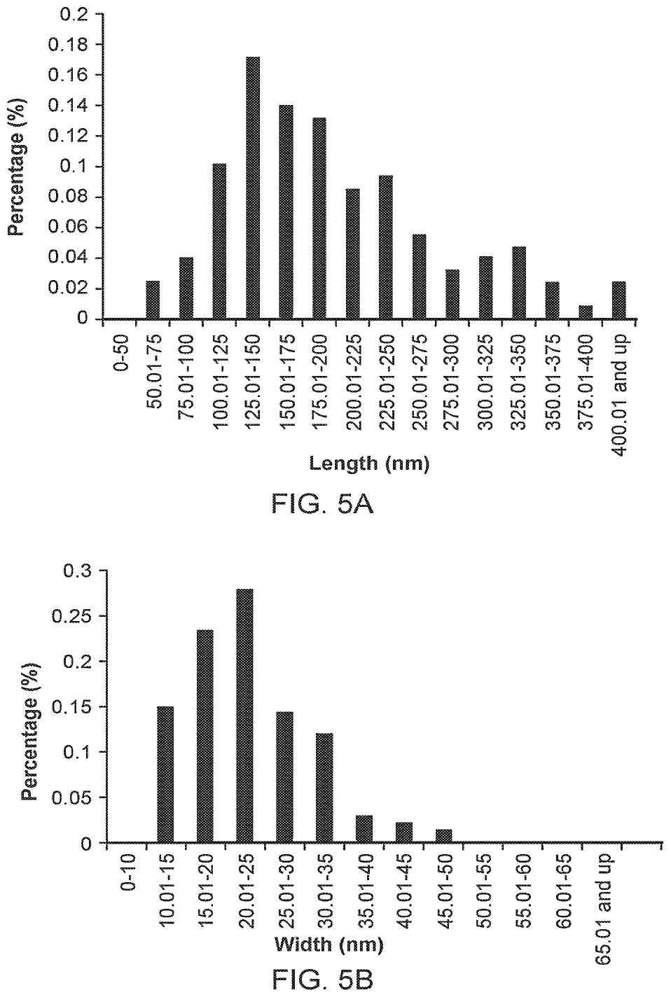

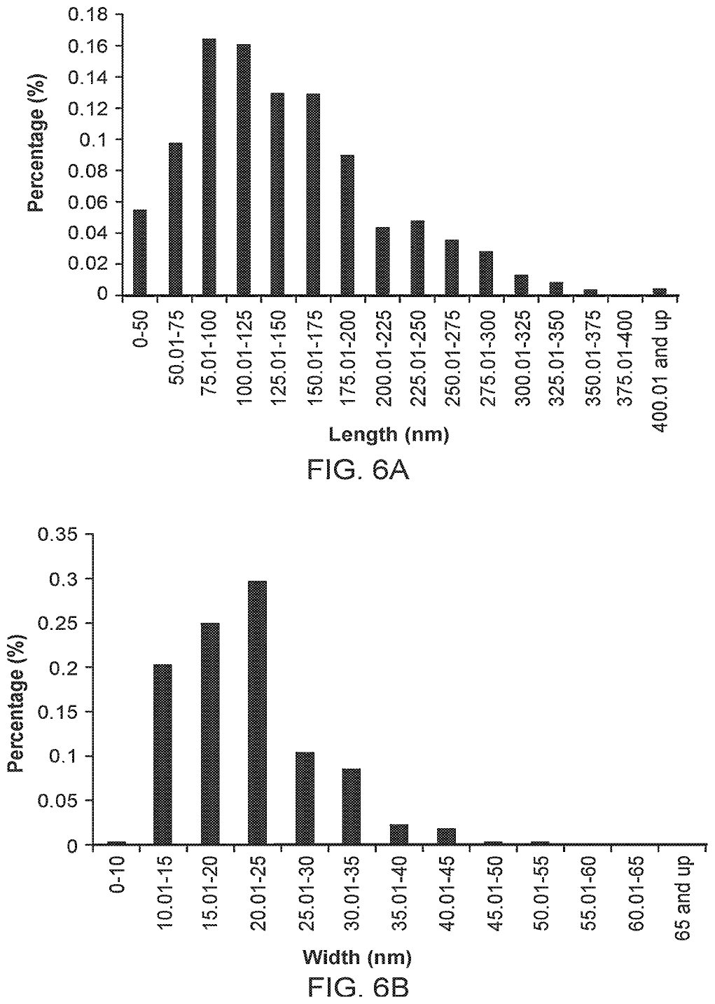

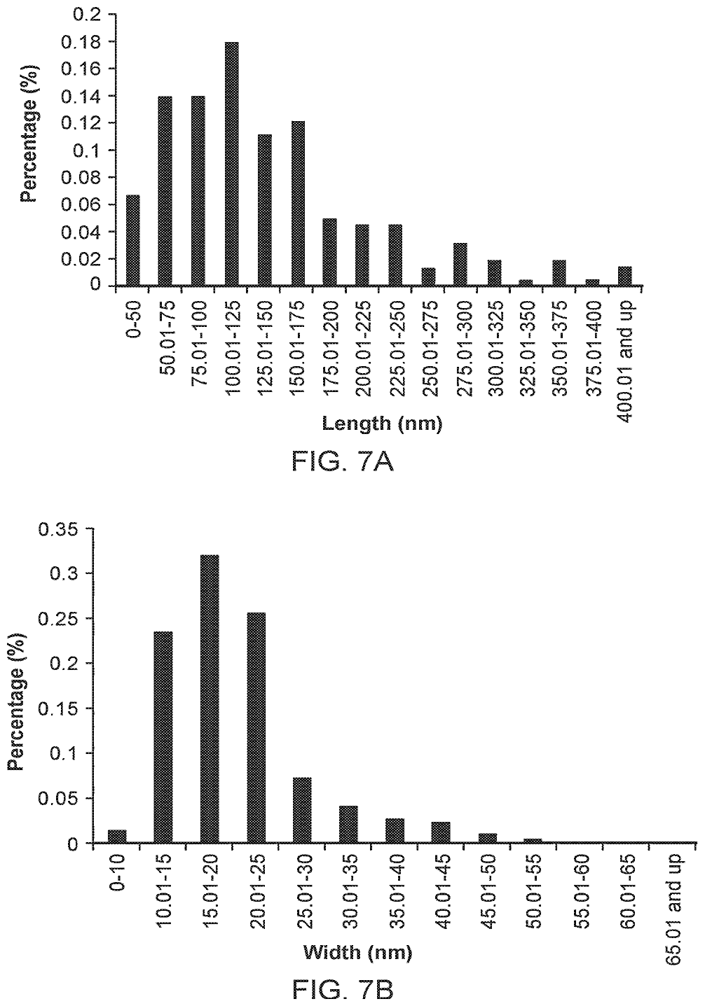

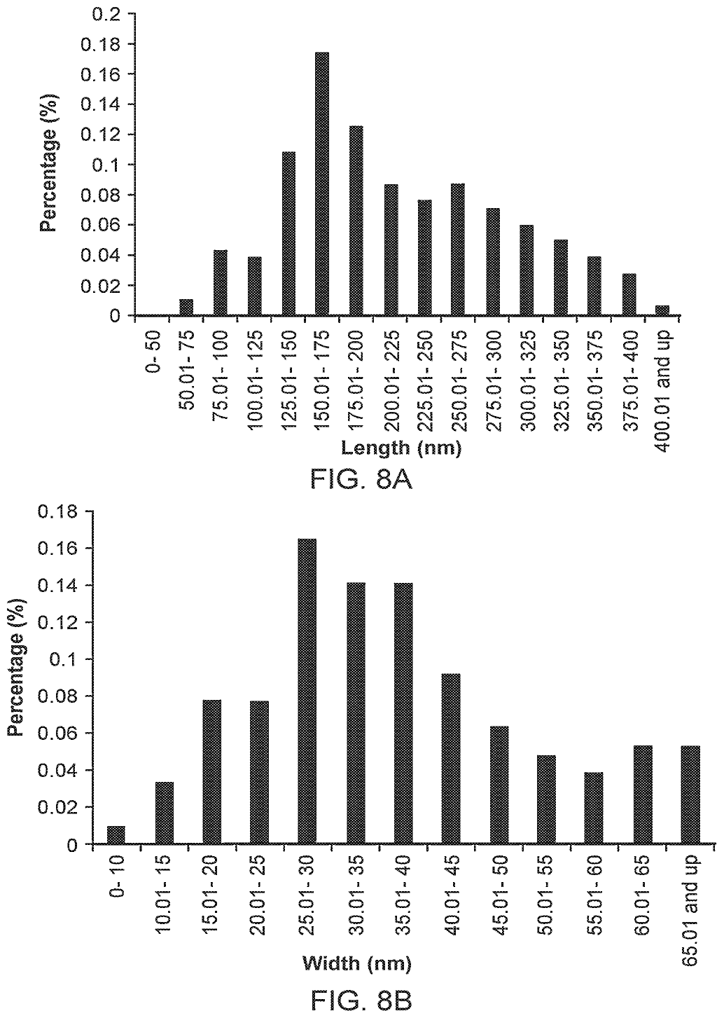

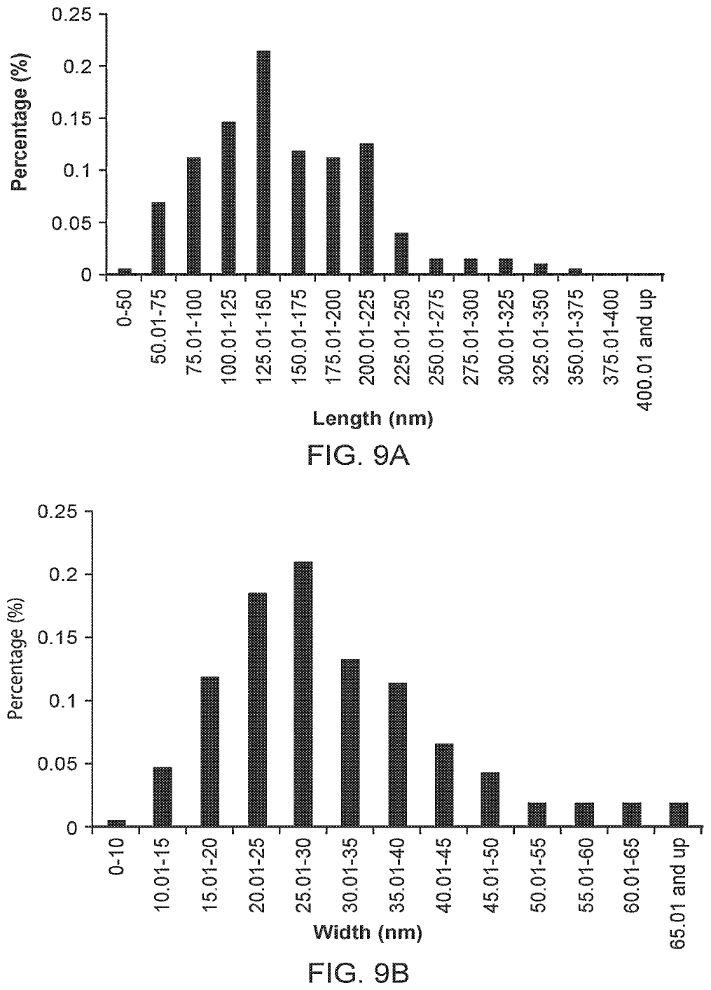

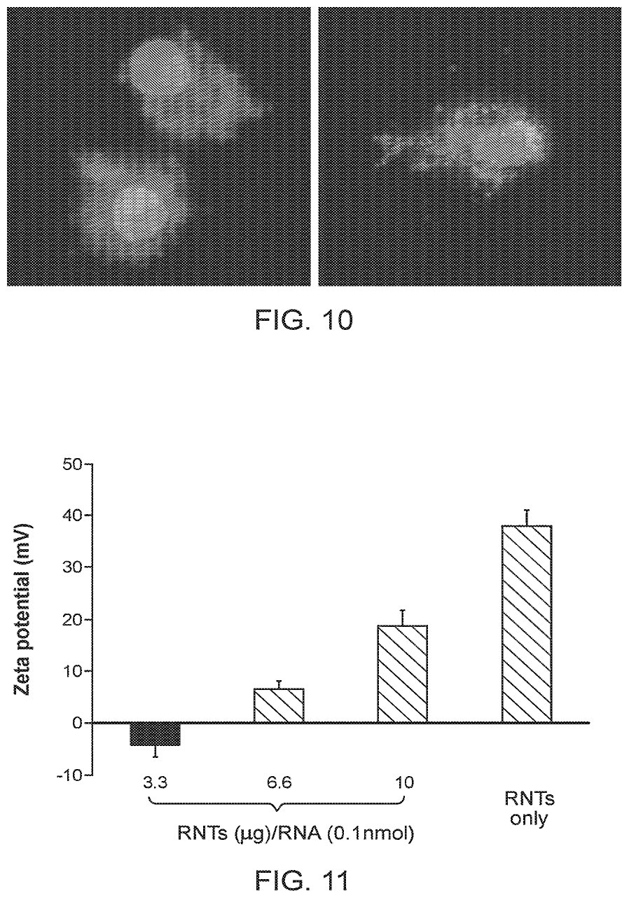

FIG. 4 shows an exemplary assembly mechanism. Processing methods were designed before, during and after assembly to manipulate the sizes of Nanopieces. Taking quench and sonication as examples of processing methods before assembly, FIGS. 6 and 7 demonstrate the formation of smaller Nanopieces compared with those generated under standard conditions (FIG. 5). FIGS. 8 and 9 represent size distributions of examples of processing methods during and after assembly. Small Nanopieces were delivered into cells as shown in FIG. 10.

### Example 3.1

FIGS. 5A-9B demonstrate Nanopieces of different sizes and width that were imaged under a transmission electron microscope, and their length and width were analyzed with Image J software.

Nanopieces of different lengths and widths were prepared using the following exemplary procedures.

### Example 3.1A

5 ug of RNT in 5 uL water was mixed with 50 pmol siRNA in 10 uL water, and then the mixture was sonicated for 2 min to produce Nanopieces (FIGS. 5A and 5B)

### Example 3.1B

5 ug of RNT in 5 uL water is heated to 95° C. for 10 min, and then the solution is immediately putted on ice. After totally cooling down to 0° C., RNT solution is mixed with 50 pmol siRNA in 10 uL water, and then the mixture is sonicated for 2 min to produce Nanopieces (FIGS. 6A and 6B).

### Example 3.1C

5 ug of RNT in 5 uL water is heated to 95° C. for 10 min, and then the solution is immediately subjected to sonication for 5 min. The resulting RNT solution is mixed with 50 pmol siRNA in 10 uL water, and then the mixture is sonicated for 2 min to produce Nanopieces (FIGS. 7A and 7B).

### Example 3.1D

5 ug of RNT in 5 uL in water is mixed with 50 pmol siRNA in 10 uL 0.9% saline, and then the mixture is sonicated for 2 min to produce Nanopieces (FIGS. 8A and 8B).

### Example 3.1E

5 ug of RNT in 5 uL in water is mixed with 50 pmol siRNA in 10 uL water, and then the mixture is sonicated for 4 min to produce Nanopieces (FIGS. 9A and 9B).

### Example 3.2

FIG. 10 shows that fluorescence labeled RNA was delivered into cells using unprocessed and processed Nanopieces. The Nanopieces were added to chondrocytes and the cells were maintained under standard cell culture conditions for 24h. Left Panel of FIG. 10 shows unprocessed nanopieces, while the right panel of FIG. 10 shows processed Nanopieces being delivered into cells.

### Example 3.3

Various types of Nanopieces and their processing methods are described. Nanotubes are converted into nanorods. As shown in FIG. 4, the use of physical methods (sonication, blending, microwave and/or quenching) or chemical methods (altering pH, adding organic solvents, and/or adding of aromatic chemicals) convert nanotubes into homogenous shorter/longer nanorods to result in shorter/longer Nanopieces compared to standard conditions. (FIGS. 5-7). Nanorods were produced via either sonicating RNTs, or heating RNTs to 90° C., and then quenching them on ice. RNTs or Nanorods were used to form Nanopieces. Nanopieces were characterized using transmission electron microscope and their length and width were analyzed with Image J software.

### Example 3.4

Various types of Nanopieces and their processing methods are used to customize the physical characteristics, e.g., length and width, and/or chemical characteristics e.g., surface charge of the delivery vehicle. Two major conditions can be altered: i) assembly conditions (ionic strength, pH and concentration) to achieve Nanopieces with various sizes; and ii) the ratio between nanotubes/nanorods and delivery cargos to achieve different surface charge for the delivery of cargo into different tissues. For example, an increase in ionic strength can be used in the assembly solution to generate longer and wider Nanopieces compared to when using standard conditions (FIG. 4 and FIG. 7). An increase in the ratio of RNTs over siRNA resulted in an increase of the surface positive charge of Nanopieces (FIG. 11). FIG. 8 shows that RNTs and siRNA were dissolved in saline to form Nanopieces as described in the previous sections. Nanopieces were imaged under a transmission electron microscope, and their length and width were analyzed with Image J software. FIG. 11 shows the different ratios of RNTs and siRNA that were used to form Nanopieces. The surface charge (as measured by Zeta potential; mV) of Nanopieces was determined via Nanosizer.

### Example 3.5

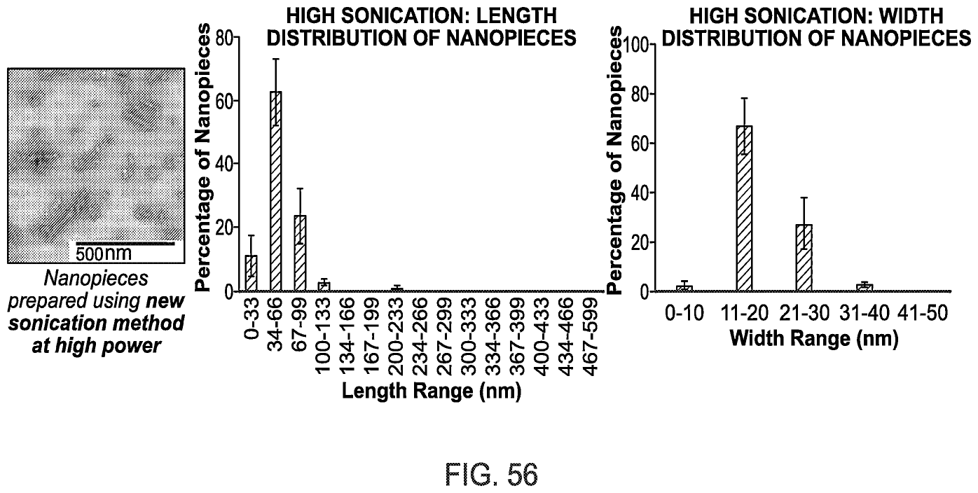

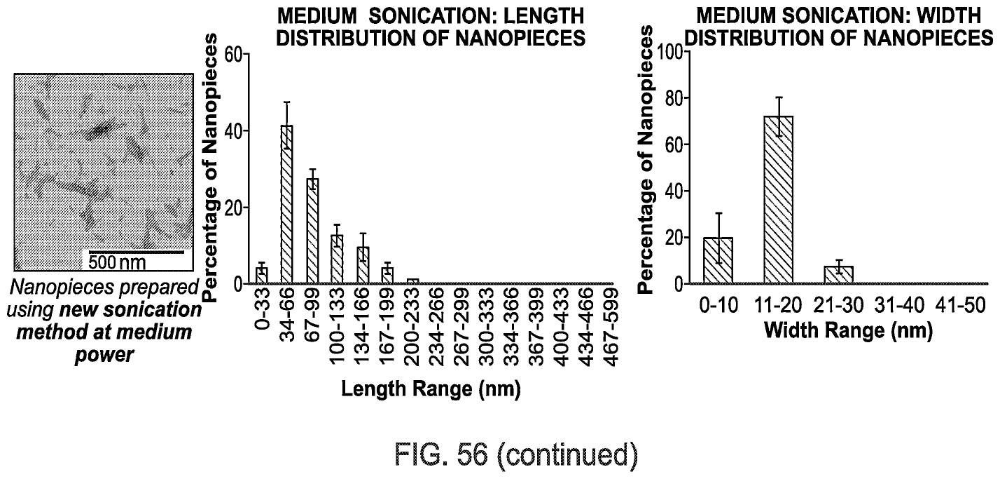

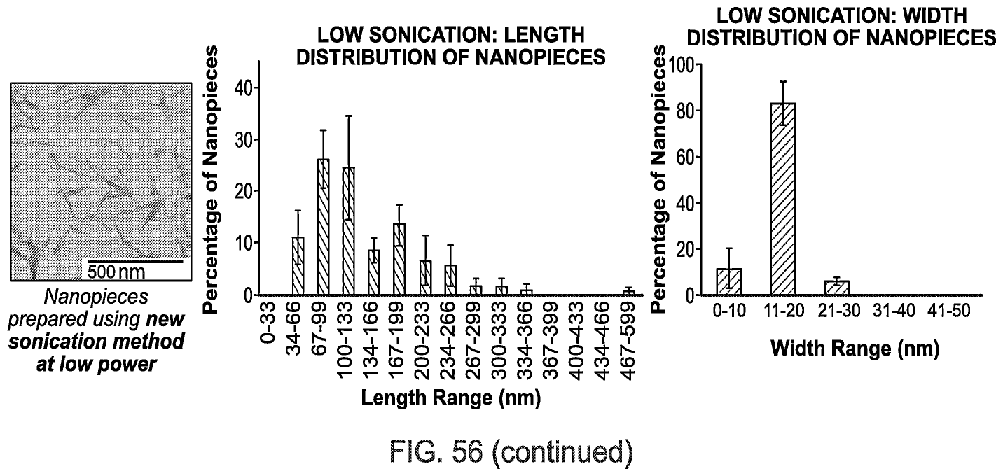

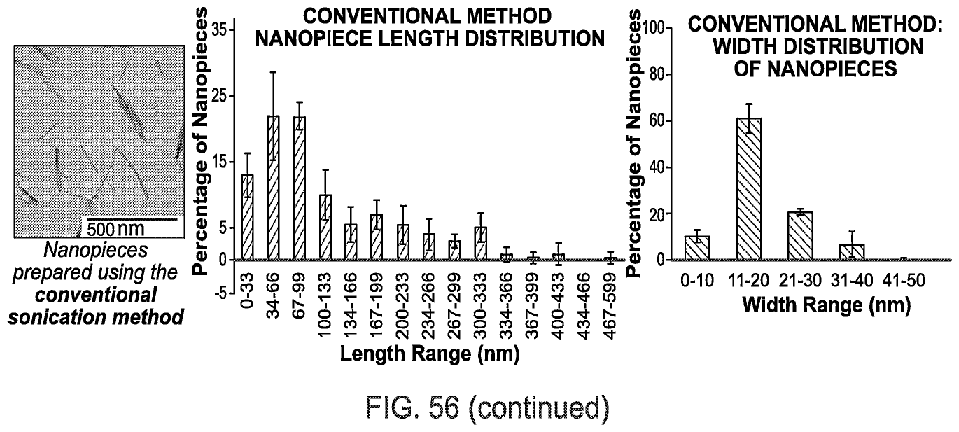

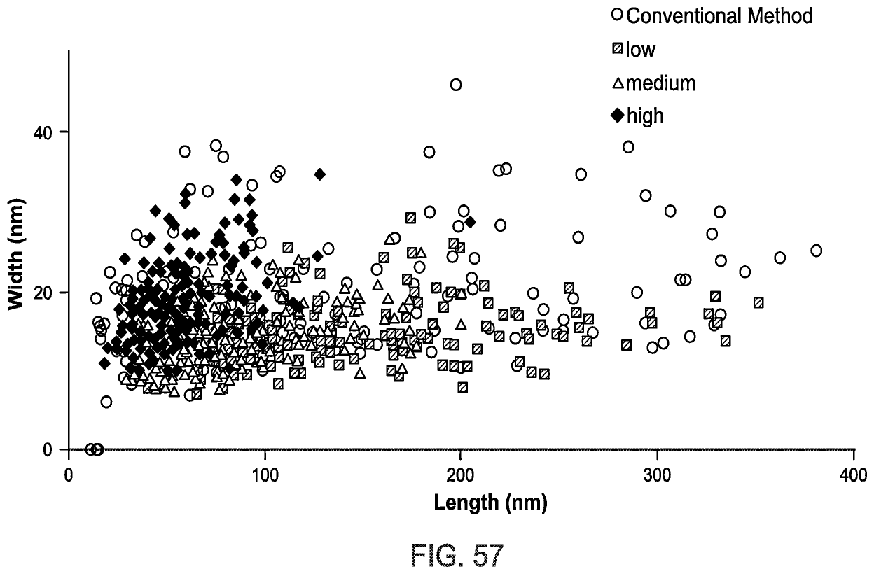

Processing after assembly included physical methods, e.g., using different power of soinication, heating, blending and/or microwave; or chemical methods, like altering of pH and adding of aromatic chemicals. For example, the use of low, medium and high power of sonication resulted in Nanopieces with different size (length) and morphology (aspect ratio, which is equal to length/width) (FIGS. 4, 56, and 57). FIGS. 56-57 shows that Nanopieces were formed under standard conditions or were processed with different sonication powers (low power is 10% of maximum amplitude of a 700 W sonicator; medium is 50% and high is 100%). Nanopieces were imaged under a transmission electron microscope, and their length and width were analyzed with Image J software.

### Example 3.6

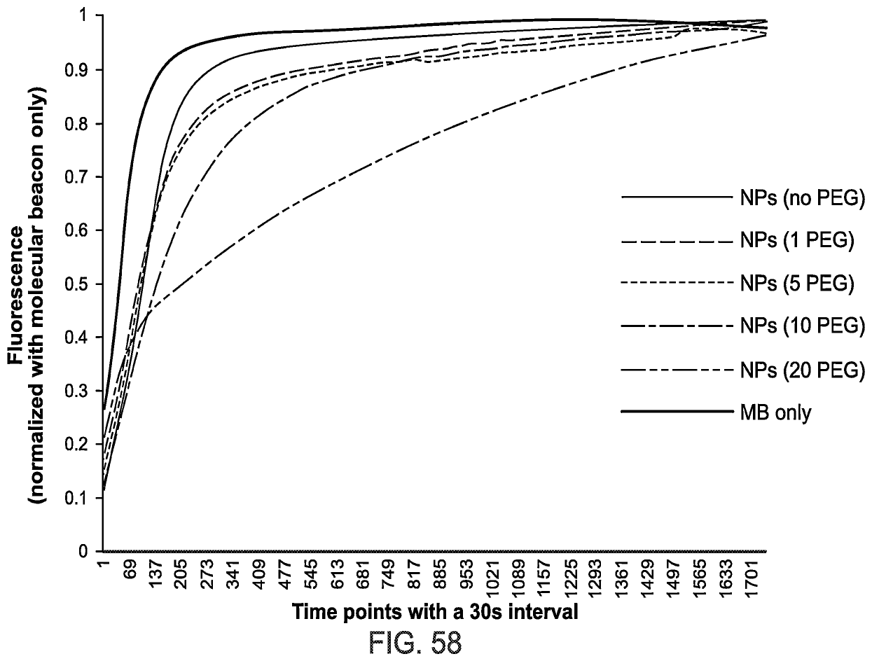

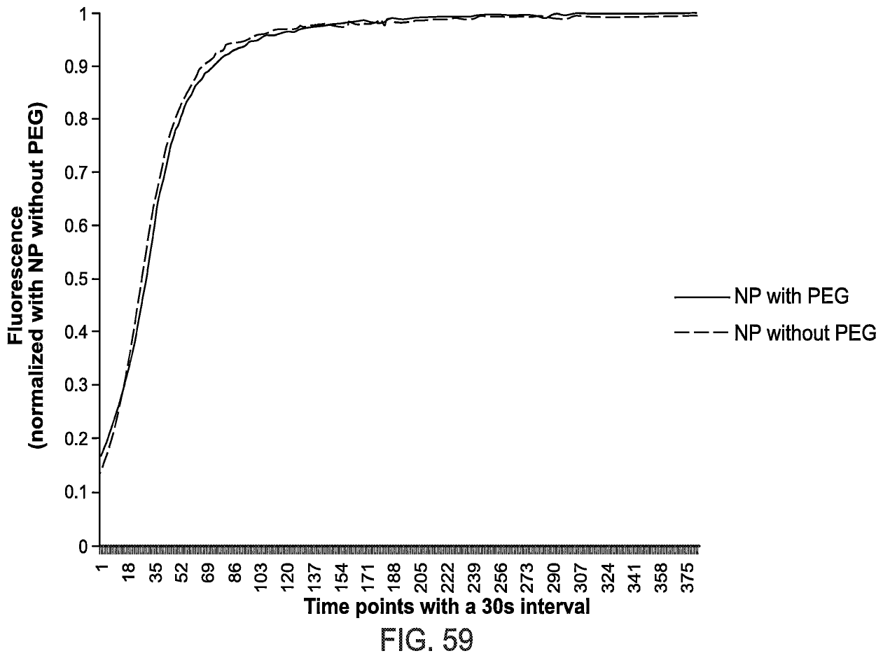

Nanopieces are optionally coated. Coating of Nanopieces with PEG facilitated Nanopieces delivery into tissue matrix, especially in a protein-rich environment, such as in the presence of serum (FIG. 20). Although Nanopieces doubled the half-life of delivery cargos (such as molecular beacon, MB) in serum, a covalent linked PEG coating had a 6-time longer half-life than MB only (FIG. 58). Moreover, non-covalent linked PEG only had marginal difference on Nanopieces in terms of stability in serum (FIG. 59). FIGS. 58-59 shows that molecular beacons delivered with/without Nanopieces were soaked in serum. For PEG coating, PEG (MW 400) was either covalently linked or non-covalently coated on Nanopieces. A fluorescence plate read was determined half-life of MBs.

### Example 3.6

Nanopieces of different sizes and length were prepared using the following procedure:
Step A: Quench before assembly: heating 5 ug RNT in water to 50-99° C. for 10 s-10 mins, then immediately putting it on ice, and mixing with 50 pmol siRNA, then, sonicating for 30 s-2 mins to produce Nanopieces.
Step B: Sonication before assembly: sonicating 5 ug RNT in water to 50-99° C. for 10 s-10 mins, and mixing with 50 pmol siRNA, then, sonicating for 30 s-2 mins to produce Nanopieces.
Step C: Increase ionic strength: mixing 5 ug RNT with 50 pmol siRNA in saline, then, sonicating for 30 s-2 mins to produce Nanopieces.
Step D: Increase sonication time after assembly: mixing 5 ug RNT with 50 pmol siRNA, then, sonicating for 2 mins-10 mins to produce Nanopieces.

### Modification of Parameters:

| | Size of Nanopieces | |
|---|---|---|
| Factors | High/Long | Low/Short |
| Heating temperature for | Small | Large |
| quench | (Avg. length 10 nm~149 nm; Avg. | (Avg. length 150 nm~500 nm; |
| | width diameter 10~29 nm) | Avg. width diameter 10~29 nm) |
| Heating time for quench | Small | Large |
| | (Avg. length 10 nm~149 nm; Avg. | (Avg. length 150 nm~500 nm; |
| | width diameter 10~29 nm) | Avg. width diameter 10~29 nm) |
| Sonication time before | Small | Large |
| assembly | (Avg. length 10 nm~149 nm; Avg. | (Avg. length 150 nm~500 nm; |
| | width diameter 10~29 nm) | Avg. width diameter 10~29 nm) |
| Sonication power before | Small | Large |
| assembly | (Avg. length 10 nm~149 nm; Avg. | (Avg. length 150 nm~500 nm; |
| | width diameter 10~29 nm) | Avg. width diameter 10~29 nm) |
| Sonication time after | Small | Large |
| assembly | (Avg. length 10 nm~149 nm; Avg. | (Avg. length 150 nm~500 nm; |
| | width diameter 10~29 nm) | Avg. width diameter 10~29 nm) |
| Sonication power after | Small | Large |
| assembly | (Avg. length 10 nm~149 nm; Avg. | (Avg. length 150 nm~500 nm; |
| | width diameter 10~29 nm) | Avg. width diameter 10~29 nm) |
| Ionic strength | Vary Large | Small |
| | (Avg. length 150 nm~999 micon; | (Avg. length 10 nm~149 nm; |
| | Avg. width diameter 30~100 nm) | Avg. width diameter 10~29 nm) |

| | Charge of Nanopieces | |
|---|---|---|
| | Strong/High | Weak/Low |
| RNT/RNA ratio | Positive | Negative |
| Negative charge from the | Negative | Positive |
| cargo (such as RNA other | | |
| nucleic acids or proteins) | | |

| Nanopiece | Size | | Surface Charge | |
|---|---|---|---|---|
| properties | Small | Large | Negative | Positive |
| Suitable cells | High and | Low and | Positively | Negatively |
| or tissues | dense | loose | charged or | charged or |
| | extracellular | extracellular | neutral cell | neutral cell |
| | matrix content | matrix | membrane/ | membrane/ |
| | | content | extracellular | extracellular |
| | | | matrix | matrix |

### Example 4

### Surface Charge and Matrix/Tissue Binding

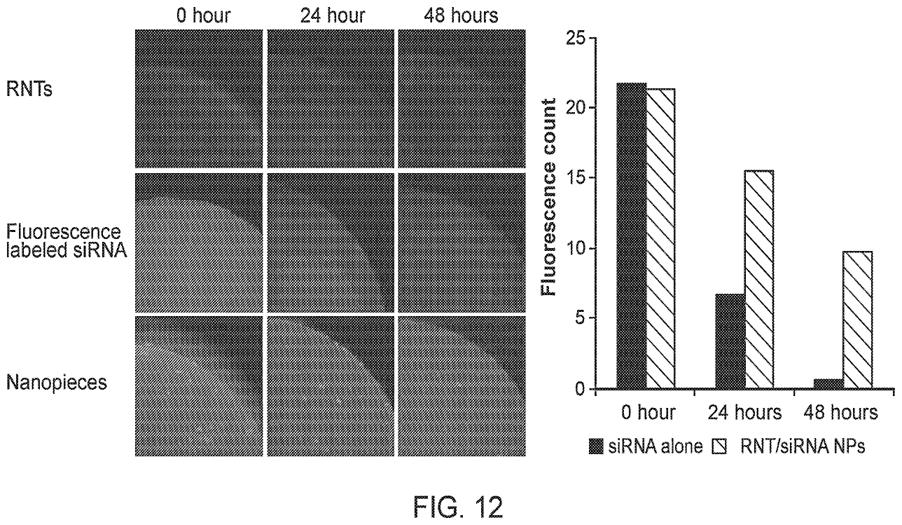

Surface charge of Nanopieces were tuned or customized via controlling RNT/delivery cargo ratio (e.g., RNT/siRNA as an example, FIG. 11). Adjusting 4.4 μg-30 μg RNTs per 0.1 nmol RNA yielded positively charged Nanopieces. These Nanopieces exhibited excellent binding to negatively charged tissue and/or matrix, as shown in FIG. 12; light grey area and spots are the fluorescence signals from siRNA alone or siRNA. Nanopieces with more than 30 ug RNT per 0.1 nmol RNA are also positively charged. Generally, the ratio will not exceed 30 ug per 0.1 nmol RNA.

### Example 4.1

Fluorescence labeled RNA with and without Nanopieces was added onto porcine articular cartilage for 1 h. Then, the cartilage was soaked in HBSS buffer at 3TC. The remaining RNA was analyzed using a fluorescence microscope.

### Example 5

### Trans-Matrix/Tissue Delivery

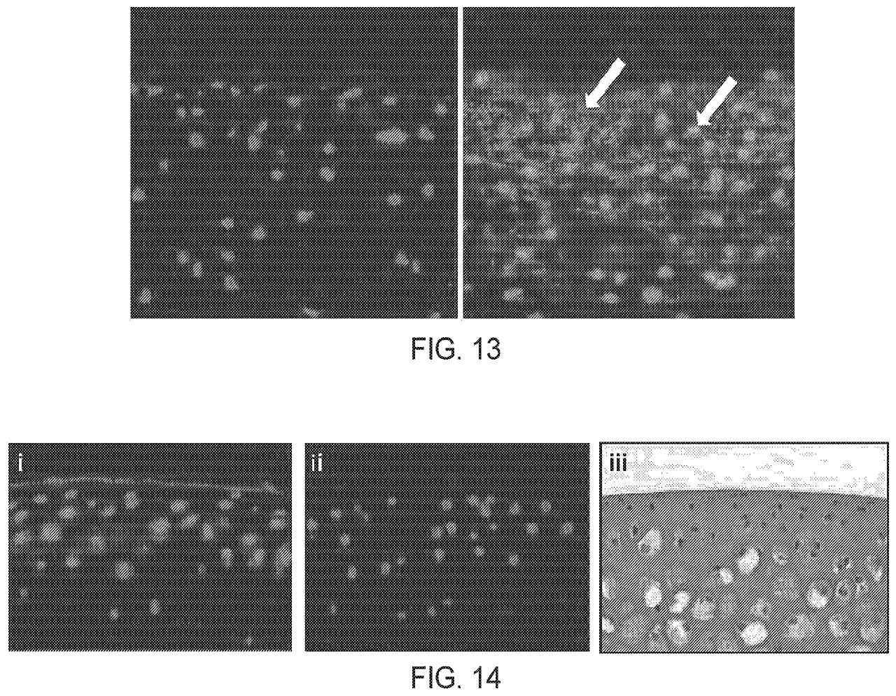

Results showed that processed fluorescence labeled siRNA/RNT Nanopieces successfully penetrated into cartilage (FIG. 13). Moreover, it was further demonstrated that GAPDH molecular beacon/RNT Nanopieces not only penetrate into the tissue matrix but also inside cells (FIGS. 14-16). Effective trans-matrix and/or tissue delivery was demonstrated with a variety of species. Light gray areas within FIG. 14-16 around the cell nucleus are the fluorescence signals from molecular beacons.)

### Example 5.1

Fluorescence labeled RNA was delivered with and without Nanopieces and was soaked with porcine cartilage. After 24 hours, the cartilage was sectioned and the individual sections were observed under a fluorescence microscope (FIG. 13).

### Example 5.2

Fluorescence GAPDH molecular beacon was delivered with and without Nanopieces and soaked with mouse cartilage. After 24 hours, the cartilage was then sectioned and the individual sections were observed under a fluorescence microscope (FIG. 14).

### Example 5.3

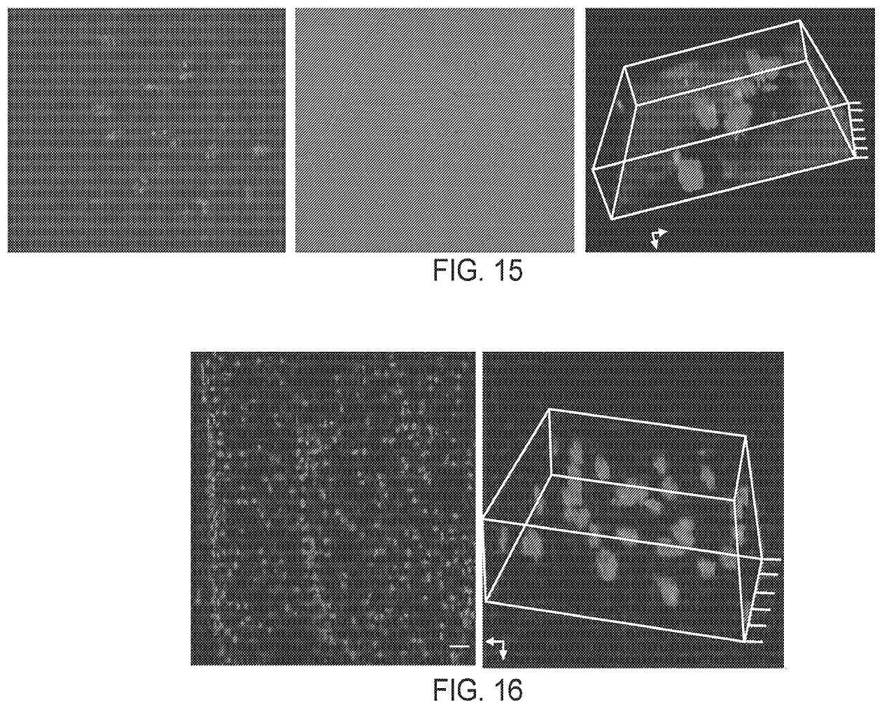

Fluorescence GAPDH molecular beacon was delivered with and without Nanopieces and soaked with human cartilage. After 24 hours, the cartilage was then sectioned and the individual sections were observed under a fluorescence microscope (FIG. 15).

### Example 5.4

Fluorescence GAPDH molecular beacon was delivered with and without Nanopieces and soaked with chicken cartilage. After 24 hours, the cartilage was then sectioned and the individual sections were observed under a fluorescence microscope (FIG. 16).

### Example 5.5

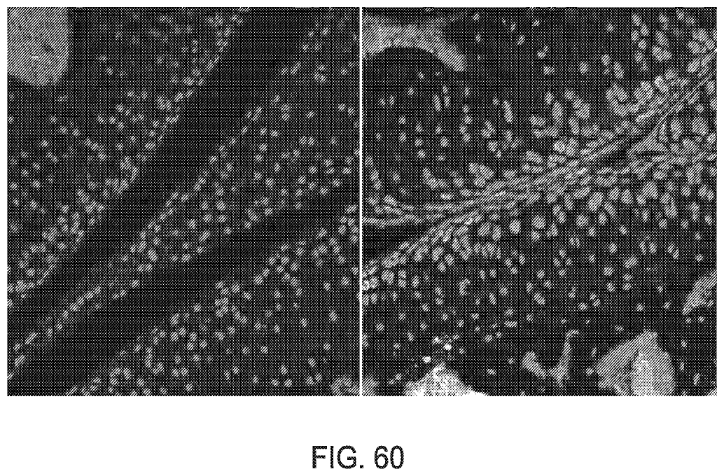

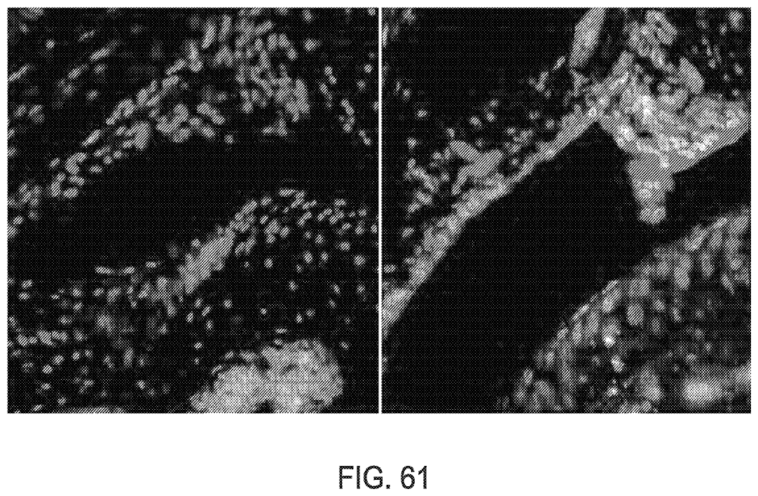

Applications of various types of Nanopieces: Various types of Nanopieces can be used for delivery into different tissues or organs as desired. For example, co-injection of small Nanopieces (Avg. length ˜110 nm, Avg. width ˜20 nm) (SMALL means Avg. length 10 nm˜149 nm; Avg. width diameter 10˜29 nm) to deliver GAPDH MBs with fluorescence and very large Nanopieces (Avg. length ˜250 nm, Avg. width ˜33 nm) (LARGE means Avg. length 150 nm˜999 micon; Avg. width diameter 30-100 nm) to deliver GAPDH MBs also with fluorescence into knee joints of mice were carried out. Small Nanopieces could be delivered into both cartilage and synovium, while large Nanopieces could only be delivered into synovium (FIGS. 60-61). (Bright area/spots around cell nuclei in FIG. 60-61 are the fluorescence signal from molecular beacons delivered via different sizes of Nanopieces.) Therefore, selective delivery into synovium with processed large Nanopieces was achieved.

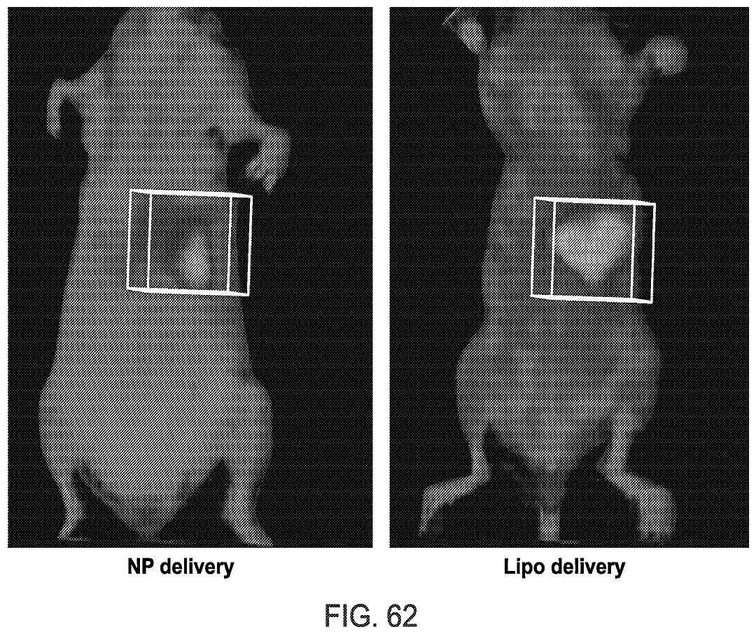

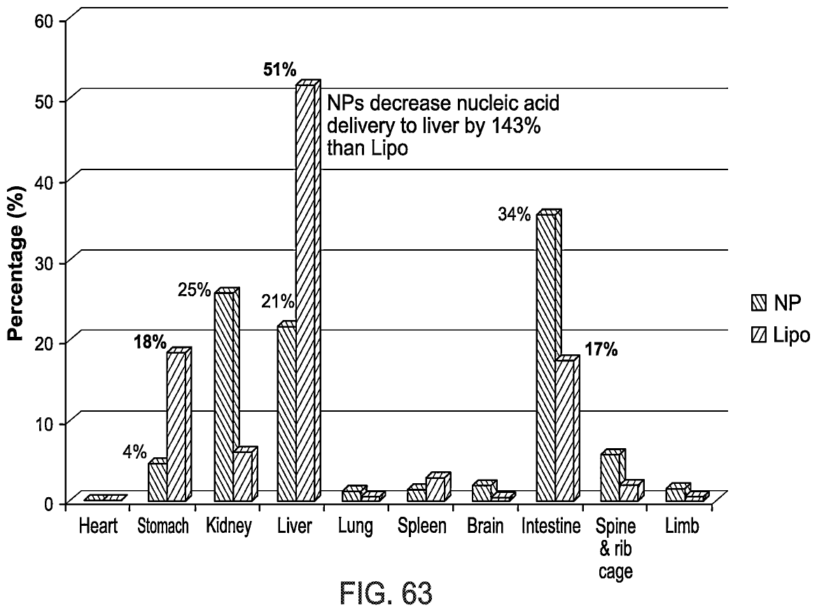

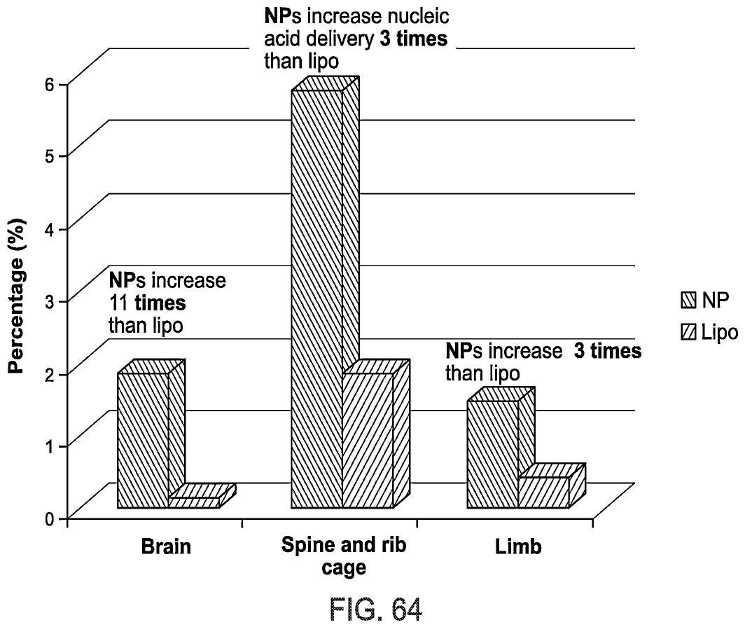

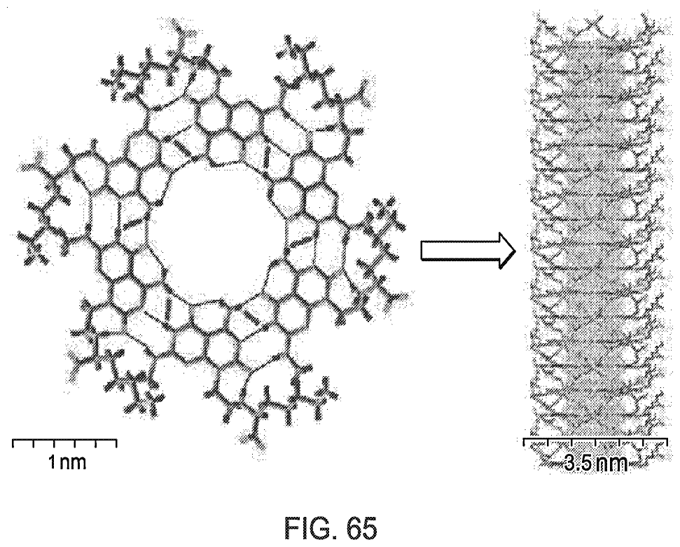

Another example was the use of small Nanopieces. Systemic injection of small Nanopieces into mice was carried out. Compared with conventional lipid delivery vehicles, small Nanopieces were found to be able to increase penetration into tissues and organs with dense matrix, which are difficult to infiltrate (such as brain, rib, spine and limb), as well as decreased liver capture (FIGS. 62-63). FIGS. 60-61 shows fluorescence labeled GAPDH molecular beacon delivered with small Nanopieces and also fluorescence labeled GAPDH molecular beacon delivered with large Nanopieces were co-injected into mouse knee joints, and the fluorescence signal was observed under a fluorescence microscope. FIGS. 62-64 shows Far fluorescence labeled GAPDH molecular beacon delivered with Nanopieces or with lipid particles were injected into mice via resto-orbital injection. After 24 hours, the mice were sacrificed and dissected. The fluorescence signal in each organs or tissue was recorded and via a fluorescence molecular tomography.

### Example 6

### Function

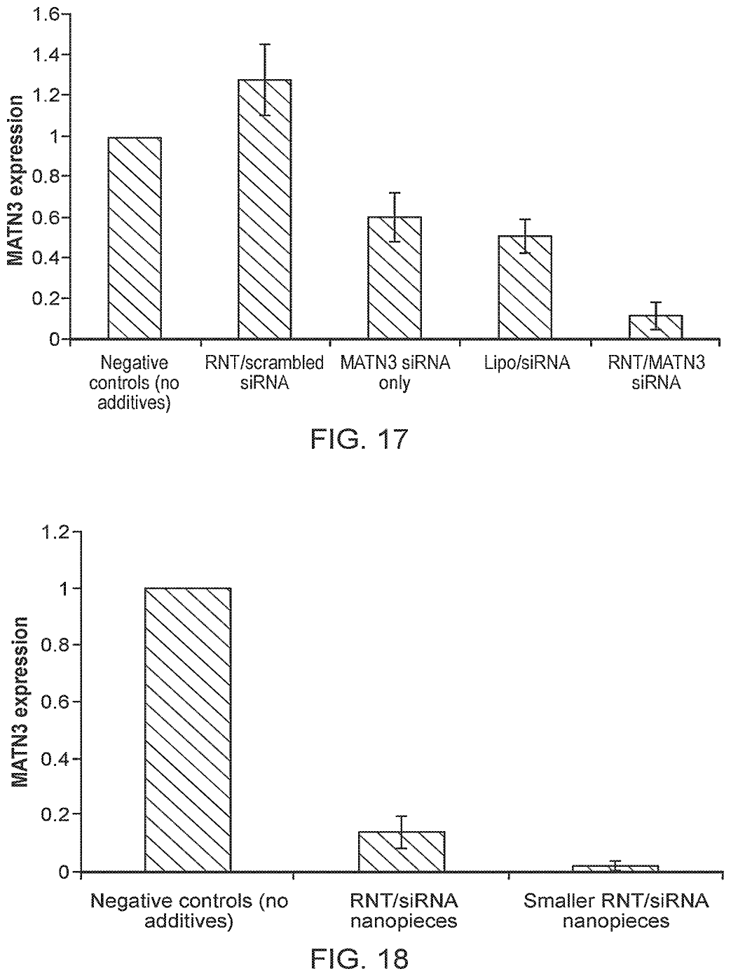

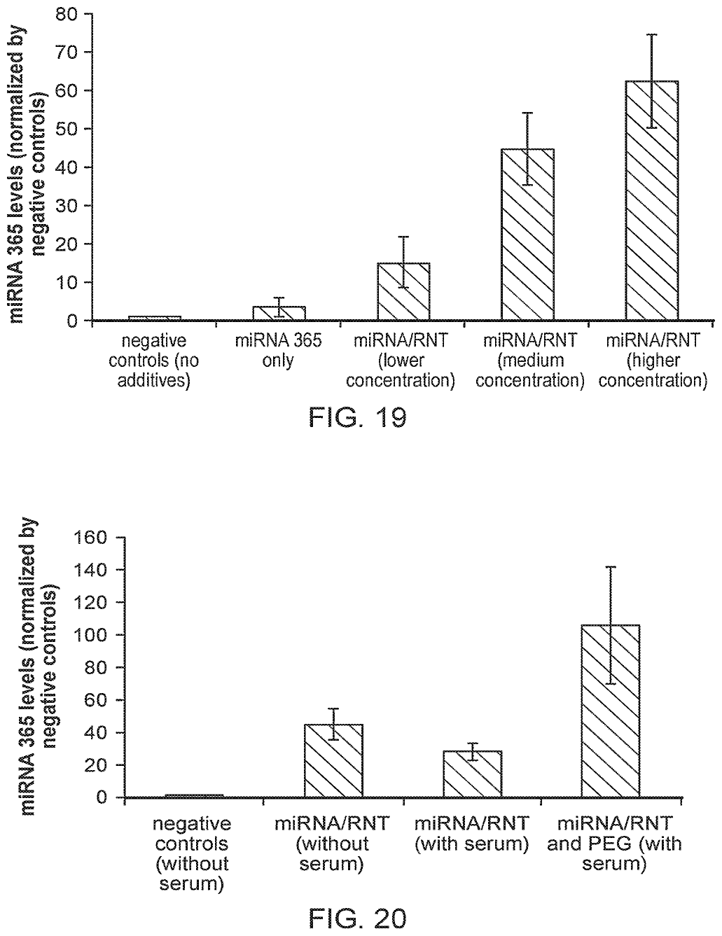

Results showed delivery of Matrilin-3 (MATN3) siRNA/RNT Nanopieces into the mouse cartilage tissue matrix and cells with excellent biological function (FIGS. 17 and 18). Moreover, miRNA-365/RNT Nanopieces were functional, when delivered into human cartilage tissue matrix and cells (FIG. 19). The smaller processed Nanopieces resulted in higher Nanopiece delivery efficacy.

### Example 6.1

MATN-3 siRNA was delivered with and without Nanopieces or Lipofectamine 2000 and soaked with mouse cartilage. The MATN-3 gene expression was determined via real time RT-PCR (FIG. 17).

### Example 6.2

MATN-3 siRNA was delivered with unprocessed or processed Nanopieces and was soaked with mouse cartilage. The MATN-3 gene expression was determined via real time RT-PCR (FIG. 18).

### Example 6.3

Various doses of miR-365 (0.1, 0.5 and 1.0 nmol) were delivered with Nanopieces and were soaked with human cartilage. The miR-365 expression was determined via real time RT-PCR (FIG. 19).

### Example 7

### Compositions

FIG. 20 shows that a composite of PEG increases Nanopiece delivery efficiency in a protein-rich environment (such as serum).

### Example 8

### In Vivo Delivery

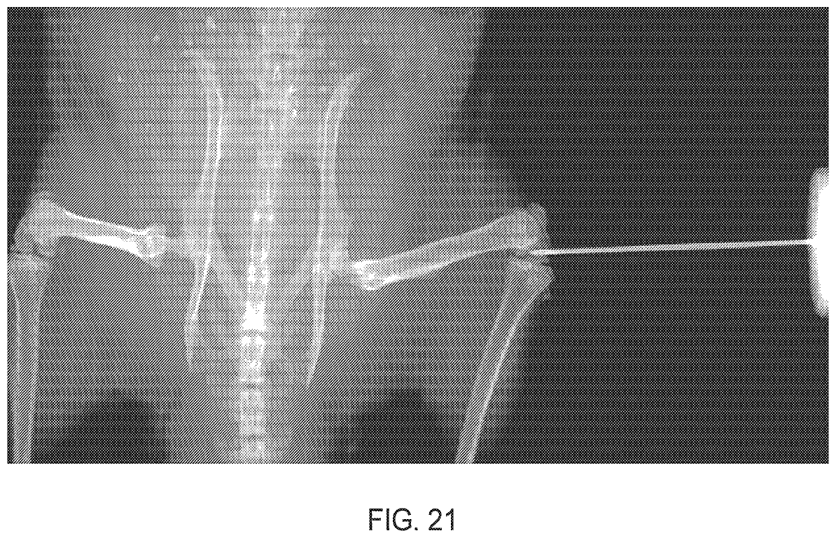

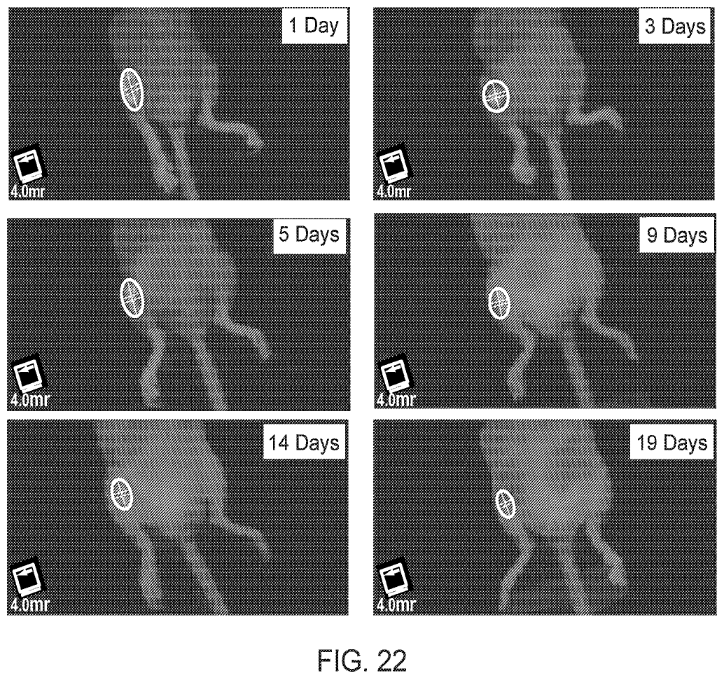

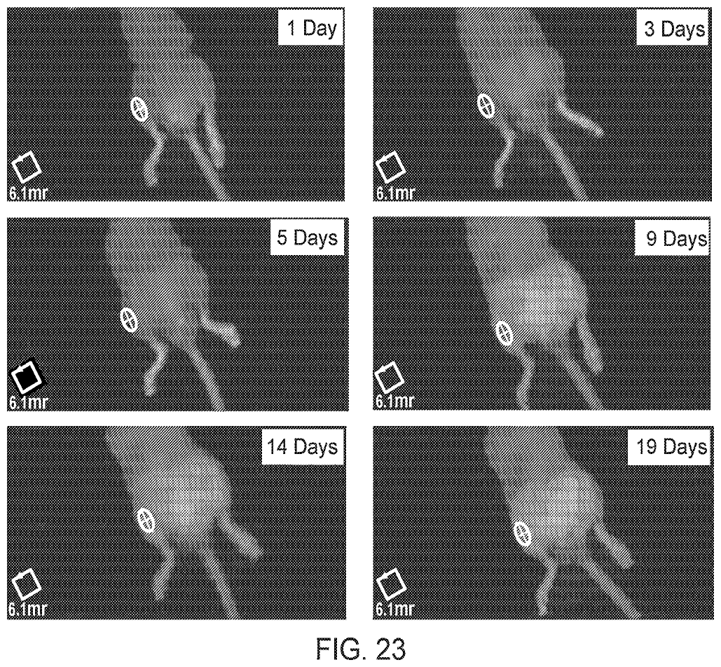

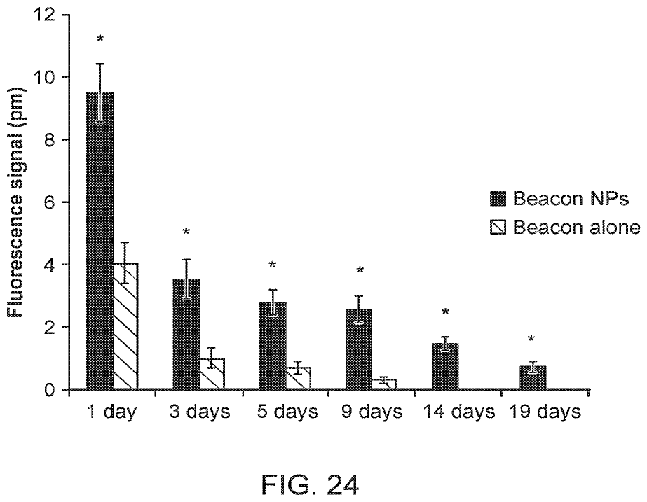

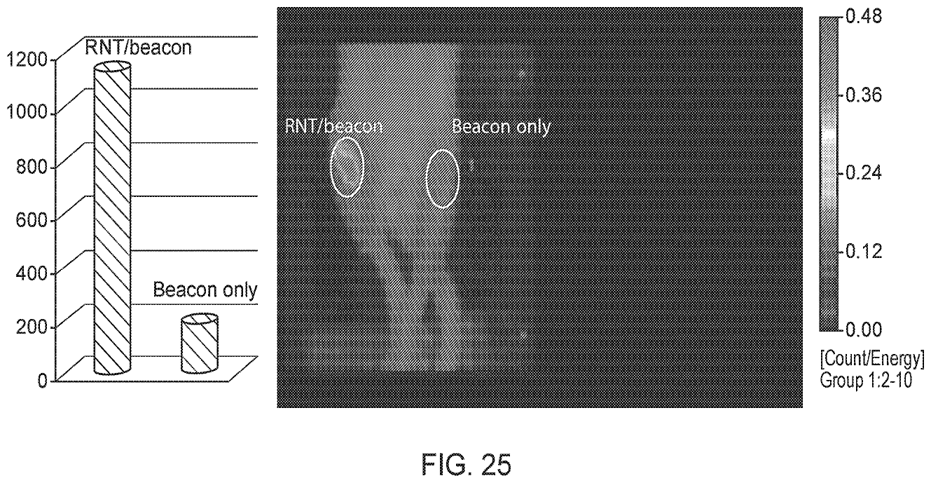

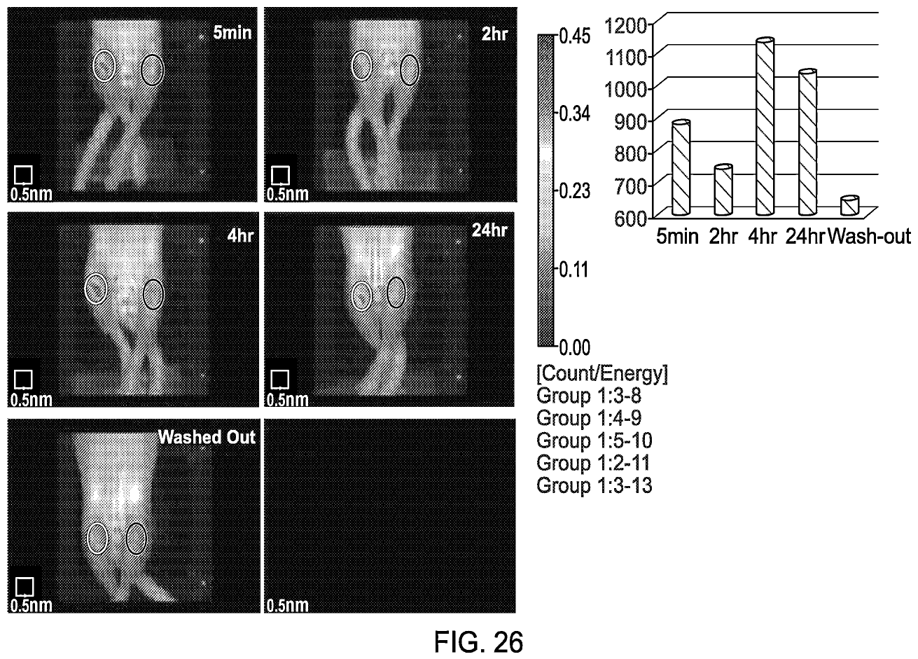

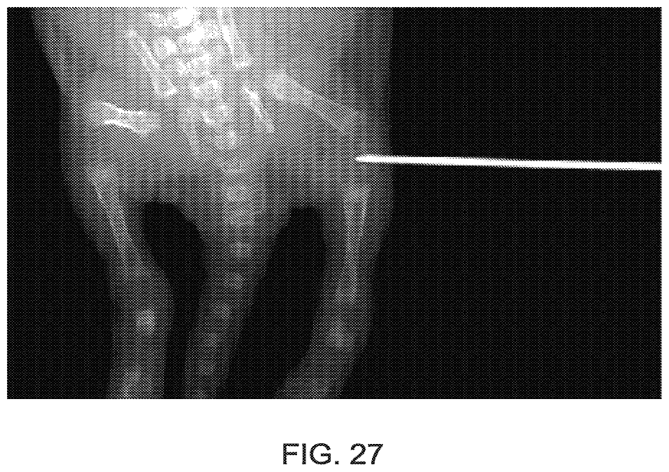

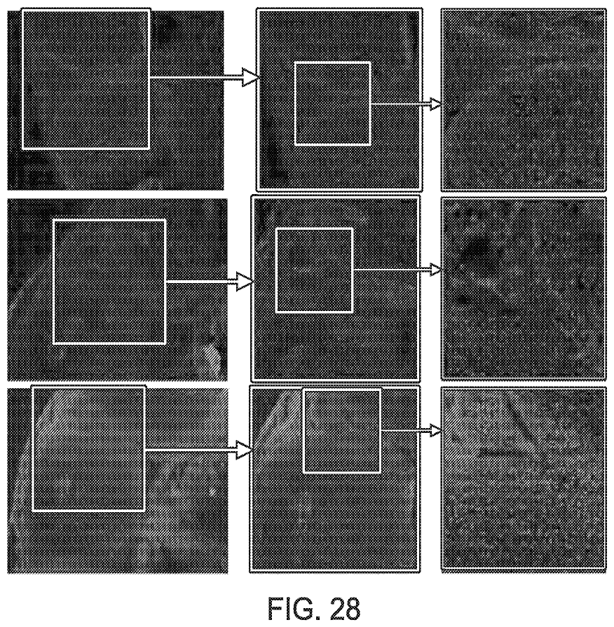

FIGS. 21 and 27 show injection of Nanopieces into an articulating joint. Injection of GAPDH molecular beacon/RNT Nanopieces into knee joints of a mouse (FIG. 21) resulted in a significant fluorescence signal compared with beacon only (in the absence of RNT Nanopieces). The signal lasted more than 2 weeks in the knees (FIGS. 22-24). In rats, a significant fluorescence signal was also obtained by injecting GAPDH molecular beacon/RNT Nanopieces into knee joints. The fluorescence signal was robust after washing out the adhered fluorescence molecules on the articular surface (FIGS. 25-26). Matrilin-3 siRNA Nanopieces were injected into knees of baby one-week-old mice and was found to be functional. Histology slides of cartilage sections confirmed the successful delivery of the Nanopieces (FIG. 28; light grey areas around the cell nuclei illustrate the fluorescence signal from molecular beacons. Effective in vivo trans-matrix/tissue delivery of processed Nanopieces (Nanopieces) was demonstrated in these experiments.

### Example 8.1

Fluorescence labeled GAPDH molecular beacon was delivered with and without Nanopieces and injected into mouse knee joints. The fluorescence signal was recorded via fluorescence molecular tomography (FIGS. 22-24).

### Example 8.2

Fluorescence labeled GAPDH molecular beacon was delivered with and without Nanopieces and injected into rat knee joints. The fluorescence signal was recorded via fluorescence molecular tomography (FIGS. 25-26).

### Example 8.3

Fluorescence labeled GAPDH molecular beacon was delivered with and without Nanopieces and injected into baby mouse knee joints. The mouse was sacrificed and knee joint was sectioned for observation under a fluorescence microscope (FIGS. 27-28; light grey areas around the nuclei in FIG. 28 illustrate the fluorescence signal from molecular beacons.

### Example 9

### Diagnostics

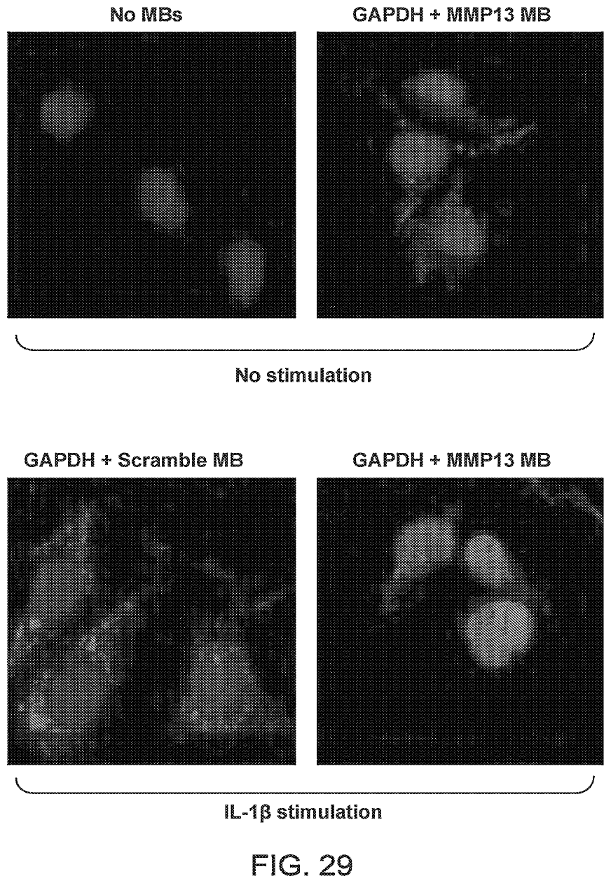

To detect OA progression, MMP-13 was selected as a target gene. MMP-13 molecular beacon was designed and its function validated in vitro. As shown in FIG. 29, MMP-13 molecular beacon was delivered by methods described herein and found to emit fluorescence in chondrocytes after stimulation. Light areas shown in in FIG. 29 illustrate the fluorescence signal from molecular beacons. The MMP-13 molecular beacon was prepared according to the following procedure:
Step one: Pre-heat RNT nanotubes solution, then quench it by placing tube on ice.
Step two: Sonicate RNT nanotubes solution.
Step three: Dilute MMP-13 molecular beacon or IL-1 beta receptor siRNA in water, then mix with RNT nanotubes solution in a certain ratio (50 pmol siRNA or 100 pmol molecular beacon to 5 ug RNT), then vertex well.
Step four: Sonicate the mixture described in Step three, then spin all liquid down. MMP-13 molecular beacon or IL-1 beta receptor Nanopieces was assembled after Step four.
   *Standard preparation only includes Step three and Step four. Joint preparation includes all steps.

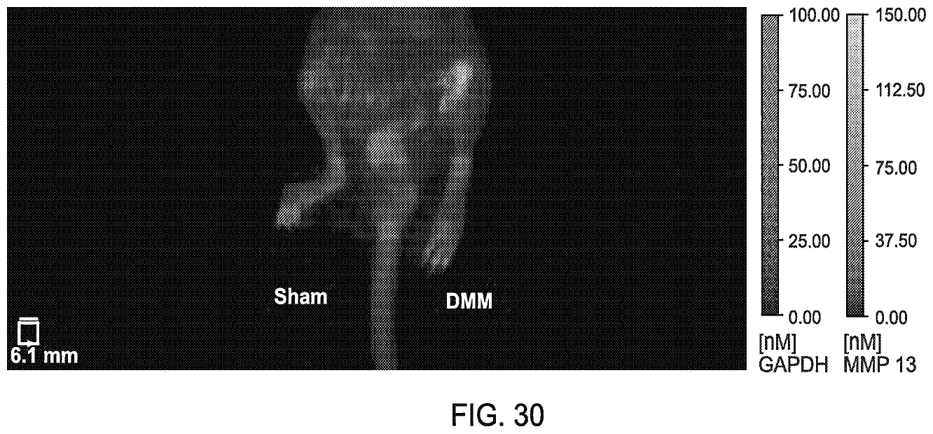

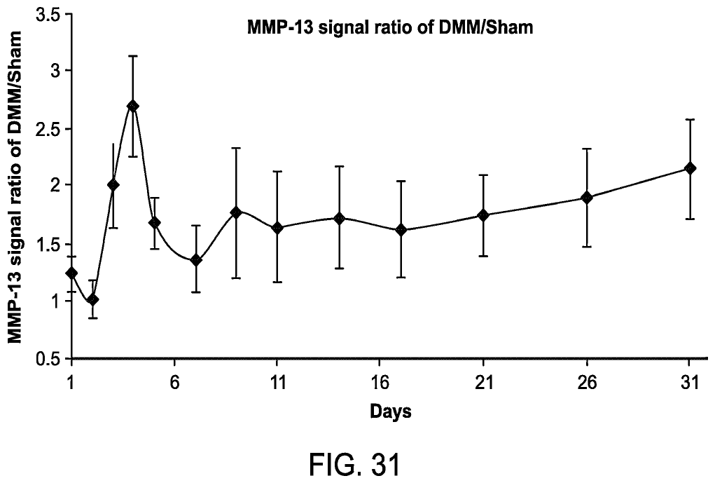

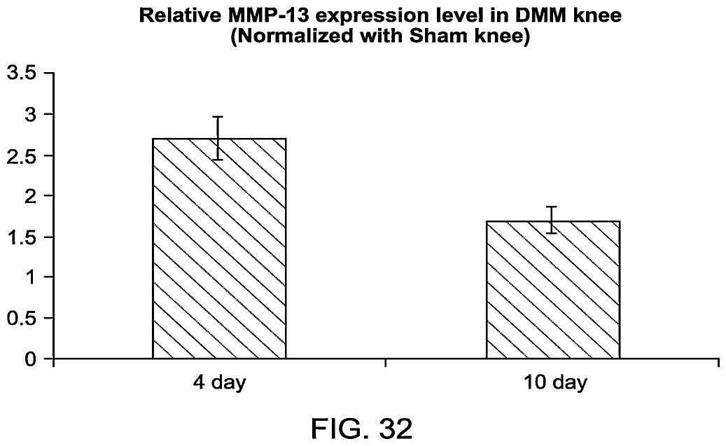

For in vivo diagnosis, the medial meniscus (DMM) was destabilized to induce OA on one knee of the mice, whereas on the other knee a sham surgery was performed. Right after surgery, MMP-13 molecular beacon was delivered for target gene detection together with a non-targeting scrambled molecular beacon as a non-specific signal serving as a negative control. In addition a GAPDH molecular beacon for an internal house-keeping gene control was also administered. After 4 days, the knee with OA induction, showed a significantly stronger signal than the sham knee (FIG. 30). Moreover, using such a real-time, in-situ, non-invasive diagnosis approach, the signals between DMM and sham were quantitatively compared in a time-depend curve (FIG. 31). Methods were provided to continuously monitor a specific gene expression during OA progression in living animals. Moreover, animals were sacrificed at day 4 and day 11 to determine their MMP-13 expression level via real time RT-PCR. Results showed that the non-invasive diagnostic technology described herein accurately detected gene expression level compared with PCR (FIG. 32).

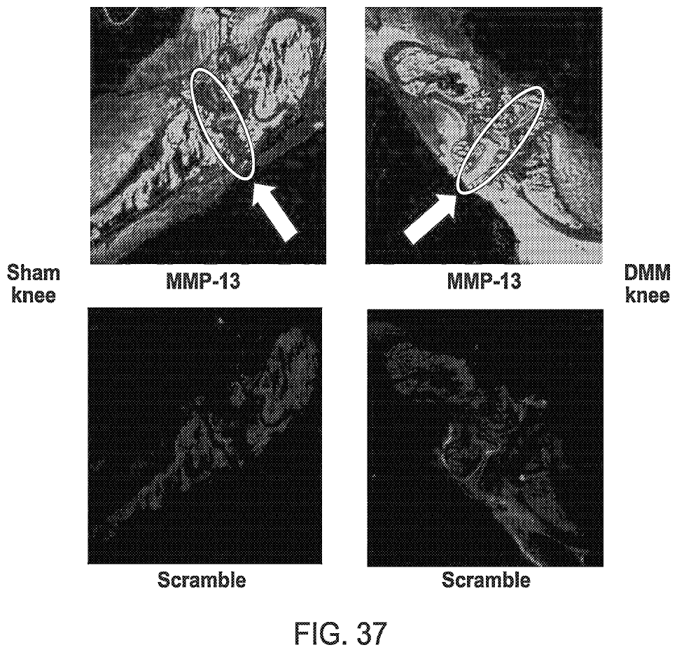

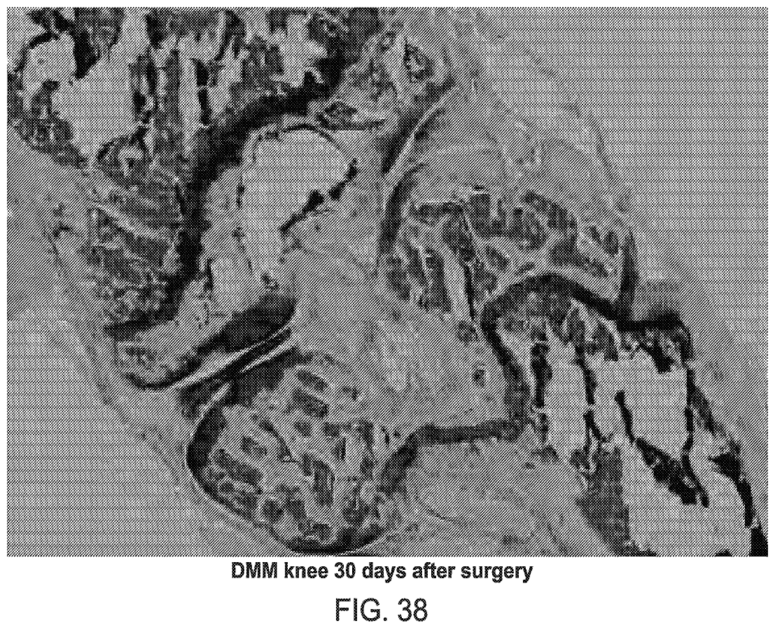

Fluorescence and histology analysis showed that the damaged articular cartilage surface was the area emitting fluorescence signal from MMP-13 molecular beacon (FIGS. 37-38). In FIG. 37, ARROWs indicate the fluorescence signal as a result from MMP-13 molecular beacon. In FIG. 38, the dark grey color in articular cartilage was aggrecan staining. DMM surgery resulted in loss of aggrecan staining and damage to articular cartilage.

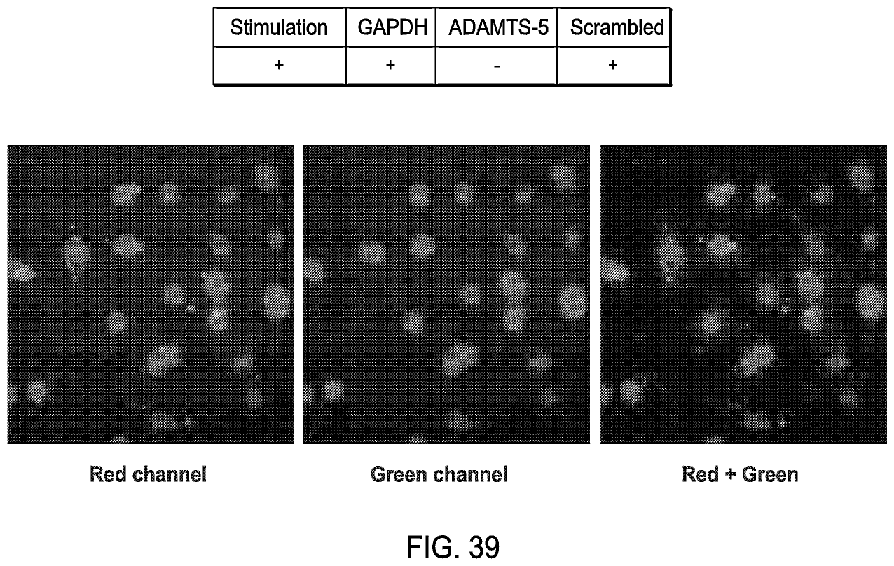

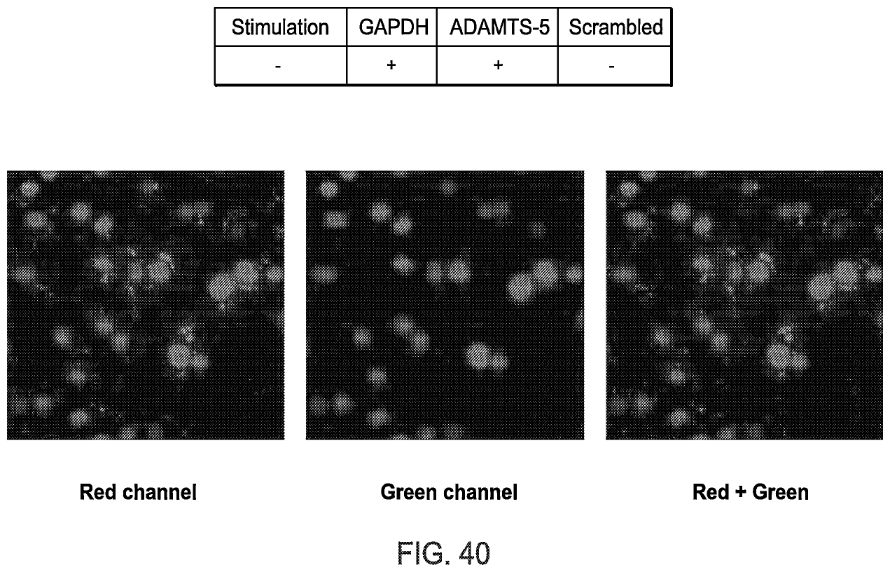

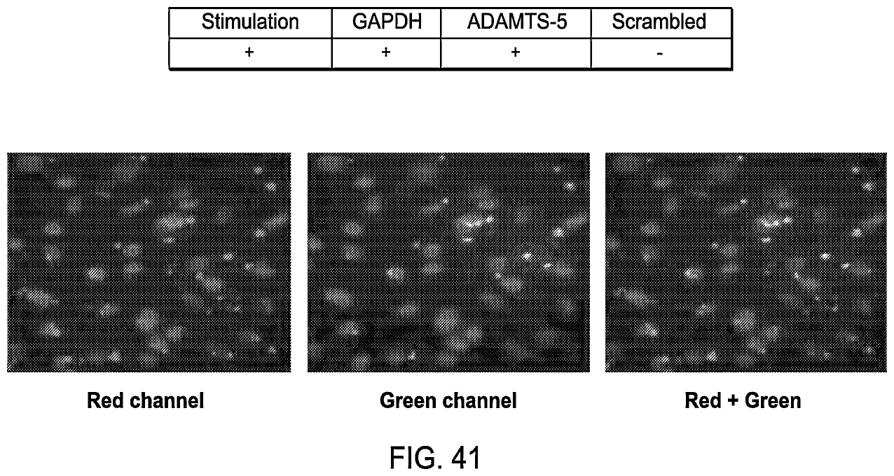

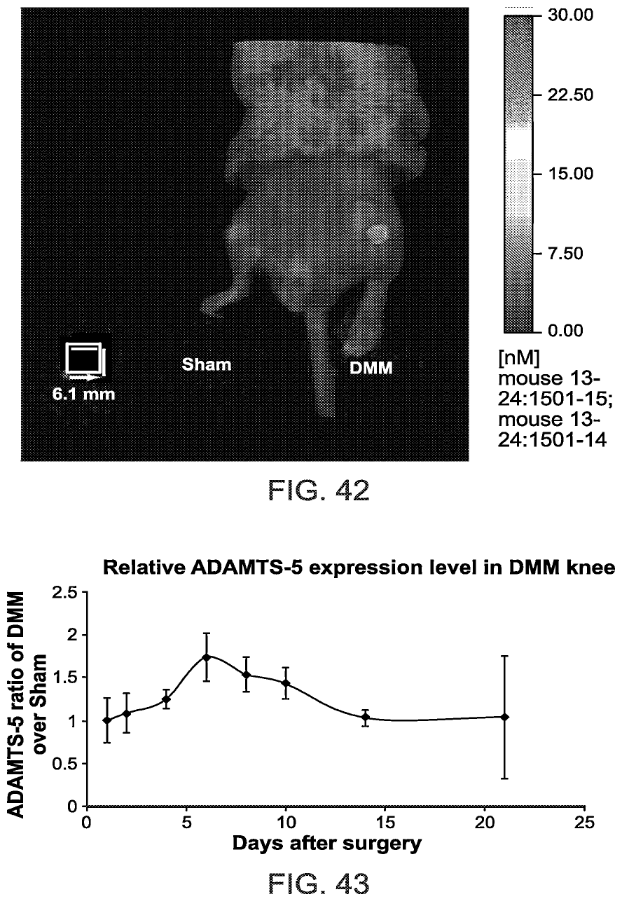

In addition to MMP-13, ADAMTS-5 molecular beacon for OA diagnosis was also shown. Again, the ability of this molecular beacon to detect ADAMTS-5 gene expression in vitro was demonstrated (FIGS. 39-41; light grey areas around the cell nuclei in FIG. 39-41 are the fluorescence signal from molecular beacons. RED channel showed signal from GAPDH beacons; while GREEN channel showed signal from ADAMTS-5 or Scrambled beacons. The up-regulation pattern of ADAMTS-5 during OA development was also shown (FIGS. 42-43).

These data indicate that the methods are useful for accurate and specific gene expression detection, thereby permitting reliable diagnosis in a real-time, in-situ and in a non-invasive manner in living animals.

### Example 9.1

Fluorescence labeled GAPDH molecular beacon and fluorescence labeled MMP-13 molecular beacon or fluorescence labeled scrambled molecular beacon delivered with Nanopieces was added into chondrocytes under standard cell culture conditions or stimulated with 10 ng/mL IL-10 (FIG. 29).

Using an established method (Tyagi et al Nat. Biotech, 1998, 16:49-53), MBs were designed to target mouse MMP-13 or GAPDH mRNA with a fluorophore/quench pair. Scramble sequence MB (Scramble) was verified to not bind with any mouse mRNA via BLAST. In vitro delivery and validation: MBs were delivered into chondrocytes by Nanopieces. Specifically, after stimulation with IL-10 for 24 hours, chondrocytes were co-transfected GAPDH and scramble MBs or GAPDH and MMP-13 MBs via Nanopieces. Real time RT-PCR and fluorescence microscopy were used to verify the stimulation of MMP-13 expression and the successful fluorescence signal resulted from MMP-13 MB.

To test the efficacy of mRNA detection in chondrocytes using MBs delivered by Nanopieces, primary mouse chondrocytes were transfected with MBs either with or without IL-1β treatment. Before IL-1β treatment, the housekeeping GAPDH MB was detected while the MMP-13 MB was not (FIG. 29, left panels). In contrast, after IL-1β treatment, both GAPDH MB and MMP-13 MB were detected, indicating the induction of MMP-13 mRNA levels by IL-1β (FIG. 29, right panels). Realtime rtPCR showed that MMP-13 mRNA level was up-regulated by about 10 times upon IL-10 stimulation. In contrast, Scramble MB transfection did not show any fluorescence, indicating that the fluorescence of MMP-13 MB was not due to non-specific degradation.

### Example 9.2

Fluorescence labeled GAPDH, MMP-13 and Scrambled molecular beacon delivered with Nanopieces was injected into mouse knee joints after destabilization of medial meniscus (DMM) surgery or Sham surgery, and then the fluorescence signal was recorded and analyzed via a fluorescence molecular tomography (FIGS. 30-31). DMM or sham surgeries were performed on 10-week-old 129SVE male mice to induce osteoarthritis. One week after surgery, MMP-13 and scramble MBs with different fluorophores delivered by Nanopieces were injected into knee joints of mice. Small animal fluorescence molecular tomography (FMT) was used to determine the fluorescence signal that resulted from MMP-13 expression in the live animals for 3 weeks. The Scramble MB showed low fluorescence in both DMM and Sham surgery knee joints. After subtracting Scramble MB basal level signals, MMP-13 MB real signal was about 40 times stronger in the DMM leg than the sham leg (FIGS. 50, and 54-55). Such MMP-13 MB signals persisted, even for 3 weeks after injection of MB s.

### Example 9.3

Mouse knee joint cartilage was isolated 4 days or 10 days after DMM or Sham surgery, and MMP-13 expression was determined via real time RT-PCR (FIG. 32).

### Example 9.4

Fluorescence labeled MMP-13 molecular beacon and Scrambled molecular beacon delivered with Nanopieces was injected into mouse knee joints after DMM or Sham surgery. After 30 days, the animals were sacrificed and their knee joints were sectioned for histology and fluorescence scan (FIGS. 37-38).

### Example 9.5

Fluorescence labeled GAPDH molecular beacon, fluorescence labeled ADAMTS-5 molecular beacon or fluorescence labeled Scrambled molecular beacon delivered with Nanopieces was added into chondrocytes under standard cell culture conditions or stimulated with 10 ng/mL IL-1α and 10 μM retinoic acid (FIGS. 39-41).

### Example 9.6

Fluorescence labeled GAPDH, ADAMTS-5 and Scrambled molecular beacon delivered with Nanopieces was injected into mouse knee joints after DMM or Sham surgery, and then the fluorescence signal was recorded and analyzed via a fluorescence molecular tomography (FIGS. 42-43). FIG. 42 shows a stronger fluorescence signal resulting from ADAMTS-5 molecular beacon in DMM surgery leg than Sham leg. FIG. 43 shows the pattern of ADAMTS-5 expression after surgery.

### Example 10

### Therapeutics

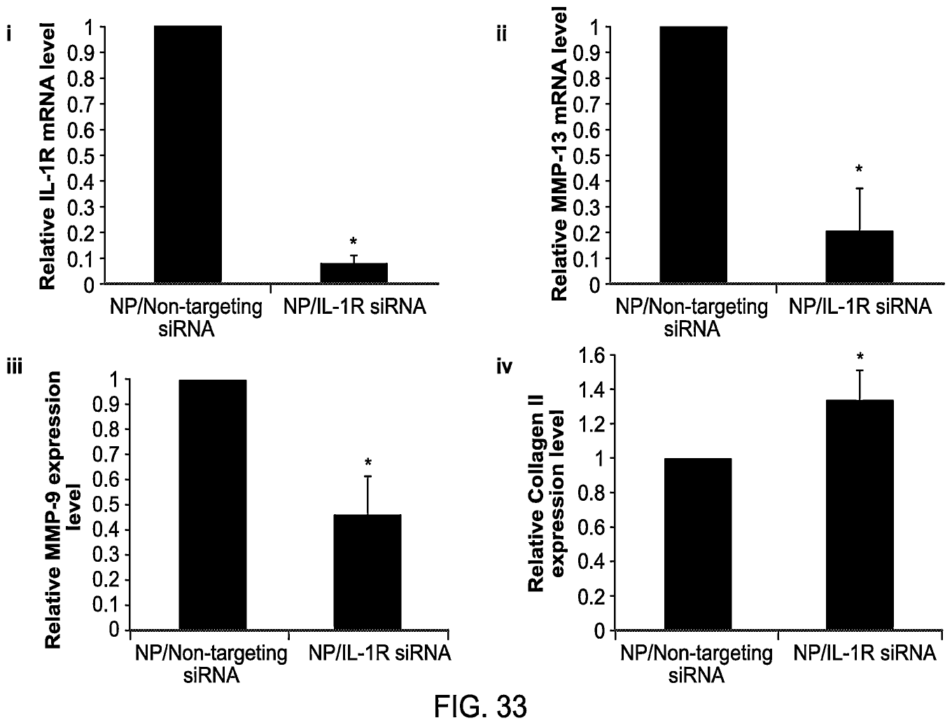

IL-1 receptor (IL-1R) siRNA/Nanopieces were injected into one knee of mice and non-targeting scrambled siRNA/Nanopiece was injected into the other knee. Cartilage degeneration was stimulated with catabolic cytokine (such as IL-1β) in both knees mimicking an inflammation environment during arthritis. Successful knock down of IL-1R in chondrocytes in mouse cartilage was observed with Nanopiece delivery of IL-1R siRNA in vivo (FIG. 33). Moreover, cartilage degeneration genes (such as MMP-13 and MMP-9, FIG. 33) were down-regulated and cartilage anabolic genes (such as Col II, FIG. 33) were up-regulated.

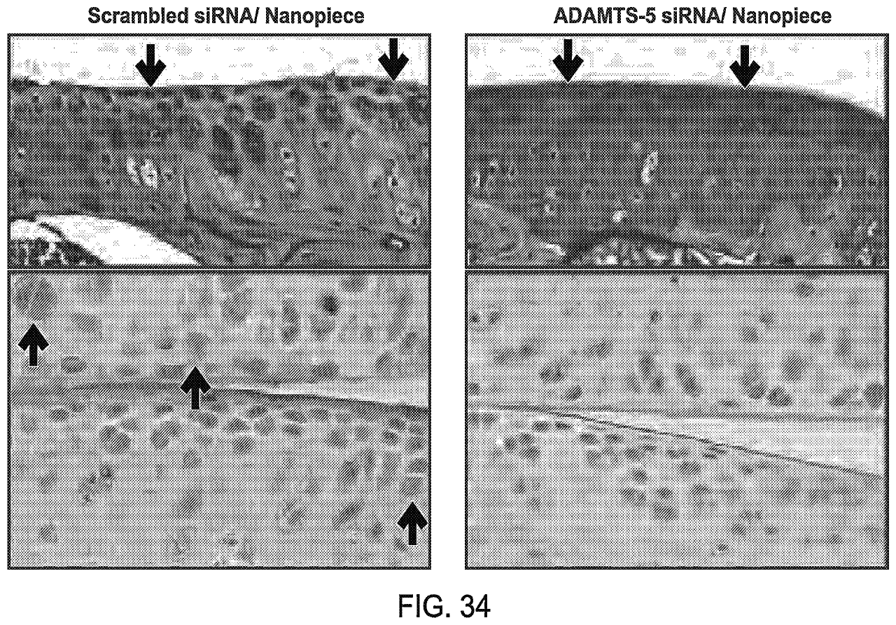

Nanopieces were used to deliver ADAMTS-5 siRNA into knee joints of mice that had been treated with cytokines (IL-1α and retinoic acid). Results showed that cartilage degeneration and aggrecan cleavage was significantly inhibited after ADAMTS-5 siRNA treatment (FIG. 34). In the top two panels, the dark grey color in articular cartilage was aggrecan staining. Without ADAMTS-5 siRNA treatment, aggrecan staining is weaker than the treatment group, indicating loss of aggrecan. In the bottom two panels, dark staining around the cell nuclei was epitope staining from aggrecan cleavage. Without ADAMTS-5 siRNA treatment, the staining is stronger than the treatment group, indicating cleavage of aggrecan.

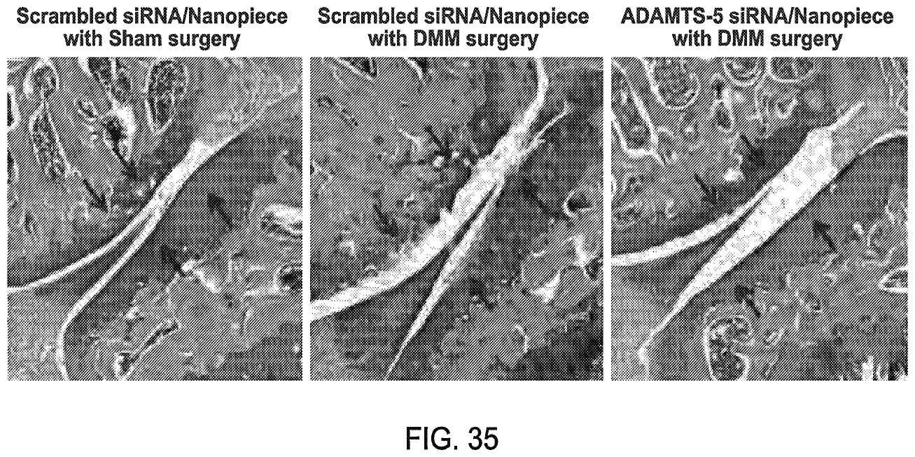

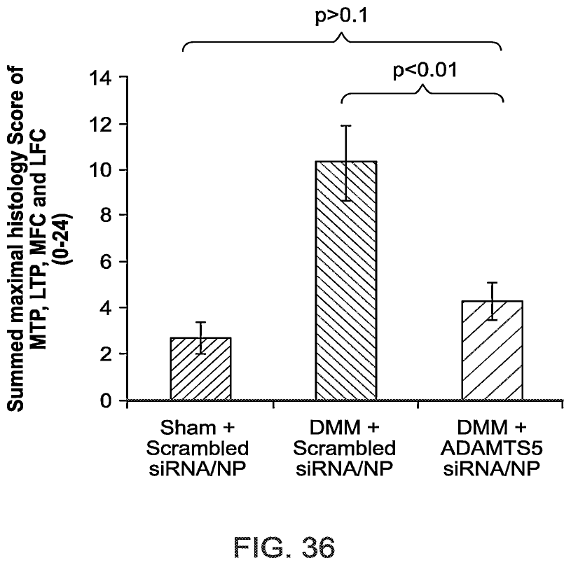

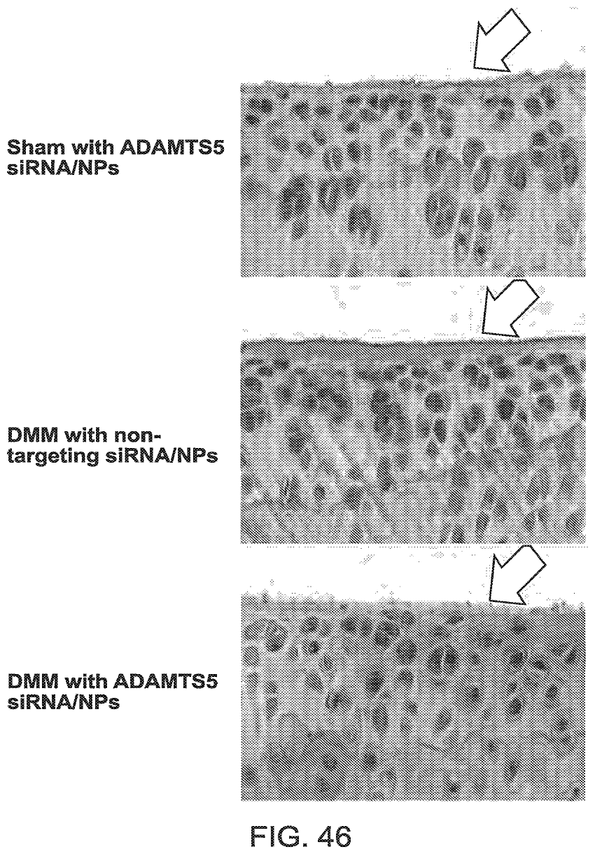

To mimic osteoarthritis progression, DMM surgery on knee joints of mice was conducted. Osteoarthritis progression was shown to be prevented or slowed with Nanopiece delivery of ADAMTS-5 siRNA (FIGS. 35 and 36). In FIG. 35, the dark grey color in articular cartilage was aggrecan staining. A RROWs point out loss of aggrecan staining or damage to articular cartilage in the groups without ADAMTS-5 siRNA treatment; while with treatment, there was very little loss of aggrecan or damage to articular cartilage. Also, immunohisology results showed that aggrecan cleavage was inhibited with delivery of ADAMTS-5 siRNA (FIG. 46). In FIG. 46, dark staining around cell nuclei was epitope staining from aggrecan cleavage. Without ADAMTS-5 siRNA treatment, the staining is stronger than the treatment group, indicating cleavage of aggrecan.

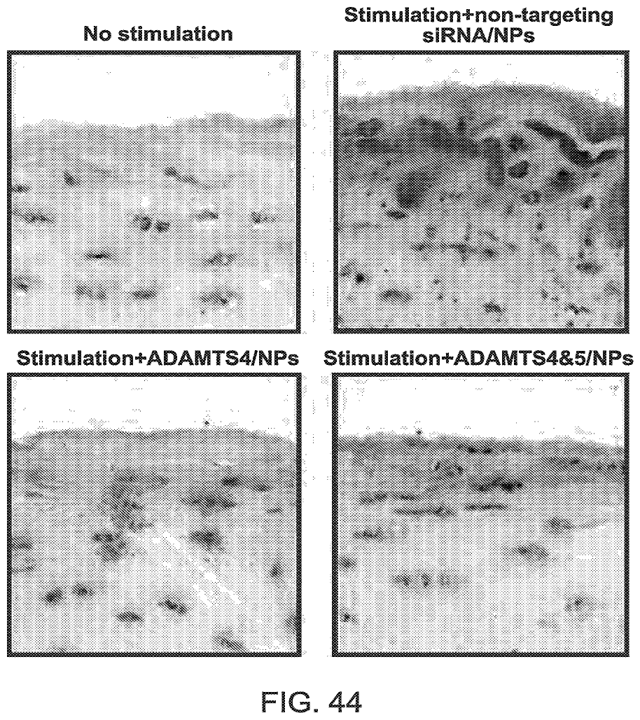

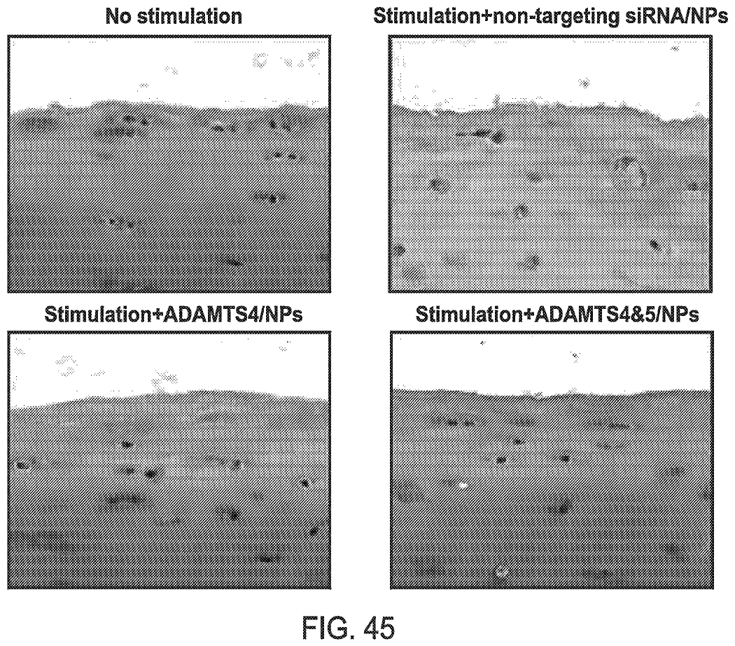

In addition, ADAMTS-5 siRNA was delivered via Nanopieces to human cartilage ex vivo. Protection of human cartilage from cytokine-induced cartilage degradation was demonstrated (FIGS. 44-45). In FIG. 44, dark staining around cell nuclei was epitope staining from aggrecan cleavage. Without ADAMTS-4 or 5 siRNA treatment, the staining is stronger than the treatment group, indicating cleavage of aggrecan. In FIG. 45, dark color in articular cartilage was aggrecan staining. Without ADAMTS-4 or 5 siRNA treatment, aggrecan staining is weaker than the treatment group, indicating loss of aggrecan.

These data indicate that the methods are useful to prevent and/or inhibit cartilage degeneration and arthritis progression.

### Example 11

### Synthesis

### Example 11.1

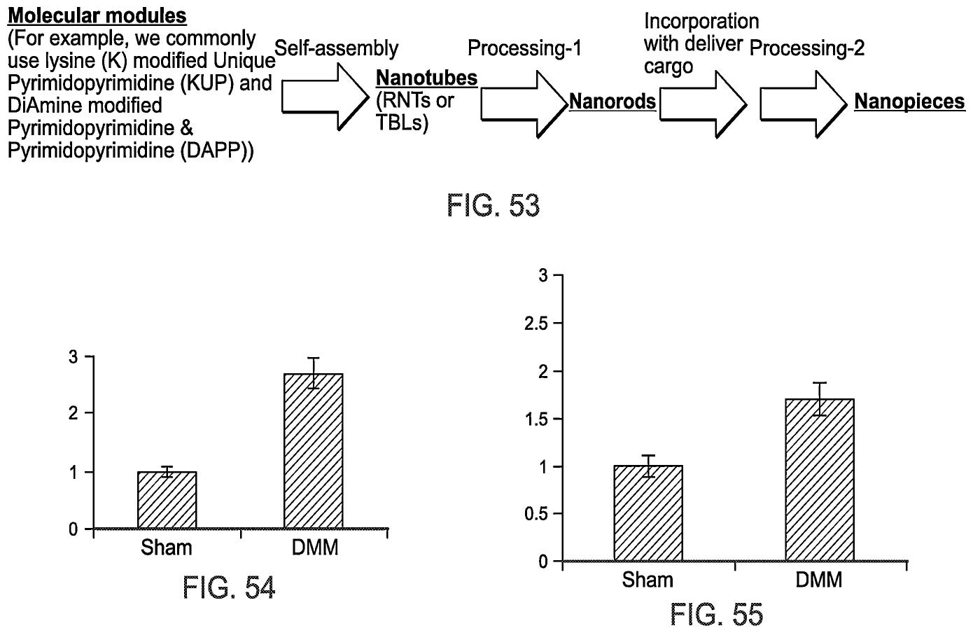

RNTs and TBLs to form Nanopieces are made by first synthesizing a module [(e.g., compound of Formula I or compound of Formula II, respectively]. Nanotubes (RNTs or TBLs) are then processed (Processing-1, Processing-2) to make nanorods and Nanopieces, respectively (see, e.g., FIG. 53). A module for making a Nanopiece was synthesized according to methods described in U.S. Pat. No. 6,696,565 and subsequently purified prior to using the same in the preparation of functional Nanopieces. Liquid chromatography purification was used to purify the synthetic products derived from Formula I and/or Formula II to ensure the success of forming functional and low toxic Nanopieces. In liquid chromatography, trifluoroacetic acid (TFA) is usually applied to keep an acidic eluent environment. Due to known toxicity of TFA or fluoride residual, which made isolated materials undesirable for preclinical and clinical studies, a modification to include hydrochloric acid (HCl) or phosphoric acid during the purification process was developed as an alternative TFA.

Liquid chromatography was performed on C₁₈ reverse-phase column, and Agilent 1260 Infinity Quaternary HPLC System was used. One example of gradient used in isolation is shown below:

| Time | 0 min | 10 min | 15 min |
|---|---|---|---|
| Percentage of Solvent A | 90 | 65 | 0 |
| Percentage of Solvent B | 0 | 25 | 90 |
| Percentage of Solvent C | 10 | 10 | 10 |

*Solvent A is H₂O, Solvent B is 100% acetonitrile, and Solvent C is 0.05N hydrochloric acid.

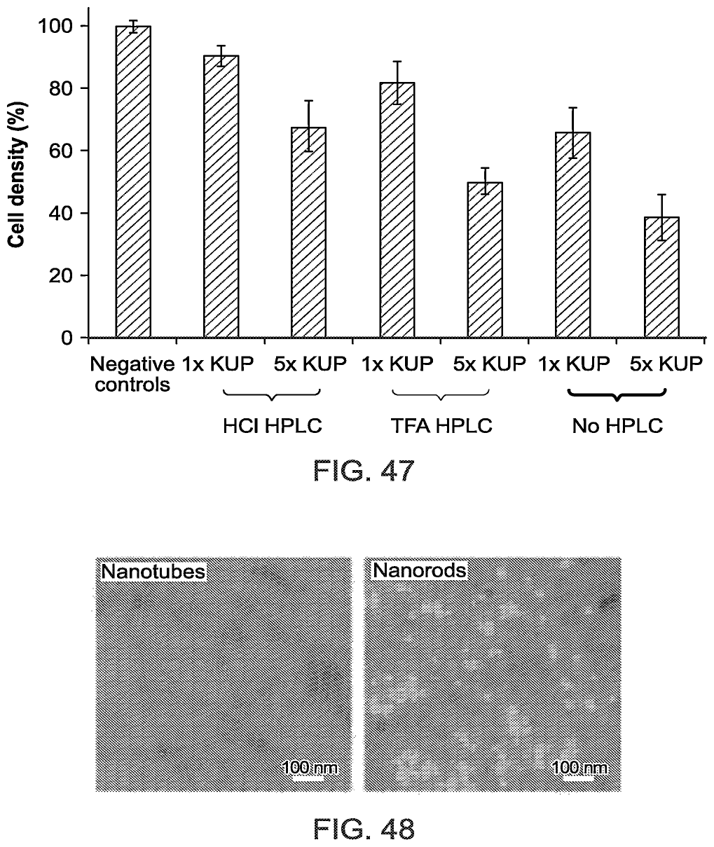

*Solvent A is H₂O, Solvent B is 100% acetonitrile, and Solvent C is 0.05N hydrochloric acid. The cell toxicity was evaluated using a standard cell viability test. ATDC5 cells were treated with RNTs, and after 48 hours cell viability normalized to negative controls (as 100). Results are showed in FIG. 47. These results demonstrate successful isolation of modules using a modified HPLC purification method to obtain RNTs. Using HCl instead of TFA in this purification process avoided the presence of fluorine containing contaminates within the module, which contributed to the toxicity of the resulting nanotube. Thus, use of HPLC decreased the toxicity of RNTs and use of HCl versus TFA further decreased the cytotoxicity. Molecular modules, e.g, TBLs were therefore isolated by applying HCl in liquid chromatography purification. This purification scheme is applicable for module I compounds (for RNT assembly and for module II compounds for TBL assembly) to yield functional Nanopieces with low toxicity.

### Example 11.2

Conversion of nanotubes (such as RNTs and TBLs) into nanorods was accomplished according to a process called “processing-1” (FIG. 53). In Processing-1, nanotubes are converted into short and homogeneous nanorods. This is very important to produce Nanopieces small enough to penetrate some types of tissue matrices for introduction of therapeutics into the tissue. Conversion of nanotubes to nanorods can be accomplished by altering pH, temperature, and/or using physical methods (such as sonicating, heating and blending (e.g. homogenizer)), and/or addition of aromatic chemicals. Different sizes of Nanopieces can be produced (FIGS. 5, 6 and 48). Based on the Nanopiece assembly mechanism, the processing approach may include at least one of the following: 1) before assembly, controlling the length and bundle of RNTs via changing physical and/or chemical conditions such as temperature, molecule motion and/or vibration (like sonication) and pH; 2) during assembly, adjusting assembly conditions via changing physical and/or chemical conditions including concentrations, pH and ionic strength to enhance and/or reduce the formation and stacking of Nanopieces; 3) after assembly, breaking long or stacked Nanopieces by via changing physical and/or chemical conditions including enhancing molecule motion/vibration (like sonication).

### Example 11.3

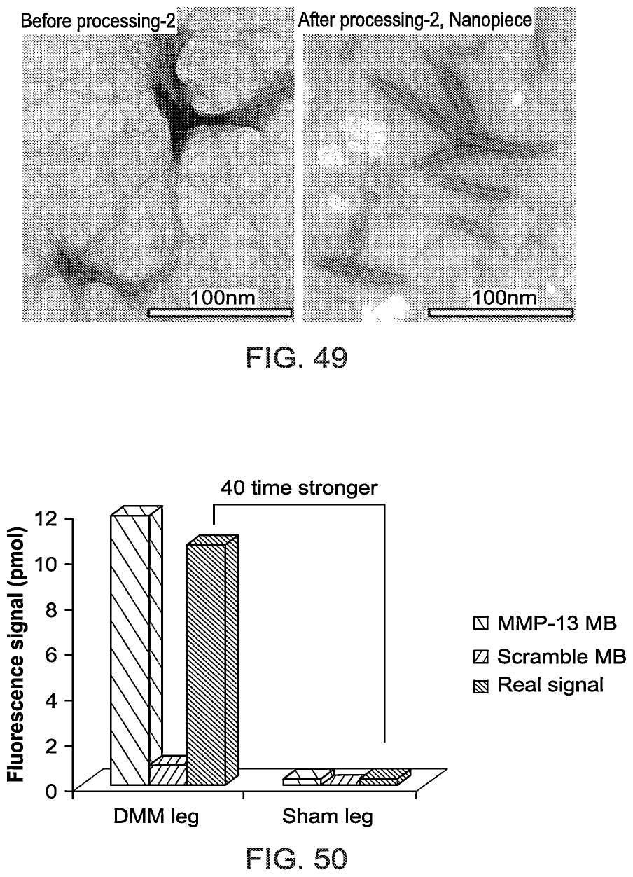

Preparation of Nanopieces was accomplished by a process called “processing-2” (FIG. 55). Processing-2 occurs after the incorporation between nanotubes or nanorods with delivery cargo and formation of bundles, ribbons or other agglomerates. These agglomerates can then be transformed to Nanopieces (FIG. 49). The size of the Nanopieces can be changed with changes in pH, ionic strength, temperature and concentration (FIGS. 4, 7-9).

FIGS. 15-23 and 26-32 demonstrated the successful tissue delivery after combining the above methods in Examples 11.1-11.3.

### Example 11.4

Preparation of small and large lipid Nanoparticles was accomplished using the procedures described below.

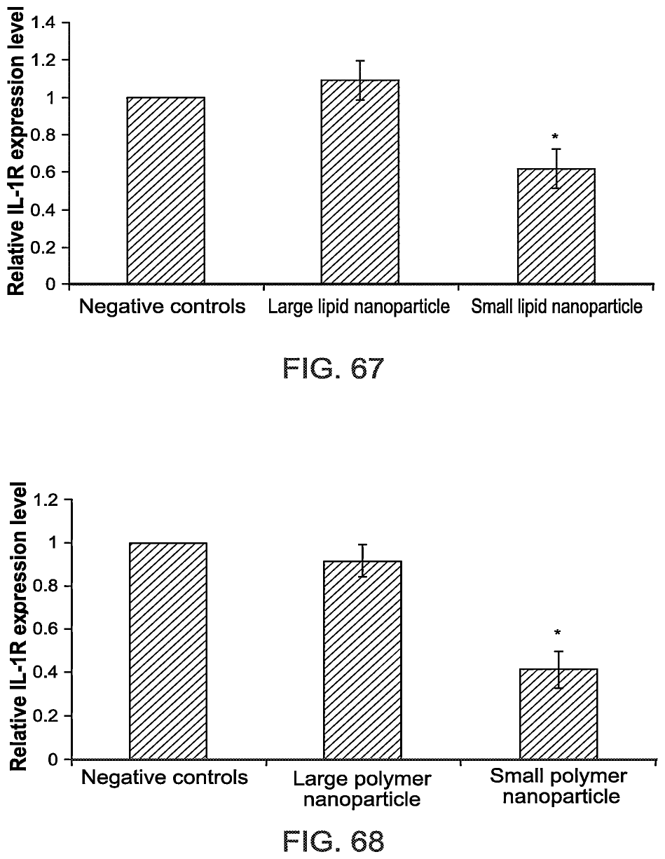

Preparation of large lipid nanoparticles with IL-1R siRNA (sphere shape 110 nm to 180 nm diameter):
1) Dissolve siRNA in 20 mM citrate buffer (pH 5.0, nuclease free) to achieve a concentration of 5004.
2) Dissolve DSPC, cholesterol, DODMA, and DSG-PEG (20:48:2:30 molar ratio) in absolute, anhydrous ethanol, and then add nuclease free water to achieve a concentration of 90% ethanol.
3) The total concentration of lipid in solution is then adjusted to 20 mM.
4) 1 μL of siRNA and 1 μL of lipid solutions are heated to 37° C., then mix at the same temperature and dilute with 8 uL nuclease free water. Sit at least 30 minutes before use.
   Preparation of small lipid Nanoparticles with IL-1R siRNA (sphere shape 70 nm to 120 nm diameter):
1) Dissolve siRNA in 10 mM citrate, 30 mM NaCl (pH 6.0, nuclease free) to achieve a concentration of 50 μM.
2) Dissolve DSPC, DSG-PEG, cholesterol, SPDiOC18, and DOTMA (10:10:39.8:0.2:40 molar ratio) in absolute, anhydrous ethanol, and then add an aqueous buffer (50 mM citrate, pH 4.0, nuclease free) to achieve a final concentration of 40% ethanol.
3) The total concentration of lipid in solution is then adjusted to 20 mM.
4) Extrude the lipid solution through two nuclepore polycarbonate filters (100 nm, 10 passes).
5) 1 μL extruded lipid solution and 1 μL siRNA are mixed under constant vortex, then dialyzed in PBS overnight to increase the pH to about 7.4.
   FIG. 67 shows successful localization/delivery of cargo to cartilage tissue using nucleic acid-loaded lipid nanoparticles. The small siRNA lipid nanoparticles localized to, penetrated cartilage tissue, and inhibited expression of the target gene.

### Example 11.5

Preparation of small and large polymer Nanoparticles was accomplished using the procedures described below.

Preparation of large and small polymer Nanoparticles with IL-1R siRNA:
1) Dissolve poly-lysine (PLL) (molecular weight, 15 kDa-30 kDa) in nuclease free water to 0.2 mg/mL.
2) Dialyze to remove salt (HBr).
3) Lyophilize.
   To prepare large PLL/siRNA nanoparticles (100-250 nm diameter):
1) Dissolve siRNA and PLL in 0.15M NaCl to concentrations of 10 μM and 2504, respectively.
2) Quickly add 1 uL 50 μM siRNA solution to 15 uL 100 μg/mL PLL and pipette well at room temperature.
3) Pipette and let sit for at least 30 minutes before use.
   To prepare small PLL/siRNA nanoparticles (50-75 nm diameter):
1) Dissolve siRNA and PLL in nuclease free water to concentrations of 50 μM and 100 μg/mL, respectively.
2) Quickly add 1 uL 50 μM siRNA solution to 15 uL 100 μg/mL PLL and pipette well at room temperature.
3) Use within 30 minutes of reaction.
   FIG. 68 shows successful localization/delivery of cargo to cartilage tissue using nucleic acid-loaded polymer nanoparticles. The small siRNA polymer nanoparticles localized to, penetrated cartilage tissue, and inhibited expression of the target gene.

FIGS. 67 and 68 demonstrated the successful tissue delivery of the above prepared lipid or polymer nanoparticles. Animals were injected with prepared large/small lipid or polymer nanoparticles delivered with IL-1R siRNA to right knees of mice. (Animal left knees were used as negative controls). After 24 hours, euthanize animals were euthanized and their knee cartilage was collected for real time RT-PCR. These data indicate that cargo-loaded nanostructures such as RNTs comprising compounds of Formula I, TBLs comprising compounds of Formula II, as well as lipid nanoparticles, and polymer nanoparticles successfully deliver cargo to target tissues.

### Example 12

A Non-Invasive, Early, and Sensitive Detection of Osteoarthritis Through In Vivo Imaging of MMP-13 mRNA Levels by Molecular Beacon (MB) and Nanopiece Delivery Technology

MBs were designed to target MMP-13 or GAPDH mRNA with a fluorophore/quench pair using a mouse model. Scramble sequence MB (Scramble) was verified to not bind with any mouse mRNA via BLAST. To demonstrate in vitro delivery and validation; MBs were delivered into chondrocytes by Nanopieces. After stimulation with IL-1β for 24 hours, chondrocytes were co-transfected GAPDH (red) and scramble (green) MBs or GAPDH (red) and MMP-13 (green) MBs via Nanopieces. Real time RT-PCR and fluorescence microscopy were used to verify the stimulation of MMP-13 expression, and a successful fluorescence signal resulted from using a MMP-13 MB.

Destabilization of the medial meniscus (DMM) surgery and in vivo delivery: DMM or sham surgeries were performed on 10-week-old 129SVE male mice to induce osteoarthritis. One week after surgery, MMP-13 and scramble MBs with different fluorophores delivered by Nanopieces were injected into knee joints of mice. Small animal fluorescence molecular tomography (FMT) was used to determine the fluorescence signal resulted from MMP-13 expression in the live animals for 3 weeks.

To test the in vitro efficacy of mRNA detection in chondrocytes using MBs delivered by Nanopieces, primary mouse chondrocytes were transfected with MBs either with or without IL-10 treatment. Before IL-10 treatment, the housekeeping GAPDH MB (red) was detected while the MMP-13 MB (green) was not. In contrast, after IL-10 treatment, both GAPDH MB (red) and MMP-13 MB (green) were detected, indicating the induction of MMP-13 mRNA levels by IL-10. Realtime rtPCR showed that MMP-13 mRNA level was up-regulated by about 10 times upon IL-10 stimulation. In contrast, Scramble MB transfection did not show any green fluorescence, suggesting that the fluorescence of MMP-13 MB was not due to non-specific degradation.

To evaluate in vivo efficacy, the following studies were carried out. After DMM surgery, MMP-13 MB was delivered intra-articularly to the knee joint of adult mice with Scramble MB that emits fluorescence at a different wave length than MMP-13 MB. Only a week after surgery, the DMM surgery leg displayed a strong MMP-13 signal than the contralateral Sham surgery leg (FIG. 2, left panel). In contrast, the Scramble MB showed very low fluorescence in both DMM and Sham surgery knee joints. After subtracting Scramble MB basal level signals, MMP-13 MB real signal was about 40 times stronger in the DMM leg than the sham leg. Such MMP-13 MB signals persist, even for 3 weeks after injection of MTh.

MMP-13 MB delivered by Nanopiece technology represents a sensitive tool to detect pro-inflammatory degenerative conditions as evidenced with chondrocytes in vitro and in OA animal models in vivo. This technology detects pathogenesis of OA at an early stage (within a week) in a mild OA model (DMM). A high sensitivity was achieved due to the detection at the mRNA level and the high efficiency of MB intracellular delivery by Nanopieces. The combination of molecular beacon and Nanopieces technology provided a powerful tool for early detection of OA in vivo in a specific and sensitive manner without harming any joint tissues.

Matrix metalloproteinases (MMP) are the major enzymes that degrade the components of the extracellular matrix during arthritis progression. MMP-13, which is usually produced by cartilage and bone, degrade interstitial collagens (types I, II and III) in both OA and RA. Expression of MMP-13 is low in normal cells, whereas in pathologic condition excess MMP-13 production is associated with inflammation. Thus, mRNA level of MMP-13 is useful as a diagnostic and prognostic tool for assessment of arthritis development. Therefore MMP-13 is recognized as a reliable target in early diagnosis of arthritis. These data indicate that intra-articular injection of Nanopieces+payload were successfully introduced into joint tissue and that the payload was functionally active after delivery.

The system and compositions described herein overcame the difficulty of accurately translating molecular beacon signal into MMP-13 mRNA expression level. MMP-13 upregulation pattern was demonstrated during OA progression using the Nanopiece—delivered beacons. Compared to earlier and current research and clinical methods, Nanopiece-Molecular Beacon technology achieved much earlier and more sensitive detection.

### Example 13

### Delivery of RNAi and Oligonucleotide Therapeutics and Diagnostics Via Self-Assembled Nanopieces™

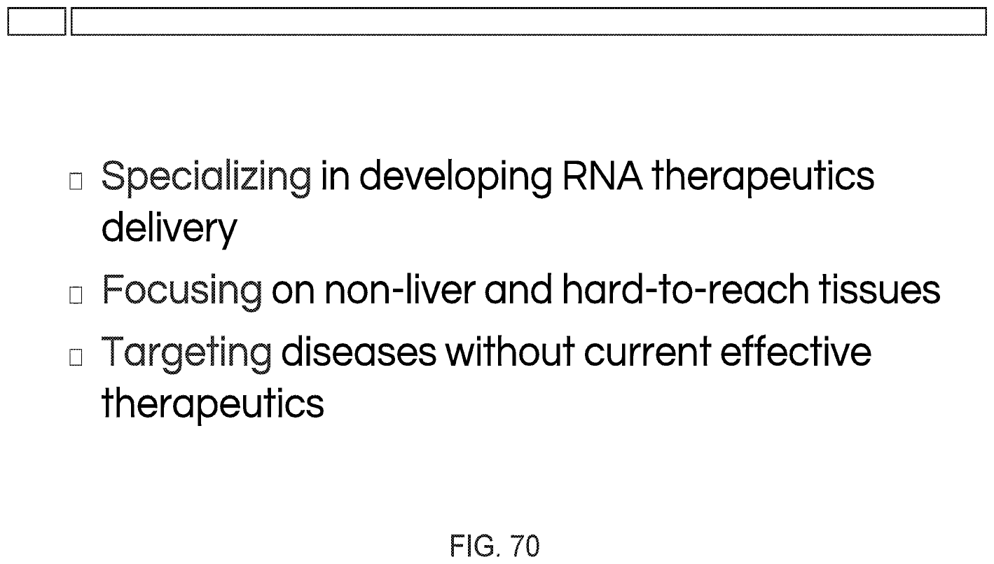

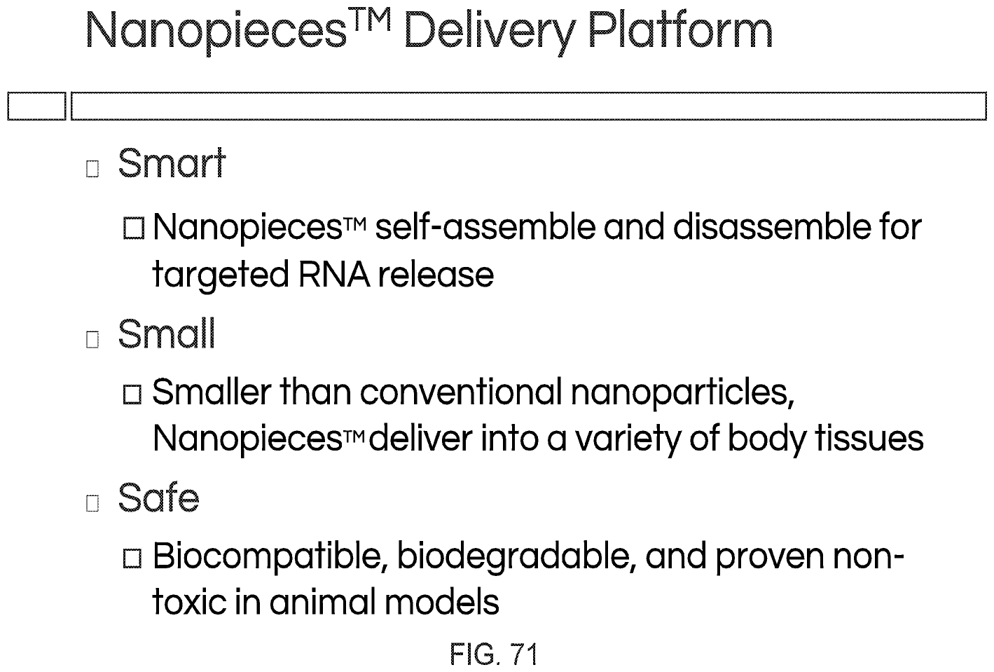

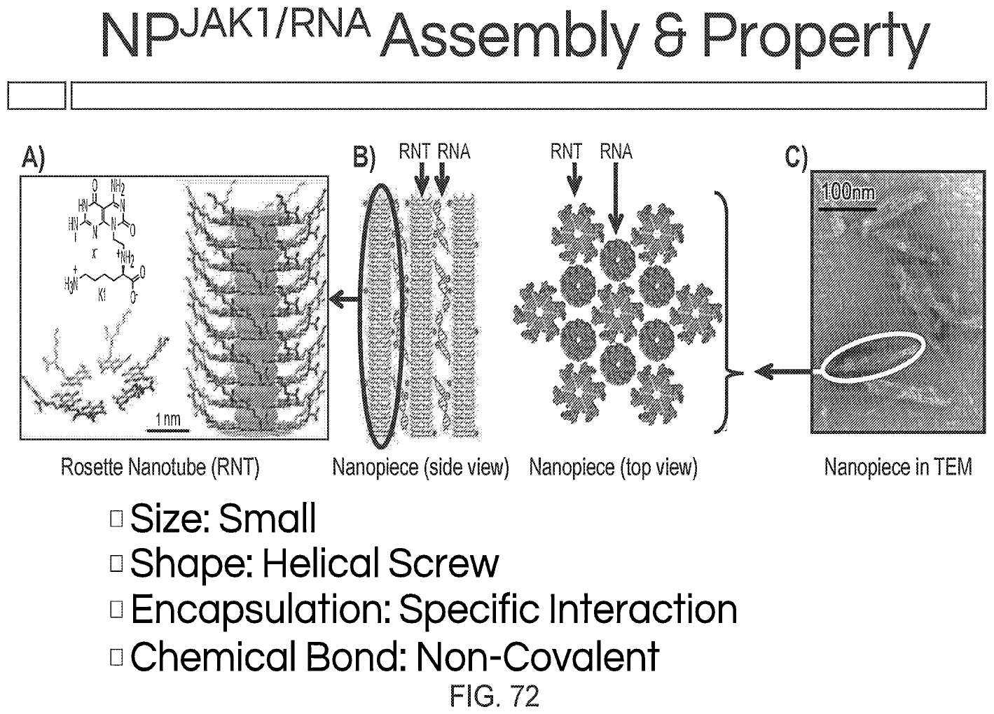

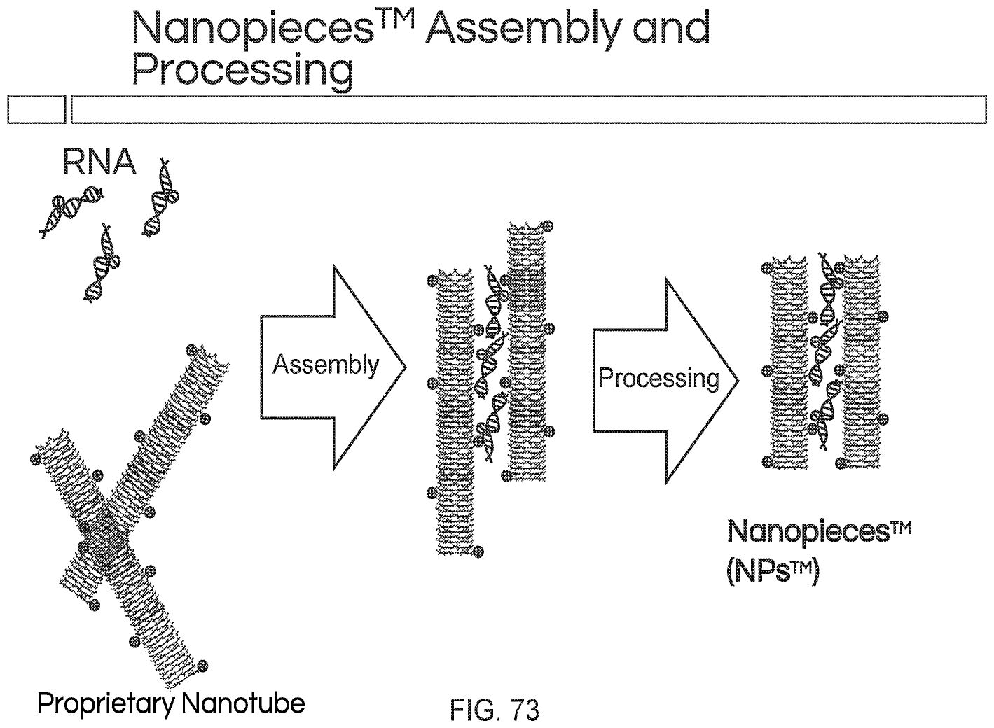

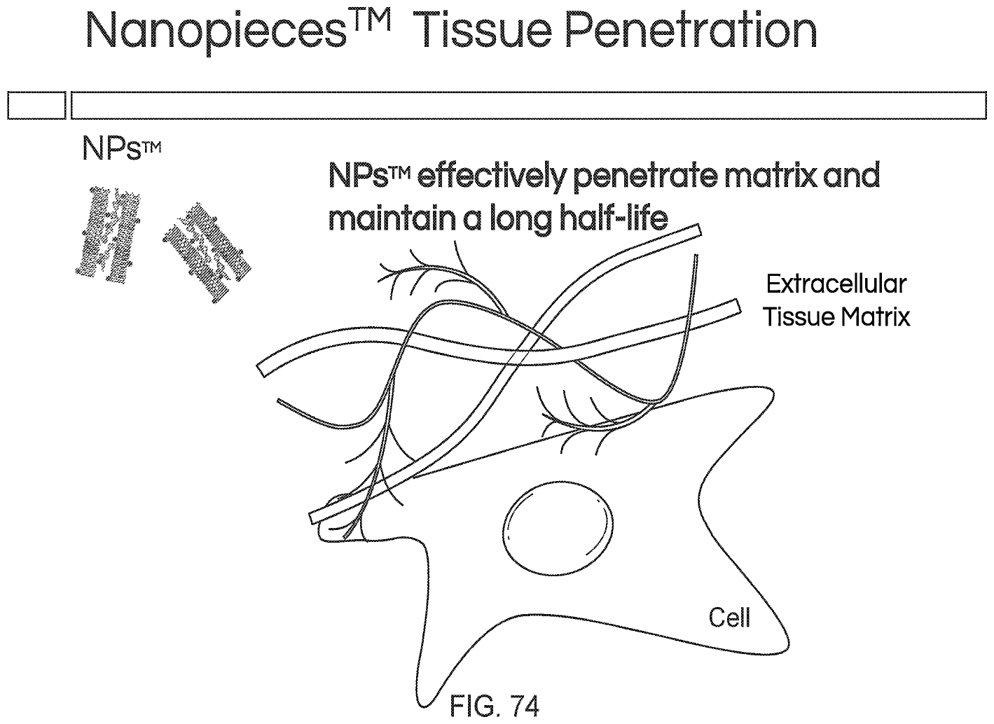

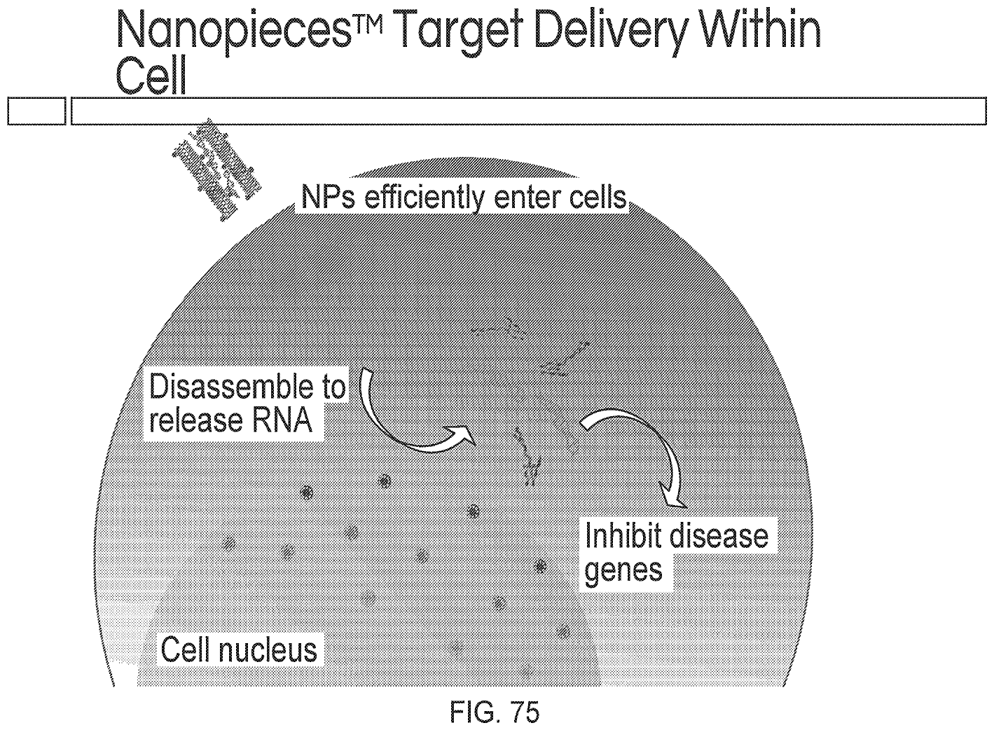

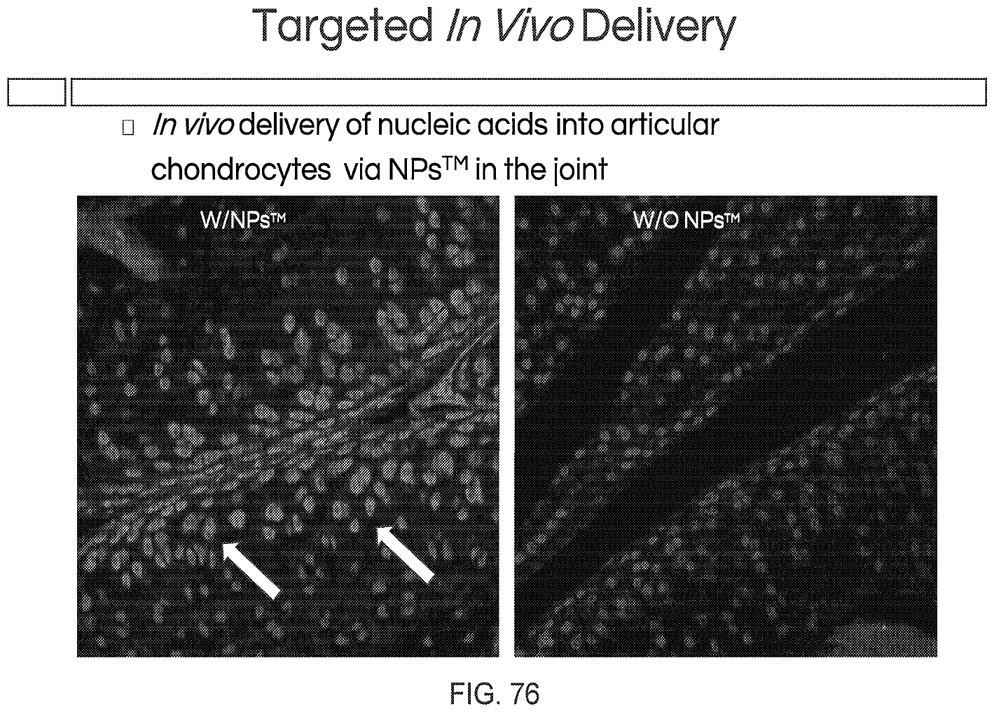

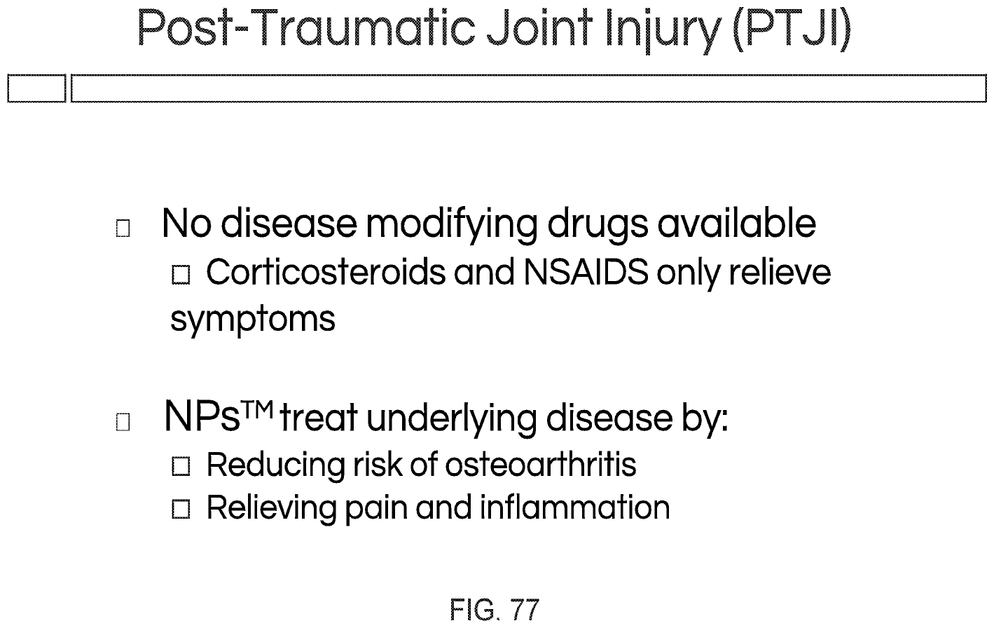

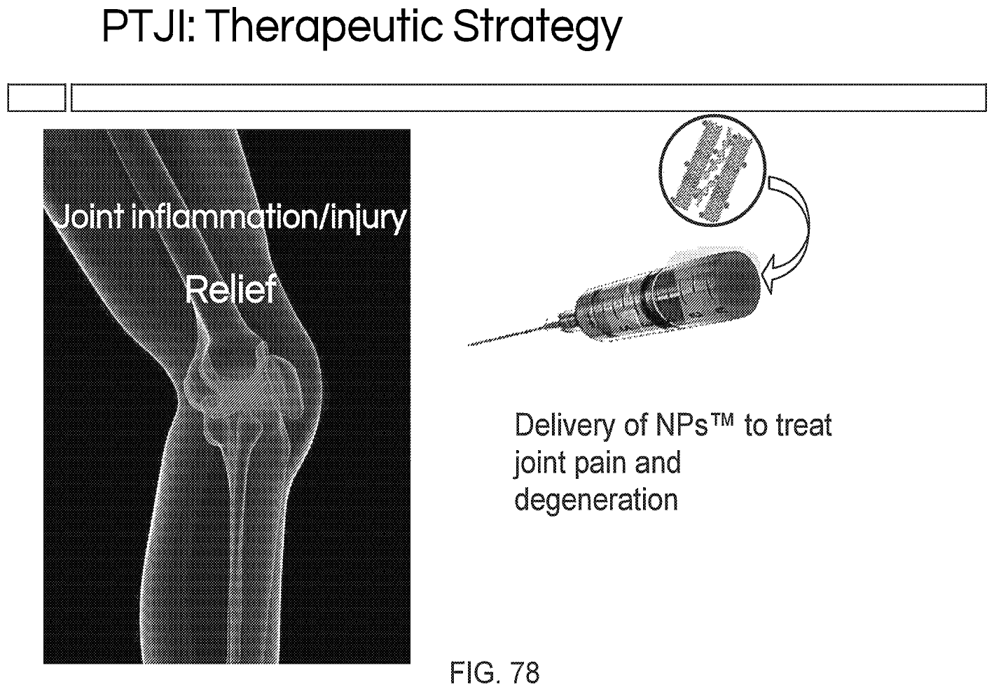

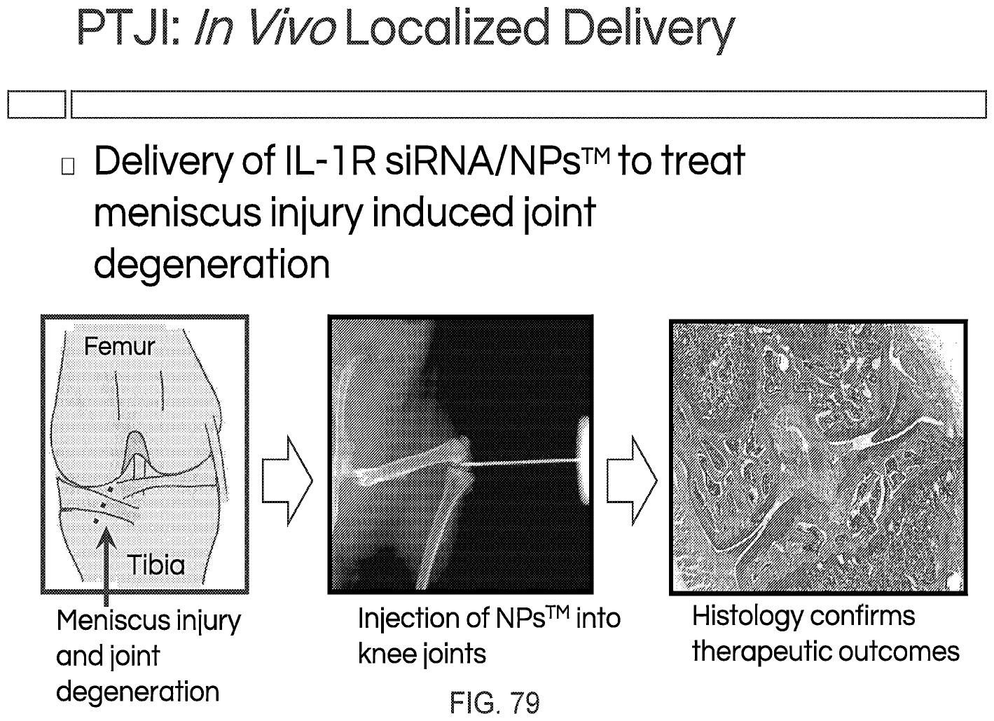

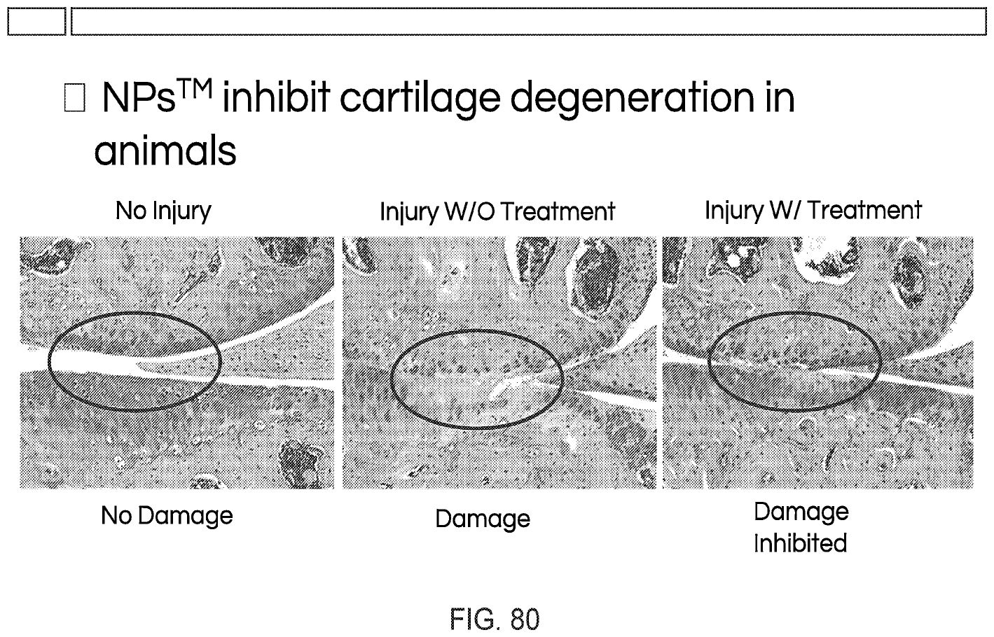

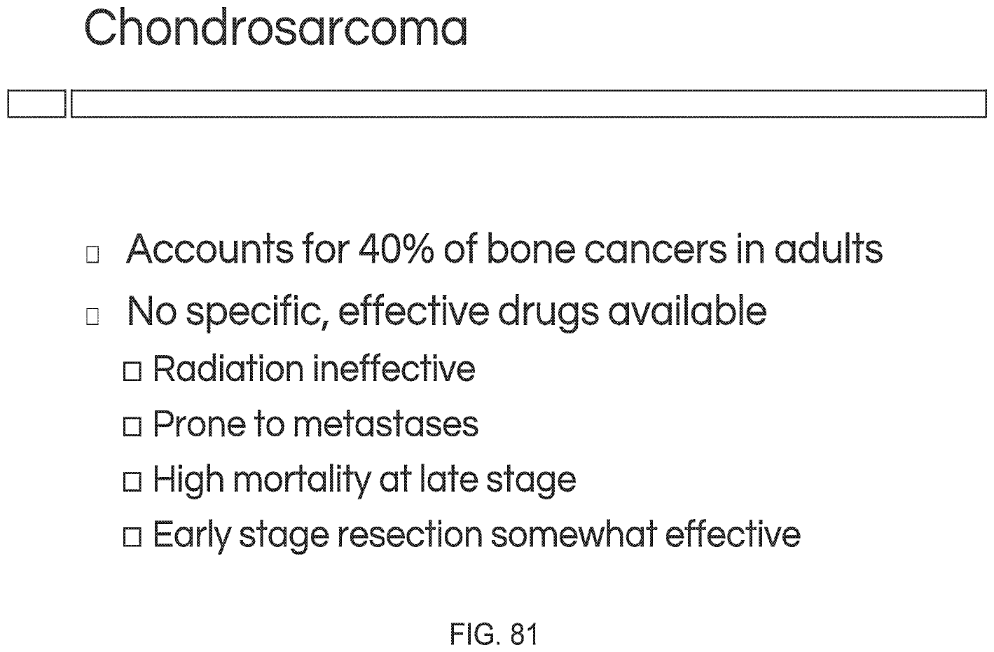

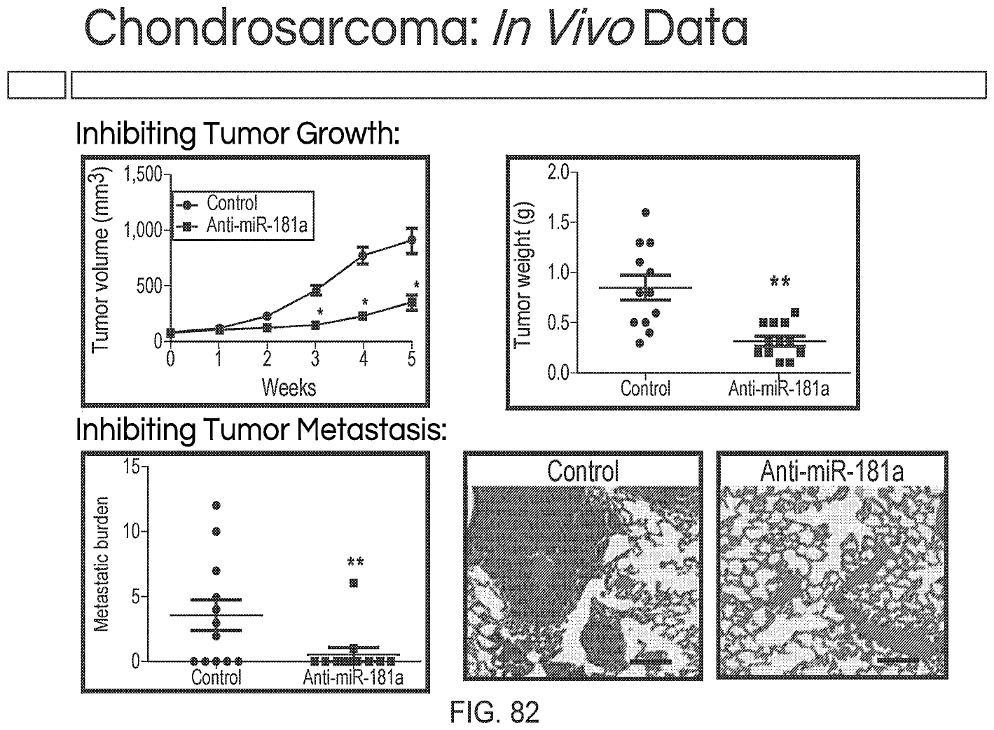

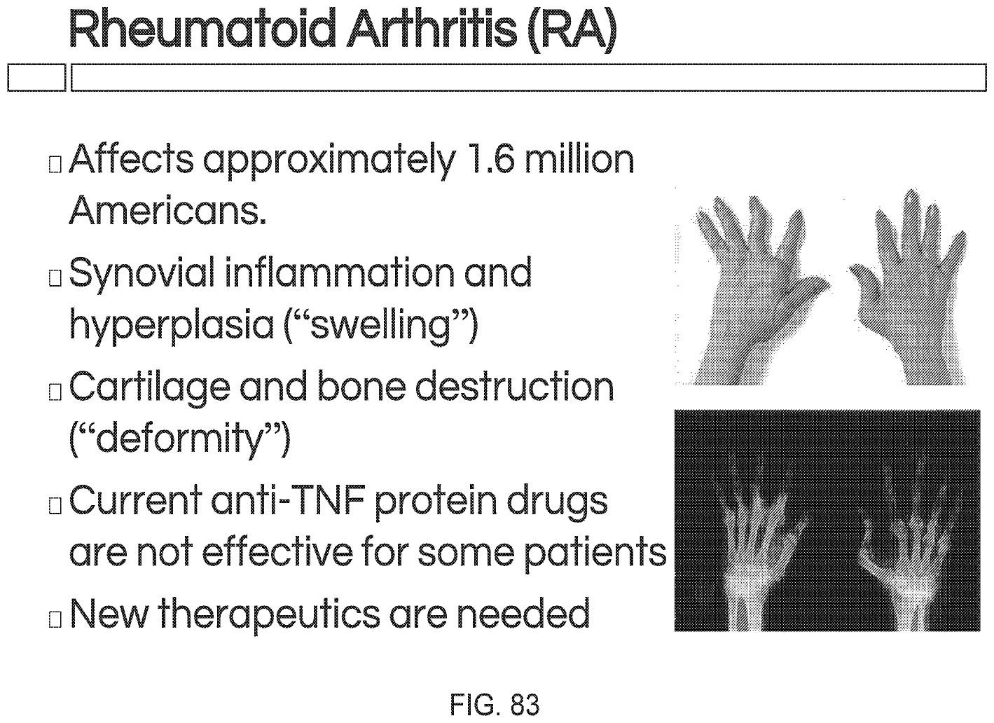

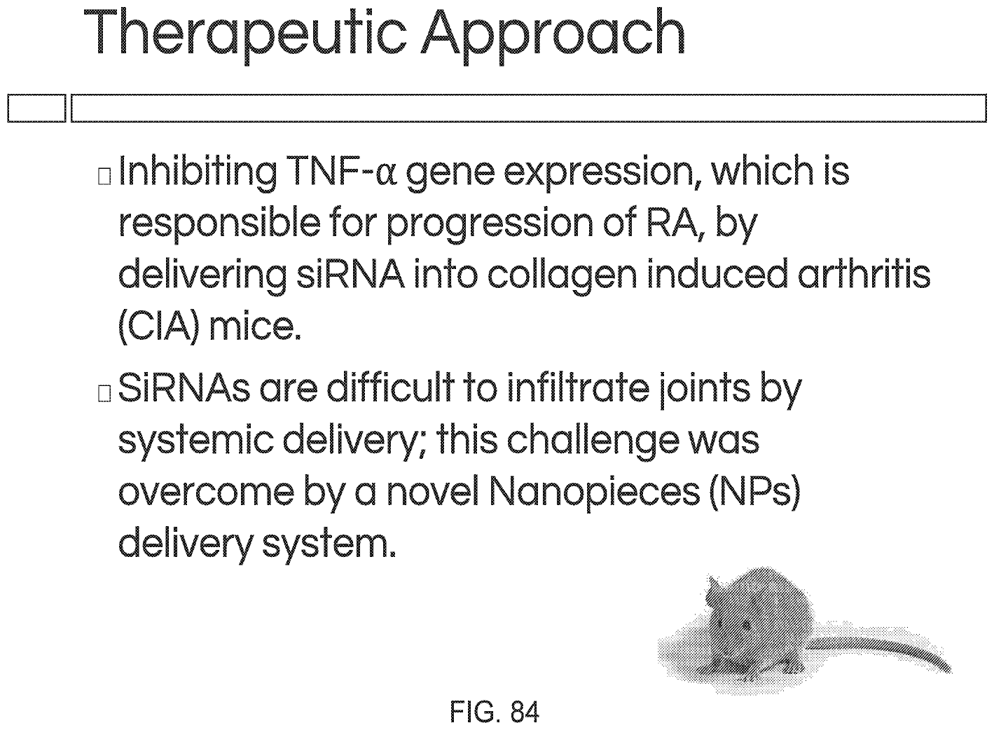

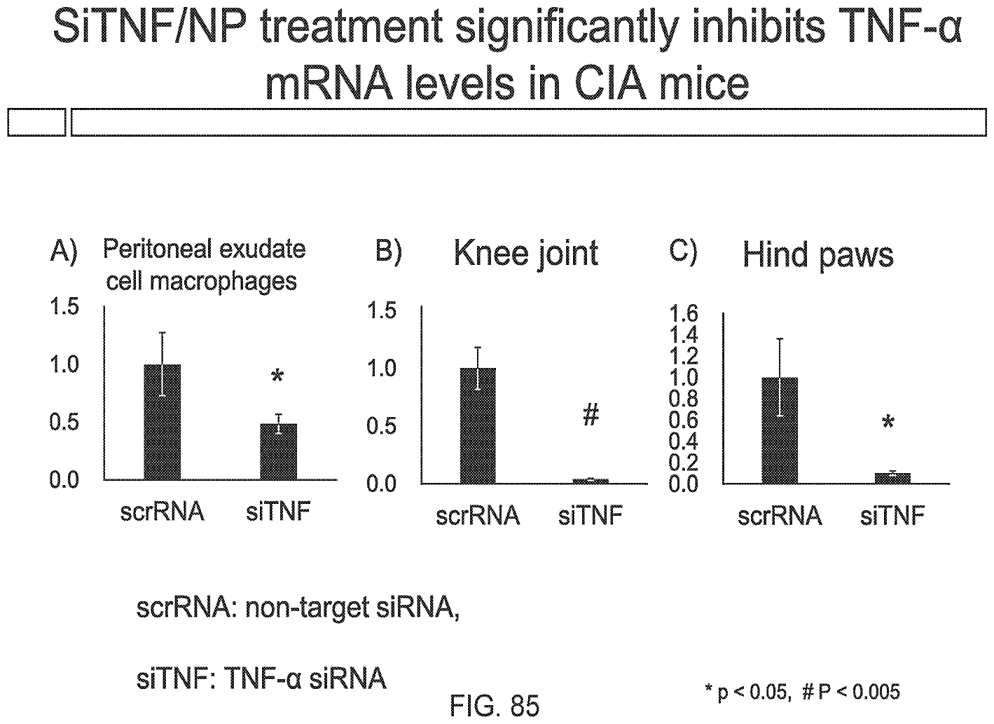

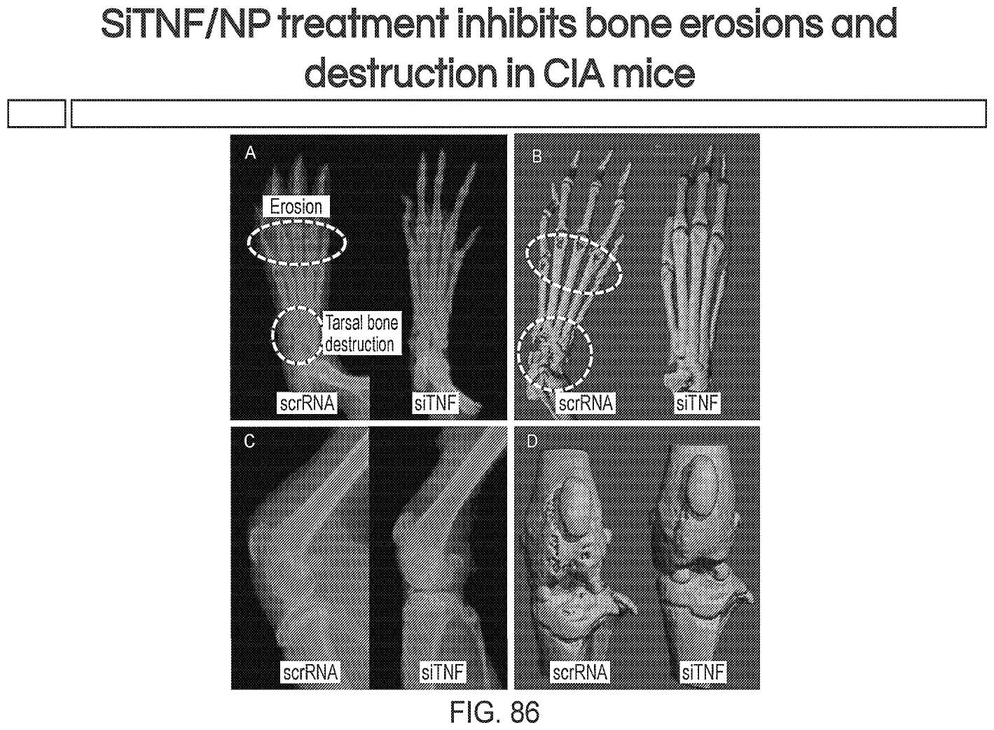

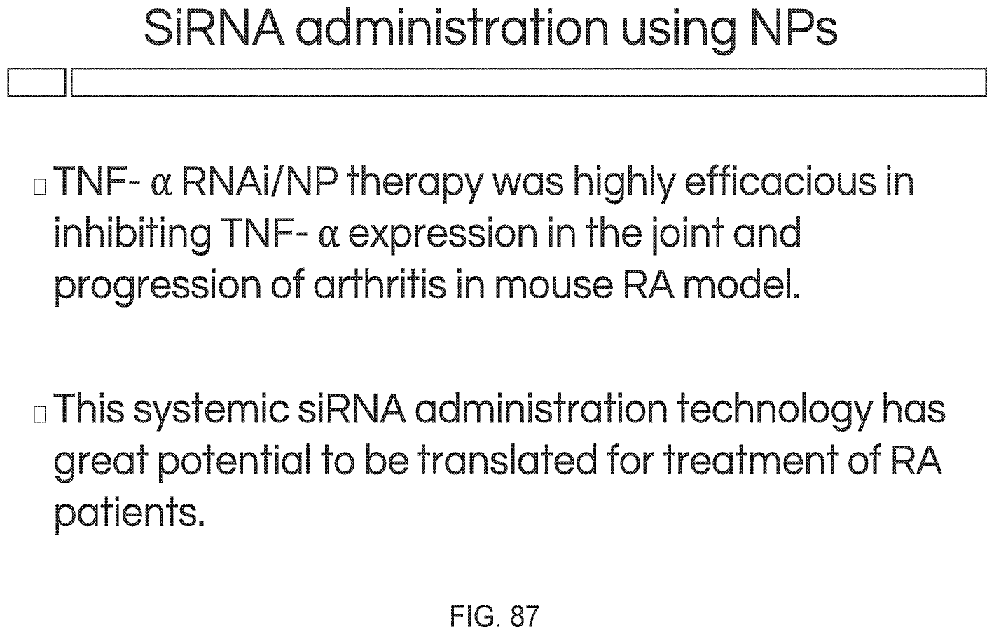

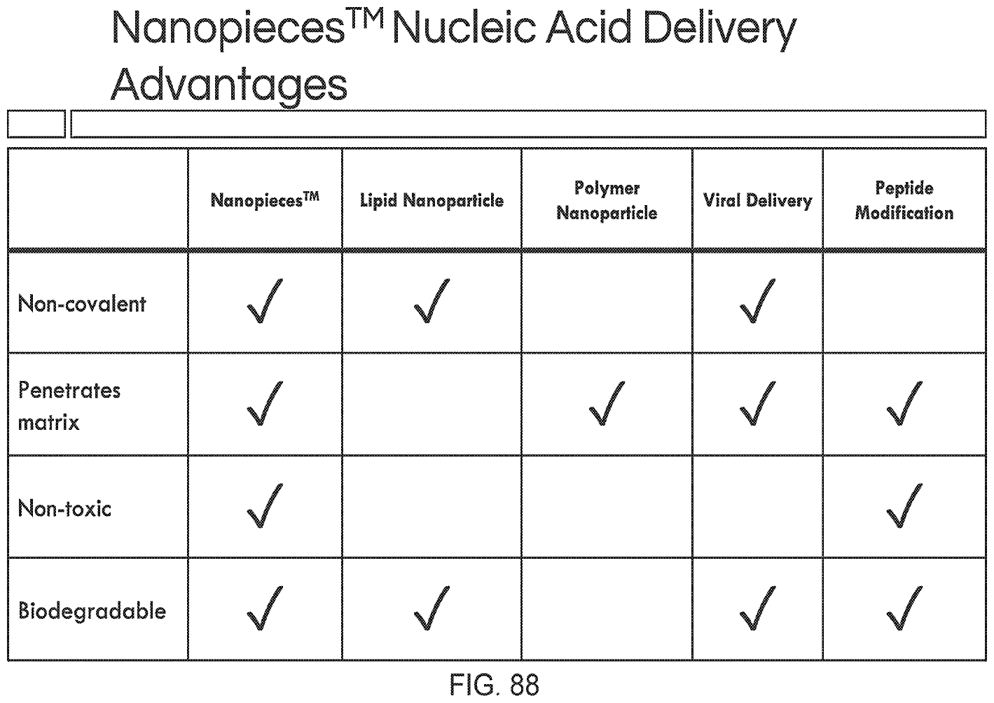

The compositions and methods described herein represent the next generation of therapeutics RNA delivery. Schematic self-assembled Nanopieces™ associated with RNAi (e.g., siRNA) and/or oligonucleotide for the use in therapeutics and diagnostics are shown in FIGS. 70-88.

Given the benefit of the above disclosure and description of exemplary embodiments, it will be apparent to those skilled in the art that numerous alternative and different embodiments are possible in keeping with the general principles of the invention disclosed here. Those skilled in this art will recognize that all such various modifications and alternative embodiments are within the true scope and spirit of the invention. While the invention has been illustrated and described in detail in the drawings and foregoing description, such illustration and description is to be considered as exemplary and not restrictive in character, it being understood that, only the preferred embodiments have been shown and described and that all changes and modifications that come within the spirit of the invention are desired to be protected. The appended claims are intended to cover all such modifications and alternative embodiments. It should be understood that the use of a singular indefinite or definite article (e.g., “a,” “an,” “the,” etc.) in this disclosure and in the following claims follows the traditional approach in patents of meaning “at least one” unless in a particular instance it is clear from context that the term is intended in that particular instance to mean specifically one and only one. Likewise, the term “comprising” is open ended, not excluding additional items, features, components, etc. References identified herein are expressly incorporated herein by reference in their entireties unless otherwise indicated.

### Example 14

### Systemic RNA Interference Therapy for Rheumatoid Arthritis Joints Through Delivery of Cargo-Loaded Nanopieces

The compositions and methods described herein can be used for systemic RNA interference (RNAi) therapy for arthritis (e.g., for rheumatoid arthritis-affected joints).

Rheumatoid arthritis (RA) is a disease which causes inflammatory synovitis and cartilage and bone destruction in the joints (McInnes I B, Schett G., The pathogenesis of rheumatoid arthritis. N Engl J Med. 2011 Dec. 8; 365(23):2205-19, and Choy E H, Panayi G S. Cytokine pathways and joint inflammation in rheumatoid arthritis. N Engl J Med. 2001 Mar. 22; 344(12):907-16. Review). In the past, Non-Steroidal Anti-Inflammatory Drugs (NSAIDs) and corticosteroid had been used to decrease the pain in patients with RA. Currently, disease-modifying anti-rheumatic drugs (DMARDs) (i.e. methotrexate) and anti-cytokine drugs are the most common therapeutics for RA (Okamura K, et al., Efficacy at 52 weeks of daily clinical use of iguratimod in patients with rheumatoid arthritis. Mod Rheumatol. 2015 July; 25(4):534-9, Okamura K, et al. Efficacy of the clinical use of iguratimod therapy in patients with rheumatoid arthritis. Mod Rheumatol. 2015 March; 25(2):235-40, Yonemoto Y, et al. Comparison of golimumab 100-mg monotherapy to golimumab 50 mg plus methotrexate in patients with rheumatoid arthritis: Results from a multicenter, cohort study. Mod Rheumatol. 2016; 26(1):24-8, Okamura K, et al., Evaluation of tocilizumab therapy in patients with rheumatoid arthritis based on FDG-PET/CT. BMC Musculoskelet Disord. 2014 Nov. 22; 15:393, and Okamura K, et al. The assessment of biologic treatment in patients with rheumatoid arthritis using FDG-PET/CT. Rheumatology (Oxford). 2012 August; 51(8):1484-91).

Anti-cytokine drugs such as tumor necrosis factor-α (TNF-α) antibody are targeting either the cytokine or the receptor. These play a regulatory role in the development and progression of RA and have been remarkable in improving the life quality of RA patients. Although most patients have lower disease activities using these drugs, there are still many patients who continue to suffer from joint destruction associated with RA because of the antibody against the protein drug or some adverse effects (van Schouwenburg P A, et al., Immunogenicity of anti-TNF biologic therapies for rheumatoid arthritis. Nat Rev Rheumatol. 2013 March; 9(3):164-72, and Yi H, et al. Induced production of anti-etanercept antibody in collagen-induced arthritis. Mol Med Rep. 2014 June; 9(6):2301-8). Thus, new therapeutic approaches other than protein drugs against RA are needed.

RNA interference (RNAi) therapy may be used for treatment (Kole R, et al., RNA therapeutics: beyond RNA interference and antisense oligonucleotides. Nat Rev Drug Discov. 2012 Jan. 20; 11(2):125-40, Inoue A, et al. Comparison of anti-rheumatic effects of local RNAi-based therapy in collagen induced arthritis rats using various cytokine genes as molecular targets. Mod Rheumatol. 2009; 19(2):125-33, Howard K A, et al., Chitosan/siRNA nanoparticle-mediated TNF-alpha knockdown in peritoneal macrophages for anti-inflammatory treatment in a murine arthritis model. Mol Ther. 2009 January; 17(1):162-8, Lee S J, et al. TNF-α gene silencing using polymerized siRNA/thiolated glycol chitosan nanoparticles for rheumatoid arthritis. Mol Ther. 2014 February; 22(2):397-408, Komano Y, et al., Arthritic joint-targeting small interfering RNA-encapsulated liposome: implication for treatment strategy for rheumatoid arthritis. J Pharmacol Exp Ther. 2012 January; 340(1):109-13, Khoury M, et al. Efficient new cationic liposome formulation for systemic delivery of small interfering RNA silencing tumor necrosis factor alpha in experimental arthritis. Arthritis Rheum. 2006 June; 54(6):1867-77, Khoury M, et al. Efficient suppression of murine arthritis by combined anticytokine small interfering RNA lipoplexes. Arthritis Rheum. 2008 August; 58(8):2356-67, Scheinman R I, et al., Functionalized STAT1 siRNA nanoparticles regress rheumatoid arthritis in a mouse model. Nanomedicine (Lond). 2011 December; 6(10):1669-82, Kanazawa T, et al., Systemic delivery of small interfering RNA targeting nuclear factor KB in mice with collagen-induced arthritis using arginine-histidine-cysteine based oligopeptide-modified polymer nanomicelles. Int J Pharm. 2016 Dec. 30; 515(1-2):315-323, and Luo X, et al. Adenovirus-Mediated Small Interfering RNA Targeting TAK1 Ameliorates Joint Inflammation with Collagen-Induced Arthritis in Mice. Inflammation. 2017 June; 40(3):894-903).

However, existing RNA interference-based therapies are limited in their use because of drawbacks and limitations such as the lack of an effective delivery vehicle. The compositions and methods of the invention overcome the drawbacks and limitations of earlier approached. To maximize the efficacy of RNAi therapy for RA, a systemic administration will have to be done without any adverse effects. The RNAi carrier must also have high delivery efficiency and specificity to appropriate targets of RNAi. Because of these requirements, it is necessary to explore new RNAi therapy for RA. The nanopiece-siRNA complexes described herein are characterized by such advantages.

The delivery vehicle and therapeutic described herein was a nucleobase derived nanotube complex that surrounds small interfering RNA (siRNA) named Janus Base with amine or lysine (K); JBaK nanopieces (NPs). These were consisting of siRNA and non-covalent nanotubes of a small biomimetic molecule.

Suppression of TNF-α through the RNAi method described herein regulated the synovial inflammation of the joints and decreased the joint destruction in a mouse RA model. siRNA was administered against TNF-α with JBaK NPs systemically in RA mouse model and evaluated the inflammation of the synovium and the destruction of joints.

### Example 14.1 Methods

Preparation of siRNA and JBaK NPs Complex

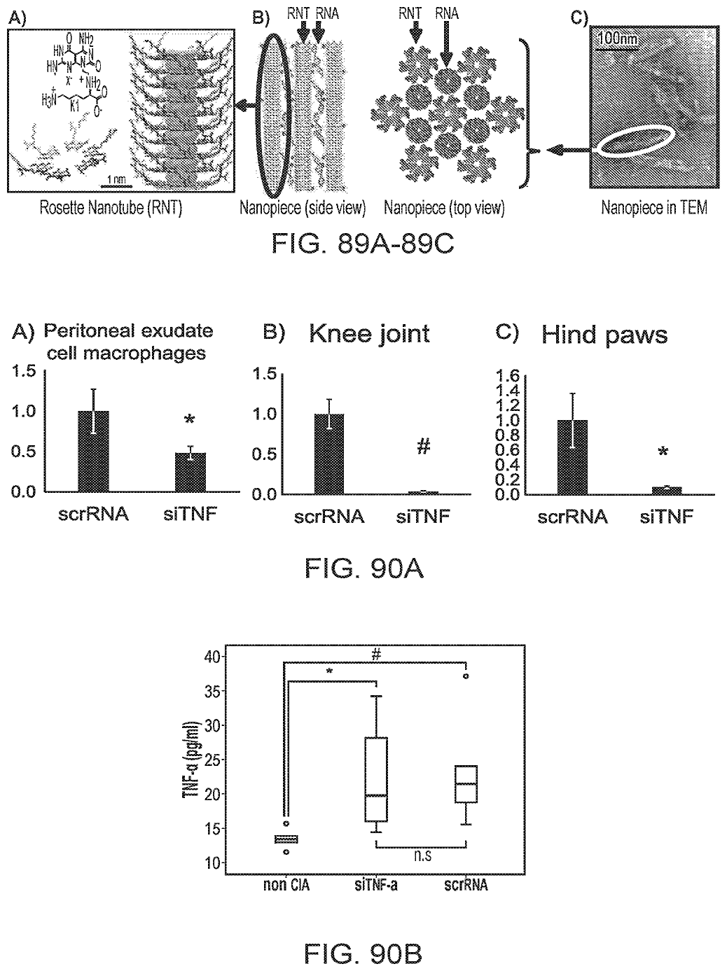

To deliver siRNA, a nucleobase derived nanotube complex named JBaK (Janus Base with amine or lysine (K)) was used. JBaK molecules was designed to combine two components: 1) nucleobase with hydrogen-bond donors and acceptors on two faces respectively, thus forming a Janus base, 2) a hydrophilic side chain containing amine or lysine. With this design, two faces of Janus base are complementary to each other to from a hydrophilic backbone, just like nucleobase; the amine or lysine containing side chains holding positive charge are stretching out to align the whole structure into a tubular shape (FIG. 89A-89C).

JBaK NPs were formed through a self-assembly process of JBaK nanotube and siRNA under specific conditions. JBaK nanotube was synthesized and verified through a 12-step total chemical synthesis procedure. JBaK nanotubes were formed using compounds of formula (I) or salts thereof. As shown in FIG. 89A, JBaK nanotubes were formed from a compound having a structure of

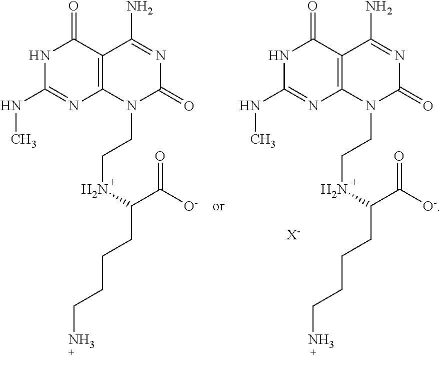

In Vitro Delivery of siRNA

siRNA was delivered to macrophages using the nanopiece-siRNA complex (siRNA cargo-loaded nanopieces). The macrophage cell line, RAW264.7, was purchased from ATCC and cultured with Dulbecco's Modified Eagle's Medium (DMEM) (#30-2002). To evaluate the in vitro TNF-α gene silencing efficacy in activated macrophages, the RAW264.7 cells were seeded in 12-well plate (100,000 cells/well) and cultured with Lipopolysaccharides (LPS)(100 ng) for 24 hours. Then, the activated RAW264.7 cells were transfected with siRNA for mouse TNF-α (siTNF) or mouse non-target siRNA (scrRNA) encapsulated within JBaK NPs. After 24 hours, total RNA was extracted from treated cells using an RNeasy RNA isolation kit (QIAGEN), then generated cDNA using iScript™ cDNA synthesis kit (Bio-Rad) according to the manufacturer's instructions. The cDNA of each sample was amplified by real-time quantitative PCR (RT-qPCR). The sequence of the primer used to detect mouse TNF-α were purchased from Integrated DNA technology (Iowa, USA) (forward 5′-AAG CCT GTA GCC CAC GTC GTA-3′ (SEQ ID NO: 229); reverse 5′-GGC ACC ACT AGT TGG TTG TCT TTG-3′ (SEQ ID NO: 230)). Relative transcript levels were calculated using the delta-delta Ct (ΔΔCt) method, normalized to rRNA 18S expression.

### Arthritis Induction for Collagen-Induced Arthritis

Collagen induced arthritis (CIA), a recognized model for rheumatoid arthritis, was generated in 8-week-old DBA/1J mice. 7 week-old mice were purchased from the Jackson Laboratory (JAX stock #000670) and housed under standard conditions at a temperature of 70° F., with 40-60% humidity and a 12 hour light/dark-cycle. CIA was achieved according to the manufacturer's instruction. Briefly, murine CIA was induced in 8 week-old male DBA/1J mice by single immunization with bovine type II collagen and complete Freund's adjuvant (Chondrex, USA) via subcutaneous injection. Arthritis develops 3.5-4 weeks after the first immunization.

### Arthritis Score and Paw Measurement

The mice were examined for signs of joint inflammation three times a week under anesthesia. The severity of arthritis was evaluated using the following clinical scoring method; 0, normal, 1, mild redness, slight swelling of ankle or wrist, 2, moderate swelling of ankle or wrist, 3, severe swelling including some digits, ankle, and foot, and 4, maximally inflamed. The clinical score was defined as the sum of the scores of all four paws of each mouse. In addition, the thickness of each paw was measured by digital caliper (Thermo Fisher Scientific, USA) at the same time as clinical assessment.

Systemic Delivery of siRNA

ON-TARGETplus siRNA (Dharmacon, USA) for mouse TNF-α (siTNF) or mouse non-target siRNA (scrRNA) were encapsulated within JBaK NPs and administered to CIA mice, via retro-orbital injections twice per week from 21 days to 49 days after the first induction of arthritis.

### Gene Expression Analysis of Mice Tissue, ELISA and Histology

At week 4, after twice injections of siRNA, the CIA mice were euthanized and paws were used to prepare the total RNA using an RNeasy RNA isolation kit (QIAGEN, USA). Peritoneal exudate cell macrophages (PECs) were also collected from each mice in the following method. 5 ml of ice-cold phosphate buffered saline was injected into abdominal space. After three minutes with mild massage, the injected fluid was aspirated with the syringe, then centrifuged at 4° C., 1,000 rpm for 5 min. Discard supernatant and re-suspend cell pellet in DMEM. The cells are allowed to adhere to the substrate by culturing them 2 hr at 37° C. Nonadherent cells are removed by gently washing three times with warm PBS.

At week 8, the serum TNF-α levels were measured by ELISA (Mouse TNF-alpha Quantikine ELISA Kit, R&D systems). The foot and knee were collected for the histological examination. TNF-α gene expression was quantified using real-time quantitative PCR using the QuantiTect SYBR Green PCR kit (Qiagen). The 18S and 36B4 ribosomal RNA were used for normalization. For the histological analyses, the decalcified knee joints were cut into thin thickness sections and H&E staining and Safranin-O and fast green staining were performed.

### Images and Bone Volume Analysis

X-ray images and high-resolution (10 μm isometric) 3D volume images using a desktop μCT scanner (MicroCT40, Scanco Medical. Tube Settings: 55 kVp and 145 μA. 300 ms integration time) were generated. Standard trabecular bone indices (e.g. bone volumetric density (Bone volume/Total volume; BV/TV), bone mineral density (BMD), trabecular number, trabecular thickness and trabecular separation of the subchondral bone) were calculated from manually-outlined volumes of interest in the distal femur, proximal tibia and calcaneus using the scanner's built-in analysis routines.

### Statistics

Statistical analysis was conducted using SPSS version 22 (IBM Inc., Chicago, IL, USA). Paired t-test, Mann-Whitney U test, One-way ANOVA and Turkey's post-hoc analyses were used for statistical analysis. Error bars represent one standard error (SE) of the mean.

### Example 14.2 Results

### In Vitro Delivery Optimization

The expression level of TNF-α mRNA were examined by RT-PCR and 50% suppression was observed in RAW264.7 cells with LPS stimulation.

### Knocking Down Efficacy for Mice TNF-α Gene Expression

After systemic delivery of siTNF in NPs via retro-orbital injections for two times, at the time of week 4, the TNF-α mRNA expression levels were significantly suppressed in peritoneal exudate cell macrophages (PECs) from abdominal cavity, knee joints, and hind paws in comparison to NP delivery of scrambled siRNA in CIA mice (FIG. 90A). NP systemic delivery achieved 96% (FIG. 90A, panel B (middle) and 90% (FIG. 90A panel c, right) knockdown of TNF-α mRNA levels in knee and hind paw joints respectively, indicating NP delivery can achieve highly efficient RNAi in joint tissues.

Serum TNF-α Levels in Each Treatment

At the end of systemic treatment with siRNA/NP, the serum TNF-α levels were measured with ELISA assay and there was no significant difference between siTNF treated mice and scrRNA treated mice (FIG. 90B).

### Clinical Evaluation of CIA Mice

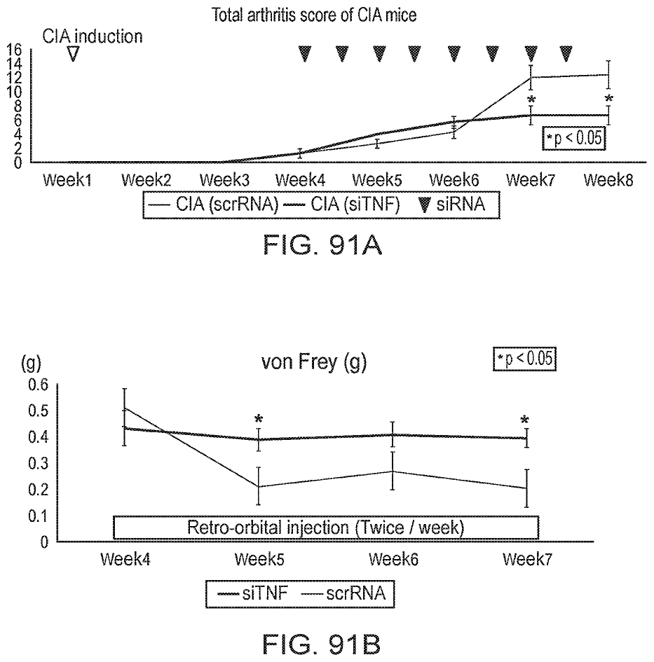

Total arthritis score was significantly reduced in siTNF treatment group in comparison with the sham treatment group (scrRNA) after CIA induction for 7 and 8 weeks (FIG. 91A). There were also decreased paw thickness in siTNF treated group in those weeks.

Evaluation of Nociception Under siRNA Delivery

To evaluate nociception under siRNA treatments, von Frey test were performed during the therapies. siTNF mice had higher mechanical nociception threshold than scrRNA mice (FIG. 91B).

### Evaluation of Joint Images and Bone Structure

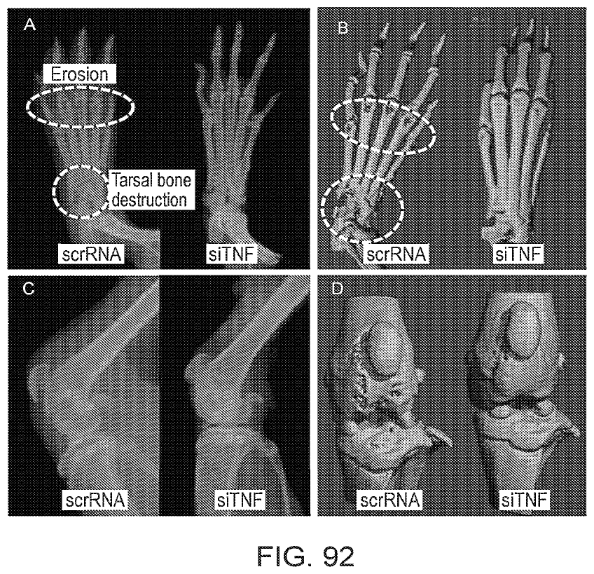

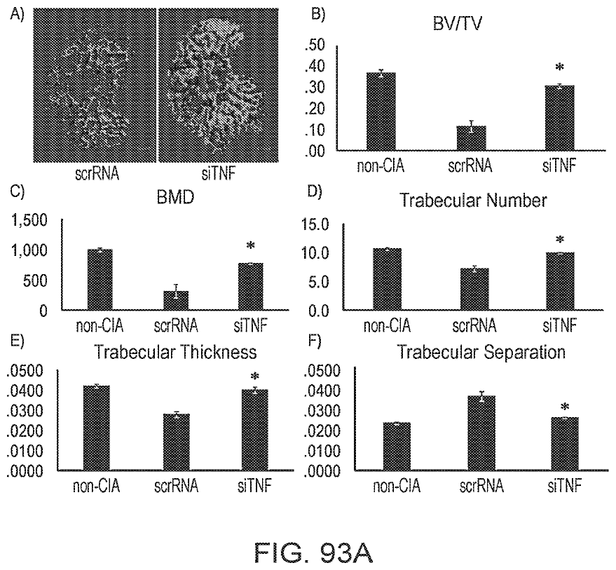

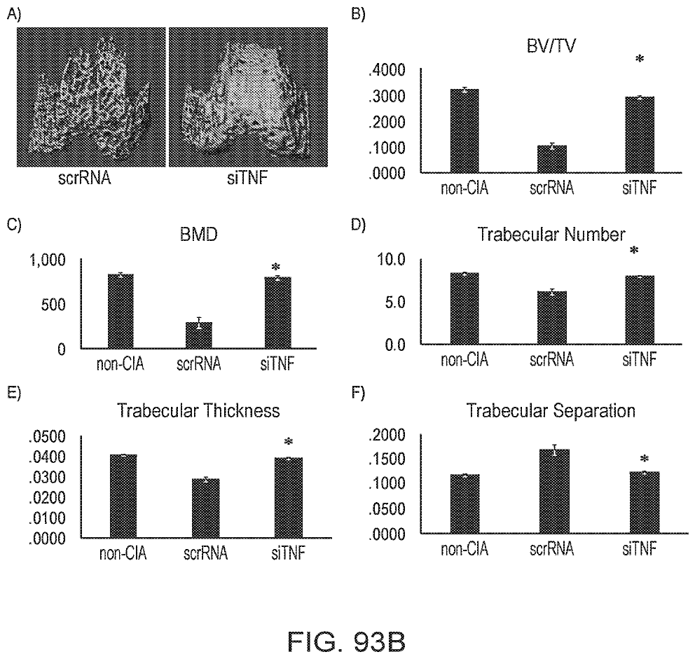

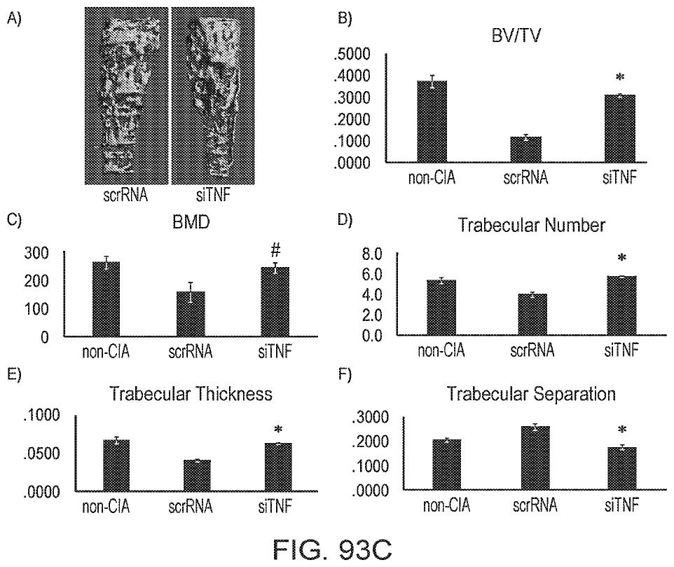

The siTNF treatment inhibited the bone erosions, and joint destructions (FIG. 92). From the quantitative analyses with μCT images of subchondral bone of tibia, the bone volume/total volume (BV/TV) with siTNF treatment was significantly higher than those with scrRNA (FIG. 93A, panel (b)). There were also significant improvement of bone mineral density (BMD) (FIG. 93A, panel (c)), trabecular number (FIG. 93A, panel (d)), trabecular thickness (FIG. 93A, panel (e)) and the trabecular separation (FIG. 93A, panel (f)) in siTNF mice compared to scrRNA mice. The same tendency was also observed with femur and calcaneus with each treatment mice (FIGS. 93B and 93C).

### Histological Analyses

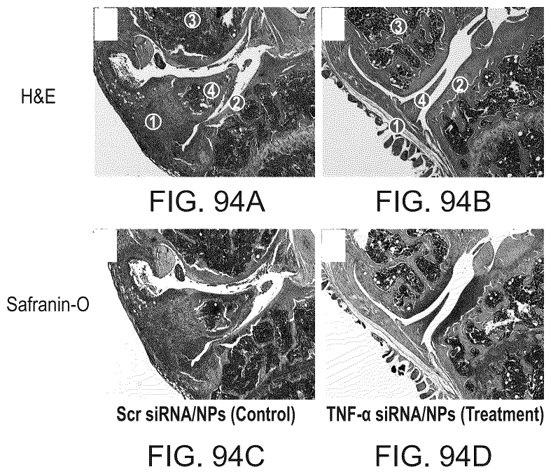

There were 1) synovial inflammation, 2) cartilage degradation, 3) bone loss, and 4) meniscus destruction in control CIA mice (FIG. 94A) and the siTNF treatment inhibited these changes (FIGS. 94B and 94D). Therefore, this treatment inhibited RA pathology in CIA mice.

### Example 14.3

The data demonstrated that 1) Systemic delivery of siRNA by NPs is achieved with high efficacy (>90%) in knocking down TNF-α gene expression in joint tissues, indicating its superiority in infiltrating peripheral joints for treatment of joint diseases such as the autoimmune disease, RA; and 2) Knocking down TNF-α mRNA in the joint tissues reduced the severity of inflammation and joint swelling, increased the threshold for the mechanical pain and inhibited bone erosion and reduction of joint destruction and BMD in arthritis mice.

CIA model mice (Takagishi K, et al., Effects of cyclosporin on collagen induced arthritis in mice. Ann Rheum Dis. 1986 April; 45(4):339-44, and Seeuws S, et al. A multiparameter approach to monitor disease activity in collagen-induced arthritis. Arthritis Res Ther. 2010; 12(4):R160 Epub 2010 Aug. 23) shares both immunological and pathological features with human RA, therefore it has been used extensively to study the pathogenesis of RA and for testing therapeutics. Previous reports have demonstrated siTNF therapy to CIA mice (Howard K A, et al., Chitosan/siRNA nanoparticle-mediated TNF-alpha knockdown in peritoneal macrophages for anti-inflammatory treatment in a murine arthritis model. Mol Ther. 2009 January; 17(1):162-8, Lee S J, et al. TNF-α gene silencing using polymerized siRNA/thiolated glycol chitosan nanoparticles for rheumatoid arthritis. Mol Ther. 2014 February; 22(2):397-408, Komano Y, et al., Arthritic joint-targeting small interfering RNA-encapsulated liposome: implication for treatment strategy for rheumatoid arthritis. J Pharmacol Exp Ther. 2012 January; 340(1):109-13, and Khoury M, et al. Efficient new cationic liposome formulation for systemic delivery of small interfering RNA silencing tumor necrosis factor alpha in experimental arthritis. Arthritis Rheum. 2006 June; 54(6):1867-77) Another report targeting interleukin (IL)-1, IL-6 and IL-18 simultaneously was also demonstrated (Khoury M, et al. Efficient suppression of murine arthritis by combined anticytokine small interfering RNA lipoplexes. Arthritis Rheum. 2008 August; 58(8):2356-67). The target of siRNA therapy was not only the cytokine but the other molecules involved in the inflammation such as Signal Transducers and Activator of Transcription (STAT)-1, nuclear factor-kappa B (NF-κB) and transforming growth factor beta-activated kinase 1 (TAK-1) (Scheinman R I, et al., Functionalized STAT1 siRNA nanoparticles regress rheumatoid arthritis in a mouse model. Nanomedicine (Lond). 2011 December; 6(10):1669-82, Kanazawa T, et al., Systemic delivery of small interfering RNA targeting nuclear factor KB in mice with collagen-induced arthritis using arginine-histidine-cysteine based oligopeptide-modified polymer nanomicelles. Int J Pharm. 2016 Dec. 30; 515(1-2):315-323, and Luo X, et al. Adenovirus-Mediated Small Interfering RNA Targeting TAK1 Ameliorates Joint Inflammation with Collagen-Induced Arthritis in Mice. Inflammation. 2017 June; 40(3):894-903). For the deliveries of these siRNAs, most used adenovirus, polymers, and lipid. From the point of the clinical application, these carriers were not enough to deliver siRNA safely to animal and/or human body. The compositions and methods described herein utilitz a nucleobase derived nanotube complex called JBaK Nanopieces (NPs) was used. This RNA carrier had high delivery efficiency and specificity to appropriate targets of RNAi in CIA mice. From the quantitative analyses for treated tissue with siTNF, there was the high efficiency of gene inhibition both in knee joints and hind paws, leading to the reduction of total arthritis score of CIA mice, validating the use of this NPs in the treatment of CIA mice.

Since these RNAi therapies utilizes encapsulated RNA rather than protein as a therapeutic, it circumvents the protein drug resistance problem (van Schouwenburg P A, et al., Immunogenicity of anti-TNF biologic therapies for rheumatoid arthritis. Nat Rev Rheumatol. 2013 March; 9(3):164-72) for some patients. Furthermore, RNAi therapy inhibits the synthesis of cytokines rather than neutralizing them after they are made, it may be used as an alternative approach for RA therapy in the cases protein drug is ineffective.

A large number of RA patients had suffered from fragile bone structures and related fractures during their entire stage of life. In order to overcome this situation, physicians need to treat the bone structure of patients with RA. In this study, CIA mice were used for the evaluation of bone microstructures. Seeuws S et al revealed that the cartilage and bone damages were observed using X-ray and μCT in CIA mice model (Seeuws S, et al. A multiparameter approach to monitor disease activity in collagen-induced arthritis. Arthritis Res Ther. 2010; 12(4):R160). They treated CIA mice with etanercept (ETN) or abatacept, both are biological DMARDs (bDMARDs), and these treatments reduced bone erosions in CIA mice.

Some previous reports demonstrated that the usage of bDMARDs had not increased, but prevented decreases in the bone mineral density (BMD) of the hip, spine and/or hand in human RA patients (Marotte H, et al. A 1-year case-control study in patients with rheumatoid arthritis indicates prevention of loss of bone mineral density in both responders and nonresponders to infliximab. Arthritis Res Ther. 2007; 9(3):R61, Wijbrandts C A, et al.,. Bone mineral density in rheumatoid arthritis patients 1 year after adalimumab therapy: arrest of bone loss. Ann Rheum Dis. 2009; 68(3):373-376, and Bertoldi 1, et al. Disease activity and bone density of mcp joints in patients with rheumatoid and psoriatic arthritis: is there a correlation?—a study in patients treated with methotrexate and an anti-TNF a agent). Regarding the mice RA model, Lee JH investigated the microstructure of tibial epiphysis (Lee J H, et al. Changes in microarchitectural characteristics at the tibial epiphysis induced by collagen-induced rheumatoid arthritis over time. Clin Intery Aging. 2012; 7:373-82) and Hyoju Y I et al evaluated the bone volume, BMD, and trabecular structure after the treatment with ETN for CIA mice and reported the improvement of bone loss in the knee joints of these mice (Yi H, et al. Induced production of anti-etanercept antibody in collagen-induced arthritis. Mol Med Rep. 2014 June; 9(6):2301-8). Lee S J et al also demonstrated that siTNF reduced the bone destruction and improved bone volume of hind paws and knee joints (Lee S J, et al. TNF-α gene silencing using polymerized siRNA/thiolated glycol chitosan nanoparticles for rheumatoid arthritis. Mol Ther. 2014 February; 22(2):397-408).

The data described herein showed that the siTNF therapy improved the bone volume of tibia, femur, and calcaneus in CIA mice and inhibited pathological changes in mice joints. Additionally, the results also revealed that the siRNA therapy improved the BMD and bone trabecular structures in those areas. These results indicated that systemic administration of siTNF with JBaK NPs not only decreased the arthritis score but improved the bone microstructure and joint component in RA murine model.

siTNF therapy increased the bone volume. The previous reports demonstrated that the increase of bone resorption in cancellous bone caused by upregulation of the expression of DKK-1 and regulation of the RANKL/RANK/osteoprotegerin (OPG) signaling pathway (Taketa T, et al. Selective cyclooxygenase-2 inhibitor prevents reduction of trabecular bone mass in collagen-induced arthritic mice in association with suppression of RANKL/OPG ratio and IL-6 mRNA expression in synovial tissues but not in bone marrow cells. J Bone Miner Metab. 2008; 26(2):143-51, Lubberts E, et al. Increase in expression of receptor activator of nuclear factor kappaB at sites of bone erosion correlates with progression of inflammation in evolving collagen-induced arthritis. Arthritis Rheum. 2002 November; 46(11):3055-64, Wu Q, et al. Secondary osteoporosis in collagen-induced arthritis rats. J Bone Miner Metab. 2016 September; 34(5):500-16, and Kato G, et al. The inhibitory effects of a RANKL-binding peptide on articular and periarticular bone loss in a murine model of collagen-induced arthritis: a bone histomorphometric study. Arthritis Res Ther. 2015 Sep. 12; 17:251), and the systemic OPG and anti-TNF-α antibody therapy prevented bone loss in CIA mice through distinct mechanisms involving decreased bone resorption and preserved bone formation (Saidenberg-Kermanac'h N, et al. TNF-alpha antibodies and osteoprotegerin decrease systemic bone loss associated with inflammation through distinct mechanisms in collagen-induced arthritis. Bone. 2004 November; 35(5):1200-7). The siTNF therapy can affect these signaling pathway(s).

Some reports demonstrated that treatments for RA rodent model improved the mechanical hypersensitivity in those animals (Gao X H, et al. A store-operated calcium channel inhibitor attenuates collagen-induced arthritis. Br J Pharmacol. 2015 June; 172(12):2991-3002 and Baddack-Werncke U, et al. Cytotoxic T cells modulate inflammation and endogenous opioid analgesia in chronic arthritis. J Neuroinflammation. 2017 Feb. 6; 14(1):30). Although there is no report about the change of nociception during siTNF treatment, the suppression of TNF-α expression has been reported to improve the neuropathic pain (Nakanishi M, et al. Go-sha-jinki-Gan (GJG) ameliorates allodynia in chronic constriction injury-model mice via suppression of TNF-α expression in the spinal cord. Mol Pain. 2016 Jun. 13; 12, Kim H K, et al., Pentoxifylline Ameliorates Mechanical Hyperalgesia in a Rat Model of Chemotherapy-Induced Neuropathic Pain. Pain Physician. 2016 May; 19(4):E589-600, and Li Y, et al., Curcumin attenuates diabetic neuropathic pain by downregulating TNF-α in a rat model. Int J Med Sci. 2013; 10(4):377-81). The results herein indicated that siTNF therapy with JBaK NPs pushed up the threshold of nociception and attenuated the mechanical hypersensitivity in CIA mice.

A difference in the knocking down rate of TNF-α between in vitro macrophage cell line and the tissue from the knee and hind paw of CIA mice was observed. The difference was that the cell line was the activated macrophages and the tissue contained other kinds of cells such as T cell and fibroblast. In the body of CIA model mice, those cells are positively and/or negatively stimulated with each other (Choy E H, Panayi G S. Cytokine pathways and joint inflammation in rheumatoid arthritis. N Engl J Med. 2001 Mar. 22; 344(12):907-16). Therefore, it might be possible the RNAi therapy herein with JBaK NPs acted to those cells at the peripheral joints, leading to the high-rate knocking down of TNF-α. Since the knocking down rate of TNF-α in the PECs from abdominal cavity was almost same to the results of macrophage cell line, the target of the current therapy might be not only macrophages but other immune-activated cells.

From the fact that the serum concentration of TNF-α was not changed at the end of 8 weeks while the RA progression were inhibited, the significance may be that a) this siRNA drug works differently than the protein drug which lowers the active TNF-α concentrations, b) it may have less side effects since TNF-α protein level was not overly suppressed, and c) it suggests that the potential mechanism of action may be early on, by inhibiting immune cells infiltration in the joint. Therefore, even if the TNF-α concentration recovers, the infiltration event has occurred earlier in the disease process and thus the disease process was inhibited even if TNF-α level was increased in the serum. Thus, knockdown TNF-α mRNA levels in the joint may be critical to inhibiting RA.

### Example 14.4

This data described herein demonstrate that RNAi/NP therapy is highly efficacious in inhibiting cytokine expression in the joint and progression of arthritis and bone destruction in mouse RA model. This systemic siRNA administration technology has great potential to treat RA patients if the results in humans match those in mice.

### Example 14.5

Relevant Abbreviations:

bDMARDs: biological DMARDs, BMD: bone mineral density, BV: bone volume, CIA: Collagen induced arthritis, DMARDs: disease-modifying anti-rheumatic drugs, DMEM: Dulbecco's Modified Eagle's Medium, ETN: etanercept, JBaK: Janus Base with amine or lysine (K), LPS: Lipopolysaccharides RA: rheumatoid arthritis, NF-κB: nuclear factor-kappa B, NPs: nanopieces, NSAIDs: Non-Steroidal Anti-Inflammatory Drugs, OPG: osteoprotegerin, RANK: Receptor activator of NF-κB, RANKL: Receptor activator of NF-κB ligand, PECs: Peritoneal exudate cell macrophages, RNAi: RNA interference, RT-qPCR: real-time quantitative PCR, scrRNA: non-target siRNA, siRNA: small interfering RNA, siTNF: siRNA for mouse TNF-α, STAT: Signal Transducers and Activator of Transcription, TAK: transforming growth factor beta-activated kinase, TNF-α: tumor necrosis factor-α, TV: total volume

1-25. (canceled)

26. A method of treating a joint disease comprising administration of an effective amount of a rosette nanopiece, wherein said nanopiece comprises i) a compound of Formula I or Formula II, 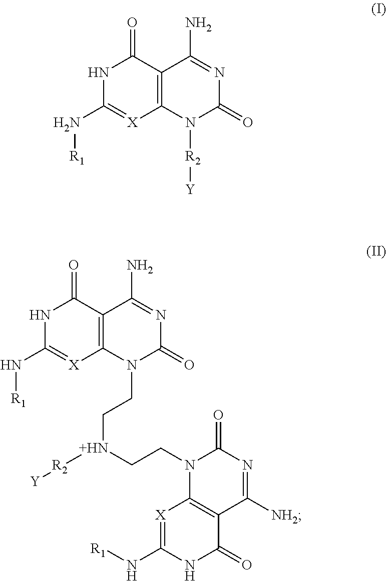
or a salt thereof, or a combination thereof, and
ii) a nucleic acid.

27. The method of claim 26, wherein said joint disease comprises a TNF-α-mediated autoimmune disease.

28. (canceled)

29. The method of claim 26, wherein said nanopiece comprises a TNF-α siRNA.

30. The method of claim 26, wherein said nanopiece enters a macrophage cell.

31. The method of claim 26, wherein said nanopiece is administered systemically.

32. (canceled)

33. The method of claim 26, wherein said joint disease comprises autoimmune, degenerative, inflammatory, infectious, cancerous, viral, fungal, injured, trauma, genetic, trauma, mechanical, nutritional or mal-alignment derived.

34. The method of claim 26, wherein said joint disease comprises osteoarthritis, juvenile onset of rheumatoid arthritis (JRA), reactive arthritis (RA), septic arthritis, tendinitis, or herniation.

35-45. (canceled)

46. The method of claim 26, 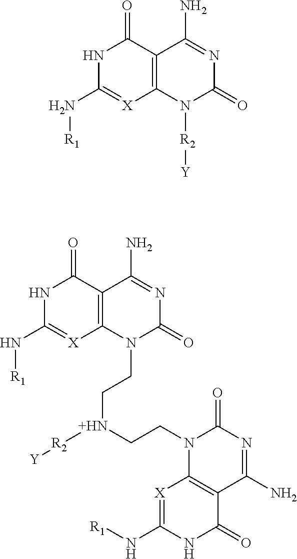
wherein, X is CH or N;
R₁ is hydrogen or aliphatic;
R₂ is hydrogen or a linker group; and
Y is absent when R₂ is hydrogen or is an amino acid side chain, amino acid or polypeptide.

47-49. (canceled)

50. The method of claim 26, wherein the nucleic acid comprises siRNA.

51. (canceled)

52. The method of claim 50, wherein the nucleic acid comprises the sequence: AAG CCT GTA GCC CAC GTC GTA (SEQ ID NO: 229) and GGC ACC ACT AGT TGG TTG TCT TTG (SEQ ID NO: 230).

53-54. (canceled)

55. The method of claim 26, wherein the nucleic acid is anti-miR-181a.

56. The method of claim 55, wherein the anti-miR-181a nucleic acid sequence comprises SEQ ID NO: 228 or SEQ ID NO: 229.

57. The method of claim 26, wherein the joint disease comprises a cancer in a joint.

58. The method of claim 57, wherein the cancer is sarcoma, hemangiopericytoma, connective tissue neoplasm, chondroma, or chondrosarcoma.
